(19) United States
(12) Patent Application Publication
(10) Pub. No.: US 2023/0257343 A1
(43) Pub. Date: Aug. 17, 2023

(54) NOVEL AMINO ACID DERIVATIVES

(71) Applicant: PeptiDream Inc., Kawasaki-shi (JP)

(72) Inventors: Naoki OKADA, Kawasaki-shi (JP); Kyosuke UEDA, Kawasaki-shi (JP); Masatoshi TAKUWA, Kawasaki-shi (JP); Shunichi NAKANO, Kawasaki-shi (JP); Kotaro TOKUMOTO, Kawasaki-shi (JP); Tomoko ASHIZAWA, Kawasaki-shi (JP); Shuhei YAMAKOSHI, Kawasaki-shi (JP); Yutaka KOBAYASHI, Kawasaki-shi (JP); Katsuma MATSUI, Kawasaki-shi (JP); Ayumu MATSUDA, Kawasaki-shi (JP); Masatoshi MATSUMOTO, Kawasaki-shi (JP); Keiichi MASUYA, Kawasaki-shi (JP); Atsushi YOSHIZAWA, Kawasaki-shi (JP); Masahiko KINEBUCHI, Kawasaki-shi (JP); Takeru EHARA, Kawasaki-shi (JP); Masami YAMADA, Kawasaki-shi (JP); Kouki MORIMOTO, Kawasaki-shi (JP); Yoshihide MIZUKOSHI, Kawasaki-shi (JP); Haruaki KURASAKI, Kawasaki-shi (JP); Motoki MURAI, Kawasaki-shi (JP); Kentarou FUKUMOTO, Kawasaki-shi (JP); Douglas Robert CARY, Kawasaki-shi (JP)

(21) Appl. No.: 18/109,702

(22) Filed: Feb. 14, 2023

Related U.S. Application Data
(60) Provisional application No. 63/435,552, filed on Dec. 28, 2022.
(60) Provisional application No. 63/396,385, filed on Aug. 9, 2022.
(60) Provisional application No. 63/310,905, filed on Feb. 16, 2022.

(51) Int. Cl. C07C 275/42; C07C 275/16; C07C 233/08; C07D 207/14; C07D 211/32; C07D 233/61; C07D 231/12; C07D 249/06; C07D 471/04; C07D 403/04

(52) U.S. Cl. CPC C07C 275/42; C07C 275/16; C07C 233/08; C07D 207/14; C07D 211/32; C07D 233/61; C07D 231/12; C07D 249/06; C07D 471/04; C07D 403/04

(57) ABSTRACT

A novel amino acid derivative is provided, wherein the amino acid derivative is expected to improve solubility of polypeptides comprising the derivative therein.

### FIELD OF THE DISCLOSURE

This disclosure relates to novel amino acid derivatives.

### BACKGROUND OF THE DISCLOSURE

The solubility of a polypeptide is primarily dependent on the physical properties of its amino acids. Amino acids can be classified as acidic, basic, polar uncharged, or non-polar (hydrophobic). It is known that polypeptides with a high content of non-polar amino acids or polar uncharged amino acids are preferentially solubilized by organic solvents such as DMSO, methanol, propanol, isopropanol, or DMF whereas overall acidic peptides can normally be reconstituted in basic solvents and overall basic peptides in acidic solvents.

### SUMMARY OF THE DISCLOSURE

Generally, however, demand for polypeptides with improved solubility remains high.

In one aspect, a novel amino acid derivative which is expected to improve the solubility of polypeptides is provided.

In another aspect, a novel amino acid derivative wherein one or more functional groups within the amino acid are protected by protecting groups is provided.

In a further aspect, an enantiomer of the novel amino acid derivative is provided.

### DETAILED DESCRIPTION OF THE DISCLOSURE

### Abbreviation

AcOH acetic acid (CAS: 64-19-7)
aq. aqueous
Boc tert-butyloxycarbonyl
Boc₂O di-tert-butyl decarbonate (CAS: 24424-99-5)
br broad
conc. concentrated
CPME cyclopentyl methyl ether (CAS: 5614-37-9)
CSA 10-Camphorsulfonic acid (CAS: 5872-08-2)
d doublet
DCM dichloromethane (CAS: 75-09-2)
dd double doublet
DIPEA, DIEA N,N-diisopropylethylamine (CAS: 7087-68-5)
DMAP 4-dimethylaminopyridine
DMF N,N-dimethylformamide (CAS: 68-12-2)
EtOAc ethyl acetate (CAS: 141-78-6)
Et ethyl
Et₃N triethylamine (CAS: 121-44-8)
Fmoc 9-fluorenylmethoxycarbonyl
Fmoc-OSu 2,5-dioxopyrrolidin-1-yl 9H-fluoren-9-ylmethyl carbonate (CAS: 82911-69-1)
h hour
HATU 1-[bis(dimethylamino)methylene]-1H-1,2,3-triazolo[4,5-b]pyridinium 3-oxide hexafluorophosphate (CAS: 148893-10-1)
IPE, iPr₂O diisopropyl ether (CAS: 108-20-3)
iPrOAc isopropyl acetate (CAS: 108-21-4)
iPrOH 2-propanol, isopropanol
iPr isopropyl
M molar
m multiplet
MeOH methanol (CAS: 67-56-1)
min minute
MOM methoxymethyl
MSA N-trimethylsilyl-N-methylacetamide (CAS: 7449-74-3)
MTBE methyl t-butyl ether (CAS: 1634-04-4)
MTHP 2-Methyltetrahydrofuran (CAS: 96-47-9)
N Normal
n-BuLi normal-butyllithium (CAS: 109-72-8)
Pd₂(dba)₃ Tris(dibenzylideneacetone)dipalladium(0) (CAS: 51364-51-3)
Pd(PPh₃)₄ Tetrakis(triphenylphosphine)palladium(0) (CAS: 14221-01-3)
Ph phenyl
quant quantitative
RP-HPLC reverse-phase HPLC
rt room temperature
s singlet
satd. saturated
SPhos dicyclohexyl(2′,6′-dimethoxy[1,1′-biphenyl]-2-yl)phosphane (CAS: 657408-07-6)
t triplet
tert tertiary
TFA trifluoroacetic acid (CAS: 76-05-1)
THF tetrahydrofuran (CAS: 109-99-9)
BSA N,O-Bis(trimethylsilyl)acetamide (CAS: 10416-59-8)
DMAP 4-dimethylaminopyridine (CAS: 1122-58-3)
DME 1,2-dimethoxyethane (CAS: 110-71-4)
EDCI·HCl ({[3-(dimethylamino)propyl]imino}methylidene)(ethyl)amine hydrochloride (CAS: 25952-53-8)
iPrOH 2-propanol, isopropanol (CAS: 67-63-0)
MeCN acetonitrile (CAS: 75-05-8)
MTBE methyl tert-butyl ether (CAS: 1634-04-4)
Pd(dppf)Cl₂ Bis(diphenylphosphino)ferrocene]dichloropalladium(II) (CAS: 72287-26-4)
t-BuOH tert-Butyl Alcohol, 2-Methyl-2-propanol (CAS: 75-65-0)
THF tetrahydrofuran (CAS: 109-99-9)
T3P 1,3,5-tripropyl-1lambda5,3lambda5,5lambda5-triphosphinane-1,3,5-trione (CAS: 68957-94-8 as 50% solution in EtOAc)

### [A Novel Amino Acid Derivative]

The novel amino acid derivative of this disclosure is at least one selected from the group consisting of:

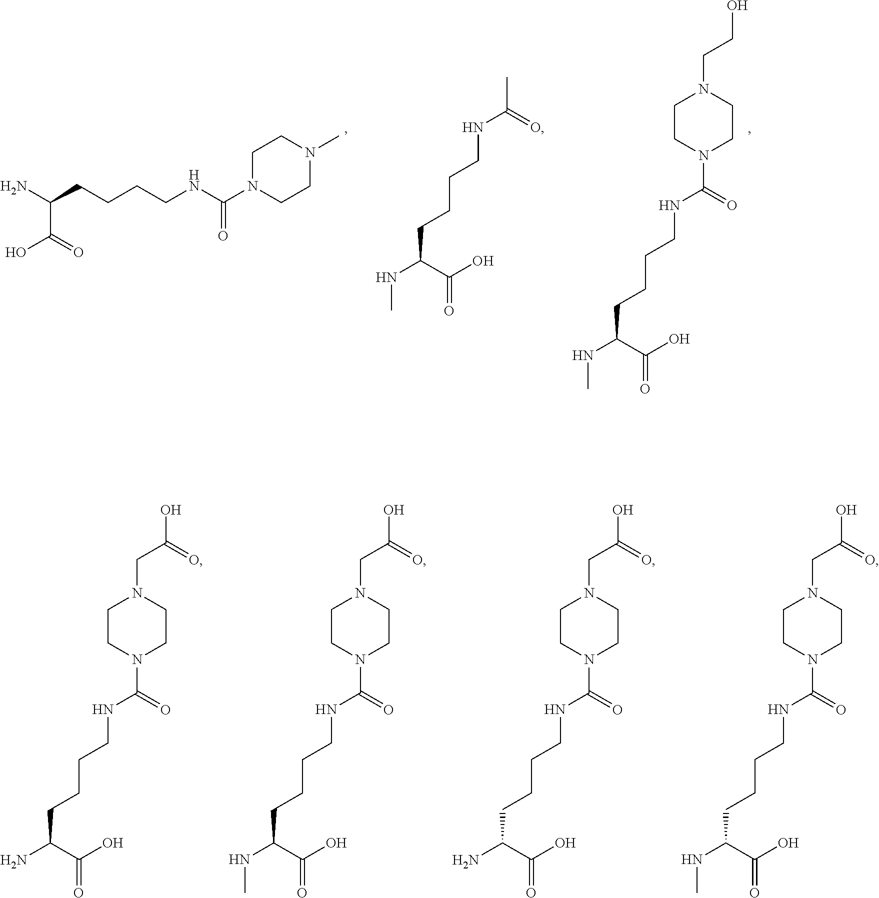

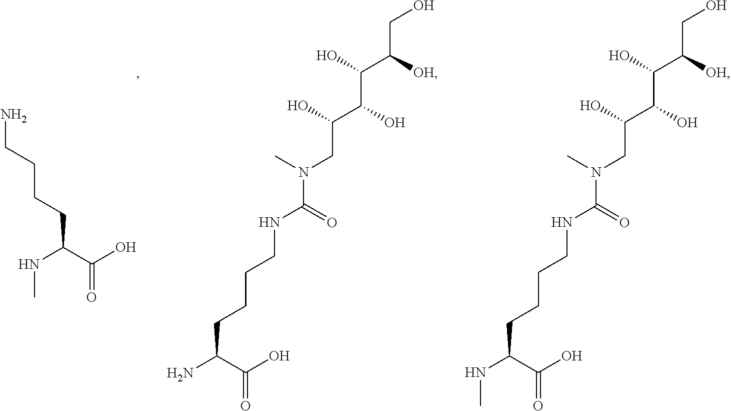 [SMILES: CN[C@@H](CCCCN)C(=O)O ; CN(C[C@H](O)[C@@H](O)[C@H](O)[C@H](O)CO)C(=O)NCCCC[C@H](N)C(=O)O ; CN[C@@H](CCCCNC(=O)N(C)C[C@H](O)[C@@H](O)[C@H](O)[C@H](O)CO)C(=O)O]

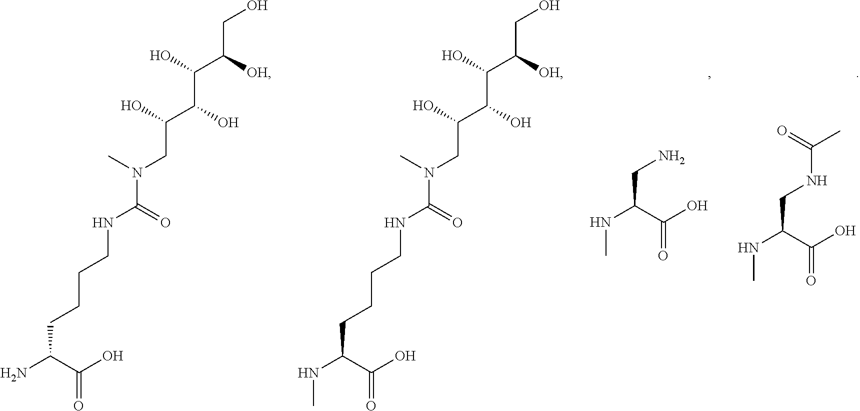

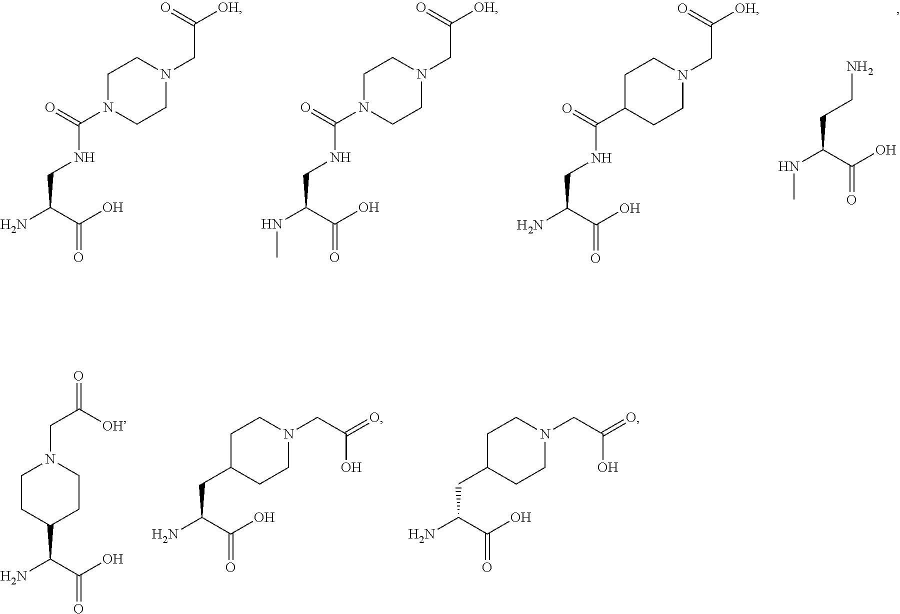

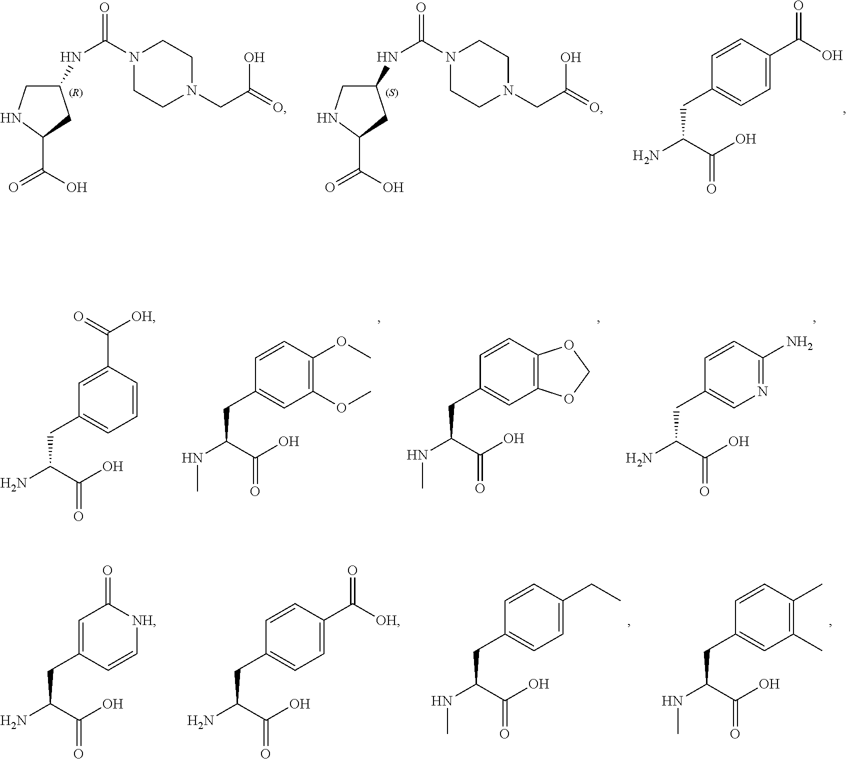

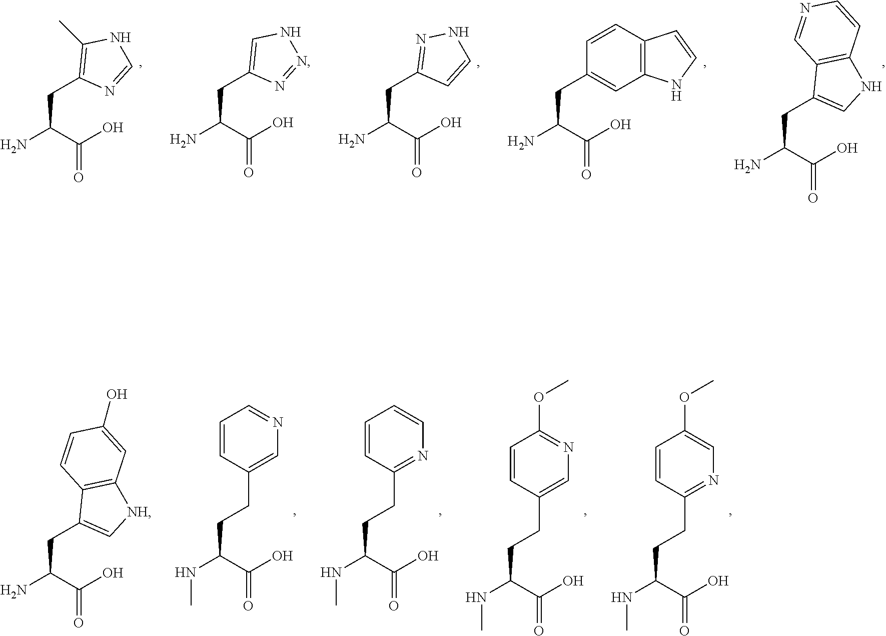

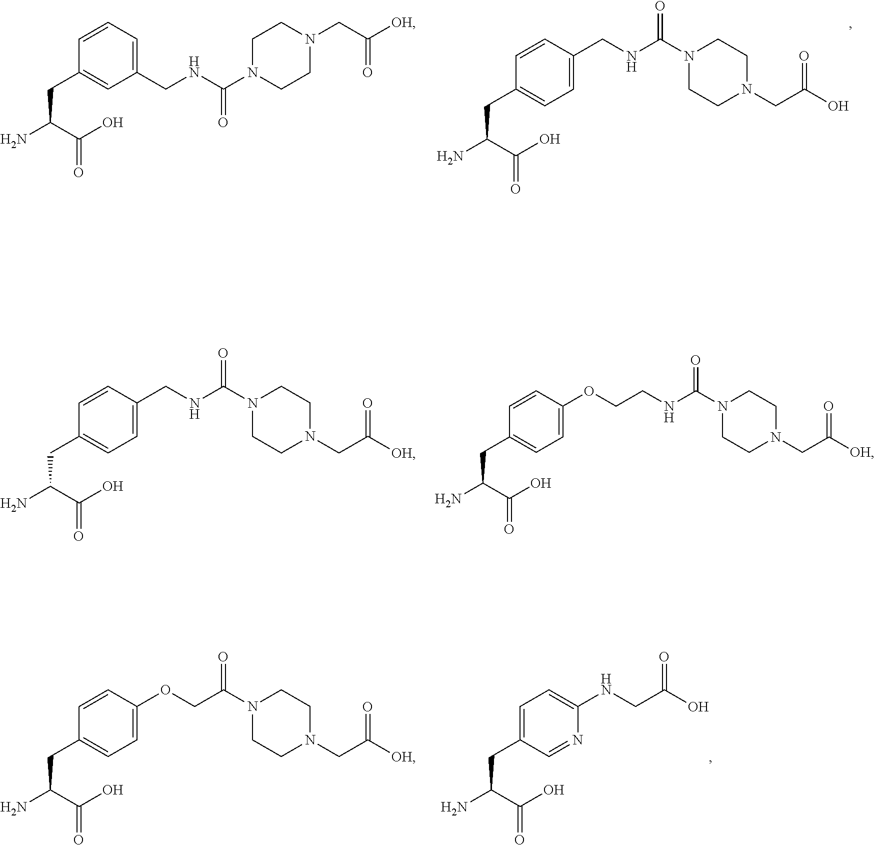 [SMILES: N[C@@H](Cc1cccc(CNC(=O)N2CCN(CC(=O)O)CC2)c1)C(=O)O ; N[C@@H](Cc1ccc(CNC(=O)N2CCN(CC(=O)O)CC2)cc1)C(=O)O ; N[C@H](Cc1ccc(CNC(=O)N2CCN(CC(=O)O)CC2)cc1)C(=O)O ; N[C@@H](Cc1ccc(OCCNC(=O)N2CCN(CC(=O)O)CC2)cc1)C(=O)O ; N[C@@H](Cc1ccc(OCC(=O)N2CCN(CC(=O)O)CC2)cc1)C(=O)O ; N[C@@H](Cc1ccc(NCC(=O)O)nc1)C(=O)O]

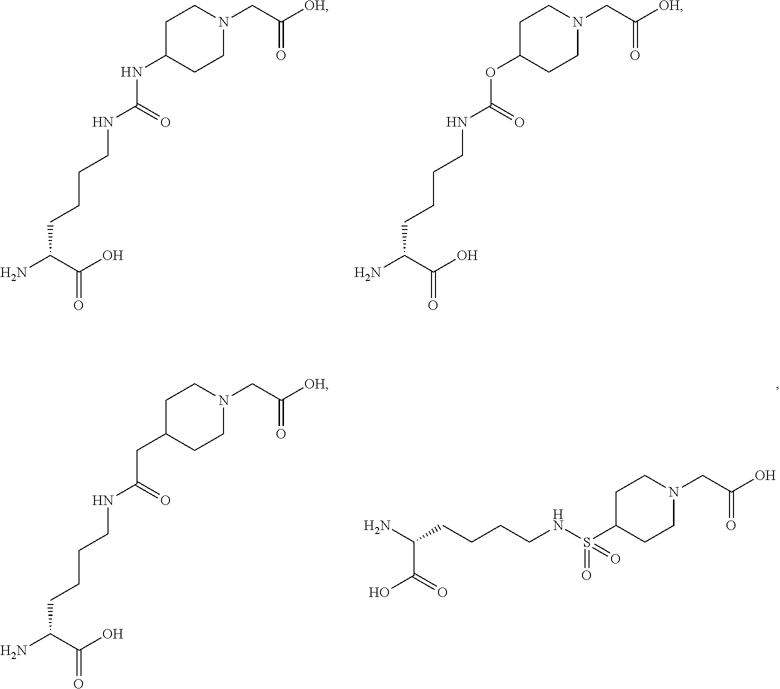 [SMILES: N[C@H](CCCCNC(=O)NC1CCN(CC(=O)O)CC1)C(=O)O ; N[C@H](CCCCNC(=O)OC1CCN(CC(=O)O)CC1)C(=O)O ; N[C@H](CCCCNC(=O)CC1CCN(CC(=O)O)CC1)C(=O)O ; N[C@H](CCCCNS(=O)(=O)C1CCN(CC(=O)O)CC1)C(=O)O]

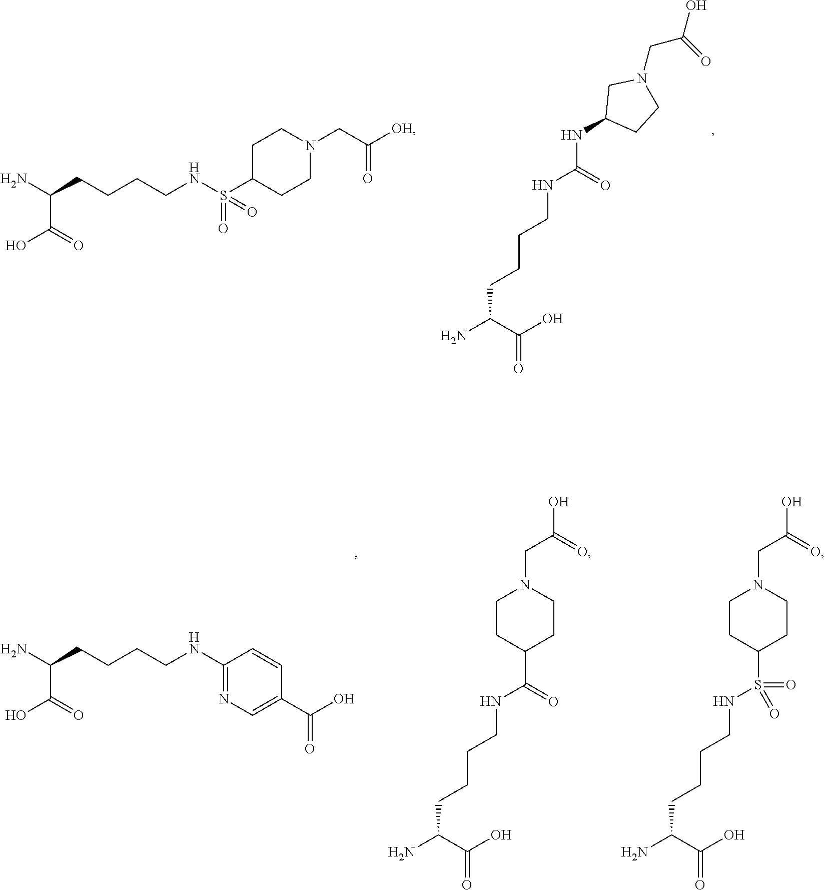 [SMILES: N[C@@H](CCCCNS(=O)(=O)C1CCN(CC(=O)O)CC1)C(=O)O ; N[C@H](CCCCNC(=O)N[C@@H]1CCN(CC(=O)O)C1)C(=O)O ; N[C@@H](CCCCNc1ccc(C(=O)O)cn1)C(=O)O ; N[C@H](CCCCNC(=O)C1CCN(CC(=O)O)CC1)C(=O)O ; N[C@H](CCCCNS(=O)(=O)C1CCN(CC(=O)O)CC1)C(=O)O]

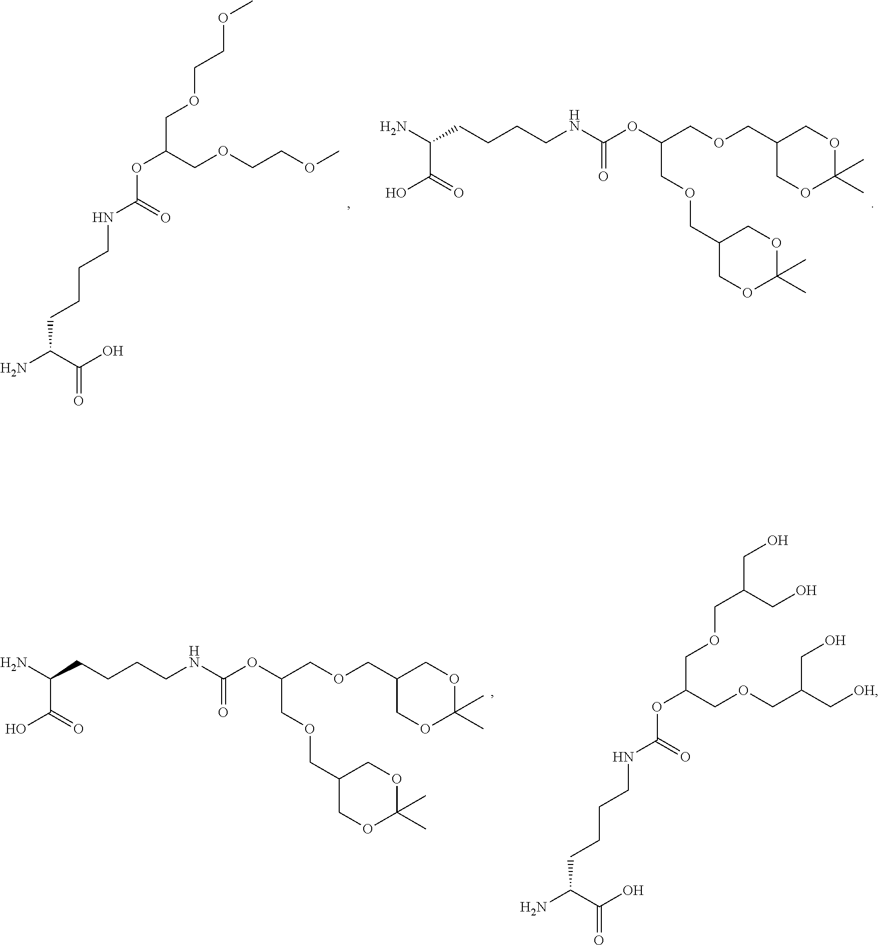 [SMILES: COCCOCC(COCCOC)OC(=O)NCCCC[C@@H](N)C(=O)O ; CC1(C)OCC(COCC(COCC2COC(C)(C)OC2)OC(=O)NCCCC[C@@H](N)C(=O)O)CO1 ; CC1(C)OCC(COCC(COCC2COC(C)(C)OC2)OC(=O)NCCCC[C@H](N)C(=O)O)CO1 ; N[C@H](CCCCNC(=O)OC(COCC(CO)CO)COCC(CO)CO)C(=O)O]

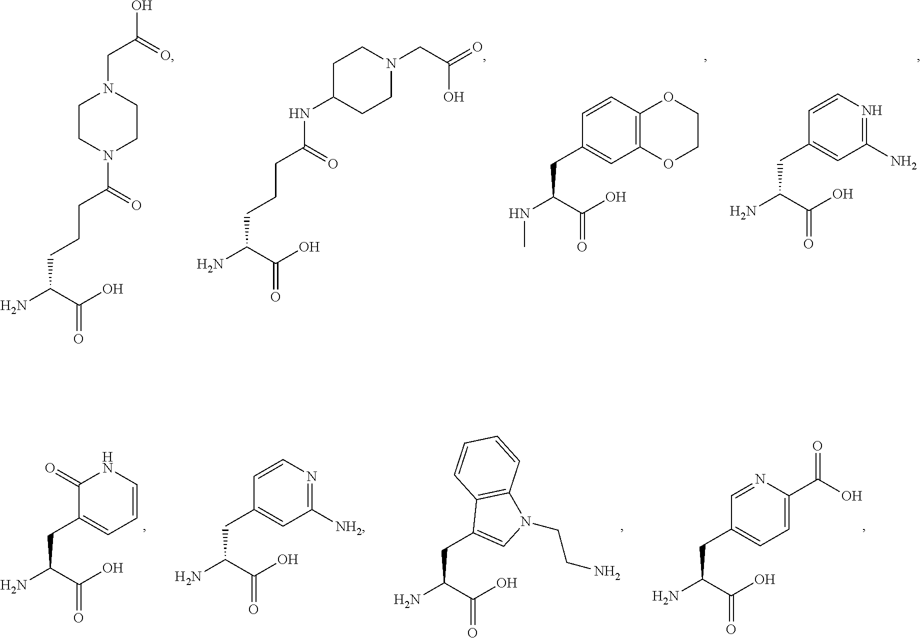

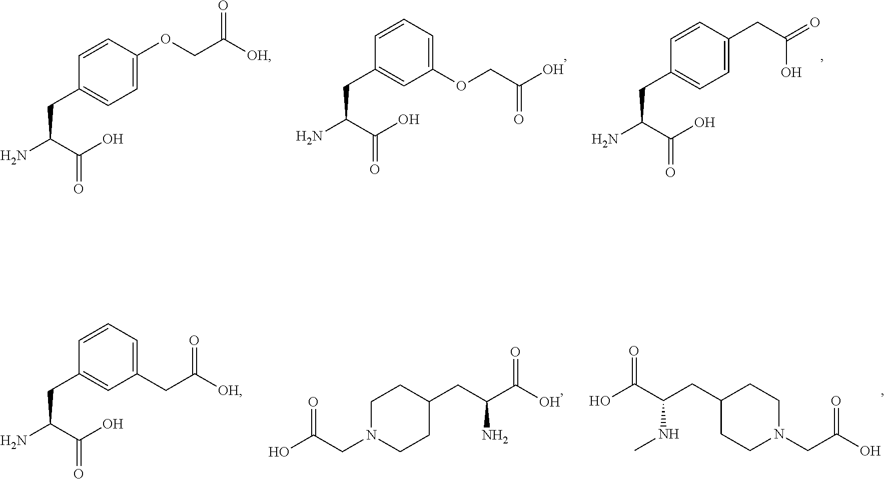 [SMILES: N[C@@H](Cc1ccc(OCC(=O)O)cc1)C(=O)O ; N[C@@H](Cc1cccc(OCC(=O)O)c1)C(=O)O ; N[C@@H](Cc1ccc(CC(=O)O)cc1)C(=O)O ; N[C@@H](Cc1cccc(CC(=O)O)c1)C(=O)O ; N[C@@H](CC1CCN(CC(=O)O)CC1)C(=O)O ; CN[C@@H](CC1CCN(CC(=O)O)CC1)C(=O)O]

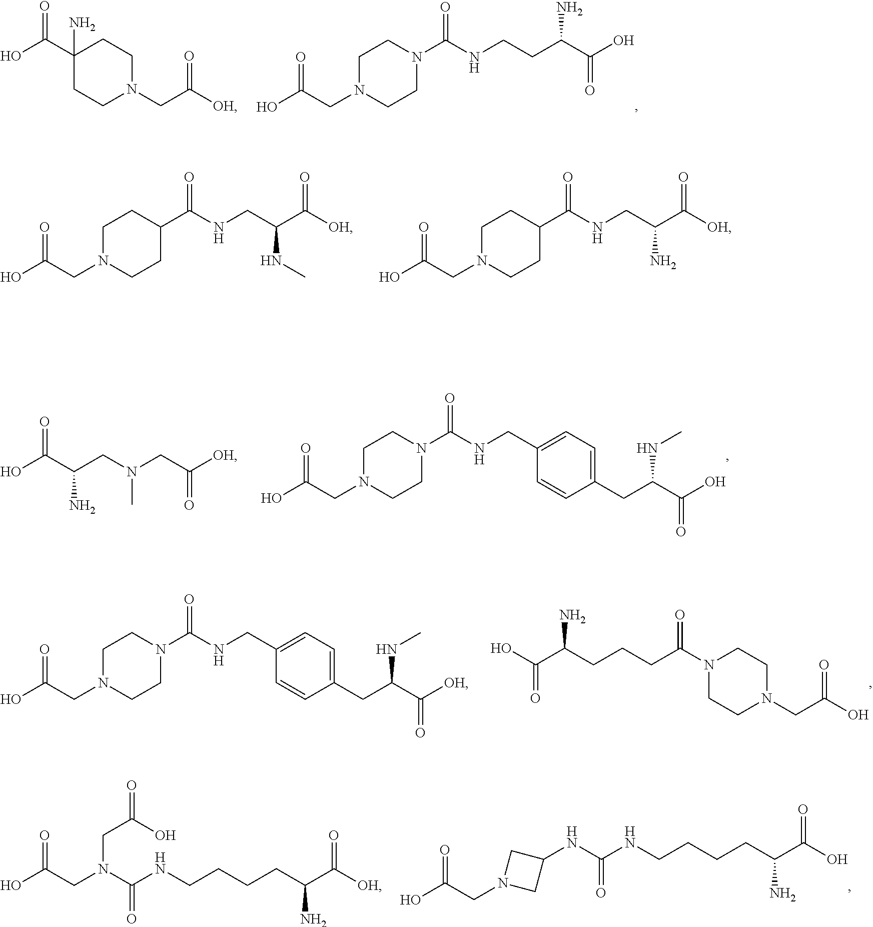

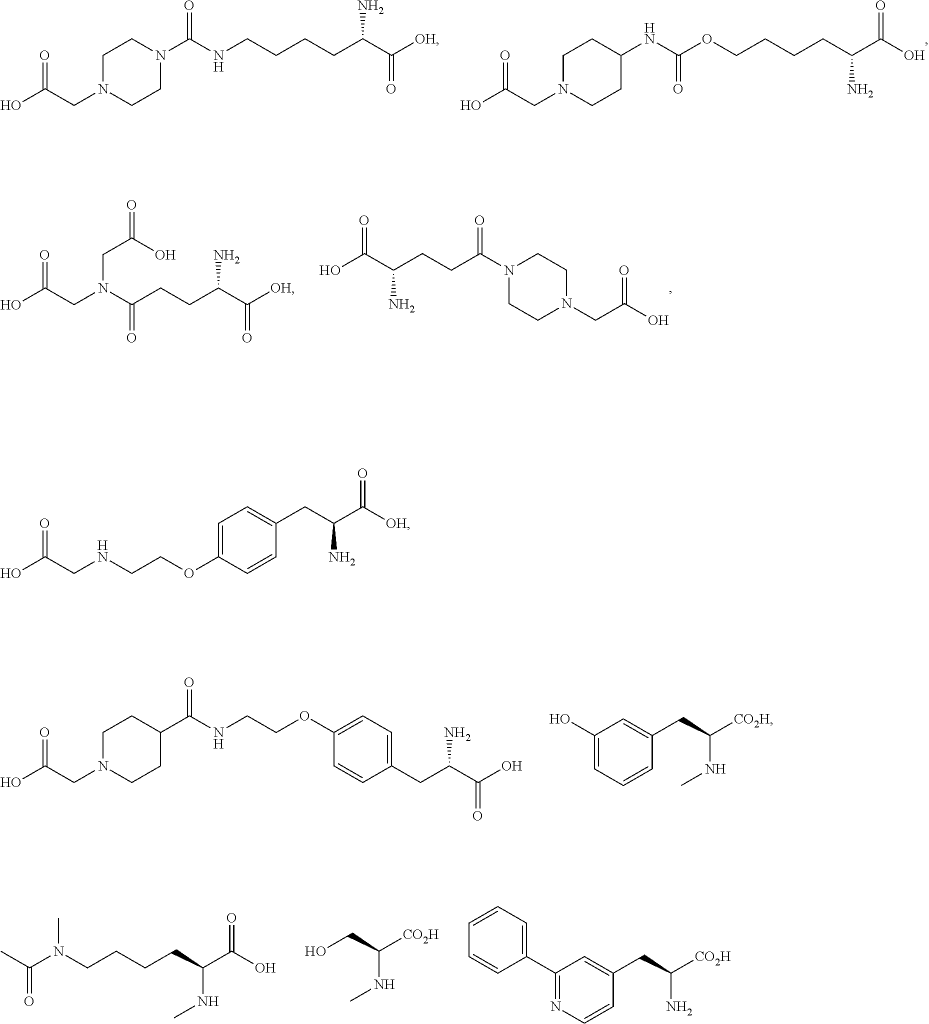

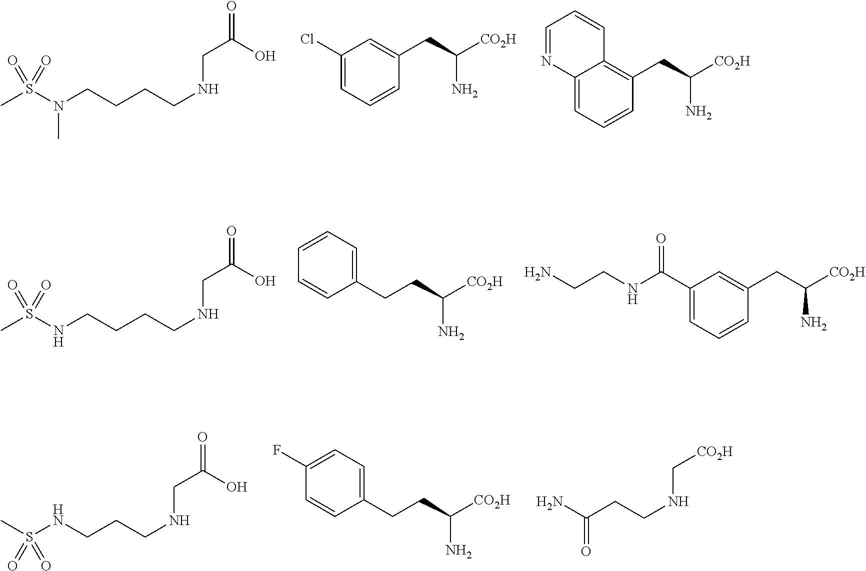

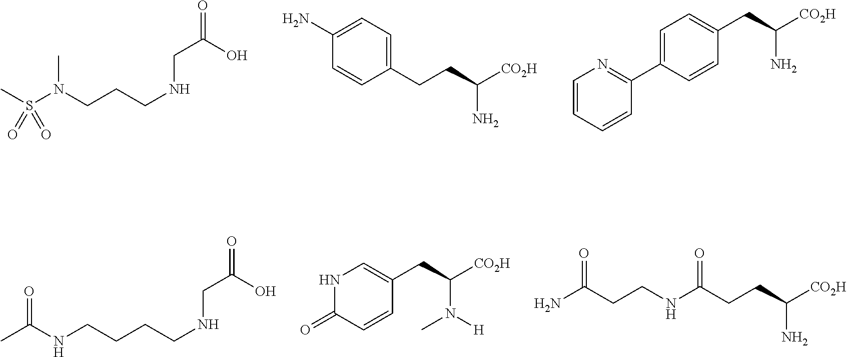 [SMILES: CN(CCCNCC(=O)O)S(C)(=O)=O ; Nc1ccc(CC[C@H](N)C(=O)O)cc1 ; N[C@@H](Cc1ccc(-c2ccccn2)cc1)C(=O)O ; CC(=O)NCCCCNCC(=O)O ; CN[C@@H](Cc1ccc(=O)[nH]c1)C(=O)O ; NC(=O)CCNC(=O)CC[C@H](N)C(=O)O]

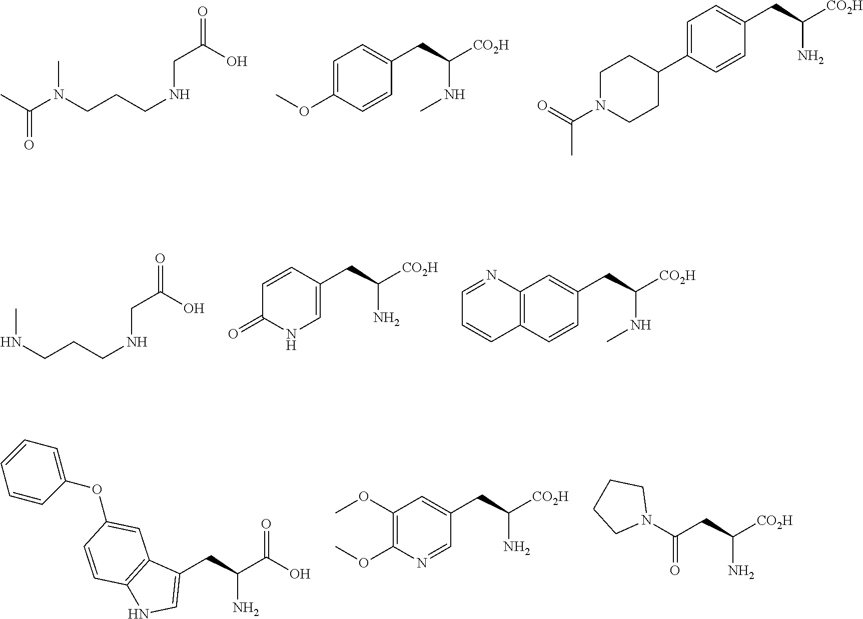

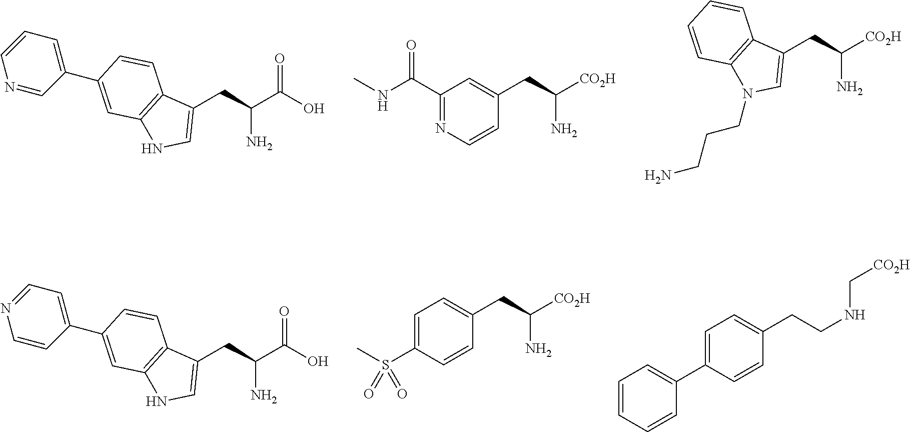

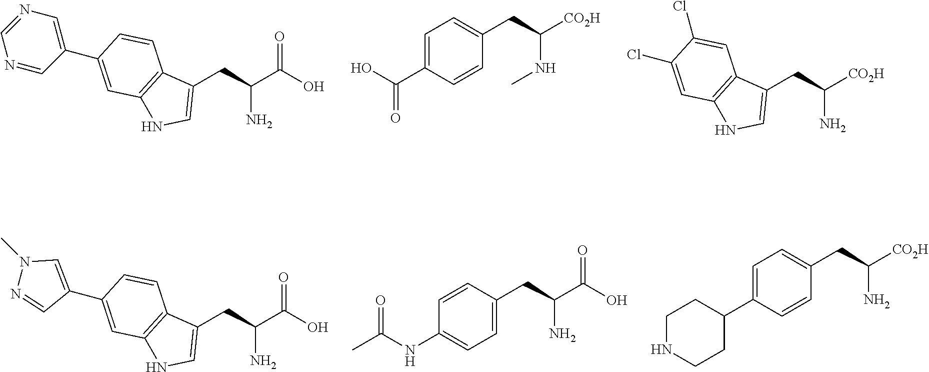

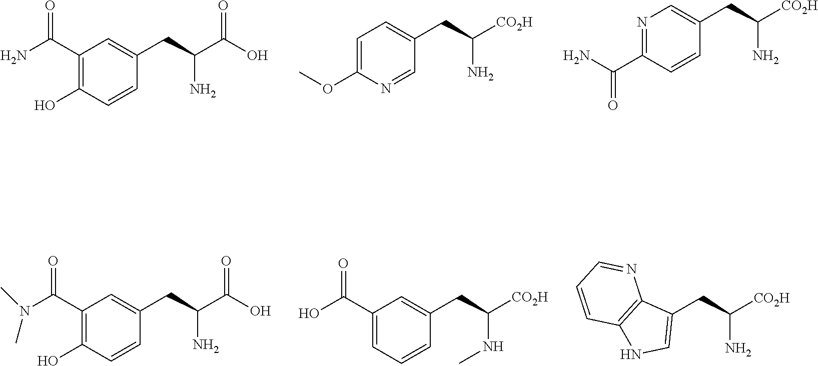 [SMILES: NC(=O)c1cc(C[C@H](N)C(=O)O)ccc1O ; COc1ccc(C[C@H](N)C(=O)O)cn1 ; NC(=O)c1ccc(C[C@H](N)C(=O)O)cn1 ; CN(C)C(=O)c1cc(C[C@H](N)C(=O)O)ccc1O ; CN[C@@H](Cc1cccc(C(=O)O)c1)C(=O)O ; N[C@@H](Cc1c[nH]c2cccnc12)C(=O)O]

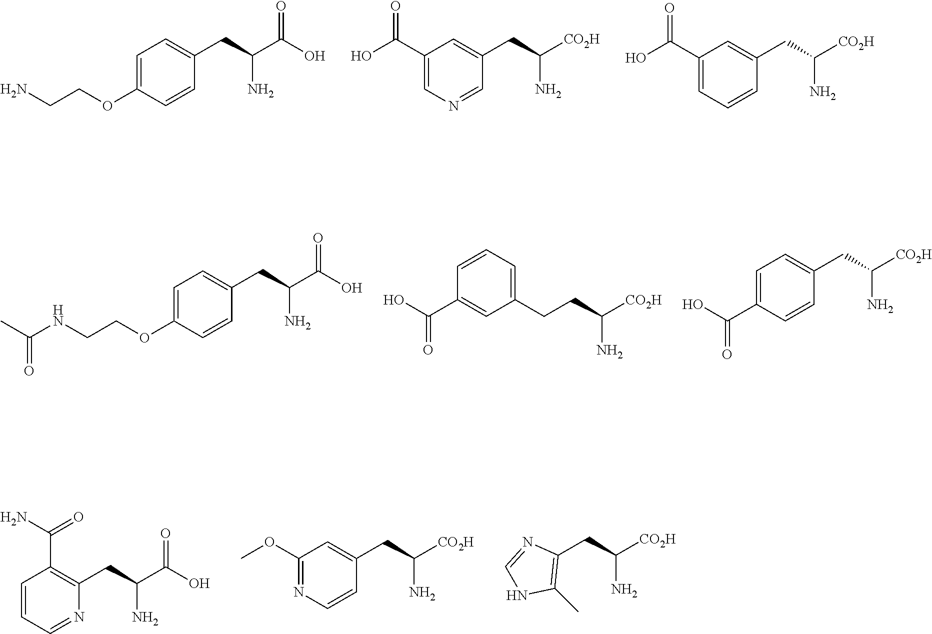

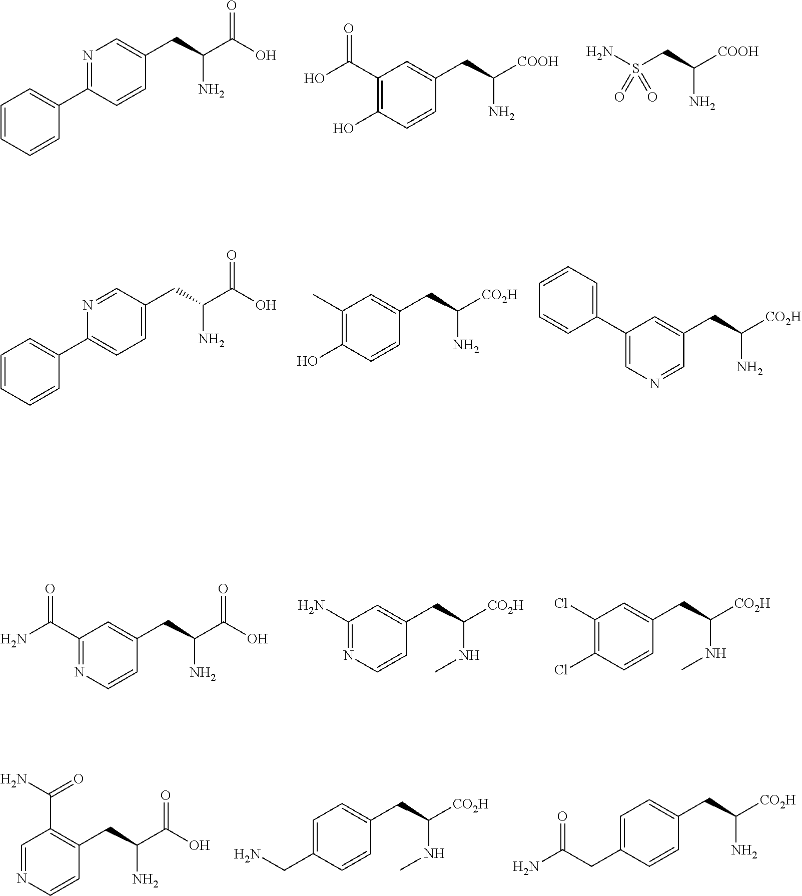

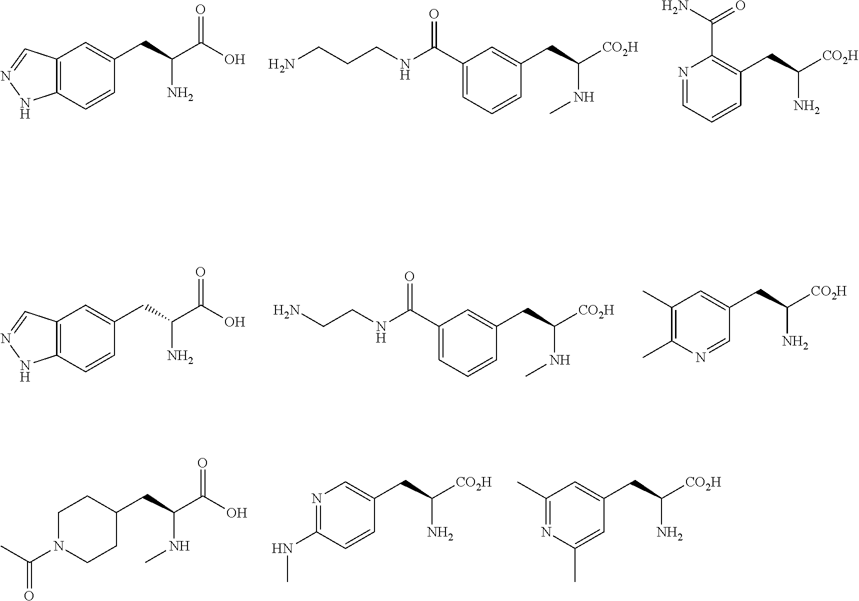

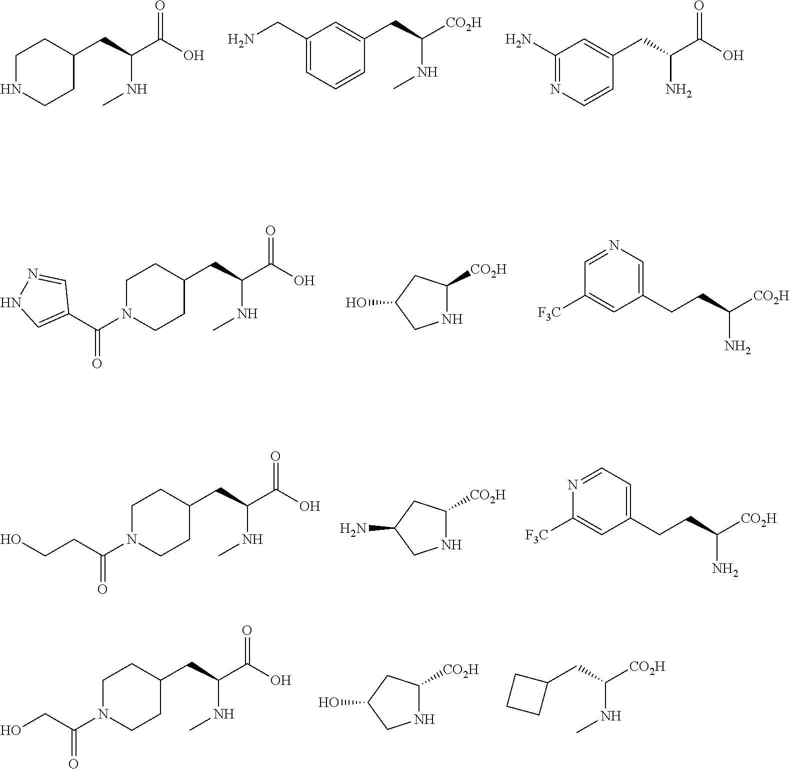

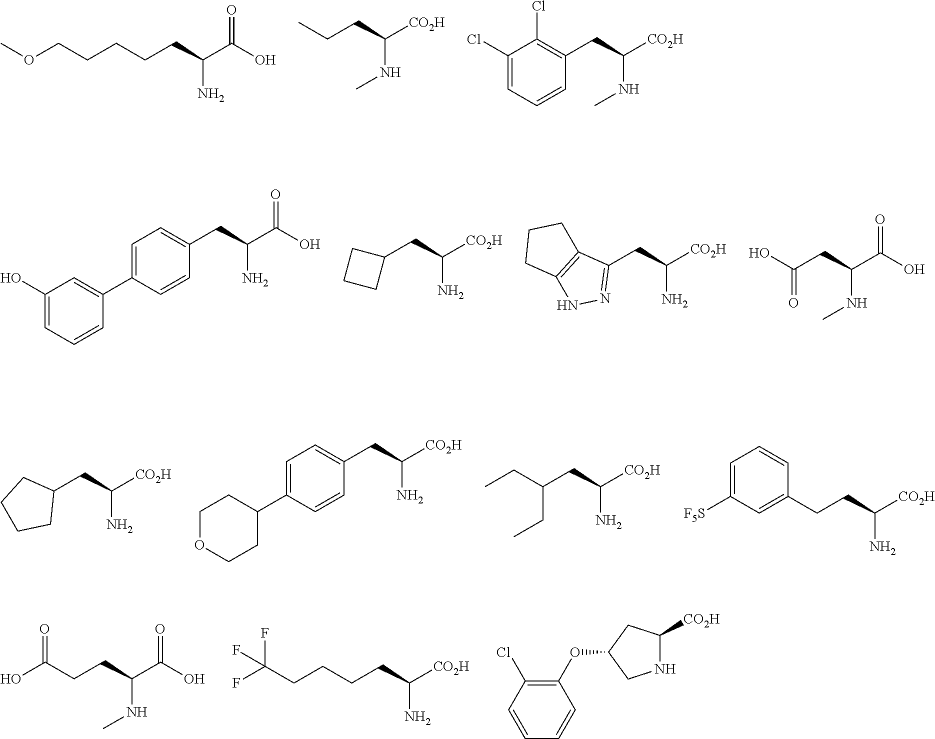

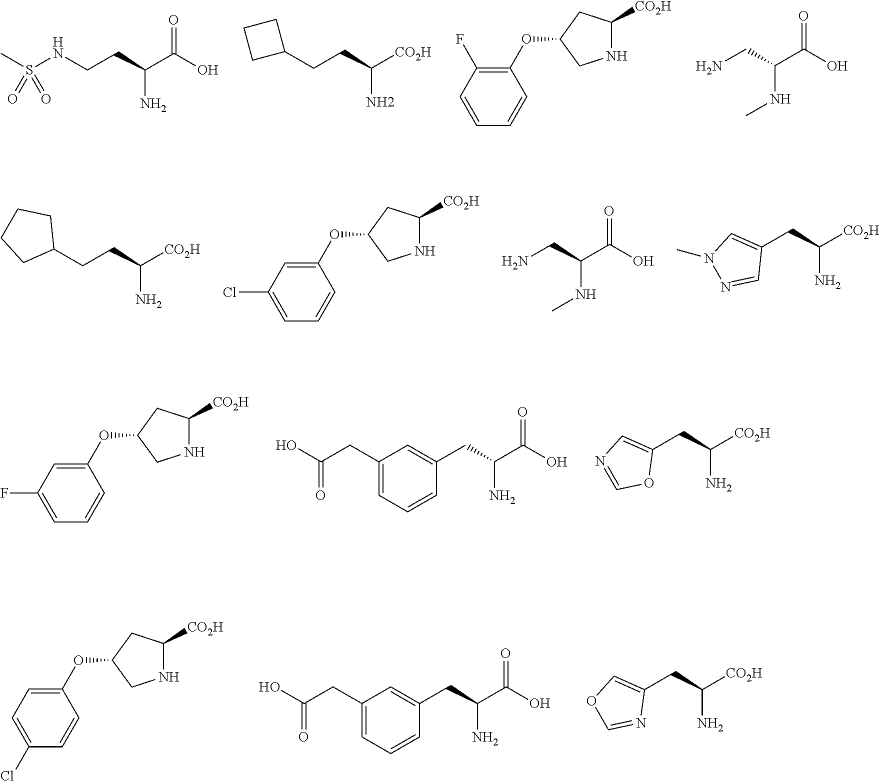

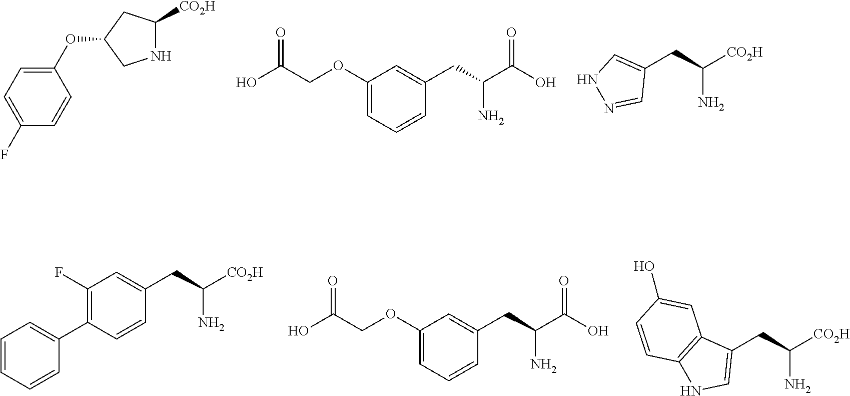

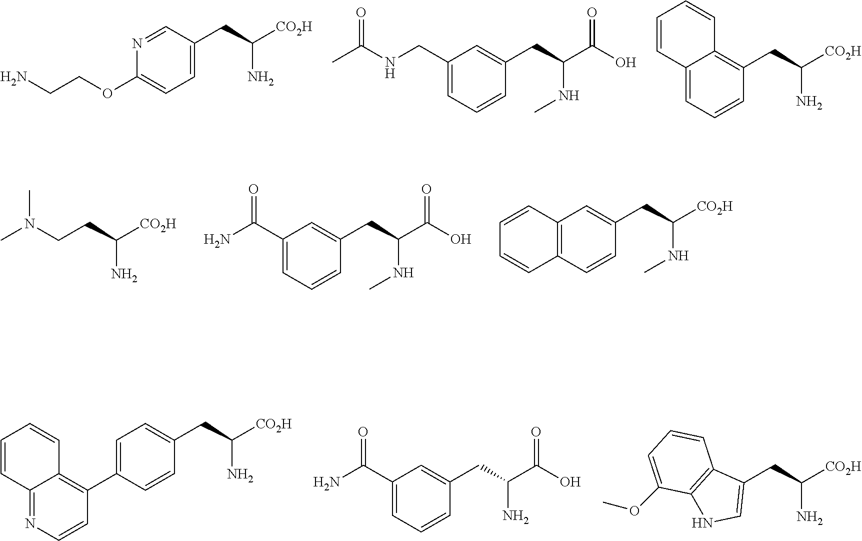

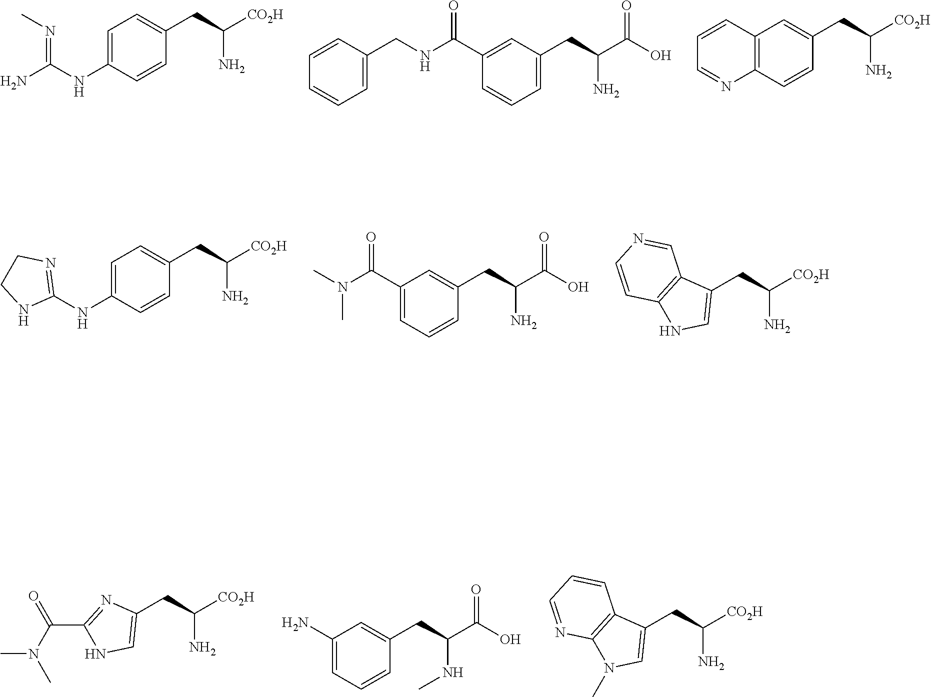

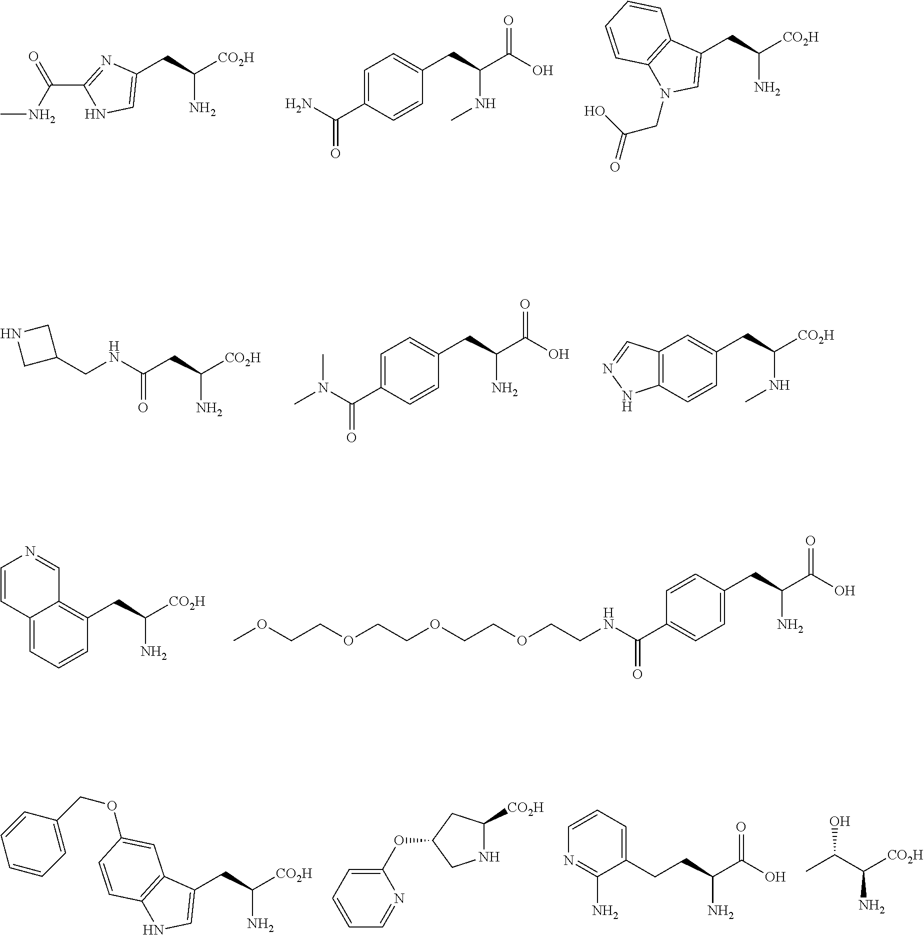

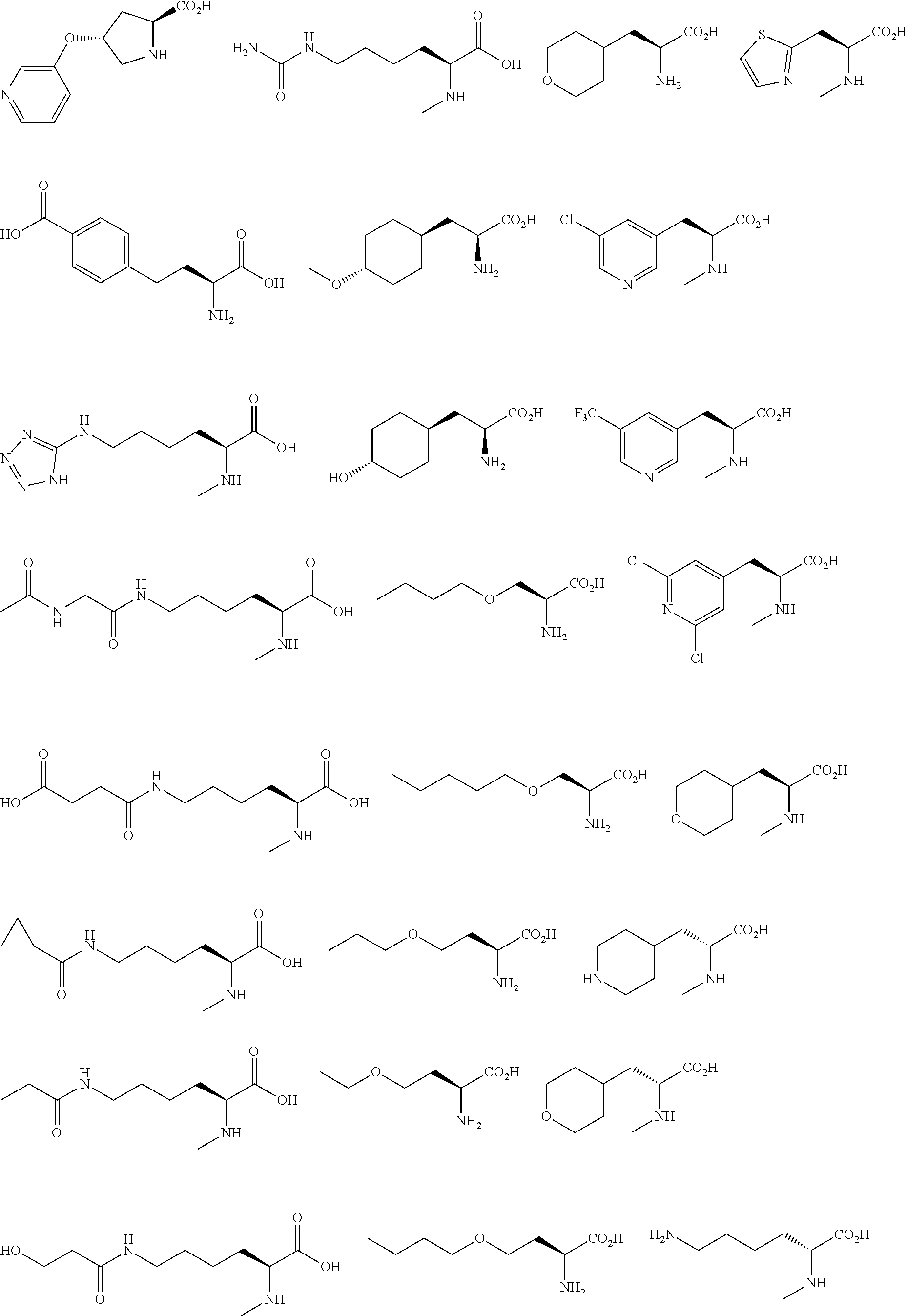

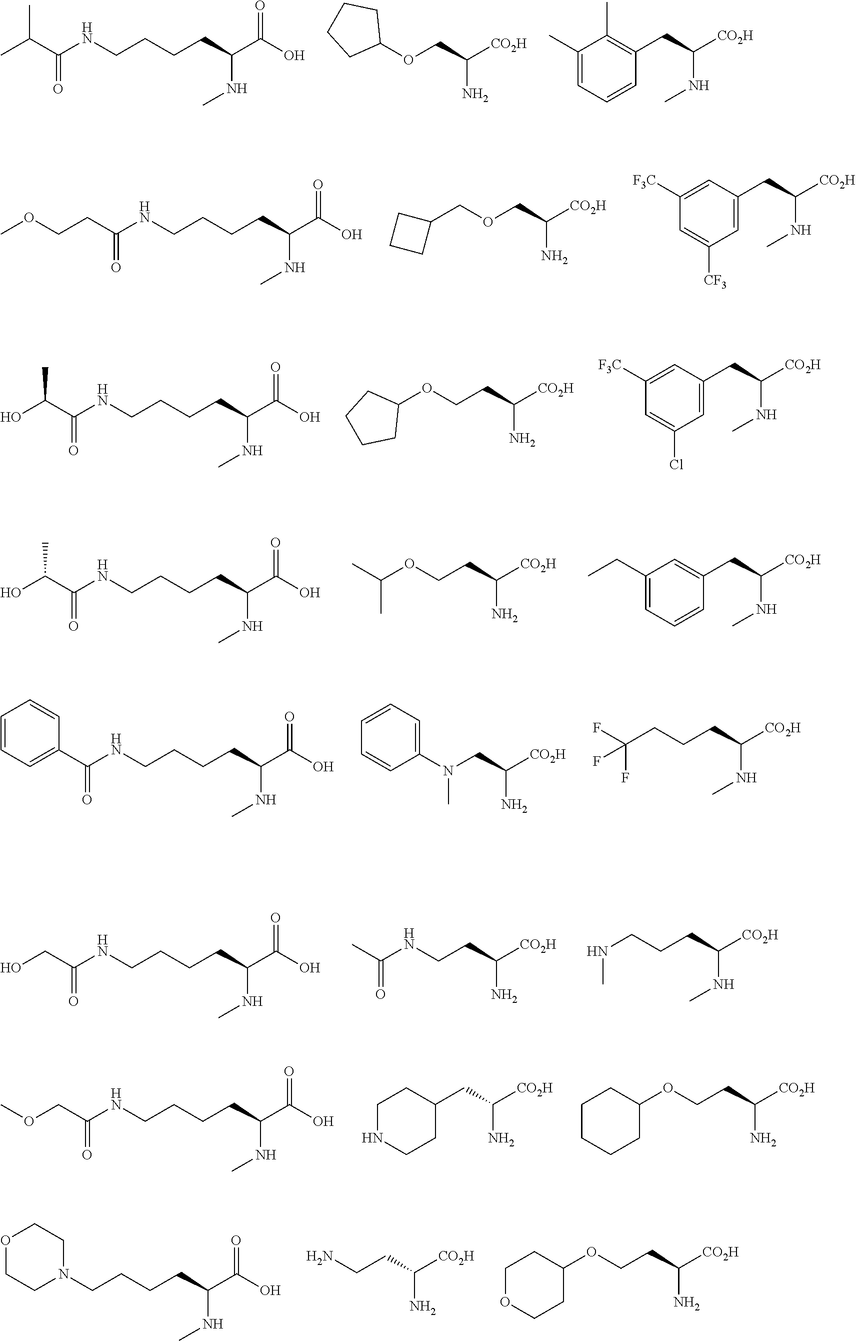

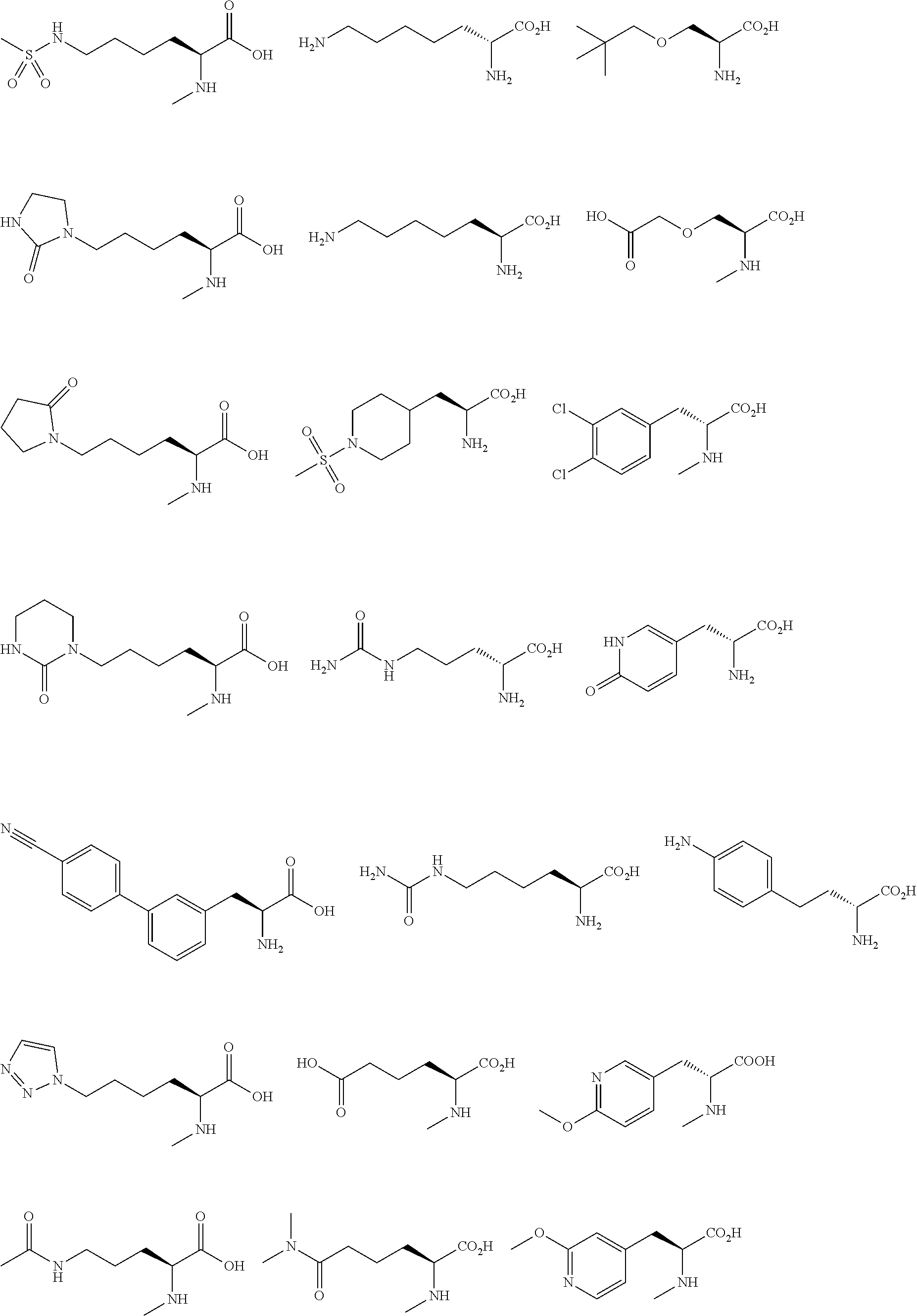

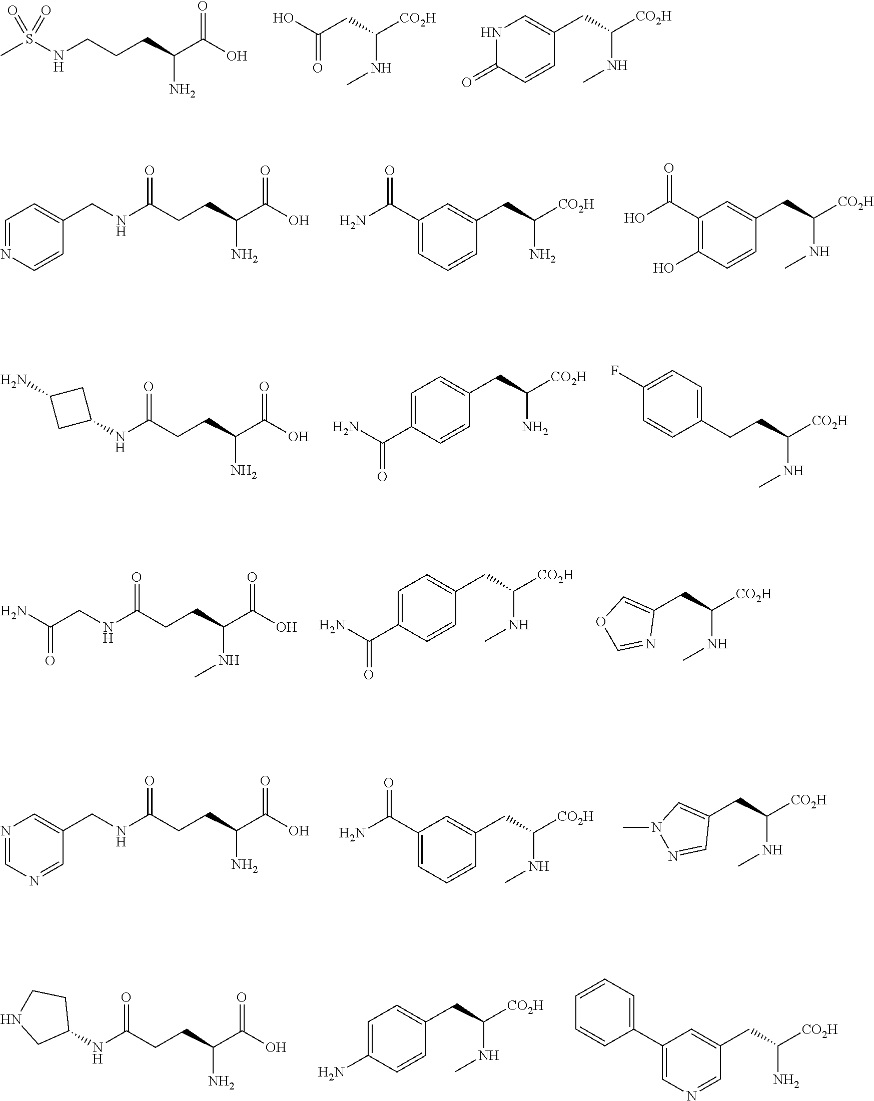

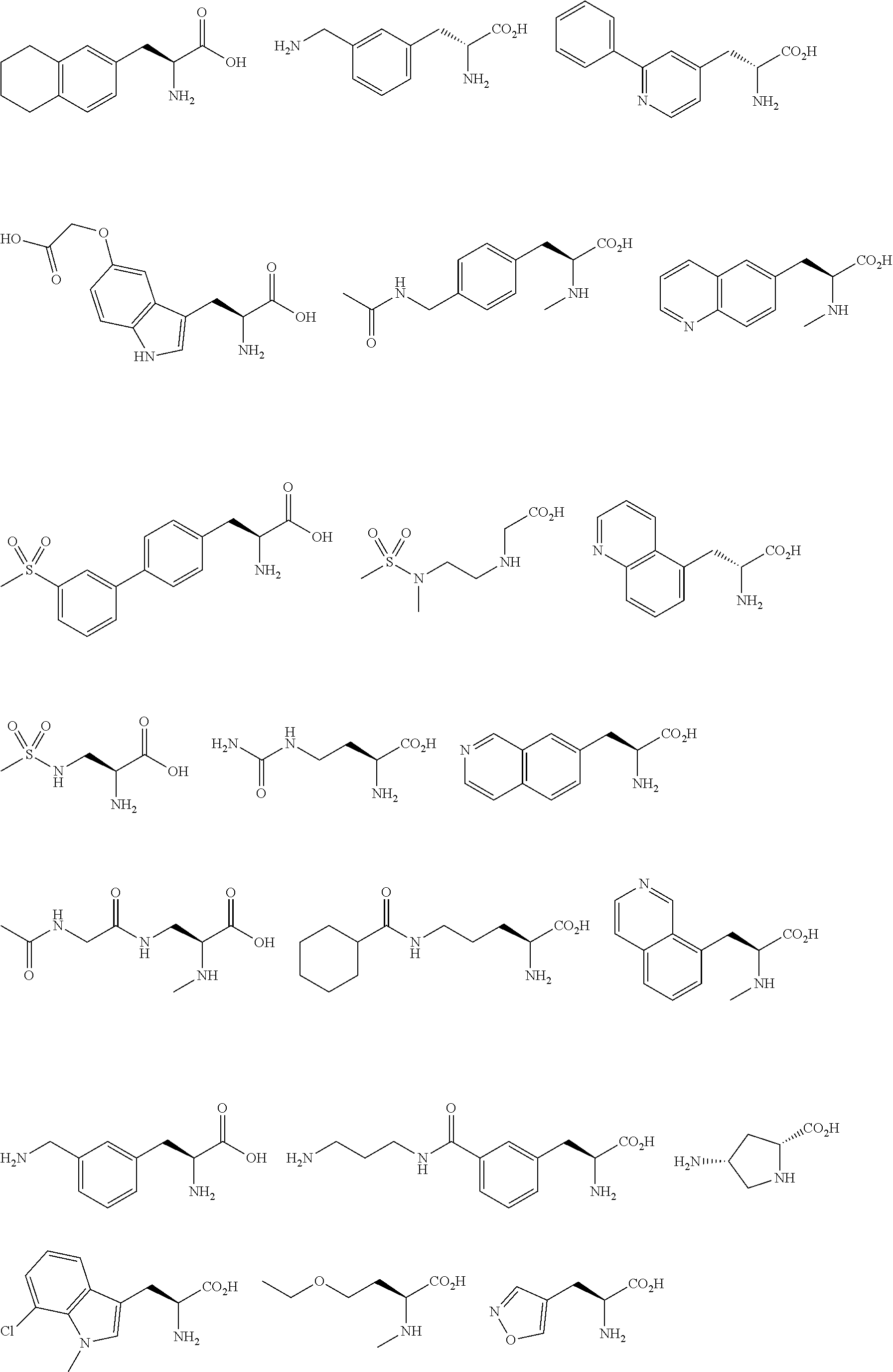

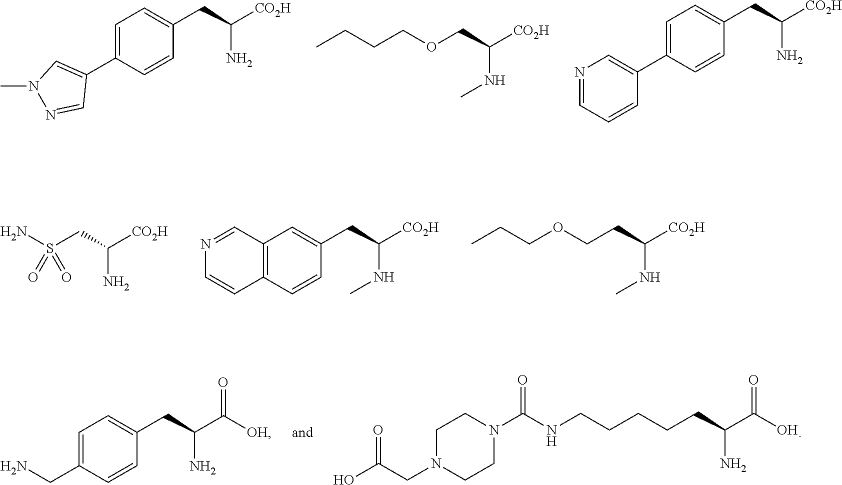

In other words, the novel amino acid derivative of this disclosure is at least one selected from the group consisting of 2-amino-6-[(4-methylpiperazine-1-carbonyl)amino]hexanoic acid, 6-acetamido-2-(methylamino)hexanoic acid, 2-amino-6-{[4-(2-hydroxyethyl)piperazine-1-carbonyl]amino}hexanoic acid, 2-amino-6-{[4-(carboxymethyl)piperazine-1-carbonyl]amino}hexanoic acid, 6-{[4-(carboxymethyl)piperazine-1-carbonyl]amino}-2-(methylamino)hexanoic acid, 2-amino-6-{[4-(carboxymethyl)piperazine-1-carbonyl]amino}hexanoic acid, 6-{[4-(carboxymethyl)piperazine-1-carbonyl]amino}-2-(methylamino)hexanoic acid, 6-amino-2-(methylamino)hexanoic acid, 2-amino-6-({methyl[(2S,3R,4R,5R)-2,3,4,5,6-pentahydroxyhexyl]carbamoyl}amino)hexanoic acid, 6-({methyl[(2S,3R,4R,5R)-2,3,4,5,6-pentahydroxyhexyl]carbamoyl}amino)-2-(methylamino)hexanoic acid, 2-amino-6-({methyl[(2S,3R,4R,5R)-2,3,4,5,6-pentahydroxyhexyl]carbamoyl}amino)hexanoic acid, 6-({methyl[(2S,3R,4R,5R)-2,3,4,5,6-pentahydroxyhexyl]carbamoyl}amino)-2-(methylamino)hexanoic acid, 3-amino-2-(methylamino)propanoic acid, 3-acetamido-2-(methylamino)propanoic acid, 2-amino-3-{[4-(carboxymethyl)piperazine-1-carbonyl]amino}propanoic acid, 3-{[4-(carboxymethyl)piperazine-1-carbonyl]amino}-2-(methylamino)propanoic acid, 2-amino-3-{[1-(carboxymethyl)piperidin-4-yl]formamido}propanoic acid, 4-amino-2-(methylamino)butanoic acid, 2-amino-2-[1-(carboxymethyl)piperidin-4-yl]acetic acid, 2-amino-3-[1-(carboxymethyl)piperidin-4-yl]propanoic acid, 2-amino-3-[1-(carboxymethyl)piperidin-4-yl]propanoic acid, (4R)-4-{[4-(carboxymethyl)piperazine-1-carbonyl]amino}pyrrolidine-2-carboxylic acid, (4S)-4-{[4-(carboxymethyl)piperazine-1-carbonyl]amino}pyrrolidine-2-carboxylic acid, 4-(2-amino-2-carboxyethyl)benzoic acid, 3-(2-amino-2-carboxyethyl)benzoic acid, 3-(3,4-dimethoxyphenyl)-2-(methylamino)propanoic acid, 3-(2H-1,3-benzodioxol-5-yl)-2-(methylamino)propanoic acid, 2-amino-3-(6-aminopyridin-3-yl)propanoic acid, 2-amino-3-(2-oxo-1,2-dihydropyridin-4-yl)propanoic acid, 4-(2-amino-2-carboxyethyl)benzoic acid, 3-(4-ethylphenyl)-2-(methylamino)propanoic acid, 3-(3,4-dimethylphenyl)-2-(methylamino)propanoic acid, 2-amino-3-(5-methyl-1H-imidazol-4-yl)propanoic acid, 2-amino-3-(1H-1,2,3-triazol-4-yl)propanoic acid, 2-amino-3-(1H-pyrazol-3-yl)propanoic acid, 2-amino-3-(1H-indol-6-yl)propanoic acid, 2-amino-3-{1H-pyrrolo[3,2-c]pyridin-3-yl}propanoic acid, 2-amino-3-(6-hydroxy-1H-indol-3-yl)propanoic acid, 2-(methylamino)-4-(pyridin-3-yl)butanoic acid, 2-(methylamino)-4-(pyridin-2-yl)butanoic acid, 4-(6-methoxypyridin-3-yl)-2-(methylamino)butanoic acid, 4-(5-methoxypyridin-2-yl)-2-(methylamino)butanoic acid, 2-amino-3-[3-({[4-(carboxy methyl)piperazine-1-carbonyl]amino}methyl)phenyl]propanoic acid, 2-amino-3-[4-({[4-(carboxymethyl)piperazine-1-carbonyl]amino}methyl)phenyl]propanoic acid, 2-amino-3-[4-({[4-(carboxymethyl)piperazine-1-carbonyl]amino}methyl)phenyl]propanoic acid, 2-amino-3-[4-(2-{[4-(carboxymethyl)piperazine-1-carbonyl]amino}ethoxy)phenyl]propanoic acid, 2-amino-3-(4-{2-[4-(carboxymethyl)piperazin-1-yl]-2-oxoethoxy}phenyl)propanoic acid, 2-amino-3-{6-[(carboxymethyl)amino]pyridin-3-yl}propanoic acid, 2-amino-6-({[1-(carboxymethyl)piperidin-4-yl]carbamoyl}amino)hexanoic acid, 2-amino-6-[({[1-(carboxymethyl)piperidin-4-yl]oxy}carbonyl)amino]hexanoic acid, 2-amino-6-{2-[1-(carboxymethyl)piperidin-4-yl]acetamido}hexanoic acid, 2-amino-6-[1-(carboxymethyl)piperidine-4-sulfonamido]hexanoic acid, 2-amino-6-[1-(carboxymethyl)piperidine-4-sulfonamido]hexanoic acid, 2-amino-6-({[(3R)-1-(carboxymethyl)pyrrolidin-3-yl]carbamoyl}amino)hexanoic acid, 6-[(5-amino-5-carboxypentyl)amino]pyridine-3-carboxylic acid, 2-amino-6-{[1-(carboxymethyl)piperidin-4-yl]formamido}hexanoic acid, 2-amino-6-[1-(carboxymethyl)piperidine-4-sulfonamido]hexanoic acid, 2-amino-6-{4-[(2-methoxyethoxy)methyl]-2-oxo-3,6,9-trioxa-1-azadecan-1-yl}hexanoic acid, 2-amino-6-{[({1,3-bis[(2,2-dimethyl-1,3-dioxan-5-yl)methoxy]propan-2-yl}oxy)carbonyl]amino}hexanoic acid, 2-amino-6-{[({1,3-bis[(2,2-dimethyl-1,3-dioxan-5-yl)methoxy]propan-2-yl}oxy)carbonyl]amino}hexanoic acid, 2-amino-6-{[({1,3-bis[3-hydroxy-2-(hydroxymethyl)propoxy]propan-2-yl}oxy)carbonyl]amino}hexanoic acid, 2-amino-6-[4-(carboxymethyl)piperazin-1-yl]-6-oxohexanoic acid, 2-amino-5-{[1-(carboxymethyl)piperidin-4-yl]carbamoyl}pentanoic acid, 3-(2,3-dihydro-1,4-benzodioxin-6-yl)-2-(methylamino)propanoic acid, 2-amino-3-(2-aminopyridin-4-yl)propanoic acid, 2-amino-3-(2-oxo-1,2-dihydropyridin-3-yl)propanoic acid, 2-amino-3-(2-aminopyridin-4-yl)propanoic acid, 2-amino-3-[1-(2-aminoethyl)-1H-indol-3-yl]propanoic acid, 5-(2-amino-2-carboxyethyl)pyridine-2-carboxylic acid, 2-amino-3-[4-(carboxymethoxy)phenyl]propanoic acid, 2-amino-3-[3-(carboxymethoxy)phenyl]propanoic acid, 2-amino-3-[4-(carboxymethyl)phenyl]propanoic acid, 2-amino-3-[3-(carboxymethyl)phenyl]propanoic acid, (2S)-2-amino-6-{[bis(carboxymethyl)carbamoyl]amino}hexanoic acid, (2S)-2-amino-6-{[4-(carboxymethyl)piperazine-1-carbonyl]amino}hexanoic acid, (2S)-2-amino-5-[4-(carboxymethyl)piperazin-1-yl]-5-oxopentanoic acid, (2R)-2-amino-3-{[1-(carboxymethyl)piperidin-4-yl]formamido}propanoic acid, (2S)-3-{[1-(carboxymethyl)piperidin-4-yl]formamido}-2-(methylamino)propanoic acid, (2S)-2-amino-6-[4-(carboxy methyl)piperazin-1-yl]-6-oxohexanoic acid, (2S)-2-amino-4-[bis(carboxymethyl)carbamoyl]butanoic acid, (2S)-2-amino-3-[1-(carboxymethyl)piperidin-4-yl]propanoic acid, 4-amino-1-(carboxymethyl)piperidine-4-carboxylic acid, (2S)-3-[1-(carboxymethyl)piperidin-4-yl]-2-(methylamino)propanoic acid, (2S)-3-[4-({[4-(carboxymethyl)piperazine-1-carbonyl]amino}methyl)phenyl]-2-(methylamino)propanoic acid, (2R)-3-[4-({[4-(carboxymethyl)piperazine-1-carbonyl]amino}methyl)phenyl]-2-(methylamino)propanoic acid, (2S)-3-(3-hydroxyphenyl)-2-(methylamino)propanoic acid, (2S)-2-amino-3-[4-(2-{[1-(carboxymethyl)piperidin-4-yl]formamido}ethoxy)phenyl]propanoic acid, (2S)-2-amino-3-(4-{2-[(carboxymethyl)amino]ethoxy}phenyl)propanoic acid, (2R)-2-amino-6-({[1-(carboxymethyl)azetidin-3-yl]carbamoyl}amino)hexanoic acid, (2R)-2-amino-6-({[1-(carboxymethyl)piperidin-4-yl]carbamoyl}oxy)hexanoic acid, (2S)-2-amino-3-[(carboxymethyl)(methyl)amino]propanoic acid, (2S)-2-amino-4-{[4-(carboxymethyl)piperazine-1-carbonyl]amino}butanoic acid, (2S)-2-(methylamino)-6-(N-methylacetamido)hexanoic acid, 2-{[4-(N-methylmethanesulfonamido)butyl]amino}acetic acid, 2-[(4-methanesulfonamidobutyl)amino]acetic acid, 2-[(3-methanesulfonamidopropyl)amino]acetic acid, 2-{[3-(N-methylmethanesulfonamido)propyl]amino}acetic acid, 2-[(4-acetamidobutyl)amino]acetic acid, 2-{[3-(N-methylacetamido)propyl]amino}acetic acid, 2-{[3-(methylamino)propyl]amino}acetic acid, (2S)-2-amino-3-(5-phenoxy-1H-indol-3-yl)propanoic acid, (2S)-2-amino-3-[6-(pyridin-3-yl)-1H-indol-3-yl]propanoic acid, (2S)-2-amino-3-[6-(pyridin-4-yl)-1H-indol-3-yl]propanoic acid, (2S)-2-amino-3-[6-(pyrimidin-5-yl)-1H-indol-3-yl]propanoic acid, (2S)-2-amino-3-[6-(1-methyl-1H-pyrazol-4-yl)-1H-indol-3-yl]propanoic acid, (2S)-2-amino-3-(3-carbamoyl-4-hydroxyphenyl)propanoic acid, (2S)-2-amino-3-[3-(dimethylcarbamoyl)-4-hydroxyphenyl]propanoic acid, (2S)-2-amino-3-[4-(2-aminoethoxy)phenyl]propanoic acid, (2S)-2-amino-3-[4-(2-acetamidoethoxy)phenyl]propanoic acid, (2S)-2-amino-3-(3-carbamoylpyridin-2-yl)propanoic acid, (2S)-2-amino-3-(6-phenylpyridin-3-yl)propanoic acid, (2R)-2-amino-3-(6-phenylpyridin-3-yl)propanoic acid, (2S)-2-amino-3-(2-carbamoylpyridin-4-yl)propanoic acid, (2S)-2-amino-3-(3-carbamoylpyridin-4-yl)propanoic acid, (2S)-2-amino-3-(1H-indazol-5-yl)propanoic acid, (2R)-2-amino-3-(1H-indazol-5-yl)propanoic acid, (2S)-3-(1-acetylpiperidin-4-yl)-2-(methylamino)propanoic acid, (2S)-2-(methylamino)-3-(piperidin-4-yl)propanoic acid, (2S)-2-(methylamino)-3-[1-(1H-pyrazole-4-carbonyl)piperidin-4-yl]propanoic acid, (2S)-3-[1-(3-hydroxypropanoyl)piperidin-4-yl]-2-(methylamino)propanoic acid, (2S)-3-[1-(2-hydroxyacetyl)piperidin-4-yl]-2-(methylamino)propanoic acid, (2S)-2-amino-7-methoxyheptanoic acid, (2S)-2-amino-3-{3′-hydroxy-[1,1′-biphenyl]-4-yl}propanoic acid, (2S)-2-(methylamino)butanedioic acid, (2S)-2-(methylamino)pentanedioic acid, (2S)-2-amino-4-methanesulfonamidobutanoic acid, (2R)-3-amino-2-(methylamino)propanoic acid, (2S)-3-amino-2-(methylamino)propanoic acid, (2R)-2-amino-3-[3-(carboxymethyl)phenyl]propanoic acid, (2S)-2-amino-3-[3-(carboxymethyl)phenyl]propanoic acid, (2R)-2-amino-3-[3-(carboxymethoxy)phenyl]propanoic acid, (2S)-2-amino-3-[3-(carboxymethoxy)phenyl]propanoic acid, (2S)-3-[3-(acetamidomethyl)phenyl]-2-(methylamino)propanoic acid, (2S)-3-(3-carbamoylphenyl)-2-(methylamino)propanoic acid, (2R)-2-amino-3-(3-carbamoylphenyl)propanoic acid, (2S)-2-amino-3-[3-(benzylcarbamoyl)phenyl]propanoic acid, (2S)-2-amino-3-[3-(dimethylcarbamoyl)phenyl]propanoic acid, (2S)-3-(3-aminophenyl)-2-(methylamino)propanoic acid, (2S)-3-(4-carbamoylphenyl)-2-(methylamino)propanoic acid, (2S)-2-amino-3-[4-(dimethylcarbamoyl)phenyl]propanoic acid, (2S)-2-amino-3-{4-[(2,5,8,11-tetraoxatridecan-13-yl)carbamoyl]phenyl}propanoic acid, (2S)-2-amino-4-(2-aminopyridin-3-yl)butanoic acid, (2S)-6-(carbamoylamino)-2-(methylamino)hexanoic acid, 4-[(3S)-3-amino-3-carboxypropyl]benzoic acid, (2S)-2-(methylamino)-6-[(1H-1,2,3,4-tetrazol-5-yl)amino]hexanoic acid, (2S)-6-(2-acetamidoacetamido)-2-(methylamino)hexanoic acid, (2S)-6-(3-carboxypropanamido)-2-(methylamino)hexanoic acid, (2S)-6-(cyclopropylformamido)-2-(methylamino)hexanoic acid, (2S)-2-(methylamino)-6-propanamidohexanoic acid, (2S)-6-(3-hydroxypropanamido)-2-(methylamino)hexanoic acid, (2S)-2-(methylamino)-6-(2-methylpropanamido)hexanoic acid, (2S)-6-(3-methoxypropanamido)-2-(methylamino)hexanoic acid, (2S)-6-[(2S)-2-hydroxypropanamido]-2-(methylamino)hexanoic acid, (2S)-6-[(2R)-2-hydroxypropanamido]-2-(methylamino)hexanoic acid, (2S)-2-(methylamino)-6-(phenylformamido)hexanoic acid, (2S)-6-(2-hydroxyacetamido)-2-(methylamino)hexanoic acid, (2S)-6-(2-methoxyacetamido)-2-(methylamino)hexanoic acid, (2S)-2-(methylamino)-6-(morpholin-4-yl)hexanoic acid, (2S)-6-methanesulfonamido-2-(methylamino)hexanoic acid, (2S)-2-(methylamino)-6-(2-oxoimidazolidin-1-yl)hexanoic acid, (2S)-2-(methylamino)-6-(2-oxopyrrolidin-1-yl)hexanoic acid, (2S)-2-(methylamino)-6-(2-oxo-1,3-diazinan-1-yl)hexanoic acid, (2S)-2-amino-3-{4′-cyano-[1,1′-biphenyl]-3-yl}propanoic acid, (2S)-2-(methylamino)-6-(1H-1,2,3-triazol-1-yl)hexanoic acid, (2S)-5-acetamido-2-(methylamino)pentanoic acid, (2S)-2-amino-5-methanesulfonamidopentanoic acid, (2S)-2-amino-4-{[(pyridin-4-yl)methyl]carbamoyl}butanoic acid, (2S)-2-amino-4-{[(1s,3s)-3-aminocyclobutyl]carbamoyl}butanoic acid, (2S)-4-[(carbamoylmethyl)carbamoyl]-2-(methylamino)butanoic acid, (2S)-2-amino-4-{[(pyrimidin-5-yl)methyl]carbamoyl}butanoic acid, (2S)-2-amino-4-{[(3S)-pyrrolidin-3-yl]carbamoyl}butanoic acid, (2S)-2-amino-3-(5,6,7,8-tetrahydronaphthalen-2-yl)propanoic acid, (2S)-2-amino-3-[5-(carboxymethoxy)-1H-indol-3-yl]propanoic acid, (2S)-2-amino-3-{3′-methanesulfonyl-[1,1′-biphenyl]-4-yl}propanoic acid, (2S)-2-amino-3-methanesulfonamidopropanoic acid, (2S)-3-(2-acetamidoacetamido)-2-(methylamino)propanoic acid, (2S)-2-amino-3-[3-(aminomethyl)phenyl]propanoic acid, (2S)-3-(4-aminophenyl)-2-(methylamino)propanoic acid, (S)-2-amino-3-(isoxazol-4-yl)propanoic acid, (2S)-3-hydroxy-2-(methylamino)propanoic acid, (S)-2-amino-3-(3-chlorophenyl)propanoic acid, (S)-2-amino-4-phenylbutanoic acid, (S)-2-amino-4-(4-fluorophenyl)butanoic acid, (S)-2-amino-4-(4-aminophenyl)butanoic acid, (S)-2-(methylamino)-3-(6-oxo-1,6-dihydropyridin-3-yl)propanoic acid, (S)-3-(4-methoxyphenyl)-2-(methylamino)propanoic acid, (S)-2-amino-3-(6-oxo-1,6-dihydropyridin-3-yl)propanoic acid, (S)-2-amino-3-(5,6-dimethoxypyridin-3-yl)propanoic acid, (S)-2-amino-3-(2-(methylcarbamoyl)pyridin-4-yl)propanoic acid, (S)-2-amino-3-(4-(methylsulfonyl)phenyl)propanoic acid, (S)-4-(2-carboxy-2-(methylamino)ethyl)benzoic acid, (S)-3-(4-acetamidophenyl)-2-aminopropanoic acid, (S)-2-amino-3-(6-methoxypyridin-3-yl)propanoic acid, (S)-3-(2-carboxy-2-(methylamino)ethyl)benzoic acid, (S)-5-(2-amino-2-carboxyethyl)nicotinic acid, (S)-3-(3-amino-3-carboxypropyl)benzoic acid, (S)-2-amino-3-(2-methoxypyridin-4-yl)propanoic acid, (S)-5-(2-amino-2-carboxyethyl)-2-hydroxybenzoic acid, (S)-2-amino-3-(4-hydroxy-3-methylphenyl)propanoic acid, (S)-3-(2-aminopyridin-4-yl)-2-(methylamino)propanoic acid, (S)-3-(4-(aminomethyl)phenyl)-2-(methylamino)propanoic acid, (S)-3-(3-((3-aminopropyl)carbamoyl)phenyl)-2-(methylamino)propanoic acid, (S)-3-(3-((2-aminoethyl)carbamoyl)phenyl)-2-(methylamino)propanoic acid, (S)-2-amino-3-(6-(methylamino)pyridin-3-yl)propanoic acid, (S)-3-(3-(aminomethyl)phenyl)-2-(methylamino)propanoic acid, (2S,4R)-4-hydroxypyrrolidine-2-carboxylic acid, (2R,4S)-4-aminopyrrolidine-2-carboxylic acid, (2R,4R)-4-hydroxypyrrolidine-2-carboxylic acid, (S)-2-(methylamino)pentanoic acid, (S)-2-amino-3-cyclobutylpropanoic acid, (S)-2-amino-3-cyclopentylpropanoic acid, (S)-2-amino-4-ethylhexanoic acid, (S)-2-amino-7,7,7-trifluoroheptanoic acid, (S)-2-amino-4-cyclobutylbutanoic acid, (S)-2-amino-4-cyclopentylbutanoic acid, (S)-2-amino-3-(1-methyl-1H-pyrazol-4-yl)propanoic acid, (S)-2-amino-3-(oxazol-5-yl)propanoic acid, (S)-2-amino-3-(oxazol-4-yl)propanoic acid, (S)-2-amino-3-(1H-pyrazol-4-yl)propanoic acid, (S)-2-amino-3-(5-hydroxy-1H-indol-3-yl)propanoic acid, (S)-2-amino-3-(naphthalen-1-yl)propanoic acid, (S)-2-(methylamino)-3-(naphthalen-2-yl)propanoic acid, (S)-2-amino-3-(7-methoxy-1H-indol-3-yl)propanoic acid, (S)-2-amino-3-(quinolin-6-yl)propanoic acid, (S)-2-amino-3-(1H-pyrrolo[3,2-c]pyridin-3-yl)propanoic acid, (S)-2-amino-3-(1-methyl-1H-pyrrolo[2,3-b]pyridin-3-yl)propanoic acid, (2S)-2-amino-3-[1-(carboxymethyl)-1H-indol-3-yl]propanoic acid, (S)-3-(1H-indazol-5-yl)-2-(methylamino)propanoic acid, (S)-2-amino-3-(5-(benzyloxy)-1H-indol-3-yl)propanoic acid, (2S,3S)-2-amino-3-hydroxybutanoic acid, (S)-2-amino-3-(tetrahydro-2H-pyran-4-yl)propanoic acid, (S)-2-amino-3-((1r,4S)-4-methoxycyclohexyl)propanoic acid, (S)-2-amino-3-((1r,4S)-4-hydroxycyclohexyl)propanoic acid, (2S)-2-amino-3-butoxypropanoic acid, (2S)-2-amino-3-(pentyloxy)propanoic acid, (2S)-2-amino-4-propoxybutanoic acid, (2S)-2-amino-4-ethoxybutanoic acid, (2S)-2-amino-4-butoxybutanoic acid, (2S)-2-amino-3-(cyclopentyloxy)propanoic acid, (2S)-2-amino-3-(cyclobutylmethoxy)propanoic acid, (2S)-2-amino-4-(cyclopentyloxy)butanoic acid, (2S)-2-amino-4-(propan-2-yloxy)butanoic acid, (S)-2-amino-3-(methyl(phenyl)amino)propanoic acid, (S)-4-acetamido-2-aminobutanoic acid, (R)-2-amino-3-(piperidin-4-yl)propanoic acid, (R)-2,4-diaminobutanoic acid, (R)-2,7-diaminoheptanoic acid, (S)-2,7-diaminoheptanoic acid, (S)-2-amino-3-(1-(methylsulfonyl)piperidin-4-yl)propanoic acid, (R)-2-amino-5-ureidopentanoic acid, (2S)-2-amino-6-(carbamoylamino)hexanoic acid, (S)-2-(methylamino)hexanedioic acid, (S)-6-(dimethylamino)-2-(methylamino)-6-oxohexanoic acid, (2R)-2-(methylamino)butanedioic acid, (S)-2-amino-3-(3-carbamoylphenyl)propanoic acid, (S)-2-amino-3-(4-carbamoylphenyl)propanoic acid, (R)-3-(4-carbamoylphenyl)-2-(methylamino)propanoic acid, (R)-3-(3-carbamoylphenyl)-2-(methylamino)propanoic acid, (R)-2-amino-3-(3-(aminomethyl)phenyl)propanoic acid, (S)-3-(4-(acetamidomethyl)phenyl)-2-(methylamino)propanoic acid, 2-{[2-(N-methylmethanesulfonamido)ethyl]amino}acetic acid, (S)-2-amino-4-ureidobutanoic acid, (S)-2-amino-5-(cyclohexanecarboxamido)pentanoic acid, (S)-2-amino-3-(3-((3-aminopropyl)carbamoyl)phenyl)propanoic acid, (S)-2-amino-3-(7-chloro-1-methyl-1H-indol-3-yl)propanoic acid, (S)-2-amino-3-(4-(1-methyl-1H-pyrazol-4-yl)phenyl)propanoic acid, (S)-2-amino-3-(4-(pyridin-3-yl)phenyl)propanoic acid, (S)-3-(isoquinolin-7-yl)-2-(methylamino)propanoic acid, (S)-2-amino-3-(2-phenylpyridin-4-yl)propanoic acid, (S)-2-amino-3-(quinolin-5-yl)propanoic acid, (S)-2-amino-3-(3-((2-aminoethyl)carbamoyl)phenyl)propanoic acid, 2-[(2-carbamoylethyl)amino]acetic acid, (S)-2-amino-3-(4-(pyridin-2-yl)phenyl)propanoic acid, (2S)-2-amino-4-[(2-carbamoylethyl)carbamoyl]butanoic acid, (S)-3-(4-(1-acetylpiperidin-4-yl)phenyl)-2-aminopropanoic acid, (S)-2-(methylamino)-3-(quinolin-7-yl)propanoic acid, (S)-2-amino-4-oxo-4-(pyrrolidin-1-yl)butanoic acid, (2S)-2-amino-3-[1-(3-aminopropyl)-1H-indol-3-yl]propanoic acid, 2-[(2-{[1,1′-biphenyl]-4-yl}ethyl)amino]acetic acid, (S)-2-amino-3-(5,6-dichloro-1H-indol-3-yl)propanoic acid, (S)-2-amino-3-(4-(piperidin-4-yl)phenyl)propanoic acid, (S)-2-amino-3-(6-carbamoylpyridin-3-yl)propanoic acid, (S)-2-amino-3-(1H-pyrrolo[3,2-b]pyridin-3-yl)propanoic acid, (R)-3-(2-amino-2-carboxyethyl)benzoic acid, (R)-4-(2-amino-2-carboxyethyl)benzoic acid, (S)-2-amino-3-(5-methyl-1H-imidazol-4-yl)propanoic acid, (2R)-2-amino-3-sulfamoylpropanoic acid, (S)-2-amino-3-(5-phenylpyridin-3-yl)propanoic acid, (S)-3-(3,4-dichlorophenyl)-2-(methylamino)propanoic acid, (S)-2-amino-3-(4-(2-amino-2-oxoethyl)phenyl)propanoic acid, (S)-2-amino-3-(2-carbamoylpyridin-3-yl)propanoic acid, (S)-2-amino-3-(5,6-dimethylpyridin-3-yl)propanoic acid, (S)-2-amino-3-(2,6-dimethylpyridin-4-yl)propanoic acid, (R)-2-amino-3-(2-aminopyridin-4-yl)propanoic acid, (S)-2-amino-4-(5-(trifluoromethyl)pyridin-3-yl)butanoic acid, (S)-2-amino-4-(2-(trifluoromethyl)pyridin-4-yl)butanoic acid, (R)-3-cyclobutyl-2-(methylamino)propanoic acid, (S)-3-(2,3-dichlorophenyl)-2-(methylamino)propanoic acid, (S)-2-amino-3-(1,4,5,6-tetrahydrocyclopenta[c]pyrazol-3-yl)propanoic acid, (S)-2-amino-3-(4-(tetrahydro-2H-pyran-4-yl)phenyl)propanoic acid, (S)-2-amino-4-(3-(pentafluoro-16-sulfaneyl)phenyl)butanoic acid, (2S,4R)-4-(2-chlorophenoxy)pyrrolidine-2-carboxylic acid, (2S,4R)-4-(2-fluorophenoxy)pyrrolidine-2-carboxylic acid, (2S,4R)-4-(3-chlorophenoxy)pyrrolidine-2-carboxylic acid, (2S,4R)-4-(3-fluorophenoxy)pyrrolidine-2-carboxylic acid, (2S,4R)-4-(4-chlorophenoxy)pyrrolidine-2-carboxylic acid, (2S,4R)-4-(4-fluorophenoxy)pyrrolidine-2-carboxylic acid, (S)-2-amino-3-(2-fluoro-[1,1′-biphenyl]-4-yl)propanoic acid, (S)-2-amino-3-(6-(2-aminoethoxy)pyridin-3-yl)propanoic acid, (S)-2-amino-4-(dimethylamino)butanoic acid, (S)-2-amino-3-(4-(quinolin-4-yl)phenyl)propanoic acid, (S,E)-2-amino-3-(4-(2-methylguanidino)phenyl)propanoic acid, (S)-2-amino-3-(4-((4,5-dihydro-1H-imidazol-2-yl)amino)phenyl)propanoic acid, (S)-2-amino-3-(2-(dimethylcarbamoyl)-1H-imidazol-4-yl)propanoic acid, (S)-2-amino-3-(2-(methylcarbamoyl)-1H-imidazol-4-yl)propanoic acid, (2S)-2-amino-3-{[(azetidin-3-yl)methyl]carbamoyl}propanoic acid, (S)-2-amino-3-(isoquinolin-8-yl)propanoic acid, (2S,4R)-4-(pyridin-2-yloxy)pyrrolidine-2-carboxylic acid, (2S,4R)-4-(pyridin-3-yloxy)pyrrolidine-2-carboxylic acid, (S)-2-(methylamino)-3-(thiazol-2-yl)propanoic acid, (S)-3-(5-chloropyridin-3-yl)-2-(methylamino)propanoic acid, (S)-2-(methylamino)-3-(5-(trifluoromethyl)pyridin-3-yl)propanoic acid, (S)-3-(2,6-dichloropyridin-4-yl)-2-(methylamino)propanoic acid, (S)-2-(methylamino)-3-(tetrahydro-2H-pyran-4-yl)propanoic acid, (R)-2-(methylamino)-3-(piperidin-4-yl)propanoic acid, (R)-2-(methylamino)-3-(tetrahydro-2H-pyran-4-yl)propanoic acid, (2R)-6-amino-2-(methylamino)hexanoic acid, (S)-3-(2,3-dimethylphenyl)-2-(methylamino)propanoic acid, (S)-3-(3,5-bis(trifluoromethyl)phenyl)-2-(methylamino)propanoic acid, (S)-3-(3-chloro-5-(trifluoromethyl)phenyl)-2-(methylamino)propanoic acid, (S)-3-(3-ethylphenyl)-2-(methylamino)propanoic acid, (S)-6,6,6-trifluoro-2-(methylamino)hexanoic acid, (S)-2,5-bis(methylamino)pentanoic acid, (2S)-2-amino-4-(cyclohexyloxy)butanoic acid, (2S)-2-amino-4-(oxan-4-yloxy)butanoic acid, (2S)-2-amino-3-(2,2-dimethylpropoxy)propanoic acid, (2S)-3-(carboxymethoxy)-2-(methylamino)propanoic acid, (R)-3-(3,4-dichlorophenyl)-2-(methylamino)propanoic acid, (R)-2-amino-3-(6-oxo-1,6-dihydropyridin-3-yl)propanoic acid, (R)-2-amino-4-(4-aminophenyl)butanoic acid, (R)-3-(6-methoxypyridin-3-yl)-2-(methylamino)propanoic acid, (S)-3-(2-methoxypyridin-4-yl)-2-(methylamino)propanoic acid, (R)-2-(methylamino)-3-(6-oxo-1,6-dihydropyridin-3-yl)propanoic acid, (S)-5-(2-carboxy-2-(methylamino)ethyl)-2-hydroxybenzoic acid, (S)-4-(4-fluorophenyl)-2-(methylamino)butanoic acid, (S)-2-(methylamino)-3-(oxazol-4-yl)propanoic acid, (S)-3-(1-methyl-1H-pyrazol-4-yl)-2-(methylamino)propanoic acid, (R)-2-amino-3-(5-phenylpyridin-3-yl)propanoic acid, (R)-2-amino-3-(2-phenylpyridin-4-yl)propanoic acid, (S)-2-(methylamino)-3-(quinolin-6-yl)propanoic acid, (R)-2-amino-3-(quinolin-5-yl)propanoic acid, (S)-2-amino-3-(isoquinolin-7-yl)propanoic acid, (S)-3-(isoquinolin-8-yl)-2-(methylamino)propanoic acid, (2R,4R)-4-aminopyrrolidine-2-carboxylic acid, (2S)-4-ethoxy-2-(methylamino)butanoic acid, (2S)-3-butoxy-2-(methylamino)propanoic acid, (2S)-2-amino-3-sulfamoylpropanoic acid, (2S)-2-(methylamino)-4-propoxybutanoic acid, (2S)-2-amino-3-[4-(aminomethyl)phenyl]propanoic acid and (2S)-2-amino-7-{[4-(carboxymethyl)piperazine-1-carbonyl]amino}heptanoic acid.

In one aspect, the novel amino acid derivative of this disclosure may contain any functional group such as carboxyl group and amino groups and any functional group in their side chains protected by conventional or any known protecting groups (e.g. Fmoc or Boc).

In other words, the novel amino acid derivative which side chains protected by conventional or any known protecting groups of this disclosure is at least one selected from the group consisting of (2S)-2-[[(9H-fluoren-9-ylmethoxy)carbonyl]amino]-6-(4-methylpiperazine-1-carbonylamino)hexanoic acid hydrochloride, (2S)-6-({4-[2-(tert-butoxy)-2-oxoethyl]piperazine-1-carbonyl}amino)-2-({[(9H-fluoren-9-yl)methoxy]carbonyl}amino)hexanoic acid, (2S)-2-({[(9H-fluoren-9-yl)methoxy]carbonyl}amino)-6-({methyl[(2S,3R,4R,5R)-2,3,4,5-tetrahydroxy-6-{[tris(propan-2-yl)silyl]oxy}hexyl]carbamoyl}amino)hexanoic acid”, (2R)-6-({[(3R)-1-[2-(tert-butoxy)-2-oxoethyl]pyrrolidin-3-yl]carbamoyl}amino)-2-({[(9H-fluoren-9-yl)methoxy]carbonyl}amino)hexanoic acid, (2S)-6-[(4-{2-[(tert-butyldimethylsilyl)oxy]ethyl}piperazine-1-carbonyl)amino]-2-({[(9H-fluoren-9-yl)methoxy]carbonyl}amino)hexanoic acid, (2R)-6-({4-[2-(tert-butoxy)-2-oxoethyl]piperazine-1-carbonyl}amino)-2-({[(9H-fluoren-9-yl)methoxy]carbonyl}amino)hexanoic acid, (2S)-6-({4-[2-(tert-butoxy)-2-oxoethyl]piperazine-1-carbonyl}amino)-2-({[(9H-fluoren-9-yl)methoxy]carbonyl}(methyl)amino)hexanoic acid, (2R)-6-({4-[2-(tert-butoxy)-2-oxoethyl]piperazine-1-carbonyl}amino)-2-({[(9H-fluoren-9-yl)methoxy]carbonyl}(methyl)amino)hexanoic acid, (2S)-2-({[(9H-fluoren-9-yl)methoxy]carbonyl}(methyl)amino)-6-({methyl[(2S,3R,4R,5R)-2,3,4,5-tetrahydroxy-6-{[tris(propan-2-yl)silyl]oxy}hexyl]carbamoyl}amino)hexanoic acid, (2R)-2-({[(9H-fluoren-9-yl)methoxy]carbonyl}amino)-6-({methyl[(2S,3R,4R,5R)-2,3,4,5-tetrahydroxy-6-{[tris(propan-2-yl)silyl]oxy}hexyl]carbamoyl}amino)hexanoic acid, (2R)-6-[(4-{2-[(tert-butyldimethylsilyl)oxy]ethyl}piperazine-1-carbonyl)amino]-2-({[(9H-fluoren-9-yl)methoxy]carbonyl}amino)hexanoic acid, (2S)-3-({4-[2-(tert-butoxy)-2-oxoethyl]piperazine-1-carbonyl}amino)-2-({[(9H-fluoren-9-yl)methoxy]carbonyl}amino)propanoic acid, (2S)-3-({4-[2-(tert-butoxy)-2-oxoethyl]piperazine-1-carbonyl}amino)-2-({[(9H-fluoren-9-yl)methoxy]carbonyl}(methyl)amino)propanoic acid, (2S,4R)-4-({4-[2-(tert-butoxy)-2-oxoethyl]piperazine-1-carbonyl}amino)-1-{[(9H-fluoren-9-yl)methoxy]carbonyl}pyrrolidine-2-carboxylic acid, (2S,4S)-4-({4-[2-(tert-butoxy)-2-oxoethyl]piperazine-1-carbonyl}amino)-1-{[(9H-fluoren-9-yl)methoxy]carbonyl}pyrrolidine-2-carboxylic acid, (2S)-2-({[(9H-fluoren-9-yl)methoxy]carbonyl}amino)-6-[(4-{2-oxo-2-[(2-phenylpropan-2-yl)oxy]ethyl}piperazine-1-carbonyl)amino]hexanoic acid, (2S)-7-({4-[2-(tert-butoxy)-2-oxoethyl]piperazine-1-carbonyl}amino)-2-({[(9H-fluoren-9-yl)methoxy]carbonyl}amino)heptanoic acid, (2S)-3-{4-[2-({4-[2-(tert-butoxy)-2-oxoethyl]piperazine-1-carbonyl}amino)ethoxy]phenyl}-2-({[(9H-fluoren-9-yl)methoxy]carbonyl}amino)propanoic acid, (2R)-6-[({1-[2-(tert-butoxy)-2-oxoethyl]piperidin-4-yl}carbamoyl)amino]-2-({[(9H-fluoren-9-yl)methoxy]carbonyl}amino)hexanoic acid, (2R)-6-{[({1-[2-(tert-butoxy)-2-oxoethyl]piperidin-4-yl}oxy)carbonyl]amino}-2-({[(9H-fluoren-9-yl)methoxy]carbonyl}amino)hexanoic acid, (2S)-6-({1-[2-(tert-butoxy)-2-oxoethyl]piperidin-4-yl}formamido)-2-({[(9H-fluoren-9-yl)methoxy]carbonyl}amino)hexanoic acid, (2R)-6-{1-[2-(tert-butoxy)-2-oxoethyl]piperidine-4-sulfonamido}-2-({[(9H-fluoren-9-yl)methoxy]carbonyl}amino)hexanoic acid, (2S)-3-[4-(2-{4-[2-(tert-butoxy)-2-oxoethyl]piperazin-1-yl}-2-oxoethoxy)phenyl]-2-({[(9H-fluoren-9-yl)methoxy]carbonyl}amino)propanoic acid, (2R)-6-({1-[2-(tert-butoxy)-2-oxoethyl]piperidin-4-yl}formamido)-2-({[(9H-fluoren-9-yl)methoxy]carbonyl}amino)hexanoic acid, (2R)-6-(2-{1-[2-(tert-butoxy)-2-oxoethyl]piperidin-4-yl}acetamido)-2-({[(9H-fluoren-9-yl)methoxy]carbonyl}amino)hexanoic acid, (2S)-6-{1-[2-(tert-butoxy)-2-oxoethyl]piperidine-4-sulfonamido}-2-({[(9H-fluoren-9-yl)methoxy]carbonyl}amino)hexanoic acid, (2S)-3-({1-[2-(tert-butoxy)-2-oxoethyl]piperidin-4-yl}formamido)-2-({[(9H-fluoren-9-yl)methoxy]carbonyl}amino)propanoic acid, (2R)-6-{4-[2-(tert-butoxy)-2-oxoethyl]piperazin-1-yl}-2-({[(9H-fluoren-9-yl)methoxy]carbonyl}amino)-6-oxohexanoic acid, (2R)-5-({1-[2-(tert-butoxy)-2-oxoethyl]piperidin-4-yl}carbamoyl)-2-({[(9H-fluoren-9-yl)methoxy]carbonyl}amino)pentanoic acid, (2R)-6-{[({1,3-bis[(2,2-dimethyl-1,3-dioxan-5-yl)methoxy]propan-2-yl}oxy)carbonyl]amino}-2-({[(9H-fluoren-9-yl)methoxy]carbonyl}amino)hexanoic acid, (2R)-2-({[(9H-fluoren-9-yl)methoxy]carbonyl}amino)-6-{4-[(2-methoxyethoxy)methyl]-2-oxo-3,6,9-trioxa-1-azadecan-1-yl}hexanoic acid, (2S)-6-{[({1,3-bis[(2,2-dimethyl-1,3-dioxan-5-yl)methoxy]propan-2-yl}oxy)carbonyl]amino}-2-({[(9H-fluoren-9-yl)methoxy]carbonyl}amino)hexanoic acid, (2S)-6-({5-[(tert-butoxy)carbonyl]pyridin-2-yl}amino)-2-({[(9H-fluoren-9-yl)methoxy]carbonyl}amino)hexanoic acid, (2S)-2-({[(9H-fluoren-9-yl)methoxy]carbonyl}(methyl)amino)-6-{[(prop-2-en-1-yloxy)carbonyl]amino}hexanoic acid, “(2S)-6-acetamido-2-({[(9H-fluoren-9-yl)methoxy]carbonyl}(methyl)amino)hexanoic acid, (2S)-2-({[(9H-fluoren-9-yl)methoxy]carbonyl}(methyl)amino)-4-{[(prop-2-en-1-yloxy)carbonyl]amino}butanoic acid, (2S)-3-acetamido-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-{[(prop-2-en-1-yloxy)carbonyl]amino}propanoic acid, (2S)-3-{1-[2-(tert-butoxy)-2-oxoethyl]piperidin-4-yl}-2-({[(9H-fluoren-9-yl)methoxy]carbonyl}amino)propanoic acid, (2S)-2-{1-[2-(tert-butoxy)-2-oxoethyl]piperidin-4-yl}-2-({[(9H-fluoren-9-yl)methoxy]carbonyl}amino)acetic acid, (2R)-3-{1-[2-(tert-butoxy)-2-oxoethyl]piperidin-4-yl}-2-({[(9H-fluoren-9-yl)methoxy]carbonyl}amino)propanoic acid, (2S)-3-{4-[({4-[2-(tert-butoxy)-2-oxoethyl]piperazine-1-carbonyl}amino)methyl]phenyl}-2-({[(9H-fluoren-9-yl)methoxy]carbonyl}amino)propanoic acid, (2R)-3-{4-[({4-[2-(tert-butoxy)-2-oxoethyl]piperazine-1-carbonyl}amino)methyl]phenyl}-2-({[(9H-fluoren-9-yl)methoxy]carbonyl}amino)propanoic acid, (2S)-3-{3-[({4-[2-(tert-butoxy)-2-oxoethyl]piperazine-1-carbonyl}amino)methyl]phenyl}-2-({[(9H-fluoren-9-yl)methoxy]carbonyl}amino)propanoic acid, (2S)-3-(6-{[2-(tert-butoxy)-2-oxoethyl]amino}pyridin-3-yl)-2-({[(9H-fluoren-9-yl)methoxy]carbonyl}amino)propanoic acid, (2R)-3-{3-[(tert-butoxy)carbonyl]phenyl}-2-({[(9H-fluoren-9-yl)methoxy]carbonyl}amino)propanoic acid, (2S)-3-{1-[(tert-butoxy)carbonyl]-1H-pyrrolo[3,2-c]pyridin-3-yl}-2-({[(9H-fluoren-9-yl)methoxy]carbonyl}amino)propanoic acid, (2S)-2-({[(9H-fluoren-9-yl)methoxy]carbonyl}amino)-3-[1-(triphenylmethyl)-1H-pyrazol-3-yl]propanoic acid, (2S)-3-(3,4-dimethylphenyl)-2-({[(9H-fluoren-9-yl)methoxy]carbonyl}(methyl)amino)propanoic acid, (2S)-3-(3,4-dimethoxyphenyl)-2-({[(9H-fluoren-9-yl)methoxy]carbonyl}(methyl)amino)propanoic acid, (2S)-3-{1-[(tert-butoxy)carbonyl]-1H-indol-6-yl}-2-({[(9H-fluoren-9-yl)methoxy]carbonyl}amino)propanoic acid, (2S)-3-{1-[(tert-butoxy)carbonyl]-5-methyl-1H-imidazol-4-yl}-2-({[(9H-fluoren-9-yl)methoxy]carbonyl}amino)propanoic acid, (2S)-2-({[(9H-fluoren-9-yl)methoxy]carbonyl}amino)-3-(2-oxo-1,2-dihydropyridin-4-yl)propanoic acid, (2R)-3-(6-{[(tert-butoxy)carbonyl]amino}pyridin-3-yl)-2-({[(9H-fluoren-9-yl)methoxy]carbonyl}amino)propanoic acid, (2S)-2-({[(9H-fluoren-9-yl)methoxy]carbonyl}amino)-3-(6-hydroxy-1H-indol-3-yl)propanoic acid, (2S)-3-(2H-1,3-benzodioxol-5-yl)-2-({[(9H-fluoren-9-yl)methoxy]carbonyl}(methyl)amino)propanoic acid, (2S)-3-(4-ethylphenyl)-2-({[(9H-fluoren-9-yl)methoxy]carbonyl}(methyl)amino)propanoic acid, (2S)-2-({[(9H-fluoren-9-yl)methoxy]carbonyl}(methyl)amino)-4-(pyridin-3-yl)butanoic acid, (2S)-2-({[(9H-fluoren-9-yl)methoxy]carbonyl}(methyl)amino)-4-(pyridin-2-yl)butanoic acid, (2S)-2-({[(9H-fluoren-9-yl)methoxy]carbonyl}(methyl)amino)-4-(5-methoxypyridin-2-yl)butanoic acid, (2S)-2-({[(9H-fluoren-9-yl)methoxy]carbonyl}(methyl)amino)-4-(6-methoxypyridin-3-yl)butanoic acid, (2S)-2-({[(9H-fluoren-9-yl)methoxy]carbonyl}amino)-3-(1H-1,2,3-triazol-4-yl)propanoic acid, (2S)-2-({[(9H-fluoren-9-yl)methoxy]carbonyl}amino)-3-{4-[(prop-2-en-1-yloxy)carbonyl]phenyl}propanoic acid, (2S)-3-{6-[(tert-butoxy)carbonyl]pyridin-3-yl}-2-({[(9H-fluoren-9-yl)methoxy]carbonyl}amino)propanoic acid, (2S)-6-({bis[2-(tert-butoxy)-2-oxoethyl]carbamoyl}amino)-2-({[(9H-fluoren-9-yl)methoxy]carbonyl}amino)hexanoic acid, (2S)-2-({[(9H-fluoren-9-yl)methoxy]carbonyl}amino)-6-({4-[2-oxo-2-(prop-2-en-1-yloxy)ethyl]piperazine-1-carbonyl}amino)hexanoic acid, (2S)-2-({[(9H-fluoren-9-yl)methoxy]carbonyl}amino)-6[(4-{2-[(3-methylpentan-3-yl)oxy]-2-oxoethyl}piperazine-1-carbonyl)amino]hexanoic acid, (2S)-5-{4-[2-(tert-butoxy)-2-oxoethyl]piperazin-1-yl}-2-({[(9H-fluoren-9-yl)methoxy]carbonyl}amino)-5-oxopentanoic acid, (2R)-3-({1-[2-(tert-butoxy)-2-oxoethyl]piperidin-4-yl}formamido)-2-({[(9H-fluoren-9-yl)methoxy]carbonyl}amino)propanoic acid, (2S)-3-({1-[2-(tert-butoxy)-2-oxoethyl]piperidin-4-yl}formamido)-2-({[(9H-fluoren-9-yl)methoxy]carbonyl}(methyl)amino)propanoic acid, (2S)-6-{4-[2-(tert-butoxy)-2-oxoethyl]piperazin-1-yl}-2-({[(9H-fluoren-9-yl)methoxy]carbonyl}amino)-6-oxohexanoic acid, (2S)-4-{bis[2-(tert-butoxy)-2-oxoethyl]carbamoyl}-2-({[(9H-fluoren-9-yl)methoxy]carbonyl}amino)butanoic acid, (2S)-2-({[(9H-fluoren-9-yl)methoxy]carbonyl}amino)-3-{1-[2-oxo-2-(prop-2-en-1-yloxy)ethyl]piperidin-4-yl}propanoic acid, 1-[2-(tert-butoxy)-2-oxoethyl]-4-({[(9H-fluoren-9-yl)methoxy]carbonyl}amino)piperidine-4-carboxylic acid, (2S)-3-{1-[2-(tert-butoxy)-2-oxoethyl]piperidin-4-yl}-2-({[(9H-fluoren-9-yl)methoxy]carbonyl}(methyl)amino)propanoic acid, (2S)-3-{4-[({4-[2-(tert-butoxy)-2-oxoethyl]piperazine-1-carbonyl}amino)methyl]phenyl}-2-({[(9H-fluoren-9-yl)methoxy]carbonyl}(methyl)amino)propanoic acid, (2R)-3-{4-[({4-[2-(tert-butoxy)-2-oxoethyl]piperazine-1-carbonyl}amino)methyl]phenyl}-2-({[(9H-fluoren-9-yl)methoxy]carbonyl}(methyl)amino)propanoic acid, (2S)-3-[3-(tert-butoxy)phenyl]-2-({[(9H-fluoren-9-yl)methoxy]carbonyl}(methyl)amino)propanoic acid, (2S)-3-{4-[2-({1-[2-(tert-butoxy)-2-oxoethyl]piperidin-4-yl}formamido)ethoxy]phenyl}-2-({[(9H-fluoren-9-yl)methoxy]carbonyl}amino)propanoic acid, (2S)-3-[4-(2-{[2-(tert-butoxy)-2-oxoethyl][(tert-butoxy)carbonyl]amino}ethoxy)phenyl]-2-({[(9H-fluoren-9-yl)methoxy]carbonyl}amino)propanoic acid, (2R)-6-[({1-[2-(tert-butoxy)-2-oxoethyl]azetidin-3-yl}carbamoyl)amino]-2-({[(9H-fluoren-9-yl)methoxy]carbonyl}amino)hexanoic acid, (2R)-6-[({1-[2-(tert-butoxy)-2-oxoethyl]piperidin-4-yl}carbamoyl)oxy]-2-({[(9H-fluoren-9-yl)methoxy]carbonyl}amino)hexanoic acid, (S)-2-((((9H-fluoren-9-yl)methoxy)carbonyl)amino)-3-((2-(tert-butoxy)-2-oxoethyl)(methyl)amino)propanoic acid, (2S)-2-({[(9H-fluoren-9-yl)methoxy]carbonyl}amino)-3-{methyl[2-oxo-2-(prop-2-en-1-yloxy)ethyl]amino}propanoic acid, (2S)-4-({4-[2-(tert-butoxy)-2-oxoethyl]piperazine-1-carbonyl}amino)-2-({[(9H-fluoren-9-yl)methoxy]carbonyl}amino)butanoic acid, (2S)-2-({[(9H-fluoren-9-yl)methoxy]carbonyl}(methyl)amino)-6-(N-methylacetamido)hexanoic acid, 2-({[(9H-fluoren-9-yl)methoxy]carbonyl}[4-(N-methylmethanesulfonamido)butyl]amino)acetic acid, 2-({[(9H-fluoren-9-yl)methoxy]carbonyl}(4-methanesulfonamidobutyl)amino)acetic acid, 2-({[(9H-fluoren-9-yl)methoxy]carbonyl}(3-methanesulfonamidopropyl)amino)acetic acid, 2-({[(9H-fluoren-9-yl)methoxy]carbonyl}[3-(N-methylmethanesulfonamido)propyl]amino)acetic acid, 2-[(4-acetamidobutyl)({[(9H-fluoren-9-yl)methoxy]carbonyl})amino]acetic acid, 2-({[(9H-fluoren-9-yl)methoxy]carbonyl}[3-(N-methylacetamido)propyl]amino)acetic acid, 2-[(3-{[(tert-butoxy)carbonyl](methyl)amino}propyl)({[(9H-fluoren-9-yl)methoxy]carbonyl})amino]acetic acid, (2S)-3-{1-[(tert-butoxy)carbonyl]-5-phenoxy-1H-indol-3-yl}-2-({[(9H-fluoren-9-yl)methoxy]carbonyl}amino)propanoic acid, (2S)-2-({[(9H-fluoren-9-yl)methoxy]carbonyl}amino)-3-[6-(pyridin-3-yl)-1H-indol-3-yl]propanoic acid, (2S)-2-({[(9H-fluoren-9-yl)methoxy]carbonyl}amino)-3-[6-(pyridin-4-yl)-1H-indol-3-yl]propanoic acid, (2S)-2-({[(9H-fluoren-9-yl)methoxy]carbonyl}amino)-3-[6-(pyrimidin-5-yl)-1H-indol-3-yl]propanoic acid, (2S)-3-{1-[(tert-butoxy)carbonyl]-6-(1-methyl-1H-pyrazol-4-yl)-1H-indol-3-yl}-2-({[(9H-fluoren-9-yl)methoxy]carbonyl}amino)propanoic acid, (2S)-3-(3-carbamoyl-4-hydroxyphenyl)-2-({[(9H-fluoren-9-yl)methoxy]carbonyl}amino)propanoic acid, (2S)-3-[3-(dimethylcarbamoyl)-4-hydroxyphenyl]-2-({[(9H-fluoren-9-yl)methoxy]carbonyl}amino)propanoic acid, (2S)-2-({[(9H-fluoren-9-yl)methoxy]carbonyl}amino)-3-[4-(2-{[(prop-2-en-1-yloxy)carbonyl]amino}ethoxy)phenyl]propanoic acid, (2S)-3-[4-(2-acetamidoethoxy)phenyl]-2-({[(9H-fluoren-9-yl)methoxy]carbonyl}amino)propanoic acid, (2S)-3-(3-carbamoylpyridin-2-yl)-2-({[(9H-fluoren-9-yl)methoxy]carbonyl}amino)propanoic acid, (2S)-2-({[(9H-fluoren-9-yl)methoxy]carbonyl}amino)-3-(6-phenylpyridin-3-yl)propanoic acid, (2R)-2-({[(9H-fluoren-9-yl)methoxy]carbonyl}amino)-3-(6-phenylpyridin-3-yl)propanoic acid, (2S)-3-(2-carbamoylpyridin-4-yl)-2-({[(9H-fluoren-9-yl)methoxy]carbonyl}amino)propanoic acid, (2S)-3-(3-carbamoylpyridin-4-yl)-2-({[(9H-fluoren-9-yl)methoxy]carbonyl}amino)propanoic acid, (2S)-2-({[(9H-fluoren-9-yl)methoxy]carbonyl}amino)-3-(1H-indazol-5-yl)propanoic acid, (2R)-2-({[(9H-fluoren-9-yl)methoxy]carbonyl}amino)-3-(1H-indazol-5-yl)propanoic acid, (2S)-3-(1-acetylpiperidin-4-yl)-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-{1-[(prop-2-en-1-yloxy)carbonyl]piperidin-4-yl}propanoic acid, (2S)-3-{1-[(tert-butoxy)carbonyl]piperidin-4-yl}-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-{1-[1-(triphenylmethyl)-1H-pyrazole-4-carbonyl]piperidin-4-yl}propanoic acid, (2S)-2-({[(9H-fluoren-9-yl)methoxy]carbonyl}(methyl)amino)-3-{1-[3-(triphenylmethoxy)propanoyl]piperidin-4-yl}propanoic acid, (2S)-3-{1-[2-(tert-butoxy)acetyl]piperidin-4-yl}-2-({[(9H-fluoren-9-yl)methoxy]carbonyl}(methyl)amino)propanoic acid, (2S)-2-({[(9H-fluoren-9-yl)methoxy]carbonyl}amino)-7-methoxyheptanoic acid, (2S)-3-[3′-(tert-butoxy)-[1,1′-biphenyl]-4-yl]-2-({[(9H-fluoren-9-yl)methoxy]carbonyl}amino)propanoic acid, (2S)-2-({[(9H-fluoren-9-yl)methoxy]carbonyl}amino)-4-methanesulfonamidobutanoic acid, (2R)-2-({[(9H-fluoren-9-yl)methoxy]carbonyl}(methyl)amino)-3-{[(prop-2-en-1-yloxy)carbonyl]amino}propanoic acid, (2S)-3-{[(tert-butoxy)carbonyl]amino}-2-({[(9H-fluoren-9-yl)methoxy]carbonyl}(methyl)amino)propanoic acid, (2R)-2-({[(9H-fluoren-9-yl)methoxy]carbonyl}amino)-3-{3-[2-oxo-2-(prop-2-en-1-yloxy)ethyl]phenyl}propanoic acid, (2S)-2-({[(9H-fluoren-9-yl)methoxy]carbonyl}amino)-3-{3-[2-oxo-2-(prop-2-en-1-yloxy)ethyl]phenyl}propanoic acid, (2R)-3-{3-[2-(tert-butoxy)-2-oxoethyl]phenyl}-2-({[(9H-fluoren-9-yl)methoxy]carbonyl}amino)propanoic acid, (2S)-3-{3-[2-(tert-butoxy)-2-oxoethyl]phenyl}-2-({[(9H-fluoren-9-yl)methoxy]carbonyl}amino)propanoic acid, (2R)-2-({[(9H-fluoren-9-yl)methoxy]carbonyl}amino)-3-{3-[2-oxo-2-(prop-2-en-1-yloxy)ethoxy]phenyl}propanoic acid, (2S)-2-({[(9H-fluoren-9-yl)methoxy]carbonyl}amino)-3-{3-[2-oxo-2-(prop-2-en-1-yloxy)ethoxy]phenyl}propanoic acid, (2R)-3-{3-[2-(tert-butoxy)-2-oxoethoxy]phenyl}-2-({[(9H-fluoren-9-yl)methoxy]carbonyl}amino)propanoic acid, (2S)-3-{3-[2-(tert-butoxy)-2-oxoethoxy]phenyl}-2-({[(9H-fluoren-9-yl)methoxy]carbonyl}amino)propanoic acid, (2S)-3-[3-(acetamidomethyl)phenyl]-2-({[(9H-fluoren-9-yl)methoxy]carbonyl}(methyl)amino)propanoic acid, (2S)-3-(3-carbamoylphenyl)-2-({[(9H-fluoren-9-yl)methoxy]carbonyl}(methyl)amino)propanoic acid, (2R)-3-(3-carbamoylphenyl)-2-({[(9H-fluoren-9-yl)methoxy]carbonyl}amino)propanoic acid, (2S)-3-[3-(benzylcarbamoyl)phenyl]-2-({[(9H-fluoren-9-yl)methoxy]carbonyl}amino)propanoic acid, (2S)-3-[3-(dimethylcarbamoyl)phenyl]-2-({[(9H-fluoren-9-yl)methoxy]carbonyl}amino)propanoic acid, (2S)-2-({[(9H-fluoren-9-yl)methoxy]carbonyl}(methyl)amino)-3-(3-{[(prop-2-en-1-yloxy)carbonyl]amino}phenyl)propanoic acid, (2S)-3-(4-carbamoylphenyl)-2-({[(9H-fluoren-9-yl)methoxy]carbonyl}(methyl)amino)propanoic acid, (2S)-3-[4-(dimethylcarbamoyl)phenyl]-2-({[(9H-fluoren-9-yl)methoxy]carbonyl}amino)propanoic acid, (2S)-2-({[(9H-fluoren-9-yl)methoxy]carbonyl}amino)-3-{4-[(2,5,8,11-tetraoxatridecan-13-yl)carbamoyl]phenyl}propanoic acid, (2S)-4-(2-{[(tert-butoxy)carbonyl]amino}pyridin-3-yl)-2-({[(9H-fluoren-9-yl)methoxy]carbonyl}amino)butanoic acid, (2S)-6-(carbamoylamino)-2-({[(9H-fluoren-9-yl)methoxy]carbonyl}(methyl)amino)hexanoic acid, (2S)-4-{4-[(tert-butoxy)carbonyl]phenyl}-2-({[(9H-fluoren-9-yl)methoxy]carbonyl}amino)butanoic acid, (2S)-2-({[(9H-fluoren-9-yl)methoxy]carbonyl}(methyl)amino)-6-[(2H-1,2,3,4-tetrazol-5-yl)amino]hexanoic acid, (2S)-6-{[(tert-butoxy)carbonyl](1H-1,2,3,4-tetrazol-5-yl)amino}-2-({[(9H-fluoren-9-yl)methoxy]carbonyl}(methyl)amino)hexanoic acid, (2S)-6-(2-acetamidoacetamido)-2-({[(9H-fluoren-9-yl)methoxy]carbonyl}(methyl)amino)hexanoic acid, (2S)-6-[4-(tert-butoxy)-4-oxobutanamido]-2-({[(9H-fluoren-9-yl)methoxy]carbonyl}(methyl)amino)hexanoic acid, (2S)-6-(cyclopropylformamido)-2-({[(9H-fluoren-9-yl)methoxy]carbonyl}(methyl)amino)hexanoic acid, (2S)-2-({[(9H-fluoren-9-yl)methoxy]carbonyl}(methyl)amino)-6-propanamidohexanoic acid, (2S)-2-({[(9H-fluoren-9-yl)methoxy]carbonyl}(methyl)amino)-6-[3-(triphenylmethoxy)propanamido]hexanoic acid, (2S)-2-({[(9H-fluoren-9-yl)methoxy]carbonyl}(methyl)amino)-6-(2-methylpropanamido)hexanoic acid, (2S)-2-({[(9H-fluoren-9-yl)methoxy]carbonyl}(methyl)amino)-6-(3-methoxypropanamido)hexanoic acid, (2S)-2-({[(9H-fluoren-9-yl)methoxy]carbonyl}(methyl)amino)-6-[(2S)-2-(triphenylmethoxy)propanamido]hexanoic acid, (2S)-2-({[(9H-fluoren-9-yl)methoxy]carbonyl}(methyl)amino)-6-[(2R)-2-(triphenylmethoxy)propanamido]hexanoic acid, (2S)-2-({[(9H-fluoren-9-yl)methoxy]carbonyl}(methyl)amino)-6-(phenylformamido)hexanoic acid, (2S)-6-[2-(tert-butoxy)acetamido]-2-({[(9H-fluoren-9-yl)methoxy]carbonyl}(methyl)amino)hexanoic acid, (2S)-2-({[(9H-fluoren-9-yl)methoxy]carbonyl}(methyl)amino)-6-(2-methoxyacetamido)hexanoic acid, (2S)-2-({[(9H-fluoren-9-yl)methoxy]carbonyl}(methyl)amino)-6-(morpholin-4-yl)hexanoic acid, (2S)-2-({[(9H-fluoren-9-yl)methoxy]carbonyl}(methyl)amino)-6-methanesulfonamidohexanoic acid, (2S)-2-({[(9H-fluoren-9-yl)methoxy]carbonyl}(methyl)amino)-6-(2-oxoimidazolidin-1-yl)hexanoic acid, (2S)-2-({[(9H-fluoren-9-yl)methoxy]carbonyl}(methyl)amino)-6-(2-oxopyrrolidin-1-yl)hexanoic acid, (2S)-2-({[(9H-fluoren-9-yl)methoxy]carbonyl}(methyl)amino)-6-(2-oxo-1,3-diazinan-1-yl)hexanoic acid, (2S)-3-{4′-cyano-[1,1′-biphenyl]-3-yl}-2-({[(9H-fluoren-9-yl)methoxy]carbonyl}amino)propanoic acid, (2S)-2-({[(9H-fluoren-9-yl)methoxy]carbonyl}(methyl)amino)-6-(1H-1,2,3-triazol-1-yl)hexanoic acid, (2S)-5-acetamido-2-({[(9H-fluoren-9-yl)methoxy]carbonyl}(methyl)amino)pentanoic acid, (2S)-2-({[(9H-fluoren-9-yl)methoxy]carbonyl}amino)-5-methanesulfonamidopentanoic acid, (2S)-2-({[(9H-fluoren-9-yl)methoxy]carbonyl}amino)-4-{[(pyridin-4-yl)methyl]carbamoyl}butanoic acid hydrochloride, (2S)-2-({[(9H-fluoren-9-yl)methoxy]carbonyl}amino)-4-{[(pyridin-4-yl)methyl]carbamoyl}butanoic acid, (2S)-2-({[(9H-fluoren-9-yl)methoxy]carbonyl}amino)-4-{[(1s,3s)-3-{[(tert-butoxy)carbonyl]amino}cyclobutyl]carbamoyl}butanoic acid, (2S)-4-[(carbamoylmethyl)carbamoyl]-2-({[(9H-fluoren-9-yl)methoxy]carbonyl}(methyl)amino)butanoic acid, (2S)-2-({[(9H-fluoren-9-yl)methoxy]carbonyl}amino)-4-{[(pyrimidin-5-yl)methyl]carbamoyl}butanoic acid, (2S)-4-{[(3S)-1-[(tert-butoxy)carbonyl]pyrrolidin-3-yl]carbamoyl}-2-({[(9H-fluoren-9-yl)methoxy]carbonyl}amino)butanoic acid, (2S)-2-({[(9H-fluoren-9-yl)methoxy]carbonyl}amino)-3-(5,6,7,8-tetrahydronaphthalen-2-yl)propanoic acid, (2S)-3-{5-[2-(tert-butoxy)-2-oxoethoxy]-1-[(tert-butoxy)carbonyl]-1H-indol-3-yl}-2-({[(9H-fluoren-9-yl)methoxy]carbonyl}amino)propanoic acid, (2S)-2-({[(9H-fluoren-9-yl)methoxy]carbonyl}amino)-3-{3′-methanesulfonyl-[1,1′-biphenyl]-4-yl}propanoic acid, (2S)-2-({[(9H-fluoren-9-yl)methoxy]carbonyl}amino)-3-methanesulfonamidopropanoic acid, (2S)-3-(2-acetamidoacetamido)-2-({[(9H-fluoren-9-yl)methoxy]carbonyl}(methyl)amino)propanoic acid, (2S)-2-({[(9H-fluoren-9-yl)methoxy]carbonyl}amino)-3-[3-({[(prop-2-en-1-yloxy)carbonyl]amino}methyl)phenyl]propanoic acid, (2S)-2-({[(9H-fluoren-9-yl)methoxy]carbonyl}(methyl)amino)-3-(4-{[(prop-2-en-1-yloxy)carbonyl]amino}phenyl)propanoic acid, (2S)-2-({[(9H-fluoren-9-yl)methoxy]carbonyl}amino)-3-(1,2-oxazol-4-yl)propanoic acid, (2S)-5-(tert-butoxy)-2-({[(9H-fluoren-9-yl)methoxy]carbonyl}(methyl)amino)-5-oxopentanoic acid, (2S)-2-({[(9H-fluoren-9-yl)methoxy]carbonyl}(methyl)amino)-4-[(3-methylpentan-3-yl)oxy]-4-oxobutanoic acid, (2S)-2-({[(9H-fluoren-9-yl)methoxy]carbonyl}(methyl)amino)-3-(triphenylmethoxy)propanoic acid, (2S)-3-(3-chlorophenyl)-2-({[(9H-fluoren-9-yl)methoxy]carbonyl}amino)propanoic acid, (2S)-2-({[(9H-fluoren-9-yl)methoxy]carbonyl}amino)-4-phenylbutanoic acid, (2S)-2-({[(9H-fluoren-9-yl)methoxy]carbonyl}amino)-4-(4-fluorophenyl)butanoic acid, (2S)-4-(4-{[(tert-butoxy)carbonyl]amino}phenyl)-2-({[(9H-fluoren-9-yl)methoxy]carbonyl}amino)butanoic acid, (2S)-2-({[(9H-fluoren-9-yl)methoxy]carbonyl}(methyl)amino)-3-(6-oxo-1,6-dihydropyridin-3-yl)propanoic acid, (2S)-2-({[(9H-fluoren-9-yl)methoxy]carbonyl}(methyl)amino)-3-(4-methoxyphenyl)propanoic acid, (2S)-2-({[(9H-fluoren-9-yl)methoxy]carbonyl}amino)-3-(6-oxo-1,6-dihydropyridin-3-yl)propanoic acid, (2S)-3-(5,6-dimethoxypyridin-3-yl)-2-({[(9H-fluoren-9-yl)methoxy]carbonyl}amino)propanoic acid, (2S)-2-({[(9H-fluoren-9-yl)methoxy]carbonyl}amino)-3-[2-(methylcarbamoyl)pyridin-4-yl]propanoic acid, (2S)-2-({[(9H-fluoren-9-yl)methoxy]carbonyl}amino)-3-(4-methanesulfonyl phenyl)propanoic acid, (2S)-2-({[(9H-fluoren-9-yl)methoxy]carbonyl}(methyl)amino)-3-{4-[(prop-2-en-1-yloxy)carbonyl]phenyl}propanoic acid, (2S)-3-(4-acetamidophenyl)-2-({[(9H-fluoren-9-yl)methoxy]carbonyl}amino)propanoic acid, (2S)-2-({[(9H-fluoren-9-yl)methoxy]carbonyl}amino)-3-(6-methoxypyridin-3-yl)propanoic acid, (2S)-3-{4-[(tert-butoxy)carbonyl]phenyl}-2-({[(9H-fluoren-9-yl)methoxy]carbonyl}(methyl)amino)propanoic acid, (2S)-3-{3-[(tert-butoxy)carbonyl]phenyl}-2-({[(9H-fluoren-9-yl)methoxy]carbonyl}(methyl)amino)propanoic acid, (2S)-3-{5-[(tert-butoxy)carbonyl]pyridin-3-yl}-2-({[(9H-fluoren-9-yl)methoxy]carbonyl}amino)propanoic acid, (2S)-4-{3-[(tert-butoxy)carbonyl]phenyl}-2-({[(9H-fluoren-9-yl)methoxy]carbonyl}amino)butanoic acid, (2S)-2-({[(9H-fluoren-9-yl)methoxy]carbonyl}amino)-3-(2-methoxypyridin-4-yl)propanoic acid, (2S)-3-{3-[(tert-butoxy)carbonyl]-4-hydroxyphenyl}-2-({[(9H-fluoren-9-yl)methoxy]carbonyl}amino)propanoic acid, (2S)-3-[4-(tert-butoxy)-3-methylphenyl]-2-({[(9H-fluoren-9-yl)methoxy]carbonyl}amino)propanoic acid, (2S)-3-(2-{[(tert-butoxy)carbonyl]amino}pyridin-4-yl)-2-({[(9H-fluoren-9-yl)methoxy]carbonyl}(methyl)amino)propanoic acid, (2S)-3-[4-({[(tert-butoxy)carbonyl]amino}methyl)phenyl]-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-[4-({[(prop-2-en-1-yloxy)carbonyl]amino}methyl)phenyl]propanoic acid, (2S)-3-{3-[(3-{[(tert-butoxy)carbonyl]amino}propyl)carbamoyl]phenyl}-2-({[(9H-fluoren-9-yl)methoxy]carbonyl}(methyl)amino)propanoic acid, (2S)-3-{3-[(2-{[(tert-butoxy)carbonyl]amino}ethyl)carbamoyl]phenyl}-2-({[(9H-fluoren-9-yl)methoxy]carbonyl}(methyl)amino)propanoic acid, (2S)-3-(6-{[(tert-butoxy)carbonyl](methyl)amino}pyridin-3-yl)-2-({[(9H-fluoren-9-yl)methoxy]carbonyl}amino)propanoic acid, (2S)-3-[3-({[(tert-butoxy)carbonyl]amino}methyl)phenyl]-2-({[(9H-fluoren-9-yl)methoxy]carbonyl}(methyl)amino)propanoic acid, (2S,4R)-4-(tert-butoxy)-1-{[(9H-fluoren-9-yl)methoxy]carbonyl}pyrrolidine-2-carboxylic acid, (2R,4S)-4-{[(tert-butoxy)carbonyl]amino}-1-{[(9H-fluoren-9-yl)methoxy]carbonyl}pyrrolidine-2-carboxylic acid, (2R,4R)-1-{[(9H-fluoren-9-yl)methoxy]carbonyl}-4-hydroxypyrrolidine-2-carboxylic acid, (2S)-2-({[(9H-fluoren-9-yl)methoxy]carbonyl}(methyl)amino)pentanoic acid, (2S)-3-cyclobutyl-2-({[(9H-fluoren-9-yl)methoxy]carbonyl}amino)propanoic acid, (2S)-3-cyclopentyl-2-({[(9H-fluoren-9-yl)methoxy]carbonyl}amino)propanoic acid, (2S)-4-ethyl-2-({[(9H-fluoren-9-yl)methoxy]carbonyl}amino)hexanoic acid, (2S)-2-({[(9H-fluoren-9-yl)methoxy]carbonyl}amino)-7,7,7-trifluoroheptanoic acid, (2S)-4-cyclobutyl-2-({[(9H-fluoren-9-yl)methoxy]carbonyl}amino)butanoic acid, (2S)-4-cyclopentyl-2-({[(9H-fluoren-9-yl)methoxy]carbonyl}amino)butanoic acid, (2S)-2-({[(9H-fluoren-9-yl)methoxy]carbonyl}amino)-3-(1-methyl-1H-pyrazol-4-yl)propanoic acid, (2S)-2-({[(9H-fluoren-9-yl)methoxy]carbonyl}amino)-3-(1,3-oxazol-5-yl)propanoic acid, (2S)-2-({[(9H-fluoren-9-yl)methoxy]carbonyl}amino)-3-(1,3-oxazol-4-yl)propanoic acid, (2S)-2-({[(9H-fluoren-9-yl)methoxy]carbonyl}amino)-3-[1-(triphenylmethyl)-1H-pyrazol-4-yl]propanoic acid, (2S)-2-({[(9H-fluoren-9-yl)methoxy]carbonyl}amino)-3-(5-hydroxy-1H-indol-3-yl)propanoic acid, (2S)-2-({[(9H-fluoren-9-yl)methoxy]carbonyl}amino)-3-(naphthalen-1-yl)propanoic acid, (2S)-2-({[(9H-fluoren-9-yl)methoxy]carbonyl}(methyl)amino)-3-(naphthalen-2-yl)propanoic acid, (2S)-3-{1-[(tert-butoxy)carbonyl]-7-methoxy-1H-indol-3-yl}-2-({[(9H-fluoren-9-yl)methoxy]carbonyl}amino)propanoic acid, (2S)-2-({[(9H-fluoren-9-yl)methoxy]carbonyl}amino)-3-(quinolin-6-yl)propanoic acid, (2S)-3-{1-[(tert-butoxy)carbonyl]-5-hydroxy-1H-indol-3-yl}-2-({[(9H-fluoren-9-yl)methoxy]carbonyl}amino)propanoic acid, (2S)-2-({[(9H-fluoren-9-yl)methoxy]carbonyl}amino)-3-{1H-pyrrolo[3,2-c]pyridin-3-yl}propanoic acid, (2S)-2-({[(9H-fluoren-9-yl)methoxy]carbonyl}amino)-3-{1-methyl-1H-pyrrolo[2,3-b]pyridin-3-yl}propanoic acid, (2S)-3-(1-{2-[(4-{[1-(4,4-dimethyl-2,6-dioxocyclohexylidene)-3-methylbutyl]amino}phenyl)methoxy]-2-oxoethyl}-1H-indol-3-yl)-2-({[(9H-fluoren-9-yl)methoxy]carbonyl}amino)propanoic acid, (2S)-2-({[(9H-fluoren-9-yl)methoxy]carbonyl}(methyl)amino)-3-(1H-indazol-5-yl)propanoic acid, (2S)-3-[5-(benzyloxy)-1H-indol-3-yl]-2-({[(9H-fluoren-9-yl)methoxy]carbonyl}amino)propanoic acid, (2S)-3-{1-[(tert-butoxy)carbonyl]-5-[(4-methoxyphenyl)methoxy]-1H-indol-3-yl}-2-({[(9H-fluoren-9-yl)methoxy]carbonyl}amino)propanoic acid, (2S,3S)-3-(tert-butoxy)-2-({[(9H-fluoren-9-yl)methoxy]carbonyl}amino)butanoic acid, (2S)-2-({[(9H-fluoren-9-yl)methoxy]carbonyl}amino)-3-(oxan-4-yl)propanoic acid, (2S)-2-({[(9H-fluoren-9-yl)methoxy]carbonyl}amino)-3-[(1r,4r)-4-methoxycyclohexyl]propanoic acid, (2S)-2-({[(9H-fluoren-9-yl)methoxy]carbonyl}amino)-3-[(1r,4r)-4-hydroxycyclohexyl]propanoic acid, (2S)-3-butoxy-2-({[(9H-fluoren-9-yl)methoxy]carbonyl}amino)propanoic acid, (2S)-2-({[(9H-fluoren-9-yl)methoxy]carbonyl}amino)-3-(pentyloxy)propanoic acid, (2S)-2-({[(9H-fluoren-9-yl)methoxy]carbonyl}amino)-4-propoxybutanoic acid, (2S)-4-ethoxy-2-({[(9H-fluoren-9-yl)methoxy]carbonyl}amino)butanoic acid, (2S)-4-butoxy-2-({[(9H-fluoren-9-yl)methoxy]carbonyl}amino)butanoic acid, (2S)-3-(cyclopentyloxy)-2-({[(9H-fluoren-9-yl)methoxy]carbonyl}amino)propanoic acid, (2S)-3-(cyclobutylmethoxy)-2-({[(9H-fluoren-9-yl)methoxy]carbonyl}amino)propanoic acid, (2S)-4-(cyclopentyloxy)-2-({[(9H-fluoren-9-yl)methoxy]carbonyl}amino)butanoic acid, (2S)-2-({[(9H-fluoren-9-yl)methoxy]carbonyl}amino)-4-(propan-2-yloxy)butanoic acid, (2S)-2-({[(9H-fluoren-9-yl)methoxy]carbonyl}amino)-3-[methyl(phenyl)amino]propanoic acid hydrochloride, (2S)-4-acetamido-2-({[(9H-fluoren-9-yl)methoxy]carbonyl}amino)butanoic acid, (2R)-3-{1-[(tert-butoxy)carbonyl]piperidin-4-yl}-2-({[(9H-fluoren-9-yl)methoxy]carbonyl}amino)propanoic acid, (2R)-4-{[(tert-butoxy)carbonyl]amino}-2-({[(9H-fluoren-9-yl)methoxy]carbonyl}amino)butanoic acid, (2R)-7-{[(tert-butoxy)carbonyl]amino}-2-({[(9H-fluoren-9-yl)methoxy]carbonyl}amino)heptanoic acid, (2S)-2-({[(9H-fluoren-9-yl)methoxy]carbonyl}amino)-7-{[(prop-2-en-1-yloxy)carbonyl]amino}heptanoic acid, (2R)-2-({[(9H-fluoren-9-yl)methoxy]carbonyl}amino)-7-{[(prop-2-en-1-yloxy)carbonyl]amino}heptanoic acid, (2S)-2-({[(9H-fluoren-9-yl)methoxy]carbonyl}amino)-3-(1-methanesulfonylpiperidin-4-yl)propanoic acid, (2R)-5-(carbamoylamino)-2-({[(9H-fluoren-9-yl)methoxy]carbonyl}amino)pentanoic acid, (2S)-6-(carbamoylamino)-2-({[(9H-fluoren-9-yl)methoxy]carbonyl}amino)hexanoic acid, (2S)-2-({[(9H-fluoren-9-yl)methoxy]carbonyl}(methyl)amino)-6-oxo-6-(prop-2-en-1-yloxy)hexanoic acid, (2S)-6-(tert-butoxy)-2-({[(9H-fluoren-9-yl)methoxy]carbonyl}(methyl)amino)-6-oxohexanoic acid, (2S)-5-(dimethylcarbamoyl)-2-({[(9H-fluoren-9-yl)methoxy]carbonyl}(methyl)amino)pentanoic acid, (2R)-2-({[(9H-fluoren-9-yl)methoxy]carbonyl}(methyl)amino)-4-[(3-methylpentan-3-yl)oxy]-4-oxobutanoic acid, (2S)-3-(3-carbamoylphenyl)-2-({[(9H-fluoren-9-yl)methoxy]carbonyl}amino)propanoic acid, (2S)-3-(4-carbamoylphenyl)-2-({[(9H-fluoren-9-yl)methoxy]carbonyl}amino)propanoic acid, (2R)-3-(4-carbamoylphenyl)-2-({[(9H-fluoren-9-yl)methoxy]carbonyl}(methyl)amino)propanoic acid, (2R)-3-(3-carbamoylphenyl)-2-({[(9H-fluoren-9-yl)methoxy]carbonyl}(methyl)amino)propanoic acid, (2S)-3-(4-{[(tert-butoxy)carbonyl]amino}phenyl)-2-({[(9H-fluoren-9-yl)methoxy]carbonyl}(methyl)amino)propanoic acid, (2R)-2-({[(9H-fluoren-9-yl)methoxy]carbonyl}amino)-3-[3-({[(prop-2-en-1-yloxy)carbonyl]amino}methyl)phenyl]propanoic acid, (2S)-3-[4-(acetamidomethyl)phenyl]-2-({[(9H-fluoren-9-yl)methoxy]carbonyl}(methyl)amino)propanoic acid, 2-({[(9H-fluoren-9-yl)methoxy]carbonyl}[2-(N-methylmethanesulfonamido)ethyl]amino)acetic acid, (2S)-4-(carbamoylamino)-2-({[(9H-fluoren-9-yl)methoxy]carbonyl}amino)butanoic acid, (2S)-5-(cyclohexylformamido)-2-({[(9H-fluoren-9-yl)methoxy]carbonyl}amino)pentanoic acid, (2S)-3-{3-[(3-{[(tert-butoxy)carbonyl]amino}propyl)carbamoyl]phenyl}-2-({[(9H-fluoren-9-yl)methoxy]carbonyl}amino)propanoic acid, (2S)-3-(7-chloro-1-methyl-1H-indol-3-yl)-2-({[(9H-fluoren-9-yl)methoxy]carbonyl}amino)propanoic acid, (2S)-2-({[(9H-fluoren-9-yl)methoxy]carbonyl}amino)-3-[4-(1-methyl-1H-pyrazol-4-yl)phenyl]propanoic acid, (2S)-2-({[(9H-fluoren-9-yl)methoxy]carbonyl}amino)-3-[4-(pyridin-3-yl)phenyl]propanoic acid hydrochloride, (2S)-2-({[(9H-fluoren-9-yl)methoxy]carbonyl}(methyl)amino)-3-(isoquinolin-7-yl)propanoic acid, (2S)-2-({[(9H-fluoren-9-yl)methoxy]carbonyl}amino)-3-(2-phenylpyridin-4-yl)propanoic acid, (2S)-2-({[(9H-fluoren-9-yl)methoxy]carbonyl}amino)-3-(quinolin-5-yl)propanoic acid, (2S)-3-{3-[(2-{[(tert-butoxy)carbonyl]amino}ethyl)carbamoyl]phenyl}-2-({[(9H-fluoren-9-yl)methoxy]carbonyl}amino)propanoic acid, 2-({[(9H-fluoren-9-yl)methoxy]carbonyl}({2-[(triphenylmethyl)carbamoyl]ethyl})amino)acetic acid, (2S)-2-({[(9H-fluoren-9-yl)methoxy]carbonyl}amino)-3-[4-(pyridin-2-yl)phenyl]propanoic acid hydrochloride, (2S)-4-[(2-carbamoylethyl)carbamoyl]-2-({[(9H-fluoren-9-yl)methoxy]carbonyl}amino)butanoic acid, (2S)-3-[4-(1-acetylpiperidin-4-yl)phenyl]-2-({[(9H-fluoren-9-yl)methoxy]carbonyl}amino)propanoic acid, (2S)-2-({[(9H-fluoren-9-yl)methoxy]carbonyl}(methyl)amino)-3-(quinolin-7-yl)propanoic acid, (2S)-2-({[(9H-fluoren-9-yl)methoxy]carbonyl}amino)-4-oxo-4-(pyrrolidin-1-yl)butanoic acid, (2S)-3-[1-(3-{[(tert-butoxy)carbonyl]amino}propyl)-1H-indol-3-yl]-2-({[(9H-fluoren-9-yl)methoxy]carbonyl}amino)propanoic acid, 2-[(2-{[1,1′-biphenyl]-4-yl}ethyl)({[(9H-fluoren-9-yl)methoxy]carbonyl})amino]acetic acid, (2S)-3-{1-[(tert-butoxy)carbonyl]-5,6-dichloro-1H-indol-3-yl}-2-({[(9H-fluoren-9-yl)methoxy]carbonyl}amino)propanoic acid, (2S)-3-(4-{1-[(tert-butoxy)carbonyl]piperidin-4-yl}phenyl)-2-({[(9H-fluoren-9-yl)methoxy]carbonyl}amino)propanoic acid, (2S)-2-({[(9H-fluoren-9-yl)methoxy]carbonyl}amino)-3-(4-{1-[(prop-2-en-1-yloxy)carbonyl]piperidin-4-yl}phenyl)propanoic acid, (2S)-3-(6-carbamoylpyridin-3-yl)-2-({[(9H-fluoren-9-yl)methoxy]carbonyl}amino)propanoic acid, (2S)-3-{1-[(tert-butoxy)carbonyl]-1H-pyrrolo[3,2-b]pyridin-3-yl}-2-({[(9H-fluoren-9-yl)methoxy]carbonyl}amino)propanoic acid, (2R)-2-({[(9H-fluoren-9-yl)methoxy]carbonyl}amino)-3-{3-[(prop-2-en-1-yloxy)carbonyl]phenyl}propanoic acid, (2R)-3-{4-[(tert-butoxy)carbonyl]phenyl}-2-({[(9H-fluoren-9-yl)methoxy]carbonyl}amino)propanoic acid, (2R)-2-({[(9H-fluoren-9-yl)methoxy]carbonyl}amino)-3-{4-[(prop-2-en-1-yloxy)carbonyl]phenyl}propanoic acid, (2S)-2-({[(9H-fluoren-9-yl)methoxy]carbonyl}amino)-3-[5-methyl-1-(triphenylmethyl)-1H-imidazol-4-yl]propanoic acid, (2R)-3-{[(2,4-dimethoxyphenyl)methyl]sulfamoyl}-2-({[(9H-fluoren-9-yl)methoxy]carbonyl}amino)propanoic acid, (S)-2-((((9H-fluoren-9-yl)methoxy)carbonyl)amino)-3-(5-phenylpyridin-3-yl)propanoic acid, (S)-2-((((9H-fluoren-9-yl)methoxy)carbonyl)(methyl)amino)-3-(3,4-dichlorophenyl)propanoic acid, (S)-2-((((9H-fluoren-9-yl)methoxy)carbonyl)amino)-3-(4-(2-amino-2-oxoethyl)phenyl)propanoic acid, (S)-2-((((9H-fluoren-9-yl)methoxy)carbonyl)amino)-3-(2-carbamoylpyridin-3-yl)propanoic acid, (S)-2-((((9H-fluoren-9-yl)methoxy)carbonyl)amino)-3-(5,6-dimethylpyridin-3-yl)propanoic acid, (S)-2-((((9H-fluoren-9-yl)methoxy)carbonyl)amino)-3-(2,6-dimethylpyridin-4-yl)propanoic acid, (R)-2-((((9H-fluoren-9-yl)methoxy)carbonyl)amino)-3-(2-((tert-butoxycarbonyl)amino)pyridin-4-yl)propanoic acid, (S)-2-((((9H-fluoren-9-yl)methoxy)carbonyl)amino)-4-(5-(trifluoromethyl)pyridin-3-yl)butanoic acid, (S)-2-((((9H-fluoren-9-yl)methoxy)carbonyl)amino)-4-(2-(trifluoromethyl)pyridin-4-yl)butanoic acid, (R)-2-((((9H-fluoren-9-yl)methoxy)carbonyl)(methyl)amino)-3-cyclobutylpropanoic acid, (S)-2-((((9H-fluoren-9-yl)methoxy)carbonyl)(methyl)amino)-3-(2,3-dichlorophenyl)propanoic acid, (S)-2-((((9H-fluoren-9-yl)methoxy)carbonyl)amino)-3-(1-trityl-1,4,5,6-tetrahydrocyclopenta[c]pyrazol-3-yl)propanoic acid, (S)-2-((((9H-fluoren-9-yl)methoxy)carbonyl)amino)-3-(4-(tetrahydro-2H-pyran-4-yl)phenyl)propanoic acid, (S)-2-((((9H-fluoren-9-yl)methoxy)carbonyl)amino)-4-(3-(pentafluoro-16-sulfaneyl)phenyl)butanoic acid, (2S,4R)-1-(((9H-fluoren-9-yl)methoxy)carbonyl)-4-(2-chlorophenoxy)pyrrolidine-2-carboxylic acid, (2S,4R)-1-(((9H-fluoren-9-yl)methoxy)carbonyl)-4-(2-fluorophenoxy)pyrrolidine-2-carboxylic acid, (2S,4R)-1-(((9H-fluoren-9-yl)methoxy)carbonyl)-4-(3-chlorophenoxy)pyrrolidine-2-carboxylic acid, (2S,4R)-1-(((9H-fluoren-9-yl)methoxy)carbonyl)-4-(3-fluorophenoxy)pyrrolidine-2-carboxylic acid, (2S,4R)-1-(((9H-fluoren-9-yl)methoxy)carbonyl)-4-(4-chlorophenoxy)pyrrolidine-2-carboxylic acid, (2S,4R)-1-(((9H-fluoren-9-yl)methoxy)carbonyl)-4-(4-fluorophenoxy)pyrrolidine-2-carboxylic acid, (2S)-2-({[(9H-fluoren-9-yl)methoxy]carbonyl}amino)-3-{2-fluoro-[1,1′-biphenyl]-4-yl}propanoic acid, (S)-2-((((9H-fluoren-9-yl)methoxy)carbonyl)amino)-3-(6-(2-((tert-butoxycarbonyl)amino)ethoxy)pyridin-3-yl)propanoic acid, (S)-2-((((9H-fluoren-9-yl)methoxy)carbonyl)amino)-4-(dimethylamino)butanoic acid hydrochloride, (S)-2-((((9H-fluoren-9-yl)methoxy)carbonyl)amino)-3-(4-(quinolin-4-yl)phenyl)propanoic acid, (S,Z)-2-((((9H-fluoren-9-yl)methoxy)carbonyl)amino)-3-(4-(2-methyl-3-((2,2,4,6,7-pentamethyl-2,3-dihydrobenzofuran-5-yl)sulfonyl)guanidino)phenyl)propanoic acid, (S)-2-((((9H-fluoren-9-yl)methoxy)carbonyl)amino)-3-(4-((1-((2,2,4,6,7-pentamethyl-2,3-dihydrobenzofuran-5-yl)sulfonyl)-4,5-dihydro-1H-imidazol-2-yl)amino)phenyl)propanoic acid, (S)-2-((((9H-fluoren-9-yl)methoxy)carbonyl)amino)-3-(2-(dimethylcarbamoyl)-1-((2-(trimethylsilyl)ethoxy)methyl)-1H-imidazol-4-yl)propanoic acid, (S)-2-((((9H-fluoren-9-yl)methoxy)carbonyl)amino)-3-(2-(methylcarbamoyl)-1-((2-(trimethylsilyl)ethoxy)methyl)-1H-imidazol-4-yl)propanoic acid, (2S)-3-[({1-[(tert-butoxy)carbonyl]azetidin-3-yl}methyl)carbamoyl]-2-({[(9H-fluoren-9-yl)methoxy]carbonyl}amino)propanoic acid, (S)-2-((((9H-fluoren-9-yl)methoxy)carbonyl)amino)-3-(isoquinolin-8-yl)propanoic acid, (2S,4R)-1-(((9H-fluoren-9-yl)methoxy)carbonyl)-4-(pyridin-2-yloxy)pyrrolidine-2-carboxylic acid, (2S,4R)-1-(((9H-fluoren-9-yl)methoxy)carbonyl)-4-(pyridin-3-yloxy)pyrrolidine-2-carboxylic acid, (S)-2-((((9H-fluoren-9-yl)methoxy)carbonyl)(methyl)amino)-3-(thiazol-2-yl)propanoic acid, (S)-2-((((9H-fluoren-9-yl)methoxy)carbonyl)(methyl)amino)-3-(5-chloropyridin-3-yl)propanoic acid, (S)-2-((((9H-fluoren-9-yl)methoxy)carbonyl)(methyl)amino)-3-(5-(trifluoromethyl)pyridin-3-yl)propanoic acid, (2S)-3-(2,6-dichloropyridin-4-yl)-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-(oxan-4-yl)propanoic acid, (2R)-2-({[(9H-fluoren-9-yl)methoxy]carbonyl}(methyl)amino)-3-{1-[(prop-2-en-1-yloxy)carbonyl]piperidin-4-yl}propanoic acid, (2R)-2-({[(9H-fluoren-9-yl)methoxy]carbonyl}(methyl)amino)-3-(oxan-4-yl)propanoic acid, (2R)-6-amino-2-({[(9H-fluoren-9-yl)methoxy]carbonyl}(methyl)amino)hexanoic acid hydrochloride, (2R)-2-({[(9H-fluoren-9-yl)methoxy]carbonyl}(methyl)amino)-6-{[(prop-2-en-1-yloxy)carbonyl]amino}hexanoic acid, (2S)-3-(2,3-dimethylphenyl)-2-({[(9H-fluoren-9-yl)methoxy]carbonyl}(methyl)amino)propanoic acid, (2S)-3-[3,5-bis(trifluoromethyl)phenyl]-2-({[(9H-fluoren-9-yl)methoxy]carbonyl}(methyl)amino)propanoic acid, (2S)-3-[3-chloro-5-(trifluoromethyl)phenyl]-2-({[(9H-fluoren-9-yl)methoxy]carbonyl}(methyl)amino)propanoic acid, (2S)-3-(3-ethylphenyl)-2-({[(9H-fluoren-9-yl)methoxy]carbonyl}(methyl)amino)propanoic acid, (2S)-2-({[(9H-fluoren-9-yl)methoxy]carbonyl}(methyl)amino)-6,6,6-trifluorohexanoic acid, (2S)-5-{[(tert-butoxy)carbonyl](methyl)amino}-2-({[(9H-fluoren-9-yl)methoxy]carbonyl}(methyl)amino)pentanoic acid, (2S)-2-({[(9H-fluoren-9-yl)methoxy]carbonyl}(methyl)amino)-5-{methyl[(prop-2-en-1-yloxy)carbonyl]amino}pentanoic acid, (2S)-4-(cyclohexyloxy)-2-({[(9H-fluoren-9-yl)methoxy]carbonyl}amino)butanoic acid, (2S)-2-({[(9H-fluoren-9-yl)methoxy]carbonyl}amino)-4-(oxan-4-yloxy)butanoic acid (2S)-3-(2,2-dimethylpropoxy)-2-({[(9H-fluoren-9-yl)methoxy]carbonyl}amino)propanoic acid, (2S)-2-({[(9H-fluoren-9-yl)methoxy]carbonyl}(methyl)amino)-3-[2-oxo-2-(prop-2-en-1-yloxy)ethoxy]propanoic acid, (2R)-3-(3,4-dichlorophenyl)-2-({[(9H-fluoren-9-yl)methoxy]carbonyl}(methyl)amino)propanoic acid, (2R)-2-({[(9H-fluoren-9-yl)methoxy]carbonyl}amino)-3-(6-oxo-1,6-dihydropyridin-3-yl)propanoic acid, (2R)-4-(4-{[(tert-butoxy)carbonyl]amino}phenyl)-2-({[(9H-fluoren-9-yl)methoxy]carbonyl}amino)butanoic acid, (2R)-2-({[(9H-fluoren-9-yl)methoxy]carbonyl}(methyl)amino)-3-(6-methoxypyridin-3-yl)propanoic acid, (2S)-2-({[(9H-fluoren-9-yl)methoxy]carbonyl}(methyl)amino)-3-(2-methoxypyridin-4-yl)propanoic acid, (2R)-2-({[(9H-fluoren-9-yl)methoxy]carbonyl}(methyl)amino)-3-(6-oxo-1,6-dihydropyridin-3-yl)propanoic acid, (2S)-3-{3-[(tert-butoxy)carbonyl]-4-hydroxyphenyl}-2-({[(9H-fluoren-9-yl)methoxy]carbonyl}(methyl)amino)propanoic acid, (2S)-2-({[(9H-fluoren-9-yl)methoxy]carbonyl}(methyl)amino)-4-(4-fluorophenyl)butanoic acid, (2S)-2-({[(9H-fluoren-9-yl)methoxy]carbonyl}(methyl)amino)-3-(1,3-oxazol-4-yl)propanoic acid, (2S)-2-({[(9H-fluoren-9-yl)methoxy]carbonyl}(methyl)amino)-3-(1-methyl-1H-pyrazol-4-yl)propanoic acid, (2R)-2-({[(9H-fluoren-9-yl)methoxy]carbonyl}amino)-3-(5-phenylpyridin-3-yl)propanoic acid, (2R)-2-({[(9H-fluoren-9-yl)methoxy]carbonyl}amino)-3-(2-phenylpyridin-4-yl)propanoic acid, (2S)-2-({[(9H-fluoren-9-yl)methoxy]carbonyl}(methyl)amino)-3-(quinolin-6-yl)propanoic acid, (2R)-2-({[(9H-fluoren-9-yl)methoxy]carbonyl}amino)-3-(quinolin-5-yl)propanoic acid, (2S)-2-({[(9H-fluoren-9-yl)methoxy]carbonyl}amino)-3-(isoquinolin-7-yl)propanoic acid, (2S)-2-({[(9H-fluoren-9-yl)methoxy]carbonyl}(methyl)amino)-3-(isoquinolin-8-yl)propanoic acid, (2R,4R)-4-{[(tert-butoxy)carbonyl]amino}-1-{[(9H-fluoren-9-yl)methoxy]carbonyl}pyrrolidine-2-carboxylic acid, (2S)-4-ethoxy-2-({[(9H-fluoren-9-yl)methoxy]carbonyl}(methyl)amino)butanoic acid, (2S)-3-butoxy-2-({[(9H-fluoren-9-yl)methoxy]carbonyl}(methyl)amino)propanoic acid, (2S)-3-{[(2,4-dimethoxyphenyl)methyl]sulfamoyl}-2-({[(9H-fluoren-9-yl)methoxy]carbonyl}amino)propanoic acid, (2S)-2-({[(9H-fluoren-9-yl)methoxy]carbonyl}(methyl)amino)-4-propoxybutanoic acid, (2S)-2-({[(9H-fluoren-9-yl)methoxy]carbonyl}amino)-3-[4-({[(prop-2-en-1-yloxy)carbonyl]amino}methyl)phenyl]propanoic acid and (2S)-2-amino-7-{[4-(carboxymethyl)piperazine-1-carbonyl]amino}heptanoic acid.

### <Exemplary Description of Synthesizing the Amino Acid Derivatives>

Examples of synthesizing the novel amino acids in this inventions shall be described below. Further, novel amino acids in this invention can be synthesized by the chemical synthetic method below, other known methods or a combination thereof.

### Synthetic Method;

using Negishi coupling reaction (Organic & Biomolecular Chemistry (2003), 1(23), 4254-4261);
using Suzuki coupling reaction (Journal of Organic Chemistry (2016), 81(19), 9499-9506);
using Chiral-Template reaction (Symmetry (2019), 11(4), 578, Bioorganic & Medicinal Chemistry Letters (2010), 20(1), 236-239, JP2009096791 A);
using Asymmetric alkylation by Maruoka Catalyst (trademark registered) (Journal of the American Chemical Society (2000), 122(21), 5228-5229, WO2015076346 A1);
Via cyclic sulfamidate derivate (WO2020095983 A1, Bioorganic Chemistry (2020), 100, 103864); or
Methylation method of main chain in the amino acid (Journal of Organic Chemistry (2003), 68(7), 2652-2667).
An example of a synthesis method is shown below.

### Reference Example 1-1-01: Prop-2-en-1-yl (2S)-6-amino-2-({[(9H-fluoren-9-yl)methoxy]carbonyl}(methyl)amino)hexanoate hydrochloride

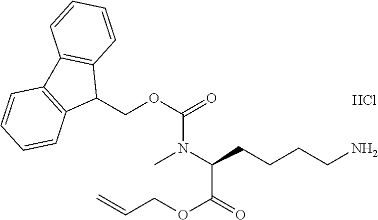 [SMILES: C=CCOC(=O)[C@H](CCCCN)N(C)C(=O)OCC1c2ccccc2-c2ccccc21 ; Cl]

To a solution of (2S)-2-[9H-fluoren-9-ylmethoxycarbonyl(methyl)amino]-6-[(2-methylpropan-2-yl)oxycarbonylamino]hexanoic acid (CAS: 197632-76-1, 12 g, 24.9 mmol) in DMF (99 mL) at 25° C. were added DIPEA (6.51 mL, 37.3 mmol) and allyl bromide (2.37 mL, 27.4 mmol). The mixture was stirred at 25° C. for ca. 16 h. The reaction was quenched with H₂O. The mixture was extracted with EtOAc. The organic layer was dried over Na₂SO₄, filtered and concentrated. The resulting residue was purified by silica gel flash column chromatography (gradient, hexane/EtOAc=100/0 to 50/50). Appropriate fractions were concentrated. A mixture of the resulting residue (12.1 g, 22.92 mmol) and and 2M HCl in DCM/CPME (ca. 1/1, 232 mL) was stirred at 25° C. for 2 h. The mixture was concentrated to furnish the title compound (10.35 g, 22.32 mmol) which was used in the next step without any further purification. MS (ESI+): m/z 423.4 [M+H].

### Reference Example 1-1

The following compounds were synthesized as outlined for the preparation of Reference Example-1-1-01 employing appropriate starting materials in the table.

| | Structure and IUPAC name |
|---|---|
| Ex- | Starting material acid |
| ample | MS data |
| 1-1-02 | 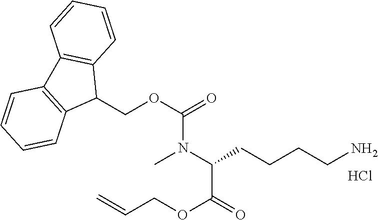 [SMILES: C=CCOC(=O)[C@@H](CCCCN)N(C)C(=O)OCC1c2ccccc2-c2ccccc21 ; Cl] |
| | prop-2-en-1-yl (2R)-6-amino-2-({[(9H- |
| | fluoren-9-yl)methoxy]carbonyl} |
| | (methyl)amino)hexanoate hydrochloride |
| | (2R)-6-[[(tert-butoxy)carbonyl]amino]- |
| | 2-([[(9H-fluoren-9-yl)methoxy] |
| | carbonyl](methyl)amino)hexanoic acid (CAS: 1793105-27-7) |
| | MS (ESI+): m/z 423.3 [M + H] |
| 1-1-03 | 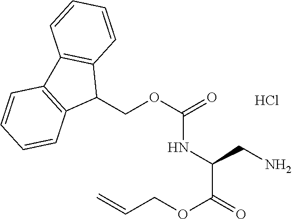 [SMILES: C=CCOC(=O)[C@H](CN)NC(=O)OCC1c2ccccc2-c2ccccc21 ; Cl] |
| | prop-2-en-1-yl (2S)-3-amino-2-({[(9H- |
| | fluoren-9-yl)methoxy]carbonyl} |
| | amino)propanoate hydrochloride |
| | (2S)-3-{[(tert-butoxy)carbonyl]amino}- |
| | 2-({[(9H-fluoren-9-yl)methoxy] |
| | carbonyl}amino)propanoic acid (CAS: 162558-25-0) |
| | MS (ESI+): m/z 367.3 [M + H] |
| 1-1-04 | 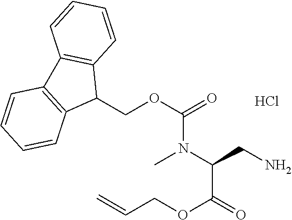 [SMILES: C=CCOC(=O)[C@H](CN)N(C)C(=O)OCC1c2ccccc2-c2ccccc21 ; Cl] |
| | prop-2-en-1-yl (2S)-3-amino-2-({[(9H- |
| | fluoren-9-yl)methoxy]carbonyl} |
| | (methyl)amino)propanoate hydrochloride |
| | (2S)-3-{[(tert-butoxy)carbonyl]amino}- |
| | 2-({[(9H-fluoren-9-yl)methoxy] |
| | carbonyl}(methyl)amino)propanoic acid |
| | MS (ESI+): m/z 381.4 [M + H] |
| 1-1-05 | 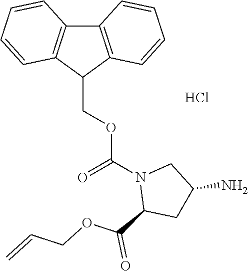 [SMILES: C=CCOC(=O)[C@@H]1C[C@@H](N)CN1C(=O)OCC1c2ccccc2-c2ccccc21 ; Cl] |
| | 1-(9H-fluoren-9-yl)methyl 2-prop-2-en-1-yl (2S,4R)-4- |
| | aminopyrrolidine-1,2-dicarboxylate hydrochloride |
| | (2S,4R)-4-{[(tert-butoxy)carbonyl]amino}-1- |
| | {[(9H-fluoren-9-yl)methoxy]carbonyl} |
| | pyrrolidine-2-carboxylic acid (CAS: 273222-06-3) |
| | MS (ESI+): m/z 393.4 [M + H] |
| 1-1-06 | 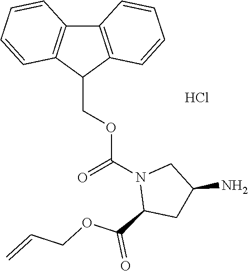 [SMILES: C=CCOC(=O)[C@@H]1C[C@H](N)CN1C(=O)OCC1c2ccccc2-c2ccccc21 ; Cl] |
| | 1-(9H-fluoren-9-yl)methyl 2-prop-2-en-1-yl (2S,4S)-4- |
| | aminopyrrolidine-1,2-dicarboxylate hydrochloride |
| | (2S,4S)-4-{[(tert-butoxy)carbonyl]amino}- |
| | 1-{[(9H-fluoren-9-yl)methoxy]carbonyl} |
| | pyrrolidine-2-carboxylic acid (CAS: 221352-74-5) |
| | MS (ESI+): m/z 393.4 [M + H] |

Following compounds were prepared and used for preparations of the disclosure.

### Intermediate 1-1-01. tert-butyl 4-((((2,5-dioxopyrrolidin-1-yl)oxy)carbonyl)amino)piperidine-1-carboxylate

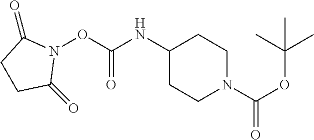 [SMILES: CC(C)(C)OC(=O)N1CCC(NC(=O)ON2C(=O)CCC2=O)CC1]

To a suspension of N,N′-disuccinimidyl carbonate (CAS: 74124-79-1, 5.12 g, 19.97 mmol) in CH₂Cl₂ (33 mL) at 0° C. was added tert-butyl 4-aminopiperidine-1-carboxylate (CAS: 87120-72-7, 2 g, 9.99 mmol). The mixture was stirred at 0° C. for 0.25 h, and then concentrated. The resulting residue was purified by silica gel flash column chromatography (gradient, hexane to hexane/EtOAc) to afford the title compound (3.05 g, 8.93 mmol). MS (ESI+): m/z 286.2 (M−tBu+H).

### Intermediate 1-1-02. tert-butyl 4-((((2,5-dioxopyrrolidin-1-yl)oxy)carbonyl)oxy)piperidine-1-carboxylate

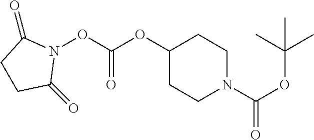 [SMILES: CC(C)(C)OC(=O)N1CCC(OC(=O)ON2C(=O)CCC2=O)CC1]

To a suspension of N,N′-disuccinimidyl carbonate (5.09 g, 19.87 mmol) and DIPEA (2.60 mL, 14.91 mmol) in acetonitrile (33 mL) at 0° C. was added tert-butyl 4-hydroxypiperidine-1-carboxylate (2.0 g, 9.94 mmol). The mixture was stirred at 0° C. for 0.25 h, and then concentrated. The resulting residue was purified by silica gel flash column chromatography (gradient, hexane/EtOAc=100/0 to 80/20) to afford the title compound (2.72 g, 7.95 mmol). MS (ESI+): m/z 287.2 (M−tBu+H).

### Intermediate-1-1-03: 2-phenylpropan-2-yl 2-(piperazin-1-yl)acetate

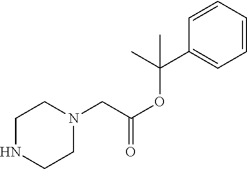 [SMILES: CC(C)(OC(=O)CN1CCNCC1)c1ccccc1]

To a refluxed solution of piperazine (7.42 g, 86 mmol) in THF (48 mL) was added dropwise a solution of 2-phenylpropan-2-yl 2-bromoacetate (CAS: 153698-47-6, 3.69 g, 14.35 mmol) in THF (24 mL) over 5 min. The mixture was stirred for 2 h under the reflux, and then cooled to rt, and then filtered. The filtrate was concentrated. The resulting residue was purified by silica gel flash column chromatography (gradient, CH₂Cl₂/MeOH=100/0 to 80/20, and then CH₂Cl₂/MeOH with 1% Et₃N=100/0 to 80/20) to afford the title compound (2.57 g, 9.81 mmol). ¹H NMR (500 MHz, DMSO-d₆) δ 7.37-7.30 (m, 4H), 7.28-7.20 (m, 1H), 3.13 (br s, 2H), 2.67-2.61 (m, 4H), 2.39-2.33 (m, 4H), 2.04 (br s, 1H), 1.69 (s, 6H).

### Intermediate-1-1-04: 1,3-bis((2,2-dimethyl-1,3-dioxan-5-yl)methoxy)propan-2-ol

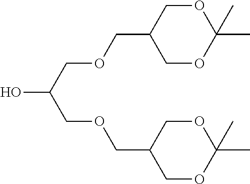 [SMILES: CC1(C)OCC(COCC(O)COCC2COC(C)(C)OC2)CO1]

To a suspension of N,N,N-tributylbutan-1-aminium iodide (CAS: 311-28-4, 0.51 g, 1.37 mmol) in (2,2-dimethyl-1,3-dioxan-5-yl)methanol (CAS: 4728-12-5, 4 g, 27.4 mmol) at rt were added KOH (1.15 g, 20.52 mmol) and H₂O (0.01 mL). The mixture was stirred at 65° C. for 1.5 h. To the mixture at 65° C. was added 2-(chloromethyl)oxirane (CAS: 106-89-8, 0.527 mL, 6.84 mmol). The mixture was stirred for 16 h at the same temperature. The mixture was cooled to 0° C. and diluted with CH₂Cl₂ and H₂O. The aqueous layer was extracted twice with CH₂Cl₂. The combined organic extracts were washed three times with brine, dried over Na₂SO₄, filtrated, and concentrated. The resulting residue was purified by silica gel flash column chromatography (gradient, hexane/EtOAc=90/10 to 0/100) to afford the title compound (0.56 g, 1.60 mmol). ¹H NMR (500 MHz, CDCl₃) δ 4.03-3.92 (m, 5H), 3.79-3.70 (m, 4H), 3.61-3.43 (m, 8H), 2.42 (d, J=4.2 Hz, 1H), 2.02-1.91 (m, 2H), 1.44 (s, 6H), 1.40 (s, 6H).

### Intermediate-1-1-05: 2,5,9,12-tetraoxatridecan-7-ol

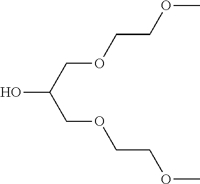

To a suspension of N,N,N-tributylbutan-1-aminium iodide (1.46 g, 3.94 mmol) in 2-methoxyethan-1-ol (CAS: 109-86-4, 6.0 g, 79 mmol) at rt were added KOH (1.151 g, 20.52 mmol) and H₂O (0.01 mL). The mixture was stirred at 65° C. for 1 h. Then the mixture was cooled to 0° C. To the mixture at 0° C. was added 2-(chloromethyl)oxirane (1.52 mL, 19.71 mmol). The mixture was stirred for 3 h at rt. Then the mixture was stirred for 4 h at 50° C. The mixture was cooled to 0° C. and diluted with CH₂Cl₂ and H₂O. The aqueous layer was extracted twice with CH₂Cl₂. The combined organic layer was washed four times with brine, dried over Na₂SO₄, filtered and concentrated. The resulting residue was purified by silica gel flash column chromatography (gradient, EtOAc/MeOH=100/0 to 90/10) to afford the title compound (2.76 g, 13.23 mmol). ¹H NMR (500 MHz, CDCl₃) δ 4.06-3.97 (m, 1H), 3.71-3.62 (m, 4H), 3.61-3.47 (m, 8H), 3.39 (s, 6H), 2.95 (s, 1H).

### Intermediate-1-2-01: (5R)-5-({[(9H-fluoren-9-yl)methoxy]carbonyl}amino)-6-oxo-6-(prop-2-en-1-yloxy)hexanoic acid

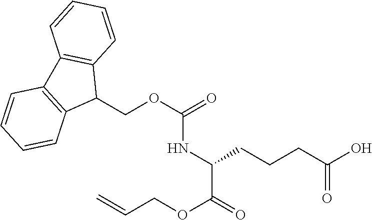 [SMILES: C=CCOC(=O)[C@@H](CCCC(=O)O)NC(=O)OCC1c2ccccc2-c2ccccc21]

To a solution of (2S)-3,6-dimethoxy-2-(propan-2-yl)-2,5-dihydropyrazine (CAS: 78342-42-4, 5.00 g, 27.14 mmol) in THF (60 mL) at −78° C. was added n-BuLi (14.00 mL, 2.5 M in hexane) dropwise. The mixture was stirred at −78° C. for 1 h. To the reaction mixture at −78° C. was added a solution of tert-butyl 4-bromobutanoate (6.65 g, 29.81 mmol) in THF (20 mL) dropwise. The resulting mixture was stirred at −78° C. for an additional 3 h. The reaction was then quenched with satd. aq. NH₄Cl (20 mL). The mixture was extracted with EtOAc. The organic extracts were washed with brine, dried over Na₂SO₄, filtered, and concentrated. The resulting residue was purified by silica gel flash column chromatography (petroleum ether/EtOAc=95/5) to afford tert-butyl 4-[(2R,5S)-3,6-dimethoxy-5-(propan-2-yl)-2,5-dihydropyrazin-2-yl]butanoate (4.50 g, 13.79 mmol). To a solution of tert-butyl 4-[(2R,5S)-3,6-dimethoxy-5-(propan-2-yl)-2,5-dihydropyrazin-2-yl]butanoate (4.50 g, 13.79 mmol), CH₃CN (54 mL) at 0° C. was added a solution of hydrogen chloride (4.5 mL) in H₂O (22.5 mL) dropwise. The mixture was stirred for 2 h at rt. The pH of the solution was adjusted to 8 with satd. aq. NaHCO₃. The mixture was extracted twice with EtOAc. The combined organic extracts were washed with brine, dried over Na₂SO₄, filtered, and concentrated. To a solution of the resulting residue in 1,4-dioxane (45 mL) and H₂O (15 mL) at 0° C. was added NaHCO₃ (2.06 g, 24.5 mmol), followed by a solution of 2, 5-dioxopyrrolidin-1-yl 9H-fluoren-9-ylmethyl carbonate (CAS: 82911-69-1, 4.56 g, 13.52 mmol) in 1,4-dioxane (15 mL) dropwise. The mixture was stirred for 2 h at rt, and then extracted with EtOAc. The organic extracts were washed with brine, dried over Na₂SO₄, filtered, and concentrated. The resulting residue was purified by silica gel flash column chromatography (petroleum ether/EtOAc=89/11). Appropriate fractions were collected and concentrated. To a solution of the residue in ClCH₂CH₂Cl (60 mL) was added trimethyltin hydroxide (4.98 g, 27.54 mmol). The mixture was stirred for 3 h at 60° C. The reaction was then quenched with satd. aq. citric acid. The mixture was extracted with EtOAc. The organic extracts were washed with brine, dried over Na₂SO₄, filtered, and concentrated. The resulting residue was purified by RP-HPLC (gradient, CH₃CN/H₂O=50/50 to 80/20) to afford (2R)-6-(tert-butoxy)-2-({[(9H-fluoren-9-yl)methoxy]carbonyl}amino)-6-oxohexanoic acid (2.21 g, 5.03 mmol). To a solution of (2R)-6-(tert-butoxy)-2-({[(9H-fluoren-9-yl)methoxy]carbonyl}amino)-6-oxohexanoic acid (1.5 g, 3.41 mmol) in DMF (8.5 mL) at rt were added DIPEA (0.89 mL, 5.12 mmol) and allyl bromide (0.38 mL, 4.4 mmol). The mixture was stirred at rt for ca. 16 h, and then diluted with H₂O. The mixture was extracted with EtOAc. The organic extracts were washed subsequently with 1M aq. HCl and brine, dried over Na₂SO₄, filtered and concentrated. The resulting residue was purified by silica gel flash column chromatography (gradient, hexane/EtOAc=100/0 to 50/50). Appropriate fractions were concentrated. A mixture of the resulting residue (1.37 g, 2.86 mmol) and TFA (0.43 mL) in CH₂Cl₂ (7 mL) was stirred for ca. 16 h at rt. The mixture was concentrated to furnish the title compound (1.19 g, 2.81 mmol) which was used in the next step without any further purification. MS (ESI+): m/z 424.3 [M+H].

### Intermediate-1-3-01: (2S)-2-({[(9H-fluoren-9-yl)methoxy]carbonyl}amino)-6-{[(4-nitrophenoxy)carbonyl]amino}hexanoic acid

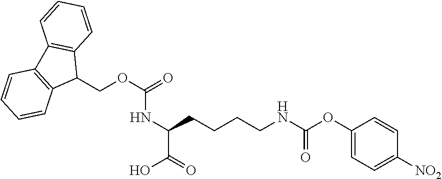 [SMILES: O=C(N[C@@H](CCCCNC(=O)Oc1ccc([N+](=O)[O-])cc1)C(=O)O)OCC1c2ccccc2-c2ccccc21]

To a suspension of (2S)-6-amino-2-({[(9H-fluoren-9-yl)methoxy]carbonyl}amino)hexanoic acid hydrochloride (1 g, 2.47 mmol) in THF (12.4 mL) at rt was added MSA (CAS: 7449-74-3, 0.79 g, 5.43 mmol). The mixture was stirred at rt for 0.5 h. To the solution at 0° C. were added DIPEA (0.52 mL, 2.96 mmol) and 4-nitrophenyl chloroformate (CAS: 7693-46-1, 0.597 g, 2.96 mmol). The mixture was stirred for 2 h at 0° C. The reaction was quenched with 2M aq. HCl. The mixture was extracted twice with EtOAc. The combined organic extracts were washed successively with H₂O twice, brine, dried over Na₂SO₄, filtered, and concentrated. The resulting residue was purified by silica gel flash chromatography (gradient, CH₂Cl₂/MeOH=98/2 to 60/40) to afford the title compound (0.77 g, 1.41 mmol). MS (ESI+): m/z 534.3 [M+H].

### Intermediate-1-3-02: (2S)-2-({[(9H-fluoren-9-yl)methoxy]carbonyl}amino)-7-{[(4-nitrophenoxy)carbonyl]amino}heptanoic acid

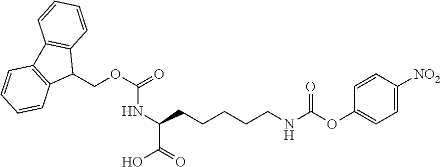 [SMILES: O=C(N[C@@H](CCCCCNC(=O)Oc1ccc([N+](=O)[O-])cc1)C(=O)O)OCC1c2ccccc2-c2ccccc21]

To a solution of (2S)-7-{[(tert-butoxy)carbonyl]amino}-2-({[(9H-fluoren-9-yl)methoxy]carbonyl}amino)heptanoic acid (10 g, 20.72 mmol) in CH₂Cl₂ (60 mL) were added 4 M HCl in CPME (60 mL). The mixture was stirred at rt for 4 h. The resulting solid was collected by filtration and washed by iPr₂O to afford (S)-2-((((9H-fluoren-9-yl)methoxy)carbonyl)amino)-7-aminoheptanoic acid hydrochloride (8.5 g, 20.29 mmol). To a suspension of the obtained compound (5 g, 11.94 mmol) in THF (60 mL) at rt was added MSA (3.81 g, 26.2 mmol). The mixture was stirred for 0.5 h at the same temperature, which turned into a clear solution. To the reaction mixture at 0° C. were added DIPEA (2.502 mL, 14.32 mmol) and 4-nitrophenyl chloroformate (2.89 g, 14.32 mmol). The mixture was stirred for 2 h at the same temperature and then quenched with 1 M aq. HCl. The mixture was extracted twice with EtOAc. The combined organic extracts were washed with H₂O, brine, dried over Na₂SO₄, filtered and concentrated. The resulting residue was suspended with MTBE. The resulted solid was collected by filtration to afford the title compound (5.5 g, 10.04 mmol). MS (ESI+): m/z 548.4 [M+H].

### Intermediate-1-3-03: (2S)-2-({[(9H-fluoren-9-yl)methoxy]carbonyl}amino)-3-[4-(2-{[(4-nitrophenoxy)carbonyl]amino}ethoxy)phenyl]propanoic acid

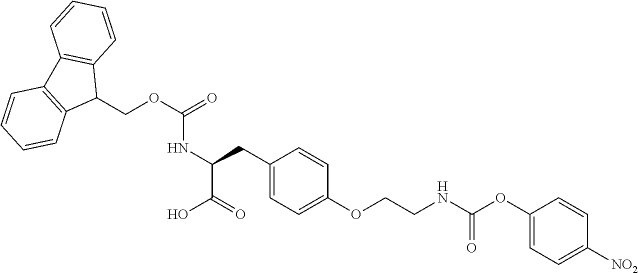 [SMILES: O=C(N[C@@H](Cc1ccc(OCCNC(=O)Oc2ccc([N+](=O)[O-])cc2)cc1)C(=O)O)OCC1c2ccccc2-c2ccccc21]

To a solution of (2S)-3-[4-(2-{[(tert-butoxy)carbonyl]amino}ethoxy)phenyl]-2-({[(9H-fluoren-9-yl)methoxy]carbonyl}amino)propanoic acid (CAS: 1013883-02-7, 5.47 g, 10.00 mmol) in CH₂Cl₂ (33 mL) were added 4M HCl in MTHP (25 mL). The mixture was stirred at rt for 5 h. The resulting solid was collected by filtration and washed by iPr₂O. To a suspension of the obtained solid (as hydrochloride, 4.59 g, 9.50 mmol) in THF (25 mL) at rt was added MSA (4.83 g, 23.76 mmol). The mixture was stirred for 0.5 h at the same temperature, which turned into a clear solution. To the mixture at 0° C. were added DIPEA (1.992 mL, 11.40 mmol) and 4-nitro phenyl chloroformate (1.877 g, 9.31 mmol). The mixture was stirred for 1 h at the same temperature and then quenched with 2M aq. HCl. The mixture was extracted twice with EtOAc. The combined organic extracts were washed with H₂O, brine, dried over Na₂SO₄, filtered and concentrated. The resulting residue was suspended with iPr₂O/heptane (50 mL/50 mL). The resulted solid was collected by filtration to afford the title compound (5.921 g, 9.00 mmol). MS (ESI+): m/z 612.0 [M+H].

### Example-1-1-01: (2S)-2-[[(9H-fluoren-9-ylmethoxy)carbonyl]amino]-6-(4-methylpiperazine-1-carbonylamino)hexanoic acid hydrochloride

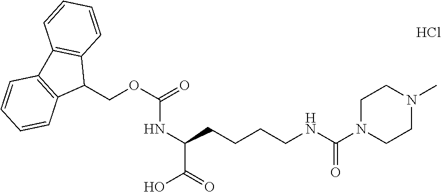 [SMILES: CN1CCN(C(=O)NCCCC[C@H](NC(=O)OCC2c3ccccc3-c3ccccc32)C(=O)O)CC1 ; Cl]

To a mixture of prop-2-en-1-yl (2S)-6-amino-2-({[(9H-fluoren-9-yl)methoxy]carbonyl}amino)hexanoate hydrochloride (CAS: 815619-80-8, 10.0 g, 22.47 mmol) in CH₂Cl₂ (50 mL) and satd. aq. NaHCO₃ (50 mL) at 0° C. was added triphosgene (CAS: 503-38-8, 2.67 g, 8.99 mmol) portionwise. The mixture was stirred for 0.5 h at the same temperature. To the mixture at 0° C. was added 1-methylpiperazine (2.25 g, 22.47 mmol) portionwise. The mixture was stirred at rt for 2 h, and then diluted with CH₂Cl₂.

The mixture was washed successively with H₂O and brine, dried over Na₂SO₄, filtered, and then concentrated. The resulting residue was purified by silica gel flash column chromatography (gradient, CH₂Cl₂/MeOH=100/0 to 90/10) to afford 8.0 g of the desired product. To a solution of the product (5.00 g, 9.35 mmol) in CH₂Cl₂ (50 mL) under N₂ atmosphere were added phenylsilane (2.02 g, 18.70 mmol) and Pd(PPh₃)₄ (0.54 g, 0.47 mmol). The mixture was stirred at room temperature, and then diluted with CH₂Cl₂ (ca. 50 mL). The mixture was washed successively three times with H₂O and twice with brine, and then dried over Na₂SO₄, filtered, and then concentrated. The resulting residue was purified by silica gel flash column chromatography (gradient, CH₂Cl₂/MeOH=100/0 to 88/12). Appropriate fractions were collected and concentrated.

The resulting residue was dissolved in EtOAc (20 mL), and subsequently added HCl in EtOAc (2M, 20 mL). The mixture was stirred at rt for 3 h. Resulting solid material was collected by filtration to furnish the title compound (3.18 g, 5.99 mmol). ¹H NMR (500 MHz, DMSO-d₆) δ 12.56 (br s, 1H), 10.49 (br s, 1H), 8.06 (d, J=7.63 Hz, 1H), 7.90 (d, J=7.5 Hz, 2H), 7.73 (dd, J=2.6, 7.3 Hz, 2H), 7.64 (d, J=7.9 Hz, 1H), 7.42 (dd, J=7.5, 7.5 Hz, 2H), 7.33 (dd, J=7.5, 7.5 Hz, 2H), 6.76 (br t, J=5.2 Hz, 1H), 5.20-5.11 (m, 1H), 4.32-4.21 (m, 3H), 4.15-3.94 (m, 2H), 3.94-3.84 (m, 1H), 3.18-2.97 (m, 4H), 2.96-2.85 (m, 2H), 2.74 (s, 3H), 1.74-1.54 (m, 2H), 1.46-1.26 (m, 4H). MS (ESI+): m/z 495.4 [M+H].

### Example-1-1-02: (2S)-6-({4-[2-(tert-butoxy)-2-oxoethyl]piperazine-1-carbonyl}amino)-2-({[(9H-fluoren-9-yl)methoxy]carbonyl}amino)hexanoic acid

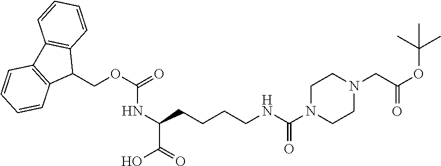 [SMILES: CC(C)(C)OC(=O)CN1CCN(C(=O)NCCCC[C@H](NC(=O)OCC2c3ccccc3-c3ccccc32)C(=O)O)CC1]

To a solution of prop-2-en-1-yl (2S)-6-amino-2-({[(9H-fluoren-9-yl)methoxy]carbonyl}amino)hexanoate hydrochloride (CAS: 815619-80-8, 3.34 g, 7.5 mmol) and DIPEA (4.18 mL, 24.00 mmol) in DCM (30 mL) at 0° C. was added triphosgene (1.00 g, 3.38 mmol The mixture was stirred at 0° C. for 0.25 h, and then 25° C. for 0.75 h. To the mixture at 0° C. were added tert-butyl 2-(piperazin-1-yl)acetate (CAS: 112257-22-4, 1.65 g, 8.25 mmol) and 1M aq. NaHCO₃ (50 mL). The mixture was then stirred at 0° C. for 1 h, and then extracted with EtOAc. The organic extract was washed successively with 5% aq. AcOH, H₂O, and satd. aq. NaHCO₃, dried over Na₂SO₄, filtered and concentrated. The resulting residue was purified by silica gel flash column chromatography (gradient, hexane/EtOAc=100/0 to 0/100). Appropriate fractions were concentrated. To a solution of the resulting residue (2.13 g, 3.36 mmol) in DCM (30 mL) were added phenylsilane (0.48 mL, 3.90 mmol) and Pd(PPh₃)₄ (0.04 g, 0.04 mmol) at 0° C. The mixture was then stirred at 25° C. for 15 h, and then concentrated. The resulting residue was purified by silica gel flash column chromatography (gradient, hexane/EtOAc=50/50 to 0/100, then DCM/MeOH=100/0 to 85/15) to afford the title compound (1.51 g, 2.54 mmol). ¹H NMR (500 MHz, DMSO-d₆) δ 12.52 (br s, 1H), 7.90 (d, J=7.6 Hz, 2H), 7.73 (d, J=7.5 Hz, 2H), 7.68-7.55 (m, 1H), 7.42 (dd, J=7.5, 7.5 Hz, 2H), 7.33 (dd, J=7.1, 7.1 Hz, 2H), 6.45 (br t, J=5.3 Hz, 1H), 4.32-4.19 (m, 3H), 3.93-3.85 (m, 1H), 3.30-3.19 (m, 4H), 3.09 (s, 2H), 3.03-2.95 (m, 2H), 2.46-2.32 (m, 4H), 1.72-1.55 (m, 2H), 1.39 (s, 9H), 1.38-1.23 (m, 4H). MS (ESI+): m/z 595.5 (M+H)

### Example-1-1-03: (2S)-2-({[(9H-fluoren-9-yl)methoxy]carbonyl}amino)-6-({methyl[(2S,3R,4R,5R)-2,3,4,5-tetrahydroxy-6-{[tris(propan-2-yl)silyl]oxy}hexyl]carbamoyl}amino)hexanoic acid

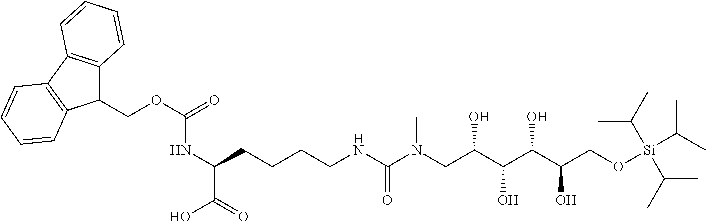 [SMILES: CC(C)[Si](OC[C@@H](O)[C@@H](O)[C@H](O)[C@@H](O)CN(C)C(=O)NCCCC[C@H](NC(=O)OCC1c2ccccc2-c2ccccc21)C(=O)O)(C(C)C)C(C)C]

To a solution of prop-2-en-1-yl (2S)-6-amino-2-({[(9H-fluoren-9-yl)methoxy]carbonyl}amino)hexanoate hydrochloride (8 g, 17.98 mmol) in CH₂Cl₂ (80 mL) were added bis(trichloromethyl) carbonate (2.67 g, 8.99 mmol) at 0° C. Then to the mixture was added DIPEA (10.05 mL, 57.5 mmol) dropwise and stirred for 0.5 h at 0° C. To the reaction mixture at 0° C. was added 1,4-dioxane (80 mL), followed by (2R,3R,4R,5S)-6-(methylamino)hexane-1,2,3,4,5-pentaol (CAS: 6284-40-8, 14.04 g, 71.9 mmol) in 0.5 M aq. NaHCO₃80 mL). The mixture was stirred at 0° C. for 1.5 h and then extracted with EtOAc. The organic layer was washed with H₂O, 1M aq. HCl, satd. aq. NaHCO₃, brine, dried over Na₂SO₄, filtered and concentrated. The resulting residue was purified by silica gel flash column chromatography (gradient, hexane/EtOAc=100/0 to 0/100, then CH₂Cl₂/MeOH=100/0 to 60/40) to afford prop-2-en-1-yl (2S)-2-({[(9H-fluoren-9-yl)methoxy]carbonyl}amino)-6-({methyl[(2S,3R,4R,5R)-2,3,4,5,6-pentahydroxyhexyl]carbamoyl}amino)hexanoate (8.7 g, 13.82 mmol). To a solution of prop-2-en-1-yl (2S)-2-({[(9H-fluoren-9-yl)methoxy]carbonyl}amino)-6-({methyl[(2S,3R,4R,5R)-2,3,4,5,6-pentahydroxyhexyl]carbamoyl}amino)hexanoate (3.01 g, 4.78 mmol) in CH₂Cl₂ (34.1 mL) were added imidazole (0.81 g, 11.95 mmol) and chlorotriisopropylsilane (1.27 mL, 5.98 mmol). The mixture was stirred for ca. 5 days, and then diluted with EtOAc. The organic layer was washed three times with H₂O, dried over Na₂SO₄, filtered and concentrated. The resulting residue was purified by silica gel flash column chromatography (gradient, hexane/EtOAc=100/0 to 40/60, then CH₂Cl₂/MeOH=100/0 to 90/10) to afford prop-2-en-1-yl (2S)-2-({[(9H-fluoren-9-yl)methoxy]carbonyl}amino)-6-({methyl[(2S,3R,4R,5R)-2,3,4,5-tetrahydroxy-6-{[tris(propan-2-yl)silyl]oxy}hexyl]carbamoyl}amino)hexanoate (3.16 g, 4.02 mmol).

To a solution of prop-2-en-1-yl (2S)-2-({[(9H-fluoren-9-yl)methoxy]carbonyl}amino)-6-({methyl[(2S,3R,4R,5R)-2,3,4,5-tetrahydroxy-6-{[tris(propan-2-yl)silyl]oxy}hexyl]carbamoyl}amino)hexanoate (3.16 g, 4.02 mmol) in CH₂Cl₂ (20 mL) at rt were added phenylsilane (0.99 mL, 8.04 mmol) and Pd(PPh₃)₄ (0.12 g, 0.10 mmol). The mixture was stirred for 2 h at rt, and then concentrated. The resulting residue was purified by silica gel flash column chromatography (gradient, hexane/EtOAc=100/0 to 40/60, then CH₂Cl₂/MeOH=100/0 to 70/30) to afford the title compound (2.84 g, 3.24 mmol). ¹H NMR (300 MHz, DMSO-d₆) δ 7.89 (d, J=7.5 Hz, 2H), 7.83-7.54 (m, 2H), 7.52-7.19 (m, 4H), 6.85 (s, 1H), 6.22 (s, 1H), 4.62 (d, J=5.7 Hz, 3H), 4.35-4.14 (m, 3H), 3.97-3.54 (m, 6H), 3.54-3.44 (m, 2H), 3.15-2.88 (m, 3H), 2.80 (s, 3H), 1.78-1.16 (m, 6H), 1.16-0.52 (m, 19H). MS (ESI+): m/z 746.6 [M+H].

### Example-1-1-04: (2R)-6-({[(3R)-1-[2-(tert-butoxy)-2-oxoethyl]pyrrolidin-3-yl]carbamoyl}amino)-2-({[(9H-fluoren-9-yl)methoxy]carbonyl}amino)hexanoic acid

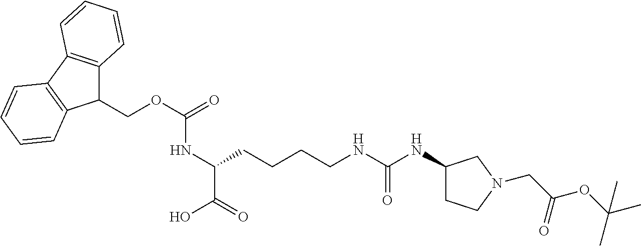 [SMILES: CC(C)(C)OC(=O)CN1CC[C@@H](NC(=O)NCCCC[C@@H](NC(=O)OCC2c3ccccc3-c3ccccc32)C(=O)O)C1]

To a suspension of prop-2-en-1-yl (2R)-6-amino-2-({[(9H-fluoren-9-yl)methoxy]carbonyl}amino)hexanoate hydrochloride (CAS: 1272754-92-3, 3 g, 6.74 mmol) in CH₂Cl₂ (27.0 mL) at 0° C. was added triphosgene (0.90 g, 3.03 mmol), followed by DIPEA (3.77 mL, 21.58 mmol) at 0° C. dropwise over 2 min. The mixture was stirred at 0° C. for 0.25 h. To the reaction mixture at 0° C. were added sodium bicarbonate (2.27 g, 27.0 mmol) and tert-butyl (R)-3-aminopyrrolidine-1-carboxylate (3.14 g, 16.86 mmol). The mixture was stirred for ca. 0.25 h at rt, and then extracted twice with CH₂Cl₂. The combined organic extracts were washed three times with 1N HCl, H₂O, satd. aq. NaHCO₃, dried over Na₂SO₄, filtered and concentrated. The resulting residue was purified by silica gel flash column chromatography (gradient, hexane/EtOAc=6/4 to 0/1).

Appropriate fractions were concentrated. To a solution of the resulting residue (3.05 g, 4.91 mmol) in CH₂Cl₂ (19 mL) at 0° C. was added 4M HCl in CPME (3.6 mL, 14.50 mmol). The mixture was stirred for ca. 16 h at rt and then concentrated. The resulting residue was suspended in toluene and then concentrated. To the resulting residue in CH₂Cl₂ (19 mL) at 0° C. were added DIPEA (2.11 mL, 12.08 mmol) and tert-butyl bromoacetate (0.786 mL, 5.32 mmol) dropwise. The mixture was stirred for 3 h at 0° C. and then diluted with CH₂Cl₂. The organic phase was washed successively with satd. aq. NaHCO₃, brine, dried over Na₂SO₄, filtered, and concentrated. The resulting residue was purified by silica gel flash column chromatography (gradient, CH₂Cl₂/MeOH=100/0 to 90/10) to afford prop-2-en-1-yl (2R)-6-({[(3R)-1-[2-(tert-butoxy)-2-oxoethyl]pyrrolidin-3-yl]carbamoyl}amino)-2-({[(9H-fluoren-9-yl)methoxy]carbonyl}amino)hexanoate (1.70 g, 2.68 mmol).

To a solution of prop-2-en-1-yl (2R)-6-({[(3R)-1-[2-(tert-butoxy)-2-oxoethyl]pyrrolidin-3-yl]carbamoyl}amino)-2-({[(9H-fluoren-9-yl)methoxy]carbonyl}amino)hexanoate (1.70 g, 2.68 mmol) in CH₂Cl₂ (11 mL) at 0° C. were added phenylsilane (0.66 mL, 5.36 mmol) and Pd(PPh₃)₄ (0.062 g, 0.054 mmol). The mixture was stirred for 1 h at rt, and then concentrated. The residue was purified by silica gel flash column chromatography (gradient, CH₂Cl₂/MeOH=100/0 to 60/40) to afford the title compound (1.51 g, 2.52 mmol). ¹H NMR (500 MHz, DMSO-d₆) δ 12.60 (br s, 1H), 7.90 (d, J=7.5 Hz, 2H), 7.72 (d, J=7.2 Hz, 2H), 7.42 (dd, J=7.3, 7.3 Hz, 2H), 7.33 (dd, J=7.4, 7.4 Hz, 2H), 5.93 (d, J=7.5 Hz, 1H), 5.80-5.75 (m, 1H), 4.32-4.20 (m, 3H), 4.05-3.99 (m, 1H), 3.91-3.75 (m, 1H), 3.15 (s, 2H), 3.00-2.90 (m, 2H), 2.75-2.63 (m, 2H), 2.48-2.32 (m, 3H), 1.69-1.67 (m, 1H), 1.62-1.52 (m, 1H), 1.43-1.41 (m, 2H), 1.40 (s, 9H), 1.36-1.27 (m, 3H). MS (ESI+): m/z 595.6 [M+H].

### Example-1-1

The following compounds were synthesized as outlined for the preparation of Example-1-1-01, Example-1-1-02, Example-1-1-03, or Example-1-1-04 employing appropriate starting materials in the table.

| | Structure and IUPAC name |
|---|---|
| | Fmoc-protected starting material-A |
| | Starting material-B |
| Example | NMR and MS |
| 1-1-05 | 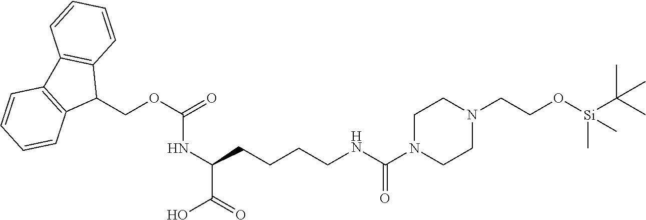 [SMILES: CC(C)(C)[Si](C)(C)OCCN1CCN(C(=O)NCCCC[C@H](NC(=O)OCC2c3ccccc3-c3ccccc32)C(=O)O)CC1] |
| | (2S)-6-[(4-{2-[(tert-butyldimethylsilyl)oxy]ethyl}piperazine-1-carbonyl) |
| | amino]-2-({[(9H-fluoren-9-yl)methoxy]carbonyl}amino)hexanoic acid |
| | prop-2-en-1-yl (2S)-6-amino-2-({[(9H-fluoren-9-yl)methoxy]carbonyl}amino) |
| | hexanoate hydrochloride |
| | 1-[2-[(tert-butyldimethylsilyl)oxy]ethyl]piperazine (CAS: 676341-98-3) |
| | ¹H NMR (500 MHz, DMSO-d₆) δ 12.57 (br s, 1H), 7.89 (d, J = 7.5 Hz, |
| | 2H), 7.72 (d, J = 7.5 Hz, 2H), 7.65-7.51 (m, 1H), 7.42 (t, J = |
| | 7.5 Hz, 2H), 7.33 (t, J = 7.5 Hz, 2H), 6.44 (d, J = 5.5 Hz, 1H), 4.34-4.25 |
| | (m, 2H), 4.24-4.15 (m, 1 H), 3.89-3.85 (m, 1H), 3.64 (t, J = 6.0 Hz, |
| | 2H), 3.21-3.19 (m, 4H), 2.97-2.93 (m, 2H), 2.37 (t, J = 6.0 Hz, |
| | 2H) 2.33-2.31 (m, 4H), 1.70-1.54 (m, 2H), 1.45-1.25 (m, 4H), 0.85 |
| | (s, 9H), 0.02 (s, 6H). MS (ESI+): m/z 639.5 [M + H]. |
| 1-1-06 | 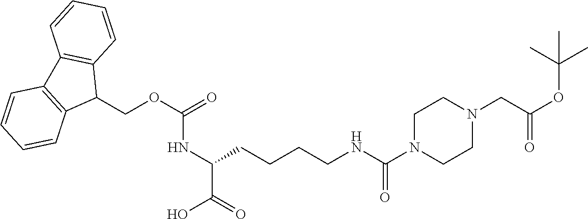 [SMILES: CC(C)(C)OC(=O)CN1CCN(C(=O)NCCCC[C@@H](NC(=O)OCC2c3ccccc3-c3ccccc32)C(=O)O)CC1] |
| | (2R)-6-({4-[2-(tert-butoxy)-2-oxoethyl]piperazine-1-carbonyl}amino)-2- |
| | ({[(9H-fluoren-9-yl)methoxy]carbonyl}amino)hexanoic acid |
| | prop-2-en-1-yl (2R)-6-amino-2-({[(9H-fluoren-9-yl)methoxy]carbonyl} |
| | amino)hexanoate hydrochloride |
| | tert-butyl 2-(piperazin-1-yl)acetate |
| | Substantially identical to Example-1-1-02 |
| 1-1-07 | 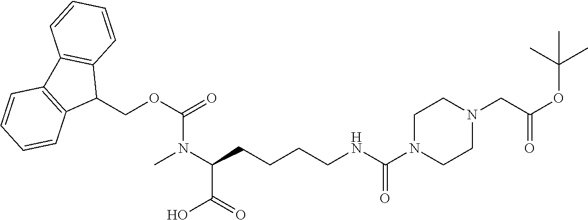 [SMILES: CN(C(=O)OCC1c2ccccc2-c2ccccc21)[C@@H](CCCCNC(=O)N1CCN(CC(=O)OC(C)(C)C)CC1)C(=O)O] |
| | (2S)-6-({4-[2-(tert-butoxy)-2-oxoethyl]piperazine-1-carbonyl}amino)-2- |
| | ({[(9H-fluoren-9-yl)methoxy]carbonyl}(methyl)amino)hexanoic acid |
| | Reference example-1-1-01 |
| | tert-butyl piperazine-1-carboxylate |
| | ¹H NMR (500 MHz, DMSO-d₆) δ 12.82 (br s, 1H), 7.98-7.81 (m, 2H), |
| | 7.73-7.53 (m, 2H), 7.45-7.38 (m, 2H), 7.36-7.28 (m, 2H), 6.44 |
| | (br, 1H), 4.50-4.23 (m, 4H), 3.27-3.21 (m, 4H), 3.09 (s, 2H), 3.05- |
| | 2.94 (m, 2H), 2.78-2.68 (m, 3H), 2.48-2.36 (m, 4H), 1.83-1.57 |
| | (m, 2H), 1.39 (s, 9H), 1.38-1.23 (m, 2H), 1.21-1.03 (m, 2H). MS |
| | (ESI+): m/z 609.4 |
| 1-1-08 | 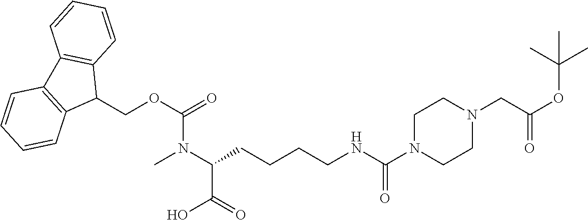 [SMILES: CN(C(=O)OCC1c2ccccc2-c2ccccc21)[C@H](CCCCNC(=O)N1CCN(CC(=O)OC(C)(C)C)CC1)C(=O)O] |
| | (2R)-6-({4-[2-(tert-butoxy)-2-oxoethyl]piperazine-1-carbonyl}amino)-2- |
| | ({[(9H-fluoren-9-yl)methoxy]carbonyl}(methyl)amino)hexanoic acid |
| | Reference example-1-1-02 |
| | tert-butyl piperazine-1-carboxylate |
| | Substantially identical to Example-1-1-07. |
| 1-1-09 | 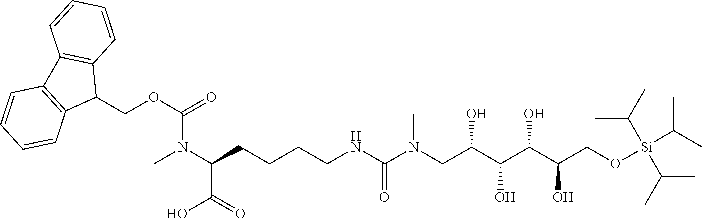 [SMILES: CC(C)[Si](OC[C@@H](O)[C@@H](O)[C@H](O)[C@@H](O)CN(C)C(=O)NCCCC[C@@H](C(=O)O)N(C)C(=O)OCC1c2ccccc2-c2ccccc21)(C(C)C)C(C)C] |
| | (2S)-2-({[(9H-fluoren-9-yl)methoxy]carbonyl}(methyl)amino)-6-({methyl |
| | [(2S,3R,4R,5R)-2,3,4,5-tetrahydroxy-6-{[tris(propan-2-yl)silyl]oxy}hexyl] |
| | carbamoyl}amino)hexanoic acid |
| | Reference example-1-1-01 |
| | (2R,3R,4R,5S)-6-(methylamino)hexane-1,2,3,4,5-pentaol |
| | ¹H NMR (500 MHz, DMSO-d₆) δ 7.92-7.87 (m, 2 H), 7.67-7.60 (m, |
| | 2 H), 7.45-7.38 (m, 2 H), 7.36-7.29 (m, 2 H), 6.24 (t, J = 5.5 Hz, |
| | 1 H), 5.05 (br s, 1 H), 4.46-4.23 (m, 6 H), 3.84 (dd, J = 10.0, 2.5 Hz, |
| | 1 H), 3.70 (br s, 1 H), 3.62 (dd, J = 10.0, 6.0 Hz, 1 H), 3.55 (brs, 2 H), |
| | 3.49 (d, J = 8.0 Hz, 1 H), 3.34-3.29 (m, 1 H), 3.17-3.12 (dd, J = |
| | 14.5, 7.5 Hz, 1 H), 3.00-2.98 (m, 2 H), 2.80 (s, 3 H), 2.74-2.72 |
| | (m, 3 H), 1.83-1.76 (m, 1 H), 1.70-1.54 (m, 1 H), 1.43-1.37 (m, |
| | 2 H), 1.18-1.02 (m, 24 H). MS(ESI+): m/z 760.6 [M + H]. |
| 1-1-10 | 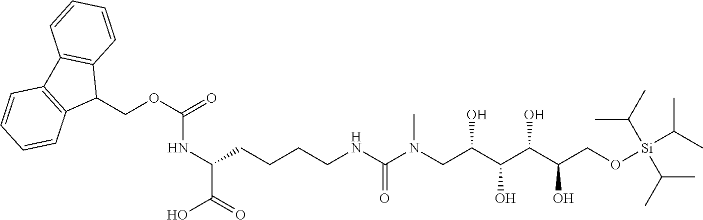 [SMILES: CC(C)[Si](OC[C@@H](O)[C@@H](O)[C@H](O)[C@@H](O)CN(C)C(=O)NCCCC[C@@H](NC(=O)OCC1c2ccccc2-c2ccccc21)C(=O)O)(C(C)C)C(C)C] |
| | (2R)-2-({[(9H-fluoren-9-yl)methoxy]carbonyl}amino)-6-({methyl[(2S,3R, |
| | 4R,5R)-2,3,4,5-tetrahydroxy-6-{[tris(propan-2-yl)silyl]oxy}hexyl]carbamoyl} |
| | amino)hexanoic acid |
| | prop-2-en-1-yl (2R)-6-amino-2-({[(9H-fluoren-9-yl)methoxy]carbonyl} |
| | amino)hexanoate hydrochloride |
| | (2R,3R,4R,5S)-6-(methylamino)hexane-1,2,3,4,5-pentaol |
| | ¹H NMR (500 MHz, DMSO-d₆) δ 7.89 (d, J = 7.5 Hz, 2 H), 7.71 (d, |
| | J = 7.5 Hz, 2 H), 7.42 (dd, J = 7.5, 7.5 Hz, 2 H), 7.33 (dd, J = 7.5, 7.5 Hz, |
| | 2 H), 6.22 (s, 1 H), 5.20 (br s, 1 H), 4.65 (br s, 2 H), 4.30-4.20 |
| | (m, 3 H), 3.86-3.82 (m, 2 H), 3.72-3.71 (m, 1 H), 3.62 (dd, J = 10.0, |
| | 6.0 Hz, 1 H), 3.56 (br s, 2 H), 3.48 (d, J = 7.5 Hz, 1 H), 3.33-3.30 |
| | (m, 1 H), 3.17-3.10 (m, 1 H), 3.00-2.98 (m, 2 H), 2.80 (s, 3 H), |
| | 1.69-1.57 (m, 2 H), 1.41-1.23 (m, 4 H), 1.10-1.03 (m, 21 H); MS |
| | (ESI+): m/z 746.6 [M + H]. |
| 1-1-11 | 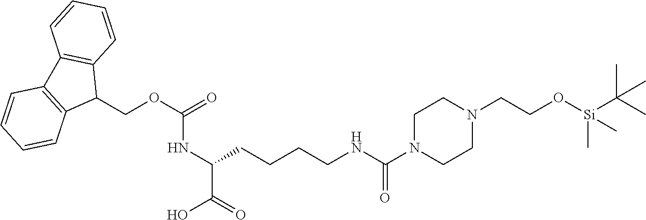 [SMILES: CC(C)(C)[Si](C)(C)OCCN1CCN(C(=O)NCCCC[C@@H](NC(=O)OCC2c3ccccc3-c3ccccc32)C(=O)O)CC1] |
| | (2R)-6-[(4-{2-[(tert-butyldimethylsilyl)oxy]ethyl}piperazine-1-carbonyl) |
| | amino]-2-({[(9H-fluoren-9-yl)methoxy]carbonyl}amino)hexanoic acid |
| | prop-2-en-1-yl (2R)-6-amino-2-({[(9H-fluoren-9-yl)methoxy]carbonyl} |
| | amino)hexanoate hydrochloride |
| | 1-[2-[(tert-butyldimethylsilyl)oxy]ethyl]piperazine (CAS: 676341-98-3) |
| | Substantially identical to Example-1-1-05 |
| 1-1-12 | 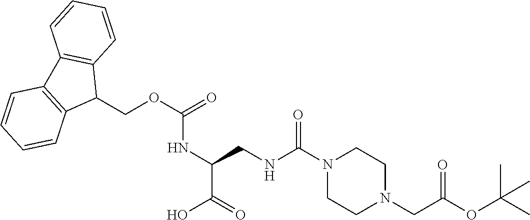 [SMILES: CC(C)(C)OC(=O)CN1CCN(C(=O)NC[C@H](NC(=O)OCC2c3ccccc3-c3ccccc32)C(=O)O)CC1] |
| | (2S)-3-({4-[2-(tert-butoxy)-2-oxoethyl]piperazine-1-carbonyl}amino)-2- |
| | ({[(9H-fluoren-9-yl)methoxy]carbonyl}amino)propanoic acid |
| | Reference example-1-1-03 |
| | tert-butyl 2-(piperazin-1-yl)acetate |
| | ¹H NMR (500 MHz, CDCl₃) δ 7.76 (d, J = 7.5 Hz, 2H), 7.59 (dd, J = 6.0, |
| | 5.5 Hz, 2H), 7.39 (dd, J = 7.0, 7.0 H, 2H), 7.31 (dd, J = 7.5, 7.5 Hz, |
| | 2H), 6.55 (br s, 1H), 5.69 (brs, 1H), 4.42-4.29 (m, 2H), 4.27-4.15 |
| | (m, 2H), 3.84-3.70 (m, 1H), 3.55-3.34 (m, 5H), 3.20-3.14 (m, 2H), |
| | 2.67-2.61 (m, 4H), 1.44 (s, 9H); MS (ESI+): m/z 553.5 [M + H]. |
| 1-1-13 | 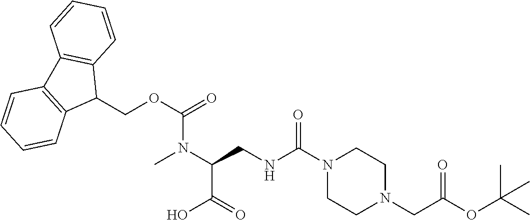 [SMILES: CN(C(=O)OCC1c2ccccc2-c2ccccc21)[C@@H](CNC(=O)N1CCN(CC(=O)OC(C)(C)C)CC1)C(=O)O] |
| | (2S)-3-({4-[2-(tert-butoxy)-2-oxoethyl]piperazine-1-carbonyl}amino)-2- |
| | ({[(9H-fluoren-9-yl)methoxy]carbonyl}(methyl)amino)propanoic acid |
| | Reference example-1-1-04 |
| | tert-butyl 2-(piperazin-1-yl)acetate |
| | ¹H NMR (500 MHz, CDCl₃) δ 7.80-7.68 (m, 2H), 7.62-7.50 (m, 2H), |
| | 7.42-7.32 (m, 2H), 7.32-7.21 (m, 2H), 5.79-5.25 (m, 1H), 5.25- |
| | 4.01 (m, 6H), 3.83-3.60 (m, 1H), 3.48-3.20 (m, 4H), 3.20-2.76 |
| | (m, 6H), 2.76-2.19 (m, 4H), 1.48-1.36 (m, 9H); MS (ESI+): m/z 567.5 |
| | [M + H]. |
| 1-1-14 | 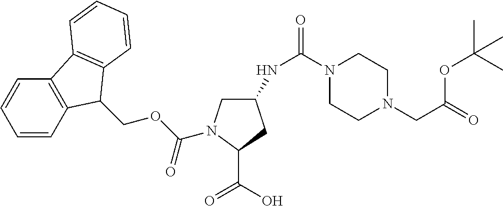 [SMILES: CC(C)(C)OC(=O)CN1CCN(C(=O)N[C@@H]2C[C@@H](C(=O)O)N(C(=O)OCC3c4ccccc4-c4ccccc43)C2)CC1] |
| | (2S,4R)-4-({4-[2-(tert-butoxy)-2-oxoethyl]piperazine-2-carbonyl}amino)-1- |
| | {[(9H-fluoren-9-yl)methoxy]carbonyl}pyrrolidine-2-carboxylic acid |
| | Reference example-1-1-05 |
| | tert-butyl 2-(piperazin-1-yl)acetate |
| | ¹H NMR (500 MHz, CDCl₃) δ 7.71 (br s, 2H), 7.63-7.47 (m, 2H), 7.43- |
| | 7.31 (m, 2H), 7.29-7.22 (m, 2H), 5.13-4.88 (m, 1H), 4.46-4.35 |
| | (br s, 3H), 4.25-4.10 (m, 2H), 3.97-3.84 (m, 1H), 3.46-3.27 (m, |
| | 5H), 3.18-3.10 (m, 2H), 2.63-2.54 (m, 4H), 2.47-2.29 (m, 1H), 2.23- |
| | 2.12 (m, 1H), 1.43 (s, 9H) MS (ESI+): m/z 579.5 [M + H]. |
| 1-1-15 | 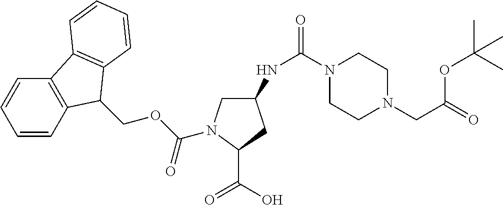 [SMILES: CC(C)(C)OC(=O)CN1CCN(C(=O)N[C@H]2C[C@@H](C(=O)O)N(C(=O)OCC3c4ccccc4-c4ccccc43)C2)CC1] |
| | (2S,4S)-4-({4-[2-(tert-butoxy)-2-oxoethyl]piperazine-1-carbonyl}amino)-1- |
| | {[(9H-fluoren-9-yl)methoxy]carbonyl}pyrrolidine-2-carboxylic acid |
| | Reference example-1-1-06 |
| | tert-butyl 2-(piperazin-1-yl)acetate |
| | ¹H NMR (500 MHz, CDCl₃) δ 7.77-7.74 (m, 2H), 7.68-7.54 (m, 2H), |
| | 7.42-7.37 (m, 2H), 7.32-7.29 (m, 2H), 6.63-6.61 (m, 1H), 4.68 (m, |
| | 1H), 4.48-4.18 (m, 5H), 3.73-3.63 (m, 4H), 3.28-3.18 (m, 4H), 2.97- |
| | 2.86 (m, 3H), 2.53-2.41 (m, 1H), 2.08 (d, J = 14.0 Hz, 1H), 1.37- |
| | 1.36 (m, 9H). MS (ESI+): m/z 579.5 [M + H]. |

### Example-1-2-01: (2S)-2-({[(9H-fluoren-9-yl)methoxy]carbonyl}amino)-6-[1(4-{2-oxo-2-[(2-phenylpropan-2-yl)oxy]ethyl}piperazine-1-carbonyl)amino]hexanoic acid

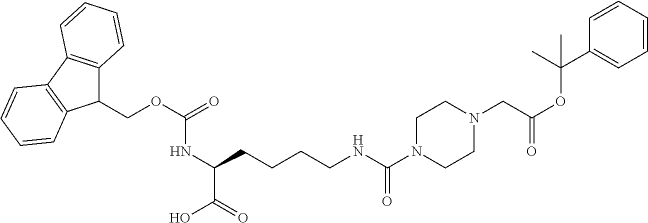 [SMILES: CC(C)(OC(=O)CN1CCN(C(=O)NCCCC[C@H](NC(=O)OCC2c3ccccc3-c3ccccc32)C(=O)O)CC1)c1ccccc1]

To a solution of Intermediate-1-3-01 (4.98 g, 9.34 mmol) in THF (28 mL) at 0° C. was added dropwise a solution of Intermediate-1-1-03 (2.57 g, 9.81 mmol) and DIPEA (1.80 mL, 10.27 mmol) in DMF (3.1 mL). The mixture was stirred at 30° C. for 3 h. The reaction at rt was quenched with 2M aq. NaH₂PO₄ (30 mL). The mixture was extracted twice with EtOAc/THF (ca. 5/1, 30 mL). The combined organic extracts were washed with brine, dried over Na₂SO₄, filtrated, and concentrated. The resulting residue was purified by silica gel flash chromatography (gradient, CH₂Cl₂/MeOH=100/0 to 92/8) to afford the title compound (3.32 g, 5.06 mmol). ¹H NMR (500 MHz, DMSO-d₆) δ 12.57 (br s, 1H), 7.89 (d, J=7.5 Hz, 2H), 7.72 (d, J=7.5 Hz, 2H), 7.61 (m, 1H), 7.42 (dd, J=7.0, 7.5 Hz, 2H), 7.33 (m, 6H), 7.23 (t, J=6.5 Hz, 1H), 6.45 (s, 1H), 4.27-4.20 (m, 3H), 3.89 (m, 1H), 3.25-3.19 (m, 6H), 3.01-2.97 (m, 2H), 2.42-2.38 (m, 4H), 1.72-1.56 (m, 8H), 1.69-1.56 (m, 2H), 1.39-1.29 (m, 2H) MS (ESI+): m/z 657.6 [M+H].

### Example-1-2

The following compounds were synthesized as outlined for the preparation of Example-1-2-01 employing appropriate starting materials in the table.

| | Structure and IUPAC name |
|---|---|
| | Fmoc-protected starting material-A |
| | Starting material-B |
| Example | NMR and MS |
| 1-2-02 | 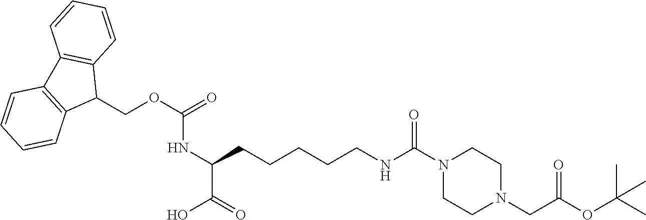 [SMILES: CC(C)(C)OC(=O)CN1CCN(C(=O)NCCCCC[C@H](NC(=O)OCC2c3ccccc3-c3ccccc32)C(=O)O)CC1] |
| | (2S)-7-({4-[2-(tert-butoxy)-2-oxoethyl]piperazine-1-carbonyl}amino)-2- |
| | ({[(9H-fluoren-9-yl)methoxy]carbonyl}amino)heptanoic acid |
| | Intermediate-1-3-02 |
| | tert-butyl 2-(piperazin-1-yl)acetate |
| | ¹H NMR (500 MHz, DMSO-d₆) δ 12.57 (br s, 1H), 7.89 (d, J = 7.5 Hz |
| | 2H), 7.73 (d, J = 7.5 Hz, 2H), 7.59 (m, 1H), 7.42 (dd, J = 7.5, 7.5 Hz, |
| | 2H), 7.33 (dd, J = 7.5, 7.5 Hz, 2H), 6.43 (s, 1H), 4.30-4.21 (m, 3H), |
| | 3.90-3.89 (m, 1H), 3.24 (m, 4H), 3.10 (s, 2H), 2.99-2.98 (m, 2H), 2.42 |
| | (m, 4H), 1.72-1.52 (m, 2H), 1.40 (s, 9H), 1.38-1.16 (m, 6H). MS |
| | (ESI+): m/z 609.6 [M + H]. |
| 1-2-03 | 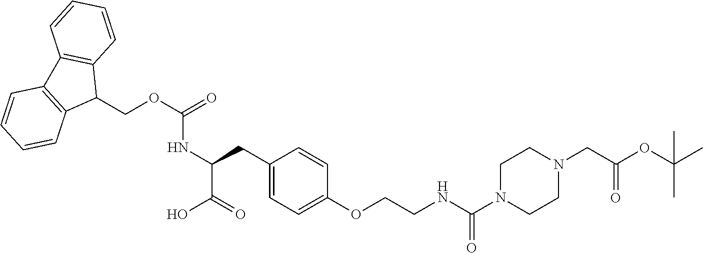 [SMILES: CC(C)(C)OC(=O)CN1CCN(C(=O)NCCOc2ccc(C[C@H](NC(=O)OCC3c4ccccc4-c4ccccc43)C(=O)O)cc2)CC1] |
| | (2S)-3-{4-[2-({4-[2-(tert-butoxy)-2-oxoethyl]piperazine-1-carbonyl}amino) |
| | ethoxy]phenyl}-2-({[(9H-fluoren-9-yl)methoxy]carbonyl}amino)propanoic |
| | acid |
| | Intermediate-1-3-03 |
| | tert-butyl 2-(piperazin-1-yl)acetate |
| | ¹H NMR (500 MHz, DMSO-d₆) δ 12.72 (br s, 1H), 7.88 (d, J = 7.5 Hz, |
| | 2H), 7.70-7.61 (m, 3H), 7.43-7.39 (m, 2H), 7.33-7.27 (m, |
| | 2H), 7.16 (d, J = 8.5 Hz, 2H), 6.84 (d, J = 8.5 Hz, 2H), 6.71 (t, J = |
| | 5.3 Hz, 1H), 4.23-4.05 (m, 4H), 3.92 (t, J = 6.0 Hz, 2H), 3.36-3.26 |
| | (m, 6H), 3.10 (s, 2H), 3.00 (dd, J = 13.5, 4.5 Hz, 1H), 2.79 (dd, |
| | J = 13.5, 10.5 Hz, 1H), 2.47-2.39 (m, 4H), 1.40 (s, 9H): MS(ESI+): |
| | m/z 673.5 [M + H]. |

### Example-1-3-01: (2R)-6-[({1-[2-(tert-butoxy)-2-oxoethyl]piperidin-4-yl}carbamoyl)amino]-2-({[(9H-fluoren-9-yl)methoxy]carbonyl}amino)hexanoic acid

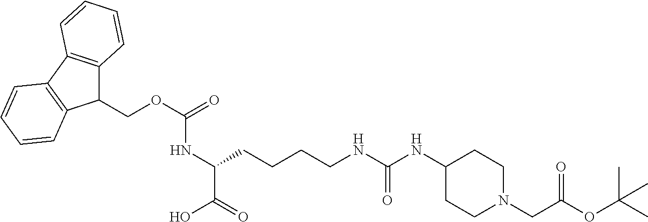 [SMILES: CC(C)(C)OC(=O)CN1CCC(NC(=O)NCCCC[C@@H](NC(=O)OCC2c3ccccc3-c3ccccc32)C(=O)O)CC1]

To a suspension of (2R)-6-amino-2-({[(9H-fluoren-9-yl)methoxy]carbonyl}amino)hexanoic acid hydrochloride (CAS: 201002-47-3, 1 g, 2.5 mmol) and Intermediate-1-01 (0.93 g, 2.72 mmol) in DMF (12.4 mL) at 0° C. was added DIPEA (0.48 mL, 2.72 mmol). The mixture was stirred at rt for 0.5 h, and then diluted with a mixture of EtOAc and hexane. The organic layer was washed with 1M aq. HCl. The aqueous layer was then extracted with a mixture of EtOAc and hexane. The combined organic extracts were washed with brine, dried over Na₂SO₄, filtered, and then concentrated. The resulting residue was purified by silica gel flash column chromatography (gradient, CH₂Cl₂/MeOH=100/0 to 80/20). Appropriate fractions were concentrated. To a solution of the resulting residue (1.21 g, 2.04 mmol) in CH₂Cl₂ (8 mL) at 0° C. was added 4 M HCl in dioxane (4 mL). The mixture was stirred at 0° C. for 6 h, and then concentrated. The resulting residue was suspended in toluene, and then concentrated. To the resulting residue in THF (10.2 mL) at 0° C. was added MSA (1.04 g, 7.12 mmol). The mixture was stirred at 0° C. for 0.25 h. To the reaction mixture at 0° C. was then added DIPEA (1.07 mL, 6.10 mmol), followed tert-butyl bromoacetate (CAS: 5292-43-3, 0.45 mL, 3.05 mmol). The mixture was then stirred at rt for 6 h. The reaction was quenched with a mixture of EtOAc and 0.5M aq. NaH₂PO₄ (20 mL). The mixture was extracted twice with EtOAc. The combined organic extracts were washed successively with H₂O and brine, dried over Na₂SO₄, filtered, and then concentrated. The resulting residue was purified by silica gel flash column chromatography (gradient, CH₂Cl₂/MeOH=100/0 to 80/20) to afford the title compound. ¹H NMR (500 MHz, DMSO-d₆) δ 12.58 (br s, 1H), 7.90 (d, J=7.5 Hz, 2H), 7.73 (d, J=7.3 Hz, 2H), 7.61 (br s, 1H), 7.42 (dd, J=7.4, 7.4 Hz, 2H), 7.33 (dd, J=7.4, 7.4 Hz, 2H), 5.71 (m, 2H), 4.31-4.20 (m, 3H), 3.92-3.85 (m, 1H), 3.32-3.27 (m, 1H), 3.04 (s, 2H), 3.00-2.90 (m, 2H), 2.71 (br d, J=10.8 Hz, 2H), 2.18 (br t, J=10.7 Hz, 2H), 1.69 (br d, J=11.0 Hz, 3H), 1.65-1.55 (m, 1H), 1.40 (s, 9H), 1.36-1.22 (m, 6H). MS (ESI+): m/z 609.5 [M+H].

### Example 1-3

The following compounds were synthesized as outlined for the preparation of Example-1-3-01 employing appropriate starting materials as indicated in the table.

| | Structure and IUPAC name |
|---|---|
| | Fmoc-protected starting material-A |
| | Starting material-B |
| Example | NMR and MS |
| 1-3-02 | 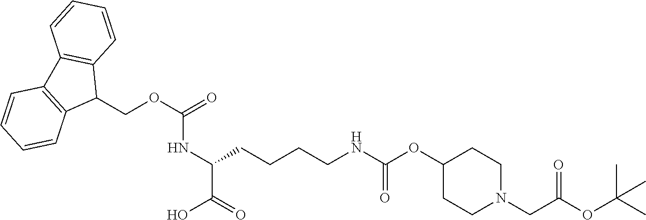 [SMILES: CC(C)(C)OC(=O)CN1CCC(OC(=O)NCCCC[C@@H](NC(=O)OCC2c3ccccc3-c3ccccc32)C(=O)O)CC1] |
| | (2R)-6-{[({1-[2-(tert-butoxy)-2-oxoethyl]piperidin-4-yl}oxy)carbonyl] |
| | amino}-2-({[(9H-fluoren-9-yl)methoxy]carbonyl}amino)hexanoic acid |
| | prop-2-en-1-yl (2R)-6-amino-2-({[(9H-fluoren-9-yl)methoxy]carbonyl} |
| | amino)hexanoate hydrochloride |
| | Intermediate-1-1-02 |
| | ¹H NMR (500 MHz, DMSO-d₆) δ 12.54 (br s, 1H), 7.90 (d, J = 7.6 Hz, |
| | 2H), 7.72 (d, J = 7.6 Hz, 2H), 7.62 (d, J = 7.8 Hz, 1H), 7.42 (t, J = 7.2 Hz, |
| | 2H), 7.33 (t, J = 7.3 Hz, 2H), 7.08 (s, 1H), 4.49-4.41 (m, 1H), |
| | 4.30-4.19 (m, 3H), 3.92-3.86 (m, 1H), 3.07 (s, 2H), 3.00-2.90 (m, |
| | 2H), 2.73-2.67 (m, 2H), 2.34-2.26 (m, 2H), 1.82-1.75 (m, 2H), 1.72- |
| | 1.64 (m, 1H), 1.62-1.56 (m, 1H), 1.54-1.44 (m, 2H), 1.40 (s, 9H), |
| | 1.38-1.27 (m, 4H). MS (ESI+): m/z 610.5 [M + H]. |

### Example-1-4-01: (2S)-6-({1-[2-(tert-butoxy)-2-oxoethyl]piperidin-4-yl}formamido)-2-({[(9H-fluoren-9-yl)methoxy]carbonyl}amino)hexanoic acid

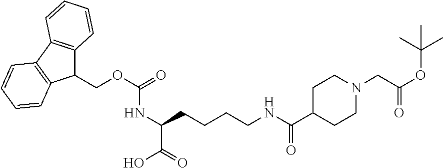 [SMILES: CC(C)(C)OC(=O)CN1CCC(C(=O)NCCCC[C@H](NC(=O)OCC2c3ccccc3-c3ccccc32)C(=O)O)CC1]

To a solution of prop-2-en-1-yl (2S)-6-amino-2-({[(9H-fluoren-9-yl)methoxy]carbonyl}amino)hexanoate hydrochloride (10.00 g, 22.47 mmol) in DMF (100 mL) at rt were added HATU (10.25 g, 26.97 mmol), DIPEA (5.81 g, 44.95 mmol) and 1-(tert-butoxycarbonyl)piperidine-4-carboxylic acid (CAS: 84358-13-4, 6.18 g, 26.97 mmol). The mixture was stirred at rt for 4 h, and then diluted with H₂O. The mixture was extracted twice with EtOAc. The combined organic extracts were washed three times with brine, dried over Na₂SO₄, filtered, and concentrated. The resulting residue was purified by silica gel flash column chromatography (isocratic, EtOAc/petroleum ether=1/1). Appropriate fractions were collected and concentrated. To the resulting residue in CH₂Cl₂ (80 mL) was added TFA (80 mL). The mixture was stirred at rt for 2 h, and then concentrated. To the resulting residue in DMF (100 mL) were added DIPEA (8.37 g, 64.76 mmol) and tert-butyl 2-bromoacetate (4.74 g, 24.29 mmol). The mixture was stirred at rt for ca. 16 h, and then diluted with H₂O. The mixture was extracted twice with EtOAc. The combined organic extracts were washed twice with brine, dried over Na₂SO₄, filtered, and then concentrated. The resulting residue was purified by silica gel flash column chromatography (gradient, petroleum ether/EtOAc=100/0 to 0/100).

Appropriate fractions were combined and concentrated. The resulting residue was dissolved in MeOH (100 mL), To a flask charged with Pd/C (2.0 g) under N₂ atmosphere at 0° C. was added the MeOH solution, followed by triethylsilane (18.35 g, 157.78 mmol). The mixture was stirred at rt for 1 h, and then filtered, and then concentrated. The resulting residue was purified by silica gel flash column chromatography (CH₂Cl₂/MeOH=92/8) to afford the title compound (6.32 g, 10.64 mmol). ¹H NMR (300 MHz, DMSO-d₆) δ 7.88 (d, J=7.5 Hz, 2H), 7.72 (d, J=7.7 Hz, 3H), 7.51-7.28 (m, 4H), 4.24 (q, J=6.1, 4.7 Hz, 3H), 3.88 (s, 1H), 3.16 (s, 2H), 3.02 (d, J=6.5 Hz, 4H), 2.78 (d, J=11.0 Hz, 2H), 2.19-1.92 (m, 3H), 1.78-1.48 (m, 6H), 1.39 (s, 13H). MS (ESI+): m/z 594.3 [M+H].

### Example-1-4-02: (2R)-6-{1-[2-(tert-butoxy)-2-oxoethyl]piperidine-4-sulfonamido}-2-({[(9H-fluoren-9-yl)methoxy]carbonyl}amino)hexanoic acid

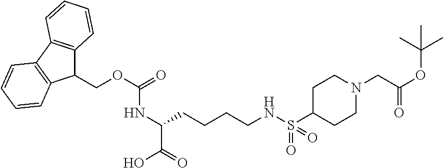 [SMILES: CC(C)(C)OC(=O)CN1CCC(S(=O)(=O)NCCCC[C@@H](NC(=O)OCC2c3ccccc3-c3ccccc32)C(=O)O)CC1]

To a solution of prop-2-en-1-yl (2R)-6-amino-2-({[(9H-fluoren-9-yl)methoxy]carbonyl}amino)hexanoate hydrochloride (2.0 g, 4.49 mmol) in CH₂Cl₂ (45 mL) at rt were added DIPEA (3.1 mL, 17.98 mmol) and tert-butyl 4-(chlorosulfonyl)piperidine-1-carboxylate (1.8 g, 6.29 mmol). The mixture was stirred for ca. 16 h at the same temperature and then diluted with H₂O. The mixture was extracted twice with CH₂Cl₂. The combined organic layer was washed with brine, dried over Na₂SO₄, filtered, and then concentrated. The resulting residue was purified by silica gel flash column chromatography (gradient, hexane/EtOAc=80/20 to 20/80). Appropriate fractions were concentrated. To a solution of the resulting residue (1.35 g, 2.06 mmol) in 1,4-dioxane (10.3 mL) was added 4M HCl in 1,4-dioxane (15.4 mL) at rt. The mixture was stirred for ca. 16 h at the same temperature and then concentrated. The resulting residue was used in the next step without any further purification. To a solution of the resulting residue in DMF (10.3 mL) at rt were added DIPEA (1.43 mL, 8.23 mmol) and tert-butyl 2-bromoacetate (0.60 g, 3.09 mmol). The mixture was stirred for ca. 16 h at the same temperature, and then diluted at 0° C. with H₂O. The mixture was extracted twice with EtOAc. The combined organic layer was washed twice with brine, dried over Na₂SO₄, filtered and concentrated. The resulting residue was purified by silica gel flash column chromatography (gradient, heptane/EtOAc=80/20 to 20/80). Appropriate fractions were concentrated. To a solution of the resulting residue (1.47 g, 2.20 mmol) in CH₂Cl₂ (29 mL) at 0° C. were added phenylsilane (0.63 g, 5.78 mmol) and Pd(PPh₃)₄ (0.08 g, 0.07 mmol). The mixture was stirred at 0° C. for 1 h, and then concentrated. The resulting residue was purified by silica gel flash column chromatography (gradient, CH₂Cl₂/MeOH=100/0 to 85/15) to afford the title compound (1.691 g, 2.69 mmol). ¹H NMR (500 MHz, DMSO-d₆) δ 12.73-12.52 (m, 1H), 7.90 (br d, J=7.5 Hz, 2H), 7.73 (br d, J=6.9 Hz, 2H), 7.66-7.59 (m, 1H), 7.42 (dd, J=7.4, 7.4 Hz, 2H), 7.33 (t, J=7.3 Hz, 2H), 7.05 (t, J=5.8 Hz, 1H), 4.31-4.21 (m, 3H), 3.94-3.87 (m, 1H), 3.09 (s, 2H), 2.96-2.83 (m, 5H), 2.19 (br t, J=11.2 Hz, 2H), 1.89 (br d, J=11.0 Hz, 2H), 1.73-1.51 (m, 4H), 1.49-1.27 (m, 4H), 1.40 (s, 9H). MS (ESI+): m/z 63.4 [M+H].

### Example-1-4-03: (2S)-3-[4-(2-{4-[2-(tert-butoxy)-2-oxoethyl]piperazin-1-yl}-2-oxoethoxy)phenyl]-2-({[(9H-fluoren-9-yl)methoxy]carbonyl}amino)propanoic acid

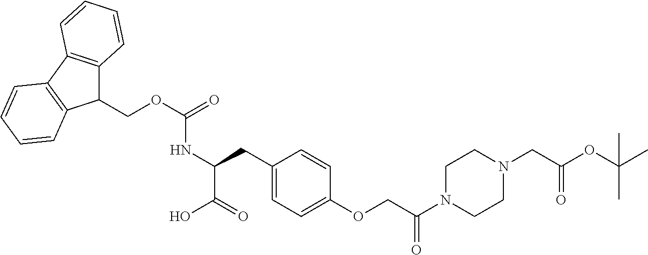 [SMILES: CC(C)(C)OC(=O)CN1CCN(C(=O)COc2ccc(C[C@H](NC(=O)OCC3c4ccccc4-c4ccccc43)C(=O)O)cc2)CC1]

To a solution of (2S)-3-{4-[2-(tert-butoxy)-2-oxoethoxy]phenyl}-2-({[(9H-fluoren-9-yl)methoxy]carbonyl}amino)propanoic acid (CAS: 181951-92-8, 40 g, 77.28 mmol) in DMF (400 mL) at 0° C. were added NaHCO₃ (13.00 g, 154.57 mmol) and 3-bromoprop-1-ene (11.20 g, 92.74 mmol). The mixture was stirred at rt for 2 h, and then diluted with H₂O. The mixture was extracted twice with EtOAc. The combined organic extracts were washed three times with brine, dried over Na₂SO₄, filtrated, and then concentrated. The resulting residue was purified by silica gel column chromatography (petroleum ether/EtOAc=50/50). Appropriate fractions were concentrated. To a solution of the resulting residue (31 g, 55.60 mmol) in CH₂Cl₂ (300 mL) at 0° C. was added TFA (150 mL). The mixture was stirred at rt for 2 h, and then concentrated. The resulting residue was suspended in toluene, and then concentrated. To a solution of the resulting residue in DMF (400 mL) at rt were added tert-butyl 2-(piperazin-1-yl)acetate (13.609 g, 67.983 mmol), HATU (47.00 g, 123.63 mmol), and DIPEA (31.93 g, 247.26 mmol). The mixture was stirred at rt for 3 h, and then diluted with H₂O. The mixture was extracted three times with EtOAc. The combined organic extracts were washed with three times with brine, dried over Na₂SO₄, filtered, and then concentrated. The resulting residue was purified by silica gel flash column chromatography (petroleum ether/EtOAc=50/50). Appropriate fractions were concentrated. To a solution of the resulting residue (23 g, 33.64 mmol) in THF at rt were added morpholine (5.86 g, 67.26 mmol) and Pd(PPh₃)₄ (3.89 g, 3.36 mmol). The mixture was stirred at rt for 0.5 h. The reaction was quenched with H₂O. The pH of the mixture was adjusted to 6 with citric acid. The mixture was extracted three times with EtOAc. The combined organic extracts were washed three times with brine, dried over Na₂SO₄, filtrated, and then concentrated. The resulting residue was purified by reverse-phase flash chromatography (Column, C18 silica gel; gradient, CH₃CN/H₂O=10/90 to 50/50) to afford the title compound (11.0 g, 17.1 mmol). ¹H NMR (400 M Hz, DMSO-d₆) δ 12.68 (br s, 1H), 7.88 (d, J=7.2 Hz, 2H), 7.73-7.37 (m, 8H), 7.36-7.24 (m, 2H), 7.17 (d, J=8.4 Hz, 2H), 6.90-6.75 (m, 2H), 4.73 (s, 2H), 4.25-4.06 (m, 4H), 3.61 (br s, 1H), 3.43 (br s, 4H), 3.14 (s, 2H), 3.01 (dd, J=13.8, 4.0 Hz, 1H), 2.80 (dd, J=13.8, 10.4 Hz, 1H), 1.40 (s, 9H). MS (ESI+): m/z 644.2 [M+H].

### Example-1-4

The following compounds were synthesized as outlined for the preparation of Example-1-4-01, Example-1-4-02, or Example-1-4-03 employing appropriate starting materials in the table.

| | Structure and IUPAC name |
|---|---|
| | Fmoc-protected starting material-A |
| | Starting material-B |
| Example | NMR and MS |
| 1-4-04 | 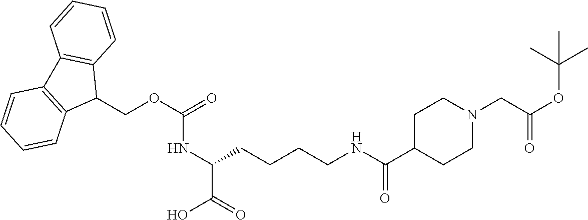 [SMILES: CC(C)(C)OC(=O)CN1CCC(C(=O)NCCCC[C@@H](NC(=O)OCC2c3ccccc3-c3ccccc32)C(=O)O)CC1] |
| | (2R)-6-({1-[2-(tert-butoxy)-2-oxoethyl]piperidin-4-yl}formamido)-2-({[(9H- |
| | fluoren-9-yl)methoxy]carbonyl}amino)hexanoic acid |
| | prop-2-en-1-yl (2R)-6-amino-2-({[(9H-fluoren-9-yl)methoxy]carbonyl} |
| | amino)hexanoate hydrochloride |
| | 1-(tert-butoxycarbonyl)piperidine-4-carboxylic acid |
| | Substantially identical with Example-1-4-01. |
| 1-4-05 | 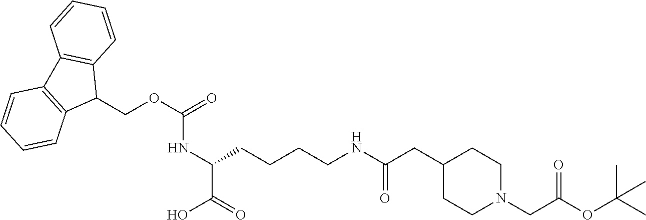 [SMILES: CC(C)(C)OC(=O)CN1CCC(CC(=O)NCCCC[C@@H](NC(=O)OCC2c3ccccc3-c3ccccc32)C(=O)O)CC1] |
| | (2R)-6-(2-{1-[2-(tert-butoxy)-2-oxoethyl]piperidin-4-yl}acetamido)-2- |
| | ({[(9H-fluoren-9-yl)methoxy]carbonyl}amino)hexanoic acid |
| | prop-2-en-1-yl (2R)-6-amino-2-({[(9H-fluoren-9-yl)methoxy]carbonyl} |
| | amino)hexanoate hydrochloride |
| | 2-(1-(tert-butoxycarbonyl)piperidin-4-yl)acetic acid |
| | ¹H NMR (500 MHz, DMSO-d₆) δ 12.60 (br s, 1H), 7.90 (d, J = 7.3 Hz, |
| | 2H), 7.78 (m, 1H), 7.72 (d, J = 7.5 Hz, 2H), 7.42 (dd, J = 7.2, 7.2 Hz, |
| | 2H), 7.36-7.30 (dd, J = 7.2, 7.2 Hz, 2H), 4.30-4.19 (m, 3H), 3.91- |
| | 3.79 (m, 1H), 3.02 (m, 4H), 2.75 (d, J = 10.2 Hz, 2H), 2.08 (t, J = 11.2 Hz, |
| | 2H), 1.95 (d, J = 6.6 Hz, 2H), 1.73-1.63 (m, 1H), 1.59 (br s, 2H), |
| | 1.53 (d, J = 13.7 Hz, 2H), 1.39 (s, 9H), 1.37-1.22 (m, 4H), 1.20- |
| | 1.05 (m, 2H), MS (ESI+): m/z 608.5 [M + H]. |
| 1-4-06 | 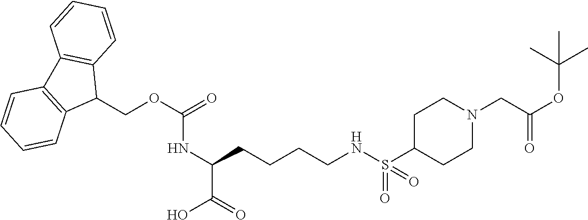 [SMILES: CC(C)(C)OC(=O)CN1CCC(S(=O)(=O)NCCCC[C@H](NC(=O)OCC2c3ccccc3-c3ccccc32)C(=O)O)CC1] |
| | (2S)-6-{1-[2-(tert-butoxy)-2-oxoethyl]piperidine-4-sulfonamido}-2-({[(9H- |
| | fluoren-9-yl)methoxy]carbonyl}amino)hexanoic acid |
| | prop-2-en-1-yl (2S)-6-amino-2-({[(9H-fluoren-9-yl)methoxy]carbonyl} |
| | amino)hexanoate hydrochloride |
| | tert-butyl 4-(chlorosulfonyl)piperidine-1-carboxylate |
| | Substantially identical to Example-1-4-02 |
| 1-4-07 | 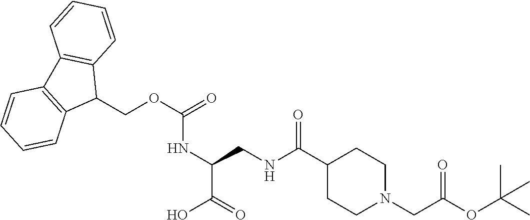 [SMILES: CC(C)(C)OC(=O)CN1CCC(C(=O)NC[C@H](NC(=O)OCC2c3ccccc3-c3ccccc32)C(=O)O)CC1] |
| | (2S)-3-({1-[2-(tert-butoxy)-2-oxoethyl]piperidin-4- |
| | yl}formamido)-2-({[(9H-fluoren-9- |
| | yl)methoxy]carbonyl}amino)propanoic acid |
| | prop-2-en-1-yl (2S)-3-amino-2-({[(9H-fluoren-9-yl)methoxy]carbonyl} |
| | amino)propanoate hydrochloride |
| | 1-(tert-butoxycarbonyl)piperidine-4-carboxylic acid |
| | ¹H NMR (500 MHz, CDCl₃) δ 7.80-7.67 (m, 2H), 7.67-7.53 (m, 2H), |
| | 7.41-7.32 (m, 2H), 7.32-7.24 (m, 2H), 7.16 (br s, 1H), 6.53 (br s, |
| | 1H), 4.52-4.08 (m, 4H), 3.93-3.65 (m, 2H), 3.43-3.15 (m, 4H), 2.63 |
| | (br s, 2H), 2.29 (m, 1H), 2.07-1.74 (m, 4H), 1.42 (s, 9H). MS (ESI+): |
| | m/z 552.5 [M + H]. |
| 1-4-08 | 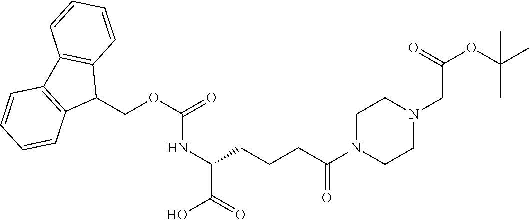 [SMILES: CC(C)(C)OC(=O)CN1CCN(C(=O)CCC[C@@H](NC(=O)OCC2c3ccccc3-c3ccccc32)C(=O)O)CC1] |
| | (2R)-6-{4-[2-(tert-butoxy)-2-oxoethyl]piperazin-1-yl}-2-({[(9H-fluoren-9- |
| | yl)methoxy]carbonyl}amino)-6-oxohexanoic acid |
| | Intermediate-1-2-01 |
| | tert-butyl 2-(piperazin-1-yl)acetate |
| | ¹H NMR (500 MHz, DMSO-d₆) δ 12.60 (br s, 1H), 7.90 (d, J = 7.5 Hz, |
| | 2H), 7.73 (d, J = 7.5 Hz, 2H), 7.71-7.50 (m, 1H), 7.42 (dd, J = 7.5, |
| | 7.5 Hz, 2H), 7.33 (dd, J = 7.5, 7.5 Hz, 2H), 4.27-4.21 (m, 3H), 3.94- |
| | 3.89 (m, 1H), 3.42-3.39 (m, 4H), 3.12 (s, 2H), 2.50-2.45 (m, 2H), |
| | 2.44-2.40 (m, 2H), 2.33-2.24 (m, 2H), 1.74-1.71 (m, 1H), 1.62- |
| | 1.52 (m, 3H), 1.40 (s, 9H). MS (ESI+): m/z 566.5 [M + H]. |
| 1-4-09 | 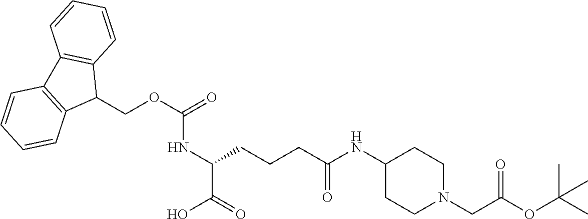 [SMILES: CC(C)(C)OC(=O)CN1CCC(NC(=O)CCC[C@@H](NC(=O)OCC2c3ccccc3-c3ccccc32)C(=O)O)CC1] |
| | (2R)-5-({1-[2-(tert-butoxy)-2-oxoethyl]piperidin-4-yl}carbamoyl)-2-({[(9H- |
| | fluoren-9-yl)methoxy]carbonyl}amino)pentanoic acid |
| | Intermediate-1-2-01 |
| | tert-butyl 4-aminopiperidine-1-carboxylate (CAS: 87120-72-7) |
| | ¹H NMR (500 MHz, DMSO-d₆) δ 12.55 (br s, 1H), 7.89 (d, J = 7.5 Hz, |
| | 2H), 7.72 (d, J = 7.5 Hz, 3H), 7.42 (dd, J = 7.5, 7.5 Hz, 2H), 7.33 (dd, |
| | J = 7.5, 7.5 Hz, 2H), 4.26-4.21 (m, 3H), 3.90-3.82 (m, 1H), 3.52- |
| | 3.43 (m, 1H), 3.06 (s, 2H), 2.75 (d, J = 11 Hz, 2H), 2.17 (t, J = 11.5 Hz, |
| | 2H), 2.04-2.02 (m, 2H), 1.69-1.63 (m, 3H), 1.57-1.47 (m, 3H), |
| | 1.42-1.30 (m, 11H). MS (ESI+): m/z 580.5 [M + H]. |

### Example-1-5-01: (2R)-6-{[({1,3-bis[(2,2-dimethyl-1,3-dioxan-5-yl)methoxy]propan-2-yl}oxy)carbonyl]amino}-2-({[(9H-fluoren-9-yl)methoxy]carbonyl}amino)hexanoic acid

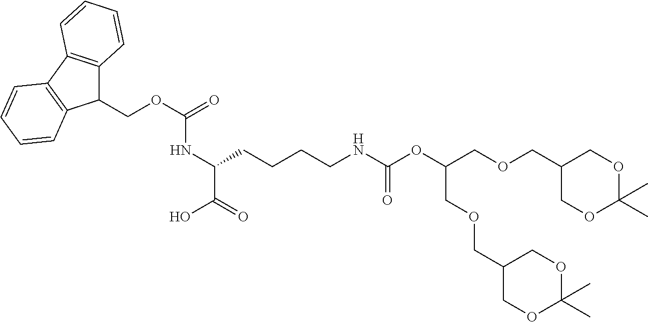 [SMILES: CC1(C)OCC(COCC(COCC2COC(C)(C)OC2)OC(=O)NCCCC[C@@H](NC(=O)OCC2c3ccccc3-c3ccccc32)C(=O)O)CO1]

To a solution of Intermediate-1-04 (0.41 mg, 1.17 mmol) in acetonitrile (3.9 mL) at rt were added bis(2,5-dioxopyrrolidin-1-yl) carbonate (895 mg, 3.50 mmol) and DIPEA (814 μl, 4.66 mmol). The mixture was stirred for ca. 16 h at the same temperature, and then poured into a mixture of silica gel and Na₂SO₄ in EtOAc. The mixture was filtered, and then concentrated. The resulting residue was purified by silica gel flash column chromatography (gradient, hexane/EtOAc=50/50 to 0/100). The obtained compound was diluted in CH₂Cl₂. The white precipitate was filtered out and the filtrate was concentrated. To a solution of resulting residue in CH₂Cl₂ (3.88 mL) were added DIPEA (509 μl, 2.91 mmol) and prop-2-en-1-yl (2R)-6-amino-2-({[(9H-fluoren-9-yl)methoxy]carbonyl}amino)hexanoate hydrochloride (518 mg, 1.165 mmol) at rt. The mixture was stirred for 1 h at the same temperature and added H₂O. The mixture was extracted twice with EtOAc. The combined organic extracts were washed with brine, dried over Na₂SO₄, filtered and concentrated. The resulting residue was purified by silica gel flash column chromatography (hexane/EtOAc=50/50 to 0/1). Appropriate fractions were concentrated. To a resulting residue (707 mg, 0.903 mmol) in CH₂Cl₂ (9 mL) were added phenylsilane (195 mg, 1.806 mmol) and Pd(PPh₃)₄ (26.1 mg, 0.023 mmol) at 0° C. The mixture was stirred for 1.5 h at the same temperature and then concentrated. The resulting residue was purified by silica gel flash column chromatography (CH₂Cl₂/MeOH=100/0 to 85/15) to afford the title compound (525 mg, 0.707 mmol). ¹H NMR (500 MHz, CDCl₃) δ 7.77 (d, J=7.5 Hz, 2H), 7.61 (d, J=7.5 Hz, 2H), 7.40 (dd, J=7.5 Hz, 2H), 7.32 (dd, J=7.5 Hz, 2H), 5.57-5.52 (m, 1H), 5.07-4.95 (m, 1H), 4.70-4.62 (m, 1H), 4.52-4.39 (m, 3H), 4.24-4.20 (m, 1H), 3.99-3.95 (m, 4H), 3.77-3.72 (m, 4H), 3.64-3.45 (m, 6H), 3.29-3.12 (m, 2H), 2.05-1.23 (m, 20H).

### Example-1-5

The following compounds were synthesized as outlined for the preparation of Example-1-5-01 employing appropriate starting materials in the table.

| | Structure and IUPAC name |
|---|---|
| | Fmoc-protected starting material-A |
| | Starting material-B |
| Example | NMR and MS |
| 1-5-02 | 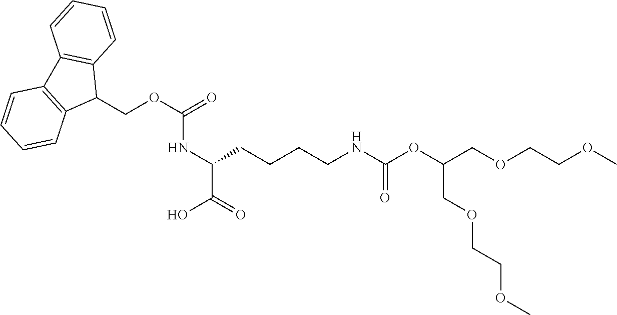 [SMILES: COCCOCC(COCCOC)OC(=O)NCCCC[C@@H](NC(=O)OCC1c2ccccc2-c2ccccc21)C(=O)O] |
| | (2R)-2-({[(9H-fluoren-9-yl)methoxy]carbonyl}amino)-6-{4-[(2-methoxy- |
| | ethoxy)methyl]-2-oxo-3,6,9-trioxa-1-azadecan-1-yl}hexanoic acid |
| | prop-2-en-1-yl (2R)-6-amino-2-({[(9H-fluoren-9-yl)methoxy]carbonyl} |
| | amino)hexanoate hydrochloride |
| | Intermediate-1-1-05 |
| | ¹H NMR (500 MHz, CDCl₃) δ 7.77 (d, J = 5 Hz, 2H), 7.61 (d, J = 7.5 Hz, |
| | 2H), 7.40 (dd, J = 7.5 Hz, 2H), 7.32 (dd, J = 7.5 Hz, 2H), 5.57- |
| | 5.52 (m, 1H), 5.05-4.98 (m, 1H), 4.79-4.71 (m, 1H), 4.52-4.38 |
| | (m, 3H), 4.26-4.20 (m, 1H), 3.71-3.52 (m, 12H), 3.42-3.37 (m, 6H), |
| | 3.36-3.15 (m, 2H), 1.86-1.35 (m, 6H). MS (ESI+): m/z 603.5 |
| 1-5-03 | 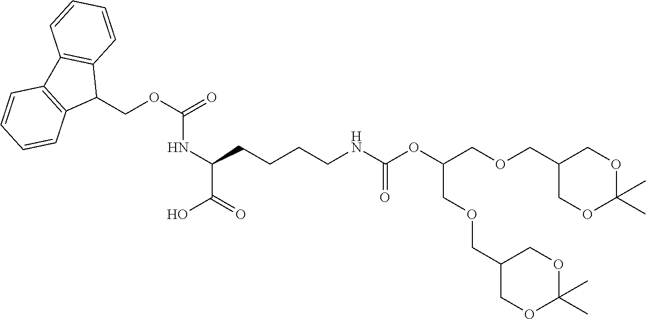 [SMILES: CC1(C)OCC(COCC(COCC2COC(C)(C)OC2)OC(=O)NCCCC[C@H](NC(=O)OCC2c3ccccc3-c3ccccc32)C(=O)O)CO1] |
| | (2S)-6-{[({1,3-bis[(2,2-dimethyl-1,3-dioxan-5-yl)methoxy]propan-2-yl} |
| | oxy)carbonyl]amino}-2-({[(9H-fluoren-9-yl)methoxy]carbonyl}amino) |
| | hexanoic acid |
| | prop-2-en-1-yl (2S)-6-amino-2-({[(9H-fluoren-9-yl)methoxy]carbonyl} |
| | amino)hexanoate hydrochloride |
| | Intermediate-1-1-04 |
| | Substantially identical to Example-1-5-01 |

### Example-1-6-01: (2S)-6-({5-[(tert-butoxy)carbonyl]pyridin-2-yl}amino)-2-({[(9H-fluoren-9-yl)methoxy]carbonyl}amino)hexanoic acid

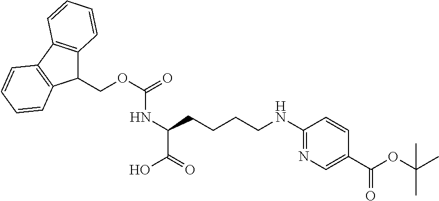 [SMILES: CC(C)(C)OC(=O)c1ccc(NCCCC[C@H](NC(=O)OCC2c3ccccc3-c3ccccc32)C(=O)O)nc1]

To a suspension of (2S)-6-({[(9H-fluoren-9-yl)methoxy]carbonyl}amino)-2-{[(prop-2-en-1-yloxy)carbonyl]amino}hexanoic acid (3.0 g, 6.63 mmol) in DMF (5.5 mL) at rt were added tert-butyl 6-fluoronicotinate (1.09 g, 5.52 mmol) and K₂CO₃ (2.29 g, 16.57 mmol). The mixture was stirred for 3 h at 80° C. and then cooled to rt. The mixture was diluted with H₂O and washed with EtOAc. pH of aqueous phase was adjusted to 4-5 with 10% aq. citric acid. The aqueous phase was extracted five times with CH₂Cl₂. The combined organic layer was washed twice with H₂O, brine, dried over Na₂SO₄, filtered, and concentrated. Appropriate fractions were concentrated. To a resulting residue (1.26 g, 3.10 mmol) in CH₂Cl₂ (16 mL) at rt were added phenylsilane (1.15 mL, 9.33 mmol) and Pd(PPh₃)₄ (0.36 g, 0.310 mmol). The mixture was stirred for 0.5 h at the same temperature and then concentrated. To the resulting residue in THF (7.7 mL) at rt were added NaHCO₃ (0.26 g, 3.10 mmol) in H₂O (7.7 mL) and Fmoc-OSu (0.99 g, 2.95 mmol). The mixture was stirred for 3 h at the same temperature. pH of the solution was adjusted to ca. 4.5 with 1M aq. HCl. The mixture was extracted three times with EtOAc. The combined organic extracts were washed with H₂O and brine, dried over Na₂SO₄, filtered and concentrated. The resulting residue was purified by silica gel flash column chromatography (gradient, hexane/EtOAc=100/0 to 0/100 then CH₂Cl₂/MeOH=100/0 to 70/30) to afford the title compound (1.07 g, 1.961 mmol). ¹H NMR (500 MHz, DMSO-d₆) δ 12.59 (br s, 1H), 8.42 (br s, 1H), 7.90 (d, J=7.5 Hz, 2H), 7.84 (br-s, 1H), 7.72 (br d, J=5.5 Hz, 2H), 7.68-7.61 (m, 2H), 7.42 (dd, J=7.5, 7.5 Hz, 2H), 7.32 (dd, J=7.5, 7.5 Hz, 2H), 6.64 (br s, 1H), 4.29-4.18 (m, 1H), 3.97-3.91 (m, 1H), 3.31 (br s, 2H), 1.78-1.69 (m, 1H), 1.69-1.59 (m, 1H), 1.59-1.48 (m, 11H), 1.46-1.32 (m, 2H). MS (ESI+): m/z 546.6 [M+H].

### Example-1-7-01: (2S)-2-({[(9H-fluoren-9-yl)methoxy]carbonyl}(methyl)amino)-6-{[(prop-2-en-1-yloxy)carbonyl]amino}hexanoic acid

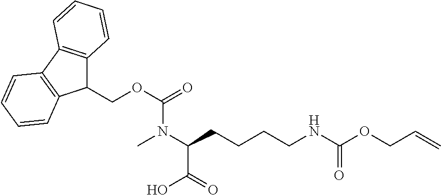 [SMILES: C=CCOC(=O)NCCCC[C@@H](C(=O)O)N(C)C(=O)OCC1c2ccccc2-c2ccccc21]

To a solution of (2S)-6-[(tert-butoxycarbonyl)amino]-2-[[(9H-fluoren-9-ylmethoxy)carbonyl](methyl)amino]hexanoic acid (65.0 g, 135 mmol) in CH₂Cl₂ (163 mL) at rt was added 3M HCl in CPME. The mixture was stirred for 1 h at rt, and then concentrated. To a solution of the resulting residue in THF/H₂O (780 mL/390 mL) at rt was added NaHCO₃ (28.6 g, 339.9 mmol), followed by allyl chloroformate (CAS: 2937-50-0, 22.5 g, 187.0 mmol). The mixture was stirred for 2 h at the same temperature. The pH of the solution was adjusted to 5 with citric acid. The resulting mixture was extracted three times with EtOAc. The combined organic extracts were washed with brine three times, dried over Na₂SO₄, filtered, and concentrated. The resulting residue was purified by RP-HPLC (Column, C18 silica gel; gradient, CH₃CN/H₂O with 0.05% NH₃=0/100 to 50/50) to afford title compound (50.1 g, 108 mmol). ¹H NMR (500 MHz, DMSO-d₆) δ 12.75 (br s, 1H), 7.94-7.85 (m, 2H), 7.69-7.58 (m, 2H), 7.46-7.37 (m, 2H), 7.35-7.26 (m, 2H), 7.23-7.13 (m, 1H), 5.96-5.83 (m, 1H), 5.25 (d, J=17.5 Hz, 1H), 5.15 (d, J=10.5 Hz, 1H), 4.53-4.25 (m, 6H), 3.02-2.90 (m, 2H), 2.78-2.67 (m, 3H), 1.88-0.96 (m, 6H). MS (ESI+): m/z 467.4 [M+H].

### Example-1-7

The following compounds were synthesized as outlined for the preparation of Example-1-7-01 employing appropriate starting materials in the table.

| | Structure and IUPAC name |
|---|---|
| | Fmoc-protected starting material-A |
| | Starting material-B |
| Example | NMR and MS |
| 1-7-02 | 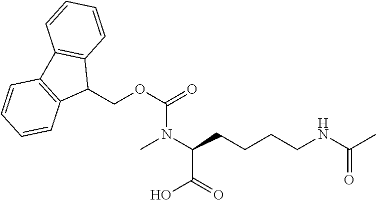 [SMILES: CC(=O)NCCCC[C@@H](C(=O)O)N(C)C(=O)OCC1c2ccccc2-c2ccccc21] |
| | (2S)-6-acetamido-2-({[(9H-fluoren-9- |
| | yl)methoxy]carbonyl}(methyl)amino)hexanoic acid (2S)- |
| | 6-[(tert-butoxycarbonyl)amino]-2-[[(9H-fluoren-9-ylmethoxy) |
| | carbonyl](methyl)amino]hexanoic acid (CAS: 197632-76-1) |
| | acetic anhydride |
| | ¹H NMR (500 MHz, DMSO-d₆) δ 12.95-12.74 (m, 1H), 7.93- |
| | 7.87 (m, 2H), 7.80 (br s, 1H), 7.68-7.59 (m, 2H), 7.46-7.38 |
| | (m, 2H), 7.36-7.29 (m, 2H), 4.68-4.20 (m, 4H), 3.04-2.95 |
| | (m, 2H), 2.75-2.70 (m, 3H), 1.87-1.51 (m, 5H), 1.44-1.29 |
| | (m, 2H), 1.21-1.03 (m, 2H). MS (ESI+): m/z 425.3 [M + H]. |

### Example-1-8-01: (2S)-2-({[(9H-fluoren-9-yl)methoxy]carbonyl}(methyl)amino)-4-{[(prop-2-en-1-yloxy)carbonyl]amino}butanoic acid

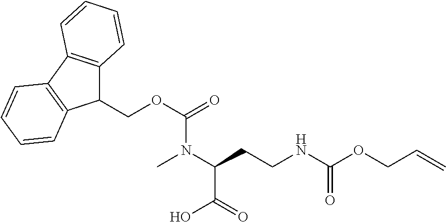 [SMILES: C=CCOC(=O)NCC[C@@H](C(=O)O)N(C)C(=O)OCC1c2ccccc2-c2ccccc21]

To a solution of (S)-3-(3-(((9H-fluoren-9-yl)methoxy)carbonyl)-5-oxooxazolidin-4-yl)propanoic acid (CAS: 159530-17-3, 3.81 g, 10.0 mmol) in THF (25 mL) at −20° C. were added 4-methylmorpholine (CAS: 109-02-4, 1.21 mL, 11.0 mmol) and isobutyl chloroformate (1.45 mL, 11.0 mmol) while maintaining the temperature below −15° C. The mixture was stirred at −15° C. for 20 min. To the mixture was added a solution of NaN₃ (0.98 g, 15.0 mmol) in H₂O (25 mL) while maintaining the temperature below 0° C. After stirring at rt for 2 h, the reaction was quenched at rt with H₂O. The mixture was extracted twice with EtOAc. The combined organic extracts were washed successively with H₂O, 1M aq. HCl, twice with satd. aq. NaHCO₃ and brine, dried over Na₂SO₄, filtered, and then concentrated. To a solution of the resulting residue in toluene (40 mL) at rt was added 2-methylpropan-2-ol (23.9 mL, 250 mmol). The mixture was stirred at 100° C. for 3 h, and then concentrated. The resulting residue was purified by silica gel flash column chromatography (gradient, hexane/EtOAc=100/0 to 0/100). Appropriate fractions were concentrated. To the resulting residue (0.27 g, 0.61 mmol) in ClCH₂CH₂Cl (1.5 mL) at 0° C. was added triethylsilane (0.73 mL, 1.82 mmol), followed by TFA (1.5 mL). The mixture was then stirred at 40° C. for ca. 16 h and then concentrated. The resulting residue was suspended in toluene, and then concentrated. To a solution of the resulting residue in DMF (2.4 mL) at 0° C. were added allyl (2,5-dioxopyrrolidin-1-yl) carbonate (0.12 g, 0.61 mmol) and DIPEA (0.32 mL, 1.82 mmol) while maintaining the temperature below 3° C. The mixture was stirred for 1 h at rt. The reaction was then quenched with 1M aq. HCl. The mixture was extracted with iPrOAc. The organic phase was washed successively with H₂O and brine, dried over Na₂SO₄, filtered, and concentrated. The resulting residue was triturated with iPrOAc and heptane. The resulting solid was collected by filtration to afford the title compound (0.26 g, 0.61 mmol). ¹H NMR (500 MHz, DMSO-d₆) δ 12.87 (br s, 1H), 7.91-7.88 (m, 2H), 7.64 (dd, J=18.0, 7.5 Hz, 2H), 7.43-7.41 (m, 2H), 7.36-7.20 (m, 3H), 6.04-5.79 (m, 1H), 5.48-5.10 (m, 2H), 4.52-4.18 (m, 6H), 3.03-2.89 (m, 2H), 2.81-2.69 (m, 3H), 2.09-1.98 (m, 1H), 1.89-1.74 (m, 1H); MS (ESI+): m/z 439.3 [M+H].

### Example-1-8-02: (2S)-3-acetamido-2-({[(9H-fluoren-9-yl)methoxy]carbonyl}(methyl)amino)propanoic acid

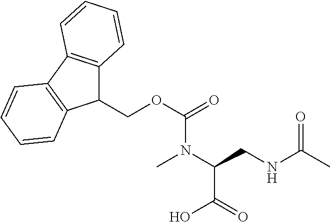 [SMILES: CC(=O)NC[C@@H](C(=O)O)N(C)C(=O)OCC1c2ccccc2-c2ccccc21]

To a mixture of (2S)-2-[[(9H-fluoren-9-ylmethoxy)carbonyl] amino]butanedioic acid (18.00 g, 50.66 mmol) in AcOH (200 mL) at rt were added paraformaldehyde (CAS: 30525-89-4, 15.20 g, 506.55 mmol) and CSA (1.18 g, 5.07 mmol). The mixture was stirred for at 110° C. 1 h and then concentrated. The resulting residue was diluted with EtOAc and H₂O. The mixture was extracted three times with EtOAc. The combined organic extracts were washed twice with brine, dried over Na₂SO₄, filtered, and concentrated. The resulting residue was purified by silica gel flash column chromatography (gradient, petroleum ether/EtOAc=50/50). Appropriate fractions were concentrated. To the resulting residue (18 g, 49.00 mmol) in THF (180 mL) at −10° C. were added 4-methylmorpholine (5.4 mL, 49.00 mmol) and Isobutyl chloroformate (6.61 mL, 49.00 mmol). The mixture was stirred at −10° C. for 0.25 h. To the mixture at −10° C. was added a solution of NaN₃ (4.78 g, 73.50 mmol) in H₂O (50 mL). The mixture was stirred at rt for 0.5 h. The reaction was quenched with H₂O. The mixture was extracted with EtOAc. The organic extract was washed successively with brine twice, 5% aq. HCl, 5% aq. NaHCO₃, and then dried over Na₂SO₄, filtered, and then concentrated. The resulting residue and toluene (175 mL) was then stirred for at 110° C. 1 h, and subsequently tert-BuOH (50 mL) was added to the mixture. The mixture was stirred at 110° C. for 1 h, and then concentrated. The resulting residue was purified by silica gel flash column chromatography (gradient, petroleum ether/EtOAc=100/0 to 75/25). Appropriate fractions were concentrated (15 g, 34.21 mmol). A mixture of the obtained material (9 g, 20.53 mmol) in CHCl₃/TFA/triethylsilane (90 mL/90 mL/90 mL) was stirred at 75° C. for 16 h. The mixture was concentrated. To a mixture of the resulting residue in THF (40 mL) at rt were added pyridine (1.86 g, 23.50 mmol) and acetic anhydride (2.40 g, 23.50 mmol). The mixture was stirred at rt for 1 h. The reaction was quenched with H₂O (100 mL). The mixture was extracted three times with EtOAc. The combined organic extracts were washed twice with brine, dried over Na₂SO₄, filtered, and concentrated. The resulting residue was purified by RP-HPLC (Column, C18 silica gel; gradient, CH₃CN/H₂O=5/95 to 100/0) to afford the title compound (2.27 g, 5.94 mmol). ¹H NMR (300 MHz, DMSO-d₆) δ 13.04 (s, 1H), 8.02-7.91 (m, 3H), 7.65 (dd, J=11.0, 7.4 Hz, 2H), 7.44-7.27 (m, 4H), 4.80-4.61 (m, 1H), 4.30-4.15 (m, 3H), 3.85-3.62 (m, 1H), 3.45-3.35 (m, 1H), 2.95-2.80 (m, 3H), 1.85-1.78 (m, 3H). MS (ESI+): m/z 383.2 [M+H].

### Example-1-8-03: (2S)-2-({[(9H-fluoren-9-yl)methoxy]carbonyl}(methyl)amino)-3-{[(prop-2-en-1-yloxy)carbonyl]amino}propanoic acid

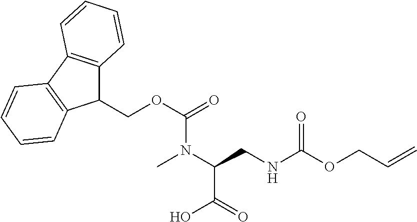 [SMILES: C=CCOC(=O)NC[C@@H](C(=O)O)N(C)C(=O)OCC1c2ccccc2-c2ccccc21]

The title compound was synthesized analogously to the preparation of Example-1-8-02 by using allyl chloroformate in instead of acetic anhydride. ¹H NMR (500 MHz, DMSO-d₆) δ 13.08 (br s, 1H), 7.94-7.87 (m, 2H), 7.69-7.57 (m, 2H), 7.53-7.28 (m, 5H), 5.95-5.79 (m, 1H), 5.31-5.04 (m, 2H), 4.86-4.57 (m, 1H), 4.54-4.39 (m, 2H), 4.33-4.15 (m, 3H), 3.72-3.35 (m, 2H), 2.91-2.73 (m, 3H). MS (ESI+): m/z 425.3 [M+H].

### Example-1-9-01: (2S)-3-{1-[2-(tert-butoxy)-2-oxoethyl]piperidin-4-yl}-2-({[(9H-fluoren-9-yl)methoxy]carbonyl}amino)propanoic acid

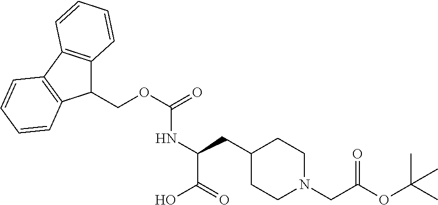 [SMILES: CC(C)(C)OC(=O)CN1CCC(C[C@H](NC(=O)OCC2c3ccccc3-c3ccccc32)C(=O)O)CC1]

To a solution of (S)-2-((((9H-fluoren-9-yl)methoxy)carbonyl)amino)-3-(1-(tert-butoxycarbonyl)piperidin-4-yl)propanoic acid (7.5 g, 15.16 mmol) in CH₂Cl₂ (60 mL) at 0° C. was added 4M hydrogen chloride in CPME (20 mL, 80 mmol). The mixture was stirred at 0° C. for 1 h, and then concentrated. To a suspension of the residue in THF (60.0 mL) at 0° C. was added MSA (8.52 mL, 53.1 mmol). Upon stirring, the mixture turned into a clear solution. To the solution at 0° C. was added DIPEA (7.95 mL, 45.5 mmol), followed by tert-butyl bromoacetate (2.69 mL, 18.20 mmol) dropwise. The mixture was stirred at 0° C. for 1 h, and then rt for 16 h. The mixture was diluted with EtOAc and a mixture of satd. aq. NH₄Cl (10 mL) and H₂O (20 mL). The pH was adjusted to pH 4 with 2M aq. H₂SO₄. The mixture was extracted twice with EtOAc. The combined organic extracts were washed with brine, dried over Na₂SO₄, filtered, and concentrated. The resulting residue was purified by silica gel flash column chromatography (gradient, CH₂Cl₂/MeOH=1/0 to 80/20) to afford the title compound (5.60 g, 10.76 mmol). ¹H NMR (500 MHz, DMSO-d₆) δ 12.58 (br s, 1H), 7.90 (d, J=7.5 Hz, 2H), 7.72 (d, J=7.5 Hz, 2H), 7.66 (d, J=8.5 Hz, 1H), 7.42 (dd, J=7.5, 7.5 Hz, 2H), 7.33 (dd, J=7.5, 7.5 Hz, 2H), 4.37-4.19 (m, 3H), 4.04-3.85 (m, 1H), 3.20-2.95 (m, 2H), 2.91-2.67 (m, 2H), 2.26-1.96 (m, 2H), 1.72-0.91 (m, 16H). MS (ESI+): m/z 509.4 [M+H].

### Example-1-9

The following compounds were synthesized as outlined for the preparation of Example-1-9-01 employing appropriate starting materials in the table.

| | Structure and IUPAC name |
|---|---|
| | Fmoc-protected starting material-A |
| | Starting material-B |
| Example | NMR and MS |
| 1-9-02 | 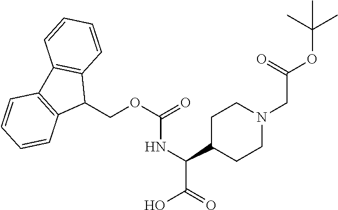 [SMILES: CC(C)(C)OC(=O)CN1CCC([C@H](NC(=O)OCC2c3ccccc3-c3ccccc32)C(=O)O)CC1] |
| | (2S)-2-{1-[2-(tert-butoxy)-2-oxoethyl]piperidin-4-yl}-2- |
| | ({[(9H-fluoren-9-yl)methoxy]carbonyl}amino)acetic acid |
| | (2S)-2-{1-[(tert-butoxy)carbonyl]piperidin-4-yl}-2-({[(9H- |
| | fluoren-9-yl)methoxy]carbonyl} amino)acetic acid |
| | (CAS: 204058-24-2) |
| | tert-butyl bromoacetate |
| | ¹H NMR (500 MHz, DMSO-d₆) δ 12.62 (br s, 1H), 7.89 (d, |
| | J = 7.5 Hz, 2H), 7.78-7.60 (m, 3H), 7.43-7.48 (m, 2H), 7.35- |
| | 7.30 (m, 2H), 4.30-4.20 (m, 3H), 3.92-3.86 (m, 1H), 3.06 (s, |
| | 2H), 2.87-2.80 (m, 2H), 2.05-2.16 (m, 2H), 1.72-1.62 (m, |
| | 1H), 1.57-1.47 (m, 2H), 1.43-1.28 (m, 11H). |
| | MS (ESI+): m/z 495.3 [M + H]. |
| 1-9-03 | 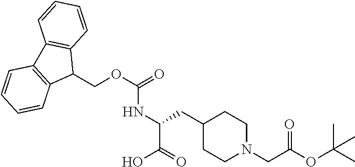 [SMILES: CC(C)(C)OC(=O)CN1CCC(C[C@@H](NC(=O)OCC2c3ccccc3-c3ccccc32)C(=O)O)CC1] |
| | (2R)-3-{1-[2-(tert-butoxy)-2-oxoethyl]piperidin-4-yl}-2- |
| | ({[(9H-fluoren-9-yl)methoxy]carbonyl}amino)propanoic acid |
| | (2R)-3-{1-[(tert-butoxy)carbonyl]piperidin-4-yl}-2-({[(9H- |
| | fluoren-9-yl)methoxy]carbonyl}amino)propanoic acid |
| | (CAS: 313052-00-5) tert-butyl bromoacetate |
| | Substantially identical to Example-1-9-01 |

### Example-1-10-01: (2S)-3-{4-[({4-[2-(tert-butoxy)-2-oxoethyl]piperazine-1-carbonyl}amino)methyl]phenyl}-2-({[(9H-fluoren-9-yl)methoxy]carbonyl}(methyl)amino)propanoic acid

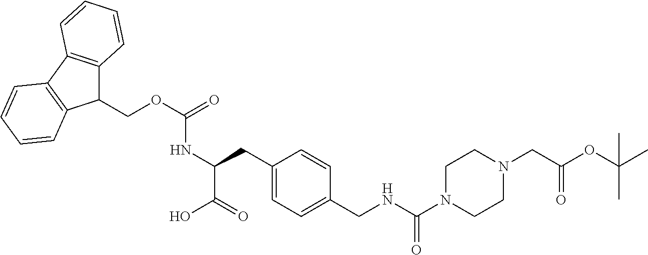 [SMILES: CC(C)(C)OC(=O)CN1CCN(C(=O)NCc2ccc(C[C@H](NC(=O)OCC3c4ccccc4-c4ccccc43)C(=O)O)cc2)CC1]

To a suspension of Zn (31.62 g, 486.49 mmol) in DMF at rt was added I₂ (12.36 g, 48.65 mmol). The mixture was stirred for 10 min. To the mixture at rt was added methyl (2R)-2-{[(9H-fluoren-9-ylmethoxy)carbonyl]amino}-3-iodopropanoate (CAS: 156017-42-4, 87.76 g, 194.59 mmol) The mixture was stirred at rt for 0.5 h. To the mixture were added 1-(4-bromophenyl)methanamine (30 g, 162.162 mmol), Pd₂(dba)₃ (7.43 g, 8.108 mmol), and SPhos (13.33 g, 32.43 mmol). The mixture was stirred at 50° C. for 2 h. The reaction mixture was filtered, and then the filtrate was diluted with EtOAc. The resulting mixture was washed three times with brine, and then dried over Na₂SO₄, filtered, and then concentrated. The resulting residue was triturated with petroleum ether/EtOAc (1/1), and then collected by filtration, which was dissolved in CH₂Cl₂ (460 mL). To the solution at 0° C. were added triphosgene (7.13 g, 24.04 mmol) and DIEA (27.92 mL, 160.28 mmol). The mixture was stirred at 0° C. for 15 min. To the mixture at 0° C. were added NaHCO₃ (17.95 g, 213.70 mmol) in H₂O (230 mL) and tert-butyl 2-(piperazin-1-yl) acetate (21.40 g, 106.85 mmol). The mixture was stirred at rt for 2 h, and then NaH₂PO₄ was added to the mixture. The mixture was extracted three times with CH₂Cl₂. The combined organic extracts were washed three times with brine, and then dried over Na₂SO₄, filtered, and then concentrated.

The resulting residue was purified by silica gel flash column chromatography (petroleum ether/EtOAc=50/50). Appropriate fractions were collected and concentrated (28 g). To a mixture of the obtained material in iPrOH (1200 mL) and H₂O (400 mL) at 0° C. were added LiOH·H₂O (7.66 g, 182.70 mmol) and CaCl₂ (81.10 g, 730.84 mmol). The mixture was stirred at rt for 16 h. The reaction was quenched with NaH₂PO₄.

The resulting mixture was extracted three times with EtOAc. The combined organic extracts were washed three times with brine, dried over Na₂SO₄, filtrated, and then concentrated. The resulting residue was purified by silica gel flash column chromatography (isocratic, petroleum ether/EtOAc=50/50 then CH₂Cl₂/MeOH=91/9) to afford the title compound (15.05 g, 23.42 mmol). ¹H NMR (400 MHz, DMSO-d₆) δ 12.70 (br s, 1H), 7.88 (d, J=7.6 Hz, 2H), 7.70-7.63 (m, 3H), 7.43-7.38 (m, 2H), 7.35-7.25 (m, 2H), 7.22-7.12 (m, 4H), 7.06-6.96 (m, 1H), 4.25-4.09 (m, 6H), 3.11 (s, 2H), 3.04 (dd, J=13.8, 4.4 Hz, 1H), 2.84 (dd, J=13.8, 10.4 Hz, 1H), 2.48-2.42 (m, 4H), 1.40 (s, 9H). MS (ESI+): m/z 643.2 [M+H].

### Example-1-10

The following compounds were synthesized as outlined for the preparation of Example-1-10-01 employing appropriate starting materials in the table.

| | Structure and IUPAC name |
|---|---|
| | Fmoc-protected starting material-A |
| | Starting material-B |
| Example | NMR and MS |
| 1-10-02 | 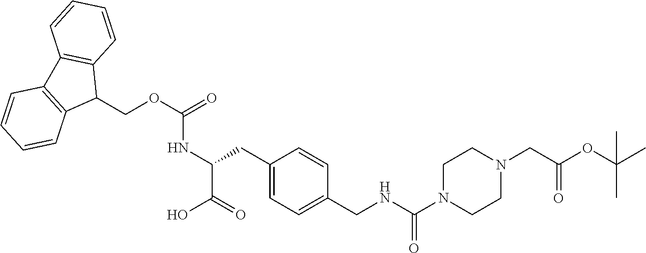 [SMILES: CC(C)(C)OC(=O)CN1CCN(C(=O)NCc2ccc(C[C@@H](NC(=O)OCC3c4ccccc4-c4ccccc43)C(=O)O)cc2)CC1] |
| | (2R)-3-{4-[({4-[2-(tert-butoxy)-2-oxoethyl]piperazine-1-carbonyl}amino) |
| | methyl]phenyl}-2-({[(9H-fluoren-9-yl)methoxy]carbonyl}amino)propanoic |
| | acid |
| | methyl (2S)-2-{[(9H-fluoren-9-ylmethoxy)carbonyl]amino}-3-iodopropanoate |
| | (CAS: 527696-77-1) |
| | 1-(4-bromophenyl)methanamine (CAS: 3959-07-7) |
| | Substantially identical to Example-1-10-01 |
| 1-10-03 | 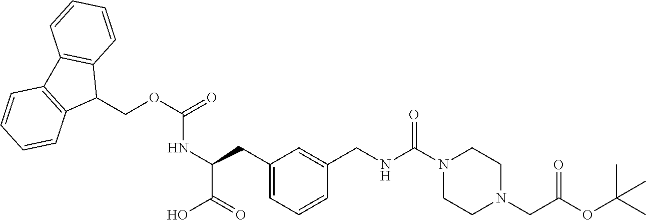 [SMILES: CC(C)(C)OC(=O)CN1CCN(C(=O)NCc2cccc(C[C@H](NC(=O)OCC3c4ccccc4-c4ccccc43)C(=O)O)c2)CC1] |
| | (2S)-3-{3-[({4-[2-(tert-butoxy)-2-oxoethyl]piperazine-1- |
| | carbonyl}amino)methyl]phenyl}-2-({[(9H-fluoren-9- |
| | yl)methoxy]carbonyl}amino)propanoic acid |
| | methyl (2R)-2-{[(9H-fluoren-9-ylmethoxy)carbonyl]amino}-3-iodopropanoate |
| | 1-(3-bromophenyl)methanamine (CAS: 10269-01-9) |
| | ¹H NMR (400 MHz, DMSO-d₆) δ 7.88 (d, J = 7.5 Hz, 2H), 7.66 (t, J = |
| | 6.6 Hz, 2H), 7.59-7.49 (m, 1H), 7.43-7.36 (m, 2H), 7.34-7.25 (m, |
| | 2H), 7.22-7.16 (m, 1H), 7.16-7.06 (m, 3H), 7.06-6.93 (m, 1H), 4.27- |
| | 4.08 (m, 6H), 3.33-3.28 (m, 4H), 3.10 (s, 2H), 3.08-3.03 (m, 1H), |
| | 2.91-2.82 (m, 1H), 2.47-2.40 (m, 4H), 1.40 (s, 9H). MS (ESI+): |
| | m/z 643.2 [M + H]. |

### Example-1-11-01: (2S)-3-(6-{[2-(tert-butoxy)-2-oxoethyl]amino}pyridin-3-yl)-2-({[(9H-fluoren-9-yl)methoxy]carbonyl}amino)propanoic acid

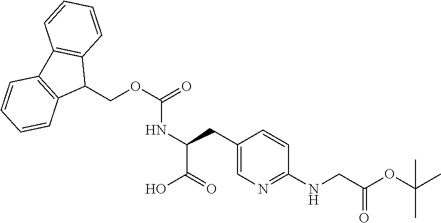 [SMILES: CC(C)(C)OC(=O)CNc1ccc(C[C@H](NC(=O)OCC2c3ccccc3-c3ccccc32)C(=O)O)cn1]

To a solution of 5-bromo-2-fluoropyridine (8.80 g, 50.0 mmol) and tert-butyl glycinate (7.21 g, 55.0 mmol) in DMSO (25 mL) at rt was added DIPEA (19.2 mL, 110 mmol). The mixture was stirred at 100° C. for 5 h, and then diluted with iPrOAc. The mixture was washed 3 times with H₂O, dried over Na₂SO₄, filtered, and then concentrated. The resulting residue was purified by silica gel flash column chromatography (gradient, hexane/EtOAc=100/0 to 0/100) to afford tert-butyl 2-[(5-bromopyridin-2-yl)amino]acetate (1.72 g, 6.00 mmol). To a suspension of Zn (1.18 g, 18.0 mmol) in DMF (12.0 mL) at rt was added iodine (1.52 g, 6.00 mmol). The mixture was stirred at rt for 30 min. To the mixture at rt was added methyl (2R)-2-({[(9H-fluoren-9-yl)methoxy]carbonyl}amino)-3-iodopropanoate (CAS: 156017-42-4, 3.25 g, 7.20 mmol). The mixture was stirred at rt for 1 h. A mixture of Pd₂(dba)₃ CHCl₃ adduct (0.31 g, 0.30 mmol) and SPhos (0.49 g, 1.20 mmol) in DMF (18.0 mL) was stirred at 60° C. for 2 h.

To the mixture at rt was added tert-butyl 2-[(5-bromopyridin-2-yl)amino]acetate (1.72 g, 6.00 mmol). The mixture was stirred at 50° C. for 20 min. To this mixture was added the solution of the organozinc reagent. The mixture was stirred at 50° C. for 2 h. The reaction was quenched by adding iPrOAc and H₂O at rt. The mixture was filtered. The filtrate was extracted with iPrOAc. The organic extract was washed with brine, dried over Na₂SO₄, filtered, and then concentrated. The resulting residue was purified by silica gel flash column chromatography (gradient, heptane/EtOAc=100/0 to 0/100). Appropriate fractions were concentrated. To the resulting residue (1.37 g, 2.58 mmol) in iPrOH (36.1 mL) at 0° C. were added CaCl₂ (4.58 g, 41.2 mmol) and a solution of LiOH (0.25 g, 10.3 mmol) in H₂O (15.5 mL). The mixture was stirred at 30° C. for 16 h. iPrOH was removed. The pH of the mixture was adjusted to 1 with 1M HCl. The mixture was extracted with EtOAc. The organic extract was washed with brine, dried over Na₂SO₄, filtered, and concentrated. The resulting residue was triturated with iPrOAc and heptane, and then collected by filtration to afford the title compound (0.671 g, 1.30 mmol). ¹H NMR (500 MHz, DMSO-d₆) δ 12.75 (br s, 1H), 7.89 (d, J=7.5 Hz, 2H), 7.84 (s, 1H), 7.73 (d, J=8.5 Hz, 1H), 7.66 (dd, J=11.0, 7.5 Hz, 2H), 7.48-7.22 (m, 5H), 7.10-6.31 (m, 2H), 4.29-4.02 (m, 4H), 3.91 (s, 2H), 2.99-2.83 (m, 1H), 2.76-2.56 (m, 1H), 1.38 (s, 9H). MS (ESI+): m/z 518.4 (M+H).

### Reference Example-1-2: methyl (2R)-2-({[(9H-fluoren-9-yl)methoxy]carbonyl}(methyl)amino)-3-iodopropanoate

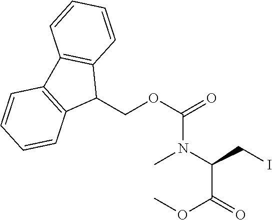 [SMILES: COC(=O)[C@H](CI)N(C)C(=O)OCC1c2ccccc2-c2ccccc21]

To a mixture of methyl (2S)-3-hydroxy-2-(methylamino)propanoate (CAS: 111934-24-8, 64.2 g, 482 mmol) in H₂O (700 mL) and 1,4-dioxane (700 mL) at 0° C. were added Na₂CO₃ (81.0 g, 964 mmol) and Fmoc-OSu (179 g, 531 mmol). The mixture was stirred at rt for 16 h. The reaction was quenched with H₂O. The mixture was extracted three times with EtOAc. The combined organic extracts were dried over Na₂SO₄, filtered, and then concentrated. The resulting residue was purified by silica gel flash column chromatography (petroleum ether/EtOAc=50/50). Appropriate fractions were concentrated (73.8 g, 208 mmol). To a solution of the obtained material (60.0 g, 169 mmol) in CH₂Cl₂ (1.00 L) at 0° C. were added triphenylphosphine (66.4 g, 253 mmol), imidazole (17.2 g, 253 mmol), and iodine (64.3 g, 253 mmol). The mixture was stirred at rt for 0.25 h and then concentrated. The resulting residue was purified by silica gel flash column chromatography (isocratic, petroleum ether/EtOAc=83/17) to afford the title compound (11.0 g, 23.6 mmol). MS (ESI+): m/z 466.2 [M+H].

### Example-1-12-01: (2R)-3-{3-[(tert-butoxy)carbonyl]phenyl}-2-({[(9H-fluoren-9-yl)methoxy]carbonyl}amino)propanoic acid

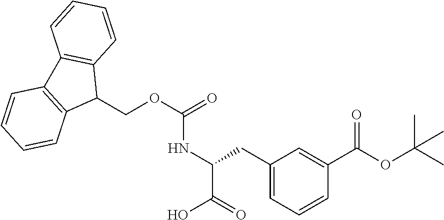 [SMILES: CC(C)(C)OC(=O)c1cccc(C[C@@H](NC(=O)OCC2c3ccccc3-c3ccccc32)C(=O)O)c1]

To a suspension of Zn (3.16 g, 48.4 mmol) in DMF (100 mL) was added I₂ (0.62 g, 2.43 mmol). The mixture was stirred at rt for 10 min. To the mixture at rt was added methyl (2S)-2-[[(9H-fluoren-9-ylmethoxy)carbonyl]amino]-3-iodopropanoate (CAS: 527696-77-1, 7.30 g, 16.2 mmol). The mixture was stirred at rt for an additional 1 h. To the mixture were added tert-butyl 3-bromobenzoate (CAS: 69038-74-0, 4.16 g, 16.2 mmol), Pd₂(dba)₃ CHCl₃ adduct (0.50 g, 0.485 mmol), and SPhos (0.33 g, 0.81 mmol). The mixture was stirred at 50° C. for an additional 12 h, and then filtered. The filtrate was diluted with H₂O. The mixture was extracted three times with EtOAc.

The combined organic extracts were washed with brine, dried over Na₂SO₄, filtered, and then concentrated. The resulting residue was purified by silica gel flash column chromatography (gradient, petroleum ether/EtOAc=99/1 to 75/25). Appropriate fractions were concentrated. To a solution of the resulting residue (5.36 g, 10.7 mmol) in ClCH₂CH₂Cl (50 mL) was added trimethyltin hydroxide (3.89 g, 21.5 mmol). The resulting mixture was stirred at 60° C. for 3 h. The reaction was quenched with aq. citric acid. The mixture was extracted three times with CH₂Cl₂. The combined organic extracts were washed with brine, dried over Na₂SO₄, filtered, and then concentrated. The resulting residue was purified by silica gel flash column chromatography (gradient, petroleum ether/EtOAc=99/1 to 50/50), and then further purified by reversed phase silica gel flash column chromatography (C18 silica gel, gradient, H₂O/CH₃CN=50/50 to 20/80) to afford the title compound (3.20 g, 6.56 mmol). ¹H NMR (400 MHz, Methanol-d₄) δ 7.93-7.76 (m, 4H), 7.61-7.44 (m, 3H), 7.42-7.24 (m, 5H), 4.48 (dd, J=9.6, 4.8 Hz, 1H), 4.31 (dd, J=10.0, 6.8 Hz, 1H), 4.24-4.12 (m, 2H), 3.29 (d, J=4.8 Hz, 1H), 3.04 (dd, J=13.9, 9.7 Hz, 1H), 1.56 (s, 9H); MS (ESI+): m/z 486.1 [M+H]

### Example-1-12-02: (2S)-3-{1-[(tert-butoxy)carbonyl]-1H-pyrrolo[3,2-c]pyridin-3-yl}-2-({[(9H-fluoren-9-yl)methoxy]carbonyl}amino)propanoic acid

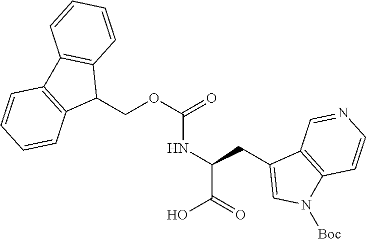 [SMILES: CC(C)(C)OC(=O)n1cc(C[C@H](NC(=O)OCC2c3ccccc3-c3ccccc32)C(=O)O)c2cnccc21]

To a suspension of Zn (18.5 g, 283 mmol) in DMF (800 mL) at rt was added I₂ (3.6 g, 14.18 mmol), and then the mixture was stirred for 10 min. To the mixture at rt was added methyl (2R)-2-{[(9H-fluoren-9-ylmethoxy)carbonyl]amino}-3-iodopropanoate (CAS: 156017-42-4, 64.0 g, 142 mmol). The mixture was stirred at rt for an additional 1 h. To the mixture was added Pd₂(dba)₃ (2.17 g, 2.37 mmol), SPhos (1.94 g, 4.73 mmol), and tert-butyl 3-bromopyrrolo[3,2-c]pyridine-1-carboxylate (CAS: 192189-16-5, 28.0 g, 94.0 mmol). The mixture was stirred at 60° C. for 3 h, and then filtered. The filtrate was diluted with EtOAc. The mixture was washed three times with H₂O, dried over Na₂SO₄, filtered, and then concentrated. The resulting residue was purified by silica gel flash column chromatography (petroleum ether/EtOAc=63/37). Appropriate fractions were concentrated (31.0 g, 57.2 mmol). To a mixture of the obtained material (21.0 g, 38.8 mmol) in 2-propanol (600 mL) and H₂O (200 mL) at 0° C. were added CaCl₂ (69.0 g, 622 mmol) and LiOH·H₂O (6.50 g, 155 mmol). The mixture was stirred at rt for 1 h. The pH of the mixture was adjusted to pH 7 with aq. citric acid. The resulting mixture was extracted three times with EtOAc. The combined organic extracts were washed three times with H₂O, dried over Na₂SO₄, filtered, and then concentrated. The resulting residue was triturated with Et₂O. The obtained solid sample was purified by reversed phase silica gel flash column chromatography (C18 silica gel, gradient, H₂O/CH₃CN=100/0 to 0/100) to afford the title compound (10.0 g, 19.0 mmol). ¹H NMR (500 MHz, DMSO-d₆) δ 12.90 (br s, 1H), 8.99 (s, 1H), 8.45 (d, J=5.5 Hz, 1H), 7.91-7.86 (m, 4H), 7.62-7.56 (m, 3H), 7.41-7.37 (m, 2H), 7.27-7.21 (m, 2H), 4.39-4.34 (m, 1H), 4.20-4.14 (m, 3H), 3.27 (dd, J=15.0, 4.5 Hz, 1H), 3.12 (dd, J=15.0, 10.5 Hz, 1H), 1.57 (s, 9H); MS (ESI+): m/z 528 [M+H]

### Example-1-12

The following compounds were synthesized as outlined for the preparation of Example-1-12-01 or Example-1-12-02 employing appropriate starting materials in the table.

| | Structurer and IUPAC name |
|---|---|
| | Fmoc-protected starting material-A |
| | Starting material-B |
| Example | NMR and MS |
| 1-12-03 | 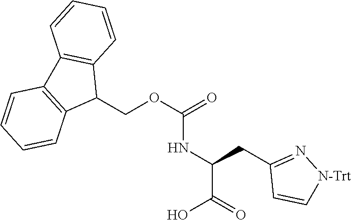 |
| | (2S)-2-({[(9H-fluoren-9-yl)methoxy]carbonyl}amino)-3-[1- |
| | (triphenyl-methyl)-1H-pyrazol-3-yl]propanoic acid |
| | methyl (2R)-2-({[(9H-fluoren-9-yl)methoxy]carbonyl}amino)- |
| | 3-iodo-propanoate (CAS: 156017-42-4) |
| | 3-iodo-1-(triphenylmethyl)-1H-pyrazole (CAS: 1802434-33-8) |
| | ¹H NMR (400 MHz, DMSO-d₆) δ 12.74 (br s, 1H), 7.89 (d, J = |
| | 7.5 Hz, 2 H), 7.67-7.65 (m, 2 H), 7.57 (d, J = 8.5 Hz, 1 H), |
| | 7.40 (dd, J = 7.5, 7.5 Hz, 2 H), 7.31-7.24 (m, 11 H), 7.15 |
| | (s, 1H), 7.03-7.02 (m, 6 H), 6.16 (s, 1 H), 4.29-4.12 (m, |
| | 4 H), 3.03 (dd, J = 14.5, 4.5 Hz, 1 H), 2.90 (dd, J = |
| | 14.5, 9.5 Hz, 1 H); MS (ESI+): m/z 620.5 [M + H]. |
| 1-12-04 | 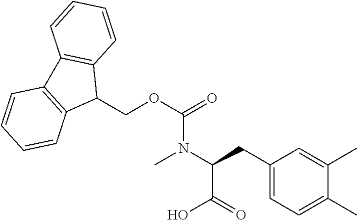 [SMILES: Cc1ccc(C[C@@H](C(=O)O)N(C)C(=O)OCC2c3ccccc3-c3ccccc32)cc1C] |
| | (2S)-3-(3,4-dimethylphenyl)-2-({[(9H-fluoren-9-yl)methoxy] |
| | carbonyl}(methyl)amino)propanoic acid |
| | Reference example-1-2 |
| | 4-bromo-1,2-dimethylbenzene (CAS: 583-71-1) |
| | ¹H NMR (500 MHz, DMSO-d₆) δ 12.90 (br s, 1 H), 7.89 (d, |
| | J = 7.5 Hz, 2 H), 7.58-7.39 (m, 4 H), 7.33-7.24 (m, 3 H), |
| | 7.18-7.13 (m, 2 H), 7.03-6.76 (m, 3 H), 4.83-4.61 |
| | (m, 1 H), 4.30-4.10 (m, 3 H), 3.26- |
| | 2.76 (m, 2 H), 2.72-2.67 (m, 3 H), 2.20-2.10 (m, 6 H); |
| | MS (ESI+): m/z 429.9 [M + H] |
| 1-12-05 | 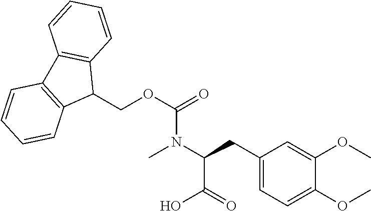 [SMILES: COc1ccc(C[C@@H](C(=O)O)N(C)C(=O)OCC2c3ccccc3-c3ccccc32)cc1OC] |
| | (2S)-3-(3,4-dimethoxyphenyl)-2-({[(9H-fluoren-9-yl)methoxy] |
| | carbonyl}(methyl)amino)propanoic acid |
| | Reference example-1-2 |
| | 4-bromo-1,2-dimethoxybenzene (CAS: 2859-78-1) |
| | ¹H NMR (500 MHz, DMSO-d₆) δ 12.89 (br s, 1 H), 7.88 (dd, |
| | J = 7.5, 2.0 Hz, 2 H), 7.54 (dd, J = 16.0 Hz, 7.0 Hz, 1 H), 7.47- |
| | 7.38 (m, 3 H), 7.33-7.26 (m, 2 H), 6.86-6.78 (m, 2 H), 6.73- |
| | 6.57 (m, 1 H), 4.86-4.74 (m, 1 H), 4.27-4.11 (m, 3 H), 3.71- |
| | 3.63 (m, 6 H), 3.18-3.06 (m, 1 H), 3.00-2.82 (m, 1 H), |
| | 2.71 (s, 3 H); MS (ESI+): |
| | m/z 462.3 [M + H] |
| 1-12-06 | 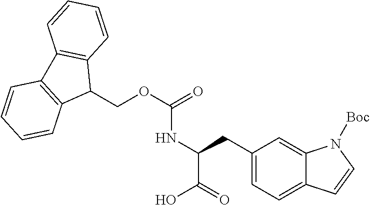 [SMILES: CC(C)(C)OC(=O)n1ccc2ccc(C[C@H](NC(=O)OCC3c4ccccc4-c4ccccc43)C(=O)O)cc21] |
| | (2S)-3-{1-[(tert-butoxy)carbonyl]-1H-indol-6-yl}-2-({[(9H- |
| | fluoren-9-yl)methoxy]carbonyl}amino)propanoic acid |
| | methyl (2R)-2-({[(9H-fluoren-9-yl)methoxy]carbonyl}amino)- |
| | 3-iodo-propanoate (CAS: 156017-42-4) |
| | tert-butyl 6-bromo-1H-indole-1-carboxylate (CAS: 147621-26-9) |
| | ¹H NMR (500 MHz, DMSO-d₆) δ 12.79 (br s, 1 H), 8.02 (s, |
| | 1 H), 7.87 (d, J = 7.5 Hz, 2 H), 7.76 (d, J = 7.5 Hz, 1 H), |
| | 7.64-7.49 (m, 4 H), 7.44-7.33 (m, 2 H), 7.26 (dd, J = 7.5, |
| | 7.5 Hz, 1 H), 7.19 (dd, J = 9.5, 8.5 Hz, 2 H), 6.70-6.64 (m, |
| | 1 H), 4.29-4.06 (m, 4 H), 3.21 (dd, J = 14.0, 4.5 Hz, 1 H), |
| | 3.03-2.89 (m, 1 H), 1.59 (s, 9 H); MS |
| | (ESI+): m/z 527.4 [M + H] |

### Example-1-13: (2S)-3-{1-[(tert-butoxy)carbonyl]-5-methyl-1H-imidazol-4-yl}-2-({[(9H-fluoren-9-yl)methoxy]carbonyl}amino)propanoic acid

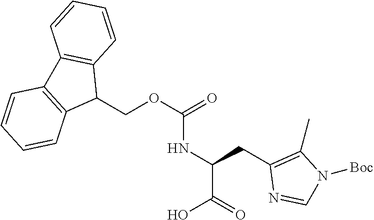 [SMILES: Cc1c(C[C@H](NC(=O)OCC2c3ccccc3-c3ccccc32)C(=O)O)ncn1C(=O)OC(C)(C)C]

To a solution of 4-bromo-5-methyl-1H-imidazole (CAS: 15813-08-8, 12.9 g, 80.00 mmol) in CH₂Cl₂ (180 mL) at 4° C. were added DMAP (1.95 g, 15.96 mmol), and DIPEA (20.64 g, 159.70 mmol), followed by Boc₂O (20.95 g, 96.00 mmol). The mixture was stirred at rt for 1.5 h, and then concentrated. The resulting residue was diluted with EtOAc, and then washed twice with brine, dried over Na₂SO₄, filtered, and then concentrated. The resulting residue was purified by silica gel flash column chromatography (gradient, petroleum ether/EtOAc=100/0 to 85/15) to afford the bromoimidazole derivative-1 (13.5 g, 51.7 mmol). To a suspension of Zn (9.81 g, 150 mmol) in DMF (240 mL) was added I₂ (950 mg, 3.74 mmol), and then the mixture was stirred at rt for 5 min. To the mixture was added methyl (2R)-2-[[(9H-fluoren-9-ylmethoxy)carbonyl]amino]-3-iodopropanoate (CAS: 156017-42-4, 27.1 g, 60.0 mmol), I₂ (950 mg, 3.74 mmol), and then the mixture was stirred at rt for another 0.75 h. To the mixture at rt were added Pd₂(dba)₃ CHCl₃ adduct (1.55 g, 1.50 mmol), SPhos (1.03 g, 2.51 mmol), and the bromoimidazole derivative-1 (13.1 g, 50.0 mmol) in DMF (60.0 mL).

The mixture was stirred at 50° C. for 3 h, and then filtered, and then diluted with EtOAc. The mixture was washed three times with brine, dried over Na₂SO₄, filtered, and then concentrated. The resulting residue was purified by silica gel flash column chromatography (gradient, CH₂Cl₂/EtOAc=100/0 to 85/15). Appropriate fractions were concentrated (8.60 g, 17.0 mmol). To a solution of obtained compound in ClCH₂CH₂Cl (60 mL) at rt was added trimethyltin hydroxide (8.64 g, 47.78 mmol). The mixture was stirred at 50° C. for 4 h, and then diluted with EtOAc. The organic layer was washed successively twice with 10% aq. citric acid (120 mL), twice with brine, dried over Na₂SO₄, filtered, and then concentrated. The resulting residue was purified by silica gel flash column chromatography (gradient, petroleum ether/EtOAc=5/95 to 40/60) to afford the title compound (3.94 g, 8.02 mmol). ¹H NMR (500 MHz, DMSO-d₆) δ 12.66 (br s, 1H), 8.04 (s, 1H), 7.89 (d, J=7.5 Hz, 2H), 7.65 (d, J=7.5 Hz, 2H), 7.53 (d, J=8.5 Hz, 1H), 7.41 (dd, J=7.5, 7.0 Hz, 2H), 7.32-7.28 (m, 2H), 4.30-4.15 (m, 4H), 2.89-2.79 (m, 2H), 2.25 (s, 3H), 1.53 (s, 9H); MS (ESI+): m/z 493.2 [M+H]

### Example-1-14: (2S)-2-({[(9H-fluoren-9-yl)methoxy]carbonyl}amino)-3-(2-oxo-1,2-dihydropyridin-4-yl)propanoic acid

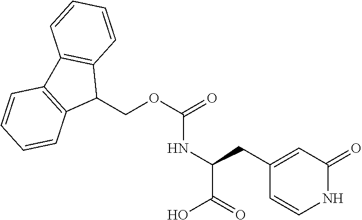 [SMILES: O=C(N[C@@H](Cc1cc[nH]c(=O)c1)C(=O)O)OCC1c2ccccc2-c2ccccc21]

To a solution of 4-bromopyridin-2-ol (50.0 g, 287 mmol) in DMF (600 mL) at 0° C. was added sodium hydride (13.8 g, 575 mmol), followed by chloromethyl methyl ether (CAS: 107-30-2, 55.5 g, 690 mmol). The mixture was stirred at 0° C. for 2 h, and then rt for ca. 16 h. The reaction was quenched with H₂O. The mixture was extracted three times with EtOAc (400 mL). The combined organic extracts were washed twice with brine, dried over Na₂SO₄, filtered, and then concentrated. The resulting residue was purified by silica gel flash column chromatography (gradient, petroleum ether/EtOAc=100/0 to 50/50) to afford the corresponding MOM derivative-1 (38.0 g, 174 mmol). To a suspension of Zn (9.00 g, 138 mmol) in DMF (60.0 mL) was added I₂ (1.75 g, 6.88 mmol). The resulting mixture was stirred at rt for 10 min. To this was added methyl (2R)-2-[[(9H-fluoren-9-ylmethoxy)carbonyl]amino]-3-iodopropanoate (24.8 g, 55.0 mmol). The resulting mixture was stirred at rt for an additional 0.5 h, and then were added the corresponding MOM derivative-1 (10.0 g, 45.9 mmol), SPhos (941 mg, 2.29 mmol), Pd₂(dba)₃ CHCl₃ adduct (1.42 g, 1.38 mmol). The mixture was stirred at 50° C. for an additional 3 h. The reaction was then quenched with H₂O. The resulting mixture was extracted three times with EtOAc. The combined organic extracts were washed twice with brine, dried over Na₂SO₄, filtered, and then concentrated. The resulting residue was purified by silica gel flash column chromatography (gradient, petroleum ether/EtOAc=99/1 to 50/50). Appropriate fractions were concentrated. To the resulting residue (12.0 g, 26.0 mmol) in 1,4-dioxane (120 mL) was added conc. HCl (120 mL). The mixture was stirred for 1 h at 100° C. and then concentrated. The resulting residue was triturated with H₂O/EtOAc=1/3 to afford the title compound (5.79 g, 14.3 mmol). ¹H NMR (500 MHz, DMSO-d₆) δ 11.38 (br s, 1H), 7.88 (d, J=7.5 Hz, 2H), 7.65 (dd, J=7.0, 6.5 Hz, 2H), 7.45-7.37 (m, 2H), 7.35-7.28 (m, 2H), 7.23 (br s, 1H), 6.17 (br s, 1H), 6.07 (br s, 1H), 4.26-3.95 (m, 4H), 2.93-2.79 (m, 1H), 2.71-2.56 (m, 1H); MS (ESI+): m/z 405.5 [M+H].

### Example-1-15: (2R)-3-(6-{[(tert-butoxy)carbonyl]amino}pyridin-3-yl)-2-({[(9H-fluoren-9-yl)methoxy]carbonyl}amino)propanoic acid

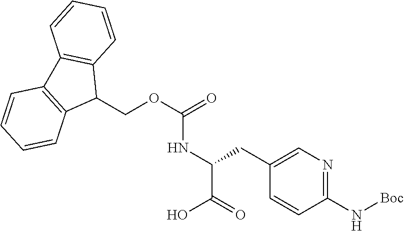 [SMILES: CC(C)(C)OC(=O)Nc1ccc(C[C@@H](NC(=O)OCC2c3ccccc3-c3ccccc32)C(=O)O)cn1]

To a suspension of Zn (239 mg, 3.66 mmol) in DMF (10 mL) at rt was added I₂ (92.8 mg, 0.366 mmol). The mixture was stirred for 10 min. To the mixture at rt was added methyl (2S)-2-{[(9H-fluoren-9-ylmethoxy)carbonyl]amino}-3-iodopropanoate (CAS: 527696-77-1, 550 mg, 1.22 mmol) and I₂ (92.8 mg, 0.366 mmol). The mixture was stirred at rt for 1 h. To the mixture at rt were added 4-bromopyridin-2-amine (CAS: 1072-97-5, 253 mg, 1.46 mmol), Pd₂(dba)₃ CHCl₃ adduct (63.1 mg, 0.06 mmol), and SPhos (50.0 mg, 0.12 mmol). The mixture was stirred at 50° C. for 2.5 h, and then filtered. The filtrate was extracted with EtOAc. The organic extract was washed three times with brine, dried over Na₂SO₄, filtered, and then concentrated. The resulting residue was purified by silica gel flash column chromatography (gradient, petroleum ether/EtOAc=91/9 to 50/50). Appropriate fractions were concentrated. To a solution of the resulting residue (380 mg, 0.91 mmol) in tert-BuOH (10 mL) at rt were added NaI (164 mg, 1.09 mmol) and Boc₂O (238 mg, 1.09 mmol). The mixture was stirred at rt for 16 h. The reaction was then quenched with H₂O. The mixture was extracted three times with EtOAc. The combined organic extracts were washed three times with brine, dried over Na₂SO₄, filtered, and then concentrated. The resulting residue was purified by silica gel flash column chromatography (gradient, petroleum ether/EtOAc=91/9 to 50/50). Appropriate fractions were concentrated. To a solution of the resulting residue (400 mg, 0.773 mmol) in 2-propanol (3 mL) and H₂O (1 mL) at 0° C. were added CaCl₂ (858 mg, 7.73 mmol) and LiOH (74.0 mg, 3.09 mmol). The mixture was stirred for at rt 16 h. The pH of the mixture was adjusted to pH 5 with aq. citric acid. The mixture was then filtered. The filtrate was extracted three times with EtOAc. The combined organic extracts were washed twice with brine, dried over Na₂SO₄, filtered, and then concentrated. The resulting residue was purified by silica gel flash column chromatography (gradient, CH₂Cl₂/MeOH=98/2 to 91/9) to afford the title compound (229 mg, 0.46 mmol). ¹H NMR (300 MHz, DMSO-d₆) δ 12.86 (br s, 1H), 9.68 (s, 1H), 8.12 (d, J=2.1 Hz, 1H), 7.88 (d, J=7.5 Hz, 2H), 7.78 (d, J=8.5 Hz, 1H), 7.71 (d, J=8.6 Hz, 1H), 7.66-7.59 (m, 3H), 7.43-7.36 (m, 2H), 7.36-7.23 (m, 2H), 4.23-4.12 (m, 4H), 3.09 (dd, J=13.7, 4.4 Hz, 1H), 2.88 (dd, J=13.8, 10.6 Hz, 1H), 1.45 (s, 9H); MS (ESI+): m/z 504.1 [M+H].

### Example-1-16: (2S)-2-({[(9H-fluoren-9-yl)methoxy]carbonyl}amino)-3-(6-hydroxy-1H-indol-3-yl)propanoic acid

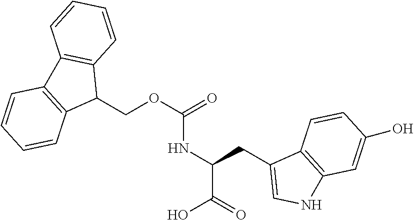 [SMILES: O=C(N[C@@H](Cc1c[nH]c2cc(O)ccc12)C(=O)O)OCC1c2ccccc2-c2ccccc21]

To a suspension of Zn (9.63 g, 147 mmol) in DMF (400 mL) was added I₂ (1.88 g, 7.41 mmol) at rt. The resulting solution was stirred for 10 min at rt. To the mixture at rt was added methyl (2R)-2-[[(9H-fluoren-9-ylmethoxy)carbonyl]amino]-3-iodopropanoate (22.3 g, 49.4 mmol), and the mixture was stirred for an additional 0.5 h. To the mixture at rt was added tert-butyl 6-(benzyloxy)-3-iodoindole-1-carboxylate (CAS: 914349-30-7, 18.5 g, 41.2 mmol). To the mixture was added Pd₂(dba)₃ CHCl₃ adduct (1.53 g, 1.48 mmol), SPhos (1.02 g, 2.49 mmol). The mixture was stirred at 50° C. for an additional 2 h, and then filtered, and then diluted with H₂O. The mixture was extracted three times with EtOAc. The combined organic extracts were washed with brine, dried over Na₂SO₄, filtered, and then concentrated. The resulting residue was purified by silica gel flash column chromatography (gradient, petroleum ether/EtOAc=75/25). Appropriate fractions were concentrated. (15.0 g, 23.2 mml). To a flask charged with Pd/C (3.00 g) was added a solution of the obtained material (15.0 g, 23.2 mmol) in EtOAc (150 mL). The mixture was stirred under H₂ atmosphere at rt for 3 h. The catalyst was removed by filtration. The filtrate was concentrated. The resulting residue was purified by silica gel flash column chromatography (gradient, petroleum ether/EtOAc=67/33). Appropriate fractions were concentrated. To a solution of the resulting residue (9.00 g, 19.7 mmol) in 1,4-dioxane (60 mL) was added conc. HCl (60 mL). The mixture was stirred at 100° C. for 4 h. The reaction mixture was cooled to room temperature, and then extracted three times with EtOAc. The combined organic extracts were washed with brine, dried over Na₂SO₄, filtered, and then concentrated. The resulting residue was purified by reversed phase silica gel flash column chromatography (C18 silica gel, gradient, CH₃CN/H₂O with 0.1% formic acid=35/65 to 75/25) to afford the title compound (5.21 g, 11.8 mmol). ¹H NMR (500 MHz, DMSO-d₆) δ 12.67 (br s, 1H), 10.44 (s, 1H), 8.86 (s, 1H), 7.88 (d, J=7.5 Hz, 2H), 7.69-7.65 (m, 3H), 7.43-7.39 (m, 2H), 7.33-7.27 (m, 3H), 6.93 (d, J=1.5 Hz, 1H), 6.69 (d, J=2.0 Hz, 1H), 6.52 (dd, J=8.5, 2.0 Hz, 1H), 4.23-4.15 (m, 4H), 3.18-3.08 (m, 1H), 2.94 (dd, J=14.5, 10.0 Hz, 1H); MS (ESI+): m/z 443.2 [M+H].

### Example-1-17: (2S)-3-(2H-1,3-benzodioxol-5-yl)-2-({[(9H-fluoren-9-yl)methoxy]carbonyl}(methyl)amino)propanoic acid

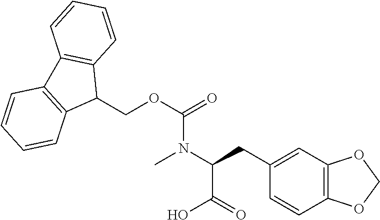 [SMILES: CN(C(=O)OCC1c2ccccc2-c2ccccc21)[C@@H](Cc1ccc2c(c1)OCO2)C(=O)O]

To a solution of (2S)-3-(2H-1,3-benzodioxol-5-yl)-2-[(tert-butoxycarbonyl)amino] propanoic acid (CAS: 209525-79-1, 32.0 g, 103 mmol) in THF (300 mL) at 0° C. was added sodium hydride (12.4 g, 60% dispersion in mineral oil) portionwise, followed by iodomethane (44.1 g, 310 mmol) dropwise. The mixture was stirred at rt for 24 h.

The reaction was quenched with H₂O. The pH of the mixture was adjusted to 5 with citric acid. The resulting mixture was extracted three times with EtOAc. The combined extracts were washed three times with brine, dried over Na₂SO₄, filtered, and then concentrated. The resulting residue was purified by silica gel flash column chromatography (gradient, petroleum ether/EtOAc=50/50). Appropriate fractions were concentrated. The resulting residue (27.0 g, 83.5 mmol) and 2M HCl in 1,4-dioxane (270 mL) was stirred at rt for 1 h. The resulting mixture was concentrated. To the resulting residue in (13.4 g, 51.6 mmol) in 1,4-dioxane (300 mL) and H₂O (100 mL) at 0° C. was added NaHCO₃ (8.67 g, 103 mmol), followed by Fmoc-OSu (17.4 g, 51.6 mmol) portionwise. The mixture was stirred at rt for 8 h. The pH of the solution was adjusted to 5 with citric acid. The mixture was extracted three times with EtOAc. The combined extracts were washed three times with brine, dried over Na₂SO₄, filtered, and then concentrated. The resulting residue was purified by reversed phase silica gel flash column chromatography (C18 silica gel, gradient, H₂O/CH₃CN with 0.1% NH₄HCO₃=60/40 to 30/70) to afford the title compound (5.01 g, 11.3 mmol). ¹H NMR (500 MHz, DMSO-d₆) δ 7.88 (dd, J=7.0, 6.0 Hz, 2H), 7.55 (d, J=7.5 Hz, 1H), 7.47 (dd, J=14.5, 7.5 Hz, 1H), 7.40 (dd, J=7.5, 7.0 Hz, 2H), 7.32-7.26 (m, 2H), 6.76 (dd, J=16.5, 8.0 Hz, 2H), 6.66-6.59 (m, 1H), 5.92-5.83 (m, 2H), 4.64-4.54 (m, 1H), 4.25-4.04 (m, 3H), 3.19-3.13 (m, 1H), 2.84-2.73 (m, 4H); MS (ESI+): m/z 446.2 [M+H].

### Example-1-18-01: (2S)-3-(4-ethylphenyl)-2-({[(9H-fluoren-9-yl)methoxy]carbonyl}(methyl)amino)propanoic acid

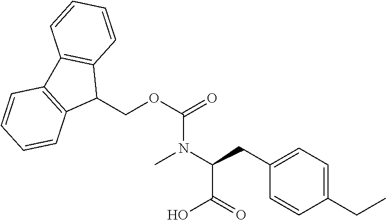 [SMILES: CCc1ccc(C[C@@H](C(=O)O)N(C)C(=O)OCC2c3ccccc3-c3ccccc32)cc1]

To a solution of (S)-2-((((9H-fluoren-9-yl)methoxy)carbonyl)amino)-3-(4-ethylphenyl)propanoic acid (CAS: 204384-72-5, 2.50 g, 6.02 mmol) in acetic acid (75 mL, 1309 mmol) at rt were added CSA (0.17 g, 0.72 mmol) and paraformaldehyde (0.752 g, 25.0 mmol). The mixture was stirred at 85° C. for 8 h, and then concentrated. The resulting residue was diluted with EtOAc and satd. aq NaHCO₃. The phases were separated, and the aqueous layer was extracted twice with EtOAc. The combined organic extracts were washed with brine, dried over MgSO₄, filtered, and then concentrated. The resulting residue was purified by silica gel flash column chromatography (gradient, hexane/EtOAc=1/0 to 2/1). Appropriate fractions were concentrated. To the resulting residue (2.16 g, 5.05 mmol) in CH₂Cl₂ (42.1 mL) at 0° C. were added triethylsilane (3.15 ml, 19.7 mmol) and 2,2,2-trifluoroacetic acid (31.3 mL, 409 mmol). The mixture was stirred at rt for 4 days and then concentrated. The resulting residue was purified by silica gel flash column chromatography (gradient, CH₂Cl₂/MeOH=100/0 to 90/10) to afford the title compound (1.87 g, 4.36 mmol). ¹H NMR (500 MHz, DMSO-d₆) δ 12.91 (br s, 1H), 7.96-7.80 (m, 2H), 7.74-7.23 (m, 6H), 7.16-6.92 (m, 4H), 4.88-4.42 (m, 1H), 4.41-4.10 (m, 3H), 3.21-2.93 (m, 2H), 2.73-2.65 (m, 2H), 2.62-2.52 (m, 2H), 1.20-1.04 (m, 3H); MS (ESI+): m/z 430.8 [M+H].

### Example-1-18

The following compounds were synthesized as outlined for the preparation of Example-1-18-01 employing appropriate starting materials in the table.

| | Structurer and IUPAC name |
|---|---|
| Ex- | Fmoc-protected starting material-A |
| ample | NMR and MS |
| 1-18-02 | 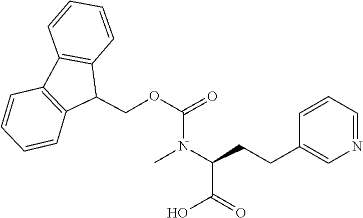 [SMILES: CN(C(=O)OCC1c2ccccc2-c2ccccc21)[C@@H](CCc1cccnc1)C(=O)O] |
| | (2S)-2-({[(9H-fluoren-9-yl)methoxy]carbonyl}(methyl) |
| | amino)-4-(pyridin-3-yl)butanoic acid |
| | (2S)-2-({[(9H-fluoren-9-yl)methoxy]carbonyl}amino)-4- |
| | (pyridin-3-yl)butanoic acid (CAS: 1260592-33-3) |
| | ¹H NMR (500 MHz, DMSO-d₆) δ 13.40-12.40 (m, 1 H), 8.51- |
| | 8.41 (m, 2 H), 7.92-7.86 (m, 3 H), 7.69-7.38 (m, 7 H), 4.49-4.17 |
| | (m, 4 H), 2.77-2.76 (m, 3 H), 2.55-2.44 (m, 2 H), 2.14-1.91 |
| | (m, 2 H); MS (ESI+): m/z 417.4 [M + H]. |
| 1-18-03 | 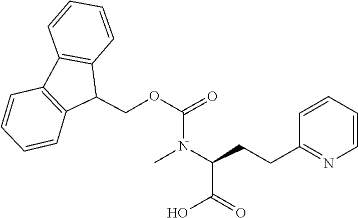 [SMILES: CN(C(=O)OCC1c2ccccc2-c2ccccc21)[C@@H](CCc1ccccn1)C(=O)O] |
| | (2S)-2-({[(9H-fluoren-9-yl)methoxy]carbonyl}(methyl) |
| | amino)-4-(pyridin-2-yl)butanoic acid |
| | (2S)-2-({[(9H-fluoren-9-yl)methoxy]carbonyl}amino)-4- |
| | (pyridin-2-yl)butanoic acid (CAS: 1260601-08-8) |
| | ¹H NMR (500 MHz, DMSO-d₆) δ 13.12-12.60 (m, 1 H), |
| | 8.48 (br s, 1 H), 7.92-7.86 (m, 2 H), 7.73- |
| | 7.52 (m, 3 H), 7.45-7.15 (m, 6 H), 4.53-4.45 (m, |
| | 1 H), 4.38-4.15 (m, 3 H), 2.76 (s, 3 H), 2.65-2.59 |
| | (m, 2 H), 2.31-2.27 (m, 1 H), 2.09-1.91 (m, 1 H); MS |
| | (ESI+): m/z 417.4 [M + H]. |
| 1-18-04 | 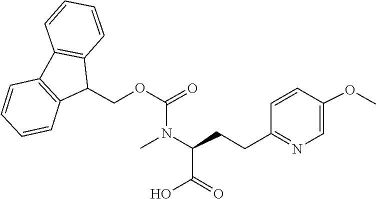 [SMILES: COc1ccc(CC[C@@H](C(=O)O)N(C)C(=O)OCC2c3ccccc3-c3ccccc32)nc1] |
| | (2S)-2-({[(9H-fluoren-9-yl)methoxy]carbonyl}(methyl)amino)- |
| | 4-(5-methoxypyridin-2-yl)butanoic acid |
| | (2S)-2-({[(9H-fluoren-9-yl)methoxy]carbonyl}amino)-4- |
| | (5-methoxy-pyridin-2-yl)butanoic acid (CAS: 2350064-93-4) |
| | ¹H NMR (500 MHz, DMSO-d₆) δ 13.01-12.69 (m, 1 H), |
| | 8.19 (d, J = 2.9 Hz, 1H), 7.92-7.86 (m, 2H), |
| | 7.67 (dd, J = 4.3, 7.3 Hz, 1H), 7.64-7.52 (m, |
| | 1H), 7.44-7.25 (m, 5H), 7.16-7.12 (m, 1H), 4.51-4.42 |
| | (m, 1H), 4.40-4.09 (m, 3H), 3.84-3.67 (m, 1H), |
| | 2.77 (s, 3H), 2.61-2.52 (m, 2H), 2.31-2.19 |
| | (m, 1H), 2.10-1.96 (m, 1H); MS (ESI+): |
| | m/z 447.3 [M + H]. |
| 1-18-05 | 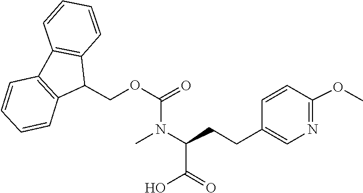 [SMILES: COc1ccc(CC[C@@H](C(=O)O)N(C)C(=O)OCC2c3ccccc3-c3ccccc32)cn1] |
| | (2S)-2-({[(9H-fluoren-9-yl)methoxy]carbonyl}(methyl)amino)- |
| | 4-(6-methoxypyridin-3-yl)butanoic acid |
| | (2S)-2-({[(9H-fluoren-9-yl)methoxy]carbonyl}amino)-4-(6- |
| | methoxypyridin-3-yl)butanoic acid (CAS: 2349871-82-3) |
| | ¹H NMR (500 MHz, DMSO-d₆) δ 12.96-12.76 (m, 1H), 7.96 |
| | (s, 1H), 7.94-7.86 (m, 2 H), 7.69-7.48 (m, 3 H), 7.44- |
| | 7.25 (m, 4 H), 6.75 (d, J = 8.5 Hz, 1 H), 4.52-4.15 (m, |
| | 4 H), 3.80-3.75 (m, 3 H), 2.79-2.74 (m, 3 H), 2.47- |
| | 2.28 (m, 2 H), 2.11-1.88 (m, 2 H); MS (ESI+): |
| | m/z 447.4 [M + H]. |

### Example-1-19: (2S)-2-({[(9H-fluoren-9-yl)methoxy]carbonyl}amino)-3-(1H-1,2,3-triazol-4-yl)propanoic acid

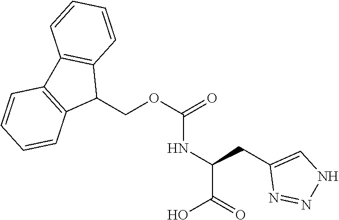 [SMILES: O=C(N[C@@H](Cc1c[nH]nn1)C(=O)O)OCC1c2ccccc2-c2ccccc21]

To a solution of 1-(azidomethyl)-2,4-dimethoxybenzene (8.11 g, 42.0 mmol) in DMF (80 mL) at rt were added (S)-2-((((9H-fluoren-9-yl)methoxy)carbonyl)amino)pent-4-ynoic acid (CAS: 198561-07-8, 13.41 g, 40 mmol), copper(II) sulfate pentahydrate (0.50 g, 2.00 mmol), and sodium (R)-2-((S)-1,2-dihydroxyethyl)-4-hydroxy-5-oxo-2,5-dihydrofuran-3-olate (15.9 g, 80 mmol). The mixture was stirred at rt for 1 h and then diluted with H₂O/satd. aq. NH₄Cl (ca. 50/50, 800 mL). The resulting solid was collected and washed with H₂O and iPr₂O. A mixture of the obtained material (20.0 g, 37.8 mmol) and TFA (87 ml, 1135 mmol) was stirred at 60° C. for 2 h, and then concentrated. The resulting residue was dissolved in CH₂Cl₂ and H₂O, and the pH of the aqueous layer was adjusted to pH 4 with NaHCO₃. The resulting solid was collected by filtration, which was then triturated with CH₂Cl₂/hexane to afford the title compound (11.2 g, 27.6 mmol). ¹H NMR (500 MHz, DMSO-d₆) δ 14.64 (br s, 1), 12.83 (br s, 1H), 7.89 (d, J=7.5 Hz, 2H), 7.74 (br s, 1H), 7.66 (dd, J=7.0, 7.0 Hz, 2H), 7.58 (br s, 1H), 7.41 (dd, J=7.0, 7.5 Hz, 2H), 7.34-7.29 (m, 2H), 4.27-4.14 (m, 4H), 3.15 (d, J=14.5 Hz, 1H), 3.01 (dd, J=14.5, 10.0 Hz, 1H). MS (ESI+): m/z 379.2 [M+H].

### Example-1-20: (2S)-2-({[(9H-fluoren-9-yl)methoxy]carbonyl}amino)-3-{4-[(prop-2-en-1-yloxy)carbonyl]phenyl}propanoic acid

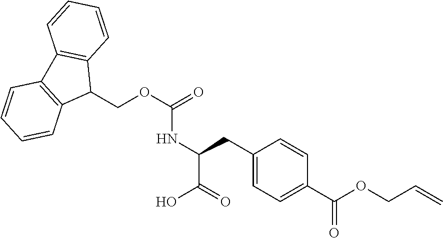 [SMILES: C=CCOC(=O)c1ccc(C[C@H](NC(=O)OCC2c3ccccc3-c3ccccc32)C(=O)O)cc1]

To a suspension of (S)-2-((((9H-fluoren-9-yl)methoxy)carbonyl)amino)-3-(4-(tert-butoxycarbonyl)phenyl)propanoic acid (CAS: 183070-44-2, 8.00 g, 16.4 mmol) in MeOH (26.0 mL) at 0° C. was added dropwise thionyl chloride (CAS: 7719-09-7, 2.52 mL, 32.8 mmol). The mixture was stirred at rt for 1 h, and then concentrated. The resulting residue was diluted with EtOAc and washed with H₂O. The mixture was extracted twice with EtOAc. The combined organic extracts were washed with satd. aq. NaHCO₃ and brine, dried over MgSO₄, filtered and concentrated. The resulting residue was purified twice by silica gel flash column chromatography (gradient, hexane/EtOAc=100/0 to 70/30). Appropriate fractions were concentrated. A mixture of the resulting residue and (3.53 g, 7.03 mmol) and TFA (43.3 mL, 563 mmol) in CH₂Cl₂ (70.3 mL) was stirred at rt for 2 h. The resulting residue was suspended in toluene, and then concentrated. To a suspension of the resulting residue (2.91 g, 6.53 mmol) in MeCN (65.3 ml) at 0° C. were added DIPEA (1.71 mL, 9.80 mmol) and allyl bromide (5.71 mL, 65.3 mmol). The mixture was stirred at rt for 20 h and then concentrated. The residue was diluted with EtOAc, washed successively with satd. aq. NaHCO₃ and brine, dried over MgSO₄, filtered, and then concentrated. The resulting residue was purified by silica gel flash column chromatography (gradient, hexane/EtOAc=100/0 to 67/33).

Appropriate fractions were concentrated. A stirred solution of the resulting residue and trimethylstannanol (3.09 g, 17.1 mmol) (2.77 g, 5.70 mmol) in ClCH₂CH₂Cl (19.0 mL) was stirred at 50° C. for 16 h. The mixture was concentrated. The residue was purified by silica gel flash column chromatography precharged with KF powder (gradient, hexane/EtOAc=50/50, then CH₂Cl₂/MeOH=100/0 to 70/30) to afford the title compound (1.94 g, 4.11 mmol). ¹H NMR (500 MHz, DMSO-d₆) δ 12.87 (br s, 1H), 7.88 (d, J=7.5 Hz, 4H), 7.61 (dd, J=7.0, 4.0 Hz, 2H), 7.44-7.36 (m, 4H), 7.33-7.24 (m, 2H), 6.08-5.98 (m, 1H), 5.38 (d, J=17.0 Hz, 1H), 5.27 (d, J=10.0 Hz, 1H), 4.78 (d, J=5.5 Hz, 2H), 4.23-4.13 (m, 4H), 3.17 (dd, J=13.5, 4.0 Hz, 1H), 2.94 (dd, J=13.0, 11.0 Hz, 1H). MS (ESI+): m/z 472.4 [M+H].

### Example-1-21: (2S)-3-{6-[(tert-butoxy)carbonyl]pyridin-3-yl}-2-({[(9H-fluoren-9-yl)methoxy]carbonyl}amino)propanoic acid

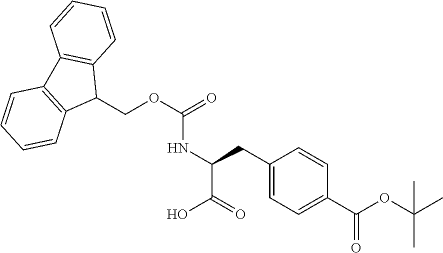 [SMILES: CC(C)(C)OC(=O)c1ccc(C[C@H](NC(=O)OCC2c3ccccc3-c3ccccc32)C(=O)O)cc1]

To a suspension of Zn (3.27 g, 50.00 mmol) in DMF (30 mL) at rt were added 1,2-dibromoethane (0.086 mL, 1.00 mmol) and chlorotrimethylsilane (0.25 mL, 2.00 mmol). The mixture was stirred at 60° C. for 30 min. To the mixture at rt was added methyl (R)-2-((((9H-fluoren-9-yl)methoxy)carbonyl)amino)-3-iodopropanoate (5.42 g, 12.00 mmol). The mixture was stirred at rt for 2 h. To a mixture of tert-butyl 5-bromopicolinate (2.58 g, 10.00 mmol), Pd2(dba)3 CHCl3 adduct (0.52 g, 0.50 mmol), and SPhos (0.41 g, 1.00 mmol) in DMF (20 mL) at 50° C. was added the organozinc reagent. The mixture was stirred at 50° C. for 2 h. The reaction was quenched with EtOAc and 2M aq. HCl at 0° C. The mixture was filtered. The filtrate was extracted with EtOAc. The organic extract was washed with brine, dried over Na2SO4, filtered, and then concentrated. The resulting residue was purified by silica gel flash column chromatography (gradient, CH2Cl2/MeOH=100/0 to 80/20), and then silica gel flash column chromatography (gradient, hexane/EtOAc=100/0 to 0/100). Appropriate fractions were concentrated. To the resulting residue (3.84 g, 7.64 mmol) in ClCH2CH2Cl (31 mL) at rt was added trimethyltin hydroxide (3.32 g, 18.3 mmol). The mixture was stirred at 40° C. for 16 h, and then concentrated. The resulting residue was purified by silica gel flash column chromatography precharged with KF powder (gradient, CH2Cl2/MeOH=100/0 to 80/20), and then silica gel flash column chromatography (gradient, hexane/EtOAc=100/0 to 50/50) to afford the title compound (1.54 g, 3.15 mmol). 1H NMR (500 MHz, DMSO-d6) δ=12.89 (br s, 1H), 8.60 (s, 1H), 7.91-7.75 (m, 5H), 7.59 (dd, J=12.5, 7.5 Hz, 2H), 7.44-7.35 (m, 2H), 7.34-7.24 (m, 2H), 4.30-4.09 (m, 4H), 3.21 (dd, J=13.8, 4.5 Hz, 1H), 2.96 (dd, J=13.8, 11.0 Hz, 1H), 1.54 (s, 9H). MS (ESI+): m/z 489.3 (M+H).

### Reference example-2-1-01: Prop-2-en-1-yl (2S)-6-amino-2-({[(9H-fluoren-9-yl)methoxy]carbonyl}(methyl)amino)hexanoate hydrochloride

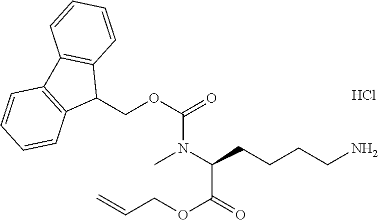 [SMILES: C=CCOC(=O)[C@H](CCCCN)N(C)C(=O)OCC1c2ccccc2-c2ccccc21 ; Cl]

To a solution of (2S)-2-[9H-fluoren-9-ylmethoxycarbonyl(methyl)amino]-6-[(2-methylpropan-2-yl)oxycarbonylamino]hexanoic acid (CAS: 197632-76-1, 12 g, 24.9 mmol) in DMF (99 mL) at rt were added DIPEA (6.51 mL, 37.3 mmol) and allyl bromide (2.37 mL, 27.4 mmol). The mixture was stirred at rt for ca. 16 h. The reaction was quenched with H₂O. The mixture was extracted with EtOAc. The organic layer was dried over Na₂SO₄, filtered and concentrated. The resulting residue was purified by silica gel flash column chromatography (gradient, hexane/EtOAc=100/0 to 50/50). Appropriate fractions were concentrated. A mixture of the resulting residue (12.1 g, 22.92 mmol) and and 2M HCl in CH₂Cl₂/CPME (ca. 1/1, 232 mL) was stirred at rt for 2 h. The mixture was concentrated to furnish the title compound (10.35 g, 22.32 mmol) which was used in the next step without any further purification. MS (ESI+): m/z 423.4 [M+H].

### Reference Example-2-1

The following compounds were synthesized as outlined for the preparation of Reference Example-2-1-01 employing appropriate starting materials in the table.

| | Structure and IUPAC name |
|---|---|
| | Starting material acid |
| Example | MS data |
| 2-1-02 | 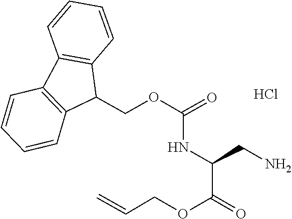 [SMILES: C=CCOC(=O)[C@H](CN)NC(=O)OCC1c2ccccc2-c2ccccc21 ; Cl] |
| | prop-2-en-1-yl (2S)-3-amino-2-({[(9H-fluoren-9-yl)methoxy] |
| | carbonyl}amino)propanoate hydrochloride |
| | (2S)-3-{[(tert-butoxy)carbonyl]amino}-2-({[(9H-fluoren-9-yl) |
| | methoxy]carbonyl}amino)propanoic acid (CAS: 162558-25-0) |
| | MS (ESI+): m/z 367.3 [M + H] |
| 2-1-03 | 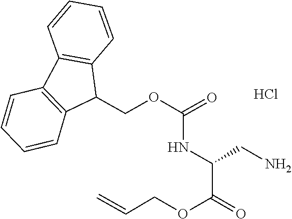 [SMILES: C=CCOC(=O)[C@@H](CN)NC(=O)OCC1c2ccccc2-c2ccccc21 ; Cl] |
| | prop-2-en-1-yl (2R)-3-amino-2-({[(9H-fluoren-9-yl)methoxy] |
| | carbonyl}amino)propanoate hydrochloride |
| | (2R)-3-{[(tert-butoxy)carbonyl]amino}-2-({[(9H-fluoren-9-yl) |
| | methoxy]carbonyl}amino)propanoic acid (CAS: 198544-42-2) |
| | Substantially identical to Reference example 2-1-02 |
| 2-1-04 | 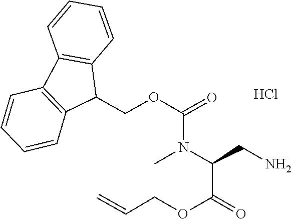 [SMILES: C=CCOC(=O)[C@H](CN)N(C)C(=O)OCC1c2ccccc2-c2ccccc21 ; Cl] |
| | prop-2-en-1-yl (2S)-3-amino-2-({[(9H-fluoren-9-yl)methoxy] |
| | carbonyl}(methyl)amino)propanoate hydrochloride |
| | (2S)-3-{[(tert-butoxy)carbonyl]amino}-2-({[(9H-fluoren-9-yl) |
| | methoxy]carbonyl}(methyl)amino)propanoic acid |
| | MS (ESI+): m/z 381.4 [M + H] |

Following compounds were prepared and used for preparations of the disclosure.

### Intermediate-2-1-01. 2,5-dioxopyrrolidin-1-yl 1-[2-(tert-butoxy)-2-oxoethyl]piperidine-4-carboxylate

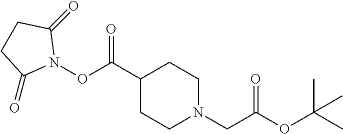 [SMILES: CC(C)(C)OC(=O)CN1CCC(C(=O)ON2C(=O)CCC2=O)CC1]

To a suspension of 1-[2-(tert-butoxy)-2-oxoethyl]piperidine-4-carboxylic acid (CAS: 193903-41-2, 5.00 g, 20.55 mmol) in CH₂Cl₂ (41 mL) at 0° C. was added 1-(3-Dimethylaminopropyl)-3-ethylcarbodiimide hydrochloride (CAS: 25952-53-8, 4.73 g, 24.66 mmol) and 1-hydroxypyrrolidine-2,5-dione (CAS: 6066-82-6, 2.84 g, 24.66 mmol). The mixture was stirred at rt for 3 h, and then the resulting mixture was purified by silica gel flash column chromatography (gradient, heptane/EtOAc=95/5 to 30/70) to afford the title compound (5.48 g, 16.11 mmol). MS (ESI+): m/z 341.3 (M+H).

### Intermediate-2-1-02: prop-2-en-1-yl 2-(piperazin-1-yl)acetate dihydrochloride

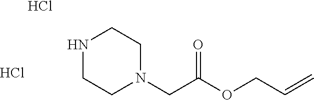

To a solution of tert-butyl piperazine-1-carboxylate (10 g, 53.7 mmol) in DMF (134 ml) at rt were added allyl 2-chloroacetate (6.10 ml, 52.6 mmol), and DIPEA (18.75 ml, 107 mmol) at rt. The mixture was stirred at 40° C. for 5 h. pH of aqueous phase was adjusted to 6 with 1M HCl aq. at 0° C. The solution was extracted twice with EtOAc. The combined organic extracts were washed with water and brine, dried over Na2SO4, filtered and concentrated. To a solution of resulting residue in THF (30 mL) at rt was added 4M hydrogen chloride in CPME (30 mL). The mixture was stirred at 30° C. for 4 h and then concentrated. The resulting solid was triturated with hexane (50 mL). The supernatant was decantated and residue was concentrated in vacuo to afford the title compound (10.5 g, 40.8 mmol). MS (ESI+): m/z 285.7 [M+H].

### Intermediate-2-1-03: 3-methylpentan-3-yl 2-(piperazin-1-yl)acetate

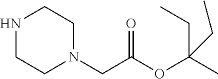 [SMILES: CCC(C)(CC)OC(=O)CN1CCNCC1]

To a solution of piperazine (4.63 g, 53.8 mmol) in THF (22.41 ml) under reflux was added 3-methylpentan-3-yl 2-bromoacetate (2 g, 8.96 mmol). The mixture was stirred under reflux for 2 h. The mixture was cooled to rt, filtered, and then concentrated. The residue was dissolved in EtOAc, washed with H₂O and brine, dried over Na₂SO₄, filtered and concentrated. The resulting residue was purified by silica gel flash column chromatography (gradient, CH2Cl2/MeOH=100/0 to 50/50) to afford the title compound (1.2 g, 5.26 mmol). MS (ESI+): m/z 229.2 [M+H].

### Intermediate-2-1-04: tert-butyl 2-(3-aminoazetidin-1-yl)acetate

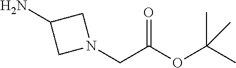

To a solution of tert-butyl 3-aminoazetidine-1-carboxylate (CAS: 193269-78-2: 1 g, 5.81 mmol) in CH₂Cl₂ (29.0 ml) was added DIPEA (1.50 g, 11.61 mmol), (9H-fluoren-9-yl)methyl (2,5-dioxopyrrolidin-1-yl) carbonate (2.15 g, 6.39 mmol) at rt. The mixture was stirred at rt for 1 h, and then quenched with water. The aqueous layer was extracted with EtOAc. The organic layer was washed with brine, dried over Na₂SO₄, filtered, and concentrated. The residue was purified by silica gel flash column chromatography (gradient, hexane/EtOAc=80/20 to 50/50) to afford tert-butyl 3-((((9H-fluoren-9-yl)methoxy)carbonyl)amino)azetidine-1-carboxylate (2.28 g, 5.78 mmol). To a solution of obtained compound (3 g, 7.61 mmol) in CH₂Cl₂ (30 mL) was added 2,2,2-trifluoroacetic acid (5.82 mL, 76 mmol) at rt. The mixture was stirred at rt for 1 h, and then concentrated. The mixture was azeotroped with toluene. The resulting residue was diluted in CH₂Cl₂ (30.0 mL). To the mixture at 0° C. were added N-ethyl-N-isopropylpropan-2-amine (5.96 mL, 34.2 mmol) and tert-butyl 2-bromoacetate (1.17 mL, 7.99 mmol). The mixture was stirred at rt for 1.5 h, and then quenched with water at 0° C. The aqueous layer was extracted twice with EtOAc. The combined organic layer was washed three times with brine, dried over Na₂SO₄, filtered and concentrated. The residue was purified by silica gel flash column chromatography (gradient, hexane/EtOAc=50/50 to 0/100) to afford tert-butyl 2-(3-((((9H-fluoren-9-yl)methoxy)carbonyl)amino)azetidin-1-yl)acetate (2.35 g, 5.75 mmol). To the mixture in THF (72 mL) at rt was added dimethylamine (28.7 mL, 57.4 mmol). The mixture was stirred at rt for 3 h, and then concentrated. The mixture was azeotroped with toluene. The residue was purified by silica gel flash column chromatography (gradient, EtOAc/MeOH=100/0 to 90/10) to afford the title compound (1.1 g, 5.88 mmol). ¹H NMR (400 MHz, CDCl3-d) δ 3.80-3.73 (m, 2H), 3.71-3.63 (m, 1H), 3.15 (s, 2H), 2.85-2.78 (m, 2H), 1.74-1.55 (2H, br), 1.46 (s, 9H)

### Intermediate-2-1-05: 2-phenylpropan-2-yl 2-(piperazin-1-yl)acetate

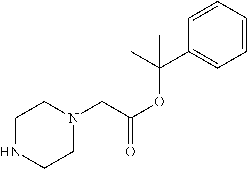 [SMILES: CC(C)(OC(=O)CN1CCNCC1)c1ccccc1]

To a refluxed solution of piperazine (7.42 g, 86 mmol) in THF (48 mL) was added dropwise a solution of 2-phenylpropan-2-yl 2-bromoacetate (CAS: 153698-47-6, 3.69 g, 14.35 mmol) in THF (24 mL) over 5 min. The mixture was stirred for 2 h under the reflux, and then cooled to rt, and then filtered. The filtrate was concentrated. The resulting residue was purified by silica gel flash column chromatography (gradient, CH₂Cl₂/MeOH=100/0 to 80/20, and then CH₂Cl₂/MeOH with 1% Et₃N=100/0 to 80/20) to afford the title compound (2.57 g, 9.81 mmol). ¹H NMR (500 MHz, DMSO-d₆) δ 7.37-7.30 (m, 4H), 7.28-7.20 (m, 1H), 3.13 (br s, 2H), 2.67-2.61 (m, 4H), 2.39-2.33 (m, 4H), 2.04 (br s, 1H), 1.69 (s, 6H).

### Intermediate-2-2-01: (5R)-5-({[(9H-fluoren-9-yl)methoxy]carbonyl}amino)-6-oxo-6-(prop-2-en-1-yloxy)hexanoic acid

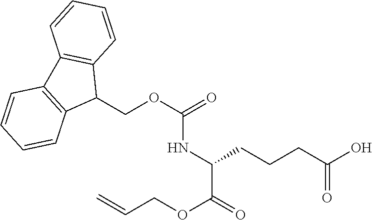 [SMILES: C=CCOC(=O)[C@@H](CCCC(=O)O)NC(=O)OCC1c2ccccc2-c2ccccc21]

To a solution of (2S)-3,6-dimethoxy-2-(propan-2-yl)-2,5-dihydropyrazine (CAS: 78342-42-4, 5.00 g, 27.14 mmol) in THF (60 mL) at −78° C. was added n-BuLi (14.00 mL, 2.5 M in hexane) dropwise. The mixture was stirred at −78° C. for 1 h. To the re action mixture at −78° C. was added a solution of tert-butyl 4-bromobutanoate (6.65 g, 29.81 mmol) in THF (20 mL) dropwise. The resulting mixture was stirred at −78° C. for an additional 3 h. The reaction was then quenched with satd. aq. NH₄Cl (20 mL).

The mixture was extracted with EtOAc. The organic extracts were washed with brine, dried over Na₂SO₄, filtered, and concentrated. The resulting residue was purified by silica gel flash column chromatography (petroleum ether/EtOAc=95/5) to afford tert-butyl 4-[(2R,5S)-3,6-dimethoxy-5-(propan-2-yl)-2,5-dihydropyrazin-2-yl]butanoate (4.50 g, 13.79 mmol). To a solution of tert-butyl 4-[(2R,5S)-3,6-dimethoxy-5-(propan-2-yl)-2,5-dihydropyrazin-2-yl]butanoate (4.50 g, 13.79 mmol), CH₃CN (54 mL) at 0° C. was added a solution of hydrogen chloride (4.5 mL) in H₂O (22.5 mL) dropwise. The mixture was stirred for 2 h at rt. The pH of the solution was adjusted to 8 with satd. aq. NaHCO₃. The mixture was extracted twice with EtOAc. The combined organic extracts were washed with brine, dried over Na₂SO₄, filtered, and concentrated. To a solution of the resulting residue in 1,4-dioxane (45 mL) and H₂O (15 mL) at 0° C. was added NaHCO₃ (2.06 g, 24.5 mmol), followed by a solution of 2, 5-dioxopyrrolidin-1-yl 9H-fluoren-9-ylmethyl carbonate (CAS: 82911-69-1, 4.56 g, 13.52 mmol) in 1,4-dioxane (15 mL) dropwise. The mixture was stirred for 2 h at rt, and then extracted with EtOAc. The organic extracts were washed with brine, dried over Na₂SO₄, filtered, and concentrated. The resulting residue was purified by silica gel flash column chromatography (petroleum ether/EtOAc=89/11). Appropriate fractions were collected and concentrated. To a solution of the residue in ClCH₂CH₂Cl (60 mL) was added trimethyltin hydroxide (4.98 g, 27.54 mmol). The mixture was stirred for 3 h at 60° C. The reaction was then quenched with satd. aq. citric acid. The mixture was extracted with EtOAc. The organic extracts were washed with brine, dried over Na₂SO₄, filtered, and concentrated. The resulting residue was purified by RP-HPLC (gradient, CH₃CN/H₂O=50/50 to 80/20) to afford (2R)-6-(tert-butoxy)-2-({[(9H-fluoren-9-yl)methoxy]carbonyl}amino)-6-oxohexanoic acid (2.21 g, 5.03 mmol). To a solution of (2R)-6-(tert-butoxy)-2-({[(9H-fluoren-9-yl)methoxy]carbonyl}amino)-6-oxohexanoic acid (1.5 g, 3.41 mmol) in DMF (8.5 mL) at rt were added DIPEA (0.89 mL, 5.12 mmol) and allyl bromide (0.38 mL, 4.4 mmol). The mixture was stirred at rt for ca. 16 h, and then diluted with H₂O. The mixture was extracted with EtOAc. The organic extracts were washed subsequently with 1M aq. HCl and brine, dried over Na₂SO₄, filtered and concentrated. The resulting residue was purified by silica gel flash column chromatography (gradient, hexane/EtOAc=100/0 to 50/50). Appropriate fractions were concentrated. A mixture of the resulting residue (1.37 g, 2.86 mmol) and TFA (0.43 mL) in CH₂Cl₂ (7 mL) was stirred for ca. 16 h at rt. The mixture was concentrated to furnish the title compound (1.19 g, 2.81 mmol) which was used in the next step without any further purification. MS (ESI+): m/z 424.3 [M+H].

### Intermediate-2-2

The following compounds were synthesized as outlined for the preparation of Intermediate-2-2-01 employing appropriate starting materials in the table.

| | Structure and IUPAC name |
|---|---|
| | Starting material acid |
| Example | MS data |
| 2-2-02 | 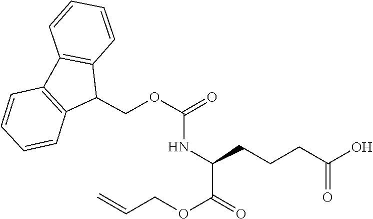 [SMILES: C=CCOC(=O)[C@H](CCCC(=O)O)NC(=O)OCC1c2ccccc2-c2ccccc21] |
| | prop-2-en-1-yl (2S)-6-amino-2-({[(9H-fluoren-9-yl)methoxy] |
| | carbonyl}(methyl)amino)hexanoate hydrochloride |
| | (2R)-3,6-dimethoxy-2-(propan-2-yl)-2,5-dihydropyrazine |
| | (CAS: 109838-85-9) |
| | Substantially identical to Intermediate-2-2-01 |

### Intermediate-2-3-01: (2S)-2-({[(9H-fluoren-9-yl)methoxy]carbonyl}amino)-6-{[(4-nitrophenoxy)carbonyl]amino}hexanoic acid

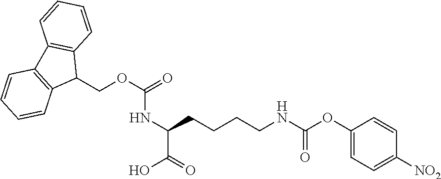 [SMILES: O=C(N[C@@H](CCCCNC(=O)Oc1ccc([N+](=O)[O-])cc1)C(=O)O)OCC1c2ccccc2-c2ccccc21]

To a suspension of (2S)-6-amino-2-({[(9H-fluoren-9-yl)methoxy]carbonyl}amino)hexanoic acid hydrochloride (1 g, 2.47 mmol) in THF (12.4 mL) at rt was added MSA (CAS: 7449-74-3, 0.79 g, 5.43 mmol). The mixture was stirred at rt for 0.5 h. To the solution at 0° C. were added DIPEA (0.52 mL, 2.96 mmol) and 4-nitrophenyl chloroformate (CAS: 7693-46-1, 0.597 g, 2.96 mmol). The mixture was stirred for 2 h at 0° C. The reaction was quenched with 2M aq. HCl. The mixture was extracted twice with EtOAc. The combined organic extracts were washed successively with H₂O twice, brine, dried over Na₂SO₄, filtered, and concentrated. The resulting residue was purified by silica gel flash chromatography (gradient, CH₂Cl₂/MeOH=98/2 to 60/40) to afford the title compound (0.77 g, 1.41 mmol). MS (ESI+): m/z 534.3 [M+H].

### Intermediate-2-3-02: (2S)-2-({[(9H-fluoren-9-yl)methoxy]carbonyl}amino)-7-{[(4-nitrophenoxy)carbonyl]amino}heptanoic acid

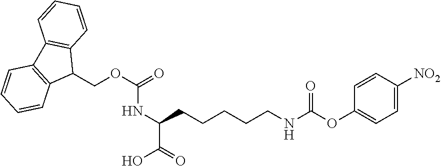 [SMILES: O=C(N[C@@H](CCCCCNC(=O)Oc1ccc([N+](=O)[O-])cc1)C(=O)O)OCC1c2ccccc2-c2ccccc21]

To a solution of (2S)-7-{[(tert-butoxy)carbonyl]amino}-2-({[(9H-fluoren-9-yl)methoxy]carbonyl}amino)heptanoic acid (10 g, 20.72 mmol) in CH₂Cl₂ (60 mL) were added 4 M HCl in CPME (60 mL). The mixture was stirred at rt for 4 h. The resulting solid was collected by filtration and washed by iPr₂O to afford (S)-2-((((9H-fluoren-9-yl)methoxy)carbonyl)amino)-7-aminoheptanoic acid hydrochloride (8.5 g, 20.29 mmol). To a suspension of the obtained compound (5 g, 11.94 mmol) in THF (60 mL) at rt was added MSA (3.81 g, 26.2 mmol). The mixture was stirred for 0.5 h at the same temperature, which turned into a clear solution. To the reaction mixture at 0° C. were added DIPEA (2.502 mL, 14.32 mmol) and 4-nitrophenyl chloroformate (2.89 g, 14.32 mmol). The mixture was stirred for 2 h at the same temperature and then quenched with 1 M aq. HCl. The mixture was extracted twice with EtOAc. The combined organic extracts were washed with H₂O, brine, dried over Na₂SO₄, filtered and concentrated. The resulting residue was suspended with MTBE. The resulted solid was collected by filtration to afford the title compound (5.5 g, 10.04 mmol). MS (ESI+): m/z 548.4 [M+H].

### Intermediate-2-3-03: (2S)-3-[4-(2-aminoethoxy)phenyl]-2-({[(9H-fluoren-9-yl)methoxy]carbonyl}amino)propanoic acid hydrochloride

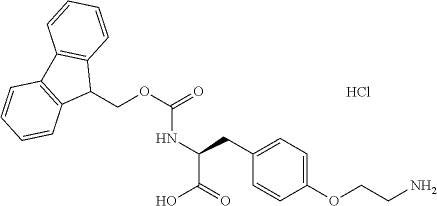 [SMILES: NCCOc1ccc(C[C@H](NC(=O)OCC2c3ccccc3-c3ccccc32)C(=O)O)cc1 ; Cl]

To a suspension of (2S)-3-[4-(2-{[(tert-butoxy)carbonyl]amino}ethoxy)phenyl]-2-({[(9H-fluoren-9-yl)methoxy]carbonyl}amino)propanoic acid (CAS: 1013883-02-7, 5.47 g, 10.00 mmol) in CH₂Cl₂ (33 mL) were added 4M HCl in MTHP (25 mL). The mixture was stirred at rt for 5 h. The resulting solid was collected by filtration and washed by iPr₂O to afford the title compound (5.086 g, 10.00 mmol). MS (ESI+): m/z 447.4 [M+H].

### Intermediate-2-3-04: (2S)-2-({[(9H-fluoren-9-yl)methoxy]carbonyl}amino)-3-[4-(2-{[(4-nitrophenoxy)carbonyl]amino}ethoxy)phenyl]propanoic acid

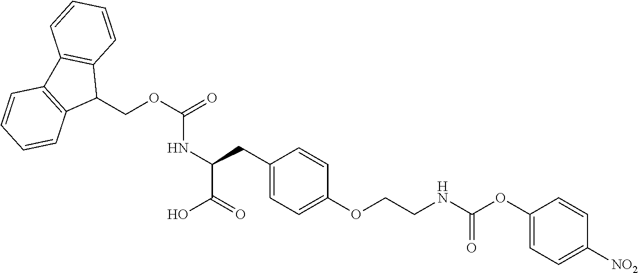 [SMILES: O=C(N[C@@H](Cc1ccc(OCCNC(=O)Oc2ccc([N+](=O)[O-])cc2)cc1)C(=O)O)OCC1c2ccccc2-c2ccccc21]

To a suspension of Intermediate-2-3-03 (4.59 g, 9.50 mmol) in THF (25 mL) at rt was added MSA (4.83 g, 23.76 mmol). The mixture was stirred for 0.5 h at the same temperature, which turned into a clear solution. To the mixture at 0° C. were added DIPEA (1.992 mL, 11.40 mmol) and 4-nitrophenyl chloroformate (1.877 g, 9.31 mmol). The mixture was stirred for 1 h at the same temperature and then quenched with 2M aq. HCl. The mixture was extracted twice with EtOAc. The combined organic extracts were washed with H₂O, brine, dried over Na₂SO₄, filtered and concentrated. The resulting residue was suspended with iPr₂O/heptane (50 mL/50 mL). The resulted solid was collected by filtration to afford the title compound (5.921 g, 9.00 mmol). MS (ESI+): m/z 612.0 [M+H].

### Intermediate-2-3-05: benzyl (2S)-2-{[(benzyloxy)carbonyl]amino}-3-[4-(2-{[2-(ter t-butoxy)-2-oxoethyl][(tert-butoxy)carbonyl]amino}ethoxy)phenyl]propanoate

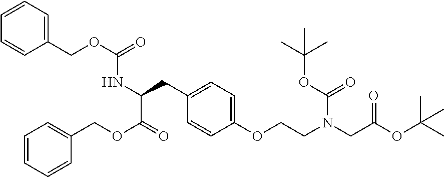 [SMILES: CC(C)(C)OC(=O)CN(CCOc1ccc(C[C@H](NC(=O)OCc2ccccc2)C(=O)OCc2ccccc2)cc1)C(=O)OC(C)(C)C]

To a solution of benzyl (2S)-2-{[(benzyloxy)carbonyl]amino}-3-(4-hydroxyphenyl)propanoate (CAS: 5513-40-6, 10.43 g, 25.72 mmol) and tert-butyl 2-{[(tert-butoxy)carbonyl](2-hydroxyethyl)amino}acetate (CAS: 1314371-47-5, 4.72 g, 17.14 mmol) in toluene (29 mL) and THF (29 mL) were added triphenylphosphine (6.74 g, 25.70 mmol) and Di-2-methoxyethyl azodicarboxylate (6.02 g, 25.70 mmol) at 0° C. The mixture was stirred at rt for 1 h and then quenched with H₂O. The mixture was extracted with toluene. The combined organic extracts were washed with H₂O, brine, dried over Na₂SO₄, filtered and concentrated. The resulting residue was purified by silica gel flash column chromatography (gradient, heptane/EtOAc=90/10 to 50/50) to afford the title compound (5.61 g, 7.11 mmol). MS (ESI+): m/z 607.5 [M−tBu+H].

### Intermediate-2-3-06: prop-2-en-1-yl (2R)-2-({[(9H-fluoren-9-yl)methoxy]carbonyl}amino)-6-{[(4-nitrophenoxy)carbonyl]amino}hexanoate

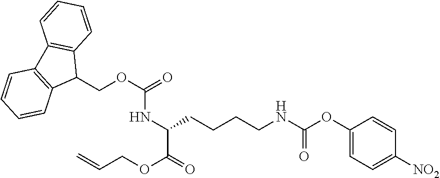 [SMILES: C=CCOC(=O)[C@@H](CCCCNC(=O)Oc1ccc([N+](=O)[O-])cc1)NC(=O)OCC1c2ccccc2-c2ccccc21]

To a solution of prop-2-en-1-yl (2R)-6-amino-2-({[(9H-fluoren-9-yl)methoxy]carbonyl}amino)hexanoate hydrochloride (5 g, 11.24 mmol) in CH₂Cl₂ (56 ml) were added 4-nitrophenyl chloroformate (2.27 g, 11.24 mmol) and DIPEA (2.55 ml, 14.61 mmol) at 0° C. The mixture was stirred at rt for 0.5 h, and then diluted with CH₂Cl₂ The mixture was quenched with sat. NaHCO₃. The organic phase was washed with brine, dried over Na₂SO₄, and concentrated. The residue was purified by silica gel flash column chromatography (gradient, hexane/EtOAc=50/50 to 0/100) to give the title compound (2.71 g, 4.72 mmol). MS (ESI+): m/z 574.4 [M+H].

### Intermediate-2-3-07: prop-2-en-1-yl (2R)-6-({[(2,5-dioxopyrrolidin-1-yl)oxy]carbonyl}oxy)-2-({[(9H-fluoren-9-yl)methoxy]carbonyl}amino)hexanoate

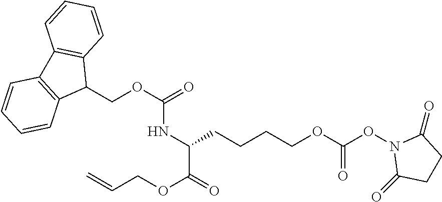 [SMILES: C=CCOC(=O)[C@@H](CCCCOC(=O)ON1C(=O)CCC1=O)NC(=O)OCC1c2ccccc2-c2ccccc21]

To a solution of prop-2-en-1-yl (2R)-6-amino-2-({[(9H-fluoren-9-yl)methoxy]carbonyl}amino)hexanoate hydrochloride (CAS: 1272754-92-3, 10 g, 22.47 mmol) in Acetonitrile/H₂O/AcOH (6/4/1, 100.1 mL) at 0° C. was added solution of sodium nitrite (23.26 g, 337 mmol) in Water (18.20 mL) dropwise with stirring in 0.5 h. The mixture was stirred at rt for 1.5 h. The reaction was then heated to 70° C. over 30 minutes. The reaction mixture was cooled to rt, and then concentrated. The residue was dissolved in H2O and extracted with EtOAc. The resulting mixture was washed with brine, dried over Na₂SO₄, filtered and concentrated. The resulting residue was purified by silica gel flash column chromatography (gradient, hexane/EtOAc=100/0 to 0/100) to afford prop-2-en-1-yl (2R)-2-({[(9H-fluoren-9-yl)methoxy]carbonyl}amino)-6-hydroxyhexanoate (4.01 g, 9.79 mmol). To a suspension of prop-2-en-1-yl (2R)-2-({[(9H-fluoren-9-yl)methoxy]carbonyl}amino)-6-hydroxyhexanoate (800 mg, 1.95 mmol) in Acetonitrile (6.51 mL) at 0° C. were added bis(2,5-dioxopyrrolidin-1-yl)carbonate (1.00 g, 3.91 mmol) and DIPEA (375 μL, 2.15 mmol). The mixture was stirred for 7 h at the same temperature and then concentrated. EtOAc was added to the residue. The resulting solid was filtered through celite pad. The filtrate was purified by silica gel flash column chromatography (gradient, hexane/EtOAc=100/0 to 80/20) to afford the title compound (886 mg, 1.61 mmol).

### Intermediate-2-3-08: (2S)-3-{1-[(tert-butoxy)carbonyl]piperidin-4-yl}-2-({[(9H-fluoren-9-yl)methoxy]carbonyl}(methyl)amino)propanoic acid

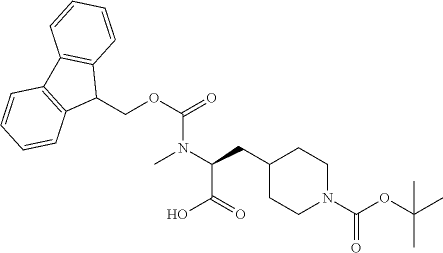 [SMILES: CN(C(=O)OCC1c2ccccc2-c2ccccc21)[C@@H](CC1CCN(C(=O)OC(C)(C)C)CC1)C(=O)O]

To a solution of (2S)-2-({[(9H-fluoren-9-yl)methoxy]carbonyl}amino)-3-{1-[(prop-2-en-1-yloxy)carbonyl]piperidin-4-yl}propanoic acid (CAS: 313052-03-8, 0.96 g, 2 mmol) and p-toluenesulfonic acid monohydrate (76 mg, 0.40 mmol) in toluene (20 mL) was added paraformaldehyde (0.275 ml, 10 mmol). The mixture was stirred at 100° C. for 3 h and then cooled to rt. The mixture was concentrated. The residue was purified by silica gel column chromatography (gradient, hexane/EtOAc=90/10 to 40/60) to afford prop-2-en-1-yl 4-{[(4S)-3-{[(9H-fluoren-9-yl)methoxy]carbonyl}-5-oxo-1,3-oxazolidin-4-yl]methyl}piperidine-1-carboxylate (1.03 g, 1.99 mmol). To a solution of the obtained compound (0.98 g, 2.0 mmol) and triisopropylsilane (3.17 g, 20 mmol) in CH₂Cl₂ (10 mL) at rt was added TFA (9.2 mL, 120 mmol). The mixture was stirred at rt for 20 h, and then concentrated. The residue was azeotroped twice with toluene. The residue was purified by silica gel column chromatography (gradient, hexane/EtOAc=90/10 to 0/100) to afford (2S)-2-({[(9H-fluoren-9-yl)methoxy]carbonyl}(methyl)amino)-3-{1-[(prop-2-en-1-yloxy)carbonyl]piperidin-4-yl}propanoic acid (889 mg, 1.766 mmol). To a solution of (2S)-2-({[(9H-fluoren-9-yl)methoxy]carbonyl}(methyl)amino)-3-{1-[(prop-2-en-1-yloxy)carbonyl]piperidin-4-yl}propanoic acid (16.5 g, 33.5 mmol) in THF (120 mL) at 0° C. were added PhSiH₃ (7.25 g, 67 mmol) and Pd(PPh₃)₄ (7.74 g, 6.7 mmol). The mixture was stirred at rt for 3 h, and then added 1,4-dioxane (60 mL), H₂O (30 mL). To the mixture were added Boc₂O (8.77 g, 40.2 mmol) and NaHCO₃ (5.63 g, 67 mmol) at 0° C. The mixture was stirred at rt for 3 h, and then quenched with critic acid aq. The mixture was extracted twice with EtOAc. The combined organic extracts were washed with brine, dried over Na₂SO₄ and concentrated. The residue was purified by silica gel column chromatography (gradient, hexane/EtOAc=99/1 to 50/50) and C18 silica gel column chromatography (gradient, H2O/MeCN with 0.1% formic acid=45/55 to 25/75) to afford the title compound (9.43 g, 18.5 mmol). MS (ESI+): m/z 409.2 [M+H].

### Intermediate-2-4-01: 4-tert-butyl 3-(9H-fluoren-9-yl)methyl (4S)-2,2-dioxo-1,2lambda6,3-oxathiazolidine-3,4-dicarboxylate

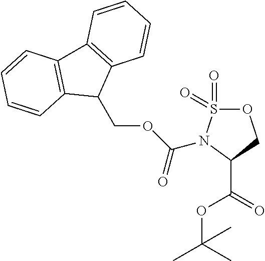 [SMILES: CC(C)(C)OC(=O)[C@@H]1COS(=O)(=O)N1C(=O)OCC1c2ccccc2-c2ccccc21]

To a solution of thionyl chloride (0.474 ml, 6.50 mmol) and MeCN (15 ml) at −40° C. was added (2S)-2-({[(9H-fluoren-9-yl)methoxy]carbonyl}amino)-3-hydroxypropanoic acid (CAS: 110797-35-8, 1.92 g, 5 mmol) in MeCN (10 ml) dropwise followed by pyridine (2.02 ml, 25 mmol) while maintaining the temperature below −30° C. The mixture was stirred at rt for 1 h, and then quenched with water. The mixture was extracted with EtOAc. The organic extract was washed with H₂O, dried over Na₂SO₄, filtered, and concentrated. To a solution of obtained residue in MeCN (30 mL) at 0° C. was added ruthenium(III) chloride (CAS: 10049-08-8, 21.0 mg, 0.100 mmol), sodium per iodate (CAS: 7790-28-5, 12.8 g, 60.0 mmol) and H₂O (100 ml). The mixture was stirred at 0° C. for 2 h, and then concentrated. The mixture was extracted with Et₂O. The organic extract was washed with NaHCO₃ aq. and brine, dried over Na₂SO₄, filtered, and concentrated. The residue was purified by flash column chromatography (gradient, hexane/EtOAc=100/0 to 0/100) to afford the title compound (1.36 g, 3.05 mmol). MS (ESI+): m/z 468 [M+Na]

### Example-2-1-01: (2S)-2-[[(9H-fluoren-9-ylmethoxy)carbonyl]amino]-6-(4-methylpiperazine-1-carbonylamino)hexanoic acid hydrochloride

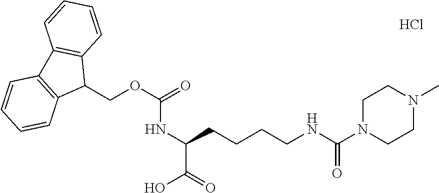 [SMILES: CN1CCN(C(=O)NCCCC[C@H](NC(=O)OCC2c3ccccc3-c3ccccc32)C(=O)O)CC1 ; Cl]

To a mixture of prop-2-en-1-yl (2S)-6-amino-2-({[(9H-fluoren-9-yl)methoxy]carbonyl}amino)hexanoate hydrochloride (CAS: 815619-80-8, 10.0 g, 22.47 mmol) in CH₂Cl₂ (50 mL) and satd. aq. NaHCO₃ (50 mL) at 0° C. was added triphosgene (CAS: 503-38-8, 2.67 g, 8.99 mmol) portionwise. The mixture was stirred for 0.5 h at the same temperature. To the mixture at 0° C. was added 1-methylpiperazine (2.25 g, 22.47 mmol) portionwise. The mixture was stirred at rt for 2 h, and then diluted with CH₂Cl₂. The mixture was washed successively with H₂O and brine, dried over Na₂SO₄, filtered, and then concentrated. The resulting residue was purified by silica gel flash column chromatography (gradient, CH₂Cl₂/MeOH=100/0 to 90/10) to afford 8.0 g of the desired product. To a solution of the product (5.00 g, 9.35 mmol) in CH₂Cl₂ (50 mL) under N₂ atmosphere were added phenylsilane (2.02 g, 18.70 mmol) and Pd(PPh₃)₄ (0.54 g, 0.47 mmol). The mixture was stirred at room temperature, and then diluted with CH₂Cl₂ (ca. 50 mL). The mixture was washed successively three times with H₂O and twice with brine, and then dried over Na₂SO₄, filtered, and then concentrated. The resulting residue was purified by silica gel flash column chromatography (gradient, CH₂Cl₂/MeOH=100/0 to 88/12). Appropriate fractions were collected and concentrated. The resulting residue was dissolved in EtOAc (20 mL), and subsequently added HCl in EtOAc (2M, 20 mL). The mixture was stirred at rt for 3 h. Resulting solid material was collected by filtration to furnish the title compound (3.18 g, 5.99 mmol). ¹H NMR (500 MHz, DMSO-d₆) δ 12.56 (br s, 1H), 10.49 (br s, 1H), 8.06 (d, J=7.63 Hz, 1H), 7.90 (d, J=7.5 Hz, 2H), 7.73 (dd, J=2.6, 7.3 Hz, 2H), 7.64 (d, J=7.9 Hz, 1H), 7.42 (dd, J=7.5, 7.5 Hz, 2H), 7.33 (dd, J=7.5, 7.5 Hz, 2H), 6.76 (br t, J=5.2 Hz, 1H), 5.20-5.11 (m, 1H), 4.32-4.21 (m, 3H), 4.15-3.94 (m, 2H), 3.94-3.84 (m, 1H), 3.18-2.97 (m, 4H), 2.96-2.85 (m, 2H), 2.74 (s, 3H), 1.74-1.54 (m, 2H), 1.46-1.26 (m, 4H). MS (ESI+): m/z 495.4 [M+H].

### Example-2-1-02: (2S)-6-({4-[2-(tert-butoxy)-2-oxoethyl]piperazine-1-carbonyl}amino)-2-({[(9H-fluoren-9-yl)methoxy]carbonyl}amino)hexanoic acid

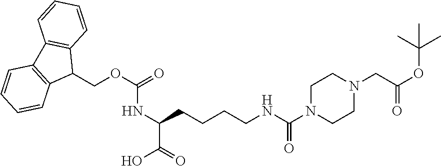 [SMILES: CC(C)(C)OC(=O)CN1CCN(C(=O)NCCCC[C@H](NC(=O)OCC2c3ccccc3-c3ccccc32)C(=O)O)CC1]

To a solution of prop-2-en-1-yl (2S)-6-amino-2-({[(9H-fluoren-9-yl)methoxy]carbonyl}amino)hexanoate hydrochloride (3.34 g, 7.5 mmol) and DIPEA (4.18 mL, 24.00 mmol) in CH₂Cl₂ (30 mL) at 0° C. was added triphosgene (1.00 g, 3.38 mmol The mixture was stirred at 0° C. for 0.25 h, and then rt for 0.75 h. To the reaction mixture at 0° C. were added tert-butyl 2-(piperazin-1-yl)acetate (CAS: 112257-22-4, 1.65 g, 8.25 mmol) and 1M aq. NaHCO₃ (50 mL). The mixture was then stirred at 0° C. for 1 h, and then extracted with EtOAc. The organic extract was washed successively with 5% aq. AcOH, H₂O, and satd. aq. NaHCO₃, dried over Na₂SO₄, filtered and concentrated.

The resulting residue was purified by silica gel flash column chromatography (gradient, hexane/EtOAc=100/0 to 0/100). Appropriate fractions were concentrated. To a solution of the resulting residue (2.13 g, 3.36 mmol) in CH₂Cl₂ (30 mL) were added phenylsilane (0.48 mL, 3.90 mmol) and Pd(PPh₃)₄ (0.04 g, 0.04 mmol) at 0° C. The mixture was then stirred at rt for 15 h, and then concentrated. The resulting residue was purified by silica gel flash column chromatography (gradient, hexane/EtOAc=50/50 to 0/100, then CH₂Cl₂/MeOH=100/0 to 85/15) to afford the title compound (1.51 g, 2.54 mmol). ¹H NMR (500 MHz, DMSO-d₆) δ 12.52 (br s, 1H), 7.90 (d, J=7.6 Hz, 2H), 7.73 (d, J=7.5 Hz, 2H), 7.68-7.55 (m, 1H), 7.42 (dd, J=7.5, 7.5 Hz, 2H), 7.33 (dd, J=7.1, 7.1 Hz, 2H), 6.45 (br t, J=5.3 Hz, 1H), 4.32-4.19 (m, 3H), 3.93-3.85 (m, 1H), 3.30-3.19 (m, 4H), 3.09 (s, 2H), 3.03-2.95 (m, 2H), 2.46-2.32 (m, 4H), 1.72-1.55 (m, 2H), 1.39 (s, 9H), 1.38-1.23 (m, 4H). MS (ESI+): m/z 595.4 (M+H)

### Example-2-1-03: (2S)-2-({[(9H-fluoren-9-yl)methoxy]carbonyl}amino)-6-({methyl[(2S,3R,4R,5R)-2,3,4,5-tetrahydroxy-6-{[tris(propan-2-yl)silyl]oxy}hexyl]carbamoyl}amino)hexanoic acid

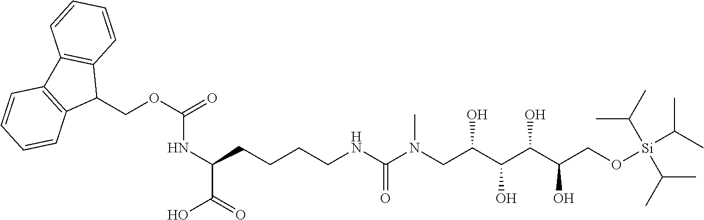 [SMILES: CC(C)[Si](OC[C@@H](O)[C@@H](O)[C@H](O)[C@@H](O)CN(C)C(=O)NCCCC[C@H](NC(=O)OCC1c2ccccc2-c2ccccc21)C(=O)O)(C(C)C)C(C)C]

To a solution of prop-2-en-1-yl (2S)-6-amino-2-({[(9H-fluoren-9-yl)methoxy]carbonyl}amino)hexanoate hydrochloride (8 g, 17.98 mmol) in CH₂Cl₂ (80 mL) were added bis(trichloromethyl) carbonate (2.67 g, 8.99 mmol) at 0° C. Then to the mixture was added DIPEA (10.05 mL, 57.5 mmol) dropwise and stirred for 0.5 h at 0° C. To the reaction mixture at 0° C. was added 1,4-dioxane (80 mL), followed by (2R,3R,4R,5S)-6-(methylamino)hexane-1,2,3,4,5-pentaol (CAS: 6284-40-8, 14.04 g, 71.9 mmol) in 0.5 M aq. NaHCO₃ (80 mL). The mixture was stirred at 0° C. for 1.5 h and then extracted with EtOAc. The organic layer was washed with H₂O, 1M aq. HCl, satd. aq. NaHCO₃, brine, dried over Na₂SO₄, filtered and concentrated. The resulting residue was purified by silica gel flash column chromatography (gradient, hexane/EtOAc=100/0 to 0/100, then CH₂Cl₂/MeOH=100/0 to 60/40) to afford prop-2-en-1-yl (2S)-2-({[(9H-fluoren-9-yl)methoxy]carbonyl}amino)-6-({methyl[(2S,3R,4R,5R)-2,3,4,5,6-pentahydroxyhexyl]carbamoyl}amino)hexanoate (8.7 g, 13.82 mmol). To a solution of prop-2-en-1-yl (2S)-2-({[(9H-fluoren-9-yl)methoxy]carbonyl}amino)-6-({methyl[(2S,3R,4R,5R)-2,3,4,5,6-pentahydroxyhexyl]carbamoyl}amino)hexanoate (3.01 g, 4.78 mmol) in CH₂Cl₂ (34.1 mL) were added imidazole (0.81 g, 11.95 mmol) and chlorotriisopropylsilane (1.27 mL, 5.98 mmol). The mixture was stirred for ca. 5 days, and then diluted with EtOAc. The organic layer was washed three times with H₂O, dried over Na₂SO₄, filtered and concentrated. The resulting residue was purified by silica gel flash column chromatography (gradient, hexane/EtOAc=100/0 to 40/60, then CH₂Cl₂/MeOH=100/0 to 90/10) to afford prop-2-en-1-yl (2S)-2-({[(9H-fluoren-9-yl)methoxy]carbonyl}amino)-6-({methyl[(2S,3R,4R,5R)-2,3,4,5-tetrahydroxy-6-{[tris(propan-2-yl)silyl]oxy}hexyl]carbamoyl}amino)hexanoate (3.16 g, 4.02 mmol).

To a solution of prop-2-en-1-yl (2S)-2-({[(9H-fluoren-9-yl)methoxy]carbonyl}amino)-6-({methyl[(2S,3R,4R,5R)-2,3,4,5-tetrahydroxy-6-{[tris(propan-2-yl)silyl]oxy}hexyl]carbamoyl}amino)hexanoate (3.16 g, 4.02 mmol) in CH₂Cl₂ (20 mL) at rt were added phenylsilane (0.99 mL, 8.04 mmol) and Pd(PPh₃)₄ (0.12 g, 0.10 mmol). The mixture was stirred for 2 h at rt, and then concentrated. The resulting residue was purified by silica gel flash column chromatography (gradient, hexane/EtOAc=100/0 to 40/60, then CH₂Cl₂/MeOH=100/0 to 70/30) to afford the title compound (2.84 g, 3.24 mmol). ¹H NMR (300 MHz, DMSO-d₆) δ 7.89 (d, J=7.5 Hz, 2H), 7.83-7.54 (m, 2H), 7.52-7.19 (m, 4H), 6.85 (s, 1H), 6.22 (s, 1H), 4.62 (d, J=5.7 Hz, 3H), 4.35-4.14 (m, 3H), 3.97-3.54 (m, 6H), 3.54-3.44 (m, 2H), 3.15-2.88 (m, 3H), 2.80 (s, 3H), 1.78-1.16 (m, 6H), 1.16-0.52 (m, 19H). MS (ESI+): m/z 746.6 [M+H].

### Example-2-1-04: (2R)-6-({[(3R)-1-[2-(tert-butoxy)-2-oxoethyl]pyrrolidin-3-yl]carbamoyl}amino)-2-({[(9H-fluoren-9-yl)methoxy]carbonyl}amino)hexanoic acid

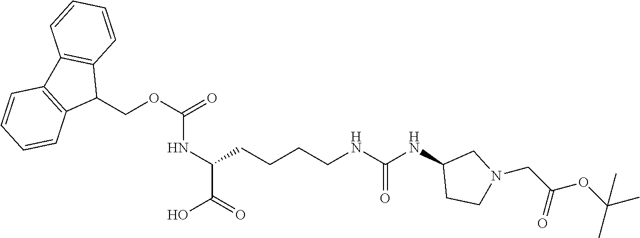 [SMILES: CC(C)(C)OC(=O)CN1CC[C@@H](NC(=O)NCCCC[C@@H](NC(=O)OCC2c3ccccc3-c3ccccc32)C(=O)O)C1]

To a suspension of prop-2-en-1-yl (2R)-6-amino-2-({[(9H-fluoren-9-yl)methoxy]carbonyl}amino)hexanoate hydrochloride (CAS: 1272754-92-3, 3 g, 6.74 mmol) in CH₂Cl₂ (27.0 mL) at 0° C. was added triphosgene (0.90 g, 3.03 mmol), followed by DIPEA (3.77 mL, 21.58 mmol) at 0° C. dropwise over 2 min. The mixture was stirred at 0° C. for 0.25 h. To the reaction mixture at 0° C. were added sodium bicarbonate (2.27 g, 27.0 mmol) and tert-butyl (R)-3-aminopyrrolidine-1-carboxylate (3.14 g, 16.86 mmol). The mixture was stirred for ca. 0.25 h at rt, and then extracted twice with CH₂Cl₂.

The combined organic extracts were washed three times with 1N HCl, H₂O, satd. aq. NaHCO₃, dried over Na₂SO₄, filtered and concentrated. The resulting residue was purified by silica gel flash column chromatography (gradient, hexane/EtOAc=6/4 to 0/1).

Appropriate fractions were concentrated. To a solution of the resulting residue (3.05 g, 4.91 mmol) in CH₂Cl₂ (19 mL) at 0° C. was added 4M HCl in CPME (3.6 mL, 14.50 mmol). The mixture was stirred for ca. 16 h at rt and then concentrated. The resulting residue was suspended in toluene and then concentrated. To the resulting residue in CH₂Cl₂ (19 mL) at 0° C. were added DIPEA (2.11 mL, 12.08 mmol) and tert-butyl bromoacetate (0.786 mL, 5.32 mmol) dropwise. The mixture was stirred for 3 h at 0° C. and then diluted with CH₂Cl₂. The organic phase was washed successively with satd. aq. NaHCO₃, brine, dried over Na₂SO₄, filtered, and concentrated. The resulting residue was purified by silica gel flash column chromatography (gradient, CH₂Cl₂/MeOH=100/0 to 90/10) to afford prop-2-en-1-yl (2R)-6-({[(3R)-1-[2-(tert-butoxy)-2-oxoethyl]pyrrolidin-3-yl]carbamoyl}amino)-2-({[(9H-fluoren-9-yl)methoxy]carbonyl}amino)hexanoate (1.70 g, 2.68 mmol).

To a solution of prop-2-en-1-yl (2R)-6-({[(3R)-1-[2-(tert-butoxy)-2-oxoethyl]pyrrolidin-3-yl]carbamoyl}amino)-2-({[(9H-fluoren-9-yl)methoxy]carbonyl}amino)hexanoate (1.70 g, 2.68 mmol) in CH₂Cl₂ (11 mL) at 0° C. were added phenylsilane (0.66 mL, 5.36 mmol) and Pd(PPh₃)₄ (0.062 g, 0.054 mmol). The mixture was stirred for 1 h at rt, and then concentrated. The residue was purified by silica gel flash column chromatography (gradient, CH₂Cl₂/MeOH=100/0 to 60/40) to afford the title compound (1.51 g, 2.52 mmol). ¹H NMR (500 MHz, DMSO-d₆) δ 12.60 (br s, 1H), 7.90 (d, J=7.5 Hz, 2H), 7.72 (d, J=7.2 Hz, 2H), 7.42 (dd, J=7.3, 7.3 Hz, 2H), 7.33 (dd, J=7.4, 7.4 Hz, 2H), 5.93 (d, J=7.5 Hz, 1H), 5.80-5.75 (m, 1H), 4.32-4.20 (m, 3H), 4.05-3.99 (m, 1H), 3.91-3.75 (m, 1H), 3.15 (s, 2H), 3.00-2.90 (m, 2H), 2.75-2.63 (m, 2H), 2.48-2.32 (m, 3H), 1.69-1.67 (m, 1H), 1.62-1.52 (m, 1H), 1.43-1.41 (m, 2H), 1.40 (s, 9H), 1.36-1.27 (m, 3H). MS (ESI+): m/z 595.6 [M+H].

### Example-2-1

The following compounds were synthesized as outlined for the preparation of Example-2-1-01, Example-2-1-02, Example-2-1-03, or Example-2-1-04 employing appropriate starting materials in the table.

| | Structure and IUPAC name |
|---|---|
| | Fmoc-protected starting material-A |
| | Starting material-B |
| Example | NMR and MS |
| 2-1-05 | 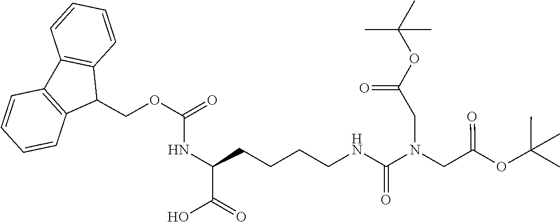 [SMILES: CC(C)(C)OC(=O)CN(CC(=O)OC(C)(C)C)C(=O)NCCCC[C@H](NC(=O)OCC1c2ccccc2-c2ccccc21)C(=O)O] |
| | (2S)-6-({bis[2-(tert-butoxy)-2-oxoethyl]carbamoyl}amino)-2-({[(9H- |
| | fluoren-9-yl)methoxy]carbonyl}amino)hexanoic acid |
| | prop-2-en-1-yl (2S)-6-amino-2-({[(9H-fluoren-9-yl)methoxy]carbonyl} |
| | amino)hexanoate hydrochloride |
| | tert-butyl 2-[[2-(tert-butoxy)-2-oxoethyl]amino]acetate (CAS: 85916-13-8) |
| | ¹H NMR (500 MHz, DMSO-d₆) δ 12.55 (br s, 1H), 7.89 (d, J = 7.63, |
| | 2H), 7.73 (d, J = 7.48 , 2H), 7.66-7.52 (m, 1H), 7.47-7.38 (m, 2H), |
| | 7.37-7.25 (m, 2H) 6.50 (t, J = 5.3 Hz, 1H), 4.34-4.15 (m, 3H), |
| | 4.00-3.80 (m, 5H), 3.04-2.92 (m, 2H), 1.77-1.54 (m, 2H), 1.47- |
| | 1.06 (m, 22H). MS (ESI+): m/z 640.3 [M + H]. |

### Example-2-2-01: (2S)-2-({[(9H-fluoren-9-yl)methoxy]carbonyl}amino)-6-[(4-{2-oxo-2-[(2-phenylpropan-2-yl)oxy]ethyl}piperazine-1-carbonyl)amino]hexanoic acid

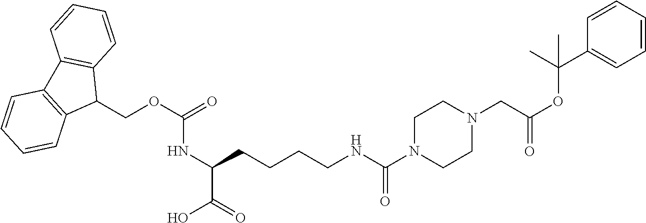 [SMILES: CC(C)(OC(=O)CN1CCN(C(=O)NCCCC[C@H](NC(=O)OCC2c3ccccc3-c3ccccc32)C(=O)O)CC1)c1ccccc1]

To a solution of Intermediate-2-3-01 (4.98 g, 9.34 mmol) in THF (28 mL) at 0° C. was added dropwise a solution of Intermediate-2-1-05 (2.57 g, 9.81 mmol) and DIPEA (1.80 mL, 10.27 mmol) in DMF (3.1 mL). The mixture was stirred at 30° C. for 3 h. The reaction at rt was quenched with 2M aq. NaH₂PO₄ (30 mL). The mixture was extracted twice with EtOAc/THF (ca. 5/1, 30 mL). The combined organic extracts were washed with brine, dried over Na₂SO₄, filtrated, and concentrated. The resulting residue was purified by silica gel flash chromatography (gradient, CH₂Cl₂/MeOH=100/0 to 92/8) to afford the title compound (3.32 g, 5.06 mmol). ¹H NMR (500 MHz DMSO-d₆) δ 12.57 (br s, 1H), 7.89 (d, J=7.5 Hz, 2H), 7.72 (d, J=7.5 Hz, 2H), 7.61 (m, 1H), 7.42 (dd, J=7.0, 7.5 Hz, 2H), 7.33 (m, 6H), 7.23 (t, J=6.5 Hz, 1H), 6.45 (s, 1H), 4.27-4.20 (m, 3H), 3.89 (m, 1H), 3.25-3.19 (m, 6H), 3.01-2.97 (m, 2H), 2.42-2.38 (m, 4H), 1.72-1.56 (m, 8H), 1.69-1.56 (m, 2H), 1.39-1.29 (m, 2H) MS (ESI+): m/z 657.6 [M+H].

### Example-2-2

The following compounds were synthesized as outlined for the preparation of Example-2-2-01 employing appropriate starting materials in the table.

| | Structure and IUPAC name |
|---|---|
| | Fmoc-protected starting material-A |
| | Starting material-B |
| Example | NMR and MS |
| 2-2-02 | 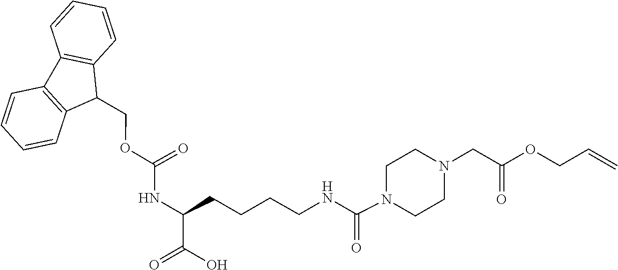 [SMILES: C=CCOC(=O)CN1CCN(C(=O)NCCCC[C@H](NC(=O)OCC2c3ccccc3-c3ccccc32)C(=O)O)CC1] |
| | (2S)-2-({[(9H-fluoren-9-yl)methoxy]carbonyl}amino)-6-({4-[2-oxo- |
| | 2-(prop-2-en-1-yloxy)ethyl]piperazine-1-carbonyl}amino)hexanoic acid |
| | Intermediate-2-3-01 |
| | Intermediate-2-1-02 |
| | ¹H NMR (500 MHz, DMSO-d₆) δ 12.55 (br s, 1H), 7.90 (d, J = |
| | 7.63 Hz, 2H) 7.73 (d, J = 7.48 Hz, 2H) 7.64 (br d, J = 7.93 Hz, 1H) |
| | 7.42 (t, J = 7.48 Hz, 2H) 7.33 (t, J = 7.48 Hz, 2H) 7.27-7.23 (m, |
| | 1H) 6.47 (br t, J = 5.26 Hz, 1H) 5.96-5.82 (m, 1H) 5.30 (br d, J = 17.24 |
| | Hz, 1H) 5.21 (d, J = 10.53 Hz, 1H) 4.56 (d, J = 5.34 Hz, 2H) |
| | 4.33-4.16 (m, 1H) 3.96-3.90 (m, 1H) 3.30-3.22 (m, 6H) 3.40-2.94 |
| | (m, 2H) 2.43 (br t, J = 4.50 Hz, 4H) 1.73-1.54 (m, 2H) 1.44- |
| | 1.25 (m, 4H), MS (ESI+): m/z 579.4 [M + H]. |
| 2-2-03 | 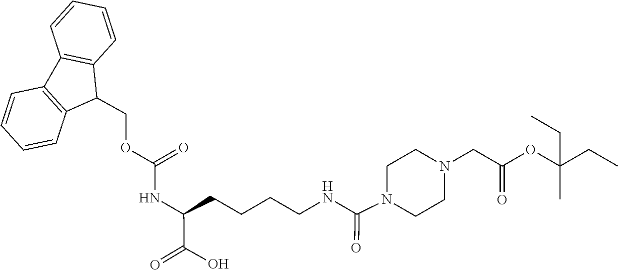 [SMILES: CCC(C)(CC)OC(=O)CN1CCN(C(=O)NCCCC[C@H](NC(=O)OCC2c3ccccc3-c3ccccc32)C(=O)O)CC1] |
| | (2S)-2-({[(9H-fluoren-9-yl)methoxy]carbonyl}amino)-6[(4-{2-[(3- |
| | methylpentan-3-yl)oxy]-2-oxoethyl}piperazine-1-carbonyl)amino]hexanoic acid |
| | Intermediate-2-3-01 |
| | Intermediate-2-1-03 |
| | ¹H NMR (500 MHz, DMSO-d₆) δ 12.56 (br s, 1H) 7.90 (d, J = |
| | 7.48 Hz, 2H) 7.73 (d, J = 7.48 Hz, 2H) 7.63 (d, J = 7.93 Hz, 1H), 7.42 |
| | (t, J = 7.40 Hz, 2H) 7.33 (t, J = 7.40 Hz, 2H) 6.46 (br t, J = 5.34 |
| | Hz, 1H) 4.30-4.20 (m, 3H) 3.93-3.87 (m, 1H) 3.28-3.21 (m, |
| | 4H) 3.12 (s, 2H) 3.06-2.93 (m, 2H) 2.42 (br t, J = 4.65 Hz, 4H) |
| | 1.85-1.76 (m, 2H) 1.73-1.53 (m, 4H) 1.43-1.26 (m, 7H) 0.79 (t, J = |
| | 7.48 Hz, 6H), MS (ESI+): m/z 746.6 [M + H]. |

### Example-2-3-01: (2S)-6-({1-[2-(tert-butoxy)-2-oxoethyl]piperidin-4-yl}formamido)-2-({[(9H-fluoren-9-yl)methoxy]carbonyl}amino)hexanoic acid

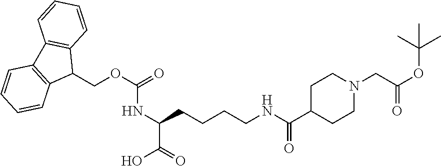 [SMILES: CC(C)(C)OC(=O)CN1CCC(C(=O)NCCCC[C@H](NC(=O)OCC2c3ccccc3-c3ccccc32)C(=O)O)CC1]

To a solution of prop-2-en-1-yl (2S)-6-amino-2-({[(9H-fluoren-9-yl)methoxy]carbonyl}amino)hexanoate hydrochloride (10.00 g, 22.47 mmol) in DMF (100 mL) at rt were added HATU (10.25 g, 26.97 mmol), DIPEA (5.81 g, 44.95 mmol) and 1-(tert-butoxycarbonyl)piperidine-4-carboxylic acid (CAS: 84358-13-4, 6.18 g, 26.97 mmol). The mixture was stirred at rt for 4 h, and then diluted with H₂O. The mixture was extracted twice with EtOAc. The combined organic extracts were washed three times with brine, dried over Na₂SO₄, filtered, and concentrated. The resulting residue was purified by silica gel flash column chromatography (EtOAc/petroleum ether=50/50). Appropriate fractions were collected and concentrated. To the resulting residue in CH₂Cl₂ (80 mL) was added TFA (80 mL). The mixture was stirred at rt for 2 h, and then concentrated. To the resulting residue in DMF (100 mL) were added DIPEA (8.37 g, 64.76 mmol) and tert-butyl 2-bromoacetate (4.74 g, 24.29 mmol). The mixture was stirred at rt for ca. 16 h, and then diluted with H₂O. The mixture was extracted twice with EtOAc. The combined organic extracts were washed twice with brine, dried over Na₂SO₄, filtered, and then concentrated. The resulting residue was purified by silica gel flash column chromatography (gradient, petroleum ether/EtOAc=100/0 to 0/100). Appropriate fractions were combined and concentrated. The resulting residue was dissolved in MeOH (100 mL), To a flask charged with Pd/C (2.0 g) under N₂ atmosphere at 0° C. was added the MeOH solution, followed by triethylsilane (18.35 g, 157.78 mmol). The mixture was stirred at rt for 1 h, and then filtered, and then concentrated. The resulting residue was purified by silica gel flash column chromatography (CH₂Cl₂/MeOH=92/8) to afford the title compound (6.32 g, 10.64 mmol). ¹H NMR (300 MHz, DMSO-d₆) δ 7.88 (d, J=7.5 Hz, 2H), 7.72 (d, J=7.7 Hz, 3H), 7.51-7.28 (m, 4H), 4.24 (q, J=6.1, 4.7 Hz, 3H), 3.88 (s, 1H), 3.16 (s, 2H), 3.02 (d, J=6.5 Hz, 4H), 2.78 (d, J=11.0 Hz, 2H), 2.19-1.92 (m, 3H), 1.78-1.48 (m, 6H), 1.39 (s, 13H). MS (ESI+): m/z 594.3 [M+H].

### Example-2-3-02: (2R)-6-{1-[2-(tert-butoxy)-2-oxoethyl]piperidine-4-sulfonamido}-2-({[(9H-fluoren-9-yl)methoxy]carbonyl}amino)hexanoic acid

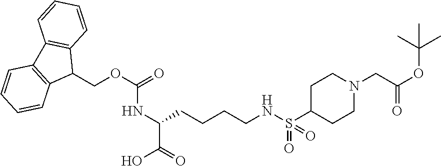 [SMILES: CC(C)(C)OC(=O)CN1CCC(S(=O)(=O)NCCCC[C@@H](NC(=O)OCC2c3ccccc3-c3ccccc32)C(=O)O)CC1]

To a solution of prop-2-en-1-yl (2R)-6-amino-2-({[(9H-fluoren-9-yl)methoxy]carbonyl}amino)hexanoate hydrochloride (2.0 g, 4.49 mmol) in CH₂Cl₂ (45 mL) at rt were added DIPEA (3.1 mL, 17.98 mmol) and tert-butyl 4-(chlorosulfonyl)piperidine-1-carboxylate (1.8 g, 6.29 mmol). The mixture was stirred for ca. 16 h at the same temperature and then diluted with H₂O. The mixture was extracted twice with CH₂Cl₂. The combined organic layer was washed with brine, dried over Na₂SO₄, filtered, and then concentrated. The resulting residue was purified by silica gel flash column chromatography (gradient, hexane/EtOAc=80/20 to 20/80). Appropriate fractions were concentrated. To a solution of the resulting residue (1.35 g, 2.06 mmol) in 1,4-dioxane (10.3 mL) was added 4M HCl in 1,4-dioxane (15.4 mL) at rt. The mixture was stirred for ca. 16 h at the same temperature and then concentrated. The resulting residue was used in the next step without any further purification. To a solution of the resulting residue in DMF (10.3 mL) at rt were added DIPEA (1.43 mL, 8.23 mmol) and tert-butyl 2-bromoacetate (0.60 g, 3.09 mmol). The mixture was stirred for ca. 16 h at the same temperature, and then diluted at 0° C. with H₂O. The mixture was extracted twice with EtOAc. The combined organic layer was washed twice with brine, dried over Na₂SO₄, filtered and concentrated. The resulting residue was purified by silica gel flash column chromatography (gradient, heptane/EtOAc=80/20 to 20/80). Appropriate fractions were concentrated. To a solution of the resulting residue (1.47 g, 2.20 mmol) in CH₂Cl₂ (29 mL) at 0° C. were added phenylsilane (0.63 g, 5.78 mmol) and Pd(PPh₃)₄ (0.08 g, 0.07 mmol). The mixture was stirred at 0° C. for 1 h, and then concentrated. The resulting residue was purified by silica gel flash column chromatography (gradient, CH₂Cl₂/MeOH=100/0 to 85/15) to afford the title compound (1.691 g, 2.69 mmol). ¹H NMR (500 MHz, DMSO-d₆) δ 12.73-12.52 (m, 1H), 7.90 (br d, J=7.5 Hz, 2H), 7.73 (br d, J=6.9 Hz, 2H), 7.66-7.59 (m, 1H), 7.42 (dd, J=7.4, 7.4 Hz, 2H), 7.33 (t, J=7.3 Hz, 2H), 7.05 (t, J=5.8 Hz, 1H), 4.31-4.21 (m, 3H), 3.94-3.87 (m, 1H), 3.09 (s, 2H), 2.96-2.83 (m, 5H), 2.19 (br t, J=11.2 Hz, 2H), 1.89 (br d, J=11.0 Hz, 2H), 1.73-1.51 (m, 4H), 1.49-1.27 (m, 4H), 1.40 (s, 9H). MS (ESI+): m/z 630.4 [M+H].

### Example-2-3-03: (2S)-3-[4-(2-{4-[2-(tert-butoxy)-2-oxoethyl]piperazin-1-yl}-2-oxoethoxy)phenyl]-2-({[(9H-fluoren-9-yl)methoxy]carbonyl}amino)propanoic acid

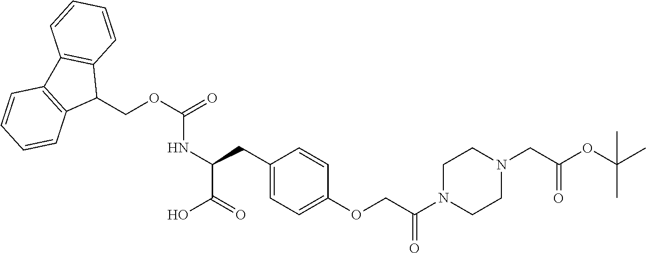 [SMILES: CC(C)(C)OC(=O)CN1CCN(C(=O)COc2ccc(C[C@H](NC(=O)OCC3c4ccccc4-c4ccccc43)C(=O)O)cc2)CC1]

To a solution of (2S)-3-{4-[2-(tert-butoxy)-2-oxoethoxy]phenyl}-2-({[(9H-fluoren-9-yl)methoxy]carbonyl}amino)propanoic acid (CAS: 181951-92-8, 40 g, 77.28 mmol) in DMF (400 mL) at 0° C. were added NaHCO₃ (13.00 g, 154.57 mmol) and 3-bromoprop-1-ene (11.20 g, 92.74 mmol). The mixture was stirred at rt for 2 h, and then diluted with H₂O. The mixture was extracted twice with EtOAc. The combined organic extracts were washed three times with brine, dried over Na₂SO₄, filtrated, and then concentrated. The resulting residue was purified by silica gel column chromatography (petroleum ether/EtOAc=50/50). Appropriate fractions were concentrated. To a solution of the resulting residue (31 g, 55.60 mmol) in CH₂Cl₂ (300 mL) at 0° C. was added TFA (150 mL). The mixture was stirred at rt for 2 h, and then concentrated. The resulting residue was suspended in toluene, and then concentrated. To a solution of the resulting residue in DMF (400 mL) at rt were added tert-butyl 2-(piperazin-1-yl)acetate (13.609 g, 67.983 mmol), HATU (47.00 g, 123.63 mmol), and DIPEA (31.93 g, 247.26 mmol). The mixture was stirred at rt for 3 h, and then diluted with H₂O. The mixture was extracted three times with EtOAc. The combined organic extracts were washed with three times with brine, dried over Na₂SO₄, filtered, and then concentrated. The resulting residue was purified by silica gel flash column chromatography (petroleum ether/EtOAc=50/50). Appropriate fractions were concentrated. To a solution of the resulting residue (23 g, 33.64 mmol) in THF at rt were added morpholine (5.86 g, 67.26 mmol) and Pd(PPh₃)₄ (3.89 g, 3.36 mmol). The mixture was stirred at rt for 0.5 h. The reaction was quenched with H₂O. The pH of the mixture was adjusted to 6 with citric acid. The mixture was extracted three times with EtOAc. The combined organic extracts were washed three times with brine, dried over Na₂SO₄, filtrated, and then concentrated. The resulting residue was purified by reverse-phase flash chromatography (Column, C18 silica gel; gradient, CH₃CN/H₂O=10/90 to 50/50) to afford the title compound (11.0 g, 17.1 mmol). ¹H NMR (400 M Hz, DMSO-d₆) δ 12.68 (br s, 1H), 7.88 (d, J=7.2 Hz, 2H), 7.73-7.37 (m, 8H), 7.36-7.24 (m, 2H), 7.17 (d, J=8.4 Hz, 2H), 6.90-6.75 (m, 2H), 4.73 (s, 2H), 4.25-4.06 (m, 4H), 3.61 (br s, 1H), 3.43 (br s, 4H), 3.14 (s, 2H), 3.01 (dd, J=13.8, 4.0 Hz, 1H), 2.80 (dd, J=13.8, 10.4 Hz, 1H), 1.40 (s, 9H). MS (ESI+): m/z 644.2 [M+H].

### Example-2-3

The following compounds were synthesized as outlined for the preparation of Example-2-3-01, Example-2-3-02, or Example-2-3-03 employing appropriate starting materials in the table.

| | Structure and IUPAC name |
|---|---|
| | Fmoc-protected starting material-A |
| | Starting material-B |
| Example | NMR and MS |
| 2-3-04 | 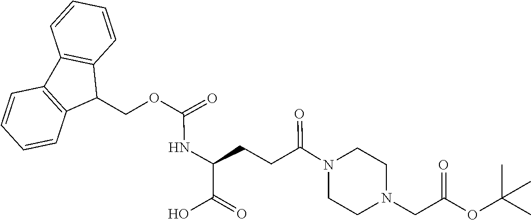 [SMILES: CC(C)(C)OC(=O)CN1CCN(C(=O)CC[C@H](NC(=O)OCC2c3ccccc3-c3ccccc32)C(=O)O)CC1] |
| | (2S)-5-{4-[2-(tert-butoxy)-2-oxoethyl]piperazin-1-yl}-2-({[(9H-fluoren-9- |
| | yl)methoxy]carbonyl}amino)-5-oxopentanoic acid |
| | (4S)-4-({[(9H-fluoren-9-yl)methoxy]carbonyl}amino)-5-oxo-5-(prop-2-en- |
| | 1-yloxy)pentanoic acid (CAS: 144120-54-7) |
| | tert-butyl 2-(piperazin-1-yl)acetate |
| | ¹H NMR (500 MHz, Methanol-d₄) δ 7.79 (d, J = 7.6 Hz, 2H), 7.70- |
| | 7.62 (m, 2H), 7.38 (dd, J = 7.5, 7.5 Hz, 2H), 7.30 (dd, J = 7.5, 7.5 Hz, |
| | 2H), 4.40-4.30 (m, 2H), 4.24-4.19 (m, 2H), 3.71-3.56 (m, 4H), 3.40 |
| | (s, 2H), 2.81-2.73 (m, 4H), 2.55-2.42 (m, 2H), 2.24-2.17 (m, |
| | 1H), 1.98-1.90 (m, 1H), 1.47 (s, 9H). MS (ESI+): m/z 552.3 [M + H]. |
| 2-3-05 | 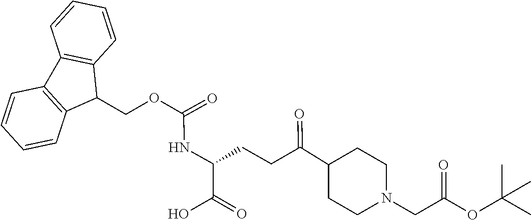 [SMILES: CC(C)(C)OC(=O)CN1CCC(C(=O)CC[C@@H](NC(=O)OCC2c3ccccc3-c3ccccc32)C(=O)O)CC1] |
| | (2R)-3-({1-[2-(tert-butoxy)-2-oxoethyl]piperidin-4-yl}formamido)-2- |
| | ({[(9H-fluoren-9-yl)methoxy]carbonyl}amino)propanoic acid |
| | Reference example 2-1-03 |
| | 1-(tert-butoxycarbonyl)piperidine-4-carboxylic acid |
| | ¹H NMR (500 MHz, CDCl₃) δ 7.80-7.67 (m, 2H), 7.67-7.53 (m, 2H), |
| | 7.41-7.32 (m, 2H), 7.32-7.24 (m, 2H), 7.16 (br s, 1H), 6.53 (br s, |
| | 1H), 4.52-4.08 (m, 4H), 3.93-3.65 (m, 2H), 3.43-3.15 (m, 4H), |
| | 2.63 (br s, 2H), 2.29 (m, 1H), 2.07-1.74 (m, 4H), 1.42 (s, 9H). MS |
| | (ESI+): m/z 552.5 [M + H]. |
| 2-3-06 | 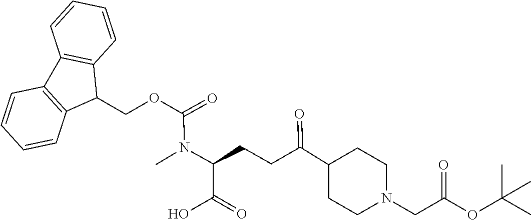 [SMILES: CN(C(=O)OCC1c2ccccc2-c2ccccc21)[C@@H](CCC(=O)C1CCN(CC(=O)OC(C)(C)C)CC1)C(=O)O] |
| | (2S)-3-({1-[2-(tert-butoxy)-2-oxoethyl]piperidin-4-yl}formamido)-2- |
| | ({[(9H-fluoren-9-yl)methoxy]carbonyl}(methyl)amino)propanoic acid |
| | Reference example 2-1-04 |
| | 1-(tert-butoxycarbonyl)piperidine-4-carboxylic acid |
| | ¹H NMR (500 MHz, DMSO-d₆) δ 13.0 (br s, 1H), 8.11-7.96 (m, 1H), |
| | 7.93-7.89 (m, 2H), 7.68-7.58 (m, 2H), 7.48-7.26 (m, 4H), 4.91- |
| | 4.60 (m, 1H), 4.36-4.07 (m, 3H), 3.74-3.92 (m, 8H), 2.83 (s, 3H), 2.32- |
| | 2.01 (m, 1H), 1.82-1.56 (m, 4H), 1.42 (s, 9H). MS (ESI+): m/z 566.3 |
| | [M + H]. |
| 2-3-07 | 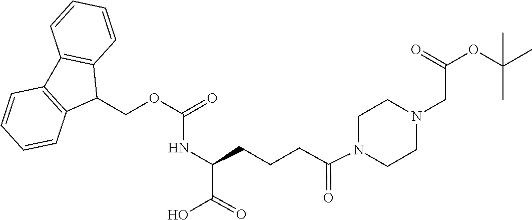 [SMILES: CC(C)(C)OC(=O)CN1CCN(C(=O)CCC[C@H](NC(=O)OCC2c3ccccc3-c3ccccc32)C(=O)O)CC1] |
| | (2S)-6-{4-[2-(tert-butoxy)-2-oxoethyl]piperazin-1-yl}-2-({[(9H-fluoren-9- |
| | yl)methoxy]carbonyl}amino)-6-oxohexanoic acid |
| | Intermediate-2-2-02: |
| | tert-butyl 2-(piperidin-1-yl)acetate |
| | ¹H NMR (500 MHz, DMSO-d₆) δ 12.60 (br s, 1H), 7.90 (d, J = 7.5 Hz, |
| | 2H), 7.73 (d, J = 7.5 Hz, 2H), 7.71-7.50 (m, 1H), 7.42 (dd, J = 7.5, |
| | 7.5 Hz, 2H), 7.33 (dd, J = 7.5, 7.5 Hz, 2H), 4.27-4.21 (m, 3H), 3.94- |
| | 3.89 (m, 1H), 3.42-3.39 (m, 4H), 3.12 (s, 2H), 2.50-2.45 (m, 2H), |
| | 2.44-2.40 (m, 2H), 2.33-2.24 (m, 2H), 1.74-1.71 (m, 1H), 1.62- |
| | 1.52 (m, 3H), 1.40 (s, 9H). MS (ESI+): m/z 566.5 [M + H]. |
| 2-3-08 | 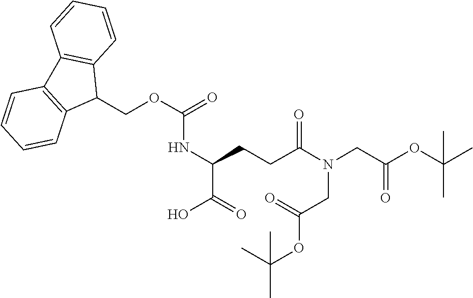 [SMILES: CC(C)(C)OC(=O)CN(CC(=O)OC(C)(C)C)C(=O)CC[C@H](NC(=O)OCC1c2ccccc2-c2ccccc21)C(=O)O] |
| | (2S)-4-{bis[2-(tert-butoxy)-2-oxoethyl]carbamoyl}-2-({[(9H-fluoren-9-yl) |
| | methoxy]carbonyl}amino)butanoic acid |
| | (4S)-4-({[(9H-fluoren-9-yl)methoxy]carbonyl}amino)-5-oxo-5-(prop-2-en- |
| | 1-yloxy)pentanoic acid (CAS: 144120-54-7) |
| | tert-butyl 2-[[2-(tert-butoxy)-2-oxoethyl]amino]acetate (CAS: 85916-13-8) |
| | ¹H NMR (400 MHz, DMSO-d6) δ 12.58 (br s, 1H), 7.90 (d, J = 7.48 Hz, |
| | 2H) 7.33 (br d, J = 7.48 Hz, 2H) 7.69-7.61 (m, 1H) 7.46-7.39 (m, |
| | 2H) 7.37-7.30 (m, 2H) 4.37-4.17 (m, 3H) 4.12 (s, 2H) 4.03-3.97 |
| | (m, 1H) 3.96-3.87 (s, 2H) 2.45-2.23 (m, 2H) 1.98-1.92 (m, 1H) |
| | 1.89-1.77 (m, 1H) 1.43-1.36 (m, 18H). MS (ESI+): m/z 597.2 [M + H]. |

### Example-2-4-01: (2S)-3-{1-[2-(tert-butoxy)-2-oxoethyl]piperidin-4-yl}-2-({[(9H-fluoren-9-yl)methoxy]carbonyl}amino)propanoic acid

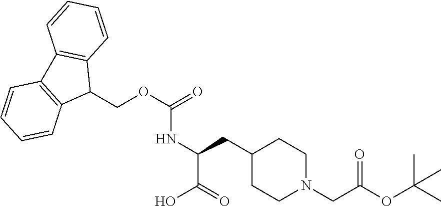 [SMILES: CC(C)(C)OC(=O)CN1CCC(C[C@H](NC(=O)OCC2c3ccccc3-c3ccccc32)C(=O)O)CC1]

To a solution of (S)-2-((((9H-fluoren-9-yl)methoxy)carbonyl)amino)-3-(1-(tert-butoxycarbonyl)piperidin-4-yl)propanoic acid (7.5 g, 15.16 mmol) in CH₂Cl₂ (60 mL) at 0° C. was added 4M hydrogen chloride in CPME (20 mL, 80 mmol). The mixture was stirred at 0° C. for 1 h, and then concentrated. To a suspension of the residue in THF (60.0 mL) at 0° C. was added MSA (8.52 mL, 53.1 mmol). Upon stirring, the mixture turned into a clear solution. To the solution at 0° C. was added DIPEA (7.95 mL, 45.5 mmol), followed by tert-butyl bromoacetate (2.69 mL, 18.20 mmol) dropwise. The mixture was stirred at 0° C. for 1 h, and then rt for 16 h. The mixture was diluted with EtOAc and a mixture of satd. aq. NH₄Cl (10 mL) and H₂O (20 mL). The pH was adjusted to pH 4 with 2M aq. H₂SO₄. The mixture was extracted twice with EtOAc. The combined organic extracts were washed with brine, dried over Na₂SO₄, filtered, and concentrated. The resulting residue was purified by silica gel flash column chromatography (gradient, CH₂Cl₂/MeOH=1/0 to 80/20) to afford the title compound (5.60 g, 10.76 mmol). ¹H NMR (500 MHz, DMSO-d₆) δ 12.58 (br s, 1H), 7.90 (d, J=7.5 Hz, 2H), 7.72 (d, J=7.5 Hz, 2H), 7.66 (d, J=8.5 Hz, 1H), 7.42 (dd, J=7.5, 7.5 Hz, 2H), 7.33 (dd, J=7.5, 7.5 Hz, 2H), 4.37-4.19 (m, 3H), 4.04-3.85 (m, 1H), 3.20-2.95 (m, 2H), 2.91-2.67 (m, 2H), 2.26-1.96 (m, 2H), 1.72-0.91 (m, 16H). MS (ESI+): m/z 509.4 [M+H].

### Example-2-4

The following compounds were synthesized as outlined for the preparation of Example-4-01 employing appropriate starting materials in the table.

| | Structure and IUPAC name |
|---|---|
| | Fmoc-protected starting material-A |
| | Starting material-B |
| Example | NMR and MS |
| 2-4-02 | 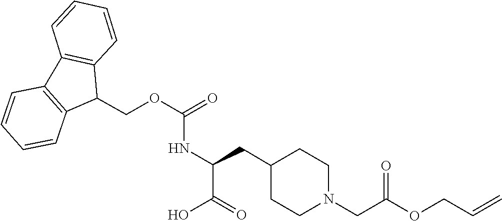 [SMILES: C=CCOC(=O)CN1CCC(C[C@H](NC(=O)OCC2c3ccccc3-c3ccccc32)C(=O)O)CC1] |
| | (2S)-2-({[(9H-fluoren-9-yl)methoxy]carbonyl}amino)-3-{1-[2-oxo-2- |
| | (prop-2-en-1-yloxy)ethyl]piperidin-4-yl}propanoic acid |
| | (2S)-3-{1-[(tert-butoxy)carbonyl]piperidin-4-yl}-2-({[(9H-fluoren-9-yl) |
| | methoxy]carbonyl}amino)propanoic acid (CAS: 204058-25-3) |
| | prop-2-en-1-yl 2-chloroacetate |
| | ¹H NMR (500 MHz, DMSO-d6) δ 12.45 (s, 1H), 7.90 (d, J = 7.5 |
| | Hz, 2H), 7.72 (d, J = 7.5 Hz, 2H), 7.61 (s, 1H), 7.42 (t, J = 7.4 Hz, |
| | 2H), 7.33 (t, J = 7.5 Hz, 2H), 5.95-5.87 (m, 1H), 5.33-5.27 (m, |
| | 1H), 5.23-5.18 (m, 1H), 4.56 (d, J = 5.3 Hz, 2H), 4.34-4.20 (m, |
| | 3H), 4.02-3.93 (m, 1H), 3.22 (s, 2H), 2.84-2.76 (m, 2H), 2.16- |
| | 2.06 (m, 2H), 1.64-1.51 (m, 4H), 1.38-1.26 (m, 1H), 1.24-1.14 |
| | (m, 1H), 1.14-1.04 (1H, m). MS (ESI+): m/z 493.3 [M + H]. |
| 2-4-03 | 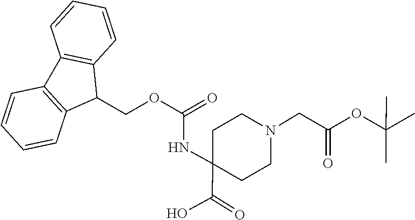 [SMILES: CC(C)(C)OC(=O)CN1CCC(NC(=O)OCC2c3ccccc3-c3ccccc32)(C(=O)O)CC1] |
| | 1-[2-(tert-butoxy)-2-oxoethyl]-4-({[(9H-fluoren-9-yl)methoxy]carbonyl} |
| | amino)piperidine-4-carboxylic acid |
| | 1-[(tert-butoxy)carbonyl]-4-({[(9H-fluoren-9-yl)methoxy]carbonyl}amino) |
| | piperidine-4-carboxylic acid (CAS: 183673-66-7) |
| | tert-butyl bromoacetate |
| | ¹H NMR (300 MHz, DMSO-d6) δ 12.45 (s, 1H), 7.89 (d, J = 7.5 Hz, |
| | 2H), 7.74 (d, J = 7.4 Hz, 2H), 7.54 (s, 1H), 7.47-7.27 (m, 4H), 4.26- |
| | 4.18 (m, 3H), 3.09 (s, 2H), 2.62-2.41 (m, 4H), 2.06-2.79 (m, 4H), |
| | 1.42 (s, 9H). MS (ESI+): m/z 481.2 [M + H]. |
| 2-4-04 | 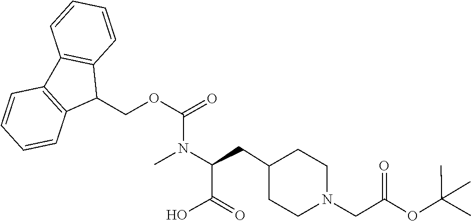 [SMILES: CN(C(=O)OCC1c2ccccc2-c2ccccc21)[C@@H](CC1CCN(CC(=O)OC(C)(C)C)CC1)C(=O)O] |
| | (2S)-3-{1-[2-(tert-butoxy)-2-oxoethyl]piperidin-4-yl}-2-({[(9H-fluoren-9- |
| | yl)methoxy]carbonyl}(methyl)amino)propanoic acid |
| | Intermediate-2-3-08 |
| | tert-butyl bromoacetate |
| | ¹H NMR (500 MHz, DMSO-d6) δ 12.78 (s, 1H), 7.92-7.86 (m, 2H), |
| | 7.67-7.58 (m, 2H), 7.44-7.27 (m, 4H), 4.65-4.23 (m, 4H), 3.50- |
| | 3.01 (m, 2H), 3.00-2.64 (m, 5H), 2.40-2.01 (m, 2H), 1.76-1.38 (m, |
| | 13H), 1.31-0.94 (m, 3H). MS (ESI+): m/z 524.0 [M + H]. |

### Example-2-5-01: (2S)-3-{4-[({4-[2-(tert-butoxy)-2-oxoethyl]piperazine-1-carbonyl}amino)methyl]phenyl}-2-({[(9H-fluoren-9-yl)methoxy]carbonyl}amino)propanoic acid

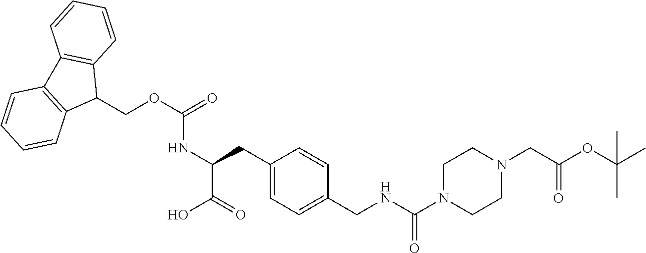 [SMILES: CC(C)(C)OC(=O)CN1CCN(C(=O)NCc2ccc(C[C@H](NC(=O)OCC3c4ccccc4-c4ccccc43)C(=O)O)cc2)CC1]

To a suspension of Zn (31.62 g, 486.49 mmol) in DMF at rt was added I₂ (12.36 g, 48.65 mmol). The mixture was stirred for 10 min. To the mixture at rt was added methyl (2R)-2-{[(9H-fluoren-9-ylmethoxy)carbonyl]amino}-3-iodopropanoate (CAS: 156017-42-4, 87.76 g, 194.59 mmol) The mixture was stirred at rt for 0.5 h. To the mixture were added 1-(4-bromophenyl)methanamine (30 g, 162.162 mmol), Pd₂(dba)₃ (7.43 g, 8.108 mmol), and SPhos (13.33 g, 32.43 mmol). The mixture was stirred at 50° C. for 2 h. The reaction mixture was filtered, and then the filtrate was diluted with EtOAc. The resulting mixture was washed three times with brine, and then dried over Na₂SO₄, filtered, and then concentrated. The resulting residue was triturated with petroleum ether/EtOAc (1/1), and then collected by filtration, which was dissolved in CH₂Cl₂ (460 mL). To the solution at 0° C. were added triphosgene (7.13 g, 24.04 mmol) and DIEA (27.92 mL, 160.28 mmol). The mixture was stirred at 0° C. for 15 min. To the mixture at 0° C. were added NaHCO₃ (17.95 g, 213.70 mmol) in H₂O (230 mL) and tert-butyl 2-(piperazin-1-yl) acetate (21.40 g, 106.85 mmol). The mixture was stirred at rt for 2 h, and then NaH₂PO₄ was added to the mixture. The mixture was extracted three times with CH₂Cl₂. The combined organic extracts were washed three times with brine, and then dried over Na₂SO₄, filtered, and then concentrated. The resulting residue was purified by silica gel flash column chromatography (petroleum ether/EtOAc=50/50). Appropriate fractions were collected and concentrated (28 g). To a mixture of the obtained material in iPrOH (1200 mL) and H₂O (400 mL) at 0° C. were added LiOH·H₂O (7.66 g, 182.70 mmol) and CaCl₂ (81.10 g, 730.84 mmol). The mixture was stirred at rt for 16 h. The reaction was quenched with NaH₂PO₄. The resulting mixture was extracted three times with EtOAc. The combined organic extracts were washed three times with brine, dried over Na₂SO₄, filtrated, and then concentrated. The resulting residue was purified by silica gel flash column chromatography (isocratic, petroleum ether/EtOAc=50/50 then CH₂Cl₂/MeOH=91/9) to afford the title compound (15.05 g, 23.42 mmol). ¹H NMR (400 MHz, DMSO-d₆) δ 12.70 (br s, 1H), 7.88 (d, J=7.6 Hz, 2H), 7.70-7.63 (m, 3H), 7.43-7.38 (m, 2H), 7.35-7.25 (m, 2H), 7.22-7.12 (m, 4H), 7.06-6.96 (m, 1H), 4.25-4.09 (m, 6H), 3.11 (s, 2H), 3.04 (dd, J=13.8, 4.4 Hz, 1H), 2.84 (dd, J=13.8, 10.4 Hz, 1H), 2.48-2.42 (m, 4H), 1.40 (s, 9H). MS (ESI+): m/z 643.2 [M+H].

### Example-2-5

The following compounds were synthesized as outlined for the preparation of Example-2-5-01 employing appropriate starting materials in the table.

| | Structure and IUPAC name |
|---|---|
| | Fmoc-protected starting material-A |
| | Starting material-B |
| Example | NMR and MS |
| 2-5-02 | 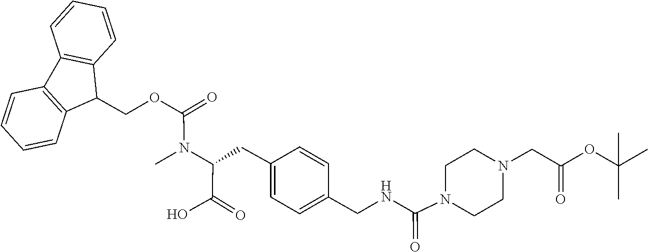 [SMILES: CN(C(=O)OCC1c2ccccc2-c2ccccc21)[C@H](Cc1ccc(CNC(=O)N2CCN(CC(=O)OC(C)(C)C)CC2)cc1)C(=O)O] |
| | (2R)-3-{4-[({4-[2-(tert-butoxy)-2-oxoethyl]piperazine-1-carbonyl}amino) |
| | methyl]phenyl}-2-({[(9H-fluoren-9-yl)methoxy]carbonyl}(methyl)amino)propanoic acid |
| | Reference example-2-2-02 |
| | 1-(4-bromophenyl)methanamine (CAS: 3959-07-7) |
| | ¹H NMR (500 MHz, DMSO-d₆) δ 7.92-7.83 (m, 2H), 7.61-7.49 (m, 2H), |
| | 7.44-7.36 (m, 2H), 7.33-7.25 (m, 2H), 7.16-7.04 (m, 4H), 7.02-6.92 |
| | (m, 1H), 4.68-4.55 (m, 1H), 4.23-4.01 (m, 5H), 3.33-3.18 (m, 6H), 3.10 |
| | (s, 2H), 2.90-2.69 (m, 4H), 2.47-2.38 (m, 4H), 1.41 (s, 9H). MS (ESI+). |
| | MS (ESI+): m/z 657.3 [M + H]. |
| 2-5-03 | 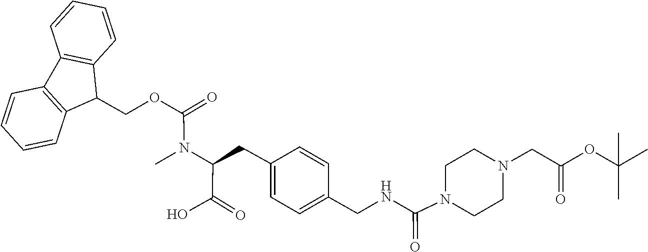 [SMILES: CN(C(=O)OCC1c2ccccc2-c2ccccc21)[C@@H](Cc1ccc(CNC(=O)N2CCN(CC(=O)OC(C)(C)C)CC2)cc1)C(=O)O] |
| | (2S)-3-{4-[({4-[2-(tert-butoxy)-2-oxoethyl]piperazine-1-carbonyl}amino) |
| | methyl]phenyl}-2-({[(9H-fluoren-9-yl)methoxy]carbonyl}(methyl)amino) |
| | propanic acid |
| | Reference example-2-2-01 |
| | 1-(4-bromophenyl)methanamine (CAS: 3959-07-7) |
| | Substantially identical to Example-2-5-02 |

### Reference Example-2-01: methyl (2R)-2-({[(9H-fluoren-9-yl)methoxy]carbonyl}(methyl)amino)-3-iodopropanoate

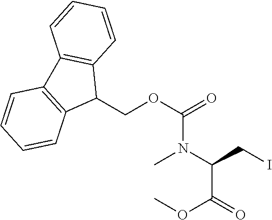 [SMILES: COC(=O)[C@H](CI)N(C)C(=O)OCC1c2ccccc2-c2ccccc21]

To a mixture of methyl (2S)-3-hydroxy-2-(methylamino)propanoate (CAS: 111934-24-8, 64.2 g, 482 mmol) in H₂O (700 mL) and 1,4-dioxane (700 mL) at 0° C. were added Na₂CO₃ (81.0 g, 964 mmol) and Fmoc-OSu (179 g, 531 mmol). The mixture was stirred at rt for 16 h. The reaction was quenched with H₂O. The mixture was extracted three times with EtOAc. The combined organic extracts were dried over Na₂SO₄, filtered, and then concentrated. The resulting residue was purified by silica gel flash column chromatography (petroleum ether/EtOAc=50/50). Appropriate fractions were concentrated (73.8 g, 208 mmol). To a solution of the obtained material (60.0 g, 169 mmol) in CH₂Cl₂ (1.00 L) at 0° C. were added triphenylphosphine (66.4 g, 253 mmol), imidazole (17.2 g, 253 mmol), and iodine (64.3 g, 253 mmol). The mixture was stir red at rt for 0.25 h and then concentrated. The resulting residue was purified by silica gel flash column chromatography (isocratic, petroleum ether/EtOAc=83/17) to afford the title compound (11.0 g, 23.6 mmol). MS (ESI+): m/z 466.2 [M+H].

### Reference Example-2-2

The following compounds were synthesized as outlined for the preparation of Reference Example-2-2-01 employing appropriate starting materials in the table.

| | Structure and IUPAC name |
|---|---|
| | Starting material acid |
| Example | MS data |
| 2-2-02 | 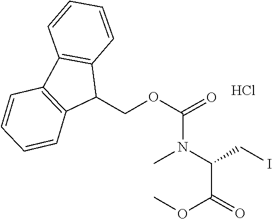 [SMILES: COC(=O)[C@@H](CI)N(C)C(=O)OCC1c2ccccc2-c2ccccc21 ; Cl] |
| | methyl (2S)-2-({[(9H-fluoren-9-yl)methoxy]carbonyl}(methyl) |
| | amino)-3-iodopropanoate |
| | (2R)-3-hydroxy-2-(methylamino)propanoate |
| | (CAS: 1315054-06-8) |
| | Substantially identical to Reference Example-2-2-01 |

### Example-2-6-01: (2R)-3-{3-[(tert-butoxy)carbonyl]phenyl}-2-({[(9H-fluoren-9-yl)methoxy]carbonyl}amino)propanoic acid

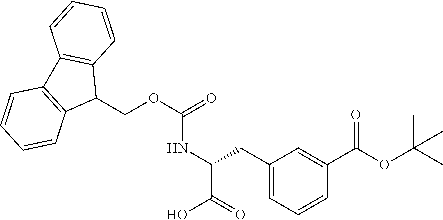 [SMILES: CC(C)(C)OC(=O)c1cccc(C[C@@H](NC(=O)OCC2c3ccccc3-c3ccccc32)C(=O)O)c1]

To a suspension of Zn (3.16 g, 48.4 mmol) in DMF (100 mL) was added I₂ (0.62 g, 2.43 mmol). The mixture was stirred at rt for 10 min. To the mixture at rt was added methyl (2S)-2-[[(9H-fluoren-9-ylmethoxy)carbonyl]amino]-3-iodopropanoate (CAS: 527696-77-1, 7.30 g, 16.2 mmol). The mixture was stirred at rt for an additional 1 h. To the mixture were added tert-butyl 3-bromobenzoate (CAS: 69038-74-0, 4.16 g, 16.2 mmol), Pd₂(dba)₃ CHCl₃ adduct (0.50 g, 0.485 mmol), and SPhos (0.33 g, 0.81 mmol). The mixture was stirred at 50° C. for an additional 12 h, and then filtered. The filtrate was diluted with H₂O. The mixture was extracted three times with EtOAc.

The combined organic extracts were washed with brine, dried over Na₂SO₄, filtered, and then concentrated. The resulting residue was purified by silica gel flash column chromatography (gradient, petroleum ether/EtOAc=99/1 to 75/25). Appropriate fractions were concentrated. To a solution of the resulting residue (5.36 g, 10.7 mmol) in ClCH₂CH₂Cl (50 mL) was added trimethyltin hydroxide (3.89 g, 21.5 mmol). The resulting mixture was stirred at 60° C. for 3 h. The reaction was quenched with aq. citric acid. The mixture was extracted three times with CH₂Cl₂. The combined organic extracts were washed with brine, dried over Na₂SO₄, filtered, and then concentrated. The resulting residue was purified by silica gel flash column chromatography (gradient, petroleum ether/EtOAc=99/1 to 50/50), and then further purified by reversed phase silica gel flash column chromatography (C18 silica gel, gradient, H₂O/CH₃CN=50/50 to 20/80) to afford the title compound (3.20 g, 6.56 mmol). ¹H NMR (400 MHz, Methanol-d₄) δ 7.93-7.76 (m, 4H), 7.61-7.44 (m, 3H), 7.42-7.24 (m, 5H), 4.48 (dd, J=9.6, 4.8 Hz, 1H), 4.31 (dd, J=10.0, 6.8 Hz, 1H), 4.24-4.12 (m, 2H), 3.29 (d, J=4.8 Hz, 1H), 3.04 (dd, J=13.9, 9.7 Hz, 1H), 1.56 (s, 9H); MS (ESI+): m/z 486.1 [M+H].

### Example-2-6-02: (2S)-3-{1-[(tert-butoxy)carbonyl]-1H-pyrrolo[3,2-c]pyridin-3-yl}-2-({[(9H-fluoren-9-yl)methoxy]carbonyl}amino)propanoic acid

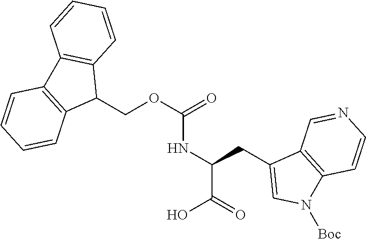 [SMILES: CC(C)(C)OC(=O)n1cc(C[C@H](NC(=O)OCC2c3ccccc3-c3ccccc32)C(=O)O)c2cnccc21]

To a suspension of Zn (18.5 g, 283 mmol) in DMF (800 mL) at rt was added I₂ (3.6 g, 14.18 mmol), and then the mixture was stirred for 10 min. To the mixture at rt was added methyl (2R)-2-{[(9H-fluoren-9-ylmethoxy)carbonyl]amino}-3-iodopropanoate (CAS: 156017-42-4, 64.0 g, 142 mmol). The mixture was stirred at rt for an additional 1 h. To the mixture was added Pd₂(dba)₃ (2.17 g, 2.37 mmol), SPhos (1.94 g, 4.73 mmol), and tert-butyl 3-bromopyrrolo[3,2-c]pyridine-1-carboxylate (CAS: 192189-16-5, 28.0 g, 94.0 mmol). The mixture was stirred at 60° C. for 3 h, and then filtered. The filtrate was diluted with EtOAc. The mixture was washed three times with H₂O, dried over Na₂SO₄, filtered, and then concentrated. The resulting residue was purified by silica gel flash column chromatography (petroleum ether/EtOAc=63/37). Appropriate fractions were concentrated (31.0 g, 57.2 mmol). To a mixture of the obtained material (21.0 g, 38.8 mmol) in 2-propanol (600 mL) and H₂O (200 mL) at 0° C. were added CaCl₂ (69.0 g, 622 mmol) and LiOH·H₂O (6.50 g, 155 mmol). The mixture was stirred at rt for 1 h. The pH of the mixture was adjusted to pH 7 with aq. citric acid. The resulting mixture was extracted three times with EtOAc. The combined organic extracts were washed three times with H₂O, dried over Na₂SO₄, filtered, and then concentrated. The resulting residue was triturated with Et₂O. The obtained solid s ample was purified by reversed phase silica gel flash column chromatography (C18 silica gel, gradient, H₂O/CH₃CN=100/0 to 0/100) to afford the title compound (10.0 g, 19.0 mmol). ¹H NMR (500 MHz, DMSO-d₆) δ 12.90 (br s, 1H), 8.99 (s, 1H), 8.45 (d, J=5.5 Hz, 1H), 7.91-7.86 (m, 4H), 7.62-7.56 (m, 3H), 7.41-7.37 (m, 2H), 7.27-7.21 (m, 2H), 4.39-4.34 (m, 1H), 4.20-4.14 (m, 3H), 3.27 (dd, J=15.0, 4.5 Hz, 1H), 3.12 (dd, J=15.0, 10.5 Hz, 1H), 1.57 (s, 9H); MS (ESI+): m/z 528 [M+H].

### Example-2-6

The following compounds were synthesized as outlined for the preparation of Example-2-6-01 or Example-2-6-02 employing appropriate starting material is in the table.

| | Structurer and IUPAC name |
|---|---|
| | Fmoc-protected starting material-A |
| | Starting material B |
| Example | NMR and MS |
| 2-6-03 | 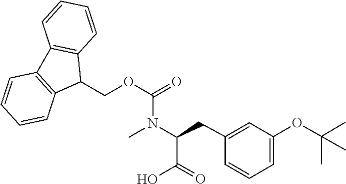 [SMILES: CN(C(=O)OCC1c2ccccc2-c2ccccc21)[C@@H](Cc1cccc(OC(C)(C)C)c1)C(=O)O] |
| | (2S)-3-[3-(tert-butoxy)phenyl]-2-({[(9H-fluoren-9-yl)methoxy] |
| | carbonyl}(methyl)amino)propanoic acid |
| | Reference example-2-2-01 |
| | 1-(tert-butoxy)-3-iodobenzene (CAS: 1429987-72-3) |
| | ¹H NMR (500 MHz, DMSO-d₆) δ 12.92 (br s, 1H), |
| | 7.89 (d, J = 7.5 Hz, 2H), 7.58-7.48 (m, 2H), |
| | 7.44-7.38 (m, 2H), 7.34-7.27 (m, 2H), 7.17-7.14, |
| | (m, 1H), 6.94-6.76 (m, 3H), 4.84-4.74 (m, 1H), |
| | 4.33-4.09 (m, 3H), 3.21-3.10 (m, 1H), 3.05-2.84 |
| | (m, 1H), 2.72-2.69 (m, 3H), 1.24-1.20 (m, 9H). |
| | MS (ESI+): m/z 418.2 [M − tBu + H]. |

### Example-2-7: (2S)-3-{4-[2-({1-[2-(tert-butoxy)-2-oxoethyl]piperidin-4-yl}formamido)ethoxy]phenyl}-2-({[(9H-fluoren-9-yl)methoxy]carbonyl}amino)propanoic acid

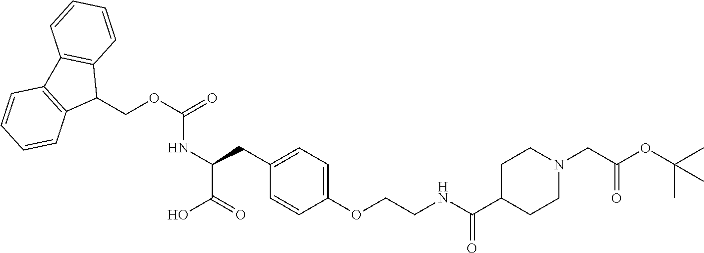 [SMILES: CC(C)(C)OC(=O)CN1CCC(C(=O)NCCOc2ccc(C[C@H](NC(=O)OCC3c4ccccc4-c4ccccc43)C(=O)O)cc2)CC1]

To a suspension of Intermediate-2-3-03 (6.76 g, 14.00 mmol) in THF (140 ml) at 0° C. was added MSA (7.19 mL, 44.8 mmol). The mixture was stirred for 0.5 h at rt, which turned into a clear solution. To the reaction mixture at 0° C. were added DIP EA (4.89 mL, 28.0 mmol) and Intermediate-2-1-01 (5.00 g, 14.70 mmol). The mixture was stirred for 3 h at rt. The mixture was diluted with EtOAc and then pH of the solution was adjusted to 4-5 with 2M aq. NaH₂PO₄ at 0° C. The mixture was extracted twice with EtOAc. The organic extracts were washed with H₂O and brine, dried over Na₂SO₄, filtered, and concentrated. The resulting residue was triturated with hexane. The resulting solid was collected by filtration to afford the title compound (9.63 g, 13.91 mmol). ¹H NMR (500 MHz, DMSO-d6) δ 12.72 (br s, 1H), 8.02-7.99 (m, 1H), 7.88 (2H, d, J=7.55 Hz), 7.71-7.63 (m, 3H), 7.43-7.39 (m, 2H), 7.33-7.27 (m, 2H), 7.17 (2H, d, J=8.50 Hz), 6.88-6.82 (m, 2H), 4.19-4.08 (m, 4H), 3.93-3.90 (m, 2H), 3.39-3.33 (m, 2H), 3.11-2.98 (m, 3H), 2.82-2.77 (m, 3H), 2.18-2.08 (m, 3H), 1.67-1.52 (m, 4H), 1.40 (9H, s). MS (ESI+): m/z 672.4 [M+H].

### Example-2-8: (2S)-3-[4-(2-{[2-(tert-butoxy)-2-oxoethyl][(tert-butoxy)carbonyl]amino}ethoxy)phenyl]-2-({[(9H-fluoren-9-yl)methoxy]carbonyl}amino)propanoic acid

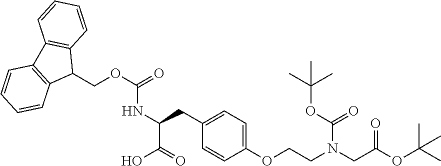 [SMILES: CC(C)(C)OC(=O)CN(CCOc1ccc(C[C@H](NC(=O)OCC2c3ccccc3-c3ccccc32)C(=O)O)cc1)C(=O)OC(C)(C)C]

To a solution of intermediate-2-3-05 (24.0 g, 36.2 mmol) in trifluoroethanol (45 ml) and CH₂Cl₂ (136 mL) at rt was added triethylsilane (23.14 mL, 144.8 mmol) and palladium on carbon (5.78 g, 5.43 mmol). The mixture was stirred at rt for 6 h. The mixture was filtered and the filtrate was concentrated. To a solution of the resulting residue in 1,4-dioxane (145 mL) and H₂O (145 mL) at 0° C. was added NaHCO₃ (12.18 g, 145.0 mmol), followed by a solution of Fmoc-OSu (11.60 g, 34.4 mmol) in 1,4-dioxane (72 mL) dropwise. The mixture was stirred for 3 h at rt, and then pH of the solution was adjusted to 4-5 with 2M aq. HCl at 0° C. The mixture was extracted twice with EtOAc. The organic extracts were washed with brine, dried over Na₂SO₄, filtered, and concentrated. The resulting residue was purified by silica gel flash column chromatography (gradient, CH₂Cl₂/MeOH=100/0 to 85/15) to afford the title compound (21.82 g, 32.7 mmol). 1H NMR (500 MHz, DMSO-d6) δ=12.74 (br s, 1H), 7.88 (d, J=7.5 Hz, 2H), 7.68-7.63 (m, 3H), 7.42-7.39 (m, 2H), 7.33-7.27 (m, 2H), 7.19-7.16 (m, 2H), 6.82-6.80 (m, 2H), 4.21-4.08 (m, 4H), 4.02-3.97 (m, 2H), 3.89-3.88 (m, 2H), 3.57-3.51 (m, 2H), 3.02-2.98 (m, 1H), 2.81-2.79 (m, 1H), 1.36 (s, 18H). MS (ESI+): m/z 661.4 [M+H].

### Example-2-9: (2R)-6-[({1-[2-(tert-butoxy)-2-oxoethyl]azetidin-3-yl}carbamoyl)amino]-2-({[(9H-fluoren-9-yl)methoxy]carbonyl}amino)hexanoic acid

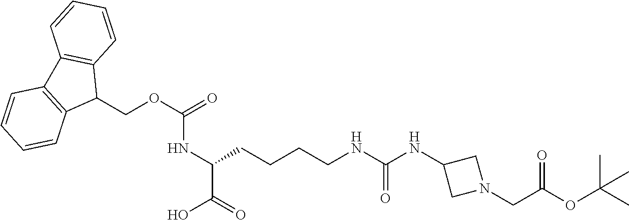 [SMILES: CC(C)(C)OC(=O)CN1CC(NC(=O)NCCCC[C@@H](NC(=O)OCC2c3ccccc3-c3ccccc32)C(=O)O)C1]

To a solution of Intermediate-2-3-06 (1 g, 1.74 mmol) in CH₂Cl₂ (3 mL) and THF (3 mL) at 0° C. were added a solution of Intermediate-2-1-04 (0.36 g, 1.92 mmol) in CH₂Cl₂/THF (1/1, 2.72 mL) and DIPEA (0.37 ml, 2.09 mmol). The mixture was stirred at rt for 20 h, and then quenched with sat. NaHCO₃. The mixture was extracted with EtOAc. The organic extract was washed with 0.5M NaHCO₃ aq. and brine, dried over Na₂SO₄, and concentrated. The residue was purified by silica gel flash column chromatography (gradient, CH₂Cl₂/MeOH=100/0 to 90/10) to give prop-2-en-1-yl (2R)-6-[({1-[2-(tert-butoxy)-2-oxoethyl]azetidin-3-yl}carbamoyl)amino]-2-({[(9H-fluoren-9-yl)methoxy]carbonyl}amino)hexanoate (0.88 g, 1.42 mmol). To a solution of the obtained compound in CH₂Cl₂ (7.1 mL) at 0° C. were added tetrakis(triphenylphosphine)palladium(0) 82 mg, 0.071 mmol) and phenylsilane (307 mg, 2.84 mmol). The mixture was stirred at rt for 1 h, and then concentrated. The residue was purified by silica gel flash column chromatography (gradient, CH₂Cl₂/MeOH=100/0 to 45/55) to give the title compound (0.77 g, 1.20 mmol). ¹H NMR (400 MHz, DMSO-d6) δ 7.89 (d, J=7.5 Hz, 2H), 7.82-7.57 (m, 2H), 7.45-7.25 (m, 4H), 6.25 (d, J=7.8 Hz, 1H), 5.86 (t, J=5.5 Hz, 1H), 5.76 (s, 1H), 4.35-4.19 (m, 2H), 4.19-4.08 (m, 1H), 3.89-3.65 (m, 1H), 3.55-3.47 (m, 2H), 3.07 (s, 2H), 2.98-2.88 (m, 2H), 2.86-2.78 (m, 2H), 1.75-1.50 (m, 2H), 1.43-1.15 (m, 5H), 1.38 (s, 9H). MS (ESI+): m/z 581.5 [M+H].

### Example-2-10: (2R)-6-[({1-[2-(tert-butoxy)-2-oxoethyl]piperidin-4-yl}carbamoyl)oxy]-2-({[(9H-fluoren-9-yl)methoxy]carbonyl}amino)hexanoic acid

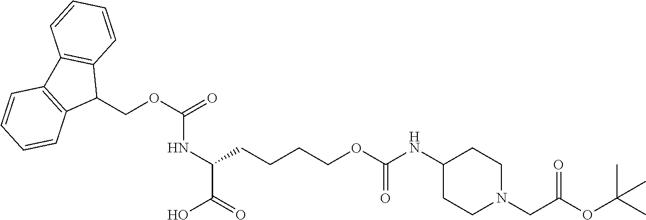 [SMILES: CC(C)(C)OC(=O)CN1CCC(NC(=O)OCCCC[C@@H](NC(=O)OCC2c3ccccc3-c3ccccc32)C(=O)O)CC1]

To a suspension of Intermediate-2-3-07 (886 mg, 1.61 mmol) in Acetonitrile (5.37 mL) at 0° C. were added tert-butyl 4-aminopiperidine-1-carboxylate (CAS: 87120-72-7, 387 mg, 1.93 mmol) and DIPEA (365 μL, 2.09 mmol). The mixture was stirred for 6 h at rt, and then concentrated. The resulting residue was purified by silica gel flash column chromatography (gradient, hexane/EtOAc=100/0 to 80/20) to afford tert-butyl (R)-4-((((5-((((9H-fluoren-9-yl)methoxy)carbonyl)amino)-6-(allyloxy)-6-oxohexyl)oxy)carbonyl)amino)piperidine-1-carboxylate (940 mg, 1.48 mmol). To the solution of a obtained compound (940 mg, 1.48 mmol) in CH₂Cl₂ (4.00 mL) at 0° C. was added 4M HCl in MTBE (2.00 mL). The mixture was stirred for 1 h and then concentrated. To a solution of the residue in DMF (10 mL) at 0° C. were added DIPEA (0.65 mL, 3.70 mmol) and tert-butyl bromoacetate (0.24 mL, 1.63 mmol). The mixture was stirred at rt for 20 h, and then concentrated. EtOAc was added to the residue. The resulting suspension was filtered through celite pad. The filtrate was concentrated. The resulting residue was purified by silica gel flash column chromatography (gradient, hexane/EtOAc=40/60 to 0/100) to afford prop-2-en-1-yl (2R)-6-[({1-[2-(tert-butoxy)-2-oxoethyl]piperidin-4-yl}carbamoyl)oxy]-2-({[(9H-fluoren-9-yl)methoxy]carbonyl}amino)hexanoate (781 mg, 1.20 mmol). To a solution of the obtained compound (777 mg, 1.20 mmol) in CH₂Cl₂ (5.98 mL) at 0° C. were added tetrakis(triphenylphosphine)palladium(0) (34.60 mg, 0.030 mmol) and phenylsilane (294 μL, 2.39 mmol). The mixture was stirred at rt for 1 h, and then concentrated. The resulting residue was purified by silica gel flash column chromatography (gradient, CH2Cl2/MeOH=100/0 to 80/20) to afford the title compound (574 mg, 0.91 mmol). ¹H NMR (500 MHz, DMSO-d6) δ ppm 12.63 (br s, 1H) 7.90 (br d, J=7.48 Hz, 2H) 7.72 (br d, J=7.63 Hz, 2H) 7.56 (br s, 1H) 7.42 (br t, J=7.40 Hz, 2H) 7.33 (br t, J=7.25 Hz, 2H) 7.06 (br d, J=6.56 Hz, 1H) 4.32-4.19 (m, 3H) 3.97-3.83 (m, 3H) 3.26-3.14 (m, 2H) 3.04 (s, 2H) 2.78-2.70 (m, 2H) 2.45-2.14 (m, 3H) 1.74-1.56 (m, 4H) 1.52 (br s, 2H) 1.40 (s, 9H) 1.37-1.33 (m, 2H). MS (ESI+): m/z 610.5 [M+H].

### Example-2-11: (S)-2-((((9H-fluoren-9-yl)methoxy)carbonyl)amino)-3-((2-(tert-butoxy)-2-oxoethyl)(methyl)amino)propanoic acid

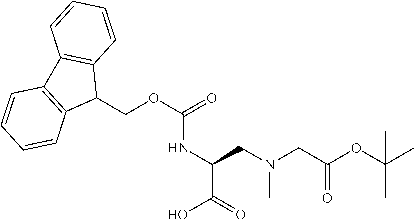 [SMILES: CN(CC(=O)OC(C)(C)C)C[C@H](NC(=O)OCC1c2ccccc2-c2ccccc21)C(=O)O]

To a solution of Intermediate-2-4-01 (5.00 g, 11.22 mmol) in CH₂Cl₂ (28.0 ml) at 0° C. was added TFA (7.00 ml, 91.0 mmol). The mixture was stirred for 7 h at rt, and then dropped into a stirring hexane (120 mL) at 0° C. The resulting suspension was stirred for 1 h at rt. The solid was collected by filtration to afford (4S)-3-{[(9H-fluoren-9-yl)methoxy]carbonyl}-2,2-dioxo-1,2lambda6,3-oxathiazolidine-4-carboxylic acid (4.05 g, 10.4 mmol). To a solution of obtained compound (931 mg, 2.39 mmol) in CH₂Cl₂ (5.2 mL) and MeCN (1.6 mL) at 0° C. were added DIPEA (626 μl, 3.59 mmol) and tert-butyl 2-(methylamino)acetate (CAS: 5616-81-9, 521 mg, 3.59 mmol) in DCM (5.2 mL). The mixture was stirred for 3 h at 0° C., and then quenched with 1M KHSO₄ aq (1 mL) at 0° C. The resulting mixture was stirred for 1 h at 0° C., and then resulting solid was collected by filtration. To the solution of obtained solid in MeOH (30 mL) at rt was added 0.5 M HCl aq (20 mL). The mixture was stirred for 24 h at rt, and then pH was adjusted to 5-7 by satd. NaHCO₃ aq. To the mixture was added EtOAc (60 mL) and water (30 mL). The mixture was extracted three times with EtOAc. The combined organic extracts were washed with brine, dried over Na₂SO₄, and concentrated. The residue was purified by silica gel column chromatography (gradient, CH₂Cl₂/MeOH=97/3-92/8) to afford the title compound (749 mg, 1.648 mmol).

¹H NMR (500 MHz, DMSO-d6) δ 7.90 (d, J=7.5 Hz, 2H), 7.72 (d, J=7.5 Hz, 2H), 7.43-7.31 (m, 5H), 4.30-4.20 (m, 3H), 4.09-4.00 (m, 1H), 3.26-3.22 (m, 2H), 2.92-2.89 (m, 1H), 2.78-2.73 (m, 1H), 2.35-2.30 (m, 3H), 1.41 (s, 9H). MS (ESI+): m/z 455.3 [M+H].

### Example-2-12: (2S)-2-({[(9H-fluoren-9-yl)methoxy]carbonyl}amino)-3-{methyl[2-oxo-2-(prop-2-en-1-yloxy)ethyl]amino}propanoic acid

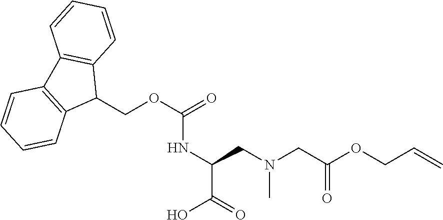 [SMILES: C=CCOC(=O)CN(C)C[C@H](NC(=O)OCC1c2ccccc2-c2ccccc21)C(=O)O]

To a solution of 2-{[(tert-butoxy)carbonyl](methyl)amino}acetic acid (5.00 g, 26.4 mmol) in DMF (80 mL) at rt were added 3-bromoprop-1-ene (3.52 g, 29.1 mmol) and Cs₂CO₃ (9.47 g, 29.1 mmol). The mixture was stirred at 30° C. for 3 h, and then extracted with EtOAc. The organic extract was washed with H₂O, aq. NaHCO₃, and brine, dried over Na₂SO₄, filtered and concentrated. The resulting residue was purified by silica gel flash column chromatography (CH₂Cl₂/MeOH=92/8) to afford prop-2-en-1-yl 2-{[(tert-butoxy)carbonyl](methyl)amino}acetate (3.7 g, 16.1 mmol). To a solution of obtained compound (3.7 g, 16.1 mmol) in CH₂Cl₂ (20 mL) at rt was added TFA (20 mL). The mixture was stirred for 1 h at rt, and then concentrated. To a solution of obtained residue (3.2 g, 13.2 mmol) in MeCN (80 mL) at rt were added Na₂CO₃ (2.79 g, 26.3 mmol) and tert-butyl N-[(3S)-2-oxooxetan-3-yl]carbamate (CAS: 98541-64-1, 2.46 g, 13.16 mmol). The mixture was stirred at rt for 20 h, and then filtered. The filtrate was concentrated. The resulting residue was purified by silica gel flash column chromatography (CH₂Cl₂/MeOH=85/15) to afford (2S)-2-{[(tert-butoxy)carbonyl]amino}-3-{methyl[2-oxo-2-(prop-2-en-1-yloxy)ethyl]amino}propanoic acid (2.15 g, 6.80 mmol). To a solution of obtained compound (2.15 g, 6.80 mmol) in CH₂Cl₂ (15 mL) was added TFA (15 mL). The mixture was stirred for 1 h at rt, and then concentrated. To a solution of obtained residue in dioxane (30 mL) and H₂O (15 mL) at rt were added 2,5-dioxo-1-pyrrolidinyl 9H-fluoren-9-ylmethyl ester-carbonic acid (CAS: 82911-69-1, 3.51 g, 10.4 mmol) and NaHCO₃ (1.17 g, 13.9 mmol). The mixture was stirred at rt for 3 h, and then filtered. The filtrate was acidified with HCl aq., and extracted with EtOAc. The organic extract was washed with brine, dried over Na₂SO₄, filtered and concentrated. The resulting residue was purified by silica gel flash column chromatography (CH₂Cl₂/MeOH=95/5) to afford the title compound (1.04 g, 2.37 mmol). ¹H NMR (500 MHz, Methanol-d4) δ 7.82 (d, J=7.5 Hz, 2H), 7.67 (d, J=7.5 Hz, 2H), 7.45-7.30 (m, 4H), 6.05-5.91 (m, 1H), 5.41-5.20 (m, 2H), 4.71 (d, J=5.8 Hz, 2H), 4.50-4.32 (m, 3H), 4.27 (t, J=6.8 Hz, 1H), 3.91 (s, 2H), 3.45-3.19 (m, 2H), 2.79 (s, 3H); MS (ESI+): m/z 439.1 [M+H].

### Example-2-13: (2S)-4-({4-[2-(tert-butoxy)-2-oxoethyl]piperazine-1-carbonyl}amino)-2-({[(9H-fluoren-9-yl)methoxy]carbonyl}amino)butanoic acid

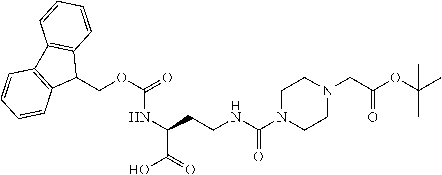 [SMILES: CC(C)(C)OC(=O)CN1CCN(C(=O)NCC[C@H](NC(=O)OCC2c3ccccc3-c3ccccc32)C(=O)O)CC1]

To a solution of tert-butyl 2-(piperazin-1-yl)acetate (CAS: 112257-22-4, 1.21 g, 6.05 mmol) and pyridine (1.22 ml, 15.13 mmol) in CH₂Cl₂ (12.60 ml) at 0° C. was added a solution of diphosgene (CAS: 503-38-8, 1.20 g, 6.05 mmol) in DCM (12.60 ml). The resulting suspension was stirred at 25° C. for 3 h. To a suspension of (S)-2-((((9H-fluoren-9-yl)methoxy)carbonyl)amino)-4-aminobutanoic acid hydrochloride (CAS: 366491-49-8, 1.90 g, 5.04 mmol) in DCM (25.20 ml) at 0° C. were added MSA (CAS: 13435-12-6, 2.32 g, 17.65 mmol) and DIPEA (CAS: 7087-68-5, 4.40 ml, 25.20 mmol). Then to the mixture at 0° C. was added the above reaction mixture dropwise. The resulting mixture was stirred at 23° C. for 20 h, and then quenched with 0.5M NaH₂PO₄ aq. The mixture was extracted twice with DCM. The combined organic extracts were washed with brine, dried over Na₂SO₄, and concentrated. The residue was purified by silica gel column chromatography (gradient, hexane/EtOAc=50/50 to 0/100, then DCM/MeOH=100/0 to 80/20) to afford the title compound (259 mg, 0.43 mmol). ¹H NMR (500 MHz, DMSO-d6) δ 7.89 (d, J=7.5 Hz, 2H), 7.72-7.70 (m, 2H), 7.43-7.32 (m, 4H), 4.26-4.22 (m, 3H), 4.12-4.11 (m, 1H), 3.26-3.23 (m, 4H), 3.18-3.15 (m, 2H), 3.16-2.92 (m, 4H) 2.54 (s, 2H), 2.42-2.37 (m, 4H), 1.84-1.69 (m, 2H), 1.40 (s, 9H). MS (ESI+): m/z 567.4 [M+H].

### Reference Example-3-1-01: methyl (2R)-2-({[(9H-fluoren-9-yl)methoxy]carbonyl}(methyl)amino)-3-iodopropanoate

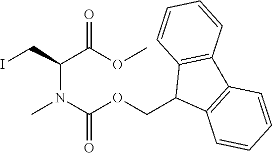 [SMILES: COC(=O)[C@H](CI)N(C)C(=O)OCC1c2ccccc2-c2ccccc21]

To a mixture of methyl (2S)-3-hydroxy-2-(methylamino)propanoate (CAS: 111934-24-8, 64.2 g, 482 mmol) in H₂O (700 mL) and 1,4-dioxane (700 mL) at 0° C. were added Na₂CO₃ (81.0 g, 964 mmol) and Fmoc-OSu (179 g, 531 mmol). The mixture was stirred at rt for 16 h. The reaction was quenched with H₂O. The mixture was extracted three times with EtOAc. The combined organic extracts were dried over Na₂SO₄, filtered, and then concentrated. The resulting residue was purified by silica gel flash column chromatography (petroleum ether/EtOAc=50/50). Appropriate fractions were concentrated (73.8 g, 208 mmol). To a solution of the obtained material (60.0 g, 169 mmol) in CH₂Cl₂ (1.00 L) at 0° C. were added triphenylphosphine (66.4 g, 253 mmol), imidazole (17.2 g, 253 mmol), and iodine (64.3 g, 253 mmol). The mixture was stirred at rt for 0.25 h and then concentrated. The resulting residue was purified by silica gel flash column chromatography (isocratic, petroleum ether/EtOAc=83/17) to afford the title compound (11.0 g, 23.6 mmol). LC-MS (ESI, m/z): [M+H]⁺ 466.

The following compounds were synthesized as outlined for the preparation of Reference Example-3-1-01 employing appropriate starting materials in the table.

| | Structure and IUPAC name |
|---|---|
| Reference | Starting material acid |
| Example | MS data |
| 1-02 | 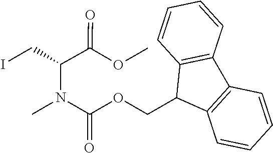 [SMILES: COC(=O)[C@@H](CI)N(C)C(=O)OCC1c2ccccc2-c2ccccc21] |
| | methyl (2S)-2-({[(9H-fluoren-9-yl)methoxy] |
| | carbonyl}(methyl)amino)-3-iodopropanoate |
| | methyl (2R)-3-hydroxy-2-(methylamino) |
| | propanoate (CAS: 1315054-06-8) |
| | Substantially identical to Reference Example-3-1-01 |

### Reference Example-3-1-03: methyl (2S)-2-({[(9H-fluoren-9-yl)methoxy]carbonyl}amino)-4-iodobutanoate

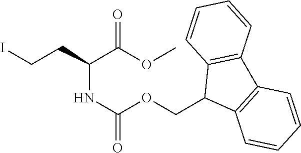 [SMILES: COC(=O)[C@H](CCI)NC(=O)OCC1c2ccccc2-c2ccccc21]

To a solution of methyl (2S)-2-({[(9H-fluoren-9-yl)methoxy]carbonyl}amino)-4-hydroxybutanoate (CAS: 1028055-26-6, 1.00 g, 2.80 mmol) in CH₂Cl₂ (30.0 mL) at 0° C. were added triphenylphosphine (1.10 g, 4.19 mmol), imidazole (0.29 g, 4.22 mmol), and iodine (1.07 g, 4.22 mmol). The mixture was stirred at rt for 0.5 h and then concentrated. The resulting residue was purified by silica gel flash column chromatography (isocratic, petroleum ether/EtOAc=91/9) to afford the title compound (1.00 g, 2.15 mmol). LC-MS (ESI, m/z): [M+H]⁺ 466.

### Reference Example-3-1-04: (2R)-3-{3-[(tert-butoxy)carbonyl]phenyl}-2-({[(9H-fluoren-9-yl)methoxy]carbonyl}amino)propanoic acid

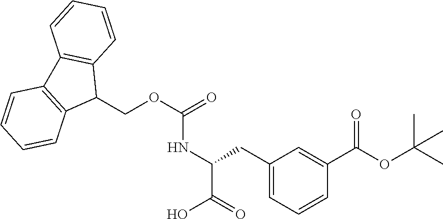 [SMILES: CC(C)(C)OC(=O)c1cccc(C[C@@H](NC(=O)OCC2c3ccccc3-c3ccccc32)C(=O)O)c1]

To a suspension of Zn (3.16 g, 48.4 mmol) in DMF (100 mL) was added I₂ (0.62 g, 2.43 mmol). The mixture was stirred at rt for 10 min. To the mixture at rt was added methyl (2S)-2-[[(9H-fluoren-9-ylmethoxy)carbonyl]amino]-3-iodopropanoate (CAS: 527696-77-1, 7.30 g, 16.2 mmol). The mixture was stirred at rt for an additional 1 h. To the mixture were added tert-butyl 3-bromobenzoate (CAS: 69038-74-0, 4.16 g, 16.2 mmol), Pd₂(dba)₃ CHCl₃ adduct (0.50 g, 0.485 mmol), and SPhos (0.33 g, 0.81 mmol). The mixture was stirred at 50° C. for an additional 12 h, and then filtered. The filtrate was diluted with H₂O. The mixture was extracted three times with EtOAc. The combined organic extracts were washed with brine, dried over Na₂SO₄, filtered, and then concentrated. The resulting residue was purified by silica gel flash column chromatography (gradient, petroleum ether/EtOAc=99/1 to 75/25). Appropriate fractions were concentrated. To a solution of the resulting residue (5.36 g, 10.7 mmol) in ClCH₂CH₂Cl (50 mL) was added trimethyltin hydroxide (3.89 g, 21.5 mmol). The resulting mixture was stirred at 60° C. for 3 h. The reaction was quenched with aq. citric acid. The mixture was extracted three times with CH₂Cl₂. The combined organic extracts were washed with brine, dried over Na₂SO₄, filtered, and then concentrated. The resulting residue was purified by silica gel flash column chromatography (gradient, petroleum ether/EtOAc=99/1 to 50/50), and then further purified by reversed phase silica gel flash column chromatography (C18 silica gel, gradient, H₂O/CH₃CN=50/50 to 20/80) to afford the title compound (3.20 g, 6.56 mmol). ¹H NMR (400 MHz, Methanol-d₄) δ 7.93-7.76 (m, 4H), 7.61-7.44 (m, 3H), 7.42-7.24 (m, 5H), 4.48 (dd, J=9.6, 4.8 Hz, 1H), 4.31 (dd, J=10.0, 6.8 Hz, 1H), 4.24-4.12 (m, 2H), 3.29 (d, J=4.8 Hz, 1H), 3.04 (dd, J=13.9, 9.7 Hz, 1H), 1.56 (s, 9H); MS (ESI+): m/z 486.1 [M+H]

### Reference Example-3-2-01: 4-tert-butyl 3-(9H-fluoren-9-yl)methyl (4S)-2,2-dioxo-1,2lambda6,3-oxathiazolidine-3,4-dicarboxylate

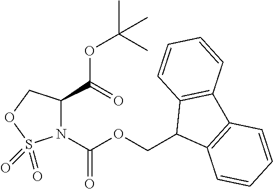 [SMILES: CC(C)(C)OC(=O)[C@@H]1COS(=O)(=O)N1C(=O)OCC1c2ccccc2-c2ccccc21]

To a solution of thionyl chloride (0.474 ml, 6.50 mmol) and MeCN (15 ml) at −40° C. was added (2S)-2-({[(9H-fluoren-9-yl)methoxy]carbonyl}amino)-3-hydroxypropanoic acid (CAS: 110797-35-8, 1.92 g, 5 mmol) in MeCN (10 ml) dropwise followed by pyridine (2.02 ml, 25 mmol) while maintaining the temperature below −30° C. The mixture was stirred at rt for 1 h, and then quenched with water. The mixture was extracted with EtOAc. The organic extract was washed with H₂O, dried over Na₂SO₄, filtered, and concentrated. To a solution of obtained residue in MeCN (30 mL) at 0° C. was added ruthenium(III) chloride (CAS: 10049-08-8, 21.0 mg, 0.100 mmol), sodium periodate (CAS: 7790-28-5, 12.8 g, 60.0 mmol) and H₂O (100 ml). The mixture was stirred at 0° C. for 2 h, and then concentrated. The mixture was extracted with Et₂O. The organic extract was washed with NaHCO₃ aq. and brine, dried over Na₂SO₄, filtered, and concentrated. The residue was purified by silica gel flash column chromatography (gradient, hexane/EtOAc=100/0 to 0/100) to afford the title compound (1.36 g, 3.05 mmol). LC-MS (ESI, m/z): [M+Na]+468.

### Reference Example-3-2-02: (4S)-3-{[(9H-fluoren-9-yl)methoxy]carbonyl}-2,2-dioxo-1,2lambda6,3-oxathiazolidine-4-carboxylic acid

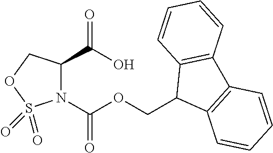 [SMILES: O=C(O)[C@@H]1COS(=O)(=O)N1C(=O)OCC1c2ccccc2-c2ccccc21]

To a solution of Reference example-3-2-01 (5.00 g, 11.22 mmol) in CH₂Cl₂ (28.0 ml) at 0° C. was added TFA (7.00 ml, 91.0 mmol). The mixture was stirred at rt for 7 h, and then dropped into a stirring hexane (50 mL) at 0° C. The resulting suspension was stirred at rt for 1 h. The resulting solid was collected by filtration to afford the title compound (4.05 g, 10.40 mmol). LC-MS (ESI, m/z): [M+H]⁺ 389.

### Reference Example-3-2-03 3-((9H-fluoren-9-yl)methyl) 4-(tert-butyl) (S)-1,2,3-oxathiazinane-3,4-dicarboxylate 2,2-dioxide

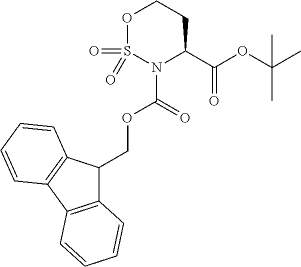 [SMILES: CC(C)(C)OC(=O)[C@@H]1CCOS(=O)(=O)N1C(=O)OCC1c2ccccc2-c2ccccc21]

To a mixture of (S)-3-((((9H-fluoren-9-yl)methoxy)carbonyl)amino)-4-(tert-butoxy)-4-oxobutanoic acid (CAS: 129460-09-9, 15 g, 36.5 mmol) in THF (350 mL) at −10° C. were added N-methylmorpholine (CAS: 109-02-4, 5.21 mL, 47.4 mmol) and ethyl chloroformate (CAS: 541-41-3, 3.85 mL, 40.1 mmol). The resulting mixture was stirred at −10° C. for 30 min. NaBH₄ (CAS: 16940-66-2, 1.38 g, 36.5 mmol) was added and the resulting mixture was stirred at −10° C. for 30 min. A freshly prepared solution of NaBH₄ (2.76 g, 72.9 mmol) in water (80 mL) was slowly added dropwise over 15 min. The resulting mixture was stirred at −10° C. for 30 min and then diluted with EtOAc and quenched with 1M aq. KHSO₄. The resulting mixture was extracted with EtOAc. The combined organic extracts were washed with satd. aq. NH₄Cl and brine, dried over Na₂SO₄, filtered, and concentrated. To a mixture of imidazole (11.68 g, 172 mmol) and Et₃N (13.33 mL, 96 mmol) in DCM (300 mL) at −40° C. were added thionyl chloride (3.41 mL, 46.7 mmol) and a solution of the obtained material (14.51 g, 36.5 mmol) in DCM (100 mL). The resulting mixture was allowed to slowly warm up to 0° C. and stirred at 0° C. for 6 h. The mixture was poured into water. The resulting mixture was extracted twice with DCM. The combined organic extracts were washed with brine, dried over Na₂SO₄, filtered, and then concentrated. The resulting residue was purified by flash column chromatography (gradient, heptane/EtOAc=95/5 to 70/30). Appropriate fractions were concentrated. To a solution of the obtained material (9.7 g, 21.87 mmol) in MeCN (200 mL) and Water (50 mL) at 0° C. were added ruthenium(III) chloride (CAS: 14898-67-0, 0.023 g, 0.109 mmol) and then sodium periodate (CAS: 7790-28-5, 7.48 g, 35.0 mmol) in Water (150 mL). The resulting mixture was stirred at 0° C. for 1 h. The resulting mixture was allowed to slowly warm up to 25° C. and stirred at 25° C. for ca. 16 h. A solution of ruthenium(III) chloride (0.068 g, 0.328 mmol) and sodium periodate (4.68 g, 21.87 mmol) in water (50 mL) was added at 0° C. The resulting mixture was allowed to slowly warm to 25° C. and stirred at 25° C. for 4 h. The mixture was poured into water. The resulting mixture was extracted with EtOAc. The combined organic extracts were washed with brine, dried over Na₂SO₄, filtered, and then concentrated. The resulting residue was purified by flash column chromatography (gradient, heptane/EtOAc=90/10 to 70/30) to afford the title compound (2.25 g, 4.90 mmol). ¹H NMR (500 MHz, CDCl₃) δ 7.78-7.72 (m, 4H), 7.42 (m, 2H), 7.34 (m, 2H), 5.18 (m, 1H), 4.81 (m, 1H), 4.71-4.67 (m, 1H), 4.54-4.51 (m, 2H), 4.38-4.35 (m, 1H), 2.68-2.63 (m, 1H), 2.48-2.41 (m, 1H), 1.46 (s, 9H). LC-MS (ESI, m/z): [M+H₂O+H]⁺ 477.3.

### Reference Example-3-3-01: (S)-2-((((9H-fluoren-9-yl)methoxy)carbonyl)amino)-3-((2-(tert-butoxy)-2-oxoethyl)(methyl)amino)propanoic acid

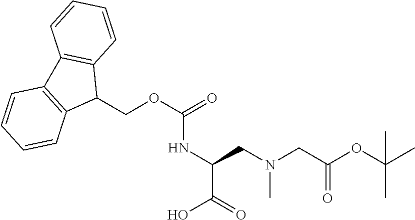 [SMILES: CN(CC(=O)OC(C)(C)C)C[C@H](NC(=O)OCC1c2ccccc2-c2ccccc21)C(=O)O]

To a solution of Reference example-3-2-01 (5.00 g, 11.22 mmol) in CH₂Cl₂ (28.0 ml) at 0° C. was added TFA (7.00 ml, 91.0 mmol). The mixture was stirred for 7 h at rt, and then dropped into a stirring hexane (120 mL) at 0° C. The resulting suspension was stirred for 1 h at rt. The solid was collected by filtration to afford (4S)-3-{[(9H-fluoren-9-yl)methoxy]carbonyl}-2,2-dioxo-1,2lambda6,3-oxathiazolidine-4-carboxylic acid (4.05 g, 10.4 mmol). To a solution of obtained compound (931 mg, 2.39 mmol) in CH₂Cl₂ (5.2 mL) and MeCN (1.6 mL) at 0° C. were added DIPEA (626 μl, 3.59 mmol) and tert-butyl 2-(methylamino)acetate (CAS: 5616-81-9, 521 mg, 3.59 mmol) in DCM (5.2 mL). The mixture was stirred for 3 h at 0° C., and then quenched with 1M KHSO₄ aq (1 mL) at 0° C. The resulting mixture was stirred for 1 h at 0° C., and then resulting solid was collected by filtration. To the solution of obtained solid in MeOH (30 mL) at rt was added 0.5 M HCl aq (20 mL). The mixture was stirred for 24 h at rt, and then pH was adjusted to 5-7 by satd. NaHCO₃ aq. To the mixture was added EtOAc (60 mL) and water (30 mL). The mixture was extracted three times with EtOAc. The combined organic extracts were washed with brine, dried over Na₂SO₄, and concentrated. The residue was purified by silica gel column chromatography (gradient, CH₂Cl₂/MeOH=97/3-92/8) to afford the title compound (749 mg, 1.648 mmol). ¹H NMR (500 MHz, DMSO-d₆) δ 7.90 (d, J=7.5 Hz, 2H), 7.72 (d, J=7.5 Hz, 2H), 7.43-7.31 (m, 5H), 4.30-4.20 (m, 3H), 4.09-4.00 (m, 1H), 3.26-3.22 (m, 2H), 2.92-2.89 (m, 1H), 2.78-2.73 (m, 1H), 2.35-2.30 (m, 3H), 1.41 (s, 9H). MS (ESI+): m/z 455.3 [M+H].

### Reference Example-3-4-01: (2S)-3-{1-[2-(tert-butoxy)-2-oxoethyl]piperidin-4-yl}-2-({[(9H-fluoren-9-yl)methoxy]carbonyl}amino)propanoic acid

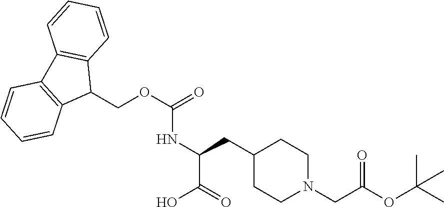 [SMILES: CC(C)(C)OC(=O)CN1CCC(C[C@H](NC(=O)OCC2c3ccccc3-c3ccccc32)C(=O)O)CC1]

To a solution of (S)-2-((((9H-fluoren-9-yl)methoxy)carbonyl)amino)-3-(1-(tert-butoxycarbonyl)piperidin-4-yl)propanoic acid (7.5 g, 15.16 mmol) in CH₂Cl₂ (60 mL) at 0° C. was added 4M hydrogen chloride in CPME (20 mL, 80 mmol). The mixture was stirred at 0° C. for 1 h, and then concentrated. To a suspension of the residue in THF (60.0 mL) at 0° C. was added MSA (8.52 mL, 53.1 mmol). Upon stirring, the mixture turned into a clear solution. To the solution at 0° C. was added DIPEA (7.95 mL, 45.5 mmol), followed by tert-butyl bromoacetate (2.69 mL, 18.20 mmol) dropwise. The mixture was stirred at 0° C. for 1 h, and then rt for 16 h. The mixture was diluted with EtOAc and a mixture of satd. aq. NH₄Cl (10 mL) and H₂O (20 mL). The pH was adjusted to pH 4 with 2M aq. H₂SO₄. The mixture was extracted twice with EtOAc. The combined organic extracts were washed with brine, dried over Na₂SO₄, filtered, and concentrated. The resulting residue was purified by silica gel flash column chromatography (gradient, CH₂Cl₂/MeOH=1/0 to 80/20) to afford the title compound (5.60 g, 10.76 mmol). ¹H NMR (500 MHz, DMSO-d₆) δ 12.58 (br s, 1H), 7.90 (d, J=7.5 Hz, 2H), 7.72 (d, J=7.5 Hz, 2H), 7.66 (d, J=8.5 Hz, 1H), 7.42 (dd, J=7.5, 7.5 Hz, 2H), 7.33 (dd, J=7.5, 7.5 Hz, 2H), 4.37-4.19 (m, 3H), 4.04-3.85 (m, 1H), 3.20-2.95 (m, 2H), 2.91-2.67 (m, 2H), 2.26-1.96 (m, 2H), 1.72-0.91 (m, 16H). MS (ESI+): m/z 509.4 [M+H].

### Reference Example-3-5-01: (2S)-3-[4-(2-{4-[2-(tert-butoxy)-2-oxoethyl]piperazin-1-yl}-2-oxoethoxy)phenyl]-2-({[(9H-fluoren-9-yl)methoxy]carbonyl}amino)propanoic acid

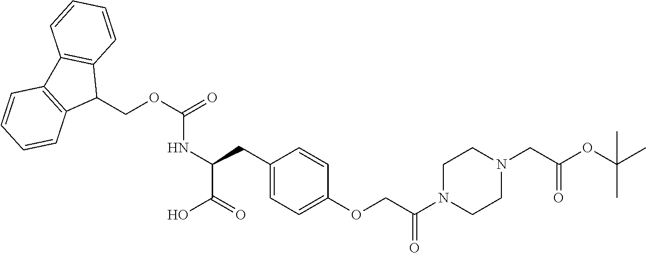 [SMILES: CC(C)(C)OC(=O)CN1CCN(C(=O)COc2ccc(C[C@H](NC(=O)OCC3c4ccccc4-c4ccccc43)C(=O)O)cc2)CC1]

To a solution of (2S)-3-{4-[2-(tert-butoxy)-2-oxoethoxy]phenyl}-2-({[(9H-fluoren-9-yl)methoxy]carbonyl}amino)propanoic acid (CAS: 181951-92-8, 40 g, 77.28 mmol) in DMF (400 mL) at 0° C. were added NaHCO₃ (13.00 g, 154.57 mmol) and 3-bromoprop-1-ene (11.20 g, 92.74 mmol). The mixture was stirred at rt for 2 h, and then diluted with H₂O. The mixture was extracted twice with EtOAc. The combined organic extracts were washed three times with brine, dried over Na₂SO₄, filtrated, and then concentrated. The resulting residue was purified by silica gel column chromatography (petroleum ether/EtOAc=50/50). Appropriate fractions were concentrated. To a solution of the resulting residue (31 g, 55.60 mmol) in CH₂Cl₂ (300 mL) at 0° C. was added TFA (150 mL). The mixture was stirred at rt for 2 h, and then concentrated. The resulting residue was suspended in toluene, and then concentrated. To a solution of the resulting residue in DMF (400 mL) at rt were added tert-butyl 2-(piperazin-1-yl)acetate (13.609 g, 67.983 mmol), HATU (47.00 g, 123.63 mmol), and DIPEA (31.93 g, 247.26 mmol). The mixture was stirred at rt for 3 h, and then diluted with H₂O. The mixture was extracted three times with EtOAc. The combined organic extracts were washed with three times with brine, dried over Na₂SO₄, filtered, and then concentrated. The resulting residue was purified by silica gel flash column chromatography (petroleum ether/EtOAc=50/50). Appropriate fractions were concentrated. To a solution of the resulting residue (23 g, 33.64 mmol) in THF at rt were added morpholine (5.86 g, 67.26 mmol) and Pd(PPh₃)₄ (3.89 g, 3.36 mmol). The mixture was stirred at rt for 0.5 h. The reaction was quenched with H₂O. The pH of the mixture was adjusted to 6 with citric acid. The mixture was extracted three times with EtOAc. The combined organic extracts were washed three times with brine, dried over Na₂SO₄, filtrated, and then concentrated. The resulting residue was purified by reverse-phase flash chromatography (Column, C18 silica gel; gradient, CH₃CN/H₂O=10/90 to 50/50) to afford the title compound (11.0 g, 17.1 mmol). ¹H NMR (400 MHz, DMSO-d₆) δ 12.68 (br s, 1H), 7.88 (d, J=7.2 Hz, 2H), 7.73-7.37 (m, 8H), 7.36-7.24 (m, 2H), 7.17 (d, J=8.4 Hz, 2H), 6.90-6.75 (m, 2H), 4.73 (s, 2H), 4.25-4.06 (m, 4H), 3.61 (br s, 1H), 3.43 (br s, 4H), 3.14 (s, 2H), 3.01 (dd, J=13.8, 4.0 Hz, 1H), 2.80 (dd, J=13.8, 10.4 Hz, 1H), 1.40 (s, 9H). MS (ESI+): m/z 644.2 [M+H].

### Reference Example-3-6-01: (2S)-3-(2H-1,3-benzodioxol-5-yl)-2-({[(9H-fluoren-9-yl)methoxy]carbonyl}(methyl)amino)propanoic acid

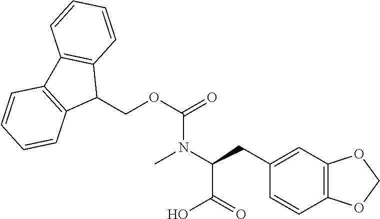 [SMILES: CN(C(=O)OCC1c2ccccc2-c2ccccc21)[C@@H](Cc1ccc2c(c1)OCO2)C(=O)O]

To a solution of (2S)-3-(2H-1,3-benzodioxol-5-yl)-2-[(tert-butoxycarbonyl)amino]propanoic acid (CAS: 209525-79-1, 32.0 g, 103 mmol) in THF (300 mL) at 0° C. was added sodium hydride (12.4 g, 60% dispersion in mineral oil) portionwise, followed by iodomethane (44.1 g, 310 mmol) dropwise. The mixture was stirred at rt for 24 h. The reaction was quenched with H₂O. The pH of the mixture was adjusted to 5 with citric acid. The resulting mixture was extracted three times with EtOAc. The combined extracts were washed three times with brine, dried over Na₂SO₄, filtered, and then concentrated. The resulting residue was purified by silica gel flash column chromatography (gradient, petroleum ether/EtOAc=50/50). Appropriate fractions were concentrated. The resulting residue (27.0 g, 83.5 mmol) and 2M HCl in 1,4-dioxane (270 mL) was stirred at rt for 1 h. The resulting mixture was concentrated. To the resulting residue in (13.4 g, 51.6 mmol) in 1,4-dioxane (300 mL) and H₂O (100 mL) at 0° C. was added NaHCO₃ (8.67 g, 103 mmol), followed by Fmoc-OSu (17.4 g, 51.6 mmol) portionwise. The mixture was stirred at rt for 8 h. The pH of the solution was adjusted to 5 with citric acid. The mixture was extracted three times with EtOAc. The combined extracts were washed three times with brine, dried over Na₂SO₄, filtered, and then concentrated. The resulting residue was purified by reversed phase silica gel flash column chromatography (C18 silica gel, gradient, H₂O/CH₃CN with 0.1% NH₄HCO₃=60/40 to 30/70) to afford the title compound (5.01 g, 11.3 mmol). ¹H NMR (500 MHz, DMSO-d₆) δ 7.88 (dd, J=7.0, 6.0 Hz, 2H), 7.55 (d, J=7.5 Hz, 1H), 7.47 (dd, J=14.5, 7.5 Hz, 1H), 7.40 (dd, J=7.5, 7.0 Hz, 2H), 7.32-7.26 (m, 2H), 6.76 (dd, J=16.5, 8.0 Hz, 2H), 6.66-6.59 (m, 1H), 5.92-5.83 (m, 2H), 4.64-4.54 (m, 1H), 4.25-4.04 (m, 3H), 3.19-3.13 (m, 1H), 2.84-2.73 (m, 4H); MS (ESI+): m/z 446.2 [M+H].

### Example-3-1-01: (2S)-2-({[(9H-fluoren-9-yl)methoxy]carbonyl}(methyl)amino)-6-(N-methylacetamido)hexanoic acid

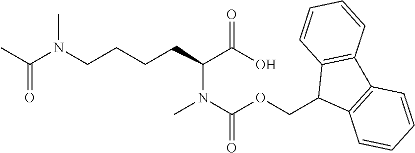 [SMILES: CC(=O)N(C)CCCC[C@@H](C(=O)O)N(C)C(=O)OCC1c2ccccc2-c2ccccc21]

To a solution of (2S)-2-{[(benzyloxy)carbonyl]amino}-6-{[(tert-butoxy)carbonyl]amino}hexanoic acid (CAS: 2389-60-8, 10.0 g, 26.3 mmol) in THF (131 mL) at 0° C. was added sodium hydride (3.68 g, 60% dispersion in mineral oil, 92.0 mmol). The mixture was stirred at 0° C. for 0.5 h. To the mixture was added iodomethane (7.65 g, 53.9 mmol) dropwise. The mixture was stirred at rt for 12 h, and then quenched with 2M aq. H₂SO₄ at 0° C. The mixture was extracted twice with EtOAc. The organic extract was washed with brine, dried over Na₂SO₄, filtered, and then concentrated. The resulting residue was purified by reversed phase silica gel flash column chromatography (C18 silica gel, gradient, H₂O/CH₃CN=100/0 to 40/60). Appropriate fractions were concentrated. To a solution of the resulting residue (4.49 g, 11.0 mmol) in MeOH (22.0 ml) at rt was added palladium on carbon (1.17 g, 1.10 mmol) and ammonium formate (2.77 g, 44.0 mmol). The mixture was stirred at 60° C. for 2 h, and then filtered. The filtrate was concentrated. To a solution of the resulting residue (3.02 g, 11.0 mmol) in 1,4-dioxane (27.5 mL) and H₂O (27.5 mL) at rt was added NaHCO₃ (2.33 g, 22.0 mmol), followed by Fmoc-OSu (4.45 g, 13.2 mmol). The mixture was stirred at rt overnight, and then the volatiles were removed under reduced pressure and a pH of the solution was adjusted to 3 with 2M aq. HCl at 0° C. The mixture was extracted twice with CH₂Cl₂. The organic extracts were washed with water and brine, dried over Na₂SO₄, filtered, and concentrated. The resulting residue was purified by reversed phase silica gel flash column chromatography (C18 silica gel, gradient, H₂O/CH₃CN=100/0 to 40/60). Appropriate fractions were concentrated. The mixture of the resulting residue (5.56 g, 11.2 mmol) and 4M hydrogen chloride in CPME (56.0 mL, 224 mmol) was stirred at rt for 2 h, and then concentrated. To a solution of the resulting residue (4.85 g, 11.2 mmol) in THF (56.0 mL) and H₂O (56.0 mL) at 0° C. was added NaHCO₃ (7.53 g, 90.0 mmol), followed by acetic anhydride (2.29 g, 22.4 mmol). The mixture was stirred at rt for 2 h, and then pH of the solution was adjusted to 1 with 1M aq. HCl at 0° C. The mixture was extracted with EtOAc. The organic extracts were concentrated. The resulting residue was purified by silica gel flash column chromatography (isocratic, CH₂Cl₂/MeOH=91/9) to afford the title compound (4.85 g, 10.8 mmol). ¹H NMR (500 MHz, DMSO-d₆) δ 12.75 (br, 1H), 7.91-7.88 (m, 2H), 7.67-7.61 (m, 2H), 7.44-7.15 (m, 4H), 4.54-4.25 (m, 4H), 3.27-3.15 (m, 2H), 2.89-2.70 (m, 5H), 1.96-0.98 (m, 10H). LC-MS (ESI, m/z): [M+H]⁺ 439.

### Example-3-1-02: 2-({[(9H-fluoren-9-yl)methoxy]carbonyl}[4-(N-methylmethanesulfonamido)butyl]amino)acetic acid

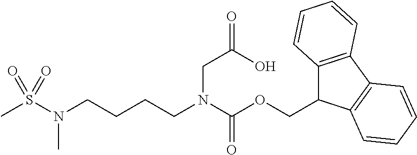 [SMILES: CN(CCCCN(CC(=O)O)C(=O)OCC1c2ccccc2-c2ccccc21)S(C)(=O)=O]

To a solution of tert-butyl N-(4-aminobutyl)-N-methylcarbamate (CAS: 144222-23-1, 15.0 g, 74.1 mmol) in CH₃CN (225 mL) at 0° C. was added tert-butyl 2-bromoacetate (CAS: 5292-43-3, 21.7 g, 111.2 mmol) and DIPEA (19.2 g, 148.3 mmol). The mixture was stirred at rt overnight. The mixture was extracted with EtOAc. The combined organic extracts were washed with brine, dried over Na₂SO₄, filtrated, and concentrated. The resulting residue was purified by silica gel flash column chromatography (isocratic, petroleum ether/EtOAc=70/30). Appropriate fractions were concentrated. To a solution of the obtained material (14.0 g, 44.2 mmol) in 1,4-dioxane (280 mL) at 0° C. was added H₂O (280 mL), and NaHCO₃ (7.43 g, 88.5 mmol), followed by Fmoc-Cl (CAS: 28920-43-6 13.7 g, 53.1 mmol). The mixture was stirred at rt overnight. The pH of the mixture was adjusted to 5 with aq. citric acid. The mixture was extracted three times with EtOAc. The organic extracts were washed with brine, dried over Na₂SO₄, filtered, and concentrated. The resulting residue was purified by silica gel flash column chromatography (isocratic, petroleum ether/EtOAc=50/50). Appropriate fractions were concentrated. The mixture of the resulting residue (17.0 g, 31.6 mmol) and 12M aq. HCl in 1,4-dioxane (340 mL) was stirred at rt for 2 h, and then concentrated. To a solution of the resulting residue (9.00 g, 23.5 mmol) in THF (270 mL) at 0° C. was added BSA (23.9 g, 117.6 mmol). The mixture was stirred at rt overnight. To the mixture at 0° C. was added DIPEA (12.3 mL, 70.6 mmol) and methanesulfonyl chloride (3.28 mL, 42.4 mmol). The mixture was stirred at rt for 2 h. The mixture was washed three times with brine. The resulting solution was extracted with EtOAc. The organic extracts were dried over Na₂SO₄, filtered, and concentrated. The resulting residue was purified by reversed phase silica gel flash column chromatography (C18 silica gel, gradient, H₂O/CH₃CN=95/5 to 0/100) to afford the title compound (3.25 g, 7.05 mmol). ¹H NMR (400 MHz, DMSO-d₆) δ 12.66 (br, 1H), 7.92-7.87 (m, 2H), 7.69-7.60 (m, 2H), 7.43-7.39 (m, 2H), 7.38-7.29 (m, 2H), 4.48 (d, J=5.4 Hz, 1H), 4.33-4.18 (m, 2H), 3.98-3.76 (m, 2H), 3.29-3.20 (m, 1H), 3.07-2.99 (m, 1H), 2.98-2.86 (m, 2H), 2.84 (s, 3H), 2.74-2.67 (m, 3H), 1.54-1.42 (m, 2H), 1.27-1.07 (m, 3H). LC-MS (ESI, m/z): [M+H]⁺ 439.

### Example-3-1-03: 2-({[(9H-fluoren-9-yl)methoxy]carbonyl}(4-methanesulfonamidobutyl)amino)acetic acid

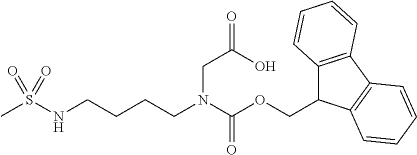 [SMILES: CS(=O)(=O)NCCCCN(CC(=O)O)C(=O)OCC1c2ccccc2-c2ccccc21]

Example-3-1-03 was synthesized as outlined for the preparation of Example-3-1-02 employing appropriate starting materials tert-butyl N-(4-aminobutyl)carbamate (CAS: 68076-36-8). ¹H NMR (400 MHz, DMSO-d₆) δ 12.67 (br, 1H), 7.91-7.87 (m, 2H), 7.67-7.60 (m, 2H), 7.42 (t, J=7.3 Hz, 2H), 7.38-7.29 (m, 2H), 6.98-6.90 (m, 1H), 4.45 (d, J=5.6 Hz, 1H), 4.26 (m, 2H), 3.89 (m, 2H), 3.25 (t, J=6.9 Hz, 1H), 2.99-2.90 (m, 2H), 2.88 (s, 3H), 2.84-2.76 (m, 1H), 1.57-1.37 (m, 2H), 1.26-1.11 (m, 2H). LC-MS (ESI, m/z): [M+H]⁺ 447.

### Example-3-1-04: 2-({[(9H-fluoren-9-yl)methoxy]carbonyl}(3-methanesulfonamidopropyl)amino)acetic acid

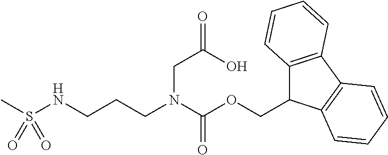 [SMILES: CS(=O)(=O)NCCCN(CC(=O)O)C(=O)OCC1c2ccccc2-c2ccccc21]

To a solution of N-(3-aminopropyl)methanesulfonamide (CAS: 88334-76-3, 0.50 g, 3.28 mmol) in AcOH (0.32 mL) and MeOH (16.1 mL) at rt was added 2-oxoacetic acid hydrate (CAS: 1152495-30-1, 0.30 mg, 3.28 mmol) and sodium cyanoborohydride (CAS: 25895-60-7, 0.14 mg, 2.20 mmol). The mixture was stirred at rt for 6 h, and then concentrated. To a solution of the resulting residue (0.69 g, 3.28 mmol) in 1,4-dioxane (16.4 mL) and H₂O (16.4 mL) at 0° C. was added NaHCO₃ (1.10 g, 13.1 mmol), followed by addition of Fmoc-OSu (1.00 g, 2.95 mmol). The mixture was stirred at rt for 2 h. The reaction mixture was acidified with 2M aq. HCl at 0° C. The mixture was extracted twice with EtOAc. The organic extracts were washed with brine, dried over Na₂SO₄, filtered, and concentrated. The resulting residue was purified by silica gel flash column chromatography (gradient, petroleum ether/EtOAc=99/1 to 50/50) to afford the title compound (0.18 g, 0.35 mmol). ¹H NMR (500 MHz, DMSO-d₆) δ 12.84 (br, 1H), 7.91-7.88 (m, 2H), 7.67-7.61 (m, 2H), 7.43-7.30 (m, 4H), 6.98-6.91 (m, 1H), 4.40-4.19 (m, 3H), 3.96 (s, 1H), 3.84 (s, 1H), 3.28-2.76 (m, 7H), 1.47-1.42 (m, 1H), 1.24-1.16 (m, 1H). LC-MS (ESI, m/z): [M+H]⁺ 433.

### Example-3-1-05: 2-({[(9H-fluoren-9-yl)methoxy]carbonyl}[3-(N-methylmethanesulfonamido)propyl]amino)acetic acid

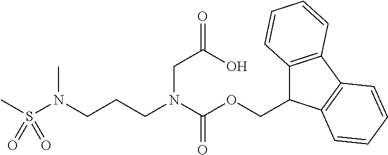 [SMILES: CN(CCCN(CC(=O)O)C(=O)OCC1c2ccccc2-c2ccccc21)S(C)(=O)=O]

To a solution of N-(3-aminopropyl)-N-methylmethanesulfonamide hydrochloride (CAS: 1955554-00-3, 0.51 g, 2.52 mmol) and sodium methoxide (0.14 g, 2.52 mmol) in AcOH (0.25 mL) in MeOH (12.4 mL) at rt was added 2-oxoacetic acid hydrate (CAS: 1152495-30-1, 0.23 g, 2.52 mmol) and sodium cyanoborohydride (0.11 g, 1.69 mmol). The mixture was stirred at rt for 6 h, and then concentrated. To a solution of the resulting residue (0.57 g, 2.52 mmol) in 1,4-dioxane (12.6 mL) and H₂O (12.6 mL) at 0° C. was added NaHCO₃ (0.85 g, 10.1 mmol), followed by a solution of Fmoc-OSu (0.77 g, 2.27 mmol). The mixture was stirred at rt for 2 h. The reaction mixture was acidified with 2M aq. HCl at 0° C. The mixture was extracted twice with EtOAc. The organic extracts were washed with brine, dried over Na₂SO₄, filtered, and concentrated. The resulting residue was purified by silica gel flash column chromatography (gradient, petroleum ether/EtOAc=99/1 to 50/50) to afford the title compound (0.43 g, 0.88 mmol). ¹H NMR (500 MHz, DMSO-d₆) δ 12.77 (br, 1H), 7.95-7.88 (m, 2H), 7.67-7.62 (m, 2H), 7.43-7.30 (m, 4H), 4.43-4.42 (m, 1H), 4.32-4.21 (m, 2H), 3.96 (s, 1H), 3.84 (s, 1H), 3.07-2.59 (m, 10H), 1.77-1.71 (m, 1H), 1.52-1.50 (m, 1H). LC-MS (ESI, m/z): [M+H]⁺ 448.

### Example-3-1-06: 2-[(4-acetamidobutyl)({[(9H-fluoren-9-yl)methoxy]carbonyl})amino]acetic acid

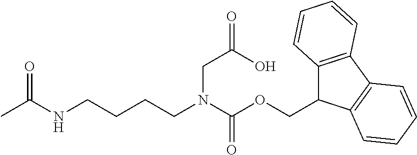 [SMILES: CC(=O)NCCCCN(CC(=O)O)C(=O)OCC1c2ccccc2-c2ccccc21]

To a solution of tert-butyl N-(4-aminobutyl)carbamate (CAS: 68076-36-8, 10.0 g, 53.1 mmol) in CH₃CN (150 mL) at 0° C. was added tert-butyl 2-bromoacetate (CAS: 5292-43-3, 15.5 g, 79.7 mmol) and DIPEA (18.5 mL, 106.2 mmol). The mixture was stirred at rt overnight. The mixture was extracted with EtOAc. The combined organic extracts were washed with brine, dried over Na₂SO₄, filtrated, and concentrated. The resulting residue was purified by silica gel flash column chromatography (isocratic, petroleum ether/EtOAc=50/50). Appropriate fractions were concentrated. To a solution of the obtained material (7.00 g, 23.1 mmol) in 1,4-dioxane (150 mL) at 0° C. was added H₂O (150 mL), and NaHCO₃ (3.89 g, 46.3 mmol), followed by Fmoc-Cl (CAS: 28920-43-6, 7.19 g, 27.8 mmol). The mixture was stirred at rt overnight. The pH of the mixture was adjusted to 6 with aq. citric acid. The mixture was extracted twice with EtOAc. The organic extracts were washed with brine, dried over Na₂SO₄, filtered, and concentrated. The resulting residue was purified by silica gel flash column chromatography (isocratic, petroleum ether/EtOAc=70/30). Appropriate fractions were concentrated. The mixture of the resulting residue (9.00 g, 17.1 mmol) and 12M aq. HCl (53 mL) in 1,4-dioxane (117 mL) was stirred at rt for 2 h, and then concentrated. To a solution of the resulting residue (5.00 g, 13.6 mmol) in THF (150 mL) at 0° C. was added BSA (11.0 g, 54.3 mmol). The mixture was stirred at 0° C. for 2 h. To the mixture at 0° C. was added DIPEA (7.09 mL, 40.7 mmol) and acetyl chloride (1.94 mL, 27.1 mmol). The mixture was stirred at rt for 2 h. The mixture was diluted with EtOAc (200 mL) and the pH of the mixture was adjusted to 5 with aq. citric acid. The mixture was washed three times with brine. The resulting solution was extracted with EtOAc. The organic extracts were dried over Na₂SO₄, filtered, and concentrated. The resulting residue was purified by reversed phase silica gel flash column chromatography (C18 silica gel, gradient, H₂O/CH₃CN=95/5 to 0/100) to afford the title compound (3.08 g, 7.50 mmol). ¹H NMR (400 MHz, DMSO-d₆) δ 12.63 (br, 1H), 7.93-7.86 (m, 2H), 7.83-7.70 (m, 1H), 7.68-7.59 (m, 2H), 7.45-7.29 (m, 4H), 4.44 (d, J=5.6 Hz, 1H), 4.33-4.17 (m, 2H), 3.98-3.78 (m, 2H), 3.24 (t, J=7.1 Hz, 1H), 3.05-2.87 (m, 3H), 1.78 (s, 3H), 1.53-1.28 (m, 2H), 1.21-1.03 (m, 2H). LC-MS (ESI, m/z): [M+H]⁺ 411.

### Intermediate-3-1-01: tert-butyl 2-[(3-{[(tert-butoxy)carbonyl](methyl)amino}propyl)({[(9H-fluoren-9-yl)methoxy]carbonyl})amino]acetate

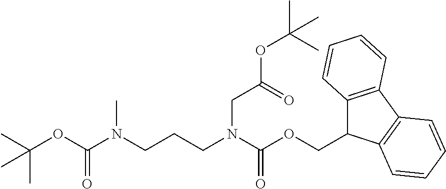 [SMILES: CN(CCCN(CC(=O)OC(C)(C)C)C(=O)OCC1c2ccccc2-c2ccccc21)C(=O)OC(C)(C)C]

To a solution of tert-butyl N-(3-aminopropyl)-N-methylcarbamate (CAS: 150349-36-3, 1.00 g, 5.31 mmol) in CH₃CN (26.6 mL) at 0° C. was added DIPEA (1.86 mL, 10.6 mmol) and tert-butyl 2-bromoacetate (CAS: 5292-43-3, 0.78 mL, 5.31 mmol). The mixture was stirred at rt for 10 h. The reaction was quenched satd. aq NaHCO₃. The mixture was extracted twice with EtOAc. The combined organic extracts were washed with brine, dried over Na₂SO₄, filtrated, and concentrated. To a solution of the resulting residue (1.61 g, 5.31 mmol) in H₂O (26.6 mL) at 0° C. was added NaHCO₃ (1.34 g, 15.9 mmol), followed by a solution of Fmoc-OSu (1.61 g, 4.78 mmol) in 1,4-dioxane (26.6 mL) dropwise. The mixture was stirred at rt for 20 h. The reaction mixture was acidified with 1M aq. HCl at 0° C. The mixture was extracted with EtOAc. The organic extracts were washed with brine, dried over Na₂SO₄, filtered, and concentrated. The resulting residue was purified by silica gel flash column chromatography (gradient, hexane/EtOAc=80/20 to 67/33) to afford the title compound (2.56 g, 4.88 mmol). LC-MS (ESI, m/z): [M+H]⁺ 525.

### Example-3-1-07: 2-({[(9H-fluoren-9-yl)methoxy]carbonyl}[3-(N-methylacetamido)propyl]amino)acetic acid

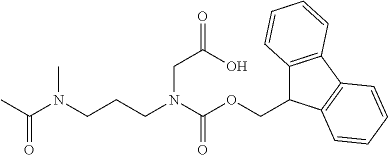 [SMILES: CC(=O)N(C)CCCN(CC(=O)O)C(=O)OCC1c2ccccc2-c2ccccc21]

The mixture of Intermediate-3-1-01 (1.08 g, 2.06 mmol) and 4M HCl in 1,4-dioxane (10.3 mL) was stirred at rt for 2 h, and then concentrated. To a solution of the resulting residue (0.88 g, 2.06 mmol) in THF (10.3 mL) and H₂O (10.3 mL) at 0° C. was added NaHCO₃ (0.69 g, 8.23 mmol), followed by acetic anhydride (0.42 g, 4.12 mmol). The mixture was stirred at 0° C. for 2 h. The pH of the mixture was adjusted to 1 with 1M aq. HCl. The mixture was extracted with EtOAc. The organic extracts were washed with brine, dried over Na₂SO₄, filtered, and concentrated. The resulting residue was purified by silica gel flash column chromatography (isocratic, CH₂Cl₂/MeOH=91/9) to afford the title compound (0.66 g, 1.62 mmol). ¹H NMR (500 MHz, DMSO-d₆) δ 12.62 (br, 1H), 7.90-7.88 (m, 2H), 7.66-7.61 (m, 2H), 7.43-7.30 (m, 4H), 4.56-4.55 (m, 1H), 4.29-4.21 (m, 2H), 3.98-3.80 (m, 2H), 3.28-2.63 (m, 7H), 1.96-1.84 (m, 3H), 1.73-1.36 (s, 2H). LC-MS (ESI, m/z): [M+H]⁺ 411.

### Example-3-1-08: 2-[(3-{[(tert-butoxy)carbonyl](methyl)amino}propyl)({[(9H-fluoren-9-yl)methoxy]carbonyl})amino]acetic acid

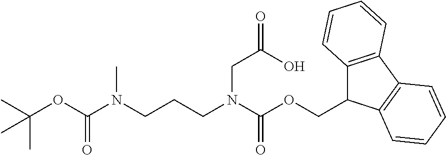 [SMILES: CN(CCCN(CC(=O)O)C(=O)OCC1c2ccccc2-c2ccccc21)C(=O)OC(C)(C)C]

The mixture of Intermediate-3-1-01 (0.79 g, 1.51 mmol) and 4M HCl in 1,4-dioxane (7.5 mL) was stirred at rt for 2 h, and then concentrated. To a solution of the resulting residue (0.61 g, 1.51 mmol) in THF (7.53 mL) and H₂O (7.53 mL) at 0° C. was added NaHCO₃ (0.51 g, 6.02 mmol), followed by di-tert-butyl dicarbonate (0.39 g, 1.81 mmol). The mixture was stirred at 0° C. for 2 h. The pH of the mixture was adjusted to 1 with 1M aq. HCl. The mixture was extracted with EtOAc. The organic extracts were washed with brine, dried over Na₂SO₄, filtered, and concentrated. The resulting residue was purified by silica gel flash column chromatography (isocratic, CH₂Cl₂/MeOH=91/9) to afford the title compound (0.55 g, 1.18 mmol). ¹H NMR (500 MHz, DMSO-d₆) δ 12.78 (br, 1H), 7.90-7.88 (m, 2H), 7.66-7.61 (m, 2H), 7.43-7.29 (m, 4H), 4.45-4.20 (m, 3H), 3.94 (s, 1H), 3.82 (s, 1H), 3.22-2.66 (m, 7H), 1.70-1.32 (m, 11H). LC-MS (ESI, m/z): [M+H]⁺ 469.

### Example-3-2-01: (2S)-3-{1-[(tert-butoxy)carbonyl]-5-phenoxy-1H-indol-3-yl}-2-({[(9H-fluoren-9-yl)methoxy]carbonyl}amino)propanoic acid

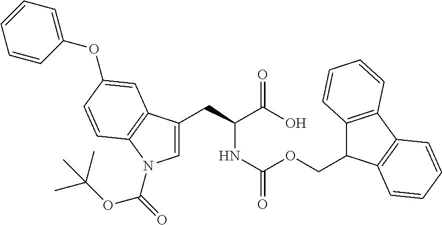 [SMILES: CC(C)(C)OC(=O)n1cc(C[C@H](NC(=O)OCC2c3ccccc3-c3ccccc32)C(=O)O)c2cc(Oc3ccccc3)ccc21]

To a suspension of 5-phenoxy-1H-indole (CAS: 78304-53-7, 7.3 g, 34.9 mmol) and KOH (4.9 g, 87.2 mmol) in DMF (100 mL) were added the solution of I₂ (8.85 g, 34.9 mmol) in DMF slowly. The mixture was stirred at rt for 45 min. The reaction was then quenched with water/ice. The mixture was extracted three times with EtOAc. The combined organic extracts were washed with brine, dried over Na₂SO₄, filtered, and concentrated. The resulting residue was purified by silica gel flash column chromatography (isocratic, petroleum ether/EtOAc=50/50). Appropriate fractions were concentrated. To a solution of the obtained material (11.6 g, 34.9 mmol) in CH₂Cl₂ (100 mL) were added Di-tert-butyl dicarbonate (CAS: 24424-99-5, 9.14 g, 41.9 mmol), DMAP (426 mg, 3.49 mmol), triethylamine (CAS: 121-44-8, 7.1 g, 69.8 mmol). The mixture was stirred at rt for 1 h. The mixture was quenched with water. The mixture was extracted three times with EtOAc. The combined organic extracts were washed with brine, dried over Na₂SO₄, filtered, and concentrated. The resulting residue was purified by silica gel flash column chromatography (isocratic, petroleum ether/EtOAc=50/50). Appropriate fractions were collected and concentrated. To a suspension of Zn (3.73 g, 57.4 mmol) in DMF (50 mL) at rt were added 1,2-dibromoethane (216 mg, 1.15 mmol) and chlorotrimethylsilane (250 mg, 2.30 mmol). The mixture was stirred at 60° C. for 30 min. To the mixture at rt was added methyl (R)-2-((((9H-fluoren-9-yl)methoxy)carbonyl)amino)-3-iodopropanoate (CAS: 156017-42-4, 6.22 g, 13.9 mmol). The mixture was stirred at rt for 1 h. To a mixture of obtained material in the last step (5.0 g, 11.5 mmol), Pd₂(dba)₃ CHCl₃ adduct (CAS: 52522-40-4, 595 mg, 0.57 mmol), and SPhos (471 mg, 1.15 mmol) in DMF (50 mL) at rt was added the organozinc reagent. The mixture was stirred at 30° C. overnight. The reaction was quenched with EtOAc and water at rt. The mixture was filtered. The filtrate was extracted with EtOAc. The organic extract was washed with brine, dried over Na₂SO₄, filtered, and then concentrated. The resulting residue was purified by silica gel flash column chromatography (gradient, CH₂Cl₂/MeOH=100/0 to 90/10). Appropriate fractions were concentrated. To a mixture of the obtained material (6.00 g, 9.48 mmol) in 2-propanol (60 mL) and H₂O (20 mL) at 0° C. were added CaCl₂ (10.5 g, 94.8 mmol) and LiOH·H₂O (1.82 g, 75.9 mmol). The mixture was stirred at rt for 3 h. The pH of the mixture was adjusted to pH<3 with 1M aq. HCl. The resulting mixture was extracted three times with EtOAc. The combined organic extracts were washed with aq. NaHCO₃ and brine, dried over Na₂SO₄, filtered, and then concentrated. The resulting residue was purified by silica gel flash column chromatography (isocratic, petroleum ether/EtOAc=50/50), and then further purified by reversed phase silica gel flash column chromatography (C18 silica gel, gradient, H₂O/CH₃CN=95/5 to 0/10 to afford the title compound (2.11 g, 3.41 mmol). ¹H NMR (400 MHz, DMSO-d₆) δ 11.9 (br, 1H), 8.05 (d, J=9.0 Hz, 1H), 7.95-7.79 (m, 3H), 7.71-7.56 (m, 3H), 7.44-7.21 (m, 7H), 7.14-7.01 (m, 2H), 7.01-6.91 (m, 2H), 4.36-4.10 (m, 4H), 3.22-2.93 (m, 2H), 1.58 (s, 9H). LC-MS (ESI, m/z): [M+H]⁺ 619.

### Example-3-2-02: (2S)-2-({[(9H-fluoren-9-yl)methoxy]carbonyl}amino)-3-[6-(pyridin-3-yl)-1H-indol-3-yl]propanoic acid

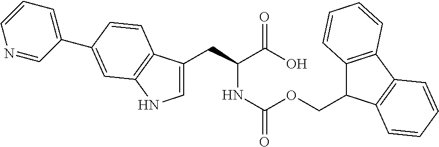 [SMILES: O=C(N[C@@H](Cc1c[nH]c2cc(-c3cccnc3)ccc12)C(=O)O)OCC1c2ccccc2-c2ccccc21]

To a solution of (2S)-3-(6-bromo-1H-indol-3-yl)-2-[(tert-butoxycarbonyl)amino]propanoic acid (CAS: 97444-12-7, 6.00 g, 15.7 mmol) in 1,4-dioxane (160 mL) at rt were added KOAc (4.61 g, 47.0 mmol), Pd(dppf)Cl₂ (1.15 g, 1.57 mmol), and stirred at rt for 5 min. To the mixture was added at rt bis(pinacolato)diboron (CAS: 73183-34-3, 7.95 g, 31.3 mmol). The resulting solution was stirred at 80° C. overnight. The reaction mixture was cooled to 25° C. and the pH of the mixture was adjusted to pH 6-7 with 20% aq. citric acid. The mixture was extracted with EtOAc. The organic extract was washed with brine, dried over Na₂SO₄, filtered, and then concentrated. The resulting residue was purified by silica gel flash column chromatography (gradient, hexane/EtOAc=100/0 to 50/50). Appropriate fractions were concentrated. To the resulting residue (1.60 g, 3.72 mmol) in DME (30 mL) and H₂O (10 mL) at rt were added Na₂CO₃ (1.18 g, 11.1 mmol) and Pd(dppf)Cl₂ (272 mg, 0.37 mmol), and stirred at rt for 5 min. To the mixture at rt was added 3-bromopyridine (CAS: 626-55-1, 882 mg, 5.58 mmol). The mixture was stirred at 90° C. for 3 h. The reaction mixture was cooled to 5-10° C. and the pH of the mixture was adjusted to pH 6-7 with 20% aq. citric acid. The mixture was extracted with EtOAc. The organic extract was washed with brine, dried over Na₂SO₄, filtered, and then concentrated. The resulting residue was purified by silica gel flash column chromatography (gradient, CH₂Cl₂/MeOH=100/0 to 94/6). Appropriate fractions were concentrated. To the resulting residue (1.08 g, 2.83 mmol) in CH₂Cl₂ (15 mL) at 0° C. was added TFA (15 mL). The resulting solution was stirred at rt for 1 h, and then concentrated. This resulting residue (3.94 g, 4.00 mmol) in 1,4-dioxane (60 mL) and H₂O (20 mL) at rt was added NaHCO₃ (672 mg, 8.00 mmol), and stirred at rt for 5 min. To the mixture at rt was added Fmoc-OSu (1.86 g, 5.51 mmol). The mixture was stirred at rt for 3 h. The pH of the mixture was adjusted to pH 6-7 with 20% aq. citric acid. The mixture was extracted with EtOAc. The organic extract was washed with brine, dried over Na₂SO₄, filtered, and then concentrated. The resulting residue was purified by reversed phase silica gel flash column chromatography (C18 silica gel, gradient, H₂O/CH₃CN=90/10 to 40/60) to afford the title compound (1.05 g, 2.09 mmol). ¹H NMR (400 MHz, DMSO-d₆) δ 12.69 (br, 1H), 11.06 (s, 1H), 8.90 (s, 1H), 8.54 (d, J=4.7 Hz, 1H), 8.15 (s, 1H), 7.89 (d, J=7.6 Hz, 2H), 7.78-7.14 (m, 12H), 4.31-4.07 (m, 4H), 3.29-3.02 (m, 2H). LC-MS (ESI, m/z): [M+H]⁺ 504.

The following compounds were synthesized as outlined for the preparation of Example-3-2-02 employing appropriate starting materials in the table.

| | Structure and IUPAC name |
|---|---|
| | Boc-protected starting material-A |
| | Starting material-B |
| Example | NMR and MS |
| 3-2-03 | 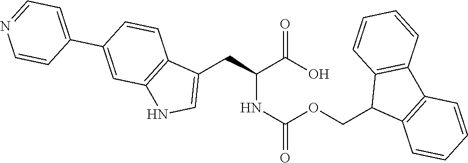 [SMILES: O=C(N[C@@H](Cc1c[nH]c2cc(-c3ccncc3)ccc12)C(=O)O)OCC1c2ccccc2-c2ccccc21] |
| | (2S)-2-({[(9H-fluoren-9-yl)methoxy]carbonyl}amino)-3-[6-(pyridin-4-yl)- |
| | 1H-indol-3-yl]propanoic acid |
| | (2S)-3-(6-bromo-1H-indol-3-yl)-2-[(tert-butoxycarbonyl)amino]propanoic |
| | acid (CAS: 97444-12-7) |
| | 4-bromopyridine (CAS: 1120-87-2) |
| | ¹H NMR (400 MHZ, DMSO-d₆) δ 12.75 (br, 1H), 11.12 (s, 1H), 8.60 (d, |
| | J = 6.0 Hz, 2H), 7.90 (d, J = 7.6 Hz, 2H), 7.82-7.63 (m, 6H), 7.52-7.11 |
| | (m, 7H), 4.31-4.05 (m, 4H), 3.29-2.97 (m, 2H). LC-MS (ESI, m/z): |
| | [M + H]⁺ 504. |
| 3-2-04 | 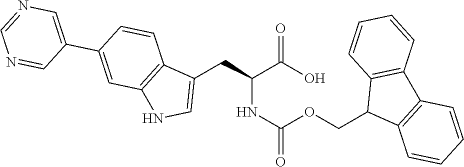 [SMILES: O=C(N[C@@H](Cc1c[nH]c2cc(-c3cncnc3)ccc12)C(=O)O)OCC1c2ccccc2-c2ccccc21] |
| | (2S)-2-({[(9H-fluoren-9-yl)methoxy]carbonyl}amino)-3-[6-(pyrimidin-5- |
| | yl)-1H-indol-3-yl]propanoic acid |
| | (2S)-3-(6-bromo-1H-indol-3-yl)-2-[(tert-butoxycarbonyl)amino]propanoic |
| | acid (CAS: 97444-12-7) |
| | 5-bromopyrimidine (CAS: 4595-59-9) |
| | ¹H NMR (400 MHz, DMSO-d₆) δ 12.82 (br, 1H), 11.15 (s, 1H), 9.16 (br, |
| | 3H), 7.99-7.63 (m, 7H), 7.60-7.24 (m, 6H), 4.43-4.02 (m, 4H), 3.55- |
| | 2.97 (m, 2H). LC-MS (ESI, m/z): [M + H]⁺ 505. |

### Example-3-2-05: (2S)-3-{1-[(tert-butoxy)carbonyl]-6-(1-methyl-1H-pyrazol-4-yl)-1H-indol-3-yl}-2-({[(9H-fluoren-9-yl)methoxy]carbonyl}amino)propanoic acid

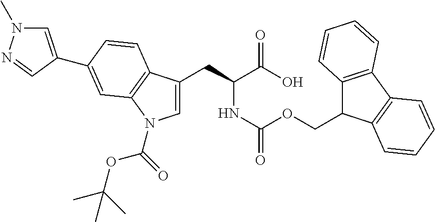 [SMILES: Cn1cc(-c2ccc3c(C[C@H](NC(=O)OCC4c5ccccc5-c5ccccc54)C(=O)O)cn(C(=O)OC(C)(C)C)c3c2)cn1]

To a mixture of 6-(1-methyl-1H-pyrazol-4-yl)-1H-indole (CAS: 1847462-94-5, 4.06 g, 20.6 mmol), KOH (2.89 g, 51.5 mmol) in DMF (50 ml) at 0° C. were added a solution of I₂ (5.22 g, 20.6 mmol) in DMF (50 ml) and stirred at 0° C. for 10 min and at rt for 30 min. The mixture was quenched with satd. aq. Na₂S₂O₃. The mixture was extracted twice with EtOAc. The combined organic extracts were washed with water and brine, dried over Na₂SO₄, filtered, and concentrated. To a solution of the resulting residue (6.66 g, 20.6 mmol) in CH₂Cl₂ (41.2 ml) at 0° C. were added di-tert-butyl dicarbonate (CAS: 24424-99-5, 5.74 ml, 24.7 mmol), triethylamine (5.74 ml, 41.2 mmol), and DMAP (0.252 g, 2.06 mmol). The mixture was stirred at 0° C. for 5 min and at rt for 1 h. The mixture was concentrated. The resulting residue was purified by silica gel flash column chromatography (gradient, hexane/EtOAc=100/0 to 40/60). Appropriate fractions were collected and concentrated. To a suspension of Zn (2.62 g, 40.0 mmol) in DMF (10 mL) at rt were added chlorotrimethylsilane (174 mg, 1.60 mmol). The mixture was stirred at rt for 30 min. To the mixture at rt was added methyl (R)-2-((((9H-fluoren-9-yl)methoxy)carbonyl)amino)-3-iodopropanoate (CAS: 156017-42-4, 4.34 g, 9.61 mmol). The mixture was stirred at rt for 1 h. To a mixture of obtained material in the last step (3.39 g, 8.01 mmol), Pd₂(dba)₃ CHCl₃ adduct (332 mg, 0.32 mmol), and SPhos (263 mg, 0.64 mmol) in DMF (10 mL) at rt was added the organozinc reagent. The mixture was stirred at 50° C. for 30 min and at 80° C. for 30 min. The reaction was quenched at rt with EtOAc and water. The mixture was filtered. The filtrate was extracted with EtOAc. The organic extract was washed with brine, dried over Na₂SO₄, filtered, and then concentrated. The resulting residue was purified by silica gel flash column chromatography (gradient, hexane/EtOAc=90/10 to 0/100). Appropriate fractions were concentrated. To the resulting residue (3.51 g, 5.66 mmol) in ClCH₂CH₂Cl (28.3 mL) at rt was added trimethyltin hydroxide (CAS: 56-24-6, 2.56 g, 14.2 mmol). The mixture was stirred at 40° C. for 18 h, and then concentrated. The mixture was poured into 1M aq. HCl. The mixture was extracted twice with CH₂Cl₂. The combined organic extracts were washed with brine, dried over Na₂SO₄, filtered, and then concentrated. The resulting residue was purified by silica gel flash column chromatography (gradient, hexane/EtOAc=60/40 to 0/100) to afford the title compound (2.46 g, 3.86 mmol). ¹H NMR (500 MHz, DMSO-d₆) δ 12.8 (br, 1H), 8.18 (s, 1H), 8.12 (s, 1H), 7.87 (d, J=7.6 Hz, 2H), 7.84 (d, J=8.3 Hz, 1H), 7.80 (s, 1H), 7.69-7.57 (m, 3H), 7.52 (s, 1H), 7.46 (d, J=8.0 Hz, 1H), 7.43-7.33 (m, 2H), 7.27 (t, J=7.4 Hz, 1H), 7.23 (t, J=7.5 Hz, 1H), 4.37-4.27 (m, 1H), 4.25-4.14 (m, 3H), 3.88 (s, 3H), 3.17 (m, 1H), 3.02 (m, 1H), 1.59 (s, 9H) LC-MS (ESI, m/z): [M+H]⁺ 607.

### Example-3-2-06: (2S)-3-(3-carbamoyl-4-hydroxyphenyl)-2-({[(9H-fluoren-9-yl)methoxy]carbonyl}amino)propanoic acid

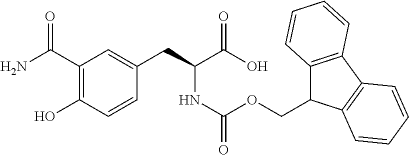 [SMILES: NC(=O)c1cc(C[C@H](NC(=O)OCC2c3ccccc3-c3ccccc32)C(=O)O)ccc1O]

To a suspension of Zn (54.5 g, 833 mmol) in DMF (2000 mL) at rt was added I₂ (10.6 g, 41.7 mmol). The mixture was stirred at rt for 15 min. To the mixture at rt was added methyl (R)-2-((((9H-fluoren-9-yl)methoxy)carbonyl)amino)-3-iodopropanoate (CAS: 156017-42-4, 125.3 g, 278 mmol) and I₂ (10.6 g, 41.7 mmol). The mixture was stirred at rt for 1 h. To the mixture at rt were added 5-bromo-2-hydroxybenzamide (CAS: 6329-74-4, 60.0 g, 278 mmol), Pd₂(dba)₃ CHCl₃ adduct (12.7 g, 13.9 mmol), and SPhos (11.4 g, 27.8 mmol). The mixture was stirred at 50° C. for 4 h. The mixture was quenched with H₂O. The mixture was extracted three times with EtOAc. The organic extracts were washed with brine, dried over Na₂SO₄, filtered, and then concentrated. The resulting residue was purified by silica gel flash column chromatography (isocratic, petroleum ether/EtOAc=25/75). Appropriate fractions were concentrated. To a mixture of the obtained material (90.0 g, 195 mmol) in 2-propanol (900 mL), THF (300 mL), and H₂O (200 mL) at 5° C. were added CaCl₂) (346 g, 3.12 mol) and LiOH·H₂O (33.0 g, 786 mmol). The mixture was stirred at rt overnight. The pH of the mixture was adjusted to pH 4 with sartd. aq. NaH₂PO₄. The resulting mixture was extracted three times with EtOAc. The combined organic extracts were washed three times with H₂O, dried over Na₂SO₄, filtered, and then concentrated. The resulting residue was purified by silica gel flash column chromatography (isocratic, CH₂Cl₂/MeOH=95/5), to afford the title compound (53 g, 119 mmol). ¹H NMR (400 MHz, DMSO-d₆) δ 12.83 (br, 1H), 8.38 (br, 1H), 8.08-7.74 (m, 4H), 7.70-7.61 (m, 3H), 7.57-7.10 (m, 6H), 6.79 (d, J=8.4 Hz, 1H), 4.39-4.08 (m, 4H), 3.17-2.64 (m, 2H). LC-MS (ESI, m/z): [M+H]⁺ 447.

### Example-3-2-07: (2S)-3-[3-(dimethylcarbamoyl)-4-hydroxyphenyl]-2-({[(9H-fluoren-9-yl)methoxy]carbonyl}amino)propanoic acid

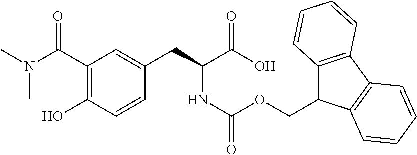 [SMILES: CN(C)C(=O)c1cc(C[C@H](NC(=O)OCC2c3ccccc3-c3ccccc32)C(=O)O)ccc1O]

To a suspension of Zn (22.5 g, 344 mmol) in DMF (280 mL) was added I₂ (4.37 g, 17.2 mmol). To the mixture at rt was added methyl (R)-2-((((9H-fluoren-9-yl)methoxy)carbonyl)amino)-3-iodopropanoate (CAS: 156017-42-4, 77.7 g, 172 mmol) and I₂ (4.37 g, 17.2 mmol). The mixture was stirred at rt for 30 min. To the mixture were added 5-bromo-2-hydroxy-N,N-dimethylbenzamide (CAS: 473731-66-7, 28 g, 114 mmol), Pd₂(dba)₃ (2.63 g, 2.87 mmol), and SPhos (2.35 g, 5.74 mmol) in DMF (280 mL). The mixture was stirred at 60° C. for 2.5 h. The mixture was quenched with H₂O. The mixture was extracted three times with EtOAc. The organic extracts were washed with brine, dried over Na₂SO₄, filtered, and then concentrated. The resulting residue was purified by silica gel flash column chromatography (gradient, petroleum ether/EtOAc=40/60 to 20/80). Appropriate fractions were concentrated. To a solution of the resulting residue (40 g, 81.9 mmol) in 1,4-dioxane (800 mL) was added conc. aq. HCl (400 mL) and stirred at 100° C. for 1 h. The reaction was then diluted with water. The mixture was extracted three times with EtOAc. The combined organic extracts were washed with brine, dried over Na₂SO₄, filtered, and then concentrated. The resulting residue was purified by reversed phase silica gel flash column chromatography (C18 silica gel, gradient, CH₃CN/H₂O with NH₄HCO₃=45/55 to 65/35) to afford the title compound (19.7 g, 41.5 mmol). ¹H NMR (300 MHz, DMSO-d₆) δ 12.72 (br, 1H), 9.63 (s, 1H), 7.89 (d, J=7.5 Hz, 2H), 7.76-7.60 (m, 3H), 7.55-7.24 (m, 4H), 7.11 (dd, J=8.3, 2.3 Hz, 1H), 7.01 (d, J=2.2 Hz, 1H), 6.77 (d, J=8.3 Hz, 1H), 4.29-4.05 (m, 4H), 3.12-2.64 (m, 8H). LC-MS (ESI, m/z): [M+H]⁺ 475.

### Example-3-2-08: (2S)-2-({[(9H-fluoren-9-yl)methoxy]carbonyl}amino)-3-[4-(2-{[(prop-2-en-1-yloxy)carbonyl]amino}ethoxy)phenyl]propanoic acid

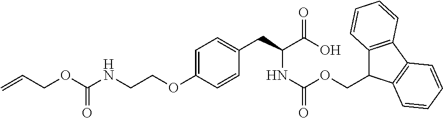 [SMILES: C=CCOC(=O)NCCOc1ccc(C[C@H](NC(=O)OCC2c3ccccc3-c3ccccc32)C(=O)O)cc1]

To a mixture of tert-butyl (2S)-2-[(tert-butoxycarbonyl)amino]-3-(4-hydroxyphenyl)propanoate (CAS: 18938-60-8, 47.1 g, 140 mmol) and potassium carbonate (48.2 g, 346 mmol) in CH₃CN (800 mL) at 0° C. was added prop-2-en-1-yl N-(2-bromoethyl)carbamate (CAS: 347372-90-1, 37.5 g, 180 mmol) dropwise. The resulting solution was stirred for 3 days at 50° C. The resulting mixture was concentrated. The resulting residue was purified by silica gel flash column chromatography (gradient, petroleum ether/EtOAc=99/1 to 75/25). Appropriate fractions were concentrated. To the resulting residue (64 g, 170 mmol) in CH₂Cl₂ (720 mL) was added TFA (360 mL). The resulting solution was stirred at rt for 12 h. The resulting mixture was concentrated. The pH of the mixture of resulting residue (80.3 g, 198 mmol) in 1,4-dioxane (650 mL) and H₂O (650 mL) was adjusted to pH 9 satd. aq. Na₂CO₃. To the mixture at 0° C. were added Na₂CO₃ (41.9 g, 395 mmol) and Fmoc-OSu (63.3 g, 188 mmol). The mixture was stirred at rt for 3 h. The reaction was quenched with water. The resulting mixture was extracted with EtOAc. The organic extract was washed three times with water, dried over Na₂SO₄, filtered, and then concentrated. The resulting residue was purified by reversed phase silica gel flash column chromatography (C18 silica gel, gradient, H₂O/CH₃CN with 1% formic acid=55/45 to 25/75) to afford the title compound (25.4 g, 47.9 mmol). ¹H NMR (400 MHz, DMSO-d₆) δ 12.7 (br, 1H), 7.88 (d, J=7.5 Hz, 2H), 7.75-7.60 (m, 3H), 7.58-7.38 (m, 3H), 7.38-7.25 (m, 2H), 7.18 (d, J=8.3 Hz, 2H), 6.84 (d, J=8.4 Hz, 2H), 6.02-5.79 (m, 1H), 5.31-5.24 (m, 1H), 5.19-5.12 (m, 1H), 4.48 (d, J=5.3 Hz, 2H), 4.31-4.07 (m, 4H), 3.93 (t, J=5.8 Hz, 2H), 3.42-3.26 (m, 2H), 3.10-2.70 (m, 2H). LC-MS (ESI, m/z): [M+H]⁺ 531.

### Example-3-3-01: (2S)-3-[4-(2-acetamidoethoxy)phenyl]-2-({[(9H-fluoren-9-yl)methoxy]carbonyl}amino)propanoic acid

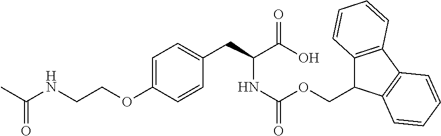 [SMILES: CC(=O)NCCOc1ccc(C[C@H](NC(=O)OCC2c3ccccc3-c3ccccc32)C(=O)O)cc1]

To a solution of (2S)-3-[4-(2-{[(tert-butoxy)carbonyl]amino}ethoxy)phenyl]-2-({[(9H-fluoren-9-yl)methoxy]carbonyl}amino)propanoic acid (CAS: 1013883-02-7, 50.0 g, 91.0 mmol) in DCM (100 mL) at rt was added 4M HCl in CPME (100 mL) and stirred for 3 h. The resulting solid was collected by filtration. The solid was washed by DCM and dried at 50° C. under vacuum. To a solution of the residue (17.6 g, 36.5 mmol) in 1,4-dioxane (122 mL) at 0° C. was added a solution of sodium carbonate (15.5 g, 146 mmol) in water (61.0 mL). To the suspension was transferred acetic anhydride (3.62 mL, 38.3 mmol) via a syringe and the resulting reaction mixture was stirred at 25° C. for 1 h after stirring for 10 min at 0° C. A pH value of the mixture was adjusted to 3 at 0° C. by an addition of 1M aq. HCl. The remaining solid was collected by filtration and the solid was washed with water and IPE. The solid was dried overnight at 50° C. under high vacuum to afford the title compound (16.0 g, 32.8 mmol) as a white solid. ¹H NMR (500 MHz, DMSO-d₆) δ 12.76 (br, 1H), 8.14-8.06 (m, 1H), 7.92-7.85 (m, 2H), 7.74-7.61 (m, 3H), 7.45-7.37 (m, 2H), 7.36-7.25 (m, 2H), 7.22-7.12 (m, 2H), 6.89-6.80 (m, 2H), 4.26-4.06 (m, 4H), 3.97-3.87 (m, 2H), 3.44-3.30 (m, 2H), 3.06-2.94 (m, 1H), 2.85-2.72 (m, 1H), 1.82 (s, 3H). LC-MS (ESI, m/z): [M+H]⁺ 489.

### Example-3-3-02: (2S)-3-(3-carbamoylpyridin-2-yl)-2-({[(9H-fluoren-9-yl)methoxy]carbonyl}amino)propanoic acid

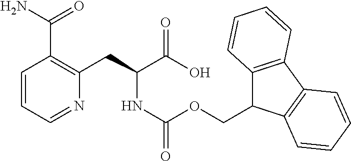 [SMILES: NC(=O)c1cccnc1C[C@H](NC(=O)OCC1c2ccccc2-c2ccccc21)C(=O)O]

To a suspension of Zn (25.5 g, 389 mmol) in DMF (250 mL) at rt were added 1,2-dibromoethane (2.06 mL, 24.1 mmol) and chlorotrimethylsilane (0.84 mL, 6.62 mmol). The mixture was stirred at 60° C. for 30 min. To the mixture at rt was added methyl (2R)-2-[[(tert-butoxy)carbonyl]amino]-3-iodopropanoate (CAS: 93267-04-0, 36.0 g, 109 mmol) in DMF (50 mL) dropwise with stirring in 30 min. To the mixture were added a solution of 2-bromopyridine-3-carbonitrile (CAS: 20577-26-8, 20.0 g, 109 mmol) in DMF (300 mL) and [Pd(allyl)Cl]₂ (3.0 g, 8.20 mmol). The resulting solution was stirred overnight at 50° C. The reaction was quenched with H₂O then diluted with EtOAc. The mixture was filtered. The filtrate was extracted with EtOAc. The organic extract was washed with brine, dried over Na₂SO₄, filtered, and then concentrated. The resulting residue was purified by silica gel flash column chromatography (gradient, CH₂Cl₂/MeOH=100/0 to 80/20), and then silica gel flash column chromatography (gradient, petroleum ether:EtOAc=90/10 to 80/20). Appropriate fractions were concentrated. To the resulting residue (15.0 g, 49.1 mmol) in DMSO (50 mL) at 0° C. were added K₂CO₃ (1.36 g, 9.81 mmol) and peroxol (10 mL) dropwise with stirring. The resulting solution was stirred for 1 h at rt. The reaction was then quenched by the addition of H₂O. The resulting solution was extracted with DCM:MeOH (10:1) and the combined organic layers were dried over MgSO₄ and concentrated under vacuum. The resulting residue was purified by silica gel flash column chromatography (gradient, petroleum ether:EtOAc=88:12 to 0:100). Appropriate fractions were concentrated. To a solution of the residue (14.0 g, 43.3 mmol) in H₂O (35 mL) at 0° C. was added aq. hydrogen chloride (18 mL) dropwise with stirring. The resulting solution was stirred for 4 h at rt. The pH value of the solution was adjusted to 7 with 1M aq. sodium hydroxide. The resulting mixture was concentrated under vacuum. To a solution of the resulting residue (8.00 g, 35.8 mmol) in 1,4-dioxane (100 mL) at 0° C. were added a solution of NaHCO₃ (6.03 g, 71.7 mmol) in H₂O (100 mL) and Fmoc-OSu (9.27 g, 35.8 mmol). The mixture was stirred for 2 h at rt, and then pH of the solution was adjusted to 4 with 2M aq. HCl at 0° C. The resulting solution was extracted with EtOAc and the combined organic layers washed with brine, dried over Na₂SO₄ and concentrated under vacuum. The resulting residue was purified by silica gel flash column chromatography (gradient, petroleum ether:EtOAc=80:20 to 0:100). Appropriate fractions were concentrated. To a mixture of the residue (10.0 g, 22.5 mmol) in THF (100 mL) 0° C. was added a solution of LiOH·H₂O (1.42 g, 33.9 mmol) in H₂O (100 mL) dropwise with stirring. The mixture was stirred for 2 h at rt. The pH of the mixture was adjusted to pH 4 with 2M aq. HCl. The mixture was concentrated under vacuum. The resulting residue was purified by reversed phase silica gel flash column chromatography (C18 silica gel, gradient, H₂O/CH₃CN=90/10 to 60/40) to afford the title compound (5.09 g, 11.8 mmol). ¹H NMR (300 MHz, Methanol-d₄) δ 8.65-8.49 (m, 1H), 7.90-7.74 (m, 3H), 7.72-7.54 (m, 2H), 7.45-7.21 (m, 5H), 4.67-4.52 (m, 1H), 4.27-4.07 (m, 3H), 3.57-3.29 (m, 2H). LC-MS (ESI, m/z): [M+H]⁺ 432.

### Example-3-3-03: (2S)-2-({[(9H-fluoren-9-yl)methoxy]carbonyl}amino)-3-(6-phenylpyridin-3-yl)propanoic acid

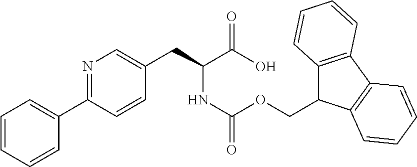 [SMILES: O=C(N[C@@H](Cc1ccc(-c2ccccc2)nc1)C(=O)O)OCC1c2ccccc2-c2ccccc21]

To a suspension of Zn (168 g, 2.56 mol) in DMF (2000 mL) was added I₂ (32.5 g, 128 mmol). The mixture was stirred at rt for 2 min. To the mixture at rt was added methyl (R)-2-((((9H-fluoren-9-yl)methoxy)carbonyl)amino)-3-iodopropanoate (CAS: 156017-42-4, 463 g, 1.03 mol). The mixture was stirred at rt for 1 h. To the mixture were added 5-bromo-2-phenylpyridine (CAS: 27012-25-5, 200 g, 854 mmol), Pd₂(dba)₃ CHCl₃ adduct (23.5 g, 25.6 mmol), and SPhos (17.5 g, 42.7 mmol). The mixture was stirred at 60° C. for an additional 3 h, and then filtered. The remaining solid was washed with EtOAc and the filtrate was diluted with etOAc. The mixture was washed three times with brine, dried over Na₂SO₄, filtered, and then concentrated. The resulting residue was purified by silica gel flash column chromatography (isocratic, CH₂Cl₂/petroleum ether/EtOAc=13:74:13). Appropriate fractions were concentrated. To a mixture of the residue (120 g, 251 mmol) in 2-propanol (1000 mL) and THF (300 mL) at 0° C. were added CaCl₂ (69.0 g, 622 mmol), followed by the addition of LiOH·H₂O (42.1 g, 1003 mmol) in H₂O (300 mL) dropwise. The mixture was stirred at rt for 16 h. The pH of the mixture was adjusted to pH 4 with satd. aq. NaH₂PO₄ (aq.). The resulting mixture was filtrated. The filtrate was extracted three times with EtOAc. The combined organic extracts were washed three times with brine, dried over Na₂SO₄, filtered, and then concentrated. The resulting residue was purified by trituration with Et₂O (500 mL) to afford the title compound (91.3 g, 197 mmol). ¹H NMR (400 MHz, DMSO-d₆) δ 12.88 (br, 1H), 8.58 (s, 1H), 8.16-8.01 (m, 2H), 7.95-7.70 (m, 5H), 7.70-7.58 (m, 2H), 7.57-7.19 (m, 7H), 4.34-4.08 (m, 4H), 3.24-3.08 (m, 1H), 3.03-2.86 (m, 1H). LC-MS (ESI, m/z): [M+H]⁺ 465.

The following compounds were synthesized as outlined for the preparation of Example-3-3-03 employing appropriate starting materials in the table.

| | Structure and IUPAC name |
|---|---|
| | Fmoc-protected starting material-A |
| Ex- | Starting material-B |
| ample | NMR and MS |
| 3-3- 04 | 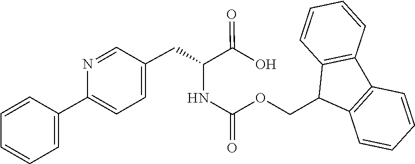 [SMILES: O=C(N[C@H](Cc1ccc(-c2ccccc2)nc1)C(=O)O)OCC1c2ccccc2-c2ccccc21] |
| | (2R)-2-({[(9H-fluoren-9-yl)methoxy]carbonyl}amino)-3- |
| | (6-phenylpyridin-3-yl)propanoic acid |
| | methyl (2S)-2-{[(9H-fluoren-9-ylmethoxy)carbonyl]amino}-3- |
| | iodopropanoate (CAS: 527696-77-1) |
| | 5-bromo-2-phenylpyridine |
| | ¹H NMR (400 MHZ, DMSO-d₆) δ 12.88 (br, 1H), 8.58 (s, 1H), |
| | 8.16-8.01 (m, 2H), 7.95-7.70 (m, 5H), 7.70-7.58 (m, 2H), 7.57-7.19 |
| | (m, 7H), 4.34-4.08 (m, 4H), 3.24-3.08 (m, 1H), 3.03-2.86 (m, 1H). |
| | LC-MS (ESI, m/z): [M + H]⁺ 465. |

### Example-3-3-05: (2S)-3-(2-carbamoylpyridin-4-yl)-2-({[(9H-fluoren-9-yl)methoxy]carbonyl}amino)propanoic acid

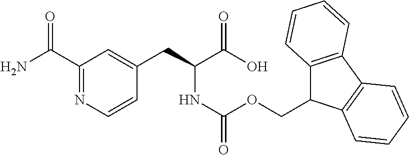 [SMILES: NC(=O)c1cc(C[C@H](NC(=O)OCC2c3ccccc3-c3ccccc32)C(=O)O)ccn1]

To a suspension of Zn (23.3 g, 356 mmol) in DMF (150 mL) at rt were added 1,2-dibromoethane (1.88 mL, 22.0 mmol) and chlorotrimethylsilane (0.765 mL, 6.03 mmol). The mixture was stirred at 60° C. for 30 min. To the mixture at rt was added methyl (2R)-2-[[(tert-butoxy)carbonyl]amino]-3-iodopropanoate (CAS: 93267-04-0, 32.9 g, 100 mmol) in DMF (50 mL) dropwise with stirring in 30 min. To the mixture were added a solution of 4-bromopyridine-2-carboxamide (CAS: 62150-46-3, 20.0 g, 99.0 mmol) in DMF (300 mL) and Pd(PPh₃)₂Cl₂ (3.50 g, 4.99 mmol). The resulting solution was stirred overnight at 50° C. The reaction was quenched with H₂O. The mixture was filtered. The filtrate was extracted with DCM:MeOH (10:1). The organic extract was washed with brine, dried over Na₂SO₄, filtered, and then concentrated. The resulting residue was purified by silica gel flash column chromatography (gradient, petroleum ether:EtOAc=83:17 to 0:100). Appropriate fractions were concentrated. To a mixture of the residue (15.0 g, 46.4 mmol) in MeOH (200 mL) at 0° C. was a solution of sodium hydroxide (1.86 g, 46.5 mmol) in H₂O (200 mL) dropwise. The mixture was stirred at 0° C. for 2 h. The pH of the mixture was adjusted to pH 4 with 2M aq. HCl. The resulting mixture was concentrated under vacuum. To a solution of the residue (16.0 g, 51.7 mmol) in H₂O (44 mL) at 0° C. was added aq. hydrogen chloride (22 mL) dropwise with stirring. The resulting solution was stirred for 4 h at rt. The pH value of the solution was adjusted to 7 with 1M aq. sodium hydroxide. The resulting mixture was concentrated under vacuum. To a solution of the residue (13.0 g, 62.1 mmol) in 1,4-dioxane (150 mL) at 0° C. were added a solution of NaHCO₃ (10.5 g, 124 mmol) in H₂O (150 mL) and Fmoc-Cl (16.1 g, 62.1 mmol). The mixture was stirred for 2 h at rt, and the resulting mixture was concentrated under vacuum. The resulting residue was purified by reversed phase silica gel flash column chromatography (C18 silica gel, gradient, H₂O/CH₃CN=80/20 to 40/60) to afford the title compound (5.00 g, 11.6 mmol). ¹H NMR (300 MHz, Methanol-da) δ 8.59-8.42 (m, 1H), 8.07 (s, 1H), 7.84-7.76 (m, 2H), 7.64-7.53 (m, 2H), 7.50-7.24 (m, 5H), 4.59-4.41 (m, 1H), 4.39-4.11 (m, 3H), 3.43-3.29 (m, 1H), 3.18-3.00 (m, 1H). LC-MS (ESI, m/z): [M+H]⁺ 432.

### Example-3-3-06: (2S)-3-(3-carbamoylpyridin-4-yl)-2-({[(9H-fluoren-9-yl)methoxy]carbonyl}amino)propanoic acid

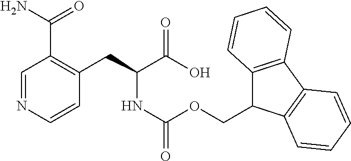 [SMILES: NC(=O)c1cnccc1C[C@H](NC(=O)OCC1c2ccccc2-c2ccccc21)C(=O)O]

To a suspension of Zn (36.9 g, 564 mmol) in DMF (300 mL) at rt were added 1,2-dibromoethane (3.09 mL, 36.2 mmol) and chlorotrimethylsilane (1.26 mL, 9.93 mmol). The mixture was stirred at 60° C. for 30 min. To the mixture at rt was added methyl (2R)-2-[[(tert-butoxy)carbonyl]amino]-3-iodopropanoate (CAS: 93267-04-0, 54.0 g, 164 mmol) in DMF (50 mL) dropwise with stirring in 30 min. To the mixture were added a solution of 4-bromopyridine-3-carbonitrile (CAS: 154237-70-4, 30.0 g, 164 mmol) in DMF (200 mL) and Pd(OAc)₂ (2.76 g, 12.3 mmol) and Xphos (11.7 g, 24.6 mmol). The resulting solution was stirred overnight at 50° C. The reaction was quenched with H₂O. The mixture was filtered. The filtrate was extracted with EtOAc. The organic extract was washed with brine, dried over Na₂SO₄, filtered, and then concentrated. The resulting residue was purified by silica gel flash column chromatography (gradient, petroleum ether:EtOAc=90:10 to 80:20). Appropriate fractions were concentrated. To the resulting residue (20.0 g, 65.5 mmol) in DMSO (100 mL) at 0° C. was added K₂CO₃ (1.81 g, 13.1 mmol) and aq. hydrogen peroxide (20 mL) dropwise with stirring. The resulting solution was stirred for 2 h at rt. The reaction was then quenched by the addition of H₂O. The resulting solution was extracted with DCM:MeOH (10:1) and the organic layers was dried over MgSO₄ and concentrated under vacuum. The resulting residue was purified by silica gel flash column chromatography (gradient, petroleum ether:EtOAc=80:20 to 0:100). Appropriate fractions were concentrated. To a solution of the residue (12.0 g, 38.8 mmol) in H₂O (31 mL) at 0° C. was added aq. hydrogen chloride (15.5 mL) dropwise with stirring. The resulting solution was stirred for 4 h at rt. The pH value of the solution was adjusted to 7 with 1M aq. sodium hydroxide. The resulting mixture was concentrated under vacuum. To a solution of the resulting residue (9.00 g, 43.0 mmol) in 1,4-dioxane (100 mL) and H₂O (100 mL) at 0° C. deg were added NaHCO₃ (6.78 g, 81.0 mmol) and Fmoc-Cl (10.4 g, 40.2 mmol). The mixture was stirred for 2 h at rt, and the resulting mixture was concentrated under vacuum. The resulting residue was purified by silica gel flash column chromatography (gradient, petroleum ether:EtOAc=83:17 to =0:100). Appropriate fractions were concentrated. To a mixture of the residue (11.0 g, 24.7 mmol) in THF (100 mL) at 0° C. was added a solution of LiOH·H2O (1.56 g, 37.1 mmol) in H₂O (100 mL). The mixture was stirred at rt for 2 h. The pH of the mixture was adjusted to pH 4 with 2M aq. HCl. The mixture was concentrated under vacuum. The resulting residue was purified by reversed phase silica gel flash column chromatography (C18 silica gel, gradient, H₂O/CH₃CN=90/10 to 60/40) to afford the title compound (5.09 g, 11.8 mmol). ¹H NMR (400 MHz, Methanol-d₄) δ 8.59 (s, 1H), 8.52-8.40 (m, 1H), 7.85-7.74 (m, 2H), 7.67-7.56 (m, 2H), 7.55-7.47 (m, 1H), 7.44-7.23 (m, 4H), 4.41-4.32 (m, 1H), 4.31-4.09 (m, 3H), 3.52-3.39 (m, 1H), 3.25-3.11 (m, 1H). LC-MS (ESI, m/z): [M+H]⁺ 432.

### Example-3-3-07: (2S)-2-({[(9H-fluoren-9-yl)methoxy]carbonyl}amino)-3-(1H-indazol-5-yl)propanoic acid

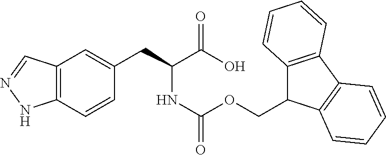 [SMILES: O=C(N[C@@H](Cc1ccc2[nH]ncc2c1)C(=O)O)OCC1c2ccccc2-c2ccccc21]

To a suspension of Zn (13.2 g, 202 mmol) in DMF (600 mL) at rt was added I₂ (2.57 g, 10.1 mmol). The mixture was stirred at rt for 2 min. To the mixture at rt was added methyl (2R)-2-{[(9H-fluoren-9-ylmethoxy)carbonyl]amino}-3-iodopropanoate (CAS: 156017-42-4, 45.7 g, 177 mmol). The mixture was stirred at rt for 1 h. To the mixture were added tert-butyl 5-bromoindazole-1-carboxylate (20.0 g, 67.3 mmol), Pd₂(dba)₃ CHCl₃ adduct (1.55 g, 1.50 mmol), and SPhos (1.39 g, 3.41 mmol). The mixture was stirred at 60° C. for 3 h, and then filtered. The filtrate was diluted with EtOAc then washed three times with H₂O. The organic extract was dried over Na₂SO₄, filtered, and then concentrated. The resulting residue was purified by silica gel flash column chromatography (isocratic, petroleum ether/EtOAc=38/62). Appropriate fractions were concentrated. To the residue (21.0 g, 38.8 mmol) at 0° C. was added 4M HCl in 1,4-dioxane (500 mL) dropwise with stirring. The resulting solution was stirred for 2 h at rt. The resulting mixture was concentrated under vacuum. To a mixture of the residue (25.0 g, 56.6 mmol) in 2-propanol (600 mL) at 0° C. were added CaCl₂ (101 g, 906 mmol) and a solution of LiOH·H₂O (9.51 g, 227 mmol) in H₂O (200 mL) dropwise. The mixture was stirred at rt for 1 h. The pH of the mixture was adjusted to pH 7 with with citric acid aq. The resulting mixture was extracted twice with EtOAc. The combined organic extracts were washed three times with H₂O, dried over Na₂SO₄, filtered, and then concentrated. The resulting residue was purified by trituration with Et₂O (500 mL) to afford the title compound (21.5 g, 50.3 mmol). ¹H NMR (400 MHz, DMSO-d₆) δ 12.97 (br, 1H), 12.75 (br, 1H), 7.98 (s, 1H), 7.92-7.84 (m, 2H), 7.82-7.72 (m, 1H), 7.68-7.57 (m, 3H), 7.53-7.35 (m, 3H), 7.34-7.16 (m, 3H), 4.28-4.06 (m, 4H), 3.24-3.11 (m, 1H), 3.03-2.88 (m, 1H). LC-MS (ESI, m/z): [M+H]⁺ 428.

The following compounds were synthesized as outlined for the preparation of Example-3-3-07 employing appropriate starting materials in the table.

| | Structure and IUPAC name |
|---|---|
| | Fmoc-protected starting material-A |
| Ex- | Starting material-B |
| ample | NMR and MS |
| 3-3-08 | 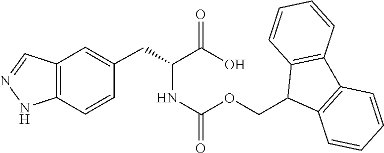 [SMILES: O=C(N[C@H](Cc1ccc2[nH]ncc2c1)C(=O)O)OCC1c2ccccc2-c2ccccc21] |
| | (2R)-2-({[(9H-fluoren-9-yl)methoxy]carbonyl} amino)-3-(1H- |
| | indazol-5-yl)propanoic acid |
| | methyl (2S)-2-{[(9H-fluoren-9-ylmethoxy)carbonyl]amino}-3- |
| | iodopropanoate (CAS: 527696-77-1) |
| | tert-butyl 5-bromoindazole-1-carboxylate |
| | ¹H NMR (400 MHZ, DMSO-d₆) δ 12.97 (br, 1H), 12.75 |
| | (br, 1H), 7.98 (s, 1H), 7.92-7.84 (m, 2H), 7.82-7.72 (m, 1H), |
| | 7.68-7.57 (m, 3H), 7.53-7.35 (m, 3H), 7.34-7.16 (m, 3H), |
| | 4.28-4.06 (m, 4H), 3.24-3.11 (m, 1H), 3.03-2.88 (m, 1H). |
| | LC-MS (ESI, m/z): [M + H]+ 428. |

### Example-3-4-01: (2S)-2-({[(9H-fluoren-9-yl)methoxy]carbonyl}(methyl)amino)-3-{1-[(prop-2-en-1-yloxy)carbonyl]piperidin-4-yl}propanoic acid

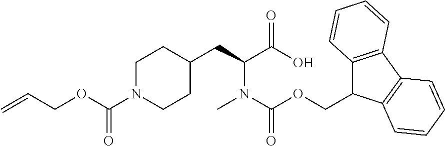 [SMILES: C=CCOC(=O)N1CCC(C[C@@H](C(=O)O)N(C)C(=O)OCC2c3ccccc3-c3ccccc32)CC1]

To a solution of (2S)-2-({[(9H-fluoren-9-yl)methoxy]carbonyl}amino)-3-{1-[(prop-2-en-1-yloxy)carbonyl]piperidin-4-yl}propanoic acid (CAS: 313052-03-8, 0.96 g, 2.00 mmol) and p-toluenesulfonic acid monohydrate (76 mg, 0.40 mmol) in toluene (20 mL) was added paraformaldehyde (300 mg, 10.0 mmol). The mixture was stirred at 100° C. for 3 h and then cooled to rt. The mixture was concentrated. The crude residue was purified by silica gel column chromatography (gradient, hexane/EtOAc=90/10 to 40/60) to afford prop-2-en-1-yl 4-{[(4S)-3-{[(9H-fluoren-9-yl)methoxy]carbonyl}-5-oxo-1,3-oxazolidin-4-yl]methyl}piperidine-1-carboxylate (1.03 g, 1.99 mmol). To a solution of the obtained compound (0.98 g, 2.00 mmol) and triisopropylsilane (3.17 g, 20 mmol) in CH₂Cl₂ (10 mL) at rt was added TFA (9.2 mL, 120 mmol). The mixture was stirred at rt for 20 h, and then concentrated. The residue was azeotroped with toluene twice. The residue was purified by silica gel column chromatography (gradient, hexane/EtOAc=90/10 to 0/100) to afford the title compound (889 mg, 1.77 mmol). ¹H NMR (300 MHz, DMSO-d₆) δ 12.8 (br, 1H), 7.91 (dd, J=7.5, 7.5 Hz, 2H), 7.71-7.55 (m, 2H), 7.49-7.23 (m, 4H), 6.22-5.82 (m, 1H), 5.48-5.07 (m, 2H), 4.90-4.11 (m, 6H), 4.11-3.75 (m, 2H), 2.96-5.58 (m, 5H), 1.84-0.48 (m, 7H). LC-MS (ESI, m/z): [M+H]⁺ 493.

### Example-3-4-02: (2S)-3-{1-[(tert-butoxy)carbonyl]piperidin-4-yl}-2-({[(9H-fluoren-9-yl)methoxy]carbonyl}(methyl)amino)propanoic acid

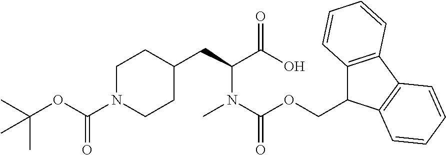 [SMILES: CN(C(=O)OCC1c2ccccc2-c2ccccc21)[C@@H](CC1CCN(C(=O)OC(C)(C)C)CC1)C(=O)O]

To a solution of Example-3-4-01 (16.5 g, 33.5 mmol) in THF (120 mL) at 0° C. were added PhSiH₃ (7.25 g, 67.0 mmol) and Pd(PPh₃)₄ (7.74 g, 6.7 mmol) at 0° C. The mixture was stirred at rt for 3 h at rt, and then added 1,4-dioxane (60 mL), H₂O (30 mL). To the mixture were added Boc₂O (8.77 g, 40.2 mmol) and NaHCO₃ (5.63 g, 67.0 mmol) at 0° C. The mixture was stirred at rt for 3 h at rt, and then quenched with critic acid aq. The mixture aqueous phase was extracted twice with EtOAc. The combined organic extracts were washed with brine, dried over Na₂SO₄ and concentrated. The residue was purified by silica gel column chromatography (gradient, hexane/EtOAc=99/1 to 50/50) and then reverse phase C18 silica gel column chromatography (gradient, H2O/MeCN with 0.1% formic acid=45/55 to 25/75) to afford the title compound (9.43 g, 18.5 mmol). ¹H NMR (500 MHz, DMSO-d₆) δ 12.8 (br, 1H), 8.09-7.82 (m, 2H), 7.74-7.52 (m, 2H), 7.52-7.21 (m, 4H), 4.85-4.16 (m, 4H), 3.96-3.68 (m, 2H), 2.94-2.36 (m, 5H), 1.86-0.36 (m, 16H) LC-MS (ESI, m/z): [M+H]⁺ 409.

### Example-3-4-03: (2S)-3-(1-acetylpiperidin-4-yl)-2-({[(9H-fluoren-9-yl)methoxy]carbonyl}(methyl)amino)propanoic acid

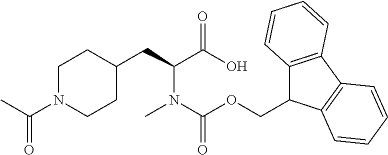 [SMILES: CC(=O)N1CCC(C[C@@H](C(=O)O)N(C)C(=O)OCC2c3ccccc3-c3ccccc32)CC1]

To a solution of Example-3-4-02 (5.00 g, 9.83 mmol) in CH₂Cl₂ (49 mL) at 0° C. was added 4M hydrogen chloride in MTHP (9.8 mL, 39.3 mmol). The mixture was stirred at rt for 2 h. The resulting solid was collected by filtration to afford (2S)-2-({[(9H-fluoren-9-yl)methoxy]carbonyl}(methyl)amino)-3-(piperidin-4-yl)propanoic acid hydrochloride (3.27 g, 7.28 mmol). To a solution of the resulting residue (1.00 g, 2.25 mmol) in 1,4-dioxane (10 mL) and H₂O (10 mL) at 0° C. was added NaHCO₃ (755 mg, 8.99 mmol), followed by Ac₂O (241 mg, 2.36 mmol) dropwise. The mixture was stirred at 0° C. for 0.5 h, and then pH of the solution was adjusted to 4 with 2M aq. HCl at 0° C. The mixture was extracted with EtOAc. The organic extracts were washed with brine, dried over Na₂SO₄, filtered, and concentrated. The resulting residue was azeotroped with toluene and purified by silica gel flash column chromatography (gradient, CH₂Cl₂/MeOH=100/0 to 80/20) to afford the title compound (776 mg, 1.69 mmol). ¹H NMR (500 MHz, DMSO-d₆) δ 12.8 (br, 1H), 8.05-7.78 (m, 2H), 7.52-7.17 (m, 2H), 7.52-7.17 (m, 4H), 4.87-4.15 (m, 5H), 3.91-3.52 (m, 1H), 4.51-4.15 (m, 4H), 2.48-2.15 (m, 1H), 2.12-1.85 (m, 3H), 1.85-0.55 (m, 7H). LC-MS (ESI, m/z): [M+H]⁺ 451.

### Intermediate-3-4-01. prop-2-en-1-yl (2S)-2-({[(9H-fluoren-9-yl)methoxy]carbonyl}(methyl)amino)-3-(piperidin-4-yl)propanoate hydrochloride

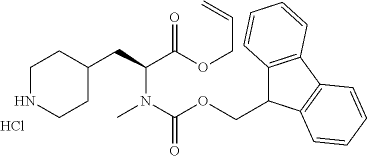 [SMILES: Cl ; C=CCOC(=O)[C@H](CC1CCNCC1)N(C)C(=O)OCC1c2ccccc2-c2ccccc21]

To a solution of Example-3-4-02 (4.05 g, 7.96 mmol) in DMF (19.9 mL) at 0° C. were added DIPEA (2.09 mL, 11.9 mmol) and allylbromide (0.76 mL, 8.76 mmol). The mixture was stirred 0° C. for 0.5 h and at rt for 2 h. To the mixture at rt was added allylbromide (0.28 mL. 3.18 mmol). The reaction was stirred at rt for 3 h. The reaction was diluted with EtOAc and washed with 1M aq. HCl. The aqueous phase was extracted with EtOAc. The combined organic phase was washed with satd. aq NaHCO₃, water and brine, dried over Na₂SO₄, filtered, and concentrated. The resulting residue was purified by silica gel flash column chromatography (gradient, hexane/EtOAc=100/0 to 25/75). Appropriate fractions were concentrated. To the resulting residue (3.78 g, 6.89 mmol) in CH₂Cl₂ (11.5 mL) at 0° C. was added 4M hydrogen chloride in MTHP (11.5 mL, 45.9 mmol). The mixture was stirred at rt for 2 h and then concentrated. The resulting residue was triturated with hexane. The resulting solid was collected by filtration to afford the title compound (3.14 g, 6.47 mmol). LC-MS (ESI, m/z): [M+H]⁺ 449.

### Example-3-4-04: (2S)-2-({[(9H-fluoren-9-yl)methoxy]carbonyl}(methyl)amino)-3-{1-[1-(triphenylmethyl)-1H-pyrazole-4-carbonyl]piperidin-4-yl}propanoic acid

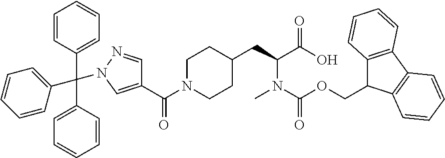 [SMILES: CN(C(=O)OCC1c2ccccc2-c2ccccc21)[C@@H](CC1CCN(C(=O)c2cnn(C(c3ccccc3)(c3ccccc3)c3ccccc3)c2)CC1)C(=O)O]

To a solution of Intermediate-3-4-01 (1.03 g, 2.12 mmol) and 1-(triphenylmethyl)-1H-pyrazole-4-carboxylic acid (CAS: 875554-07-7, 826 mg, 2.33 mmol) in THF (4.24 mL) at 0° C. were added EDCI·HCl (447 mg, 2.33 mmol), DIPEA (0.37 mL, 2.12 mmol) and Oxyma Pure (CAS: 3849-21-6, 331 mg, 2.33 mmol). The mixture was stirred at rt for 19 h, and then diluted with EtOAc. The mixture was washed with 1M aq. HCl, satd. aq NaHCO₃, water and brine, dried over Na₂SO₄, filtered, and concentrated. The resulting residue was purified by silica gel flash column chromatography (gradient, hexane/EtOAc=100/0 to 0/100) to afford the title compound (1.60 g, 2.04 mmol). ¹H NMR (500 MHz, DMSO-d₆) δ 12.8 (br, 1H), 7.91-7.82 (m, 3H), 7.70-7.05 (m, 22H), 4.34-3.76 (m, 6H), 3.14-2.45 (m, 5H), 1.79-0.77 (m, 7H). LC-MS (ESI, m/z): [M+H]⁺ 745.

The following compounds were synthesized as outlined for the preparation of Example-3-4-04 employing appropriate starting materials in the table.

| | Structure and IUPAC name |
|---|---|
| | Fmoc-protected starting material-A |
| | Starting material-B |
| Example | NMR and MS |
| 3-4-05 | 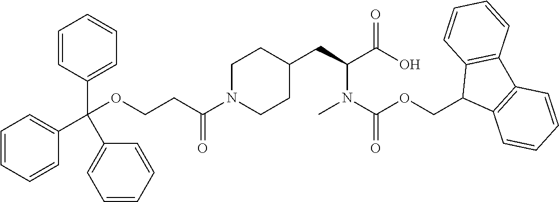 [SMILES: CN(C(=O)OCC1c2ccccc2-c2ccccc21)[C@@H](CC1CCN(C(=O)CCOC(c2ccccc2)(c2ccccc2)c2ccccc2)CC1)C(=O)O] |
| | (2S)-2-({[(9H-fluoren-9-yl)methoxy]carbonyl}(methyl)amino)-3-{1-[3- |
| | (triphenyl methoxy)propanoyl]piperidin-4-yl}propanoic acid |
| | Intermediate-3-4-01 |
| | 3-(triphenylmethoxy)propanoic acid (CAS: 24243-01-4) |
| | ¹H NMR (500 MHz, DMSO-d₆) δ 12.8 (br, 1H), 7.93-7.84 (m, 2H), 7.68- |
| | 7.21 (m, 21H), 4.69-4.16 (m, 5H), 3.90-3.64 (m, 1H), 3.27-3.12 (m, |
| | 2H), 3.00-2.08 (m, 7H), 1.86-0.47 (m, 7H). LC-MS (ESI, m/z): not |
| | found. |
| 3-4-06 | 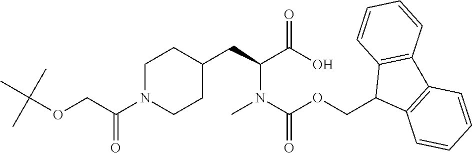 [SMILES: CN(C(=O)OCC1c2ccccc2-c2ccccc21)[C@@H](CC1CCN(C(=O)COC(C)(C)C)CC1)C(=O)O] |
| | (2S)-3-{1-[2-(tert-butoxy)acetyl]piperidin-4-yl}-2-({[(9H-fluoren-9- |
| | yl)methoxy]carbonyl}(methyl)amino)propanoic acid |
| | Intermediate-3-4-01 |
| | 2-(tert-butoxy)acetic acid (CAS: 13211-32-0) |
| | ¹H NMR (500 MHz, DMSO-d₆) δ 12.9 (br, 1H), 7.94-7.81 (m, 2H), 7.69- |
| | 7.53 (m, 2H), 7.46-7.27 (m, 4H), 4.76-4.16 (m, 5H), 4.02-3.66 (m, 3H), |
| | 2.99-2.15 (m, 5H), 1.79-0.77 (m, 16H). LC-MS (ESI, m/z): [M + H]⁺ 523. |

### Example-3-4-07: (2S)-2-({[(9H-fluoren-9-yl)methoxy]carbonyl}amino)-7-methoxyheptanoic acid

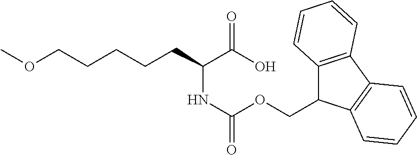 [SMILES: COCCCCC[C@H](NC(=O)OCC1c2ccccc2-c2ccccc21)C(=O)O]

To a mixture of (15S)-9-chloro-19-[(3,4-dichlorophenyl)methyl]-6-phenyl-2-oxa-5lambda4,13,19lambda4-triaza-1-nickelapentacyclo[11.6.0.01,5.07,12.015,19]nonadeca-5,7(12),8,10-tetraene-3,14-dione (CAS: 2089661-84-5, 180 g, 300 mmol) and 1-bromo-5-methoxypentane (CAS: 14155-86-3, 108 g, 598 mmol) in DMF (1500 mL) at 0° C. was added 30% NaOMe in MeOH (64.7 g, 359 mmol) dropwise. The mixture was stirred at rt for 2 h. The reaction was quenched with H₂O. The mixture was stirred at rt for 0.5 h, and then diluted with H₂O. After 48 h, the solids formed were collected by filtration. Then the solids were washed with water and dried in an oven under reduced pressure. To the resulting solid (150 g, 214 mmol) in DME (750 mL) was added 3M HCl aq. (345 mL). The mixture was stirred at 60° C. for 3 h. The resulting mixture was concentrated under vacuum. Then the mixture was washed with H₂O (600 mL) and the solids were filtered out. To the aqueous phase were added MeCN (600 mL) and edta disodium salt dihydrate (79.4 g, 214 mmol). The mixture was stirred at 0° C. for 0.5 h. To the mixture were added NaOH (35.1 g, 876 mmol) in portions at 0° C. To the mixture were added Na₂CO₃ (29.5 g, 278 mmol) and Fmoc-OSu (72.1 g, 214 mmol) at 0° C. The mixture was stirred for 2 h at rt, and then pH of the solution was adjusted to 4 with 3 M aq. HCl. The mixture was diluted with water and extracted three times with EtOAc. The organic extracts were washed three times with brine, dried over Na₂SO₄, filtered, and concentrated. The resulting residue was purified by silica gel flash column chromatography (isocratic, CH₂Cl₂/MeOH=91/9) and then further purified by reversed phase silica gel flash column chromatography (C18 silica gel, gradient, H₂O/CH₃CN with 0.1% formic acid=60/40 to 40/60) to afford the title compound (49.6 g, 125 mmol). ¹H NMR (400 MHz, DMSO-d₆) δ 12.5 (br, 1H), 7.90 (d, J=7.5 Hz, 2H), 7.73 (d, J=7.4 Hz, 2H), 7.64 (d, J=8.0 Hz, 1H), 7.52-7.38 (m, 2H), 7.38-7.19 (m, 2H), 4.42-4.09 (m, 3H), 4.05-3.78 (m, 1H), 3.55-3.06 (m, 7H), 1.83-1.41 (m, 4H), 1.40-1.11 (m, 4H). LC-MS (ESI, m/z): [M+H]⁺ 398.

### Example-3-4-08: (2S)-3-[3′-(tert-butoxy)-[1,1′-biphenyl]-4-yl]-2-({[(9H-fluoren-9-yl)methoxy]carbonyl}amino)propanoic acid

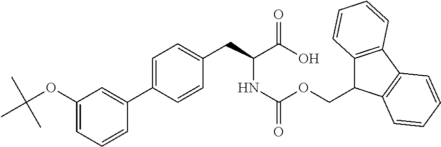 [SMILES: CC(C)(C)Oc1cccc(-c2ccc(C[C@H](NC(=O)OCC3c4ccccc4-c4ccccc43)C(=O)O)cc2)c1]

To a suspension of Zn (28.0 g, 428 mmol) in DMF (1000 mL) was added I₂ (5.4 g, 21.2 mmol). The mixture was stirred at rt for 10 min. To the mixture at rt was added methyl (2S)-2-[[(9H-fluoren-9-ylmethoxy)carbonyl]amino]-3-iodopropanoate (CAS: 527696-77-1, 64.0 g, 142 mmol). The mixture was stirred at rt for an additional 1 h. To the mixture were added 4′-bromo-3-methoxy-1,1′-biphenyl (CAS: 74447-69-1, 42.5 g, 140 mmol), Pd₂(dba)₃ CHCl₃ adduct (4.40 g, 4.25 mmol), and SPhos (2.92 g, 7.11 mmol). The mixture was stirred at 50° C. for an additional 1 h, and then filtered. The filtrate was diluted with H₂O. The mixture was extracted three times with EtOAc. The combined organic extracts were washed with brine, dried over Na₂SO₄, filtered, and then concentrated. The resulting residue was purified by silica gel flash column chromatography (gradient, petroleum ether/EtOAc=100/1 to 1/1). Appropriate fractions were concentrated (46.0 g, 83.7 mmol). To a mixture of the obtained material (30.0 g, 54.6 mmol) in 2-propanol (450 mL) and H₂O (150 mL) at 0° C. were added CaCl₂ (96.9 g, 873 mmol) and LiOH·H₂O (5.23 g, 218 mmol). The mixture was stirred at rt for 3 h. The pH of the mixture was adjusted to pH 7 with aq. citric acid. The resulting mixture was extracted three times with EtOAc. The combined organic extracts were washed three times with H₂O, dried over Na₂SO₄, filtered, and then concentrated. The resulting residue was purified by reversed phase silica gel flash column chromatography (C18 silica gel, gradient, H₂O/CH₃CN with 10 mM NH₄HCO₃=65/35 to 35/65) to afford the title compound (8.01 g, 14.9 mmol). ¹H NMR (400 MHz, DMSO-d₆) δ 12.6 (br, 1H), 7.68 (d, J=7.6 Hz, 2H), 7.59 (d, J=8.4 Hz, 1H), 7.51-7.25 (m, 4H), 7.25-6.93 (m, 9H), 6.83-6.69 (m, 1H), 4.13-3.89 (m, 4H), 2.99-2.81 (m, 1H), 2.81-2.61 (m, 1H), 1.12 (s, 9H).

LC-MS (ESI, m/z): [M+H]⁺ 536.

### Intermediate-3-5-01: 2-[(4S)-3-{[(9H-fluoren-9-yl)methoxy]carbonyl}-5-oxo-1,3-oxazolidin-4-yl]acetic acid

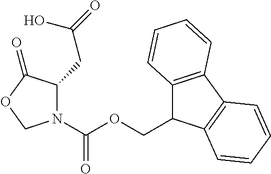 [SMILES: O=C(O)C[C@H]1C(=O)OCN1C(=O)OCC1c2ccccc2-c2ccccc21]

To a solution of (2S)-2-({[(9H-fluoren-9-yl)methoxy]carbonyl}amino)butanedioic acid (CAS: 119062-05-4, 6.00 g, 16.9 mmol) in toluene (100 mL) at rt were added CSA (0.24 g, 1.01 mmol) and paraformaldehyde (2.53 g, 84.0 mmol). The mixture was stirred at 100° C. for 1 h, and then filtered. The filtrate was diluted with EtOAc and washed with H₂O and brine, dried over Na₂SO₄, filtered and concentrated to afford the title compound (6.00 g, 16.3 mmol). LC-MS (ESI, m/z): [M+H]⁺ 368.

### Intermediate-3-5-02: 2-[(4R)-3-{[(9H-fluoren-9-yl)methoxy]carbonyl}-5-oxo-1,3-oxazolidin-4-yl]acetic acid

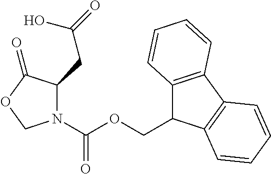 [SMILES: O=C(O)C[C@@H]1C(=O)OCN1C(=O)OCC1c2ccccc2-c2ccccc21]

To a solution of (2R)-2-({[(9H-fluoren-9-yl)methoxy]carbonyl}amino)butanedioic acid (CAS: 136083-57-3, 8.08 g, 22.8 mmol) in toluene (56.9 mL) at rt were added CSA (0.26 g, 1.14 mmol) and paraformaldehyde (3.42 g, 114 mmol). The mixture was stirred at 100° C. for 1 h, and then filtered. The filtrate was diluted with EtOAc and washed with H₂O and brine, dried over Na₂SO₄, filtered and concentrated. The resulting residue was purified by silica gel flash column chromatography (gradient, CH₂Cl₂/MeOH=100/0 to 90/10) to afford the title compound ((8.00 g, 21.8 mmol). LC-MS (ESI, m/z): [M+H]⁺ 368.

### Example-3-5-04: (2S)-2-({[(9H-fluoren-9-yl)methoxy]carbonyl}amino)-4-methanesulfonamidobutanoic acid

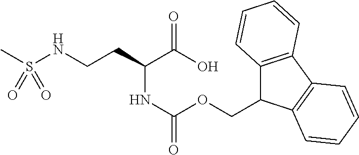 [SMILES: CS(=O)(=O)NCC[C@H](NC(=O)OCC1c2ccccc2-c2ccccc21)C(=O)O]

To a suspension of (2S)-4-amino-2-({[(9H-fluoren-9-yl)methoxy]carbonyl}amino)butanoic acid (CAS: 161420-87-7, 200 g, 588 mmol) in THF (2000 mL) at 0° C. was added BSA (538 g, 2.64 mol) then stirred at rt for 2 h. To the mixture was at 0° C. was added dropwise methanesulfonyl chloride (CAS: 124-63-0, 80.8 g, 705 mmol). The resulting mixture was stirred at rt for further 3 h then the pH of the solution was adjusted to 8 with satd aq. NaHCO₃ at 0° C. The mixture was stirred at rt for 1 h. The pH of the solution was adjusted to 5 with 2M aq. HCl at 0° C. The mixture was extracted three times with EtOAc. The combined organic extracts were washed with brine, dried over Na₂SO₄, filtered, and concentrated. The resulting residue was purified by silica gel flash column chromatography (isocratic, CH₂Cl₂/MeOH=83/17) to afford the title compound (114 g, 272 mmol). ¹H NMR (400 MHz, DMSO-d₆) δ 12.7 (br, 1H), 7.89 (d, J=7.6 Hz, 2H), 7.77-7.63 (m, 3H), 7.46-7.28 (m, 4H), 7.12-7.00 (m, 1H), 4.35-4.18 (m, 3H), 4.11-4.00 (m, 1H), 3.11-2.97 (m, 2H), 2.89 (s, 3H), 2.04-1.71 (m, 2H). LC-MS (ESI, m/z): [M+H]⁺ 419.

### Example-3-5-05: (2R)-2-({[(9H-fluoren-9-yl)methoxy]carbonyl}(methyl)amino)-3-{[(prop-2-en-1-yloxy)carbonyl]amino}propanoic acid

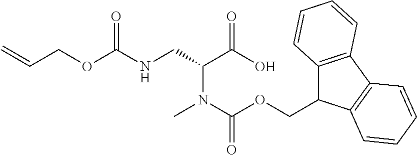 [SMILES: C=CCOC(=O)NC[C@H](C(=O)O)N(C)C(=O)OCC1c2ccccc2-c2ccccc21]

To a solution of Intermediate-3-5-02 (72.8 g, 198 mmol) in THF (800 mL) at −15° C. under nitrogen atmosphere were added N-methylmorpholine (CAS: 109-02-4, 22.1 g, 218 mmol) and isobutyl chloroformate (CAS: 543-27-1, 29.8 g, 218 mmol). The mixture was stirred at −15° C. for 15 min. To the mixture at 0° C. was added dropwise a solution of sodium azide (CAS: 26628-22-8, 19.3 g, 296 mmol) in H₂O (80 mL). The resulting mixture was stirred at rt for 2 h then diluted with H₂O. The mixture was extracted twice with EtOAc and the combined organic extracts were washed with H₂O and 1M aq. HCl then washed twice with satd. aq. NaHCO₃ and brine, dried over Na₂SO₄, filtered and concentrated. To the resulting residue (55.0 g, 140 mmol) in toluene (600 mL) at rt was added t-Butanol (CAS: 75-65-0, 260 g, 3.50 mol). The mixture was stirred at 90° C. for 2 h then concentrated. The resulting residue was purified by silica gel flash column chromatography (isocratic, petroleum ether/EtOAc=88/12). Appropriate fractions were collected and concentrated. To the resulting residue (45.0 g, 103 mmol) in CHCl₃ (400 mL) at 0° C. were added triethylsilane (CAS: 617-86-7, 35.8 g, 308 mmol) and 2,2,2-trifluoroacetic acid (400 mL, 5.25 mol). The mixture was stirred at 60° C. for 24 h and then concentrated. To the resulting residue (50.7 g, 112 mmol) in 1,4-dioxane (600 mL) and H₂O (200 mL) at 0° C. were added of NaHCO₃ (28.2 g, 335 mmol) and 2,5-dioxopyrrolidin-1-yl prop-2-en-1-yl carbonate (CAS: 135544-68-2, 19.6 g, 106 mmol). The mixture was stirred at rt for 3 h. The mixture was filtered then pH of the filtrate was adjusted to 4 at 0° C. with 1M aq. HCl. The mixture was extracted three times with EtOAc and the combined organic extracts were washed with brine, dried over Na₂SO₄, filtered and concentrated. The resulting residue was purified by reversed phase silica gel flash column chromatography (C18 silica gel, gradient, H₂O with 0.1% formic acid/CH₃CN=90/10 to 0/100) to afford the title compound (23.3 g, 54.8 mmol). ¹H NMR (400 MHz, DMSO-d₆) δ 13.1 (br, 1H), 7.95-7.85 (m, 2H), 7.72-7.56 (m, 2H), 7.52-7.25 (m, 5H), 5.97-5.75 (m, 1H), 5.34-5.04 (m, 2H), 4.89-4.58 (m, 1H), 4.53-4.39 (m, 2H), 4.34-4.14 (m, 3H), 3.73-3.24 (m, 2H), 2.92-2.79 (m, 3H). LC-MS (ESI, m/z): [M+H]⁺ 425.

### Example-3-5-06: (2S)-3-{[(tert-butoxy)carbonyl]amino}-2-({[(9H-fluoren-9-yl)methoxy]carbonyl}(methyl)amino)propanoic acid

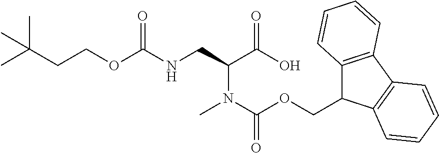 [SMILES: CN(C(=O)OCC1c2ccccc2-c2ccccc21)[C@@H](CNC(=O)OCCC(C)(C)C)C(=O)O]

To a solution of (9H-fluoren-9-yl)methyl (4S)-4-({[(tert-butoxy)carbonyl]amino}methyl)-5-oxo-1,3-oxazolidine-3-carboxylate (CAS: 918428-69-0, 4.00 g, 9.12 mmol) in CHCl₃ (50.0 mL) at 0° C. were added triethylsilane (CAS: 617-86-7, 3.19 g, 27.4 mmol) and TFA (50.0 mL, 656 mmol). The mixture was stirred at 70° C. for 24 h and then concentrated. To the obtained material (6.89 g, 11.7 mmol) in THF (90 mL) at rt were added a solution of Na₂CO₃ (2.49 g, 23.5 mmol) in H₂O (90.0 mL) and di-tert-butyl dicarbonate (CAS: 24424-99-5, 3.84 g, 17.6 mmol). The mixture was stirred at rt for 3 h. The pH of the mixture was adjusted to 7 with 5M aq. citric acid at 0° C. The mixture was extracted three times with EtOAc and the combined organic extracts were washed with brine, dried over Na₂SO₄, filtered and concentrated. The resulting residue was purified by reversed phase silica gel flash column chromatography (C18 silica gel, gradient, H₂O/CH₃CN=80/20 to 60/40) to afford the title compound (3.08 g, 6.99 mmol). ¹H NMR (300 MHz, Methanol-d₄) δ 7.85-7.77 (m, 2H), 7.68-7.59 (m, 2H), 7.45-7.27 (m, 4H), 4.82-4.66 (m, 1H), 4.52-4.20 (m, 3H), 3.69-3.35 (m, 2H), 2.93 (s, 3H), 1.50-1.35 (m, 9H). LC-MS (ESI, m/z): [M−H]⁻ 439.

### Example-3-6-01: (2S)-2-({[(9H-fluoren-9-yl)methoxy]carbonyl}amino)-3-{3-[2-oxo-2-(prop-2-en-1-yloxy)ethyl]phenyl}propanoic acid

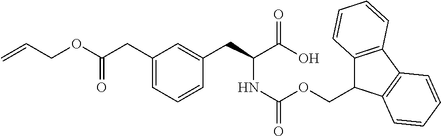 [SMILES: C=CCOC(=O)Cc1cccc(C[C@H](NC(=O)OCC2c3ccccc3-c3ccccc32)C(=O)O)c1]

To a suspension of Zn (5.43 g, 83.02 mmol) in DMF (150 mL) was added I₂ (1.05 g, 4.14 mmol). The mixture was stirred at rt for 30 min. To the mixture at rt was added methyl (2R)-2-[[(9H-fluoren-9-ylmethoxy)carbonyl]amino]-3-iodopropanoate (CAS: 156017-42-4, 12.48 g, 27.66 mmol). The mixture was stirred at rt for an additional 1 h. To the mixture were added tert-butyl 2-(3-bromophenyl)acetate (CAS: 197792-52-2, 7.50 g, 27.66 mmol), Pd₂(dba)₃ CHCl₃ adduct (0.86 g, 0.83 mmol), and SPhos (0.57 g, 1.38 mmol). The mixture was stirred at 50° C. for an additional 3 h, and then filtered. The filtrate was diluted with H₂O. The mixture was extracted three times with EtOAc. The combined organic extracts were washed three times with brine, dried over Na₂SO₄, filtered, and then concentrated. The resulting residue was purified by silica gel flash column chromatography (gradient, petroleum ether/EtOAc=99/1 to 89/11). Appropriate fractions were concentrated. To a solution of the resulting residue in DCM (100 mL) was added TFA (50.00 mL). The resulting solution was stirred at room temperature for 2 h. The resulting mixture was concentrated. To a solution of the resulting residue (5.35 g, 11.64 mmol) in MeCN (50.00 mL) was added DIPEA (2.26 g, 17.47 mmol), and allyl bromide (CAS: 106-95-6, 1.41 g, 11.64 mmol). The resulting solution was stirred at room temperature for 16 h. The reaction was quenched by the addition of 40 mL of water. The mixture was extracted twice with EtOAc. The combined organic extracts were washed with brine, dried over Na₂SO₄, filtered, and then concentrated. The resulting residue was purified by silica gel flash column chromatography (gradient, petroleum ether/EtOAc=99/1 to 75/25). Appropriate fractions were concentrated and combined with other several batches. To a solution of the resulting residue (80.00 g, 160.14 mmol) in ClCH₂CH₂Cl (800 mL) was added Me₃SnOH (57.68 g, 318.68 mmol). The resulting mixture was stirred at 40° C. for 16 h. The reaction was quenched with aq. citric acid. The mixture was extracted three times with EtOAc. The combined organic extracts were washed with brine, dried over Na₂SO₄, filtered, and then concentrated. The resulting residue was purified by silica gel flash column chromatography (DCM/MeOH=90/10) to afford the title compound (50.72 g, 5.79 mmol). ¹H NMR (300 MHz, DMSO-d₆) δ 12.75 (br, 1H), 7.88 (d, J=7.4 Hz, 2H), 7.74 (d, J=8.4 Hz, 1H), 7.70-7.61 (m, 2H), 7.44-7.37 (m, 2H), 7.36-7.07 (m, 6H), 5.98-5.78 (m, 1H), 5.33-5.11 (m, 2H), 4.61-4.43 (m, 2H), 4.30-4.09 (m, 4H), 3.67 (s, 2H), 3.14-2.98 (m, 1H), 2.95-2.78 (m, 1H). LC-MS (ESI, m/z): [M+H]⁺ 486.

The following compounds were synthesized as outlined for the preparation of Example-3-6-01 employing appropriate starting materials in the table.

| | Structure and IUPAC name |
|---|---|
| | Fmoc-protected starting material-A |
| | Starting material-B |
| Example | NMR and MS |
| 3-6-02 | 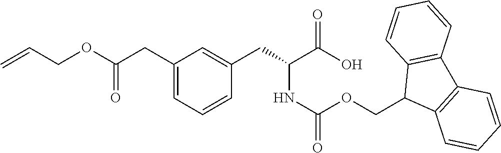 [SMILES: C=CCOC(=O)Cc1cccc(C[C@@H](NC(=O)OCC2c3ccccc3-c3ccccc32)C(=O)O)c1] |
| | (2R)-2-({[(9H-fluoren-9-yl)methoxy]carbonyl}amino)-3-{3-[2-oxo-2- |
| | (prop-2-en-1-yloxy)ethyl]phenyl}propanoic acid |
| | methyl (2S)-2-[[(9H-fluoren-9-ylmethoxy)carbonyl]amino]-3- |
| | iodopropanoate (CAS: 527696-77-1) |
| | tert-butyl 2-(3-bromophenyl)acetate (CAS: 197792-52-2) |
| | Substantially identical to Example-3-6-01 |

### Example-3-6-03: (2S)-3-{3-[2-(tert-butoxy)-2-oxoethyl]phenyl}-2-({[(9H-fluoren-9-yl)methoxy]carbonyl}amino)propanoic acid

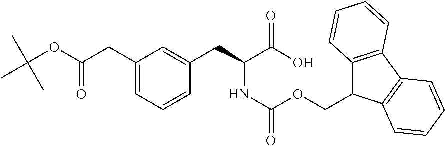 [SMILES: CC(C)(C)OC(=O)Cc1cccc(C[C@H](NC(=O)OCC2c3ccccc3-c3ccccc32)C(=O)O)c1]

To a suspension of Zn (7.23 g, 110.64 mmol) in DMF (1800 mL) ar rt was added I₂ (2.81 g, 11.06 mmol). The mixture was stirred at rt for 10 min. To the mixture at rt was added methyl (2R)-2-[[(9H-fluoren-9-ylmethoxy)carbonyl]amino]-3-iodopropanoate (CAS: 156017-42-4, 19.97 g, 44.26 mmol). The mixture was stirred at rt for an additional 30 min. To the mixture were added tert-butyl 2-(3-bromophenyl)acetate (CAS: 197792-52-2, 10 g, 36.88 mmol), Pd₂(dba)₃ CHCl₃ adduct (1.69 g, 1.84 mmol), and SPhos (3.03 g, 7.38 mmol). The mixture was stirred at 50° C. for 30 min. Then the mixture was stirred at 60° C. for 2.5 h. The mixture was filtered. The filtrate was diluted with H₂O. The mixture was extracted three times with EtOAc. The combined organic extracts were washed three times with brine, dried over Na₂SO₄, filtered, and then concentrated. The resulting residue was purified by silica gel flash column chromatography (gradient, petroleum ether/EtOAc=90/10 to 70/30). Appropriate fractions were concentrated. Then the resulting residue was purified by reversed phase silica gel flash column chromatography (C18 silica gel, gradient, H₂O with 0.1% formic acid/CH₃CN with 0.1% formic acid=40/60 to 10/90). Appropriate fractions were concentrated and combined with other several batches. To a solution of the resulting residue (60.00 g, 116.37 mmol) in 2-propanol (1800 mL) and H₂O (600 mL), THF (402 mL) at 0° C. were added CaCl₂ (206.64 g, 1861.89 mmol) and LiOH·H₂O (19.53 g, 465.47 mmol). The mixture was stirred at rt for 2 h, and then pH of the solution was adjusted to 5 with sodium dihydrogen phosphate aqueous solution. The resulting mixture was extracted three times with EtOAc. The combined organic extracts were washed three times with brine, dried over Na₂SO₄, filtered, and then concentrated. The resulting residue was purified by silica gel flash column chromatography (gradient, petroleum ether/EtOAc=80/20 to 40/60) to afford the title compound (52.92 g, 105.42 mmol). ¹H NMR (300 MHz, DMSO-d₆) δ 12.75 (br, 1H), 7.88 (d, J=7.5 Hz, 2H), 7.75 (d, J=8.3 Hz, 1H), 7.71-7.61 (m, 2H), 7.45-7.36 (m, 2H), 7.35-7.05 (m, 6H), 4.19 (br, 4H), 3.49 (s, 2H), 3.14-3.00 (m, 1H), 2.97-2.77 (m, 1H), 1.38 (s, 9H). LC-MS (ESI, m/z): [M+H]⁺ 502.

The following compounds were synthesized as outlined for the preparation of Example-3-6-03 employing appropriate starting materials in the table.

| | Structure and IUPAC name |
|---|---|
| | Fmoc-protected starting material-A |
| | Starting material-B |
| Example | NMR and MS |
| 3-6-04 | 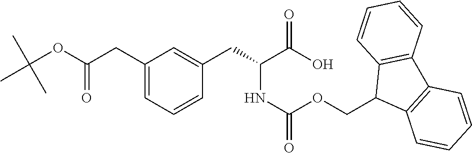 [SMILES: CC(C)(C)OC(=O)Cc1cccc(C[C@@H](NC(=O)OCC2c3ccccc3-c3ccccc32)C(=O)O)c1] |
| | (2R)-3-{3-[2-(tert-butoxy)-2-oxoethyl]phenyl}-2-({[(9H-fluoren-9- |
| | yl)methoxy]carbonyl}amino)propanoic acid |
| | methyl (2S)-2-[[(9H-fluoren-9-ylmethoxy)carbonyl]amino]-3- |
| | iodopropanoate (CAS: 527696-77-1) |
| | tert-butyl 2-(3-bromophenyl)acetate (CAS: 197792-52-2) |
| | Substantially identical to Example-3-6-03 |
| 3-6-05 | 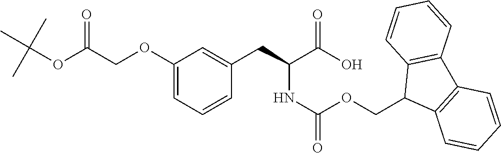 [SMILES: CC(C)(C)OC(=O)COc1cccc(C[C@H](NC(=O)OCC2c3ccccc3-c3ccccc32)C(=O)O)c1] |
| | (2S)-3-{3-[2-(tert-butoxy)-2-oxoethoxy]phenyl}-2-({[(9H-fluoren-9- |
| | yl)methoxy]carbonyl}amino)propanoic acid |
| | methyl (2R)-2-[[(9H-fluoren-9-ylmethoxy)carbonyl]amino]-3- |
| | iodopropanoate (CAS: 156017-42-4) |
| | tert-butyl 2-(3-iodophenoxy)acetate (CAS: 474009-89-7) |
| | ¹H NMR (400 MHZ, DMSO-d₆) δ 12.78 (br, 1H), 7.88 (d, J = 7.5 Hz, 2H), |
| | 7.75 (d, J = 8.5 Hz, 1H), 7.69-7.57 (m, 2H), 7.44-7.38 (m, 2H), 7.35- |
| | 7.26 (m, 2H), 7.24-7.15 (m, 1H), 6.93-6.85 (m, 2H), 6.81-6.68 (m, 1H), |
| | 4.60 (s, 2H), 4.36-4.11 (m, 4H), 3.13-3.07 (m, 1H), 2.93-2.78 (m, 1H), |
| | 1.42 (s, 9H). LC-MS (ESI, m/z): [M − H]⁻ 516. |
| 3-6-06 | 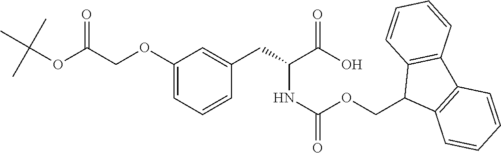 [SMILES: CC(C)(C)OC(=O)COc1cccc(C[C@@H](NC(=O)OCC2c3ccccc3-c3ccccc32)C(=O)O)c1] |
| | (2R)-3-{3-[2-(tert-butoxy)-2-oxoethoxy]phenyl}-2-({[(9H-fluoren-9- |
| | yl)methoxy]carbonyl}amino)propanoic acid |
| | methyl (2S)-2-[[(9H-fluoren-9-ylmethoxy)carbonyl]amino]-3- |
| | iodopropanoate (CAS: 527696-77-1) |
| | tert-butyl 2-(3-bromophenoxy)acetate (CAS: 277331-38-1) |
| | Substantially identical to Example-3-6-05 |

### Example-3-6-07: (2S)-2-({[(9H-fluoren-9-yl)methoxy]carbonyl}amino)-3-{3-[2-oxo-2-(prop-2-en-1-yloxy)ethoxy]phenyl}propanoic acid

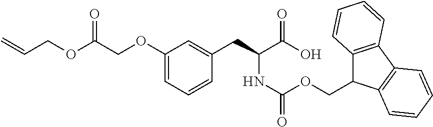 [SMILES: C=CCOC(=O)COc1cccc(C[C@H](NC(=O)OCC2c3ccccc3-c3ccccc32)C(=O)O)c1]

To a suspension of Zn (1.76 g, 26.94 mmol) in DMF (50 mL) was added I₂ (0.51 g, 2.02 mmol). The mixture was stirred at rt for 10 min. To the mixture at rt was added tert-butyl (2R)-2-{[(tert-butoxy)carbonyl]amino}-3-iodopropanoate (CAS: 1057341-65-7, 5.00 g, 13.47 mmol). The mixture was stirred at rt for an additional 1 h. To the mixture at rt were added 3-iodophenol (CAS: 626-02-8, 3.56 g, 16.18 mmol), Pd₂(dba)₃ CHCl₃ adduct (0.31 g, 0.34 mmol), and SPhos (0.55 g, 1.35 mmol). The mixture was stirred at 50° C. for 16 h, and then filtered. The filtrate was diluted with EtOAc. The mixture was washed twice with brine, dried over Na₂SO₄, filtered, and then concentrated. The resulting residue was purified by silica gel flash column chromatography (petroleum ether/EtOAc=75/25). Appropriate fractions were concentrated and combined with other several batches. To the solution of resulting residue (13.08 g, 38.77 mmol) in MeCN (150 mL) were added prop-2-en-1-yl 2-chloroacetate (CAS: 29816-14-5, 7.82 g, 58.15 mmol), and K₂CO₃ (10.79 g, 77.53 mmol). The mixture was stirred at 50° C. for 3 days. The mixture was filtered. The filtrate was concentrated. The resulting residue was purified by silica gel flash column chromatography (petroleum ether/EtOAc=85/15). Appropriate fractions were concentrated. To the solution of resulting residue (11.30 g, 25.95 mmol) in DCM (50.00 mL) at rt was added TFA (50.00 mL). The resulting solution was stirred at rt for 12 hr. The mixture was concentrated. To a solution of the resulting residue (8.00 g, 20.34 mmol) in 1,4-dioxane (60 mL) and H₂O (40 mL) at 0° C. was added NaHCO₃ (3.42 g, 40.68 mmol), followed by a solution of Fmoc-OSu (7.20 g, 21.36 mmol) in 1,4-dioxane (40 mL). The mixture was stirred at rt for 16 h, and then pH of the solution was adjusted to 4 with aq. citric acid at 0° C. The mixture was extracted three times with EtOAc. The combined organic extracts were dried over Na₂SO₄, filtered, and then concentrated. The resulting residue was purified by silica gel flash column chromatography (isocratic, CH₂Cl₂/MeOH=90/10) to afford the title compound (2.55 g, 5.08 mmol). ¹H NMR (400 MHz, DMSO-d₆) δ 12.79 (s, 1H), 7.89 (d, J=7.6 Hz, 2H), 7.76 (d, J=8.3 Hz, 1H), 7.69-7.61 (m, 2H), 7.45-7.37 (m, 2H), 7.34-7.27 (m, 2H), 7.25-7.15 (m, 1H), 6.95-6.86 (m, 2H), 6.83-6.74 (m, 1H), 5.97-5.87 (m, 1H), 5.39-5.16 (m, 2H), 4.80 (s, 2H), 4.69-4.62 (m, 2H), 4.26-4.12 (m, 4H), 3.13-3.00 (m, 1H), 2.91-2.79 (m, 1H). LC-MS (ESI, m/z): [M+H]⁺ 502.

The following compounds were synthesized as outlined for the preparation of Example-3-6-07 employing appropriate starting materials in the table.

| | Structure and IUPAC name |
|---|---|
| | Fmoc-protected starting material-A |
| | Starting material-B |
| Example | NMR and MS |
| 3-6-08 | 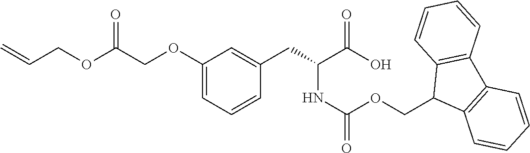 [SMILES: C=CCOC(=O)COc1cccc(C[C@@H](NC(=O)OCC2c3ccccc3-c3ccccc32)C(=O)O)c1] |
| | (2R)-2-({[(9H-fluoren-9-yl)methoxy]carbonyl}amino)-3-{3-[2-oxo-2- |
| | (prop-2-en-1-yloxy)ethoxy]phenyl}propanoic acid |
| | methyl (2S)-2-[[(9H-fluoren-9-ylmethoxy)carbonyl]amino]-3- |
| | iodopropanoate (CAS: 527696-77-1) |
| | Substantially identical to Example-3-6-07 |

### Example-3-7-01: (2S)-3-[3-(acetamidomethyl)phenyl]-2-({[(9H-fluoren-9-yl)methoxy]carbonyl}(methyl)amino)propanoic acid

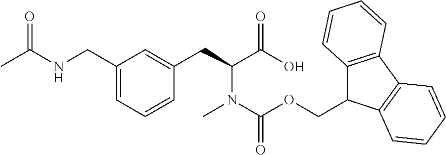 [SMILES: CC(=O)NCc1cccc(C[C@@H](C(=O)O)N(C)C(=O)OCC2c3ccccc3-c3ccccc32)c1]

To a suspension of Zn (0.50 g, 10.75 mmol) in DMF (4.3 mL) at rt were added 1,2-dibromoethane (0.019 mL, 0.22 mmol) and chlorotrimethylsilane (0.029 mL, 0.22 mmol). The mixture was stirred at 60° C. for 30 min. To the mixture at rt was added Reference example-3-1-01 (1.00 g, 2.15 mmol). The mixture was stirred at rt for 1.5 h. Then the mixture of Pd₂(dba)₃ CHCl₃ adduct (0.11 g, 0.11 mmol), and SPhos (0.18 g, 0.43 mmol) in DMF (2.1 mL) was stirred at 60° C. for 30 min. To the Pd complex mixture at rt were added N-(3-bromobenzyl)acetamide (0.588 g, 2.58 mmol) and organozinc reagent. The mixture was stirred at 50° C. for 3 h. The reaction was quenched with EtOAc and 2M aq. HCl at 0° C. The mixture was filtered. The filtrate was extracted with EtOAc. The organic extract was washed with brine, dried over Na₂SO₄, filtered, and then concentrated. The resulting residue was purified by silica gel flash column chromatography (gradient, Heptane/EtOAc=100/0 to 0/100). Appropriate fractions were concentrated. To the resulting residue (0.34 g, 0.69 mmol) in 2-Propanol (14 mL) at 0° C. were added CaCl₂ (0.92 g, 8.26 mmol), and a solution of lithium hydroxide (0.05 g, 2.07 mmol) in water (6 mL). The mixture was stirred at rt for 16 h, and then IPA was concentrated under reduced pressure. The mixture was diluted with EtOAc and water. The pH of the mixture was adjusted to pH 3 with 1M aq. HCl. The resulting mixture was extracted with EtOAc. The organic extract was washed with H₂O, dried over Na₂SO₄, filtered, and then concentrated. The resulting residue was purified by silica gel flash column chromatography (gradient, CH₂Cl₂/MeOH=100/0 to 60/40) to afford the title compound (0.26 g, 0.53 mmol). ¹H NMR (500 MHz, DMSO-d₆) δ 13.07-12.79 (m, 1H), 8.33-8.24 (m, 1H), 7.89 (d, J=7.5 Hz, 2H), 7.61-7.48 (m, 2H), 7.46-7.36 (m, 2H), 7.35-7.26 (m, 2H), 7.24-7.17 (m, 1H), 7.12 (s, 1H), 7.10-6.89 (m, 2H), 4.84-4.66 (m, 1H), 4.33-4.11 (m, 5H), 3.25-3.08 (m, 1H), 3.07-2.82 (m, 1H), 2.75-2.65 (m, 3H), 1.90-1.77 (m, 3H). LC-MS (ESI, m/z): [M+H]⁺ 473.

The following compounds were synthesized as outlined for the preparation of Example-3-7-01 employing appropriate starting materials in the table.

| | Structure and IUPAC name |
|---|---|
| | Fmoc-protected starting material-A |
| | Starting material-B |
| Example | NMR and MS |
| 3-7-02 | 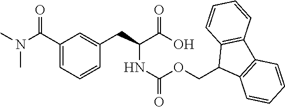 [SMILES: CN(C)C(=O)c1cccc(C[C@H](NC(=O)OCC2c3ccccc3-c3ccccc32)C(=O)O)c1] |
| | (2S)-3-[3-(dimethylcarbamoyl)phenyl]-2-({[(9H-fluoren-9- |
| | yl)methoxy]carbonyl}amino)propanoic acid |
| | methyl (2R)-2-({[(9H-fluoren-9-yl)methoxy]carbonyl}amino)- |
| | 3-iodopropanoate (CAS: 156017-42-4) |
| | 3-Bromo-N,N-dimethylbenzamide (CAS: 24167-51-9) |
| | ¹H NMR (500 MHZ, MeOD-d₄) δ 7.81 (d, J = 7.6 Hz 2H), |
| | 7.67-7.57 (m, 2H), 7.43-7.38 (m, 2H), 7.37-7.24 (m, 6H), |
| | 4.45-4.38 (m, 1H), 4.32-4.21 (m, 2H), 4.17-4.11 (m, 1H), |
| | 3.33-3.24 (m, 1H), 3.10-2.98 (m, 4H), 2.94-2.85 |
| | (m, 3H). LC-MS (ESI, m/z): [M + H]⁺ 459. |

### Example-3-7-03: (2S)-3-(3-carbamoylphenyl)-2-({[(9H-fluoren-9-yl)methoxy]carbonyl}(methyl)amino)propanoic acid

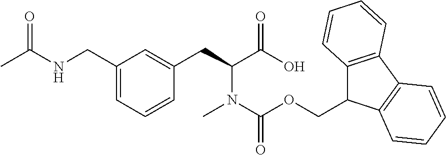 [SMILES: CC(=O)NCc1cccc(C[C@@H](C(=O)O)N(C)C(=O)OCC2c3ccccc3-c3ccccc32)c1]

To a solution of (2S)-3-(3-cyanophenyl)-2-({[(9H-fluoren-9-yl)methoxy]carbonyl}(methyl)amino)propanoic acid (CAS: 2642331-80-2, 16.00 g, 37.52 mmol) in 1,4-dioxane (312 mL) and water (312 mL) at rt were added (E)-acetaldehyde oxime (CAS: 5780-37-0, 4.44 g, 75.00 mmol), and copper(II) oxide (0.45 g, 5.63 mmol). The mixture was stirred at 100° C. for 16 h. 1,4-dioxane was removed under reduced pressure. The mixture was extracted with EtOAc. The resulting mixture was extracted with EtOAc. The organic extract was washed with H₂O and brine, dried over Na₂SO₄, filtered, and then concentrated. The resulting residue was purified by silica gel flash column chromatography (gradient, CH₂Cl₂/MeOH=100/0 to 85/15) to afford the title compound (11.85 g, 26.7 mmol). ¹H NMR (500 MHz, DMSO-d₆) δ 12.97 (br, 1H), 7.99-7.77 (m, 4H), 7.75-7.65 (m, 1H), 7.61-7.12 (m, 9H), 4.82 (br, 1H), 4.27-4.08 (m, 3H), 3.30-3.17 (m, 1H), 3.15-2.91 (m, 1H), 2.80-2.65 (m, 3H). LC-MS (ESI, m/z): [M+H]⁺ 445.

### Example-3-7-04: (2R)-3-(3-carbamoylphenyl)-2-({[(9H-fluoren-9-yl)methoxy]carbonyl}amino)propanoic acid

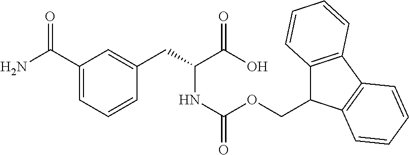 [SMILES: NC(=O)c1cccc(C[C@@H](NC(=O)OCC2c3ccccc3-c3ccccc32)C(=O)O)c1]

To a suspension of Zn (7.24 g, 111 mmol) in DMF (44.3 mL) at rt were added 1,2-dibromoethane (0.19 mL, 2.22 mmol) and chlorotrimethylsilane (0.29 mL, 2.22 mmol). The mixture was stirred at 60° C. for 30 min. To the mixture at rt was added methyl (2S)-2-({[(9H-fluoren-9-yl)methoxy]carbonyl}amino)-3-iodopropanoate (CAS: 527696-77-1, 10.00 g, 22.16 mmol). The mixture was stirred at rt for 1.5 h. Then the mixture of Pd₂(dba)₃ CHCl₃ adduct (1.15 g, 1.11 mmol), and SPhos (1.82 g, 4.43 mmol) in DMF (22.2 mL) was stirred at 60° C. for 30 min. To the Pd complex mixture at rt were added 3-bromobenzamide (CAS: 22726-00-7, 5.32 g, 26.6 mmol) and organozinc reagent. The mixture was stirred at 50° C. for 3 h. The reaction was quenched with EtOAc and 2M aq. HCl at 0° C. The mixture was filtered. The filtrate was extracted with EtOAc. The organic extract was washed with brine, dried over Na₂SO₄, filtered, and then concentrated. The resulting residue was purified by silica gel flash column chromatography (gradient, Heptane/EtOAc=100/0 to 0/100). Appropriate fractions were concentrated. To the resulting residue (3.4 g, 7.65 mmol) in 1,4-dioxane (25.50 mL) and water (12.75 mL) at rt were added I₂M HCl aq. (12.75 mL, 153 mmol). The mixture was stirred at 80° C. for 1 h, and then diluted with EtOAc and water. The mixture was extracted twice with EtOAc. The combined organic extracts were washed with H₂O and brine, dried over Na₂SO₄, filtered, and then concentrated. The resulting residue was purified by silica gel flash column chromatography (gradient, Heptane/EtOAc=95/5 to 60/40) to afford the title compound (1.46 g, 3.39 mmol). ¹H NMR (500 MHz, DMSO-d₆) δ 13.02-12.69 (m, 1H), 8.00-7.92 (m, 1H), 7.91-7.85 (d, 2H), 7.85-7.82 (s, 1H), 7.81-7.69 (m, 2H), 7.67-7.58 (m, 2H), 7.45-7.38 (m, 3H), 7.37-7.25 (m, 4H), 4.31-4.11 (m, 4H), 3.18-3.06 (m, 1H), 2.98-2.85 (m, 1H). LC-MS (ESI, m/z): [M+H]⁺ 431.
The following compounds were synthesized as outlined for the preparation of Example-3-7-04 employing appropriate starting materials in the table.

| | Structure and IUPAC name |
|---|---|
| | Fmoc-protected starting material-A |
| | Starting material-B |
| Example | NMR and MS |
| 3-7-05 | 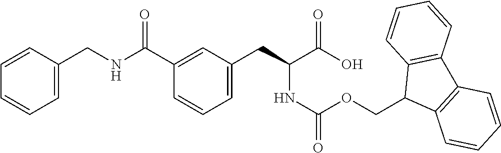 [SMILES: O=C(N[C@@H](Cc1cccc(C(=O)NCc2ccccc2)c1)C(=O)O)OCC1c2ccccc2-c2ccccc21] |
| | (2S)-3-[3-(benzylcarbamoy])phenyl]-2-({[(9H-fluoren-9- |
| | yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | 3-Iodo-N-(phenylmethyl)benzamide (CAS: 82082-51-7) |
| | ¹H NMR (500 MHz, DMSO-d₆) δ 13.49-12.37 (m, 1H), 9.10-8.91 (m, |
| | 1H), 7.91-7.81 (m, 3H), 7.79-7.69 (m, 1H), 7.67-7.58 (m, 2H), 7.52- |
| | 7.33 (m, 5H), 7.33-7.17 (m, 7H), 4.52-4.41 (m, 2H) 4.25-4.06 (m, 4H), |
| | 3.18-3.11 (m, 1H), 2.98-2.87 (m, 1H). LC-MS (ESI, m/z): [M + H]⁺ 535. |

### Example-3-7-06: (2S)-2-({[(9H-fluoren-9-yl)methoxy]carbonyl}(methyl)amino)-3-(3-{[(prop-2-en-1-yloxy)carbonyl]amino}phenyl)propanoic acid

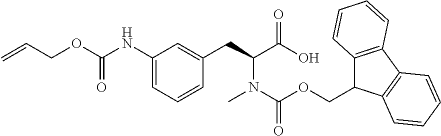 [SMILES: C=CCOC(=O)Nc1cccc(C[C@@H](C(=O)O)N(C)C(=O)OCC2c3ccccc3-c3ccccc32)c1]

To a solution of (2S)-2-[(tert-butoxycarbonyl)amino]-3-(3-nitrophenyl)propanoic acid (CAS: 131980-29-5, 10.00 g, 32.23 mmol) in THF (200 mL) at 0° C. was added NaH (2.55 g, 106.36 mmol). The mixture was stirred at 0° C. for 10 min. To the mixture at 0° C. was added Iodomethane (13.72 g, 96.68 mmol). The mixture was stirred at 0° C. for 1 h. Then the mixture was stirred at rt for 24 h. The reaction was quenched by aq. citric acid. The mixture was extracted three times with EtOAc. The combined organic extracts were washed twice with brine, dried over Na₂SO₄, filtered, and concentrated. The resulting residue was purified by silica gel flash column chromatography (gradient, CH₂Cl₂/MeOH=99/1 to 90/10). Appropriate fractions were concentrated. To the solution of obtained compound (10.00 g, 30.83 mmol) in MeOH (110 mL) at rt was added Pd/C (9.00 g, wet). The mixture was stirred at rt for 2 h under H₂ atmosphere, and then filtered. The filtrate was concentrated. To the obtained compound (9.00 g, 30.58 mmol) in 1,4-dioxane (45 mL) and water (45 mL) at rt were added DIEA (7.90 g, 61.15 mmol), NaHCO₃ (7.71 g, 91.73 mmol), and allyl chloroformate (4.42 g, 36.69 mmol). The mixture was stirred at rt for 16 h. The reaction was quenched by aq. citric acid. The mixture was extracted three times with EtOAc. The combined organic extracts were washed twice with brine, dried over Na₂SO₄, filtered, and concentrated. The resulting residue was purified by silica gel flash column chromatography (gradient, CH₂Cl₂/MeOH=99/1 to 90/10). Appropriate fractions were concentrated. To a solution of the resulting residue in CH₂Cl₂ (45.00 mL) at rt was added TFA (20.00 mL). The mixture was stirred at rt for 2 h, and then concentrated. To the obtained compound (4.70 g, 16.89 mmol) in 1,4-dioxane (60 mL) and water (60 mL) at rt were added NaHCO₃ (4.26 g, 50.66 mmol) and Fmoc-OSu (5.70 g, 16.89 mmol). The mixture was stirred at rt for 5 h. The reaction was quenched by aq. citric acid. The mixture was extracted three times with EtOAc. The combined organic extracts were washed twice with brine, dried over Na₂SO₄, filtered, and concentrated. The resulting residue was purified by reversed phase silica gel flash column chromatography (C18 silica gel, gradient, H₂O/CH₃CN with 0.1% formic acid=45/55 to 30/70) to afford the title compound (4.15 g, 8.37 mmol). ¹H NMR (500 MHz, DMSO-d₆) δ 12.93 (br, 1H), 9.79-9.48 (m, 1H), 7.88 (d, J=7.5 Hz, 2H), 7.60-7.36 (m, 5H), 7.34-7.24 (m, 3H), 7.21-7.13 (m, 1H), 6.89-6.71 (m, 1H), 5.98-5.87 (m, 1H), 5.36-5.27 (m, 1H), 5.24-5.15 (m, 1H), 4.82-4.70 (m, 1H), 4.61-4.50 (m, 2H), 4.26-4.10 (m, 3H), 3.21-3.10 (m, 1H), 3.06-2.85 (m, 1H), 2.80-2.68 (m, 3H). LC-MS (ESI, m/z): [M+H]⁺ 501.

### Example-3-8-01: (2S)-3-[4-(dimethylcarbamoyl)phenyl]-2-({[(9H-fluoren-9-yl)methoxy]carbonyl}amino)propanoic acid (CAS: 2350011-46-8)

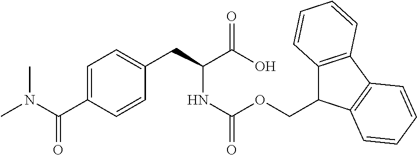 [SMILES: CN(C)C(=O)c1ccc(C[C@H](NC(=O)OCC2c3ccccc3-c3ccccc32)C(=O)O)cc1]

Example-3-8-01 was synthesized as outlined for the preparation of Example-3-2-06 employing appropriate starting materials methyl (R)-2-((((9H-fluoren-9-yl)methoxy)carbonyl)amino)-3-iodopropanoate (CAS: 156017-42-4) and 4-bromo-N,N-dimethylbenzamide (CAS: 18469-37-9). ¹H NMR (300 MHz, DMSO-d₆) δ 12.76 (s, 1H), 7.87 (d, J=7.5 Hz, 2H), 7.79-7.49 (m, 3H), 7.46-7.22 (m, 8H), 4.28-4.05 (m, 4H), 3.21-2.66 (m, 8H). LC-MS (ESI, m/z): [M+H]⁺ 459.

### Example-3-8-02: (2S)-3-(4-carbamoylphenyl)-2-(methylamino)propanoic acid

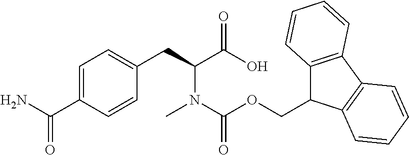 [SMILES: CN(C(=O)OCC1c2ccccc2-c2ccccc21)[C@@H](Cc1ccc(C(N)=O)cc1)C(=O)O]

To a solution of (2S)-3-(4-carbamoylphenyl)-2-({[(9H-fluoren-9-yl)methoxy]carbonyl}(methyl)amino)propanoic acid (CAS: 2642331-82-4, 10 g, 24 mmol) in 1,4-dioxane (200 mL) and H₂O (200 mL) were added acetaldoxime (CAS: 107-29-9, 2.77 g, 46.9 mmol) and CuO (CAS: 1317-38-0, 0.28 g, 3.5 mmol). The resulting mixture was stirred overnight at 100° C. The resulting mixture was concentrated under reduced pressure. The residue was purified by reverse phase silica gel flash column chromatography (C18 silica gel, gradient, H₂O/CH₃CN=95/5 to 0/100) to afford the title compound (5.3 g, 11 mmol). ¹H NMR (400 MHz, DMSO-d₆) δ 12.89 (br, 1H), 7.95-7.85 (m, 3H), 7.83-7.72 (m, 2H), 7.62-7.23 (m, 8H), 7.12 (d, J=8.0 Hz, 1H), 4.89-4.62 (m, 1H), 4.40-4.12 (m, 3H), 3.29-2.82 (m, 2H), 2.72-2.63 (m, 3H). LC-MS (ESI, m/z): [M+H]⁺ 445.

### Example-3-8-03: (2S)-2-({[(9H-fluoren-9-yl)methoxy]carbonyl}amino)-3-{4-[(2,5,8,11-tetraoxatridecan-13-yl)carbamoyl]phenyl}propanoic acid

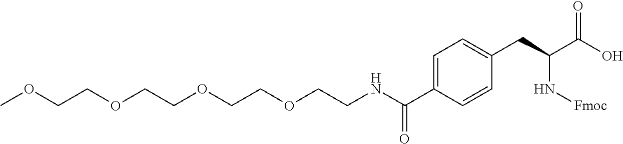

To a solution of 4-iodobenzoic acid (CAS: 619-58-9, 23.9 g, 96.5 mmol) in DMF (100 mL) were added HATU (44.0 g, 116 mmol) and DIPEA (24.9 g, 193 mmol). The resulting mixture was stirred for 5 min at room temperature. To the above mixture was added 2,5,8,11-tetraoxatridecan-13-amine (CAS: 85030-56-4, 20 g, 96 mmol). The mixture was stirred at rt for 3 h, and then diluted with H₂O. The mixture was extracted three times with EtOAc. The combined organic extracts were washed three times with brine, dried over Na₂SO₄, filtered, and concentrated. The resulting residue was purified by silica gel flash column chromatography (isocratic, EtOAc/petroleum ether=1/1). Appropriate fractions were collected and concentrated. To a suspension of Zn (15.3 g, 233 mmol) in DMF (350 mL) was added I₂ (11.8 g, 46.7 mmol). To the mixture at rt was added methyl (R)-2-((((9H-fluoren-9-yl)methoxy)carbonyl)amino)-3-iodopropanoate (CAS: 156017-42-4, 35.1 g, 77.8 mmol). The mixture was stirred at rt for 1 h. To the mixture were added obtained material in the last step (34 g, 77.8 mmol), Pd₂(dba)₃ (4.02 g, 3.89 mmol), and SPhos (6.38 g, 15.6 mmol). The mixture was stirred at 50° C. for 3 h. The mixture was quenched with H₂O and then filtered. The filtrate was extracted three times with EtOAc. The organic extracts were washed with brine, dried over Na₂SO₄, filtered, and then concentrated. The resulting residue was purified by silica gel flash column chromatography (isocratic, petroleum ether/EtOAc=30/70). Appropriate fractions were concentrated. To a solution of the obtained material (16 g, 25.2 mmol) in 2-propanol (180 mL) at 0° C. were added CaCl₂ (44.8 g, 403 mmol). To the mixture at 0° C. was added LiOH·H₂O (4.23 g, 101 mmol) in H₂O (60 mL). The mixture was stirred at rt 2 h. The pH of the mixture was adjusted to pH 4 with sartd. aq. NaH₂PO₄. The resulting mixture was extracted three times with EtOAc. The combined organic extracts were washed with brine, dried over Na₂SO₄, filtered, and then concentrated. The resulting residue was purified by silica gel flash column chromatography (isocratic, petroleum ether/EtOAc=50/50) to afford the title compound (12.0 g, 19.3 mmol). ¹H NMR (400 MHz, DMSO-d₆) δ 8.45 (t, J=5.6 Hz, 1H), 7.88 (d, J=7.5 Hz, 2H), 7.83-7.75 (m, 3H), 7.63 (t, J=7.9 Hz, 2H), 7.46-7.20 (m, 6H), 4.36-4.07 (m, 4H), 3.66-3.45 (m, 16H), 3.22 (s, 3H), 3.14 (dd, J=13.8, 4.4 Hz, 1H), 2.92 (dd, J=13.8, 10.6 Hz, 1H). LC-MS (ESI, m/z): [M+H]⁺ 621.

### Example-3-8-04: (2S)-4-(2-{[(tert-butoxy)carbonyl]amino}pyridin-3-yl)-2-({[(9H-fluoren-9-yl)methoxy]carbonyl}amino)butanoic acid

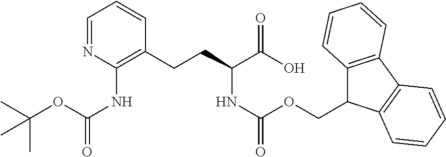 [SMILES: CC(C)(C)OC(=O)Nc1ncccc1CC[C@H](NC(=O)OCC1c2ccccc2-c2ccccc21)C(=O)O]

To a suspension of Zn (13.4 g, 205 mmol) in DMF (250 mL) was added I₂ (2.60 g, 10.2 mmol). The mixture was stirred at rt for 10 min. To the mixture at rt was added Reference example-3-1-03 (38.1 g, 81.8 mmol) and I₂ (2.60 g, 10.2 mmol). The mixture was stirred at rt for an additional 30 min. To the mixture were added 3-iodopyridin-2-amine (CAS: 104830-06-0, 15.0 g, 68.2 mmol), Pd₂(dba)₃ CHCl₃ adduct (1.25 g, 1.21 mmol), and SPhos (1.40 g, 3.41 mmol). The mixture was stirred at 60° C. for an additional 3 h, and then filtered. The filtrate was diluted with H₂O. The mixture was extracted three times with EtOAc. The combined organic extracts were washed with brine, dried over Na₂SO₄, filtered, and then concentrated. The resulting residue was purified by silica gel flash column chromatography (isocratic, petroleum ether/EtOAc=80/20). Appropriate fractions were concentrated. To the obtained material (10.0 g, 23.2 mmol) in t-BuOH (400 mL) at rt was added NaI (5.21 g, 34.8 mmol), Boc₂O (7.59 g, 34.8 mmol). The mixture was stirred at 40° C. for 1 day, and then concentrated. The resulting residue was purified by silica gel flash column chromatography (isocratic, petroleum ether/EtOAc=1/1). Appropriate fractions were concentrated. To a mixture of the obtained material (4 g, 7.5 mmol) in 2-propanol (400 mL) and H₂O (120 mL) at 0° C. were added CaCl₂ (13.4 g, 120 mmol) and LiOH·H₂O (1.26 g, 30.1 mmol). The mixture was stirred at rt for 3 h. The pH of the mixture was adjusted to pH 6 with aq. citric acid. The resulting mixture was extracted three times with EtOAc. The combined organic extracts were washed three times with brine, dried over Na₂SO₄, filtered, and then concentrated. The resulting residue was purified by reversed phase silica gel flash column chromatography (C18 silica gel, gradient, H₂O/CH₃CN=100/0 to 0/100) to afford the title compound (2.00 g, 3.87 mmol). ¹H NMR (400 MHz, DMSO-d₆) δ 12.78 (br, 1H), 9.13 (s, 1H), 8.24 (dd, J=4.8, 2.0 Hz, 1H), 7.94-7.87 (m, 2H), 7.77-7.70 (m, 2H), 7.70-7.52 (m, 2H), 7.46-7.38 (m, 2H), 7.38-7.25 (m, 2H), 7.19 (dd, J=7.6, 4.8 Hz, 1H), 4.39-4.14 (m, 3H), 3.97-3.83 (m, 1H), 2.75-2.55 (m, 2H), 2.10-1.97 (m, 1H), 1.90-1.77 (m, 1H), 1.42 (s, 9H). LC-MS (ESI, m/z): [M+H]⁺ 518.

### Example-3-8-05: ((2S)-6-(carbamoylamino)-2-({[(9H-fluoren-9-yl)methoxy]carbonyl}(methyl)amino)hexanoic acid

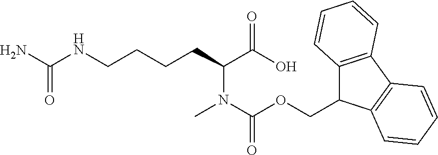 [SMILES: CN(C(=O)OCC1c2ccccc2-c2ccccc21)[C@@H](CCCCNC(N)=O)C(=O)O]

To a solution of (2S)-6-{[(tert-butoxy)carbonyl]amino}-2-({[(9H-fluoren-9-yl)methoxy]carbonyl}(methyl)amino)hexanoic acid (CAS: 197632-76-1, 20.0 g, 41.4 mmol) in DCM (120 ml) were added 4M HCl in CPME (120 mL) and stirred for 2 h. The reaction solution was concentrated in vacuo, and dried at 50° C. under vacuum. To the obtained material (3.00 g, 7.16 mmol) in THF (21 ml) and H₂O (14 ml) was added potassium cyanate (581 mg, 7.16 mmol) at 0° C. After stirring the mixture at 23° C. overnight, to the reaction was added potassium cyanate (581 mg, 7.16 mmol) at 0° C. The mixture was stirred at 23° C. overnight, and then diluted with EtOAc. The separated organic extracts was washed with water and brine, dried over Na₂SO₄, filtered, and then concentrated to afford title compound (3.05 g, 6.09 mmol). ¹H NMR (500 MHz, DMSO-d₆) δ 12.94-12.63 (m, 1H) 7.90 (t, J=7.5 Hz, 2H), 7.70-7.58 (m, 2H), 7.46-7.38 (m, 2H), 7.38-7.27 (m, 2H), 5.95-5.84 (1H), 5.44-5.28 (m, 2H), 4.53-4.21 (m, 4H), 2.99-2.86 (m, 2H), 2.77-2.68 (m, 3H), 1.88-0.99 (m, 6H). LC-MS (ESI, m/z): [M+H]⁺ 426.

### Example-3-9-01: (2S)-4-{4-[(tert-butoxy)carbonyl]phenyl}-2-({[(9H-fluoren-9-yl)methoxy]carbonyl}amino)butanoic acid

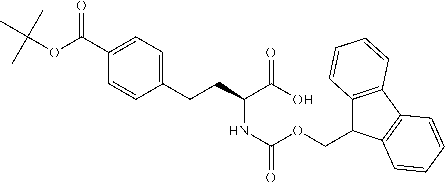 [SMILES: CC(C)(C)OC(=O)c1ccc(CC[C@H](NC(=O)OCC2c3ccccc3-c3ccccc32)C(=O)O)cc1]

To a suspension of Zn (11.40 g, 174.4 mmol) in DMF (300 mL) at rt was added I₂ (2.22 g, 8.75 mmol). The mixture was stirred at rt for 5 min. To the mixture at rt were added Reference example-3-1-03 (CAS: 1354921-71-3, 30.0 g, 64.5 mmol) and I₂ (2.22 g, 8.75 mmol). The mixture was stirred at rt for 30 min. To a mixture of tert-butyl 4-bromobenzoate (CAS: 59247-47-1, 15.0 g, 58.3 mmol), Pd₂(dba)₃ (1.60 g, 1.70 mmol), and SPhos (1.20 g, 2.92 mmol). The mixture was stirred at 60° C. for 3 h. The reaction mixture was cooled to 5-10° C. with a water/ice bath. The solids were filtered out. The resultant solution was diluted with EtOAc. The mixture was washed three times with brine, dried over Na₂SO₄, filtered, and then concentrated. The resulting residue was purified by silica gel flash column chromatography (gradient, petroleum ether/EtOAc=84/16). Appropriate fractions were concentrated. To a mixture of the obtained material (40.0 g, 77.6 mmol) in 2-propanol (900 mL) and H₂O (300 mL) at 0° C. were added CaCl₂ (138 g, 1.24 mol) and LiOH·H₂O (13.0 g, 310 mmol). The mixture was stirred at rt overnight. The pH of the mixture was adjusted to pH 7 with 5 M aq. NaH₂PO₄. The resulting mixture was extracted three times with EtOAc. The combined organic extracts was concentrated. The resulting residue was purified by silica gel flash column chromatography (gradient, petroleum ether/EtOAc=50/50). to afford the title compound (25.6 g, 51.2 mmol). ¹H NMR (300 MHz, DMSO-d₆) δ 12.66 (br, 1H), 7.94-7.72 (m, 6H), 7.47-7.28 (m, 6H), 4.41-4.14 (m, 3H), 3.96-3.84 (m, 1H), 2.84-2.61 (m, 2H), 2.10-1.85 (m, 2H), 1.54 (s, 9H). LC-MS (ESI, m/z): [M−H]⁻ 500.

### Example-3-9-02: (2S)-2-({[(9H-fluoren-9-yl)methoxy]carbonyl}(methyl)amino)-6-[(2H-1,2,3,4-tetrazol-5-yl)amino]hexanoic acid

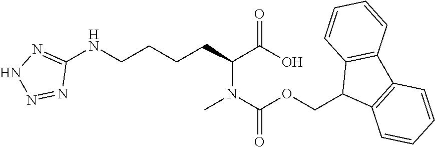 [SMILES: CN(C(=O)OCC1c2ccccc2-c2ccccc21)[C@@H](CCCCNc1nn[nH]n1)C(=O)O]

To a solution of (2S)-6-(tert-butoxy)-2-({[(9H-fluoren-9-yl)methoxy]carbonyl}amino)hexanoic acid (CAS: 1354752-71-8, 3.00 g, 7.05 mmol) in toluene (94.2 mL) at rt were added CSA (164 mg, 0.705 mmol) and paraformaldehyde (1.06 g, 35.3 mmol). The mixture was stirred at 80° C. for 3 h. The resulting residue was diluted with EtOAc and satd. aq NaHCO₃. The phases were separated, and the aqueous layer was extracted twice with EtOAc. The combined organic extracts were washed with brine, dried over Na₂SO₄, filtered, and then concentrated. The resulting residue was purified by silica gel flash column chromatography (gradient, hexane/EtOAc=100/0 to 50/50). Appropriate fractions were concentrated. To the resulting residue (3.08 g, 7.04 mmol) in 1,2-dichloroethane (35.2 mL) at 0° C. were added triethylsilane (11.2 mL, 70.4 mmol) and TFA (21.7 mL, 282 mmol). The mixture was stirred at rt overnight and then concentrated. To the resulting residue (2.54 g, 5.30 mmol) in MeCN (26.5 mL) at 0° C. were added DIPEA (2.78 mL, 15.9 mmol) and tert-butyl 2-(methylamino)acetate hydrochloride (CAS: 637-96-7, 962 mg, 5.30 mmol). The mixture was stirred at the same temperature for 3 h. The resultant solution was diluted with EtOAc, H₂O, and 1M aq HCl. The phases were separated, and the aqueous layer was extracted twice with EtOAc. The combined organic extracts were washed with brine, dried over Na₂SO₄, filtered, and then concentrated. The resulting residue was purified by silica gel flash column chromatography (gradient, DCM/MeOH=100/0 to 70/30). Appropriate fractions were concentrated. To the resulting residue (2.00 g, 5.22 mmol) in DCM (40.0 mL) was added Dess-Martin Periodinane (CAS: 87413-09-0, 2.43 g, 5.74 mmol). The mixture was stirred at room temperature for 30 min. The resultant solution was diluted with DCM and H₂O. The phases were separated, and the organic layer was washed with brine, dried over Na₂SO₄, filtered, and then concentrated. The resulting residue was purified by silica gel flash column chromatography (gradient, DCM/MeOH=100/0 to 90/10). Appropriate fractions were concentrated. To the resulting residue (2.00 g, 5.24 mmol) in MeOH (26.2 mL) were added 2H-1,2,3,4-tetrazol-5-amine (CAS: 4418-61-5, 0.892 g, 10.5 mmol) and AcOH (0.300 ml, 5.24 mmol). After the reaction mixture was stirred for 1 h at rt, sodium cyanotrihydroborate (CAS: 25895-60-7, 0.494 g, 7.87 mmol) was added to the reaction mixture. The mixture was stirred at the same temperature for 1 h. The resultant solution was diluted with EtOAc and H₂O. The phases were separated, and the aqueous layer was extracted twice with EtOAc. The combined organic extracts were washed with brine, dried over Na₂SO₄, filtered, and then concentrated. The resulting residue was purified by silica gel flash column chromatography (gradient, DCM/MeOH=100/0 to 50/50). Appropriate fractions were concentrated. To the resulting residue (2.36 g, 5.24 mmol) in DCM (26.2 mL) were added 1H-imidazole (CAS: 288-32-4, 1.43 g, 21.0 mmol) and tert-butylchlorodimethylsilane (CAS: 18162-48-6, 1.58 mg, 10.5 mmol). The mixture was stirred at the same temperature for 1 h. The resultant solution was diluted with EtOAc and H₂O. The phases were separated, and the aqueous layer was extracted twice with EtOAc. The combined organic extracts were washed with brine, dried over Na₂SO₄, filtered, and then concentrated. The resulting residue was purified by silica gel flash column chromatography (gradient, DCM/MeOH=100/0 to 70/30) to afford the title compound (388 mg, 0.732 mmol). ¹H NMR (500 MHz, DMSO-d₆) δ 7.94-7.85 (m, 2H), 7.70-7.57 (m, 2H), 7.45-7.36 (m, 2H), 7.36-7.26 (m, 2H), 7.19-7.06 (m, 1H), 4.52-4.20 (m, 4H), 3.18-3.09 (m, 2H), 2.75-2.70 (m, 3H), 1.87-1.43 (m, 4H), 1.27-1.12 (m, 2H). LC-MS (ESI, m/z): [M+H]⁺ 451.

### Example-3-9-03: (2S)-6-{[(tert-butoxy)carbonyl](1H-1,2,3,4-tetrazol-5-yl)amino}-2-({[(9H-fluoren-9-yl)methoxy]carbonyl}(methyl)amino)hexanoic acid

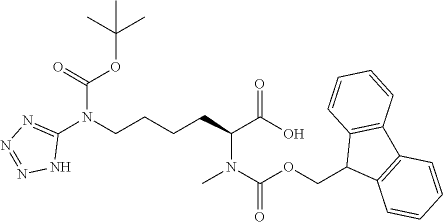 [SMILES: CN(C(=O)OCC1c2ccccc2-c2ccccc21)[C@@H](CCCCN(C(=O)OC(C)(C)C)c1nnn[nH]1)C(=O)O]

To a solution of Example-3-9-02 (250 mg, 0.555 mmol) in THF (2.78 mL) at rt were added Boc₂O (291 mg, 1.33 mmol) and DIPEA (0.233 mL, 1.33 mmol). The mixture was stirred at 50° C. for 1 h. The resulting residue was diluted with EtOAc and H₂O. The phases were separated, and the aqueous layer was extracted twice with EtOAc. The combined organic extracts were washed with brine, dried over Na₂SO₄, filtered, and then concentrated. The resulting residue was purified by silica gel flash column chromatography (gradient, DCM/MeOHc=100/0 to 50/50) to afford the title compound (185 mg, 0.336 mmol). ¹H NMR (500 MHz, DMSO-d₆) δ 12.94 (br, 1H), 10.38 (m, 1H), 7.96-7.81 (m, 2H), 7.70-7.58 (m, 2H), 7.48-7.24 (m, 4H), 4.50-4.07 (m, 6H), 2.74-2.66 (s, 3H), 1.99-1.00 (m, 15H). LC-MS (ESI, m/z): [M+H]⁺ 551.

### Example-3-9-04: (2S)-6-(2-acetamidoacetamido)-2-({[(9H-fluoren-9-yl)methoxy]carbonyl}(methyl)amino)hexanoic acid

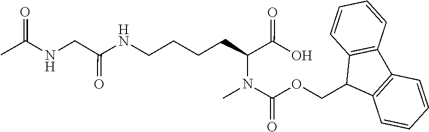 [SMILES: CC(=O)NCC(=O)NCCCC[C@@H](C(=O)O)N(C)C(=O)OCC1c2ccccc2-c2ccccc21]

To a solution of prop-2-en-1-yl (2S)-6-amino-2-({[(9H-fluoren-9-yl)methoxy]carbonyl}(methyl)amino)hexanoate hydrochloride (U.S. 63/310,905, 3.00 g, 6.54 mmol) and acetylglycine (CAS: 543-24-8, 0.918 g, 7.84 mmol) in DMF (32.7 mL) at 0° C. were added DIPEA (1.27 g, 9.80 mmol) and HATU (2.98 g, 7.84 mmol). The mixture was stirred at rt for 3 h, and then diluted with H₂O. The mixture was extracted three times with EtOAc. The combined organic extracts were washed with brine, dried over Na₂SO₄, filtered, and concentrated. The resulting residue was purified by silica gel flash column chromatography (gradient, hexane/EtOAc=90/10 to 0/100, then DCM/MeOH 100/0 to 80/20). Appropriate fractions were collected and concentrated. To a solution of the product (3.26 g, 6.25 mmol) in CH₂Cl₂ (125 mL) were added phenylsilane (1.54 ml, 12.5 mmol) and Pd(PPh₃)₄ (0.361 g, 0.312 mmol). The mixture was stirred at room temperature for 2 h, and then concentrated. The resulting residue was purified by silica gel flash column chromatography (gradient, CH₂Cl₂/MeOH=100/0 to 70/30). Appropriate fractions were collected and concentrated to afford the title compound (2.03 g, 4.17 mmol). ¹H NMR (500 MHz, DMSO-d₆) δ 12.80 (br, 1H), 8.11-8.01 (m, 1H), 7.92-7.88 (m, 2H), 7.82 (br, 1H), 7.68-7.60 (m, 2H), 7.45-7.39 (m, 2H), 7.38-7.29 (m, 2H), 4.52-4.23 (m, 4H), 3.63 (d, J=5.3 Hz, 2H), 3.10-2.98 (m, 2H), 2.76-2.70 (m, 3H), 1.90-1.52 (m, 5H), 1.47-1.28 (m, 2H), 1.21-1.00 (m, 2H). LC-MS (ESI, m/z): [M+H]⁺ 482.

The following compounds were synthesized as outlined for the preparation of Example-3-9-04 employing appropriate starting materials in the table.

| | Structure and IUPAC name |
|---|---|
| | Starting material acid-A |
| | Fmoc-protected starting material-B |
| Example | MS data |
| 3-9-05 | 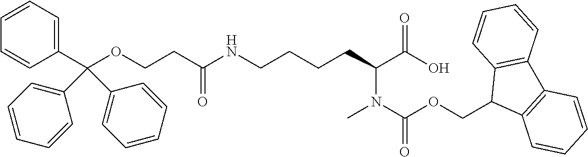 [SMILES: CN(C(=O)OCC1c2ccccc2-c2ccccc21)[C@@H](CCCCNC(=O)CCOC(c1ccccc1)(c1ccccc1)c1ccccc1)C(=O)O] |
| | (2S)-2-({[(9H-fluoren-9-yl)methoxy]carbonyl}(methyl)amino)-6-[3- |
| | (triphenylmethoxy)propanamido]hexanoic acid |
| | 3-(triphenylmethoxy)propanoic acid (CAS: 24243-01-4) |
| | prop-2-en-1-yl (2S)-6-amino-2-({[(9H-fluoren-9- |
| | yl)methoxy]carbonyl}(methyl)amino)hexanoate hydrochloride |
| | (US63/310905) |
| | ¹H NMR (500 MHz, DMSO-d₆) δ 12.82 (m, 1H), 7.97 (t, J = 5.3 Hz, 1H), |
| | 7.92-7.86 (m, 2H), 7.67-7.53 (m, 2H), 7.44-7.17 (m, 19H), 4.50-4.18 |
| | (m, 4H), 3.19-3.01 (m, 4H), 2.73-2.68 (m, 3H), 2.42-2.29 (m, 2H), 1.87- |
| | 1.53 (m, 2H), 1.51-1.33 (m, 2H), 1.31-1.07 (m, 2H). LC-MS (ESI, |
| | m/z): not found. |

### Example-3-9-06: (2S)-6-[4-(tert-butoxy)-4-oxobutanamido]-2-({[(9H-fluoren-9-yl)methoxy]carbonyl}(methyl)amino)hexanoic acid

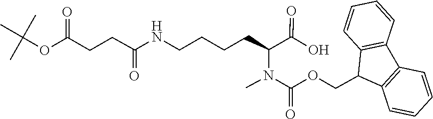 [SMILES: CN(C(=O)OCC1c2ccccc2-c2ccccc21)[C@@H](CCCCNC(=O)CCC(=O)OC(C)(C)C)C(=O)O]

To a solution of prop-2-en-1-yl (2S)-6-amino-2-({[(9H-fluoren-9-yl)methoxy]carbonyl}(methyl)amino)hexanoate hydrochloride (U.S. 63/310,905, 3.40 g, 5.19 mmol) and 4-(tert-butoxy)-4-oxobutanoic acid (CAS: 15026-17-2, 0.994 g, 5.70 mmol) in DMF (25.9 mL) at 0° C. were added DIPEA (1.01 g, 7.78 mmol) and HATU (2.366 g, 6.22 mmol). The mixture was stirred at rt for 3 h, and then diluted with H₂O. The mixture was extracted with EtOAc. The combined organic extracts were washed with brine, dried over Na₂SO₄, filtered, and concentrated. The resulting residue was purified by silica gel flash column chromatography (gradient, hexane/EtOAc=100/0 to 0/100). Appropriate fractions were collected and concentrated. To a solution of the product (2.80 g, 4.84 mmol) in CH₂Cl₂ (46 mL) at 0° C. were added phenylsilane (1.19 mL, 9.68 mmol) and Pd(PPh₃)₄ (0.140 g, 0.121 mmol). The mixture was stirred at 0° C. for 1 h, and then concentrated. The resulting residue was purified by silica gel flash column chromatography (gradient, CH₂Cl₂/MeOH=100/0 to 70/30). Appropriate fractions were collected and concentrated to afford the title compound (2.66 g, 4.74 mmol). ¹H NMR (500 MHz, DMSO-d₆) δ 12.81 (br, 1H), 7.93-7.87 (m, 2H), 7.84-0.78 (m, 1H), 7.69-7.59 (m, 2H), 7.47-7.38 (m, 2H), 7.38-7.27 (m, 2H), 4.51-4.23 (m, 4H), 3.06-2.94 (m, 2H), 2.77-2.70 (m, 3H), 2.42-2.34 (m, 2H), 2.29-2.22 (m, 2H), 1.86-1.52 (m, 2H), 1.42-1.32 (s, 9H), 1.32-1.00 (m, 4H). LC-MS (ESI, m/z): [M+H]⁺ 539.

### Example-3-9-07: (2S)-6-(cyclopropylformamido)-2-({[(9H-fluoren-9-yl)methoxy]carbonyl}(methyl)amino)hexanoic acid

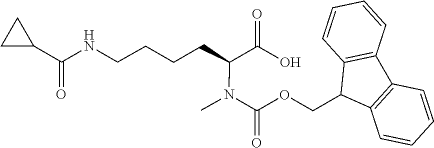 [SMILES: CN(C(=O)OCC1c2ccccc2-c2ccccc21)[C@@H](CCCCNC(=O)C1CC1)C(=O)O]

To a solution of cyclopropanecarboxylic acid (CAS: 1759-53-1, 0.646 g, 7.50 mmol) and 1-hydroxybenzotriazole monohydrate (CAS: 80029-43-2, 1.72 g, 11.3 mmol) in DMF (7.5 mL) at 0° C. was added 1-(3-dimethylaminopropyl)-3-ethylcarbodiimide hydrochloride (CAS: 25952-53-8, 1.73 g, 9.00 mmol). The mixture was stirred at rt for 10 min, a solution of prop-2-en-1-yl (2S)-6-amino-2-({[(9H-fluoren-9-yl)methoxy]carbonyl}(methyl)amino)hexanoate hydrochloride (U.S. 63/310,905, 3.44 g, 7.50 mmol) in DMF (11.6 mL) and Et₃N (1.25 mL, 9.00 mmol) were added at 0° C. The mixture was stirred at 40° C. for 1.5 h and concentrated. The mixture was dissolved in EtOAc, washed with 2M aq. Na₂HPO₄, satd. aq. NaHCO₃, water, brine, dried over Na₂SO₄, filtered, and concentrated. The resulting residue was purified by silica gel flash column chromatography (gradient, hexane/EtOAc=100/0 to 90/10). Appropriate fractions were collected and concentrated. To a solution of the product (2.15 g, 4.39 mmol) in CH₂C12 (21.9 mL) at 0° C. were added phenylsilane (1.08 mL, 8.78 mmol) and Pd(PPh₃)₄ (0.127 g, 0.110 mmol). The mixture was stirred at 0° C. for 1.5 h. The reaction mixture was extracted twice with 1M aq. NaHCO₃. The combined aqueous layers were washed with DCM, acidified to pH3 with 2M aq. NaH₂PO₄ at 0° C., and extracted twice with EtOAc. The combined organic extracts were washed with brine, dried over Na₂SO₄, filtered, and concentrated to afford the title compound (1.23 g, 2.71 mmol). ¹H NMR (500 MHz, DMSO-d₆) δ 12.83 (br, 1H), 8.01 (t, J=5.4 Hz, 1H), 7.92-7.87 (m, 2H), 7.69-7.60 (m, 2H), 7.45-7.38 (m, 2H), 7.37-7.28 (m, 2H), 4.53-4.22 (m, 4H), 3.10-2.96 (m, 2H), 2.75-2.70 (m, 3H), 1.86-1.04 (m, 7H), 0.66-0.56 (m, 4H). LC-MS (ESI, m/z): [M+H]⁺ 451.

### Example-3-9-08: (2S)-2-({[(9H-fluoren-9-yl)methoxy]carbonyl}(methyl)amino)-6-propanamidohexanoic acid

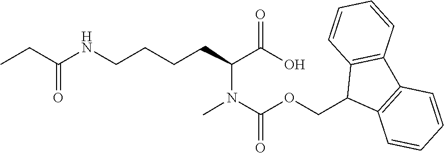 [SMILES: CCC(=O)NCCCC[C@@H](C(=O)O)N(C)C(=O)OCC1c2ccccc2-c2ccccc21]

To a solution of prop-2-en-1-yl (2S)-6-amino-2-({[(9H-fluoren-9-yl)methoxy]carbonyl}(methyl)amino)hexanoate hydrochloride (U.S. 63/310,905, 1.00 g, 2.39 mmol) in THF (7.96 mL) at rt was added N,O-bis(trimethylsilyl)acetamide (CAS: 10416-59-8, 2.92 mL, 11.94 mmol). The mixture was stirred at rt for 30 min, propionyl chloride (CAS: 79-03-8, 0.250 mL, 2.86 mmol) was added at 0° C. The mixture was stirred at 0° C. for 1 h, then at rt for 2 h and diluted with EtOAc and satd. aq. NaHCO₃. The phases were separated, and the organic layer was extracted with satd. aq. NaHCO₃. The combined aqueous layers were acidified with 1M aq. HCl at 0° C., and extracted with EtOAc. The combined organic extracts were washed with brine, dried over Na₂SO₄, filtered, and concentrated to afford the title compound (0.762 g, 1.64 mmol). ¹H NMR (500 MHz, DMSO-d₆) δ 12.79 (br, 1H), 7.94-7.86 (m, 2H), 7.76-7.68 (m, 1H), 7.68-7.60 (m, 2H), 7.46-7.37 (m, 2H), 7.37-7.28 (m, 2H), 4.52-4.22 (m, 4H), 3.05-2.97 (m, 2H), 2.75-2.70 (m, 3H), 2.09-1.98 (m, 2H), 1.85-1.54 (m, 2H), 1.44-1.22 (m, 2H), 1.21-1.03 (m, 2H), 0.97 (t, J=7.6 Hz, 3H). LC-MS (ESI, m/z): [M+H]⁺ 439.

### Example-3-10-01: (2S)-2-({[(9H-fluoren-9-yl)methoxy]carbonyl}(methyl)amino)-6-(3-methoxypropanamido)hexanoic acid

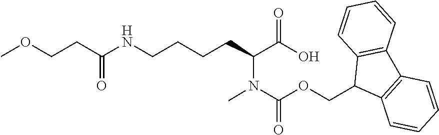 [SMILES: COCCC(=O)NCCCC[C@@H](C(=O)O)N(C)C(=O)OCC1c2ccccc2-c2ccccc21]

To a solution of Prop-2-en-1-yl (2S)-6-amino-2-({[(9H-fluoren-9-yl)methoxy]carbonyl}(methyl)amino)hexanoate hydrochloride (U.S. 63/310,905, 3.00 g, 4.58 mmol), 3-Methoxypropionic acid (CAS: 2544-06-1, 0.524 g, 5.03 mmol) and DIPEA (1.18 g, 9.15 mmol) in DMF (22.9 mL) was added HATU (2.09 g, 5.49 mmol). The mixture was stirred at rt for 3 h, and then diluted with H₂O. The mixture was extracted with EtOAc. The combined organic extracts were dried over Na₂SO₄, filtered, and concentrated. The resulting residue was purified by silica gel flash column chromatography (gradient, hexane/EtOAc=100/0 to 0/100). Appropriate fractions were collected and concentrated. To a solution of the product (2.10 g, 4.13 mmol) in CH₂C12 (46.0 mL) were added phenylsilane (0.894 g, 8.26 mmol) and Pd(PPh₃)₄ (0.119 g, 0.103 mmol) at 0° C. The mixture was stirred at 0° C. for 1 h, and then concentrated. The resulting residue was purified by silica gel flash column chromatography (gradient, CH₂Cl₂/MeOH=100/0 to 70/30). Appropriate fractions were collected and concentrated to furnish the title compound (1.66 g, 3.37 mmol). ¹H NMR (500 MHz, DMSO-d₆) δ 12.81 (br, 1H), 7.98-7.86 (m, 2H), 7.86-7.75 (m, 1H), 7.69-7.59 (m, 2H), 7.47-7.38 (m, 2H), 7.38-7.25 (m, 2H), 4.53-4.20 (m, 4H), 3.54-3.44 (m, 2H), 3.54-3.44 (m, 3H), 3.24-3.09 (m, 2H), 2.78-2.66 (m, 3H), 1.87-1.49 (m, 2H), 1.49-0.94 (m, 4H). LC-MS (ESI, m/z): [M+H]⁺ 469.

The following compounds were synthesized as outlined for the preparation of Example-3-10-01 employing appropriate starting materials in the table.

| | Structure and IUPAC name |
|---|---|
| | Starting material |
| Example | NMR and MS |
| 3-10-02 | 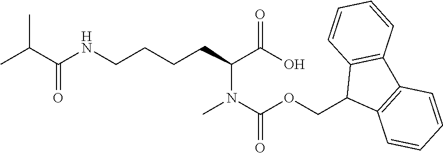 [SMILES: CC(C)C(=O)NCCCC[C@@H](C(=O)O)N(C)C(=O)OCC1c2ccccc2-c2ccccc21] |
| | (2S)-2-({[(9H-fluoren-9-yl)methoxy]carbonyl}(methyl)amino)-6-(2- |
| | methylpropanamido)hexanoic acid |
| | 2-methylpropanoic acid (CAS: 79-31-2) |
| | ¹H NMR (500 MHz, DMSO-d₆) δ 12.84 (br, 1H), 7.94-7.86 (m, 2H), 7.73- |
| | 7.59 (m, 3H), 7.47-7.34 (m, 2H), 7.34-7.26 (m, 2H), 4.54-4.20 (m, |
| | 4H), 3.08-2.93 (m, 2H), 2.82-2.67 (m, 3H), 2.35-2.24 (m, 1H), 1.88- |
| | 1.48 (m, 2H), 1.48-1.23 (m, 2H), 1.23-1.01 (m, 2H), 1.01-0.91 (m, 6H). |
| | LC-MS (ESI, m/z): [M + H]⁺ 453. |
| 3-10-03 | 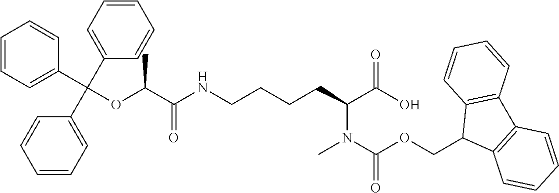 [SMILES: C[C@H](OC(c1ccccc1)(c1ccccc1)c1ccccc1)C(=O)NCCCC[C@@H](C(=O)O)N(C)C(=O)OCC1c2ccccc2-c2ccccc21] |
| | (2S)-2-({[(9H-fluoren-9-yl)methoxy]carbonyl}(methyl)amino)-6-[(2S)-2- |
| | (triphenylmethoxy)propanamido]hexanoic acid |
| | (2S)-2-(triphenylmethoxy)propanoic acid (CAS: 173152-70-0) |
| | ¹H NMR (500 MHZ, DMSO-d₆) δ 12.94-12.68 (m, 1H), 7.94-7.85 (m, |
| | 2H), 7.67-7.58 (m, 2H), 7.45-7.18 (m, 20H), 5.51-4.17 (m, 4H), 3.86 |
| | (dd, J = 6.5, 13.0 Hz, 1H), 2.87-2.75 (m, 2H), 2.75-2.67 (m, 3H), 1.85- |
| | 1.48 (m, 2H), 1.36-1.20 (m, 2H), 1.13-0.94 (m, 2H), 0.94-0.83 (m, 3H). |
| | LC-MS (ESI, m/z): [M + H]⁺ not found. |
| 3-10-04 | 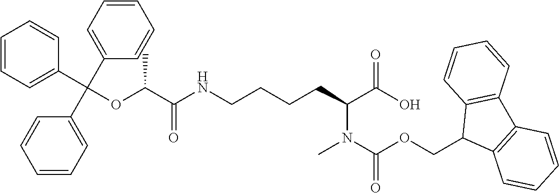 [SMILES: C[C@@H](OC(c1ccccc1)(c1ccccc1)c1ccccc1)C(=O)NCCCC[C@@H](C(=O)O)N(C)C(=O)OCC1c2ccccc2-c2ccccc21] |
| | (2S)-2-({[(9H-fluoren-9-yl)methoxy]carbonyl}(methyl)amino)-6-[(2R)-2- |
| | (triphenylmethoxy)propanamido]hexanoic acid |
| | (2R)-2-(triphenylmethoxy)propanoic acid (CAS: 2511741-49-2) |
| | ¹H NMR (500 MHz, DMSO-d₆) δ 12.92-12.70 (m, 1H), 7.94-7.84 (m, |
| | 2H), 7.66-7.57 (m, 2H), 7.45-7.19 (m, 20H), 4.51-4.19 (m, 4H), 3.86 |
| | (dd, J = 6.5, 13.0 Hz, 1H), 2.86-2.75 (m, 2H), 2.75-2.67 (m, 3H), 1.84- |
| | 1.45 (m, 2H), 1.20-1.14 (m, 2H), 0.94-0.87 (m, 3H). LC-MS (ESI, m/z): |
| | [M − Trt + H]⁺ 455. |
| 3-10-05 | 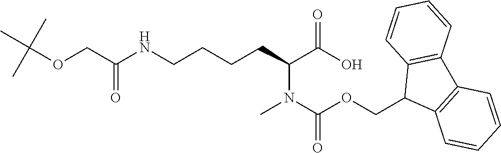 [SMILES: CN(C(=O)OCC1c2ccccc2-c2ccccc21)[C@@H](CCCCNC(=O)COC(C)(C)C)C(=O)O] |
| | (2S)-6-[2-(tert-butoxy)acetamido]-2-({[(9H-fluoren-9- |
| | yl)methoxy]carbonyl}(methyl)amino)hexanoic acid |
| | 2-(tert-butoxy)acetic acid (CAS: 13211-32-0) |
| | ¹H NMR (500 MHz, DMSO-d₆) δ 12.81 (br, 1H), 7.96-7.83 (m, 2H), 7.71- |
| | 7.15 (m, 7H), 4.54-4.19 (m, 4H), 3.72 (s, 2H), 3.17-3.01 (m, 2H), 2.81- |
| | 2.67 (m, 3H), 1.89-1.53 (m, 2H), 1.53-1.32 (m, 2H), 1.32-1.11 (m, |
| | 11H). LC-MS (ESI, m/z): [M + H]⁺ 497. |
| 3-10-06 | 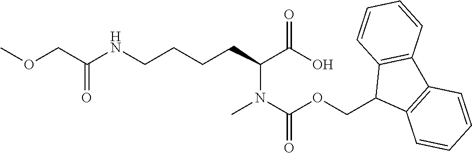 [SMILES: COCC(=O)NCCCC[C@@H](C(=O)O)N(C)C(=O)OCC1c2ccccc2-c2ccccc21] |
| | (2S)-2-({[(9H-fluoren-9-yl)methoxy]carbonyl}(methyl)amino)-6-(2- |
| | methoxyacetamido)hexanoic acid |
| | 2-methoxyacetic acid (CAS: 625-45-6) |
| | ¹H NMR (500 MHZ, DMSO-d₆) δ 12.83 (br, 1H), 7.96-7.85 (m, 2H), 7.83- |
| | 7.73 (m, 1H), 7.69-7.61 (m, 2H), 7.46-7.38 (m, 2H), 7.38-7.27 (m, |
| | 2H), 4.51-4.20 (m, 4H), 3.79-3.72 (m, 2H), 3.30-3.24 (m, 3H), 3.12- |
| | 3.00 (m, 2H), 2.77-2.67 (m, 3H), 1.88-1.50 (m, 2H), 1.50-1.21 (m, 2H), |
| | 1.21-0.98 (m, 2H). LC-MS (ESI, m/z): [M + H]⁺ 455. |

### Example-3-10-07: (2S)-2-({[(9H-fluoren-9-yl)methoxy]carbonyl}(methyl)amino)-6-(phenylformamido)hexanoic acid

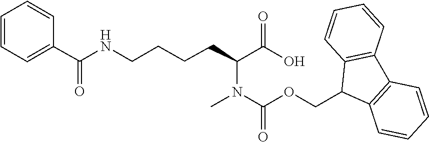 [SMILES: CN(C(=O)OCC1c2ccccc2-c2ccccc21)[C@@H](CCCCNC(=O)c1ccccc1)C(=O)O]

To a solution of (2S)-6-{[(tert-butoxy)carbonyl]amino}-2-({[(9H-fluoren-9-yl)methoxy]carbonyl}(methyl)amino)hexanoic acid (CAS: 197632-76-1, 20.0 g, 41.4 mmol) in CH₂Cl₂ (118 mL) was added 4M HCl in CPME (118 mL). The mixture was stirred for 2 h, and then concentrated. To the resulting residue (1.00 g, 2.39 mmol) in THF (7.96 mL) at rt was added BSA (2.43 g, 11.9 mmol). The mixture was stirred at rt for 0.5 h, and then cooled to 0°. To the mixture was added benzoyl chloride (CAS: 98-88-4, 0.336 g, 2.39 mmol) at 0° C. The mixture was stirred at rt for 2 h, and then diluted with aq. NaHCO₃. The mixture was extracted with EtOAc. The combined organic extracts were washed with aq. HCl, dried over Na₂SO₄, filtered, and concentrated to afford the title compound (0.915 g, 1.86 mmol). ¹H NMR (500 MHz, DMSO-d₆) δ 12.83 (br, 1H), 8.46 (s, 1H), 7.93-7.78 (m, 4H), 7.78-7.70 (m, 2H), 7.70-7.25 (m, 7H), 4.58-4.21 (m, 4H), 3.30-3.17 (m, 2H), 2.78-2.66 (m, 3H), 1.92-1.41 (m, 4H), 1.29-1.04 (m, 2H). LC-MS (ESI, m/z): [M+H]⁺ 487.

### Example-3-10-08: (2S)-2-({[(9H-fluoren-9-yl)methoxy]carbonyl}(methyl)amino)-6-(morpholin-4-yl)hexanoic acid

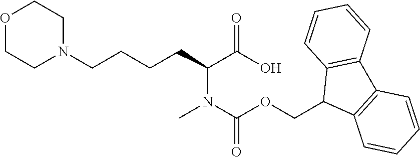 [SMILES: CN(C(=O)OCC1c2ccccc2-c2ccccc21)[C@@H](CCCCN1CCOCC1)C(=O)O]

To a solution of (2S)-2-({[(9H-fluoren-9-yl)methoxy]carbonyl}amino)-6-(morpholin-4-yl)hexanoic acid (CAS: 2349553-17-7, 2.23 g, 5.08 mmol) in acetic acid (25.4 ml) at rt were added CSA (0.118 g, 0.508 mmol) and paraformaldehyde (0.915 g, 30.5 mmol). The mixture was stirred at 80° C. for overnight, and then concentrated. The resulting residue was diluted with EtOAc and satd. aq NaHCO₃. The phases were separated, and the aqueous layer was extracted twice with EtOAc. The combined organic extracts were washed with brine, dried over MgSO₄, filtered, and then concentrated. The resulting residue was purified by silica gel flash column chromatography (gradient, CH₂Cl₂/MeOH=100/0 to 95/5). Appropriate fractions were concentrated. To the resulting residue (1.55 g, 3.44 mmol) and activated molecular sieves 4A in ClCH₂CH₂Cl (17.2 mL) at rt were added triethylsilane (6.59 ml, 19.7 mmol) and 2,2,2-trifluoroacetic acid (17.2 mL). The mixture was stirred at 50° C. for 4 h, filtered, washed with MeCN, concentrated, azeotroped with toluene, washed with IPE, and dissolved with aq. NaHCO₃. The aqueous layer was washed washed with DCM, acidified with aq. HCl, and extracted with CH₂Cl₂. The combined organic extracts were washed with brine, dried over MgSO₄, filtered, and then concentrated to afford the title compound (1.10 g, 3.44 mmol). ¹H NMR (500 MHz, DMSO-d₆) δ 7.95-7.86 (m, 2H), 7.69-7.58 (m, 2H), 7.47-7.29 (m, 4H), 4.58-4.18 (m, 3H), 3.89-3.56 (m, 4H), 3.46-3.17 (m, 3H), 2.96-2.60 (m, 7H), 1.89-1.40 (m, 4H), 1.25-0.99 (m, 2H). LC-MS (ESI, m/z): [M+H]⁺ 453.

### Example-3-11-01: (2S)-2-({[(9H-fluoren-9-yl)methoxy]carbonyl}(methyl)amino)-6-methanesulfonamidohexanoic acid

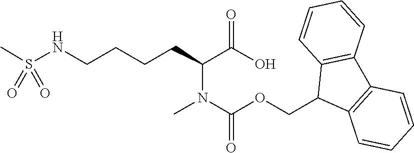 [SMILES: CN(C(=O)OCC1c2ccccc2-c2ccccc21)[C@@H](CCCCNS(C)(=O)=O)C(=O)O]

To a solution of (2S)-6-{[(tert-butoxy)carbonyl]amino}-2-({[(9H-fluoren-9-yl)methoxy]carbonyl}(methyl)amino)hexanoic acid (CAS: 197632-76-1, 20.0 g, 41.4 mmol) in CH₂Cl₂ (118 ml) at rt was added a 4M solution of HCl in CPME (118 ml, 472 mmol). The mixture was stirred at rt for 2 h, and then the resulting mixture was concentrated to obtain the material (18.0 g, 41.5 mmol). To a solution of the obtained compound (1.00 g, 2.39 mmol) in THF (7.96 ml) at rt was added BSA (2.92 ml, 11.9 mmol). The mixture was stirred at rt for 0.5 h, and then to the mixture at 0° C. was added methanesulfonyl chloride (CAS: 124-63-0, 0.223 ml, 2.86 mmol). The resulting mixture was stirred at 0° C. for 1 h and rt for 2 h, and then diluted with EtOAc and sat. aq. NaHCO₃. The phases were separated, and the organic layer was extracted with sat. aq. NaHCO₃, and then pH of the combined aqueous extracts was adjusted to 3 to 4 with 1M aq. HCl at 0° C. The mixture was extracted with EtOAc, and the organic extracts were dried over Na₂SO₄, filtered, and concentrated to afford the title compound (766 mg, 1.61 mmol). ¹H NMR (500 MHz, DMSO-d₆) δ 12.8 (br, 1H), 7.93-7.87 (dd, J=7.1, 7.1 Hz, 2H), 7.69-7.60 (m, 2H), 7.45-7.38 (m, 2H), 7.38-7.29 (m, 2H), 6.96 (s, 1H), 4.53-4.23 (m, 4H), 2.95-2.84 (m, 5H), 2.77-2.69 (m, 3H), 1.87-1.34 (m, 4H), 1.31-1.04 (m, 2H). LC-MS (ESI, m/z): [M+H]⁺ 489.

### Example-3-11-02: (2S)-2-({[(9H-fluoren-9-yl)methoxy]carbonyl}(methyl)amino)-6-(2-oxoimidazolidin-1-yl)hexanoic acid

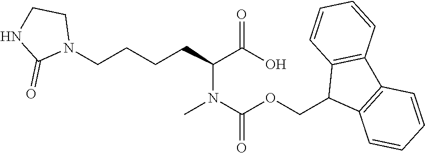 [SMILES: CN(C(=O)OCC1c2ccccc2-c2ccccc21)[C@@H](CCCCN1CCNC1=O)C(=O)O]

To a solution of prop-2-en-1-yl (2S)-6-amino-2-({[(9H-fluoren-9-yl)methoxy]carbonyl}(methyl)amino)hexanoate hydrochloride (U.S. 63/310,905, 6.07 g, 13.2 mmol) in CH₂Cl₂ (55.1 ml) at 0° C. were added 4A molecular sieves (CAS: 70955-01-0, 4.54 g) and tert-butyl N-(2-oxoethyl)carbamate (CAS: 89711-08-0, 1.76 g, 11.0 mmol). The mixture was stirred at 0° C. for 1 h, and then to the mixture at 0° C. was added sodium bis(acetyloxy)boranuidyl acetate (CAS: 56553-60-7, 3.04 g, 14.3 mmol). The mixture was stirred at 0° C. for 0.5 h and at rt for 1 h, and then to the mixture at 0° C. was added a 4M solution of HCl in MTHP (55.1 ml, 220 mmol). The mixture was stirred at rt for 1 h, and then the resulting mixture was filtered, and concentrated. The resulting residue was dissolved in acetone, filtered, and concentrated. To the resulting residue (7.12 g, 13.2 mmol) in THF (88.0 ml) at 0° C. were added DIPEA (4.85 ml, 27.8 mmol) and a solution of 1-(1H-imidazole-1-carbonyl)-1H-imidazole (CAS: 530-62-1, 2.15 g, 13.2 mmol). The mixture was stirred at 0° C. for 1 h and rt for 2 h, and then quenched with 2M aq. NaH₂PO₄ at 0° C. The resulting mixture was concentrated, and extracted twice with EtOAc. The combined organic extracts were washed with 1M aq. HCl, sat. aq. NaHCO₃ and brine, dried over Na₂SO₄, filtered, and then concentrated. The resulting residue was purified by silica gel flash column chromatography (gradient, hexane/EtOAc=50/50 to 0/100; then CH₂Cl₂/MeOH=100/0 to 80/20). Appropriate fractions were concentrated. To a solution of the resulting residue (1.35 g, 2.76 mmol) in CH₂Cl₂ (13.8 ml) at 0° C. were added phenylsilane (CAS: 694-53-1, 0.679 ml, 5.51 mmol) and Pd(PPh₃)₄ (80.0 mg, 69.2 □mol). The mixture was stirred at 0° C. for 1 h, and then extracted twice with 1M aq. NaHCO₃. The pH of the combined aqueous extracts was adjusted to 1 with 2M aq. HCl at 0° C., and then the mixture was extracted twice with EtOAc. The combined organic extracts were washed with water and brine, dried over Na₂SO₄, filtered, and then concentrated. The resulting residue was purified by silica gel flash column chromatography (gradient, CH₂Cl₂/MeOH=100/0 to 80/20) to afford the title compound (837 mg, 2.76 mmol). ¹H NMR (500 MHz, DMSO-d₆) δ 12.8 (br, 1H), 7.90 (dd, J=7.8, 7.8 Hz, 2H), 7.67-7.60 (m, 2H), 7.45-7.38 (m, 2H), 7.37-7.28 (m, 2H), 4.52-4.22 (m, 4H), 3.29-3.14 (m, 4H), 3.07-2.91 (m, 2H), 2.77-2.67 (m, 3H), 1.87-1.55 (m, 2H), 1.52-1.29 (m, 2H), 1.19-0.97 (m, 2H). LC-MS (ESI, m/z): [M+H]⁺ 452.

### Example-3-11-03: (2S)-2-({[(9H-fluoren-9-yl)methoxy]carbonyl}(methyl)amino)-6-(2-oxopyrrolidin-1-yl)hexanoic acid

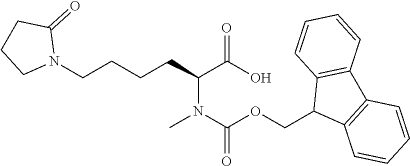 [SMILES: CN(C(=O)OCC1c2ccccc2-c2ccccc21)[C@@H](CCCCN1CCCC1=O)C(=O)O]

To a solution of prop-2-en-1-yl (2S)-6-amino-2-({[(9H-fluoren-9-yl)methoxy]carbonyl}(methyl)amino)hexanoate hydrochloride (U.S. 63/310,905, 30.0 g, 65.4 mmol) in CH₂Cl₂ (400 ml) at rt were added methyl 4-oxobutanoate (6.10 g, 52.5 mmol) and AcOH (11.2 ml, 195.5 mmol). The mixture was stirred for 1 h at rt, and then to the mixture was added sodium bis(acetyloxy)boranuidyl acetate (CAS: 56553-60-7, 16.6 g, 78.3 mmol). The resulting mixture was extracted three times with CH₂Cl₂. The combined organic extracts were washed with water, dried over Na₂SO₄, filtered, and then concentrated. To the resulting residue were added toluene (400 ml) and AcOH (200 ml). The mixture was stirred at 80° C. for overnight, and then purified by silica gel flash column chromatography (petroleum ether/EtOAc=50/50). Appropriate fractions were concentrated. To a solution of the resulting residue (17.7 g, 37.1 mmol) in CH₂Cl₂ (200 ml) were added Pd(PPh₃)₄ (4.29 g, 3.71 mmol) and phenylsilane (12.1 g, 111 mmol). The mixture was stirred for at rt 2 h, and then the pH of the mixture was adjusted to 7 with 5 M aq. NaH₂PO₄. The resulting mixture was extracted four times with EtOAc, and the combined organic extracts were purified by reversed phase silica gel flash column chromatography (C18 silica gel, gradient, H₂O/CH₃CN=100/0 to 0/100) to afford the title compound (11.2 g, 24.8 mmol). ¹H NMR (300 MHz, DMSO-d₆) δ 12.8 (br, 1H), 7.95-7.85 (m, 2H), 7.70-7.58 (m, 2H), 7.48-7.27 (m, 4H), 4.56-4.20 (m, 4H), 3.32-3.00 (m, 4H), 2.77-2.65 (m, 3H), 2.23-2.10 (m, 2H), 1.95-1.28 (m, 6H), 1.20-0.93 (m, 2H). LC-MS (ESI, m/z): [M+H]⁺ 451.

### Example-3-11-04: (2S)-2-({[(9H-fluoren-9-yl)methoxy]carbonyl}(methyl)amino)-6-(2-oxo-1,3-diazinan-1-yl)hexanoic acid

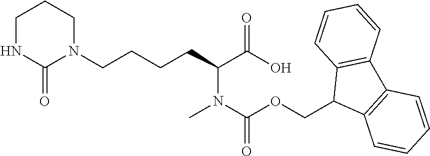 [SMILES: CN(C(=O)OCC1c2ccccc2-c2ccccc21)[C@@H](CCCCN1CCCNC1=O)C(=O)O]

To a solution of (2S)-6-{[(tert-butoxy)carbonyl]amino}-2-({[(9H-fluoren-9-yl)methoxy]carbonyl}(methyl)amino)hexanoic acid (CAS: 197632-76-1, 5.00 g, 10.4 mmol) in MeOH (8.39 ml, 207 mmol) at 0° C. was added sulfurooyl dichloride (CAS: 7719-09-7, 1.49 ml, 20.7 mmol). The mixture was stirred at rt for 2 h, then concentrated, and azeotroped three times with toluene. The resulting residue was triturated with MTBE, and then collected by filtration to obtain the material (3.18 g, 7.35 mmol). To a solution of the obtained material (2.35 g, 5.42 mmol) in THF (15.0 ml) at rt were added AcOH (0.776 ml, 13.6 mmol) and tert-butyl N-(3-oxopropyl)carbamate (783 mg, 4.52 mmol). The mixture was stirred at rt for 1 h, and then to the mixture at 0° C. was added sodium bis(acetyloxy)boranuidyl acetate (CAS: 56553-60-7, 1.15 g, 5.42 mmol). The mixture was stirred at rt for 1 h, and then quenched with 1M aq. HCl at 0° C. The resulting mixture was extracted twice with EtOAc. The combined organic extracts were dried over Na₂SO₄, filtered, and concentrated. The obtained residue was dissolved in MeOH (22.6 mL), and to the solution at 0° C. were added Boc₂O (1.48 g, 6.78 mmol) and Et₃N (2.52 ml, 18.1 mmol). The mixture was stirred at rt for 14 h, and then quenched with 2M aq. NaH₂PO₄ at 0° C. The mixture was concentrated, and extracted twice with EtOAc. The combined organic extracts were washed with brine, dried over Na₂SO₄, filtered, and concentrated. The resulting residue was purified by silica gel flash column chromatography (gradient, hexane/EtOAc=100/0 to 20/80). Appropriate fractions were concentrated. To the resulting residue (1.17 g, 1.79 mmol) in CH₂Cl₂ (8.97 ml) at 0° C. was added a 4M solution of HCl in MTHP (8.97 ml, 35.9 mmol). The mixture was stirred at rt for 4 h, and then the resulting mixture was concentrated. The resulting residue was triturated with hexane/MTBE (1/1). The resulting residue was collected by filtration, which was dissolved in THF (30.6 ml). To the solution at 0° C. were added 1-(1H-imidazole-1-carbonyl)-1H-imidazole (CAS: 530-62-1, 0.248 g, 1.53 mmol) and DIPEA (0.524 ml, 3.36 mmol). The mixture was stirred at 0° C. for 2 h, and then to the mixture at 0° C. was added DIPEA (0.524 ml, 3.36 mmol). The mixture was stirred at 0° C. for 1.5 h, rt for 19 h, and 40° C. for 2.5 h, and then quenched with 2M aq. NaH₂PO₄ at 0° C. The resulting mixture was concentrated, and extracted twice with EtOAc. The combined organic extracts were washed with brine, dried over Na₂SO₄, filtered, and concentrated. The resulting residue was purified by silica gel flash column chromatography (gradient, CH₂Cl₂/MeOH=100/0 to 80/20). Appropriate fractions were concentrated. To a solution of the obtained material (479 mg, 0.999 mmol) in 2-propanol (14.0 mL) at 0° C. were added CaCl₂ (CAS: 10043-52-4, 1.77 g, 16.0 mmol) and a solution of LiOH (CAS: 1310-65-2, 95.8 mg, 4.00 mmol) in H₂O (5.99 ml). The mixture was stirred at rt for 22 h, and then concentrated. The pH of the mixture was adjusted to pH 4 with 2M aq. NaH₂PO₄. The resulting mixture was extracted three times with EtOAc. The combined organic extracts were washed three times with brine, dried over Na₂SO₄, filtered, and then concentrated. The resulting residue was purified by silica gel flash column chromatography (gradient, CH₂Cl₂/MeOH=100/0 to 80/20) to afford the title compound (340 mg, 0.730 mmol). ¹H NMR (500 MHz, DMSO-d₆) δ 12.8 (br, 1H), 7.90 (dd, 7.3, 7.3 Hz, 2H), 7.68-7.59 (m, 2H), 7.45-7.39 (m, 2H), 7.37-7.28 (m, 2H), 4.51-4.22 (m, 4H), 3.23-3.02 (m, 6H), 2.78-2.68 (m, 3H), 1.86-1.65 (m, 4H), 1.64-1.29 (m, 2H), 1.17-0.96 (m, 2H). LC-MS (ESI, m/z): [M+H]⁺ 466.

### Example-3-11-05: (2S)-3-{4′-cyano-[1,1′-biphenyl]-3-yl}-2-({[(9H-fluoren-9-yl)methoxy]carbonyl}amino)propanoic acid

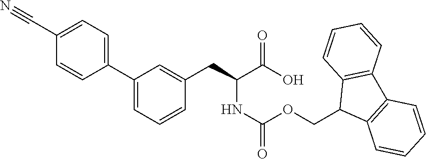 [SMILES: N#Cc1ccc(-c2cccc(C[C@H](NC(=O)OCC3c4ccccc4-c4ccccc43)C(=O)O)c2)cc1]

To a solution of (2S)-3-(3-bromophenyl)-2-({[(9H-fluoren-9-yl)methoxy]carbonyl}amino)propanoic acid (CAS: 220497-48-3, 15.0 g, 32.2 mmol) in THF (200 ml) were added (4-cyanophenyl)boronic acid (CAS: 126747-14-6, 7.09 g, 48.3 mmol), palladium(2+) diacetate (CAS: 3375-31-3, 1.44 g, 6.41 mmol), K₃PO₄ (20.5 g, 96.5 mmol), and 1,1′-Bis(di-tert-butylphosphino)ferrocene (CAS: 84680-95-5, 3.05 g, 6.46 mmol). The mixture was stirred at 50° C. for overnight, and then filtered. The pH of the filtrate was adjusted to 7 with aq. citric acid, and then the resulting mixture was extracted three times with EtOAc. The combined organic extracts were washed three times with brine, dried over Na₂SO₄, filtered, and concentrated. The resulting residue was purified by silica gel flash column chromatography (petroleum ether/EtOAc=1/2) to afford the title compound (7.10 g, 14.5 mmol). ¹H NMR (400 MHz, DMSO-d₆) δ 13.7 (br, 1H), 7.98-7.76 (m, 6H), 7.72-7.52 (m, 4H), 7.50-7.31 (m, 5H), 7.30-7.16 (m, 2H), 4.32-4.01 (m, 4H), 3.33-2.82 (m, 2H). LC-MS (ESI, m/z): [M−H]⁻ 487.

### Example-3-11-06: (2S)-2-({[(9H-fluoren-9-yl)methoxy]carbonyl}(methyl)amino)-6-(1H-1,2,3-triazol-1-yl)hexanoic acid

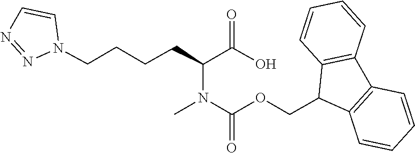 [SMILES: CN(C(=O)OCC1c2ccccc2-c2ccccc21)[C@@H](CCCCn1ccnn1)C(=O)O]

A solution of prop-2-en-1-yl (2S)-6-amino-2-({[(9H-fluoren-9-yl)methoxy]carbonyl}(methyl)amino)hexanoate hydrochloride (U.S. 63/310,905, 4.20 g, 9.15 mmol) in EtOAc was washed with sat. aq. NaHCO₃. The organic extract was dried over Na₂SO₄, filtered, and then concentrated. A mixture of oxaldehyde (CAS: 107-22-2, 1.61 ml, 14.2 mmol), 4-methylbenzene-1-sulfonohydrazide (CAS: 1576-35-8, 2.64 g, 14.2 mmol), AcOH (50.0 □l, 0.874 mmol), and MeOH (15.8 ml) was stirred at rt for 1 h, and then to the mixture was added the residue prepared above (2.00 g, 4.73 mmol). The mixture was stirred for 3 h, and then quenched with water. The resulting mixture was extracted with EtOAc, and the organic extract was dried over Na₂SO₄, filtered, and then concentrated. The resulting residue was purified by silica gel flash column chromatography (gradient, hexane/EtOAc=100/0 to 0/100). Appropriate fractions were concentrated. To the resulting residue (560 mg, 1.18 mmol) in CH₂Cl₂ (46.0 ml) were added Pd(PPh₃)₄ (34.1 mg, 29.5 □mol) and phenylsilane (0.291 ml, 2.36 mmol). The mixture was stirred at rt for 1 h, and then concentrated. The resulting residue was purified by silica gel flash column chromatography (gradient, CH₂Cl₂/MeOH=100/0 to 80/20) to afford the title compound (418 mg, 0.914 mmol). ¹H NMR (500 MHz, DMSO-d₆) δ 12.9-12.7 (m, 1H), 8.11 (s, 1H), 7.92-7.85 (m, 2H), 7.74-7.68 (m, 1H), 7.67-7.26 (m, 8H), 7.72-7.65 (m, 3H), 4.51-4.21 (m, 6H), 2.74-2.65 (m, 3H), 1.92-1.55 (m, 4H), 1.19-0.99 (m, 2H). LC-MS (ESI, m/z): [M+H]⁺ 435.

### Example-3-11-07: (2S)-5-acetamido-2-({[(9H-fluoren-9-yl)methoxy]carbonyl}(methyl)amino)pentanoic acid

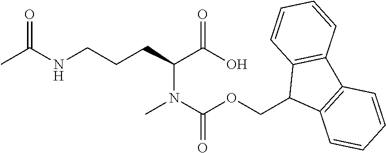 [SMILES: CC(=O)NCCC[C@@H](C(=O)O)N(C)C(=O)OCC1c2ccccc2-c2ccccc21]

To a suspension of (2S)-5-acetamido-2-({[(9H-fluoren-9-yl)methoxy]carbonyl}amino)pentanoic acid (CAS: 172690-48-1, 10.0 g, 25.2 mmol) in toluene (63.1 mL) at rt were added CSA (0.352 g, 1.51 mmol) and paraformaldehyde (CAS: 30525-89-4, 3.79 g, 126 mmol). The mixture was stirred at 80° C. for 5 h, and then diluted with EtOAc. The resulting mixture was washed with water and brine, dried over Na₂SO₄, filtered, and then concentrated. To a solution of the resulting residue (9.90 g, 24.2 mmol) in ClCH₂CH₂Cl (60.6 mL) at 0° C. were added 4A molecular sieves (CAS: 70955-01-0, 12.12 g), tris(propan-2-yl)silane (CAS: 6485-79-6, 49.7 ml, 242 mmol), and TFA (60.6 ml). The mixture was stirred at 40° C. for overnight, and then filtered. The filtrate was concentrated, and azeotroped three times with toluene. The resulting residue was purified by silica gel flash column chromatography (gradient, CH₂Cl₂/MeOH=100/0 to 95/5) to afford the title compound (2.71 g, 6.60 mmol). ¹H NMR (500 MHz, DMSO-d₆) δ 12.8 (br, 1H), 8.09-7.55 (m, 5H), 7.52-7.14 (m, 4H), 4.59-3.89 (m, 4H), 3.36-3.14 (m, 1H), 3.10-2.98 (m, 1H), 2.95-2.68 (m, 3H), 2.02-1.18 (m, 7H). LC-MS (ESI, m/z): [M+H]⁺ 435.

### Example-3-12-01: (2S)-2-({[(9H-fluoren-9-yl)methoxy]carbonyl}amino)-5-methanesulfonamidopentanoic acid

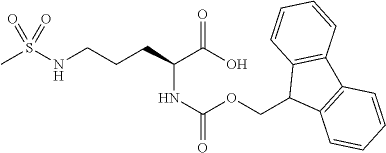 [SMILES: CS(=O)(=O)NCCC[C@H](NC(=O)OCC1c2ccccc2-c2ccccc21)C(=O)O]

To a solution of (2S)-5-amino-2-({[(9H-fluoren-9-yl)methoxy]carbonyl}amino)pentanoic acid hydrochloride (CAS: 201046-57-3, 7.00 g, 17.9 mmol) in THF (4.26 mL) at rt was added BSA (18.22 g, 90.0 mmol). The mixture at rt was stirred for 0.5 h. To the mixture was added methanesulfonyl chloride (2.67 g, 23.3 mmol) at 0° C. The reaction mixture was stirred at 0° C. for 1 h and rt for 3 h. The resulting mixture was diluted with EtOAc and satd. aq NaHCO₃. The phases were separated, and the organic layer was extracted with satd. aq NaHCO₃. The pH of the combined aqueous layers was adjusted to 3 to 4 with 1M aq. HCl at 0° C. The aqueous layer was extracted with EtOAc. The organic extract was dried over Na₂SO₄, filtered, and then concentrated. The resulting residue was purified by silica gel flash column chromatography (gradient, CH₂Cl₂/MeOH=100/0 to 96/4) to afford the title compound (6.35 g, 14.68 mmol). ¹H NMR (500 MHz, DMSO-d₆) δ 12.62 (br, 1H), 7.90 (d, J=7.5 Hz, 2H), 7.76-7.70 (m, 2H), 7.70-7.62 (m, 1H), 7.42 (t, J=7.5 Hz, 2H), 7.34 (t, J=7.5 Hz, 2H), 7.01-6.94 (m, 1H), 4.32-4.20 (m, 3H), 3.98-3.89 (m, 1H), 2.93 (q, J=6.7 Hz, 2H), 2.87 (s, 3H), 1.81-1.72 (m, 1H), 1.65-1.47 (m, 3H). LC-MS (ESI, m/z): [M+H]⁺ 433.

### Example-3-12-02: (2S)-2-({[(9H-fluoren-9-yl)methoxy]carbonyl}amino)-4-{[(pyridin-4-yl)methyl]carbamoyl}butanoic acid

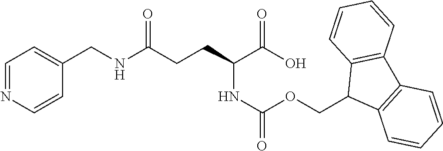 [SMILES: O=C(CC[C@H](NC(=O)OCC1c2ccccc2-c2ccccc21)C(=O)O)NCc1ccncc1]

To a solution of (4S)-5-(tert-butoxy)-4-({[(9H-fluoren-9-yl)methoxy]carbonyl}amino)-5-oxopentanoic acid (CAS: 84793-07-7, 30.00 g, 70.51 mmol) in DMF (300 mL) at 0° C. were added HATU (10.25 g, 26.97 mmol), DIPEA (5.81 g, 44.95 mmol) and 1-(pyridin-4-yl)methanamine (CAS: 3731-53-1, 7.63 g, 70.51 mmol). The mixture was stirred at rt for 5 min, and then diluted with H₂O. The mixture was extracted three times with EtOAc. The combined organic extracts were washed three times with brine, dried over Na₂SO₄, filtered, and concentrated. The resulting residue was purified by silica gel flash column chromatography (isocratic, CH₂Cl₂/MeOH=10/1). Appropriate fractions were collected and concentrated (20.0 g, 38.78 mmol). To a solution of the obtained material in 4M HCl in EtOAc (210 mL) was stirred at rt for 8 h. The solids were purified by re-crystallization from EtOAc to afford the title compound (10.03 g, 21.83 mmol). ¹H NMR (300 MHz, DMSO-d₆) δ 8.90-8.75 (m, 3H), 7.97-7.82 (m, 4H), 7.80-7.69 (m, 3H), 7.47-7.39 (m, 2H), 7.39-7.32 (m, 2H), 4.65-4.43 (m, 2H), 4.38-4.17 (m, 3H), 4.08-3.93 (m, 1H), 2.47-2.28 (m, 2H), 2.11-1.98 (m, 1H), 1.95-1.77 (m, 1H), 1.185 (s, 1H). LC-MS (ESI, m/z): [M+H]⁺ 460.

### Example-3-12-03: (2S)-2-({[(9H-fluoren-9-yl)methoxy]carbonyl}amino)-4-{[(pyridin-4-yl)methyl]carbamoyl}butanoic acid hydrochloride

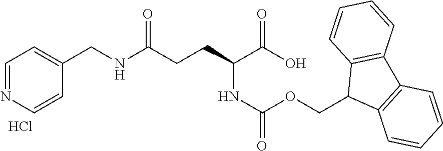 [SMILES: O=C(CC[C@H](NC(=O)OCC1c2ccccc2-c2ccccc21)C(=O)O)NCc1ccncc1 ; Cl]

A solution of (2S)-2-({[(9H-fluoren-9-yl)methoxy]carbonyl}amino)-4-{[(pyridin-4-yl)methyl]carbamoyl}butanoic acid (16.0 g, 34.8 mmol) in 4M HCl in CPME (174 mL) was stirred at rt for 3 h. The reaction mixture was diluted with diisopropyl ether. The precipitate was filtered, rinsed with diisopropyl ether and then dried under reduced pressure to afford the title compound (15.33 g, 29.0 mmol). ¹H NMR (500 MHz, DMSO-d₆) δ 12.62 (br, 1H), 8.84-8.78 (m, 2H), 8.77-8.72 (m, 1H), 7.90 (d, J=7.5 Hz, 2H), 7.86-7.82 (m, 2H), 7.77-7.70 (m, 3H), 7.46-7.39 (m, 2H), 7.34 (t, J=7.4 Hz, 2H), 4.59-4.45 (m, 2H), 4.35-4.19 (m, 3H), 4.07-3.90 (m, 1H), 3.147 (s, 1H), 2.43-2.29 (m, 2H), 2.11-1.99 (m, 1H), 1.89-1.79 (m, 1H). LC-MS (ESI, m/z): [M+H]⁺ 460.

### Example-3-12-04: (2S)-2-({[(9H-fluoren-9-yl)methoxy]carbonyl}amino)-4-{[(1s,3s)-3-{[(tert-butoxy)carbonyl]amino}cyclobutyl]carbamoyl}butanoicacid

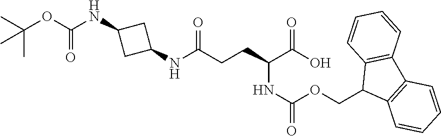 [SMILES: CC(C)(C)OC(=O)N[C@H]1C[C@@H](NC(=O)CC[C@H](NC(=O)OCC2c3ccccc3-c3ccccc32)C(=O)O)C1]

To a solution of (4S)-4-({[(9H-fluoren-9-yl)methoxy]carbonyl}amino)-5-oxo-5-(prop-2-en-1-yloxy)pentanoic acid (CAS: 144120-54-7, 12.00 g, 29.31 mmol) in DMF (120 mL) at rt were added HATU (16.72 g, 43.96 mmol), DIPEA (11.36 g, 87.93 mmol) and tert-butyl N-[(1s,3s)-3-aminocyclobutyl]carbamate (CAS: 1212395-34-0, 5.46 g, 29.31 mmol). The mixture was stirred at rt for 0.5 h, and then diluted with H₂O. The mixture was extracted three times with EtOAc. The combined organic extracts were washed with brine, dried over Na₂SO₄, filtered, and concentrated. The resulting residue was purified by silica gel flash column chromatography (gradient, EtOAc/petroleum ether=1/10 to 1/1). Appropriate fractions were concentrated (14.00 g, 24.24 mmol). To a solution of the obtained material in THF (140 mL) at rt was added Pd(PPh₃)₄ (2.80 g, 2.423 mmol). To the mixture at 0° C. was added morpholine (CAS: 110-91-8, 4.22 g, 48.470 mmol). The resulting mixture was stirred for 0.5 h at rt. The pH value of the solution was adjusted to 5 with 3 M aq. HCl. The resulting mixture was extracted three times with EtOAc. The combined organic extracts were washed with brine, dried over Na₂SO₄, filtered, and concentrated. The resulting residue was purified by reversed phase silica gel flash column chromatography (C18 silica gel, gradient, H₂O with 0.1% TFA/CH₃CN=50/50 to 20/80) to afford the title compound (5.015 g, 9.328 mmol). ¹H NMR (300 MHz, DMSO-d₆) δ 12.61 (br, 1H), 8.08-7.95 (m, 1H), 7.89 (d, J=7.5 Hz, 2H), 7.73 (d, J=7.4 Hz, 2H), 7.64 (d, J=7.9 Hz, 1H), 7.42 (t, J=7.4, 7.4 Hz, 2H), 7.37-7.30 (m, 2H), 7.17-7.06 (m, 1H), 4.35-4.14 (m, 3H), 3.99-3.85 (m, 1H), 3.85-3.71 (m, 1H), 3.65-3.50 (m, 1H), 3.45-3.22 (m, 1H), 2.47-2.34 (m, 2H), 2.23-2.10 (m, 2H), 1.84-1.65 (m, 3H), 1.36 (s, 9H). LC-MS (ESI, m/z): [M+H]⁺ 538.

### Example-3-12-05: (2S)-4-[(carbamoylmethyl)carbamoyl]-2-({[(9H-fluoren-9-yl)methoxy]carbonyl}(methyl)amino)butanoic acid

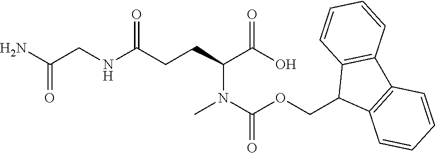 [SMILES: CN(C(=O)OCC1c2ccccc2-c2ccccc21)[C@@H](CCC(=O)NCC(N)=O)C(=O)O]

To a suspension of (4S)-5-(tert-butoxy)-4-({[(9H-fluoren-9-yl)methoxy]carbonyl}(methyl)amino)-5-oxopentanoicacid (CAS: 2444356-83-4, 2.5 g, 5.69 mmol) and 1H-1,2,3-benzotriazol-1-ol hydrate (CAS: 80029-43-2, 1.307 g, 8.53 mmol) in CH₂Cl₂ (5.69 ml) at 0° C. was added ({[3-(dimethylamino)propyl]imino}methylidene)(ethyl)aminehydrochloride (CAS: 25952-53-8, 1.309 g, 6.83 mmol). The mixture was stirred at rt for 20 min. To the mixture at 0° C. were added 2-aminoacetamide hydrochloride (CAS: 1668-10-6, 0.629 g, 5.69 mmol) and Et₃N (0.946 ml, 6.83 mmol). The mixture was stirred at rt for 5 h. The reaction mixture was concentrated, dissolved in EtOAc, washed with 2M aq. NaH₂PO₄, satd. aq. NaHCO₃, water and brine. The organic layer was dried over Na₂SO₄, filtered, and concentrated. The resulting residue was purified by silica gel flash column chromatography (gradient, heptane/EtOAc=100/0 to 0/100 then CH₂Cl₂/MeOH=100/0 to 90/10). Appropriate fractions were concentrated. To a solution of the obtained material (1.929 g, 3.89 mmol) in CH₂Cl₂ (6.5 ml) at 0° C. was added 4M HCl in MTHP (12.98 ml). The mixture was stirred at 0° C. for 1.5 h and rt for 0.5 h. To the reaction mixture at 0° C. were added satd. aq NaHCO₃ and CH₂Cl₂. The phases were separated, and the organic layer was extracted with 1M aq. NaHCO₃. The pH of the combined aqueous layer was adjusted to 4 with 2M aq. NaH₂PO₄ at 0° C. The mixture was extracted with EtOAc. The pH of the aqueous layer was adjusted to pH 1 with 1M aq. HCl. The mixture was extracted with EtOAc and EtOAc/THF (6/1 three times). The combined organic extracts were washed with brine, dried over Na₂SO₄, filtrated, and concentrated to afford the title compound (1.17 g, 2.60 mmol). ¹H NMR (500 MHz, DMSO-d₆) δ 12.89 (br, 1H), 8.06-7.99 (m, 1H), 7.92-7.88 (m, 2H), 7.69-7.61 (m, 2H), 7.45-7.39 (m, 2H), 7.37-7.27 (m, 2H), 7.26 (br, 1H), 7.06-6.98 (m, 1H), 4.52-4.43 (m, 1H), 4.35-4.22 (m, 3H), 3.65-3.54 (m, 2H), 2.81-2.76 (m, 3H), 2.19-2.06 (m, 3H), 1.93-1.83 (m, 1H). LC-MS (ESI, m/z): [M+H]⁺ 440.

### Example-3-12-06: (2S)-2-({[(9H-fluoren-9-yl)methoxy]carbonyl}amino)-4-{[(pyrimidin-5-yl)methyl]carbamoyl}butanoic acid

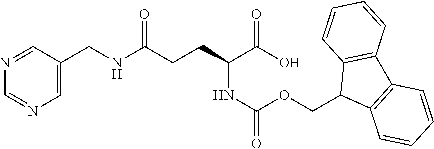 [SMILES: O=C(CC[C@H](NC(=O)OCC1c2ccccc2-c2ccccc21)C(=O)O)NCc1cncnc1]

To a solution of (4S)-5-(tert-butoxy)-4-({[(9H-fluoren-9-yl)methoxy]carbonyl}(methyl)amino)-5-oxopentanoic acid (CAS: 84793-07-7, 1.20 g, 2.82 mmol), 1-(pyrimidin-5-yl)methanamine hydrochloride (CAS: 1199773-53-9, 0.452 g, 3.10 mmol) and HATU (1.180 g, 3.10 mmol) in CH₂Cl₂ (9.4 mL) at rt was added DIPEA (1.458 g, 11.28 mmol). The reaction mixture was stirred at rt for 3 h. The mixture was concentrated and the resulting residue was purified by silica gel flash column chromatography (gradient, hexane/EtOAc=75/25 to 0/100). Appropriate fractions were concentrated. To a solution of the obtained material (1.457 g, 2.82 mmol) in CH₂Cl₂ (10 mL) at rt was added 4M HCl in EtOAc (10 mL). The resulting mixture was stirred at rt for 4 h. The mixture was concentrated and the residue was washed with CH₂Cl₂ to afford the title compound (1.10 g, 2.214 mmol). ¹H NMR (500 MHz, DMSO-d₆) δ 9.08 (s, 1H), 8.70 (s, 2H), 8.50 (t, J=5.8 Hz, 1H), 7.89 (d, J=7.6 Hz, 2H), 7.73 (d, J=7.5 Hz, 2H), 7.67 (d, J=8.1 Hz, 1H), 7.41 (t, J=7.4 Hz, 2H), 7.33 (t, J=7.1 Hz, 2H), 4.30-4.22 (m, 5H), 3.98-3.93 (m, 1H), 2.26 (t, J=7.7 Hz, 2H), 2.06-1.97 (m, 1H), 1.85-1.72 (m, 1H). LC-MS (ESI, m/z): [M+H]⁺ 461.

### Example-3-12-07: (2S)-4-{[(3R)-1-[(tert-butoxy)carbonyl]pyrrolidin-3-yl]carbamoyl}-2-({[(9H-fluoren-9-yl)methoxy]carbonyl}amino)butanoic acid

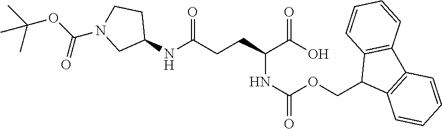 [SMILES: CC(C)(C)OC(=O)N1CC[C@@H](NC(=O)CC[C@H](NC(=O)OCC2c3ccccc3-c3ccccc32)C(=O)O)C1]

To a solution of (4S)-4-({[(9H-fluoren-9-yl)methoxy]carbonyl}amino)-5-oxo-5-(prop-2-en-1-yloxy)pentanoicacid (CAS: 144120-54-7, 20.00 g, 48.847 mmol) in DMF (800 mL) at rt were added HATU (37.15 g, 97.695 mmol), DIPEA (25.25 g, 263.329 mmol) and tert-butyl (3R)-3-aminopyrrolidine-1-carboxylate (CAS: 147081-49-0, 10.92 g, 58.617 mmol). The reaction mixture was stirred at rt for 1 h. The resulting solution was extracted with EtOAc three times. The combined organic extracts were washed with brine three times, dried over Na₂SO₄, filtered and concentrated. The resulting residue was purified by silica gel flash column chromatography (EtOAc/petroleum ether=1/5 to 1/2). Appropriate fractions were concentrated. To the resulting residue (40.9 g, 70.8 mmol) in THF (300 mL) at rt were added Pd(PPh₃)₄ (4.00 g, 3.462 mmol) and morpholine (CAS: 110-91-8, 6.09 mL, 69.242 mmol). The resulting mixture was stirred at rt for 0.5 min. The pH of the mixture was adjusted to pH 5 with citric acid. The mixture was extracted with EtOAc three times. The combined organic extracts were washed with brine three times, dried over Na₂SO₄, filtered, and concentrated. The resulting residue was purified by silica gel flash column chromatography (isocratic, CH₂Cl₂/MeOH=10/1), and then further purified by reversed phase silica gel flash column chromatography (C18 silica gel, gradient, H₂O with 0.1% TFA/CH₃CN=65/35 to 45/55) to afford the title compound (5.323 g, 9.90 mmol). ¹H NMR (400 MHz, DMSO-d₆) δ 12.62 (br, 1H), 8.09 (br, 1H), 7.90 (d, J=7.5 Hz, 2H), 7.74 (d, J=7.5 Hz, 2H), 7.70-7.60 (m, 1H), 7.42 (t, J=7.5 Hz, 2H), 7.38-7.30 (m, 2H), 4.33-4.12 (m, 4H), 3.99-3.90 (m, 1H), 3.46-3.37 (m, 1H), 3.32-3.16 (m, 2H), 3.09-2.94 (m, 1H), 2.25-2.11 (m, 2H), 2.05-1.88 (m, 2H), 1.85-1.62 (m, 2H), 1.39 (s, 9H). LC-MS (ESI, m/z): [M+H]⁺ 538.

### Example-3-12-08: (2S)-2-({[(9H-fluoren-9-yl)methoxy]carbonyl}amino)-3-(5,6,7,8-tetrahydronaphthalen-2-yl)propanoic acid

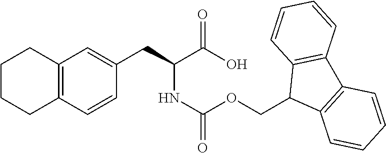 [SMILES: O=C(N[C@@H](Cc1ccc2c(c1)CCCC2)C(=O)O)OCC1c2ccccc2-c2ccccc21]

To a suspension of Zn (18.58 g, 284.185 mmol) in DMF (400 mL) at rt was added iodine (3.66 g, 14.421 mmol) and methyl (2R)-2-({[(9H-fluoren-9-yl)methoxy]carbonyl}amino)-3-iodopropanoate (CAS: 156017-42-4, 51.30 g, 113.689 mmol). The mixture was stirred at rt for 30 min. To the mixture at rt was added 6-bromo-1,2,3,4-tetrahydronaphthalene (CAS: 6134-56-1, 20.00 g, 94.741 mmol), SPhos (1.94 g, 4.726 mmol and Pd₂(dba)₃ (2.17 g, 2.370 mmol). The resulting solution at 50° C. was stirred overnight. The reaction mixture was diluted with H₂O and EtOAc. The mixture was filtered. The filtrate was extracted with EtOAc. The organic extract was washed with brine, dried over Na₂SO₄, filtered, and concentrated. The resulting residue was purified by silica gel flash column chromatography (EtOAc/petroleum ether=1/2). Appropriate fractions were concentrated. To a solution of the obtained material (5.00 g, 10.977 mmol) in 2-propanol (200 mL) and H₂O (100 mL) at 0° C. were added CaCl₂ (69.0 g, 622 mmol) and NaOH (0.88 g, 22.002 mmol). The mixture was stirred at rt overnight. The resulting mixture was filtered, the pH of the filtrate was adjusted to pH 7 with aq. citric acid. The resulting mixture was extracted three times with EtOAc. The combined organic extracts were washed three times with H₂O, dried over Na₂SO₄, filtered, and concentrated. The resulting residue was triturated with CH₃CN and the solid was collected by filtration to afford the title compound (2.20 g, 4.99 mmol). ¹H NMR (500 MHz, DMSO-d₆) δ 7.86 (d, J=7.5 Hz, 2H), 7.68-7.49 (m, 2H), 7.43-7.34 (m, 2H), 7.33-7.21 (m, 2H), 6.92-6.85 (m, 1H), 6.85-6.79 (m, 2H), 6.78-6.66 (m, 1H), 4.38-4.20 (m, 1H), 4.20-3.92 (m, 3H), 3.19-3.14 (m, 1H), 3.13-3.01 (m, 1H), 2.94-2.74 (m, 1H), 2.58-2.52 (m, 3H), 1.61 (br, 4H). LC-MS (ESI, m/z): [M−H]⁻ 440.

### Example-3-13-01: (2S)-3-{5-[2-(tert-butoxy)-2-oxoethoxy]-1-[(tert-butoxy)carbonyl]-1H-indol-3-yl}-2-({[(9H-fluoren-9-yl)methoxy]carbonyl}amino)propanoic acid

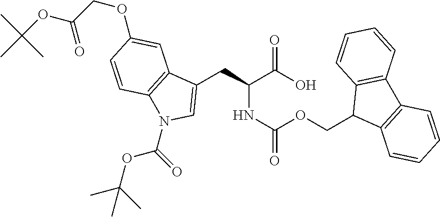 [SMILES: CC(C)(C)OC(=O)COc1ccc2c(c1)c(C[C@H](NC(=O)OCC1c3ccccc3-c3ccccc31)C(=O)O)cn2C(=O)OC(C)(C)C]

To a stirred mixture of 1H-indol-5-ol (CAS: 1953-54-4, 10 g, 75.1 mmol) and K₂CO₃ (20.8 g, 150 mmol) in DMF (200 mL) was added tert-butyl 2-bromoacetate (CAS: 5292-43-3, 14.6 g, 75.1 mmol) dropwise at rt, then stirred at rt for overnight. The resulting mixture was diluted with H₂O and extracted with EtOAc. The combined organic layers were washed with brine, dried over Na₂SO₄, filtered, and then concentrated. To a stirred mixture of the resulting residue (35.0 g, 142 mmol) and KOH (15.9 g, 283 mmol) in DMF (300 mL) was added I₂ (39.5 g, 156 mmol) in portions at rt, then stirred at rt for 2 h. The resulting mixture was diluted with H₂O and extracted with EtOAc. The combined organic layers were washed with brine, dried over Na₂SO₄, After filtration, filtered, and concentrated. A solution of the resulting residue (50.0 g, 134.0 mmol) in CH₂Cl₂ (500 mL) were added DMAP (3.27 g, 26.8 mmol) and Boc₂O (43.8 g, 201 mmol). The mixture was stirred for overnight at rt. The resulting mixture was washed with brine, dried over Na₂SO₄, filtered, and concentrated. The residue was purified by silica gel flash column chromatography (petroleum ether/EtOAc=1/1). Appropriate fractions were concentrated. To a suspension of Zn (18.2 g, 279 mmol) in DMF (600 mL) was added I₂ (3.54 g, 13.9 mmol). The mixture was stirred at rt for 10 min. To the mixture at rt were added methyl (2R)-2-[[(9H-fluoren-9-ylmethoxy)carbonyl]amino]-3-iodopropanoate (CAS: 156017-42-4, 7.30 g, 16.2 mmol) and 12 (3.54 g, 13.9 mmol). The mixture was stirred at rt for an additional 30 min. To the mixture were added the obtained material in the last step (44.0 g, 93.0 mmol), Pd₂(dba)₃·CHCl₃ (2.85 g, 2.79 mmol), and SPhos (1.91, 4.65 mmol). The mixture was stirred at 60° C. for an additional 2 h. The resulting mixture was diluted with H₂O. The mixture was extracted three times with EtOAc. The combined organic extracts were washed with brine, dried over Na₂SO₄, filtered, and then concentrated. The resulting residue was purified by silica gel flash column chromatography (isocratic, petroleum ether/EtOAc=50/50). Appropriate fractions were concentrated. To a mixture of the obtained material (40.0 g, 59.6 mmol) in 2-propanol (1200 mL) at 0° C. were added CaCl₂ (106 g, 954 mmol) and LiOH·H₂O (10.00 g, 239 mmol) in water (400 mL) dropwise at 0° C. The mixture was stirred at rt for 2 h and filtered. The pH of the mixture was adjusted to 6 with aq. citric acid. The resulting mixture was extracted three times with EtOAc. The combined organic extracts were washed three times with brine, dried over Na₂SO₄, filtered, and then concentrated. The resulting residue was purified by reversed phase silica gel flash column chromatography (C18 silica gel, gradient, H₂O/CH₃CN=90/10 to 50/50) to afford the title compound (26.7 g, 40.6 mmol). ¹H NMR (300 MHz, DMSO-d₆) δ 7.92-7.86 (m, 3H), 7.63-7.58 (m, 3H), 7.52 (m, 1H), 7.41-7.36 (m, 3H), 7.30-7.14 (m, 3H), 6.96-6.92 (m, 1H), 4.66 (s, 2H), 4.25-4.18 (m, 3H), 3.42-2.94 (m, 2H), 1.55 (s, 9H), 1.42 (s, 9H). LC-MS (ESI, m/z): [M−H]⁻ 655.

### Example-3-13-02: (2S)-2-({[(9H-fluoren-9-yl)methoxy]carbonyl}amino)-3-{3′-methanesulfonyl-[1,1′-biphenyl]-4-yl}propanoic acid

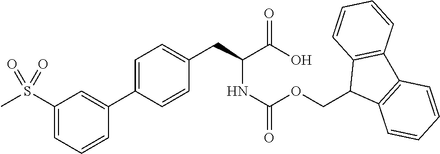 [SMILES: CS(=O)(=O)c1cccc(-c2ccc(C[C@H](NC(=O)OCC3c4ccccc4-c4ccccc43)C(=O)O)cc2)c1]

To a solution of (2S)-3-(4-bromophenyl)-2-[[(tert-butoxy)carbonyl]amino]propanoic acid (CAS: 62129-39-9, 17.0 g, 49.4 mmol) in DME/H₂O (3/1, 400 mL) were added (3-methanesulfonylphenyl)boronic acid (CAS: 373384-18-0, 10.0 g, 50.0 mmol), Pd(dppf)Cl₂ (1.1 g, 1.5 mmol) and Na₂CO₃ (9.3 g, 87.7 mmol). The resulting solution was stirred overnight at 90° C. The pH value of the solution was adjusted to 4-5 with aq. citric acid. The resulting solution was extracted with EtOAc and the combined organic layers were dried over Na₂SO₄, filtered, and then concentrated. The residue was purified by silica gel flash column chromatography (isocratic, CH₂Cl₂/MeOH=10/1). Appropriate fractions were concentrated. To a solution of the obtained material (10 g, 23.8 mmol) in CH₂C12 (300 mL) was added TFA (60 mL). The resulting solution was stirred for 2 h at rt. The resulting mixture was concentrated under vacuum. To a solution of the resulting residue (15.0 g, 47.0 mmol) in 1,4-dioxane (200 mL) and H₂O (200 mL) at 0° C. was added NaHCO₃ (11.8 g, 141 mmol), followed by the addition of Fmoc-OSu (20.6 g, 61.1 mmol) in portions. The mixture was stirred overnight at rt, and then pH of the solution was adjusted to 6 with aq. citric acid. The mixture was extracted with EtOAc. The organic extracts were washed with brine, dried over Na₂SO₄, filtered, and concentrated. The resulting residue was purified by reversed phase silica gel flash column chromatography (C18 silica gel, gradient, H₂O/CH₃CN=70/30 to 20/80) to afford the title compound (4.00 g, 7.38 mmol). ¹H NMR (300 MHz, DMSO-d₆) δ 12.80 (br, 1H), 8.13 (m, 1H), 8.00-7.94 (m, 1H), 7.91-7.86 (m, 3H), 7.80-7.63 (m, 6H), 7.47-7.37 (m, 4H), 7.32-1.25 (m, 2H), 4.27-4.14 (m, 4H), 3.29 (s 3H), 3.19-3.13 (m, 1H), 2.98-2.90 (m, 1H). LC-MS (ESI, m/z): [M−H]⁻ 540.

### Example-3-13-03: (2S)-2-({[(9H-fluoren-9-yl)methoxy]carbonyl}amino)-3-methanesulfonamidopropanoic acid

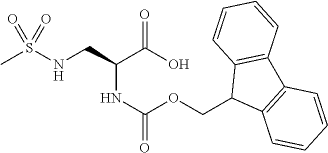 [SMILES: CS(=O)(=O)NC[C@H](NC(=O)OCC1c2ccccc2-c2ccccc21)C(=O)O]

To a solution of (2S)-3-amino-2-[[(9H-fluoren-9-ylmethoxy) carbonyl]amino]propanoic acid (CAS: 181954-34-7, 15.00 g, 45.96 mmol) in MeOH (150 mL) was added SOCl₂ (16.40 g, 137.9 mmol) dropwise with stirring at 0° C. The resulting solution was stirred for 3 hr at rt. The resulting mixture was concentrated. To a solution of the resulting residue (13.30 g, 39.1 mmol) in THF (130 mL) were added Et₃N (11.86 g, 117.2 mmol) and MsCl (6.71 g, 58.6 mmol) dropwise with stirring at 0° C. The resulting solution was stirred for 1 hr at rt. The reaction was then quenched with water. The resulting solution was extracted with EtOAc and the combined organic layer was washed with brine, dried over Na₂SO₄ and concentrated. To a mixture of the obtained material (13.0 g, 31.1 mmol) in 2-propanol (300 mL) and H₂O (100 mL) at 0° C. were added CaCl₂ (55.2 g, 497 mmol) and LiOH·H₂O (2.98 g, 124 mmol). The mixture was stirred at rt for 8 h. The pH of the mixture was adjusted to pH 5 with 3M aq. HCl. The resulting mixture was extracted three times with EtOAc. The combined organic extracts were washed three times with H₂O, dried over Na₂SO₄, filtered, and then concentrated. The resulting residue was purified by reversed phase silica gel flash column chromatography (C18 silica gel, gradient, 0.1% formic acid in H₂O/CH₃CN=50/50 to 30/70) to afford the title compound (5.07 g, 12.5 mmol). ¹H NMR (300 MHz, DMSO-d₆) δ 12.91 (s, 1H), 7.90-7.88 (m, 2H), 7.73-7.71 (m, 2H), 7.57-7.54 (m, 1H), 7.47-7.31 (m, 4H), 7.15-7.11 (m, 1H), 4.40-4.06 (m, 4H), 3.43-3.18 (m, 2H), 2.89 (s, 3H). LC-MS (ESI, m/z): [M−H]⁻ 403.

### Example-3-13-04: (2S)-3-(2-acetamidoacetamido)-2-({[(9H-fluoren-9-yl)methoxy]carbonyl}(methyl)amino)propanoic acid

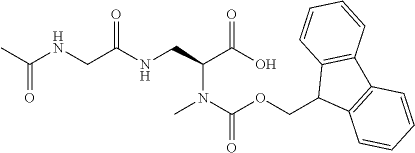 [SMILES: CC(=O)NCC(=O)NC[C@@H](C(=O)O)N(C)C(=O)OCC1c2ccccc2-c2ccccc21]

To a solution of (S)-2-((((9H-fluoren-9-yl)methoxy)carbonyl)(methyl)amino)-3-((tert-butoxycarbonyl)amino)propanoic acid (CAS: 1424972-75-7, 5 g, 11.35 mmol) in DMF (22.70 ml) were added DIPEA (2.97 ml, 17.03 mmol) and allyl bromide (CAS: 106-95-6, 1.277 ml, 14.76 mmol) at rt. The mixture was stirred at rt for overnight. After diluted by EtOAc and Hexane, the organic extract was washed by water, 0.25 M aq. citric acid, sat. aq. NaHCO₃, and water. The organic layer was dried over Na₂SO₄, filtered, and concentrated. The resulting residue was purified by silica gel flash column chromatography (gradient, EtOAc/hexane=0/100 to 50/50). Appropriate fractions were concentrated. To a solution of the resulting residue in CH₂Cl₂ was added 4M aq. HCl in 1,4-dioxane (20 ml, 80 mmol) at rt. After stirred for 2 h, this mixture was concentrated. To a solution of the resulting residue (2.50 g, 6.00 mmol) and 2-acetamidoacetic acid (CAS: 543-24-8, 0.843 g, 7.20 mmol) in DMF (24 ml) were added DIPEA (2.62 ml, 14.99 mmol) and HATU (2.74 g, 7.20 mmol) at 0° C. The mixture was stirred at rt for 2 h. After diluted with EtOAc and hexane, the organic extract was washed by 1M aq. HCl, satd. aq. NaHCO₃, and brine. The organic layer was dried over Na₂SO₄, filtered, and concentrated. To a solution of the resulting residue in CH₂Cl₂ (50 ml) was added phenylsilane (CAS: 694-53-1, 1.478 ml, 11.99 mmol) and Pd(PPh₃)₄ (0.173 g, 0.150 mmol) at 0° C. and the resulting mixture was stirred for 1 h at the same temperature. After concentrated, The resulting residue was purified by silica gel flash column chromatography (gradient, CH₂Cl₂/MeOH=100/0 to 70/30) to give the title compound (1.4996 g, 3.41 mmol). ¹H NMR (500 MHz, DMSO-d₆) δ 8.12-7.98 (m, 2H), 7.92-7.88 (m, 2H), 7.67-7.64 (m, 2H), 7.44-7.40 (m, 2H), 7.36-7.30 (m, 2H), 4.30-4.09 (m, 4H), 3.67-3.53 (m, 3H), 3.17-3.16 (m, 1H), 2.85-2.82 (m, 3H), 1.91-1.83 (s, 3H). LC-MS (ESI, m/z): [M+H]⁺ 440.

### Example-3-13-05: (2S)-2-({[(9H-fluoren-9-yl)methoxy]carbonyl}amino)-3-[3-({[(prop-2-en-1-yloxy)carbonyl]amino}methyl)phenyl]propanoic acid

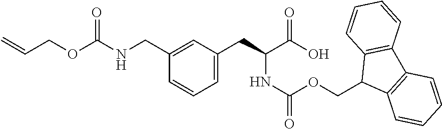 [SMILES: C=CCOC(=O)NCc1cccc(C[C@H](NC(=O)OCC2c3ccccc3-c3ccccc32)C(=O)O)c1]

To a solution of 1-(3-bromophenyl)methanamine (CAS: 10269-01-9, 20 g, 108 mmol) in CH₂Cl₂ (200 mL) was added Et₃N (22.4 mL, 161 mmol) and Boc₂O (25.8 g, 118 mmol) at 0° C. The resulting solution was allowed to warm to rt and stirred for 1 h. The resulting mixture was washed three times with brine, dried over Na₂SO₄, filtered, and then concentrated. The resulting residue was purified by silica gel flash column chromatography (isocratic, petroleum ether/EtOAc=15/1). Appropriate fractions were concentrated. To a suspension of Zn (41.5 g, 635 mmol) in DMF (200 mL) at rt were added 1,2-dibromoethane (1.82 mL, 21.1 mmol) and chlorotrimethylsilane (1.34 mL, 10.5 mmol). The mixture was stirred at 60° C. for 30 min. To the mixture at rt was added methyl (R)-2-((((9H-fluoren-9-yl)methoxy)carbonyl)amino)-3-iodopropanoate (CAS: 156017-42-4, 57.3 g, 127 mmol). The mixture was stirred at 35° C. for 1 h. To a mixture of the obtained material in the last step (30.3 g, 106 mmol), Pd₂(dba)₃ CHCl₃ adduct (4.84 g, 5.29 mmol), and P(o-Tol)₃ (6.45 g, 21.2 mmol) in DMF (200 mL) at 50° C. was added the organozinc reagent. The mixture was stirred overnight at 50° C. The mixture was filtered. The filtrate was diluted with H₂O and extracted with EtOAc. The organic extract was washed with brine, dried over Na₂SO₄, filtered, and then concentrated. The resulting residue was purified by silica gel flash column chromatography (isocratic, petroleum ether/EtOAc=10/1). Appropriate fractions were concentrated. To a solution of the obtained material (39.5 g, 74.4 mmol) in CH₂Cl₂ (200 mL) was added TFA (100 mL). The resulting solution was stirred overnight at rt. The resulting mixture was concentrated under vacuum. To a solution of the resulting residue (32.0 g, 60.6 mmol) in CH₂Cl₂ (200 mL) was added dropwise Et₃N (9.18 g, 90.9 mmol) and allyl chlorocarbonate (8.0 g, 66.4 mmol) at 0° C., then the mixture was allowed to warm to rt and stirred for 2 h. The resulting mixture was washed with brine, dried over dried over Na₂SO₄, filtered, and then concentrated. To a mixture of the obtained material (20.0 g, 38.8 mmol) in 2-propanol (300 mL) and H₂O (100 mL) at 0° C. were added CaCl₂ (43.1 g, 389 mmol) and NaOH (3.11 g, 77.8 mmol). The mixture was stirred at rt for 5 h. The mixture was filtered. The pH of the mixture was adjusted to 7 with aq. citric acid. The resulting mixture was extracted three times with EtOAc. The combined organic extracts were washed three times with H₂O, dried over Na₂SO₄, filtered, and then concentrated. The resulting residue was purified by silica gel flash column chromatography (gradient, petroleum ether/EtOAc=4/1) to afford the title compound (5.67 g, 11.3 mmol). ¹H NMR (300 MHz, DMSO-d₆) δ 12.74 (s, 1H), 7.89-7.86 (m, 2H), 7.76-7.63 (m, 3H), 7.43-7.33 (m, 2H), 7.31-7.09 (m, 6H), 5.97-5.84 (m, 1H), 5.30-5.14 (m, 2H), 4.49-4.47 (m, 2H), 4.23-4.15 (m, 6H), 3.08-3.04 (m, 1H), 2.94-2.78 (m, 1H). LC-MS (ESI, m/z): [M+H]⁺ 501.

### Example-3-13-06: (2S)-2-({[(9H-fluoren-9-yl)methoxy]carbonyl}(methyl)amino)-3-(4-{[(prop-2-en-1-yloxy)carbonyl]amino}phenyl)propanoic acid

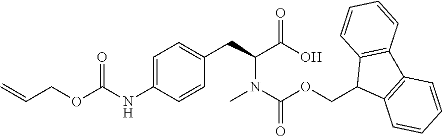 [SMILES: C=CCOC(=O)Nc1ccc(C[C@@H](C(=O)O)N(C)C(=O)OCC2c3ccccc3-c3ccccc32)cc1]

To a solution of (2S)-2-[[(9H-fluoren-9-ylmethoxy)carbonyl]amino]-3-(4-[[(prop-2-en-1-yloxy)carbonyl]amino]phenyl)propanoic acid (CAS: 220848-55-5, 10.00 g, 20.6 mmol) in toluene (200 mL) at rt were added acetic acid (2.00 mL), CSA (0.95 g, 4.11 mmol) and paraformaldehyde (6.17 g, 205 mmol). The mixture was stirred at 95° C. for 35 min, and then concentrated. The resulting residue was purified by silica gel flash column chromatography (gradient, petroleum ether/EtOAc=100/1 to 2/1). Appropriate fractions were concentrated. To the resulting residue (7.00 g, 14.0 mmol) in CHCl₃ (35 mL) were added triethylsilane (35.0 ml, 217 mmol) and TFA (35.0 mL, 307 mmol). The mixture was stirred at rt for 2 days and then concentrated. The resulting residue was purified by reversed phase silica gel flash column chromatography (silica gel, gradient, 0.1% formic acid in H₂O/CH₃CN=55/45 to 38/62) to afford the title compound (3.85 g, 7.69 mmol). ¹H NMR (400 MHz, Methanol-d₄) δ 7.80-7.77 (m, 2H), 7.56-7.27 (m, 9H), 7.14-7.12 (m, 1H), 6.89-6.86 (m, 1H), 5.99-5.96 (m, 1H), 5.37-5.32 (m, 1H), 5.23-5.20 (m, 1H), 4.88-4.82 (m, 1H), 4.62-4.59 (m, 2H), 4.36-4.14 (m, 3H), 3.31-3.30 (m, 1H), 3.06-3.03 (m, 1H), 2.76-2.72 (m, 3H). LC-MS (ESI, m/z): [M+H]⁺ 501.

### Example-3-14

The following compounds can be synthesized as outlined for the preparation of Reference example-3-1-04, Example-3-2-06, Example-3-3-05 or Example-3-7-04 employing appropriate starting materials in the table.

| | Structure and IUPAC name |
|---|---|
| | Starting material-A |
| | Starting material-B |
| Example | NMR and MS |
| 3-14-01 | 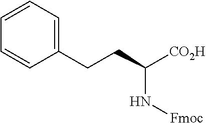 |
| | (2S)-2-({[(9H-fluoren-9-yl)methoxy]carbonyl}amino)- |
| | 4-phenylbutanoicacid |
| | Reference example-3-1-03 |
| | bromobenzene (CAS: 108-86-1) |
| | ¹H NMR (500 MHz, DMSO-d₆) δ 12.64 (br, 1H), 7.95-7.87 |
| | (m, 2H), 7.81-7.56 (m, 3H), 7.46-7.38 (m, 2 H), 7.38-7.25 |
| | (m, 4H), 7.25-7.16 (m, 3H), 4.40-4.13 (m, 3H), 3.99-3.87 |
| | (m, 1H), 2.73-2.55 (m, 2H), 2.04-1.81 (m, 2H). |
| | LC-MS (ESI, m/z): [M + H]⁺ 402. |
| 3-14-02 | 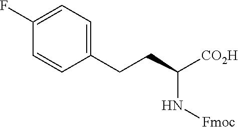 |
| | (2S)-2-({[(9H-fluoren-9-yl)methoxy]carbonyl} |
| | amino)-4-(4-fluorophenyl)butanoic acid |
| | Reference example-3-1-03 |
| | 1-bromo-4-fluorobenzene (CAS: 460-00-4) |
| | ¹H NMR (500 MHZ, DMSO-d₆) δ 12.76 (br, 1H), 7.95-7.85 |
| | (m, 2H), 7.88-7.56 (m, 3H), 7.46-7.17 (m, 6H), 7.15-7.05 |
| | (m, 2H), 4.41-4.13 (m, 3H), 3.93-3.82 (m, 1H), 2.72-2.46 |
| | (m, 2H), 2.04-1.77 (m, 2H). LC-MS (ESI, m/z): |
| | [M + H]⁺ 420. |
| 3-14-03 | 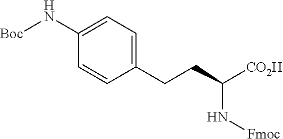 |
| | (2S)-4-(4-{[(tert-butoxy)carbonyl]amino}phenyl)-2-({[(9H- |
| | fluoren-9-yl)methoxy]carbonyl}amino)butanoic acid |
| | Reference example-3-1-03 |
| | tert-butyl N-(4-bromophenyl)carbamate |
| | (CAS: 131818-17-2) |
| | ¹H NMR (500 MHz, DMSO-d₆) δ 12.58 (br, 1H), 9.25 |
| | (s, 1H), 7.95-7.85 (m, 2H), 7.79-7.16 (m, 9H), 7.13-7.01 |
| | (m, 2H), 4.37-4.12 (m, 3H), 3.94-3.83 (m, 1H), 2.68-2.57 |
| | (m, 1H), 2.56-2.54 (m, 1H), 1.98-1.77 (m, 2H), 1.46 (s, 9H). |
| | LC-MS (ESI, m/z): [M + H]⁺ 517. |
| 3-14-04 | 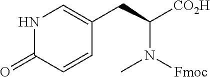 |
| | (2S)-2-({[(9H-fluoren-9-yl)methoxy]carbonyl}(methyl) |
| | amino)-3-(6-oxo-1,6-dihydropyridin-3-yl)propanoic acid |
| | Reference example-3-1-01 |
| | 5-bromo-2-(methoxymethoxy)pyridine |
| | (CAS: 247189-74-8) |
| | ¹H NMR (500 MHz, DMSO-d₆) δ 12.88 (br, 1H), 11.43 |
| | (br, 1H), 7.89 (d, J = 7.6 Hz, 2H), 7.62-7.49 (m, 2H), |
| | 7.46-7.37 (m, 2H), 7.36-7.03 (m, 4H), 6.25-6.17 (m, 1H), |
| | 4.77-4.50 (m, 1H), 4.38-4.09 (m, 3H), 2.95-2.56 (m, |
| | 5H). LC-MS (ESI, m/z): [M + H]⁺ 419. |
| 3-14-05 | 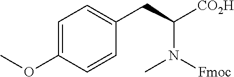 |
| | (2S)-2-({[(9H-fluoren-9-yl)methoxy]carbonyl}(methyl) |
| | amino)-3-(4-methoxyphenyl)propanoic acid |
| | Reference example-3-1-01 |
| | 1-bromo-4-methoxybenzene (CAS: 104-92-7) |
| | ¹H NMR (500 MHz, DMSO-d₆) δ 12.89 (br, 1H), 7.92-7.85 |
| | (m, 2H), 7.60-7.38 (m, 4H), 7.36-7.26 (m, 2H), 7.15-6.93 |
| | (m, 2H), 6.84-6.74 (m, 2H), 4.79-4.59 (m, 1H), 4.35-4.14 |
| | (m, 3H), 3.73-3.63 (m, 3H), 3.19-2.75 (m, 2H), 2.73-2.62 |
| | (m, 3H). LC-MS (ESI, m/z): [M + H]⁺ 432. |
| 3-14-06 | 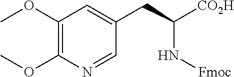 |
| | (2S)-3-(5,6-dimethoxypyridin-3-yl)-2-({[(9H- |
| | fluoren-9-yl)methoxy]carbonyl}amino)propanoic acid |
| | methyl (2R)-2-({[(9H-fluoren-9-yl)methoxy]carbonyl} |
| | amino)-3-iodopropanoate (CAS: 156017-42-4) |
| | 5-bromo-2,3-dimethoxypyridine (CAS: 52605-98-8) |
| | ¹H NMR (500 MHZ, DMSO-d₆) δ 12.75 (br, 1H), 7.96-7.84 |
| | (m, 2H), 7.81-7.21 (m, 9H), 4.32-4.09 (m, 4H), 3.86-3.69 |
| | (m, 6H), 3.08-2.97 (m, 1H), 2.86-2.77 (m, 1H). |
| | LC-MS (ESI, m/z): [M + H]⁺ 449. |
| 3-14-07 | 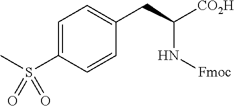 |
| | (2S)-2-({[(9H-fluoren-9-yl)methoxy]carbonyl}amino)- |
| | 3-(4-methanesulfonylphenyl)propanoic acid |
| | methyl (2R)-2-({[(9H-fluoren-9-yl)methoxy]carbonyl} |
| | amino)-3-iodopropanoate (CAS: 156017-42-4) |
| | 1-bromo-4-methanesulfonylbenzene (CAS: 3466-32-8) |
| | ¹H NMR (500 MHz, DMSO-d₆) δ 12.86 (br, 1H), 7.92-7.72 |
| | (m, 5H), 7.68-7.59 (m, 2H), 7.57-7.46 (m, 2H), 7.44-7.37 |
| | (m, 2H), 7.34-7.24 (m, 2H), 4.32-4.09 (m, 4H), 3.26-3.08 |
| | (m, 4H), 3.03-2.81 (m, 1H). LC-MS (ESI, |
| | m/z): [M + H]⁺ 466. |
| 3-14-08 | 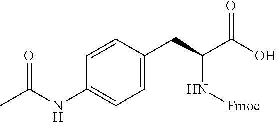 |
| | (2S)-3-(4-acetamidophenyl)-2-({[(9H-fluoren-9- |
| | yl)methoxy]carbonyl}amino)propanoic acid |
| | methyl (2R)-2-({[(9H-fluoren-9-yl)methoxy]carbonyl} |
| | amino)-3-iodopropanoate (CAS: 156017-42-4) |
| | N-(4-bromophenyl)acetamide (CAS: 103-88-8) |
| | ¹H NMR (500 MHZ, DMSO-d₆) δ 12.73-12.70 (m, 1H), |
| | 9.92-9.82 (m, 1H), 7.88 (d, J = 7.5 Hz, 2H), 7.72 (d, J = |
| | 7.0 Hz, 1H), 7.68-7.61 (m, 2H), 7.53-7.45 (m, 2H), 7.44- |
| | 7.37 (m, 2H), 7.35-7.23 (m, 2H), 7.21-7.13 (m, 2H), 4.25- |
| | 4.07 (m, 4H), 3.06-2.93 (m, 1H), 2.85-2.76 (m, 1H), 2.02 |
| | (s, 3H). LC-MS (ESI, m/z): [M + H]⁺ 445. |
| 3-14-09 | 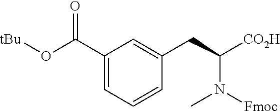 |
| | (2S)-3-{3-[(tert-butoxy)carbonyl]phenyl}-2-({[(9H- |
| | fluoren-9-yl)methoxy]carbonyl}(methyl)amino) |
| | propanoic acid |
| | Reference example-3-1-01 |
| | tert-butyl 3-bromobenzoate (CAS: 69038-74-0) |
| | ¹H NMR (500 MHz, DMSO-d₆) δ 12.99 (br, 1H), 7.92-7.84 |
| | (m, 2H), 7.83-7.78 (m, 1H), 7.77-7.68 (m, 1H), 7.66-7.20 |
| | (m, 8H), 4.90-4.77 (m, 1H), 4.28-4.08 (m, 3H), 3.31-2.96 |
| | (m, 2H), 2.72 (s, 3H), 1.55-1.38 (m, 9H). |
| | LC-MS (ESI, m/z): [M − tBu + H]⁺ 446. |
| 3-14-10 | 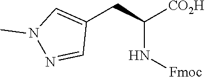 |
| | (2S)-2-({[(9H-fluoren-9-yl)methoxy]carbonyl}amino)- |
| | 3-(1-methyl-1H-pyrazol-4-yl)propanoic acid |
| | methyl (2R)-2-({[(9H-fluoren-9-yl)methoxy]carbonyl} |
| | amino)-3-iodopropanoate (CAS: 156017-42-4) |
| | 4-bromo-1-methyl-1H-pyrazole (CAS: 15803-02-8) |
| | ¹H NMR (500 MHZ, DMSO-d₆) δ 12.73 (br, 1H), 7.89 |
| | (d, J = 7.63 Hz, 2H), 7.76-7.52 (m, 3H), 7.48-7.38 |
| | (m, 3H), 7.36-7.27 (m, 2H), 7.26 (s, 1H), 4.28-4.17 (m, 3H), |
| | 4.06-3.97 (m, 1H), 3.77-3.70 (m, 3H), 2.91-2.84 (m, |
| | 1H), 2.72-2.67 (m, 1H). LC-MS (ESI, m/z): [M + H]⁺ 392 |
| 3-14-11 | 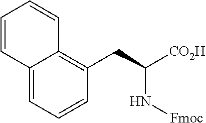 |
| | (2S)-2-({[(9H-fluoren-9-yl)methoxy]carbonyl}amino)-3- |
| | (naphthalen-1-yl)propanoic acid |
| | methyl (2R)-2-({[(9H-fluoren-9-yl)methoxy]carbonyl} |
| | amino)-3-iodopropanoate (CAS: 156017-42-4) |
| | 1-bromonaphthalene (CAS: 90-11-9) |
| | ¹H NMR (400 MHZ, DMSO-d₆) δ 12.92 (br, 1H), 8.20-8.08 |
| | (m, 1H), 8.01-7.77 (m, 5H), 7.68-7.23 (m, 10H), 4.40-4.29 |
| | (m, 1H), 4.23-4.11 (m, 3H), 3.75-3.62 (m, 1H), 3.30-3.23 |
| | (m, 1H). LC-MS (ESI, m/z): [M − H]⁻ |
| | 436. |
| 3-14-12 | 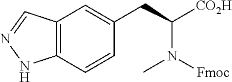 |
| | (2S)-2-({[(9H-fluoren-9-yl)methoxy]carbonyl}(methyl) |
| | amino)-3-(1H-indazol-5-yl)propanoic acid |
| | Reference example-3-1-01 |
| | 5-bromo-1H-indazole (CAS: 53857-57-1) |
| | ¹H NMR (500 MHZ, DMSO-d₆) δ 13.05-12.84 (m, 2H), |
| | 7.95 (br, 1H), 7.89-7.84 (m, 2H), 7.59-7.53 (m, 1H), |
| | 7.50-7.33 (m, 5H), 7.31-7.03 (m, 3H), 4.88-4.66 (m, 1H), |
| | 4.33-4.06 (m, 3H), 3.26-2.84 (m, 2H), 2.74-2.69 (m, |
| | 3H). LC-MS (ESI, m/z): [M + H]⁺ 442. |
| 3-14-13 | 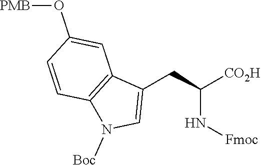 |
| | (2S)-3-{1-[(tert-butoxy)carbonyl]-5-[(4-methoxyphenyl) |
| | methoxy]-1H-indol-3-yl}-2-({[(9H-fluoren-9-yl)methoxy] |
| | carbonyl}amino)propanoic acid |
| | methyl (2R)-2-({[(9H-fluoren-9-yl)methoxy]carbonyl}amino)- |
| | 3-iodopropanoate (CAS: 156017-42-4) |
| | tert-butyl 3-bromo-5-[(4-methoxyphenyl)methoxy]-1H- |
| | indole-1-carboxylate |
| | ¹H NMR (500 MHZ, DMSO-d₆) δ 12.86 (br, 1H), 7.93-7.85 |
| | (m, 3H), 7.84-7.79 (m, 1H), 7.65-7.55 (m, 2H), 7.53 (s, 1H), |
| | 7.41-7.35 (m, 4H), 7.33-7.18 (m, 3H), 7.01-6.95 (m, 1H), |
| | 6.92 (d, J = 8.70 Hz, 2H), 5.05 (s, 2H), 4.33-4.25 (m, 1H), |
| | 4.22-4.15 (m, 3H), 3.79-3.67 (m, 3H), 3.20-3.11 (m, |
| | 1H), 3.05-2.96 (m, 1H), 1.68-1.44 (m, 9H). LC-MS |
| | (ESI, m/z): [M + H]⁺ 663. |

### Example-3-15-01: (2S)-4-butoxy-2-({[(9H-fluoren-9-yl)methoxy]carbonyl}amino)butanoic acid

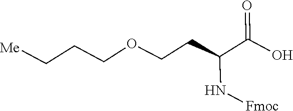

The solution of Reference example-3-2-03 (700 mg, 1.52 mmol) and sodium dihydrogen phosphate (CAS: 7558-80-7, 731 mg, 6.09 mmol) in 1-butanol (3.80 mL) at 25° C. was stirred for 2 h at 45° C. The resulting mixture was concentrated in vacuo. The residue was dissolved with EtOAc and water. The resulting mixture was extracted with EtOAc. The combined organic extracts were washed with brine, dried over Na₂SO₄, filtered, and then concentrated. The resulting residue was purified by flash column chromatography (gradient, heptane/EtOAc=90/10 to 60/40). Appropriate fractions were concentrated. To a solution of the residue (635.7 mg, 1.402 mmol) in DCM (2.63 mL) at 0° C. was added TFA (0.88 mL). The mixture was stirred at 25° C. for 1.5 h, and then concentrated. The residue was purified by flash column chromatography (gradient, DCM/MeOH=100/0 to 95/5) to afford the title compound (0.52 g, 1.29 mmol). ¹H NMR (500 MHz, DMSO-d₆) δ 12.60 (br, 1H), 7.90 (d, J=7.5 Hz, 2H), 7.77-7.68 (m, 2H), 7.66-7.54 (m, 1H), 7.47-7.38 (m, 2H), 7.37-7.27 (m, 2H), 4.33-4.17 (m, 3H), 4.11-4.00 (m, 1H), 3.46-3.24 (m, 4H), 2.02-1.92 (m, 1H), 1.82-1.70 (m, 1H), 1.52-1.40 (m, 2H), 1.35-1.23 (m, 2H), 0.92-0.79 (m, 3H). LC-MS (ESI, m/z): [M+H]⁺ 398.4.

### Example-3-15

The following compounds can be synthesized as outlined for the preparation of Reference example-3-3-01 or Example-3-15-01 employing appropriate starting materials in the table.

| | Structure and IUPAC name |
|---|---|
| | Starting material-A |
| Ex- | Starting material-B |
| ample | NMR and MS |
| 3-15- 02 | 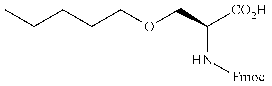 |
| | (2S)-2-({[(9H-fluoren-9-yl)methoxy]carbonyl}amino)-3- |
| | (pentyloxy)propanoic acid |
| | Reference example-3-2-01 |
| | pentan-1-ol (CAS: 71-41-0) |
| | ¹H NMR (500 MHz, DMSO-d₆) δ 12.81 (br, 1H), 7.90 (d, J = 7.6 |
| | Hz, 2H), 7.81-7.69 (m, 2H), 7.67-7.52 (m, 1H), 7.46-7.38 (m, 2H), |
| | 7.36-7.24 (m, 2H), 4.36-4.09 (m, 4H), 3.68-3.55 (m, 2H), 3.44-3.34 |
| | (m, 2H), 1.53-1.42 (m, 2H), 1.31-1.20 (m, 4H), 0.89-0.79 (m, 3H). |
| | LC-MS (ESI, mz): [M + H]⁺ 398. |
| 3-15- 03 | 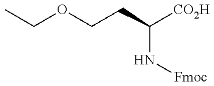 |
| | (2S)-4-ethoxy-2-({[(9H-fluoren-9-yl)methoxy]carbonyl}amino) |
| | butanoic acid |
| | Reference example-3-2-03 |
| | ethanol (CAS: 925-93-9) |
| | ¹H NMR (500 MHz, DMSO-d₆) δ 12.60 (br, 1H), 7.90 (d, J = 7.5 |
| | Hz, 2H), 7.76-7.68 (m, 2H), 7.65-7.58 (m, 1H), 7.47-7.38 (m, 2H), |
| | 7.36-7.28 (m, 2H), 4.33-4.12 (m, 3H), 4.09-4.00 (m, 1H), 3.43-3.33 |
| | (m, 4H), 1.99-1.91 (m, 1H), 1.82-1.72 (m, 1H), 1.14-1.04 (m, |
| | 3H). LC-MS (ESI, m/z): [M + H]⁺ 370. |
| 3-15- 04 | 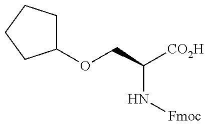 |
| | (2S)-3-(cyclopentyloxy)-2-({[(9H-fluoren-9- |
| | yl)methoxy]carbonyl}amino)propanoic acid |
| | Reference example-3-2-01 |
| | cyclopentanol (CAS: 96-41-3) |
| | ¹H NMR (500 MHz, DMSO-d₆) δ 12.72 (br, 1H), 7.89 (d, J = 7.5 |
| | Hz, 2H), 7.79-7.68 (m, 2H), 7.66-7.53 (m, 1H), 7.46-7.39 (m, 2H), |
| | 7.37-7.28 (m, 2H), 4.32-4.20 (m, 3H), 4.19-4.10 (m, 1H), |
| | 3.94-3.80 (m, 1H), 3.65-3.46 (m, 2H), 1.72-1.38 (m, 8H). |
| | LC-MS (ESI, m/z): [M + H]⁺ 396. |
| 3-15- 05 | 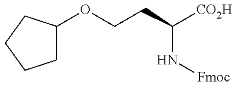 |
| | (2S)-4-(cyclopentyloxy)-2-({[(9H-fluoren-9- |
| | yl)methoxy]carbonyl}amino)butanoic acid |
| | Reference example-3-2-03 |
| | cyclopentanol (CAS: 96-41-3) |
| | ¹H NMR (500 MHz, DMSO-d₆) δ 12.62 (br, 1H), |
| | 7.90 (d, J = 7.6 Hz, 2H), 7.76-7.69 (m, 2H), 7.67-7.51 (m, |
| | 1H), 7.46-7.38 (m, 2H), 7.37-7.27 (m, 2H), 4.36-4.12 (m, |
| | 3H), 4.09-4.00 (m, 1H), 3.90-3.76 (m, 1H), 3.41-3.35 (m, |
| | 2H), 1.97-1.86 (m, 1H), 1.81-1.69 (m, 1H), 1.69-1.37 (m, |
| | 8H). LC-MS (ESI, m/z): [M + H]⁺ 410. |
| 3-15- 06 | 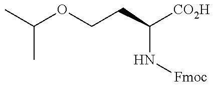 |
| | (2S)-2-({[9H-fluoren-9-yl)methoxy]carbonyl}amino)-4-(propan-2- |
| | yloxy)butanoic acid |
| | Reference example-3-203 |
| | propan-2-ol (CAS: 117072-72-7) |
| | ¹H NMR (500 MHz, DMSO-d₆) δ 12.58 (br, 1H), 7.89 (d, J = 7.5 |
| | Hz, 2H), 7.76-7.61 (m, 2H), 7.55-7.38 (m, 3H), 7.36-7.28 (m, 2H), |
| | 4.35-4.16 (m, 3H), 4.13-3.95 (m, 1H), 3.54-3.44 (m, 1H), |
| | 3.44-3.34 (m, 2H), 1.97-1.86 (m, 1H), 1.80-1.69 (m, 1H), |
| | 1.11-0.99 (m, 6H). LC-MS (ESI, m/z): [M + H]⁺ 384. |
| 3-15- 07 | 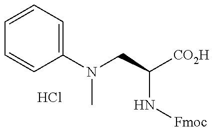 |
| | (2S)-2-({[9H-fluoren-9-yl)methoxy]carbonyl}amino)-3- |
| | [methyl(phenyl)amino]propanoic acid hydrochloride |
| | Reference example-3-2-01 |
| | N-methylaniline (CAS: 100-61-8) |
| | ¹H NMR (300 MHz, DMSO-d₆) δ 12.90 (br, 1H), 8.00-7.77 (m, |
| | 3H), 7.75-7.61 (m, 2H), 7.52-7.11 (m, 7H), 6.84-6.56 (m, 3H), |
| | 4.47-3.99 (m, 4H), 3.92-3.74 (m, 1H), 3.64-3.45 (m, 1H), |
| | 3.01-2.84 (m, 3H). LC-MS (ESI, m/z): [M + H]⁺ 417. |

### Example-3-16-01: (2S)-2-({[(9H-fluoren-9-yl)methoxy]carbonyl}amino)-3-(1,2-oxazol-4-yl)propanoic acid

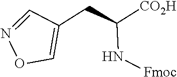

To a solution of (2R)-3,6-dimethoxy-2-(propan-2-yl)-2,5-dihydropyrazine (CAS: 109838-85-9, 4.55 g, 24.7 mmol) in THF (80 mL) at −78° C. was added n-BuLi (19.1 mL, 1.55 M in hexane) dropwise. The mixture was stirred at −78° C. for 30 min. To the reaction mixture at −78° C. was added a solution of 4-(bromomethyl)-1,2-oxazole (CAS: 6455-40-9, 4.00 g, 24.7 mmol) in THF (5.0 mL) dropwise. The resulting mixture was stirred at rt for 3 h. The reaction was then quenched with satd. aq. NH₄Cl. The mixture was extracted with EtOAc. The organic extracts were washed with brine, dried over Na₂SO₄, filtered, and concentrated. The resulting residue was purified by silica gel flash column chromatography (isocratic, hexane/EtOAc=90/10). Appropriate fractions were collected and concentrated. To a solution of the obtained material (5.17 g, 19.5 mmol) in MeOH (50 mL) at 0° C. was added 4M aq. HCl (14.7 mL). The mixture was stirred at rt for 4 h. The mixture was poured into satd. aq. NaHCO₃. The mixture was extracted with EtOAc. The combined organic extracts were washed with brine, dried over Na₂SO₄, filtered, and concentrated. The resulting residue was purified by silica gel flash column chromatography (isocratic, hexane/EtOAc=50/50). Appropriate fractions were collected and concentrated. To a solution of the obtained material (0.80 g, 4.70 mmol) in 1,4-dioxane (30 mL) and H₂O (20 mL) at 0° C. was added NaHCO₃ (1.98 g, 23.5 mmol) and (9H-fluoren-9-yl)methyl carbonochloridate (CAS: 28920-43-6, 1.34 g, 5.17 mmol). The mixture was stirred at rt for 3 h, and then extracted with EtOAc. The organic extracts were washed with brine, dried over Na₂SO₄, filtered, and concentrated. The resulting residue was purified by silica gel flash column chromatography (gradient, hexane/EtOAc=100/0 to 50/50). Appropriate fractions were collected and concentrated. To a solution of the obtained material (1.75 g, 4.46 mmol) in ClCH₂CH₂Cl (20 mL) was added trimethyltin hydroxide (3.23 g, 17.8 mmol). The mixture was stirred at 55° C. for 3 h. The resulting mixture was concentrated. The resulting residue was purified by silica gel flash column chromatography precharged with KF powder (gradient, CH₂Cl₂/MeOH=100/0 to 85/15) to afford the title compound (1.06 g, 2.79 mmol). ¹H NMR (500 MHz, DMSO-d₆) δ 12.64 (br, 1H), 8.72-8.59 (m, 1H), 8.52-8.38 (m, 1H), 7.89 (d, J=7.6 Hz, 2H), 7.78-7.54 (m, 3H), 7.46-7.37 (m, 2H), 7.36-7.27 (m, 2H), 4.33-4.17 (m, 3H), 4.15-4.00 (m, 1H), 2.99-2.90 (m, 1H), 2.81-2.70 (m, 1H). LC-MS (ESI, m/z): [M+H]⁺ 379.

### Example-3-16

The following compounds can be synthesized as outlined for the preparation of Example-3-4-07 or Example-3-16-01 or the methods described in Organic Letters (2019), 21(9), 3178. employing appropriate starting materials in the table.

| | Structure and IUPAC name |
|---|---|
| | Starting material-A |
| Ex- | Starting material-B |
| ample | NMR and MS |
| 3-16- 02 | 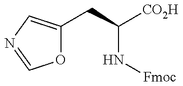 |
| | (2S)-2-({[(9H-fluoren-9-yl)methoxy]carbonyl}amino)-3-(1,3-oxazol- |
| | 5-yl)propanoic acid |
| | 9-chloro-1-[(2S)-1-[3,4-dichlorophenyl)methyl]pyrrolidine-2- |
| | carbonyl]-7-phenyl-1H,4H,5H-benzo[d]1-oxa-3,7-diaza-2- |
| | nickelacyclononan-4-one (CAS: 2089661-84-5) |
| | 5-(bromomethyl)-1,3-oxazole (CAS: 127232-42-2) |
| | ¹H NMR (300 MHz, Methanol-d₄) δ 8.09 (s, 1H), 7.80 (d, J = 7.4 |
| | Hz, 2H), 7.71-7.56 (m, 2H), 7.43-7.35 (m, 2H), 7.35-7.28 (m, 2H), |
| | 6.91 (s, 1H), 4.56-4.44 (m, 1H), 4.42-4.17 (m, 3H), |
| | 3.37-3.34 (m, 1H), 3.21-3.11 (m, 1H). |
| | LC-MS (ESI, m/z): [M + H]⁺ 379. |
| 3-16- 03 | 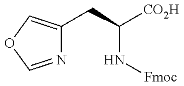 |
| | (2S)-2-({[(9H-fluoren-9-yl)methoxy]carbonyl}amino)-3-(1,3-oxazol- |
| | 4-yl)propanoic acid |
| | 9-chloro-1-[(2S)-1-[(3,4-dichlorophenyl)methyl]pyrrolidine-2- |
| | carbonyl]-7-phenyl-1H,4H,5H-benzo[d]1-oxa-3,7-diaza-2- |
| | nickelacyclonan-4-one (CAS: 2089661-84-5) |
| | 4-(bromomethyl)-1,3-oxazole (CAS: 1065073-37-1) |
| | ¹H NMR (300 MHz, Methanol-d₄) δ 8.13 (s, 1H), 7.79 (d, J = 7.5 |
| | Hz, 2H), 7.70-7.57 (m, 3H), 7.43-7.35 (m, 2H), 7.35-7.25 (m, 2H), |
| | 4.56-4.44 (m, 1H), 4.41-4.28 (m, 2H), 4.27-4.15 (m, 1H), 3.17-3.06 |
| | (m, 1H), 3.06-2.90 (m, 1H). LC-MS (ESI, m/z): [M + H]⁺ 379. |
| 3-16- 04 | 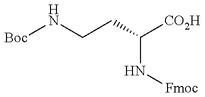 |
| | (2R)-4-{[(tert-butoxy)carbonyl]amino}-2-({[(9H-fluoren-9- |
| | yl)methoxy]carbonyl}amino)butanoic acid |
| | 9-chloro-1-[(2R)-1-[(3,4-dichlorophenyl)methyl]pyrrolidine-2- |
| | carbonyl]-7-phenyl-1H,4H,5H-benzo[d]1-oxa-3,7-diaza-2- |
| | nickelacyclononan-4-one (CAS: 2408623-29-8) |
| | tert-butyl N-(2-bromoethyl)carbamate (CAS: 39684-80-5) |
| | ¹H NMR (500 MHz, DMSO-d₆) δ 12.65 (br, 1H), 7.90 (d, J = 7.5 |
| | Hz, 2H), 7.79-7.60 (m, 3H), 7.50-7.38 (m, 2H), 7.37-7.29 (m, 2H), |
| | 6.99-6.72 (m, 1H), 4.40-4.15 (m, 3H), 4.02-3.93 (m, 1H), |
| | 3.11-2.92 (m, 2H), 1.95-1.82 (m, 1H), 1.77-1.65 (m, 1H), |
| | 1.38 (s, 9H). LC-MS (ESI, m/z): [M − Boc + H]⁺ 341. |
| 3-16- 05 | 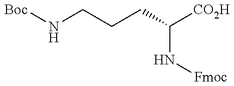 |
| | (2R)-7-{[(tert-butoxy)carbonyl]amino}-2-({[(9H-fluoren-9- |
| | yl)methoxy]carbonyl}amino)heptanoic acid |
| | 9-chloro-1-[(2R)-1-[(3,4-dichlorophenyl)methyl]pyrrolidine-2- |
| | carbonyl]-7-phenyl-1H,4H,5H-benzo[d]1-oxa-3,7-diaza-2- |
| | nickelacyclononan-4-one (CAS: 2408623-29-8) |
| | tert-butyl N-(5-bromopentyl)carbamate (CAS: 83948-54-3) |
| | ¹H NMR (300 MHz, DMSO-d₆) δ 12.55 (br, 1H), 7.89 (d, J = 7.5 |
| | Hz, 2H), 7.78-7.68 (m, 2H), 7.67-7.56 (m, 1H), 7.47-7.37 (m, 2H), |
| | 7.36-7.26 (m, 2H), 6.83-6.69 (m, 1H), 4.36-4.16 (m, 3H), |
| | 3.98-3.84 (m, 1H), 2.96-2.81 (m, 2H), 1.75-1.51 (m, 2H), |
| | 1.44-1.13 (m, 15H). LC-MS (ESI, m/z): [M + H]⁺ 483. |

### Example-3-17

The following compounds can be synthesized as outlined for the preparation of Example-3-3-01, Example-3-5-04, Example-3-10-07, Example-3-13-04, or Reference example-3-4-01 employing appropriate starting materials in the table.

| | Structure and IUPAC name |
|---|---|
| | Starting material-A |
| Ex- | Starting material-B |
| ample | NMR and MS |
| 3-17- 01 | 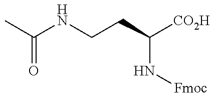 |
| | (2S)-4-acetamido-2-({[(9H-fluoren-9- |
| | yl)methoxy]carbonyl}amino)butanoic acid |
| | (2S)-4-amino-2-({[(9H-fluoren-9-yl)methoxy]carbonyl}amino) |
| | butanoic acid (CAS: 161420-87-7) |
| | acetic anhydride (CAS: 108-24-7) |
| | ¹H NMR (300 MHz, DMSO-d₆) δ 12.63 (br, 1H), 7.99-7.82 (m, |
| | 3H), 7.80-7.58 (m, 3H), 7.51-7.38 (m, 2H), 7.37-7.23 (m, 2H), |
| | 4.36-4.13 (m, 3H), 4.11-3.91 (m, 1H), 3.20-2.99 (m, 2H), |
| | 1.96-1.63 (m, 5H). LC-MS (ESI, m/z): [M + H]⁺ 383 |
| 3-17- 02 | 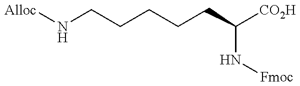 |
| | (2S)-2-({[9H-fluoren-9-yl)methoxy]carbonyl}amino)-7-{[(prop-2-en- |
| | 1-yloxy)carbonyl]amino}heptanoic acid |
| | (2S)-7-amino-2-({[(9H-fluoren-9-yl)methoxy]carbonyl}amino) |
| | heptanoic acid (CAS: 850209-11-9) |
| | allyl chloroformate (CAS: 2937-50-0) |
| | ¹H NMR (400 MHz, DMSO-d₆) δ 12.59 (br, 1H), 7.90 (d, J = 7.5 |
| | Hz, 2H), 7.80-7.69 (m, 2H), 7.61-7.50 (m, 1H), 7.47-7.38 (m, 2H), |
| | 7.37-7.27 (m, 2H), 7.24-7.00 (m, 1H), 6.00-5.81 (m, 1H), 5.33-5.21 |
| | (m, 1H), 5.20-5.09 (m, 1H), 4.54-4.40 (m, 2H), 4.35-4.14 (m, |
| | 3H), 3.99-3.81 (m, 1H), 3.13-2.90 (m, 2H), 1.78-1.55 (m, 2H), |
| | 1.47-1.15 (m, 6H). LC-MS (ESI, m/z): [M + H]⁺ 467. |
| 3-17- 03 | 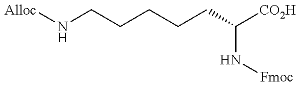 |
| | (2R)-2-({[(9H-fluoren-9-yl)methoxy]carbonyl}amino)-7-{[(prop-2- |
| | en-1-yloxy)carbonyl]amino}heptanoic acid |
| | (2R)-7-amino-2-({[(9H-fluoren-9-yl)methoxy]carbonyl}amino) |
| | heptanoic acid |
| | allyl chloroformate (CAS: 2937-50-0) |
| | Substantially identical for Example 3-17-02 |

### Example-3-18

The following compounds can be synthesized as outlined for the preparation of Example-3-12-02, Example-3-12-04, or Reference example-3-5-01 employing appropriate starting materials in the table.

| | Structure and IUPAC name |
|---|---|
| | Starting material-A |
| Ex- | Starting material-B |
| ample | NMR and MS |
| 3-18- 01 | 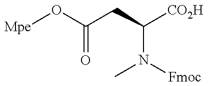 |
| | (2S)-2-({[(9H-fluoren-9-yl)methoxy]carbonyl}(methyl)amino)-4-[(3- |
| | methylpentan-3-yl)oxy]-4-oxobutanoic acid |
| | (3S)-3-({[(9H-fluoren-9-yl)methoxy]carbonyl}(methyl)amino)-4- |
| | oxo-4-(prop-2-en-1-yloxy)butanoic acid (CAS: 1608089-69-5) |
| | 3-methylpentan-3-ol (CAS: 77-74-7) |
| | ¹H NMR (500 MHz, DMSO-d₆) δ 13.11 (br, 1H), 7.93-7.86 (m, |
| | 2H), 7.69-7.61 (m, 2H), 7.46-7.39 (m, 2H), 7.39-7.27 (m, 2H), |
| | 4.97-4.72 (m, 1H), 4.39-4.13 (m, 3H), 2.91-2.75 (m, 4H), 2.70- |
| | 2.57 (m, 1H), 1.88-1.59 (m, 4H), 1.35-1.21 (m, 3H), 0.86- |
| | 0.70 (m, 6H). LC-MS (ESI, m/z): [M − Mpe + H]⁺ 370. |
| 3-18- 02 | 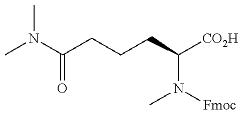 |
| | (2S)-5-(dimethylcarbamoyl)-2-({[(9H-fluoren-9- |
| | yl)methoxy]carbonyl}(methyl)amino)pentanoic acid |
| | (2S)-6-(tert-butoxy)-2-({[(9H-fluoren-9- |
| | yl)methoxy]carbonyl}(methyl)amino)-6-oxohexanoic acid (CAS: |
| | 2382599-28-0) |
| | dimethylamine (CAS: 124-40-3) |
| | ¹H NMR (300 MHz, DMSO-d₆) δ 12.83 (br, 1H), 8.01-7.80 (m, |
| | 2H), 7.75-7.55 (m, 2H), 7.51-7.21 (m, 4H), 4.64-4.16 (m, 4H), |
| | 2.93 (s, 3H), 2.81 (s, 3H), 2.79-2.72 (m, 3H), 2.40-2.19 (m, 2H), |
| | 1.93-1.57 (m, 2H), 1.54-1.29 (m, 2H). |
| | LC-MS (ESI, m/z): [M + H]⁺ 425. |

### Example-3-19

The following compounds can be synthesized as outlined for the preparation of Example-3-1-02. Example-3-1-04, or Example-3-1-08 employing appropriate starting materials in the table.

| | Structure and IUPAC name |
|---|---|
| Ex- | Starting material-A |
| am- | Starting material-B |
| ple | NMR and MS |
| 3-19- 01 | 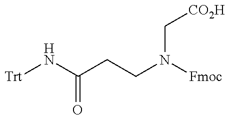 |
| | 2-({[(9H-fluoren-9-yl)methoxy]carbonyl}({2- |
| | [(triphenylmethyl)carbamoyl]ethyl})amino)acetic acid |
| | 3-Amino-N-(triphenylmethyl)propanamide (CAS: 1203712-73-5) |
| | 2-oxoacetic acid hydrate (CAS: 1152495-30-1) |
| | ¹H NMR (500 MHz, DMSO-d₆) δ 12.93-12.46 (m, 1H), |
| | 8.79-8.60 (m, 1H), 7.93-7.85 (m, 2H), 7.69-7.58 (m, 2H), |
| | 7.45-7.37 (m, 2H), 7.34-7.12 (m, 17H), 4.32-4.19 (m, 3H), |
| | 3.89-3.77 (m, 2H), 3.49-3.56 (m, 2H), 2.58-2.52 (m, 2H). |
| | LC-MS (ESI, m/z): [M + H]⁺ 611. |

### Example-3-20

The following compounds can be synthesized as outlined for the preparation of Reference example-3-6-01, Example-3-1-01, Example-3-4-01, or Example-3-4-02 employing appropriate starting materials in the table.

| | Structure and IUPAC name |
|---|---|
| Ex- | Starting material-A |
| ample | NMR and MS |
| 3-20- 01 | 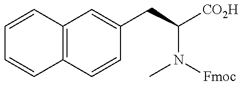 |
| | (2S)-2-({[(9H-fluoren-9-yl)methoxy]carbonyl}(methyl)amino)-3- |
| | (naphthalen-2-yl)propanoic acid |
| | (2S)-2-({[(9H-fluoren-9-yl)methoxy]carbonyl}amino)-3-(naphthalen- |
| | 2-yl)propanoic acid (CAS: 112883-43-9) |
| | ¹H NMR (400 MHz, DMSO-d₆) δ 13.81-12.72 (m, 1H), |
| | 7.89-7.73 (m, 5H), 7.66-7.09 (m, 10H), 4.98-4.76 (m, 1H), |
| | 4.33-4.05 (m, 3H), 3.51-3.21 (m, 2H), 2.80-2.69 (m, 3H). |
| | LC-MS (ESI, m/z): [M + H]⁺ 452. |
| 3-20- 02 | 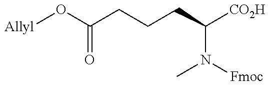 |
| | (2S)-2-({[(9H-fluoren-9-yl)methoxy]carbonyl}(methyl)amino)- |
| | 6-oxo-6-(prop-2-en-1-yloxy)hexanoic acid |
| | (2S)-2-({[(9H-fluoren-9-yl)methoxy]carbonyl}amino)-6-oxo- |
| | 6-(prop-2-en-1-yloxy)hexanoic acid (CAS: 133464-45-6) |
| | ¹H NMR (500 MHz, DMSO-d₆) δ 12.82 (br, 1H), 7.95-7.80 (m, |
| | 2H), 7.70-7.54 (m, 2H), 7.47-7.37 (m, 2H), 7.36-7.23 (m, 2H), |
| | 6.01-5.82 (m, 1H), 5.36-5.25 (m, 1H), 5.22-5.13 (m, 1H), 4.62- |
| | 4.23 (m, 6H), 2.80-2.65 (m, 3H), 2.45-2.26 (m, 2H), 1.90- |
| | 1.54 (m, 2H), 1.51-1.25 (m, 2H). LC-MS (ESI, m/z): |
| | [M + H]⁺ 438. |
| 3-20- 03 | 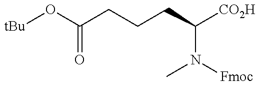 |
| | (2S)-6-(tert-butoxy)-2-({[(9H-fluoren-9- |
| | yl)methoxy]carbonyl}(methyl)amino)-6-oxohexanoic acid |
| | (2S)-2-{[(benzyloxy)carbonyl]amino}-6-(tert-butoxy)-6-oxohexanoic |
| | acid (CAS: 201668-29-3) |
| | ¹H NMR (400 MHz, DMSO-d₆) δ 12.96 (br, 1H), 7.99-7.83 (m, |
| | 2H), 7.71-7.57 (m, 2H), 7.48-7.38 (m, 2H), 7.38-7.24 (m, |
| | 2H), 4.57-4.18 (m, 4H), 2.80-2.64 (m, 3H), 2.31-2.11 (m, 2H), |
| | 1.89-1.54 (m, 2H), 1.48-1.26 (m, 11H). LC-MS (ESI, m/z): |
| | [M + H]⁺ 454. |

### Example-3-21-01: (2S,4R)-4-(tert-butoxy)-1-{[(9H-fluoren-9-yl)methoxy]carbonyl}pyrrolidine-2-carboxylic acid

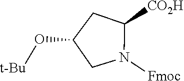

The title compound can be synthesized as outlined for the preparation methods described in WO2014/074658 A1. employing appropriate starting material 1-tert-butyl 2-methyl (2S,4R)-4-hydroxypyrrolidine-1,2-dicarboxylate (CAS: 74844-91-0) and 2-Bromo-2-methylpropane (CAS: 507-19-7). ¹H NMR (500 MHz, DMSO-d₆) δ 12.7 (br, 1H), 7.92-7.87 (m, 2H), 7.68-7.63 (m, 2H), 7.42 (dd, J=7.5 Hz, 2H), 7.33 (dd, J=7.5 Hz, 2H), 4.40-4.10 (m, 5H), 3.61-3.12 (m, 2H), 2.24-1.95 (m, 2H), 1.17-1.09 (m, 9H). LC-MS (ESI, m/z): [M+H]⁺ 410.

### Example-3-21-02: (2R,4S)-4-{[(tert-butoxy)carbonyl]amino}-1-{[(9H-fluoren-9-yl)methoxy]carbonyl}pyrrolidine-2-carboxylic acid

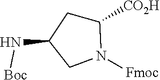

The title compound can be synthesized as outlined for the preparation methods described in Amino Acids (2014), 46(11), 2517. employing appropriate starting material 1,2-dibenzyl (2R,4R)-4-hydroxypyrrolidine-1,2-dicarboxylate (CAS: 142776-07-6). ¹H NMR (500 MHz, DMSO-d₆) δ 12.7 (br, 1H), 7.90 (dd, J=7.0 Hz, 2H), 7.68-7.55 (m, 2H), 7.45-7.38 (m, 2H), 7.38-7.25 (m, 3H), 4.50-4.12 (m, 5H), 3.62-3.51 (m, 1H), 3.51-3.12 (m, 1H), 2.21-1.95 (m, 2H), 1.40 (s, 9H). LC-MS (ESI, m/z): [M+H]⁺ 453.

### Example-3-21-03: (2R,4R)-4-{[(tert-butoxy)carbonyl]amino}-1-{[(9H-fluoren-9-yl)methoxy]carbonyl}pyrrolidine-2-carboxylic acid

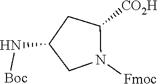

The title compound can be synthesized as outlined for the preparation methods described in Amino Acids (2014), 46(11), 2517. employing appropriate starting material 1,2-dibenzyl (2R,4S)-4-hydroxypyrrolidine-1,2-dicarboxylate (CAS: 1190194-20-7). ¹H NMR (500 MHz, DMSO-d₆) δ 12.81 (br, 1H), 7.95-7.85 (m, 2H), 7.70-7.61 (m, 2H), 7.47-7.38 (m, 2H), 7.38-7.28 (m, 2H), 7.09-6.96 (m, 1H), 4.38-4.08 (m, 4H), 4.08-3.90 (m, 1H), 3.80-3.62 (m, 1H), 3.19-3.02 (m, 1H), 2.61-2.39 (m, 1H), 1.91-1.69 (m, 1H), 1.39 (s, 9H). LC-MS (ESI, m/z): [M+H−Boc]⁺ 353.

### Example-3-22

The following compounds can be synthesized as outlined for the preparation of Example-3-8-05 employing appropriate starting materials in the table.

| Ex- | Structure and IUPAC name |
|---|---|
| am- | Starting material-A |
| ple | NMR and MS |
| 3-22- 01 | 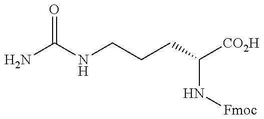 |
| | (2R)-5-(carbamoylamino)-2-({[(9H-fluoren-9- |
| | yl)methoxy]carbonyl}amino)pentanoic acid |
| | (2R)-5-{[(tert-butoxy)carbonyl]amino}-2-({[9H-fluoren-9- |
| | yl)methoxy]carbonyl}amino)pentanoic acid (CAS: 118476-89-4) |
| | ¹H NMR (500 MHz, DMSO-d₆) δ 12.61 (br, 1H), 7.90 (d, J = 7.5 |
| | Hz, 2H), 7.79-7.60 (m, 3H), 7.48-7.39 (m, 2H), 7.36-7.29 (m, 2H), |
| | 6.06-5.88 (m, 1H), 5.40 (br, 2H), 4.33-4.15 (m, 3H), 4.00-3.87 (m, |
| | 1H), 3.03-2.86 (m, 2H), 1.78-1.63 (m, 1H), 1.62-1.51 (m, 1H), |
| | 1.50-1.32 (m, 2H). LC-MS (ESI, m/z): [M + H]⁺ 398. |
| 3-22- 02 | 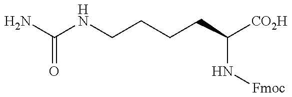 |
| | (2S)-6-(carbamoylamino)-2-({[(9H-fluoren-9- |
| | yl)methoxy]carbonyl}amino)hexanoic acid |
| | (2S)-6-{[(tert-butoxy)carbonyl]amino}-2-({[(9H-fluoren-9- |
| | yl)methoxy]carbonyl}amino)hexanoic acid (CAS: 71989-26-9) |
| | ¹H NMR (500 MHz, DMSO-d₆) δ 12.59 (br, 1H), 7.90 (d, J = 7.5 |
| | Hz, 2H), 7.74 (d, J = 7.3 Hz, 2H), 7.65 (d, J = 7.93 Hz, 1H), 7.49- |
| | 7.39 (m, 2H), 7.37-7.26 (m, 2H), 5.98-5.84 (m, 1H), 5.37 (br, 2H), |
| | 4.34-4.16 (m, 3H), 3.97-3.84 (m, 1H), 3.05-2.82 (m, 2H), 1.78-1.47 |
| | (m, 2H), 1.43-1.21 (m, 4H). LC-MS (ESI, m/z): [M + H]⁺ 412. |
| 3-22- 03 | 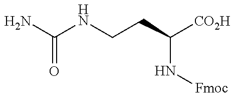 |
| | (2S)-4-(carbamoylamino)-2-({[(9H-fluoren-9- |
| | yl)methoxy]carbonyl}amino)butanoic acid |
| | (2S)-4-{[(tert-butoxy)carbonyl]amino}-2-({[(9H-fluoren-9- |
| | yl)methoxy]carbonyl}amino)butanoic acid (CAS: 125238-99-5) |
| | ¹H NMR (300 MHz, DMSO-d₆) δ 12.68 (br, 1H), 7.89 (d, J = |
| | 7.3 Hz, 2H), 7.80-7.58 (m, 3H), 7.51-7.22 (m, 4H), 6.11-5.91 (m, |
| | 1H), 5.46 (s, 2H), 4.39-4.31 (m, 3H), 4.00-3.88 (m, 1H), |
| | 3.15-2.88 (m, 2H), 1.92-1.76 (m, 1H), 1.74-1.58 (m, 1H). |
| | LC-MS (ESI, m/z): [M + H]⁺ 384. |

### Example-3-23

The following compounds can be synthesized as outlined for the preparation of Reference example-3-1-04, Example-3-2-06, Example-3-3-05, or Example-3-7-04 employing appropriate starting materials in the table.

| | Structure and IUPAC name |
|---|---|
| Ex- | Starting material-A |
| am- | Starting material-B |
| ple | NMR and MS |
| 3- 23- 01 | 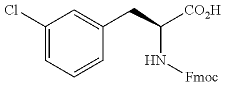 |
| | (2S)-3-(3-chlorophenyl)-2-({[(9H-fluoren-9- |
| | yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | 1-bromo-3-chlorobenzene (CAS: 108-37-2) |
| | ¹H NMR (300 MHz, DMSO-d₆) d 7.86 (d, J = 7.4 Hz, 2H), |
| | 7.77-7.17 (m, 11H), 4.27 (m, 4H), 3.09 (dd, J = |
| | 14.0, 4.3 Hz, 1H), 2.86 (dd, J = |
| | 13.7, 10.9 Hz, 1H). LC-MS (ESI, m/z): [M − H⁻] 420. |
| 3- 23- 02 | 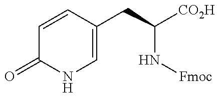 |
| | (2S)-2-({[(9H-fluoren-9-yl)methoxy]carbonyl}amino)-3-(6-oxo-1,6- |
| | dihydropyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromo-2-(methoxymethoxy)pyridine (CAS: 247189-74-8) |
| | ¹H NMR (400 MHz, DMSO-d₆) δ 11.88 (br, 1H), 7.89 (d, J = 7.5 |
| | Hz, 2H), 7.79-7.50 (m, 3H), 7.48-7.37 (m, 3H), 7.36-7.20 (m, 3H), |
| | 6.39-6.25 (m, 1H), 4.32-4.00 (m, 4H), 2.91-2.74 (m, 1H), |
| | 2.69-2.53 (m, 1H). LC-MS (ESI, m/z): [M + H]⁺ 405. |
| 3- 23- 03 | 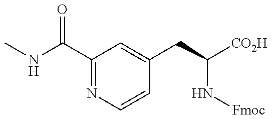 |
| | (2S)-2-({[(9H-fluoren-9-yl)methoxy]carbonyl}amino)-3-[2- |
| | (methylcarbamoyl)pyridin-3-yl]propanoic acid |
| | methyl (2R)-2-({[(9H-fluoren-9-yl)methoxy]carbonyl}amino)-3- |
| | iodopropanoate (CAS: 156017-42-4) |
| | 4-bromo-N-methylpyridine-2-carboxamide (CAS: 1209459-88-0) |
| | ¹H NMR (500 MHz, DMSO-d₆) δ 12.95 (br, 1H), 8.73 (d, J = 5.0 |
| | Hz, 1H), 8.48 (d, J = 4.8 Hz, 1H), 8.09-7.83 (m, 3H), 7.76-7.22 (m, |
| | 8H), 4.39-4.01 (m, 4H), 3.25-3.11 (m, 1H), 3.06-2.91 (m, 1H), |
| | 2.80 (d, J = 4.8 Hz, 3H). LC-MS (ESI, m/z): [M + H]⁺ 447. |
| 3- 23- 04 | 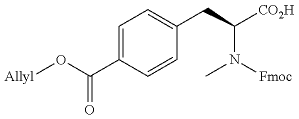 |
| | (2S)-2-({[(9H-fluoren-9-yl)methoxy]carbonyl}(methyl)amino)-3-{4- |
| | [(prop-2-en-1-yloxy)carbonyl]phenyl}propanoic acid |
| | Reference example 3-1-01 |
| | tert-butyl 4-bromobenzoate (CAS: 59247-47-1) |
| | ¹H NMR (500 MHz, CDCl₃) d 8.00-7.85 (m, 2H), 7.78-7.67 (m, |
| | 2H), 7.53-6.90 (m, 8H), 6.08-5.96 (m, 1H), 5.44-5.35 (m, 1H), |
| | 5.32-5.23 (m, 1H), 4.85-4.76 (m, 2H), 4.74-4.57 (m, 1H), |
| | 4.50-4.30 (m, 2H), 4.21-4.14 (m, 1H), 3.43-3.30 (m, 1H), |
| | 3.24-3.05 (m, 1H), 2.70 (s, 3H). LC-MS (ESI, m/z): |
| | [M + H]⁺ 486. |
| 3- 23- 05 | 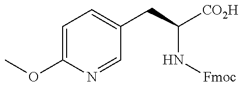 |
| | (2S)-2-({[(9H-fluoren-9-yl)methoxy]carbonyl}amino)-3-(6- |
| | methoxypyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromo-2-methoxypyridine (CAS: 13472-85-0) |
| | ¹H NMR (300 MHz, DMSO-d₆) δ 12.76 (br, 1H), 8.07-7.98 (m, |
| | 1H), 7.88 (d, J = 7.4 Hz, 2H), 7.77-7.56 (m, 4H), 7.45-7.24 (m, |
| | 4H), 6.72 (d, J = 8.4 Hz, 1H), 4.24-4.08 (m, 4H), |
| | 3.80 (s, 3H), 3.08-2.95 (m, 1H), 2.87-2.69 (m, 1H). |
| | LC-MS (ESI, m/z): [M + H]⁺ 419. |
| 3- 23- 06 | 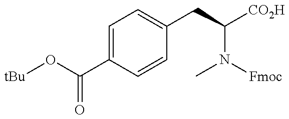 |
| | (2S)-3-{4-[(tert-btuoxy)carbonyl]phenyl}-2-({[(9H-fluoren-9- |
| | yl)methoxy]carbonyl}(methyl)amino)propanoic acid |
| | Reference example-3-1-01 |
| | tert-butyl 4-bromobenzoate (CAS: 59247-47-1) |
| | ¹H NMR (400 MHz, DMSO-d₆) δ 12.97 (br, 1H), 7.97-7.84 (m, |
| | 2H), 7.82-7.66 (m, 2H), 7.63-7.09 (m, 8H), 4.89-4.63 (m, |
| | 1H), 4.37-4.10 (m, 3H), 3.32-2.87 (m, 2H), 2.74-2.61 (m, 3H), |
| | 1.63-1.43 (m, 9H). LC-MS (ESI, m/z): [M − tBu + H]⁺ 446. |
| 3- 23- 07 | 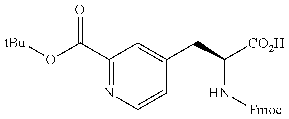 |
| | (2S)-3-{5-[(tert-butoxy)carbonyl]pyridin-3-yl}-2-({[(9H-fluoren-9- |
| | yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | tert-butyl 5-bromopyridine-3-carboxylate (CAS: 263270-02-06) |
| | ¹H NMR (500 MHz, DMSO-d₆) δ 13.00 (br, 1H), 8.93-8.84 (m, |
| | 1H), 8.74-8.62 (m, 1H), 8.17 (s, 1H), 7.87 (d, J = 7.6 Hz, 2H), |
| | 7.63 (br, 1), 7.63-7.44(m, 2H), 7.44-7.34 (m, 2H), 7.33-7.19 (m, |
| | 2H), 4.31-4.07 (m, 4H), 3.26-3.06 (m, 1H), 3.04-2.81 (m, |
| | 1H), 1.56-1.46 (m, 9H). LC-MS (ESI, m/z): [M + H]⁺ 490. |
| 3- 23- 08 | 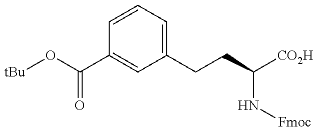 |
| | (2S)-4-{3-[(tert-butoxy)carbonyl]phenyl}-2-({[(9H-fluoren-9- |
| | yl)methoxy]carbonyl}amino)butanoic acid |
| | Reference example-3-1-03 |
| | tert-butyl 3-bromobenzoate (CAS: 69038-74-0) |
| | ¹H NMR (300 MHz, DMSO-d₆) δ 12.65 (br, 1H), 7.90 (d, J = 9.6 |
| | Hz, 2H), 7.82-7.71 (m, 5H), 7.51-7.28 (m, 6H), 4.38-4.12 (m, |
| | 3H), 4.07-3.77 (m, 1H), 2.82-2.58 (m, 2H), 2.15-1.75 (m, 2H), |
| | 1.53 (s, 9H). LC-MS (ESI, m/z): [M − tBu + H ]⁺ 446. |
| 3- 23- 09 | 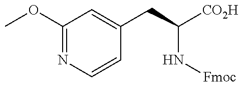 |
| | (2S)-2-({[(9H-fluoren-9-yl)methoxy]carbonyl}amino)-3-(2- |
| | methoxypyridin-4-yl)propanoic acid |
| | methyl (2R)-2-({[(9H-fluoren-9-yl)methoxy]carobnyl}amino)-3- |
| | iodopropanoate (CAS: 156017-42-4) |
| | 4-bromo-2-methoxypyridine (CAS: 100367-39-3) |
| | ¹H NMR (300 MHz, DMSO-d₆) δ 12.86 (s, 1H), 8.05 (d, J = 5.1 |
| | Hz, 1H), 7.98-7.72 (m, 3H), 7.72-7.20 (m, 6H), 6.90 (d, J = 5.4 Hz, |
| | 1H), 6.74 (s,1H), 4.30-4.12 (m, 4H), 3.81 (s, 3H), 3.19-2.95 (m, |
| | 1H), 2.95-2.79 (m, 1H). LC-MS (ESI, m/z): [M + H]⁺ 419. |
| 3- 23- 10 | 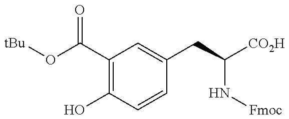 |
| | (2S)-3-{3-[(tert-butoxy)carbonyl]-4-hydroxyphenyl}-2-({[(9H- |
| | fluoren-9-yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | tert-butyl 5-bromo-2-hydroxybenzoate (CAS: 224775-76-2) |
| | ¹H NMR (300 MHz, DMSO-d₆) δ 12.89 (s, 1H), 10.64 (s, 1H), |
| | 7.88 (d, J = 7.5 Hz, 2H), 7.83-7.11 (m, 9H), 6.87 (d, J = 8.4 Hz, |
| | 1H), 4.38-3.97 (m, 4H), 3.19-2.93 (m, 1H), 2.93-2.66 (m, 1H), |
| | 1.54 (s, 9H). LC-MS (ESI, m/z): [M − H]⁺ 602. |
| 3- 23- 11 | 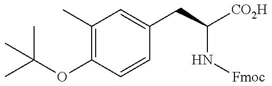 |
| | (2S)-3-[4-(tert-butoxy)-3-methylphenyl]-2-({[(9H-fluoren-9- |
| | yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | 4-bromo-1-(tert-butoxy)-2-methylbenzene (CAS: 1309933-94-5) |
| | ¹H NMR (300 MHz, DMSO-d₆) δ 12.70 (s, 1H), 7.88 (d, J = 7.5 |
| | Hz, 2H), 7.80-7.48 (m, 3H), 7.48-7.15 (m, 4H), 7.15-6.81 (m, |
| | 3H), 4.30-3.97 (m, 4H), 3.13-2.99 (m, 1H), 2.99-2.64 (m, 1H), |
| | 2.10 (s, 3H), 1.26 (s, 9H). LC-MS (ESI, m/z): [M + H]⁺ 474. |
| 3-23- 12 | 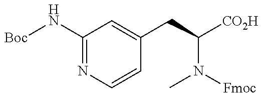 |
| | (2S)-3-(2-{[(tert-butoxy)carbonyl]amino}pyridin-4-yl)-2-({[(9H- |
| | fluoren-9-yl)methoxy]carbonyl}(methyl)amino)propanoic acid |
| | Reference example-3-1-01 |
| | tert-butyl N-(4-bromopyridin-2-yl)carbamate (CAS: 207799-10-8) |
| | ¹H NMR (400 MHz, DMSO-d₆) δ 13.03 (s, 1H), 9.71 (d, J = 15.2 |
| | Hz, 1H), 8.17-8.03 (m, 1H), 7.88 (d, J = 7.6 Hz, 2H), 7.84-7.24 (m, |
| | 7H), 6.92-6.60 (m, 1H), 4.89-4.75 (m, 1H), 4.34-4.10 (m, 3H), |
| | 3.26-2.83 (m, 2H), 2.83-2.67 (m, 3H), 1.45-1.36 (m, 9H). |
| | LC-MS (ESI, m/z): [M + H]⁺ 518. |
| 3- 23- 13 | 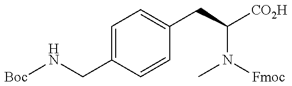 |
| | (2S)-3-[4-({[(tert-butoxy)carbonyl]amino}methyl)phenyl]-2-({[(9H- |
| | fluoren-9-yl)methoxy]carbonyl}(methyl)amino)propanoic acid |
| | Reference example-3-1-01 |
| | tert-butyl N-[(4-bromophenyl)methyl]carbamate (CAS: 68819-84-1) |
| | ¹H NMR (400 MHz, DMSO-d₆) δ12.97 (br, 1H), 7.90 (d, J = |
| | 7.2 Hz, 2H), 7.62-7.47 (m, 2H), 7.47-7.26 (m, 5H), 7.26-7.05 (m, |
| | 3H), 6.98 (d, J = 8.0 Hz, 1H), 4.81-4.61 (m, 1H), 4.40-4.14 (m, |
| | 3H), 4.14-3.95 (m, 2H), 3.44-2.95 (m, 2H), 2.88-2.60 (m, |
| | 3H), 1.39 (m, 9H). LC-MS (ESI, m/z): [M − H]⁺ 529. |
| 3- 23- 14 | 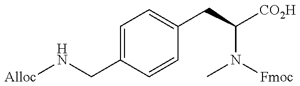 |
| | (2S)-2-({[(9H-fluoren-9-yl)methoxy]carbonyl}(methyl)amino)-3-[4- |
| | ({[(prop-2-en-1-yloxy)carbonyl]amino}methyl)phenyl]propanoic |
| | acid |
| | Reference example-3-1-01 |
| | tert-butyl N-[(4-bromophenyl)methyl]carbamate (CAS: 68819-84-1) |
| | ¹H NMR (300 MHz, DMSO-d₆) δ 12.90 (s, 1H), 7.89 (d, J = 7.5 |
| | Hz, 2H), 7.80-7.65 (m, 1H), 7.65-6.85 (m, 10H), 6.00-5.79 (m, |
| | 1H), 5.36-5.02 (m, 2H), 4.81-4.59 (m, 1H), 4.59-4.41 (m, 2H), |
| | 4.41-4.06 (m, 5H), 3.26-2.75 (m, 2H), 2.75-2.60 (m, |
| | 3H). LC-MS (ESI, m/z): [M + H]⁺ 515. |
| 3- 23- 15 | 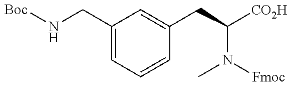 |
| | (2S)-3-[3-({[(tert-butoxy)carbonyl]amino}methyl)phenyl]-2-({[(9H- |
| | fluoren-9-yl)methoxy]carbonyl}(methyl)amino)propanoic acid |
| | Reference example-3-1-01 |
| | tert-butyl N-[(3-bromophenyl)methyl]carbamate |
| | (CAS: 171663-13-1) |
| | ¹H NMR (500 MHz, DMSO-d₆) δ 12.95 (br, 1H), 7.89 (d, J = 7.5 |
| | Hz, 2H), 7.61-7.48 (m, 2H), 7.48-6.87 (m, 9H), 4.81-4.69 (m, |
| | 1H), 4.37-4.14 (m, 3H), 4.14-3.92 (m, 3H), 3.28-2.80 (m, 2H), |
| | 2.77-2.62 (m, 3H), 1.55-1.24 (m, 9H). |
| | LC-MS (ESI, m/z): [M − Boc + H]⁺ 431. |
| 3- 23- 16 | 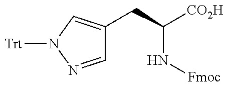 |
| | (2S)-2-({[(9H-fluoren-9-yl)methoxy]carbonyl}amino)-3-[1- |
| | (triphenylmethyl)-1H-pryazol-4-yl]propanoic acid |
| | methyl (2R)-2-({[(9H-fluoren-9-yl)methoxy]carbonyl}amino)-3- |
| | iodopropanoate (CAS: 156017-42-4) |
| | 4-bromo-1-(triphenylmethyl)-1H-pyrazole (CAS: 95162-14-4) |
| | ¹H NMR (500 MHz, DMSO-d₆) δ 12.69 (br, 1H), 7.89 (d, J = |
| | 7.6 Hz, 2H), 7.74-7.62 (m, 3H), 7.52 (s, 1H), 7.45-7.36 (m, 2H), |
| | 7.33-7.24 (m, 12H). 7.03-6.98 (m, 6H), 4.23-4.04 (m, 4H), |
| | 2.94-2.86 (m, 1H), 2.75-2.66 (m, 1H). |
| | LC-MS (ESI, m/z): not found. |
| 3- 23- 17 | 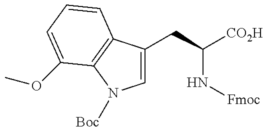 |
| | (2S)-3-{1-[(tert-butoxy)carbonyl]-7-methoxy-1H-indol-3-yl}-2- |
| | ({[(9H-fluoren-9-yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-7-methoxy-1H-indole-1-carboxylate (CAS: |
| | 1823521-06-7) |
| | ¹H NMR (300 MHz, DMSO-d₆) δ 12.82 (br, 1H), 7.90 (d, J = |
| | 7.6 Hz, 2H), 7.73-7.62 (m, 2H), 7.51-7.37 (m, 3H), 7.35-7.17 |
| | (m, 4H), 6.95-6.90 (m, 1H), 4.33-4.13 (m, 4H), 3.89 (s, 3H), |
| | 3.26-3.12 (m, 2H), 3.06-2.94 (m, 1H), 1.55 (s, 9H). |
| | LC-MS (ESI, m/z): [M + Na]⁺ 579. |
| 3- 23- 18 | 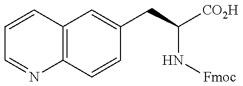 |
| | (2S)-2-({[(9H-fluoren-9-yl)methoxy]carbonyl}amino)-3-(quinolin-6- |
| | yl)propanoic acid |
| | methyl (2R)-2-({[(9H-fluoren-9-yl)methoxy]carbonyl}amino)-3- |
| | idopropanoate (CAS: 156017-42-4) |
| | 6-bromoquinoline (CAS: 5332-25-2) |
| | ¹H NMR (300 MHz, DMSO-d₆) δ 12.82 (br, 1H), 8.89-8.85 (m, |
| | 1H), 8.32-8.26 (m, 1H), 7.98-7.93 (m, 1H), 7.90-7.82 (m, 4H), |
| | 7.76-7.67 (m, 1H), 7.64-7.49 (m, 3H), 7.41-7.34 (m, 2H), |
| | 7.30-7.15 (m, 2H), 4.38-4.29 (m, 1H), 4.22-4.10 (m, |
| | 3H), 3.37-3.26 (m, 1H), 3.20-3.05 (m, 1H). |
| | LC-MS (ESI, m/z): [M + H]⁺ 439. |
| 3- 23- 19 | 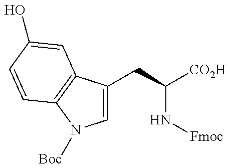 |
| | (2S)-3-{1-[(tert-butoxy)carbonyl]-5-hydroxy-1H-indol-3-yl}-2- |
| | ({[(9H-fluoren-9-yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-4204) |
| | tert-butyl 3-bromo-5-hydroxy-1H-indole-1-carboxylate |
| | ¹H NMR (400 MHz, DMSO-d₆) δ 12.89 (m, 1H), 9.26 (br, 1H), |
| | 7.92-7.59 (m, 5H), 7.54-7.19 (m, 5H), 7.03-6.74 (m, 2H), 4.32- |
| | 4.15 (m, 4H), 3.44-.24 (m, 1H), 3.18-3.07 (m, 1H), 3.02-2.87 (m, |
| | 1H), 1.75-1.46 (m, 9H). LC-MS (ESI, m/z): [M − Boc + H]⁺ 443. |
| 3-23- 20 | 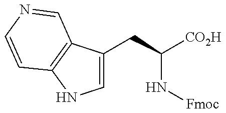 |
| | (2S)-2-({[(9H-fluoren-9-yl)methoxy]carbonyl}amino)-3-{1H- |
| | pyrrolo[3,2-c]pyridin-3-yl}propanoic acid |
| | methyl (2R)-2-({[(9H-fluoren-9-yl)methoxy]carbonyl}amino)-3- |
| | iodopropanoate (CAS: 156017-42-4) |
| | 3-bromo-1H-pyrrolo[3,2-c]pyridne (CAS: 23612-36-4) |
| | ¹H NMR (300 MHz, DMSO-d₆) δ 11.86 (m, 1H), 9.08 (s, 1H), |
| | 8.22 (d, J = 5.9 Hz, 1H), 7.95-7.81 (m, 2H), 7.69-7.52 (m, 4H), |
| | 7.46-7.23 (m, 5H), 4.40-4.09 (m, 5H), 3.37-3.09 (m, 2H). |
| | LC-MS (ESI, m/z): [M + H]⁺ 428. |
| 3- 23- 21 | 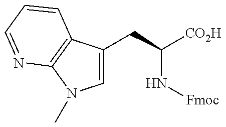 |
| | (2S)-2-({[(9H-fluoren-9-yl)methoxy]carbonyl}amio)-3-{1-methyl- |
| | 1H-pyrrolo[2,3-b]pyridin-3-yl}propanoic acid |
| | methyl (2R)-2-({[9H-fluoren-9-yl)methoxy]carbonyl}amino)-3- |
| | iodopropanoate (CAS: 156017-42-4) |
| | 3-bromo-1-methyl-1H-pyrrolo[2,3-b]pyridine (CAS: 281192-91-4) |
| | ¹H NMR (400 MHz, DMSO-d₆) δ 12.67 (br, 1H), 8.26 (d, J = |
| | 4.6 Hz, 1H), 8.14-8.01 (m, 1H), 7.88 (d, J = 7.6 Hz, 2H), |
| | 7.76 (d, J = 8.4 Hz, 1H), 7.71-7.59 (m, 2H), 7.45-7.37 (m, 2H), |
| | 7.37-7.24 (m, 3H), 7.11-7.05 (m, 1H), 4.31-4.08 (m, 4H), |
| | 3.79-3.71 (m, 3H), 3.26-3.13 (m, 1H), 3.12-2.97 (m, 1H). |
| | LC-MS (ESI, m/z): [M + H]⁺ 442. |
| 3- 23- 22 | 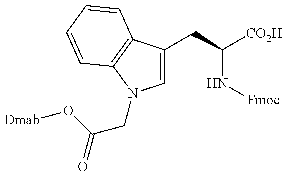 |
| | (2S)-3-(1-{2-[(4-{[1-(4,4-dimethyl-2,6-dioxocyclohexylidene)-3- |
| | methylbutyl]amino}phenyl)methoxy]-2-oxoethyl}-1H-indol-3-yl)-2- |
| | ({[9H-fluoren-9-yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | (4-{[1-(4,4-dimethyl-2,6-dioxocyclohexylidene)-3- |
| | methylbutyl]amino}phenyl)methyl 2-(3-bromo-1H-indol-1-yl)acetate |
| | ¹H NMR (500 MHz, DMSO-d₆) δ 15.27 (s, 1H), 12.90-12.68 (m, |
| | 1H), 7.91-7.83 (m, 2H), 7.72-7.63 (m, 2H), 7.63-7.57 (m, 1H), |
| | 7.45-7.37 (m, 4H), 7.35-7.26 (m, 4H), 7.19 (s, 1H), 7.15-7.07 (m, |
| | 1H), 7.06-7.02 (m, 1H), 5.22-5.11 (m, 4H), 4.23-4.15 (m, 3H), |
| | 3.24-3.15 (m, 1H), 3.05-2.94 (m, 2H), 2.38 (br, 3H), 1.75-1.65 (m, |
| | 1H), 1.29-1.22 (m, 3H), 1.00 (s, 6H), 0.90-0.81 (m, 2H), |
| | 0.72-0.64 (m, 6H). LC-MS (ESI, m/z): [M + H]⁺ 797. |
| 3- 23- 23 | 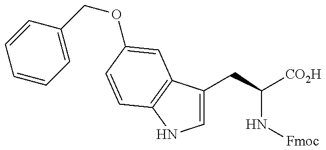 |
| | (2S)-3-[5-(benzyloxy)-1H-indol-3-yl]-2-({[(9H-fluoren-9- |
| | yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | 5-(benzyloxy)-3-bromo-1H-indole (CAS: 569337-39-9) |
| | ¹H NMR (400 MHz, DMSO-d₆) δ 12.69 (br, 1H), 10.78-10.67 (m, |
| | 1H), 7.92-7.82 (m, 2H), 7.72 (d, J = 8.1 Hz, 1H), 7.68-7.65 (m, 1H), |
| | 7.65-7.62 (m, 1H), 7.50-7.44 (m, 2H), 7.42-7.19 (m, 9H), 7.15 (d, |
| | J = 2.1 Hz, 1H), 6.85-6.76 (m, 1H), 5.09 (s, 2H), 4.27-4.12 (m, 4H), |
| | 3.21-3.10 (m, 1H), 3.08-2.91 (m, 1H). |
| | LC-MS (ESI, m/z): [M + H]⁺ 533 |
| 3- 23- 24 | 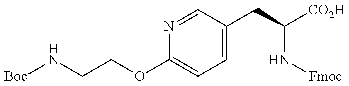 |
| | (S)-2-((((9H-fluoren-9-yl)methoxy)carbonyl)amino)-3-(6-(2-((tert- |
| | butoxycarbonyl)amino)ethoxy)pyridin-3-yl)propanoic acid |
| | methyl (2R)-2-({[(9H-fluoren-9-yl)methoxy]carbonyl}amino)-3- |
| | idoopropanoate (CAS: 156017-42-4) |
| | tert-butyl (2-((5-bromopyridin-2-yl)oxy)ethyl)carbamate (CAS: |
| | 2086299-97-8) |
| | ¹H NMR (400 MHz, DMSO-d₆) δ 12.94 (br, 1H), 8.01 (s, 1H), |
| | 7.94-7.82 (m, 2H), 7.74-7.48 (m, 4H), 7.46-7.37 (m, 2H), |
| | 7.36-7.25 (m, 2H), 7.01-6.92 (m, 1H), 6.76-6.67 (m, 1H), 4.27- |
| | 4.06 (m, 6H), 3.33-3.22 (m, 2H), 3.08-2.98 (m, 1H), 2.88-2.76 |
| | (m, 1H), 1.37 (s, 9H). LC-MS (ESI, m/z): [M + H]⁺ 548. |
| 3- 23- 25 | 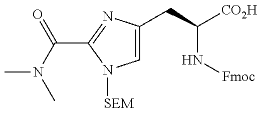 |
| | (S)-2-((((9H-fluoren-9-yl)methoxy)carbonyl)amino)-3-(2- |
| | (dimethylcarbamoyl)-1-((2-(trimethylsilyl)ethoxy)methyl)-1H- |
| | imidazol-4-yl)propanoic acid |
| | methyl (2R)-2-({[9H-fluoren-9-yl)methoxy]carbonyl}amino)-3- |
| | iodopropanoate (CAS: 156017-42-4) |
| | 4-iodo-N,N-dimethyl-1-((2-(trimethylsilyl)ethoxy)methyl)-1H- |
| | imidazole-2-carboxamide |
| | ¹H NMR (500 MHz, CDCl₃) δ 7.77 (d, J = 7.6 Hz, 2H), |
| | 7.66-7.54 (m, 2H), 7.45-7.35 (m, 2H), 7.25-7.28 (m, 2H), 6.96 (s, |
| | 1H), 6.15 (d, J = 5.65 Hz, 1H), 5.56-5.42 (m, 2H), 4.58-4.44 (m, |
| | 1H), 4.44-4.36 (m, 2H), 4.26-4.18 (m, 1H), 3.59-3.46 (m, 2H), |
| | 3.30-3.19 (m, 1H), 3.19-2.96 (m, 7H), 0.94-0.85 (m, 2H), |
| | −0.01-−0.03 (m, 9H). LC-MS (ESI, m/z): [M + H]⁺ 579. |
| 3- 23- 26 | 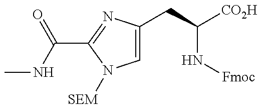 |
| | (S)-2-((((9H-fluoren-9-yl)methoxy)carbonyl)amino)-3-(2- |
| | (methylcarbamoyl)-1-((2-(trimethylsilyl)ethoxy)methyl)-1H- |
| | imidazol-4-yl)propanoic acid |
| | methyl (2R)-2-({[(9H-fluoren-9-yl)methoxy]carbonyl}amino)-3- |
| | iodopropanoate (CAS: 156017-42-4) |
| | 4-iodo-N-methyl-1-((2-(trimethylsilyl)ethoxy)methyl)-1H- |
| | imidazole-2-carboxamide |
| | ¹H NMR (500 MHz, CDCl₃) δ 8.46-8.27 (br, 1H), 7.82 (d, J = 7.5 |
| | Hz, 2H), 7.72-7.58 (m, 2H), 7.50-7.41 (m, 2H), 7.41-7.32 (m, |
| | 2H), 6.92 (s, 1H), 6.04-5.93 (m, 1H), 5.83-5.69 (m, 1H), 5.51 (d, |
| | J = 6.71 Hz, 1H), 4.69-4.56 (m, 2H), 4.52-4.37 (m, 1H), |
| | 4.33-4.22 (m, 1H), 3.66-3.56 (m, 2H), 3.37-3.22 (m, 2H), |
| | 2.92-2.78 (m, 3H), 1.00-0.84 (m, 2H), |
| | 0.03-−0.04 (m, 9H). LC-MS (ESI, m/z): [M + H]⁺ 565. |
| 3- 23- 27 | 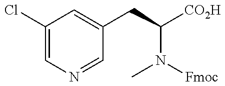 |
| | (S)-2-((((9H-fluoren-9-yl)methoxy)carbonyl)(methyl)amino)-3-(5- |
| | chloropyridin-3-yl)propanoic acid |
| | Reference example-3-1-01 |
| | 3-bromo-5-chloropyridine (CAS: 73583-39-8) |
| | ¹H NMR (500 MHz, DMSO-d₆) δ 13.03 (br, 1H), 8.52-8.22 |
| | (m, 2H), 7.93-7.67 (m, 3H), 7.62-7.37 (m, 4H), 7.35-7.19 (m, |
| | 2H), 4.97-4.69 (m, 1H), 4.44-4.06 (m, 3H), 3.30-2.81 (m, |
| | 2H), 2.77-2.63 (m, 3H). LC-MS (ESI, m/z): [M + H]⁺ 437. |
| 3- 23- 28 | 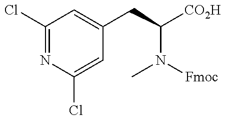 |
| | (2S)-3-(2,6-dichloropyridin-4-yl)-2-({[(9H-fluoren-9- |
| | yl)methoxy]carbonyl}(methyl)amino)propanoic acid |
| | Reference example-3-1-01 |
| | 2,6-dichloro-4-iodopyridine (CAS: 98027-84-0) |
| | ¹H NMR (500 MHz, DMSO-d₆) δ 13.12 (br, 1H), 7.94-7.83 (m, |
| | 2H), 7.64-7.24 (m, 8H), 5.05-4.71 (m, 1H), 4.46-4.12 (m, |
| | 3H), 3.31-2.78 (m, 2H), 2.75-2.60 (m, 3H). |
| | LC-MS (ESI, m/z): [M + H]⁺ 417. |
| 3- 23- 29 | 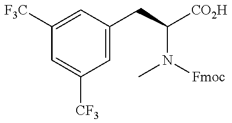 |
| | (2S)-3-[3,5-bis(trifluoromethyl)pehnyl]-2-({[(9H-fluoren-9- |
| | yl)methoxy]carbonyl}(methyl)amino)propanoic acid |
| | Reference example-3-1-01 |
| | 1-bromo-3,5-bis(trifluoromethyl)benzene (CAS: 328-70-1) |
| | ¹H NMR (500 MHz, DMSO-d₆) δ 13.07 (br, 1H), 8.02-7.91 (m, |
| | 2H), 7.91-7.81 (m, 3H), 7.61-7.34 (m, 4H), 7.32-7.16 (m, 2H), |
| | 5.11-4.92 (m, 1H), 4.33-4.17 (m, 1H), 4.16-4.08 (m, 2H), |
| | 3.48-3.39 (m, 1H), 3.32-3.07 (m, 1H), 2.72-2.65 (m, 3H). |
| | LC-MS (ESI, m/z): [M + H]⁺ 538. |
| 3- 23- 30 | 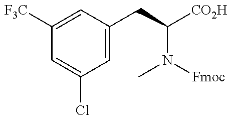 |
| | (2S)-3-[3-chloro-5-(trifluoromethyl)phenyl]-2-({[9H-fluoren-9- |
| | yl)methoxy]carbonyl}(methyl)amino)proapnoic acid |
| | Reference example-3-1-01 |
| | 1-bromo-3-chloro-5-(trifluoromethyl)benzene (CAS: 928783-85-1) |
| | ¹H NMR (500 MHz, DMSO-d₆) δ 13.02 (br, 1H), 7.91-7.83 (m, |
| | 2H), 7.69 (br, 1H), 7.67-7.35 (m, 6H), 7.33-7.21 (m, 2H), 5.02-4.81 |
| | (m, 1H), 4.36-4.21 (m, 1H), 4.19-4.06 (m, 2H), 3.37-2.93 (m, |
| | 2H), 2.74-2.65 (m, 3H),. LC-MS (ESI, m/z): [M + H]⁺ 504. |

### Example-3-24

The following compounds can be synthesized as outlined for the preparation of Example-3-4-07 or Example-3-16-01 or the methods described in Organic Letters (2019), 21(9), 3178. employing appropriate starting materials in the table.

| | Structure and IUPAC name |
|---|---|
| | Starting material-A |
| | Starting material-B |
| Example | NMR and MS |
| 3-24-01 | 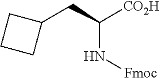 |
| | (2S)-3-cyclobuty1-2-({[(9H-fluoren-9- |
| | yl)methoxy] carbonyl}amino)propanoic acid |
| | 9-chloro-1-[(2S)-1-[(3,4-dichlorophenyl)methyl]pyrrolidine-2-carbonyl]-7- |
| | phenyl-1H,4H,5H-benzo[d]1-oxa-3,7-diaza-2-nickelacyclononan-4-one |
| | (CAS: 2089661-84-5) |
| | (bromomethyl)cyclobutane (CAS: 17247-58-4) |
| | ¹H NMR (400 MHZ, DMSO-d₆) δ 12.54 (br, 1H), 7.90 (dd, J = 9.5 Hz, 2H), |
| | 7.76-7.55 (m, 3H), 7.46-7.28 (m, 4H), 4.34-4.17 (m, 3H), 3.88-3.76 (m, |
| | 1H), 2.42-2.28 (m, 1H), 2.06-1.88 (m, 2H), 1.88-1.48 (m, 2H). LC-MS |
| | (ESI, m/z): [M - H]⁻364. |
| 3-24-02 | 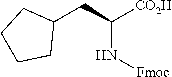 |
| | (2S)-3-cyclopenty1-2-({[(9H-fluoren-9- |
| | yl)methoxy]carbonyl}amino)propanoic acid |
| | 9-chloro-1-[(2S)-1-[(3,4-dichlorophenyl)methyl]pyrrolidine-2-carbonyl]-7- |
| | phenyl-1H,4H,5H-benzo[d]1-oxa-3,7-diaza-2-nickelacyclononan-4-one |
| | (CAS: 2089661-84-5) |
| | (bromomethyl)cyclopentane (CAS: 3814-30-0) |
| | ¹H NMR (500 MHZ, DMSO-d₆) δ 12.52 (br, 1H), 7.90 (d, J = 7.5 Hz, 2H), |
| | 7.75-7.61 (m, 3H), 7.45-7.27 (m, 4H), 4.35-4.17 (m, 3H), 3.97-3.84 (m, |
| | 1H), 1.91-1.78 (m, 1H), 1.78-1.32 (m, 8H), 1.17-0.95 (m, 2H). LC-MS |
| | (ESI, m/z): [M + H]⁺ 380. |
| 3-24-03 | 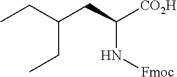 |
| | (2S)-4-ethyl-2-({[(9H-fluoren-9-yl)methoxy]carbonyl}amino)hexanoic |
| | acid |
| | 9-chloro-1-[(2S)-1-[(3,4-dichlorophenyl)methyl]pyrrolidine-2-carbonyl]-7- |
| | phenyl-1H,4H,5H-benzo[d]1-oxa-3,7-diaza-2-nickelacyclononan-4-one |
| | (CAS: 2089661-84-5) |
| | 3-(bromomethyl)pentane (CAS: 3814-34-4) |
| | ¹H NMR (500 MHZ, DMSO-d₆) δ 12.56 (br, 1H), 7.90 (d, J = 7.5 Hz, 2H), |
| | 7.74-7.58 (m, 3H), 7.48-7.12 (m, 4H), 4.36-4.18 (m, 3H), 4.06-3.90 (m, |
| | 1H), 1.63-1.48 (m, 2H), 1.40-1.12 (m, 5H), 0.89-0.72 (m, 6H). LC-MS |
| | (ESI, m/z): [M + H]⁺ 382. |
| 3-24-04 | 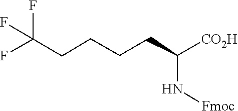 |
| | (2S)-2-({[(9H-fluoren-9-yl)methoxy]carbonyl}amino)-7,7,7- |
| | trifluoroheptanoic acid |
| | 9-chloro-1-[(2S)-1-[(3,4-dichlorophenyl)methyl]pyrrolidine-2-carbonyl]-7- |
| | phenyl-1H,4H,5H-benzo[d]1-oxa-3,7-diaza-2-nickelacyclononan-4-one |
| | (CAS: 2089661-84-5) |
| | 5-bromo-1,1,1-trifluoropentane (CAS: 54932-74-0) |
| | ¹H NMR (500 MHz, DMSO-d₆) δ 12.56 (br, 1H), 7.90 (d, J =7.5 Hz, 2 H), |
| | 7.77-7.67 (m, 2H), 7.63 (d, J =8.0 Hz, 1 H), 7.42 (d, J =7.5 Hz, 2H), 7.33 |
| | (d, J =8.0 Hz, 2H), 4.35-4.16 (m, 3H), 3.97-3.81 (m, 1H), 2.30-2.14 (m, |
| | 2H), 1.79-1.68 (m, 1H), 1.68-1.56 (m, 1H), 1.56-1.30 (m, 4H). LC-MS |
| | (ESI, m/z): [M + H]⁺ 422. |
| 3-24-05 | 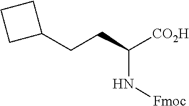 |
| | (2S)-4-cyclobuty1-2-({[(9H-fluoren-9- |
| | yl)methoxy] carbonyl}amino)butanoic acid |
| | 9-chloro-1-[(2S)-1-[(3,4-dichlorophenyl)methyl]pyrrolidine-2-carbonyl]-7- |
| | phenyl-1H,4H,5H-benzo[d]1-oxa-3,7-diaza-2-nickelacyclononan-4-one |
| | (CAS: 2089661-84-5) |
| | (2-bromoethyl)cyclobutane (CAS: 960078-90-4) |
| | ¹H NMR (500 MHZ, DMSO-d₆) δ 12.54 (br, 1H), 7.90 (d, J = 7.5 Hz, 2H), |
| | 7.73 (d, J = 7.0 Hz, 2H), 7.63 (d, J =8.0 Hz, 1H), 7.45-7.39 (m, 2H), 7.36 |
| | -7.30 (m, 2H), 4.31-4.17 (m, 3H), 3.91-3.85 (m, 1H), 2.25-2.16 (m, 1H), |
| | 2.04-1.95 (m, 2H), 1.95-1.66 (m, 2H), 1.66-1.35 (m, 6H). LC-MS (ESI, |
| | m/z): [M + H]⁺ 380. |
| 3-24-06 | 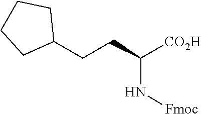 |
| | (2S)-4-cyclopenty1-2-({[(9H-fluoren-9- |
| | yl)methoxy] carbony1}amino)butanoic acid |
| | 9-chloro-1-[(2S)-1-[(3,4-dichlorophenyl)methyl]pyrrolidine-2-carbonyl]-7- |
| | phenyl-1H,4H,5H-benzo[d]1-oxa-3,7-diaza-2-nickelacyclononan-4-one |
| | (CAS: 2089661-84-5) |
| | (2-bromoethyl)cyclopentane (CAS: 18928-94-4) |
| | ¹H NMR (500 MHz, DMSO-d₆) δ 12.54 (br, 1H), 7.89 (d, J = 7.5 Hz, 2H), |
| | 7.81-7.52 (m, 3H), 7.52-7.23 (m, 4H), 4.36-4.18 (m, 3H), 3.98-3.80 (m, |
| | 1H), 1.89-1.40 (m, 9H), 1.40-1.20 (m, 2H), 1.20-0.89 (m, 2H). LC-MS |
| | (ESI, m/z): [M + Na]⁺ 416. |
| 3-24-07 | 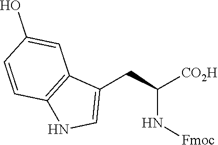 |
| | (2S)-2-({[(9H-fluoren-9-yl)methoxy]carbonyl}amino)-3-(5-hydroxy-1H- |
| | indol-3-yl)propanoic acid |
| | (2R)-3,6-dimethoxy-2-(propan-2-yl)-2,5-dihydropyrazine (CAS: 109838- |
| | 85-9) |
| | tert-butyl 3-(bromomethyl)-5-{[(tert-butoxy)carbonyl]oxy}-1H-indole-1- |
| | carboxylate (CAS: 390815-81-3) |
| | ^{I}H NMR (500 MHz, DMSO-d₆) δ 12.61 (br, 1H), 10.53 (br, 1H), 8.62 (br, |
| | 1H), 7.95-7.81 (m, 2H), 7.72-7.56 (m, 3H), 7.52-7.36 (m, 2H), 7.34 - |
| | 7.24 (m, 2H), 7.17-7.11 (m, 1H), 7.10-7.05 (m, 1H), 6.90-6.79 (m, 1H), |
| | 6.67-6.52 (m, 1H), 4.33-3.97 (m, 4H), 3.14-3.03 (m, 1H), 2.97-2.85 (m, |
| | 1H). LC-MS (ESI, m/z): [M + H]⁺ 443. |
| 3-24-08 | 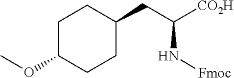 |
| | (2S)-2-({[(9H-fluoren-9-y1)methoxy]carbonyl}amino)-3-[(1r,4r)-4- |
| | methoxy cyclohexy1]propanoic acid |
| | (2R)-3,6-dimethoxy-2-(propan-2-y1)-2,5-dihydropyrazine (CAS: 109838- |
| | 85-9) 5 |
| | (1r,4r)-1-(bromomethy1)-4-methoxycyclohexane (CAS: 2196175-77-4) |
| | ¹H NMR (300 MHZ, DMSO-d₆) δ 12.57 (br, 1H), 7.92 (d, J = 7.4 Hz, 2H), |
| | 7.76-771 (m, 2H), 7.65 (d, J = 8.3 Hz, 1H), 7.47-7.40 (m, 2H), 7.39-7.30 |
| | (m, 2H), 4.37-4.21 (m, 3H), 4.05-3.94 (m, 1H), 3.23 (s, 3H), 3.10-2.99 |
| | (m, 1H), 2.05-1.93 (m, 2H), 1.80-1.45 (m, 4H), 1.40-1.24 (m, 1H), 1.15 |
| | -0.81 (m, 4H). LC-MS (ESI, m/z): [M + Na]⁺ 446. |
| 3-24-09 | 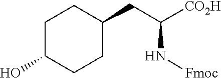 |
| | (2S)-2-({[(9H-fluoren-9-y1)methoxy]carbonyl}amino)-3-[(1r,4r)-4- |
| | hydroxy cyclohexyl]propanoic acid |
| | (2R)-3,6-dimethoxy-2-(propan-2-y1)-2,5-dihydropyrazine (CAS: 109838- |
| | 85-9) |
| | tert-butyldimethyl {[(1r,4r)-4-(bromomethyl)cyclohexyl]oxy}silane (CAS: |
| | 1356347-22-2) |
| | ¹H NMR (300 MHZ, Methanol-d₄) δ 7.80 (d, J = 7.35 Hz, 2H), 7.72-7.65 |
| | (m, 2H), 7.64-7.57 (m, 1H), 7.45-7.25 (m, 4H), 4.48-4.31 (m, 2H), 4.27 |
| | -4.17 (m, 2H), 3.54-3.42 (m, 1H), 1.98-1.73 (m, 4H), 1.69-1.58 (m, 2H), |
| | 1.54-1.14 (m, 4H), 1.14-0.88 (m, 2H). LC-MS (ESI, m/z): [M + Na]⁺ 432. |
| 3-24-10 | 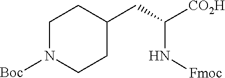 |
| | (2R)-3-{1-[(tert-butoxy)carbonyl]piperidin-4-y1}-2-({[(9H-fluoren-9- |
| | y1)methoxy] carbonyl}amino)propanoic acid |
| | 9-chloro-1-[(2R)-1-[(3,4-dichlorophenyl)methyl]pyrrolidine-2-carbonyl]-7- |
| | phenyl-1H,4H,5H-benzo[d]1-oxa-3,7-diaza-2-nickelacyclononan-4-one |
| | (CAS: 2408623-29-8) |
| | tert-butyl 4-(bromomethyl)piperidine-1-carboxylate (CAS: 158407-04-6) |
| | ¹H NMR (300 MHZ, DMSO-d₆) δ 12.57 (br, 1H), 7.89 (d, J = 7.5 Hz, 2H), |
| | 7.78-7.69 (m, 2H), 7.67-7.57 (m, 1H), 7.47-7.37 (m, 2H), 7.37-7.26 (m, |
| | 2H), 4.40-4.18 (m, 3H), 4.07-3.84 (m, 3H), 2.78-2.53 (m, 2H), 1.70 - |
| | 1.44 (m, 5H), 1.38 (s, 9H), 1.13-0.80 (m, 2H). LC-MS (ESI, m/z): |
| | [M - Boc + H]⁺ 395. |

### Example-3-25

The following compounds can be synthesized as outlined for the preparation of Reference example-3-6-01, Example-3-1-01, Example-3-4-01, or Example-3-4-02 employing appropriate starting materials in the table.

| | Structure and IUPAC name |
|---|---|
| | Starting material-A |
| Example | NMR and MS |
| 3-25-01 | 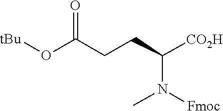 |
| | (2S)-5-(tert-butoxy)-2-({[(9H-fluoren-9- |
| | yl)methoxy]carbonyl}(methyl)amino)-5-oxopentanoic acid |
| | (2S)-2-{[(benzyloxy)carbonyl]amino}-5-(tert-butoxy)-5-oxopentanoic acid |
| | (CAS: 3886-08-6) |
| | ¹H NMR (300 MHZ, DMSO-d₆) δ 12.84 (br, 1H), 7.93-7.84 (m, 2H), 7.69 |
| | -7.59 (m, 2H), 7.46-7.27 (m, 4H), 4.54-4.20 (m, 4H), 2.72 (s, 3H), 2.18 - |
| | 1.97 (m, 3H), 1.93-1.74 (m, 1H), 1.38 (s, 9H). LC-MS (ESI, m/z): [M + H]⁺ |
| | 440. |
| 3-25-02 | 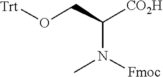 |
| | (2S)-2-({[(9H-fluoren-9-yl)methoxy]carbonyl}(methyl)amino)-3- |
| | (triphenylmethoxy)propanoic acid |
| | (2S)-2-{[(benzyloxy)carbonyl]amino}-3- |
| | [(triphenylmethyl)sulfany1]propanoic acid (CAS: 1330286-53-7) |
| | ¹H NMR (400 MHZ, DMSO-d₆) δ 13.06 (br, 1H), 7.98-7.84 (m, 2H), 7.73 |
| | -7.59 (m, 2H), 7.49-7.19 (m, 19H), 4.91-4.77 (m, 1H), 4.51-4.09 (m, |
| | 3H), 3.52-3.20 (m, 2H), 2.89-2.67 (m, 3H). LC-MS (ESI, m/z): [M + Na]⁺ |
| | 606. |
| 3-25-03 | 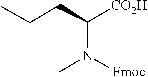 |
| | (2S)-2-({[(9H-fluoren-9-yl)methoxy]carbonyl}(methyl)amino)pentanoic acid |
| | (2S)-2-({[(9H-fluoren-9-y1)methoxy]carbonyl}amino)pentanoic acid (CAS: |
| | 135112-28-6) |
| | ¹H NMR (500 MHz, DMSO-d₆) δ 12.78 (br, 1H), 7.93-7.87 (m, 2H), 7.69 |
| | -7.58 (m, 2H), 7.47-7.27 (m, 4H), 4.59-4.20 (m, 4H), 2.75-2.61 (m, 3H), |
| | 1.81-1.49 (m, 2H), 1.31-0.95 (m, 2H), 0.93-0.74 (m, 3H). LC-MS (ESI, |
| | m/z): [M + H]⁺ 354. |

### Example-3-26-01: (2S)-3-{3-[(3-{[(tert-butoxy)carbonyl]amino}propyl)carbamoyl]phenyl}-2-({[(9H-fluoren-9-yl)methoxy]carbonyl}(methyl)amino)propanoic acid

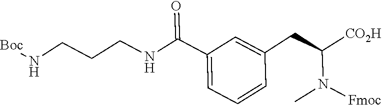

To a solution of (S)-2-((((9H-fluoren-9-yl)methoxy)carbonyl)(methyl)amino)-3-(3-(tert-butoxycarbonyl)phenyl)propanoic acid (CAS: 210282-33-0, 50 g, 100 mmol) in DMF (399 mL) at 0° C. were added DIPEA (22.6 mL, 130 mmol) and allyl bromide (10.4 mL, 120 mmol). The mixture was stirred at rt for 16 h. The reaction mixture at 0° C. was quenched with H₂O. The aqueous phase was extracted with hexane/ethyl acetate (1/1). The combined organic layer was washed with H₂O and brine, dried over Na₂SO₄ and filtered, and concentrated. To a solution of the resulting residue in DCM (330 mL) was added TFA (160 mL). The mixture was stirred at rt for 2 h, and then concentrated. The resulting residue was suspended in toluene, and then concentrated (repeated three times). To a solution of the resulting residue, tert-butyl (3-aminopropyl)carbamate (CAS: 75178-96-0, 26.1 g, 150 mmol), and DIPEA (21.0 mL, 120 mmol) in DMF (500 mL) was added HATU (45.6 g, 120 mmol). The mixture was stirred at rt for 6 h. To the mixture was added tert-butyl (3-aminopropyl)carbamate (7.0 g, 40 mmol), DIPEA (7.0 mL, 40 mmol) and HATU (15.3 g, 40 mmol). The mixture was stirred at rt for 14 h, and then concentrated. The resulting residue was dissolved in ethyl acetate, and then washed successively with H₂O and brine, dried over Na₂SO₄, filtered, and then concentrated. The resulting residue was suspended in DCM, and then filtered. The filtrate was concentrated. To a solution of the resulting residue (64.2 g, 100 mmol, theoretical) in DCM (46 mL) at 0° C. was added phenylsilane (24.7 mL, 200 mmol), followed by Pd(PPh₃)₄ (5.8 g, 5 mmol). The mixture was stirred at rt for 18 h, and then concentrated. The resulting residue was purified by silica gel flash column chromatography (gradient, CH₂Cl₂/MeOH=100/0 to 90/10, and then CH₂Cl₂/EtOAc=50/50). Appropriate fractions were collected and concentrated. The residual sample was dissolved in CH₃CN, and then concentrated. The sample was dried under the high vac. to afford the title compound (35.2 g, 58.5 mmol). ¹H NMR (500 MHz, DMSO-d₆) δ 13.05 (br, 1H), 8.41-8.35 (m, 1H), 7.87 (d, J=5.5 Hz, 2H), 7.76 (s, 1H), 7.70-7.62 (m, 1H), 7.62-7.14 (m, 8H), 6.86-6.77 (m, 1H), 4.84-4.77 (m, 1H), 4.30-4.07 (m, 3H), 3.50-3.02 (m, 4H), 3.02-2.88 (m, 2H), 2.83-2.61 (m, 3H), 1.61-1.52 (m, 2H), 1.37 (s, 9H). LC-MS (ESI, m/z): [M+H]⁺ 602.

### Example-3-26-02: (2S)-3-{3-[(2-{[(tert-butoxy)carbonyl]amino}ethyl)carbamoyl]phenyl}-2-({[(9H-fluoren-9-yl)methoxy]carbonyl}(methyl)amino)propanoic acid

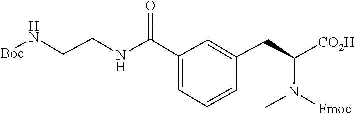

The title compound can be synthesized as outlined for the preparation of Example-3-23-01 employing appropriate reagent tert-butyl (3-aminoethyl)carbamate (CAS: 75178-96-0). ¹H NMR (500 MHz, DMSO-d₆) δ 13.06 (br, 1H), 8.46-8.36 (m, 1H), 7.87 (d, J=7.5 Hz, 2H), 7.76 (s, 1H), 7.71-7.05 (m, 9H), 6.86-6.77 (m, 1H), 4.84-4.77 (m, 1H), 4.28-4.07 (m, 3H), 3.45-3.15 (m, 4H), 3.15-3.03 (m, 2H), 2.81-2.62 (m, 3H), 1.39-1.33 (s, 9H). LC-MS (ESI, m/z): [M+H]⁺ 588.

### Example-3-27-01: (2S,3S)-3-(tert-butoxy)-2-({[(9H-fluoren-9-yl)methoxy]carbonyl}amino)butanoic acid

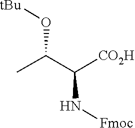

The title compound can be synthesized as outlined for the preparation methods described in Nature chemistry (2010), 2(4), 280. employing appropriate starting material (2R,3R)-2-amino-3-hydroxybutanoic acid (CAS: 24830-94-2). ¹H NMR (500 MHz, DMSO-d₆) δ 12.70 (br, 1H), 7.90 (d, J=7.5 Hz, 2H), 7.78-7.74 (m, 2H), 7.57-7.49 (m, 1H), 7.44-7.40 (m, 2H), 7.35-7.30 (m, 2H), 4.31-4.20 (m, 3H), 4.14-4.06 (m, 1H), 4.03-3.96 (m, 1H), 1.12 (s, 9H), 1.07 (d, J=6.3 Hz, 3H). LC-MS (ESI, m/z): [M+H]⁺ 398.

### Example-3-28

The following compounds can be synthesized as outlined for the preparation of Reference example-3-1-04, Example-3-2-06, Example-3-3-05, or Example-3-7-04 employing appropriate starting materials in the table.

| | Structure and IUPAC name |
|---|---|
| | Starting material-A |
| | Starting material-B |
| Example | NMR and MS |
| 3-28-01 | 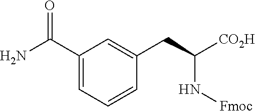 |
| | (2S)-3-(3-carbamoylpheny1)-2-({[(9H-fluoren-9- |
| | yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | 3-bromobenzamide (CAS: 698-67-9) |
| | ¹H NMR (400 MHZ, DMSO-d₆) δ 12.78 (br, 1H), 8.11-7.59 (m, 8H), 7.57 |
| | -7.06 (m, 7H), 4.42-3.99 (m, 4H), 3.24-3.08 (m, 1H), 3.03-2.81 (m, 1H). |
| | LC-MS (ESI, m/z): [M + H]⁺ 431. |
| 3-28-02 | 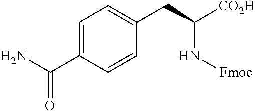 |
| | (2S)-3-(4-carbamoylpheny1)-2-({[(9H-fluoren-9- |
| | yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | 4-bromobenzamide (CAS: 698-67-9) |
| | ¹H NMR (400 MHZ, DMSO-d₆) δ 12.79 (br, 1H), 8.03-7.73 (m, 6H), 7.72 |
| | -7.22 (m, 9H), 4.34-4.10 (m, 4H), 3.24-3.06 (m, 1H), 3.02-2.76 (m, 1H). |
| | LC-MS (ESI, m/z): [M + H]⁺ 431. |
| 3-28-03 | 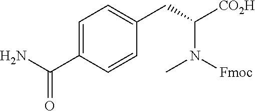 |
| | (2R)-3-(4-carbamoylpheny])-2-({[(9H-fluoren-9- |
| | yl)methoxy]carbonyl}(methyl)amino)propanoic acid |
| | Reference example-3-1-02 |
| | 4-bromobenzamide (CAS: 698-67-9) |
| | ¹H NMR (300 MHZ, DMSO-d₆) δ 12.92 (br, 1H), 8.06-6.99 (m, 14H), 4.93 |
| | -4.61 (m, 1H), 4.40-4.11 (m, 3H), 3.30-2.80 (m, 2H), 2.78-2.59 (m, 3H). |
| | LC-MS (ESI, m/z): [M + H]⁺ 445. |
| 3-28-04 | 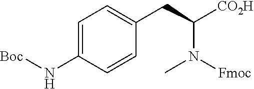 |
| | (2S)-3-(4-{[(tert-butoxy)carbonyl]amino}phenyl)-2-({[(9H-fluoren-9- |
| | yl)methoxy] carbonyl}(methyl)amino)propanoic acid |
| | Reference example-3-1-01 |
| | tert-butyl N-(4-bromophenyl)carbamate (CAS: 131818-17-2) |
| | ¹H NMR (300 MHZ, DMSO-d₆) δ 12.84 (br, 1H), 9.24 (s, 1H), 7.88 (d, J = |
| | 7.4 Hz, 2H), 7.72-7.24 (m, 8H), 7.14-6.90 (m, 2H), 4.78-4.61 (m, 1H), |
| | 4.35-4.11 (m, 3H), 3.24-2.61 (m, 5H), 1.64-1.18 (m, 9H). LC-MS (ESI, |
| | m/z): [M - H]⁻ 515. |
| 3-28-05 | 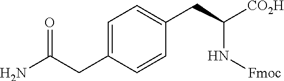 |
| | (S)-2-((((9H-fluoren-9-yl)methoxy)carbonyl)amino)-3-(4-(2-amino-2- |
| | oxoethyl)phenyl )propanoic acid |
| | methyl (2R)-2-({[(9H-fluoren-9-y1)methoxy]carbonyl}amino)-3- |
| | iodopropanoate (CAS: 156017-42-4) |
| | 2-(4-bromophenyl)acetamide (CAS: 74860-13-2) |
| | ¹H NMR (500 MHz, DMSO-d₆) δ 7.92-7.85 (m, 2H), 7.69-7.62 (m, 2H), |
| | 7.48-7.25 (m, 6H), 7.18-7.10 (m, 4H), 6.85 (br, 1H), 4.27-4.13 (m, 3H), |
| | 4.02 (br, 1H), 3.32-3.30 (m, 2H), 3.09-2.97 (m, 1H), 2.93-2.79 (m, 1H). |
| | LC-MS (ESI, m/z): [M + H]⁺ 445. |
| 3-28-06 | 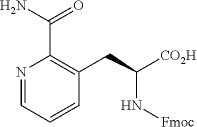 |
| | (S)-2-((((9H-fluoren-9-yl)methoxy)carbonyl)amino)-3-(2- |
| | carbamoylpyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-Bromo-2-pyridinecarboxamide (CAS: 36057-54-2) |
| | ¹H NMR (500 MHz, DMSO-d₆) δ 12.71 (br, 1H), 8.50-8.47 (m, 1H), 8.13 |
| | (br, 1H), 7.93-7.84 (m, 3H), 7.82-7.76 (m, 1H), 7.68 (br, 1H), 7.62-7.38 |
| | (m, 5H), 7.34-7.23 (m, 2H), 4.40-4.31 (m, 1H), 4.20-4.05 (m, 3H), 3.66 |
| | -3.51 (m, 1H), 3.15-3.02 (m, 1H). LC-MS (ESI, m/z): [M + H]⁺ 432. |
| 3-28-07 | 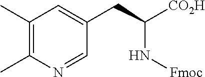 |
| | (S)-2-((((9H-fluoren-9-yl)methoxy)carbonyl)amino)-3-(5,6- |
| | dimethylpyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromo-2,3-dimethylpyridine (CAS: 27063-90-7) |
| | ¹H NMR (300 MHz, DMSO-d₆) δ 13.00 (br, 1H), 8.58-8.50 (m, 1H), 8.31 |
| | -8.20 (m, 1H), 7.96-7.80 (m, 3H), 7.70-7.56 (m, 2H), 7.47-7.37 (m, 2H), |
| | 7.37-7.26 (m, 2H), 4.38-4.09 (m, 4H), 3.31-3.21 (m, 1H), 3.12-2.88 (m, |
| | 1H), 2.67-2.57 (m, 3H), 2.40-2.23 (m, 3H). LC-MS (ESI, m/z): [M + H]⁺ |
| | 417. |
| 3-28-08 | 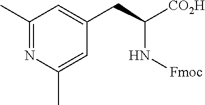 |
| | (S)-2-((((9H-fluoren-9-yl)methoxy)carbonyl)amino)-3-(2,6- |
| | dimethylpyridin-4-yl)propanoic acid |
| | methyl (2R)-2-({[(9H-fluoren-9-yl)methoxy]carbonyl}amino)-3- |
| | iodopropanoate (CAS: 156017-42-4) |
| | 4-bromo-2,6-dimethylpyridine (CAS: 5093-70-9) |
| | ¹H NMR (400 MHZ, DMSO-d₆) δ 13.43-12.70 (m, 1H), 7.89 (d, J = 7.5 Hz, |
| | 2H), 7.83 (d, J = 8.5 Hz, 1H), 7.70-7.58 (m, 2H), 7.45-7.25 (m, 6H), 4.36 |
| | -4.14 (m, 4H), 3.24-3.13 (m, 1H), 3.05-2.89 (m, 1H), 2.54-2.53 (m, 6H). |
| | LC-MS (ESI, m/z): [M + H]⁺ 417. |
| 3-28-09 | 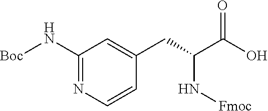 |
| | (R)-2-((((9H-fluoren-9-yl)methoxy )carbonyl)amino)-3-(2-((tert- |
| | butoxy carbonyl)amino)pyridin-4-yl)propanoic acid |
| | methyl (2S)-2-({[(9H-fluoren-9-y1)methoxy]carbonyl}amino)-3- |
| | iodopropanoate (CAS: 527696-77-1) |
| | 4-bromopyridin-2-amine (CAS: 84249-14-9) |
| | ¹H NMR (300 MHz, DMSO-d₆) δ 12.86 (br, 1H), 9.68 (s, 1H), 8.23-8.01 |
| | (m, 1H), 7.88 (d, J = 7.5 Hz, 2H), 7.81-7.73 (m, 2H), 7.68-7.58 (m, 2H), |
| | 7.50-7.37 (m, 2H), 7.34-7.25 (m, 2H), 6.94 (d, J =5.2 Hz, 1H), 4.32-4.08 |
| | (m, 4H), 3.17-3.01 (m, 1H), 2.97-2.79 (m, 1H), 1.55-1.28 (m, 9H). LC- |
| | MS (ESI, m/z): [M + H]⁺ 504. |
| 3-28-10 | 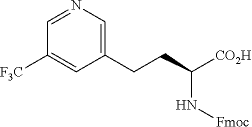 |
| | (S)-2-((((9H-fluoren-9-yl)methoxy)carbonyl)amino)-4-(5- |
| | (trifluoromethyl)pyridin-3-yl)butanoic acid |
| | Reference example-3-1-03 |
| | 3-bromo-5-(trifluoromethyl)pyridine (CAS: 436799-33-6) |
| | ¹H NMR (500 MHZ, DMSO-d₆) δ 8.79-8.73 (m, 1H), 8.72-8.67 (m, 1H), |
| | 7.97 (br, 1H), 7.91-7.86 (m, 2H), 7.72-7.66 (m, 2H), 7.43-7.36 (m, 2H), |
| | 7.34-7.27 (m, 2H), 6.82 (br, 1H), 4.36-4.27 (m, 1H), 4.26-4.18 (m, 2H), |
| | 3.77-3.66 (m, 1H), 2.73-2.62 (m, 2H), 2.06-1.95 (m, 1H), 1.93-1.82 (m, |
| | 1H). LC-MS (ESI, m/z): [M + H]⁺ 471. |
| 3-28-11 | 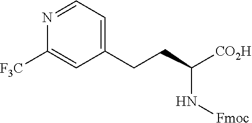 |
| | (S)-2-((((9H-fluoren-9-yl)methoxy )carbonyl)amino)-4-(2- |
| | (trifluoromethyl)pyridin-4-yl)butanoic acid |
| | Reference example-3-1-03 |
| | 4-bromo-2-(trifluoromethyl)pyridine (CAS: 887583-90-6) |
| | ¹H NMR (500 MHZ, DMSO-d₆) δ 12.70 (br, 1H), 8.66 (d, J = 4.9 Hz, 1H), |
| | 7.94-7.87 (m, 2H), 7.81-7.71 (m, 4H), 7.60-7.52 (m, 1H), 7.44-7.39 (m, |
| | 2H), 7.37-7.31 (m, 2H), 4.37-4.30 (m, 2H), 4.28-4.22 (m, 1H), 3.96 - |
| | 3.87 (m, 1H), 2.84-2.72 (m, 2H), 2.13-2.02 (m, 1H), 2.00-1.86 (m, 1H). |
| | LC-MS (ESI, m/z): [M + H]⁺ 471. |
| 3-28-12 | 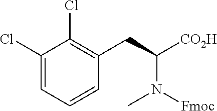 |
| | (S)-2-((((9H-fluoren-9-yl)methoxy )carbonyl)(methyl)amino)-3-(2,3- |
| | dichlorophenyl)propanoic acid |
| | Reference example-3-1-01 |
| | 1-bromo-2,3-dichlorobenzene (CAS: 56961-77-4) |
| | ¹H NMR (300 MHZ, DMSO-d₆) δ 13.07 (br, 1H), 7.93-7.81 (m, 2H), 7.60 |
| | -7.35 (m, 5H), 7.35-7.02 (m, 4H), 4.93-4.75 (m, 1H), 4.29-4.13 (m, 2H), |
| | 3.59-2.98 (m, 3H), 2.72 (s, 3H). LC-MS (ESI, m/z): [M + H]⁺ 470. |
| 3-28-13 | 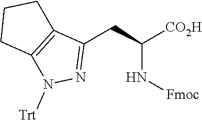 |
| | (S)-2-((((9H-fluoren-9-yl)methoxy)carbonyl)amino)-3-(1-trity1-1,4,5,6- |
| | tetrahydrocyclopenta[ c]pyrazol-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-iodo-1-trityl-1,4,5,6-tetrahydrocyclopenta[c]pyrazole |
| | ¹H NMR (300 MHz, DMSO-d₆) δ 12.68 (br, 1H), 7.90 (d, J = 7.7 Hz, 2H), |
| | 7.72-7.62 (m, 2H), 7.47-7.37 (m, 3H), 7.35-7.24 (m, 11H), 7.12-6.99 |
| | (m, 6H), 4.37-4.12 (m, 4H), 2.98-2.81 (m, 2H), 2.47-2.37 (m, 2H), 2.17 |
| | -2.05 (m, 2H), 1.59-1.41 (m, 2H). LC-MS (ESI, m/z): [M + H]⁺ 660. |
| 3-28-14 | 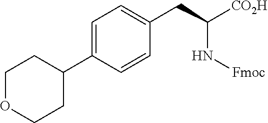 |
| | (S)-2-((((9H-fluoren-9-yl)methoxy)carbonyl)amino)-3-(4-(tetrahydro-2H- |
| | pyran-4-yl)phenyl)propanoic acid |
| | methyl (2R)-2-({[(9H-fluoren-9-yl)methoxy]carbonyl}amino)-3- |
| | iodopropanoate (CAS: 156017-42-4) |
| | 4-(4-bromophenyl)tetrahydro-2H-pyran (CAS: 811828-76-9) |
| | ¹H NMR (400 MHZ, DMSO-d₆) δ 12.78 (br, 1H), 7.88 (d, J = 7.6 Hz, 2H), |
| | 7.72 (d, J = 8.4 Hz, 1H), 7.69-7.62 (m, 2H), 7.44-7.37 (m, 2H), 7.34-7.26 |
| | (m, 2H), 7.23-7.16 (m, 2H), 7.16-7.10 (m, 2H), 4.24-4.11 (m, 4H), 3.94 |
| | -3.86 (m, 2H), 3.44-3.35 (m, 2H), 3.12-2.97 (m, 1H), 2.89-2.79 (m, 1H), |
| | 2.72-2.64 (m, 1H), 1.69-1.52 (m, 4H). LC-MS (ESI, m/z): [M + Na]⁺ 494. |
| 3-28-15 | 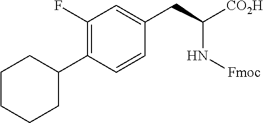 |
| | (2S)-2-({[(9H-fluoren-9-yl)methoxy]carbonyl}amino)-3-{2-fluoro-[1,1'- |
| | bipheny1]-4-yl}propanoic acid |
| | methyl (2R)-2-({[(9H-fluoren-9-yl)methoxy]carbonyl}amino)-3- |
| | iodopropanoate (CAS: 156017-42-4) |
| | 4-bromo-2-fluoro-1,1'-biphenyl (CAS: 41604-19-7) |
| | ¹H NMR (500 MHZ, DMSO-d₆) δ 12.84 (br, 1H), 7.93-7.84 (m, 2H), 7.87 |
| | -7.80 (m, 1H), 7.69-7.61 (m, 2H), 7.57-7.35 (m, 8H), 7.34-7.15 (m, 4H), |
| | 4.30-4.12 (m, 4H), 3.21-3.12 (m, 1H), 2.98-2.88 (m, 1H). LC-MS (ESI, |
| | m/z): [M + H]⁺ 482. |
| 3-28-16 | 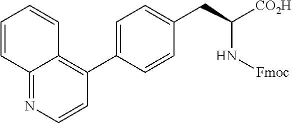 |
| | (S)-2-((((9H-fluoren-9-y1)methoxy)carbonyl)amino)-3-(4-(quinolin-4- |
| | yl)phenyl)propanoic acid |
| | methyl (2R)-2-({[(9H-fluoren-9-yl)methoxy]carbonyl}amino)-3- |
| | iodopropanoate (CAS: 156017-42-4) |
| | 4-(4-bromophenyl)quinoline (CAS: 1622876-34-9) |
| | ¹H NMR (500 MHZ, DMSO-d₆) δ 12.88 (br, 1H), 9.13-9.05 (m, 1H), 8.22 |
| | -8.15 (m, 1H), 7.97-7.84 (m, 5H), 7.71-7.59 (m, 4H), 7.59-7.47 (m, 4H), |
| | 7.44-7.35 (m, 2H), 7.34-7.24 (m, 2H), 4.36-4.13 (m, 4H), 3.33-3.18 (m, |
| | 1H), 3.11-2.97 (m, 1H). LC-MS (ESI, m/z): [M + H]⁺ 515. |
| 3-28-17 | 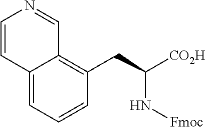 |
| | (S)-2-((((9H-fluoren-9-yl)methoxy)carbonyl)amino)-3-(isoquinolin-8- |
| | yl)propanoic acid |
| | methyl (2R)-2-({[(9H-fluoren-9-y1)methoxy]carbonyl}amino)-3- |
| | iodopropanoate (CAS: 156017-42-4) |
| | 8-bromoisoquinoline (CAS: 63927-22-0) |
| | ¹H NMR (300 MHZ, DMSO-d₆) δ 13.01 (br, 1H), 9.75 (s, 1H), 8.68-8.55 |
| | (m, 1H), 8.20-8.07 (m, 1H), 8.07-7.51 (m, 8H), 7.50-7.20 (m, 4H), 4.51 |
| | -4.32 (m, 1H), 4.25-3.96 (m, 3H), 3.88-3.73 (m, 1H), 3.56-3.37 (m, 1H). |
| | LC-MS (ESI, m/z): [M + H]⁺ 439. |
| 3-28-18 | 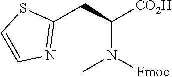 |
| | (S)-2-((((9H-fluoren-9-yl)methoxy)carbonyl)(methyl)amino)-3-(thiazol-2- |
| | yl)propanoic acid |
| | Reference example-3-1-01 |
| | 2-iodothiazole (CAS: 3034-54-6) |
| | ¹H NMR (400 MHZ, CD₃OD) d 7.86-7.76 (m, 2H), 7.71-7.56 (m, 3H), |
| | 7.48-7.27 (m, 5H), 4.99-4.84 (m, 1H), 4.40-4.27 (m, 1H), 4.24-4.14 (m, |
| | 1H), 3.82-3.73 (m, 1H), 3.56-3.36 (m, 2H), 2.95-2.89 (m, 1H). LC-MS |
| | (ESI, m/z): [M + H]⁺ 409. |
| 3-28-19 | 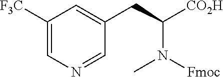 |
| | (S)-2-((((9H-fluoren-9-yl)methoxy)carbonyl)(methyl)amino)-3-(5- |
| | (trifluoromethy1)pyridin-3-yl)propanoic acid |
| | Reference example-3-1-01 |
| | 3-bromo-5-(trifluoromethyl)pyridine (CAS: 436799-33-6) |
| | ¹H NMR (500 MHZ, DMSO-d₆) δ 13.04 (br, 1H), 8.76 (m, 2H), 8.15-7.83 |
| | (m, 3H), 7.62-7.22 (m, 6H), 5.03-4.77 (m, 1H), 4.37-4.06 (m, 3H), 3.38 |
| | -2.95 (m, 2H), 2.77-2.63 (m, 3H). LC-MS (ESI, m/z): [M + H]⁺ 471. |
| 3-28-20 | 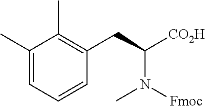 |
| | (2S)-3-(2,3-dimethylpheny1)-2-({[(9H-fluoren-9- |
| | y1)methoxy]carbonyl}(methyl)amino)propanoic acid |
| | Reference example-3-1-01 |
| | 1-iodo-2,3-dimethylbenzene (CAS: 31599-60-7) |
| | ¹H NMR (500 MHZ, DMSO-d₆) δ 12.90 (br, 1H), 7.94-7.81 (m, 2H), 7.59 |
| | -7.36 (m, 4H), 7.35-7.22 (m, 2H), 7.06-6.75 (m, 3H), 4.69-4.59 (m, 1H), |
| | 4.31-4.05 (m, 3H), 3.29-3.14 (m, 1H), 3.08-2.83 (m, 1H), 2.74-2.66 (m, |
| | 3H), 2.24-2.05 (m, 6H). LC-MS (ESI, m/z): [M + H]⁺ 430. |
| 3-28-21 | 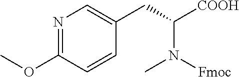 |
| | (2R)-2-({[(9H-fluoren-9-yl)methoxy]carbonyl}(methyl)amino)-3-(6- |
| | methoxypyridin-3-yl)propanoic acid |
| | Reference example-3-1-02 |
| | 5-bromo-2-methoxypyridine (CAS: 13472-85-0) |
| | ¹H NMR (400 MHZ, DMSO-d₆) δ 12.96 (br, 1H), 8.08-7.84 (m, 3H), 7.62 |
| | -7.24 (m, 7H), 6.76-6.64 (m, 1H), 4.85-4.57 (m, 1H), 4.40-4.12 (m, 3H), |
| | 3.87-3.70 (m, 3H), 3.22-2.74 (m, 2H), 2.76-2.62 (m, 3H). LC-MS (ESI, |
| | m/z): [M + H]⁺ 433. |
| 3-28-22 | 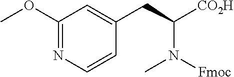 |
| | (2S)-2-({[(9H-fluoren-9-yl)methoxy]carbonyl}(methyl)amino)-3-(2- |
| | methoxypyridin-4-yl)propanoic acid |
| | Reference example-3-1-01 |
| | 4-bromo-2-methoxypyridine (CAS: 100367-39-3) |
| | ¹H NMR (500 MHz, DMSO-d₆) δ 13.07 (br, 1H), 8.09-7.81 (m, 3H), 7.65 |
| | -7.21 (m, 6H), 6.92-6.55 (m, 2H), 4.94-4.62 (m, 1H), 4.39-4.10 (m, 3H), |
| | 3.86-3.70 (m, 3H), 3.25-2.79 (m, 2H), 2.79-2.59 (m, 3H). LC-MS (ESI, |
| | m/z): [M + H]⁺ 433. |
| 3-28-23 | 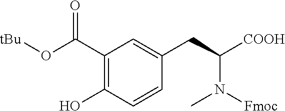 |
| | (2S)-3-{3-[(tert-butoxy)carbonyl]-4-hydroxypheny1}-2-({[(9H-fluoren-9- |
| | yl)methoxy]carbonyl}(methyl)amino)propanoic acid |
| | Reference example-3-1-01 |
| | 2-hydroxy-5-iodobenzoic acid (CAS: 119-30-2) |
| | ¹H NMR (500 MHz, DMSO-d₆) δ 12.98 (br, 1H), 10.58 (s, 1H), 7.93-7.83 |
| | (m, 2H), 7.63-7.55 (m, 1H), 7.55-7.16 (m, 7H), 6.90-6.82 (m, 1H), 4.81 |
| | -4.68 (m, 1H), 4.26-4.06 (m, 3H), 3.21-2.83 (m, 2H), 2.72 (s, 3H), 1.51 - |
| | 1.37 (m, 9H). LC-MS (ESI, m/z): [M + H-tBu]⁺ 462. |
| 3-28-24 | 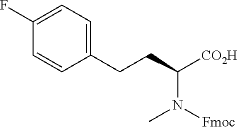 |
| | (2S)-2-({[(9H-fluoren-9-yl)methoxy]carbony1}(methyl)amino)-4-(4- |
| | fluorophenyl)butanoic acid |
| | Reference example-3-1-03 |
| | 1-bromo-4-fluorobenzene (CAS: 460-00-4) |
| | ¹H NMR (400 MHZ, DMSO-d₆) δ 12.87 (br, 1H), 8.04-7.81 (m, 2H), 7.77 |
| | -7.50 (m, 2H), 7.53-7.00 (m, 8H), 4.62-4.10 (m, 4H), 2.94-2.67 (m, 3H), |
| | 2.59-2.29 (m, 2H), 2.24-1.82 (m, 2H). LC-MS (ESI, m/z): [M + H]⁺ 434. |
| 3-28-25 | 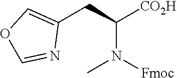 |
| | (2S)-2-({[(9H-fluoren-9-yl)methoxy]carbonyl}(methyl)amino)-3-(1,3- |
| | oxazol-4-yl)propanoic acid |
| | Reference example-3-1-01 |
| | 4-bromo-1,3-oxazole (CAS: 1240598-57-5) |
| | ¹H NMR (400 MHZ, DMSO-d₆) δ 12.98 (br, 1H), 8.38-8.23 (m, 1H), 7.96 |
| | -7.86 (m, 2H), 7.86-7.72 (m, 1H), 7.65-7.55 (m, 2H), 7.48-7.28 (m, 4H), |
| | 4.93-4.75 (m, 1H), 4.37-4.15 (m, 3H), 3.12-2.89 (m, 2H), 2.85-2.74 (m, |
| | 3H). LC-MS (ESI, m/z): [M + H]⁺ 393. |
| 3-28-26 | 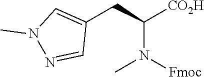 |
| | (2S)-2-({[(9H-fluoren-9-yl)methoxy]carbonyl}(methyl)amino)-3-(1- |
| | methyl-1H-pyrazol-4-yl)propanoic acid |
| | Reference example-3-1-01 |
| | 4-bromo-1-methyl-1H-pyrazole (CAS: 15803-02-8) |
| | ¹H NMR (500 MHZ, DMSO-d₆) δ 12.93 (br, 1H), 7.95-7.84 (m, 2H), 7.62 |
| | -7.47 (m, 2H), 7.46-7.26 (m, 5H), 7.25-7.13 (m, 1H), 4.71-4.53 (m, 1H), |
| | 4.34-4.15 (m, 3H), 3.77-3.66 (m, 3H), 3.04-2.69 (m, 5H). LC-MS (ESI, |
| | m/z): [M + H]⁺ 406. |
| 3-28-27 | 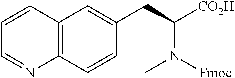 |
| | (2S)-2-({[(9H-fluoren-9-yl)methoxy]carbonyl}(methyl)amino)-3- |
| | (quinolin-6-yl)propanoic acid |
| | Reference example-3-1-01 |
| | 6-bromoquinoline (CAS: 5332-25-2) |
| | ¹H NMR (300 MHz, DMSO-d₆) δ 12.99 (br, 1H), 8.89-8.79 (m, 1H), 8.28 |
| | -8.17 (m, 1H), 7.96-7.77 (m, 4H), 7.71-7.62 (m, 1H), 7.62-7.10 (m, 7H), |
| | 5.01-4.75 (m, 1H), 4.38-4.05 (m, 3H), 3.50-2.94 (m, 2H), 2.79-2.64 (m, |
| | 3H). LC-MS (ESI, m/z): [M + H]⁺ 453. |

### Example-3-29

The following compounds can be synthesized as outlined for the preparation of Reference example-3-3-01 or Example-3-15-01 employing appropriate starting materials in the table.

| | Structure and IUPAC name |
|---|---|
| | Starting material-A |
| | Starting material-B |
| Example | NMR and MS |
| 3-29-01 | 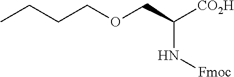 |
| | (2S)-3-butoxy-2-({[(9H-fluoren-9-yl)methoxy]carbonyl}amino)propanoic acid |
| | Reference example-3-2-01 |
| | butan-1-ol (CAS: 71-36-3) |
| | ¹H NMR (400 MHz, DMSO-d₆) δ 12.87 (m, 1H), 7.90 (d, J = 7.5 Hz, 2H), |
| | 7.78-7.71 (m, 2H), 7.59 (d, J = 8.3 Hz, 1H), 7.46-7.39 (m, 2H), 7.38-7.27 |
| | (m, 2H), 4.34-4.15 (m, 4H), 3.70-3.57 (m, 2H), 3.46-3.32 (m, 2H), 1.53- |
| | 1.39 (m, 2H), 1.36-1.23 (m, 2H), 0.90-0.80 (m, 3H). LC-MS (ESI, m/z): |
| | [M − H]⁻ 382. |
| 3-29-02 | 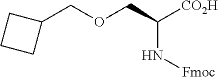 |
| | (2S)-3-(cyclobutylmethoxy)-2-({[(9H-fluoren-9- |
| | yl)methoxy]carbonyl}amino)propanoic acid |
| | Reference example-3-2-01 |
| | cyclobutylmethanol (CAS: 4415-82-1) |
| | ¹H NMR (500 MHz, DMSO-d₆) δ 12.79 (br, 1H), 7.90 (d, J = 7.6 Hz, 2H), |
| | 7.78-7.70 (m, 2H), 7.68-7.58 (m, 1H), 7.47-7.38 (m, 2H), 7.76-7.22 (m, |
| | 2H), 4.37-4.13 (m, 4H), 3.63 (d, J = 5.7 Hz, 2H), 3.56-3.35 (m, 2H), 2.50- |
| | 2.43 (m, 1H), 2.02-1.89 (m, 2H), 1.88-1.74 (m, 2H), 1.72-1.59 (m, 2H). |
| | LC-MS (ESI, m/z): [M + H]⁺ 396. |
| 3-29-03 | 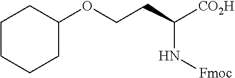 |
| | (2S)-4-(cyclohexyloxy)-2-({[(9H-fluoren-9- |
| | yl)methoxy]carbonyl}amino)butanoic acid |
| | Reference example-3-2-03 |
| | cyclohexanol (CAS: 108-93-0) |
| | ¹H NMR (500 MHz, DMSO-d₆) δ 12.57 (br, 1H), 7.90 (d, J = 7.5 Hz, 2H), |
| | 7.74-7.62 (m, 2H), 7.56 (d, J = 8.2 Hz, 1H), 7.46-7.39 (m, 2H), 7.36-7.28 |
| | (m, 2H), 4.36-4.04 (m, 4H), 3.49-3.38 (m, 2H), 3.27-3.11 (m, 1H), 2.00- |
| | 1.88 (m, 1H), 1.83-1.69 (m, 3H), 1.68-1.54 (m, 2H), 1.51-1.40 (m, 1H), |
| | 1.32-1.06 (m, 5H). LC-MS (ESI, m/z): [M + H]⁺ 424. |
| 3-29-04 | 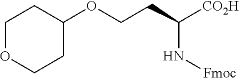 |
| | (2S)-2-({[(9H-fluoren-9-yl)methoxy]carbonyl}amino)-4-(oxan-4- |
| | yloxy)butanoic acid |
| | Reference example-3-2-03 |
| | oxan-4-ol (CAS: 2081-44-9) |
| | ¹H NMR (500 MHz, DMSO-d₆) δ 12.49 (br, 1H), 7.90 (d, J = 7.5 Hz, 2H), |
| | 7.76-7.62 (m, 2H), 7.58 (d, J = 8.1 Hz, 1H), 7.46-7.38 (m, 2H), 7.37-7.28 |
| | (m, 2H), 4.39-4.04 (m, 4H), 3.84-3.67 (m, 2H), 3.54-3.37 (m, 3H), 3.37- |
| | 3.20 (m, 2H), 2.00-1.90 (m, 1H), 1.86-1.70 (m, 3H), 1.45-1.28 (m, 2H). |
| | LC-MS (ESI, m/z): [M + H]⁺ 426. |
| 3-29-05 | 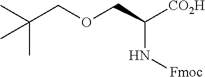 |
| | (2S)-3-(2,2-dimethylpropoxy)-2-({[(9H-fluoren-9- |
| | yl)methoxy]carbonyl}amino)propanoic acid |
| | Reference example-3-2-01 |
| | 2,2-dimethylpropan-1-ol (CAS: 75-84-3) |
| | ¹H NMR (500 MHz, DMSO-d₆) δ 12.76 (br, 1H), 7.89 (d, J = 7.5 Hz, 2H), |
| | 7.77-7.61 (m, 2H), 7.57 (d, J = 8.2 Hz, 1H), 7.45-7.37 (m, 2H), 7.35-7.26 |
| | (m, 2H), 4.36-4.15 (m, 4H), 3.70-3.58 (d, J = 5.8 Hz, 2H), 3.13-3.00 (m, |
| | 2H), 0.85 (s, 9H). LC-MS (ESI, m/z): [M + H]⁺ 398. |
| 3-29-06 | 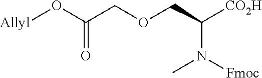 |
| | (2S)-2-({[(9H-fluoren-9-yl)methoxy]carbonyl}(methyl)amino)-3-[2-oxo-2- |
| | (prop-2-en-1-yloxy)ethoxy]propanoic acid |
| | Reference example-3-2-01 |
| | prop-2-en-1-yl 2-hydroxyacetate (CAS: 4704-23-8) |
| | ¹H NMR (500 MHz, DMSO-d₆) δ 13.09 (br, 1H), 8.01-7.81 (m, 2H), 7.71- |
| | 7.56 (m, 2H), 7.50-7.17 (m, 4H), 6.01-5.77 (m, 1H), 5.37-5.27 (m, 1H), |
| | 5.26-5.16 (m, 1H), 4.84-4.72 (m, 1H), 4.67-4.55 (m, 2H), 4.37-4.13 (m, |
| | 5H), 3.97-3.79 (m, 2H), 2.93-2.83 (m, 3H). LC-MS (ESI, m/z): [M + H]⁺ |
| | 440. |

### Example-3-30

The following compounds can be synthesized as outlined for the preparation of Reference example-3-6-01, Example-3-1-01, Example-3-4-01, or Example-3-4-02 employing appropriate starting materials in the table.

| | Structure and IUPAC name |
|---|---|
| | Starting material-A |
| Example | NMR and MS |
| 3-30-01 | 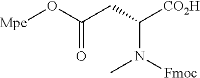 |
| | (2R)-2-({[(9H-fluoren-9-yl)methoxy]carbonyl}(methyl)amino)-4-[(3- |
| | methylpentan-3-yl)oxy]-4-oxobutanoic acid |
| | (2R)-2-{[(benzyloxy)carbonyl]amino}-4-[methylpentan-3-yl)oxy]-4- |
| | oxobutanoic acid |
| | Substantialy identical to Example-3-18-01 |
| 3-30-02 | 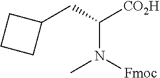 |
| | (R)-2-((((9H-fluoren-9-yl)methoxy)carbonyl)(methyl)amino)-3- |
| | cyclobutylpropanoic acid |
| | (R)-2-((tert-butoxycarbonyl)amino)-3-cyclobutylpropanoic acid (CAS: |
| | 478183-61-8) |
| | ¹H NMR (400 MHz, DMSO-d₆) δ 12.75 (br, 1H), 7.93-7.86 (m, 2H), 7.70- |
| | 7.60 (m, 2H), 7.45-7.38 (m, 2H), 7.37-7.28 (m, 2H), 4.44-4.16 (m, 4H), |
| | 2.68 (s, 3H), 2.16-1.39 (m, 9H). LC-MS (ESI, m/z): [M + H]⁺ 380. |
| 3-30-03 | 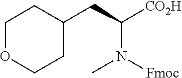 |
| | (2S)-2-({[(9H-fluoren-9-yl)methoxy]carbonyl}(methyl)amino)-3-(oxan-4- |
| | yl)propanoic acid |
| | (2S)-2-({[(9H-fluoren-9-yl)methoxy]carbonyl}amino)-3-(oxan-4- |
| | yl)propanoic acid (CAS: 368866-34-6) |
| | ¹H NMR (500 MHz, DMSO-d₆) δ 12.79 (br, 1H), 7.97-7.82 (m, 2H), 7.71- |
| | 7.57 (m, 2H), 7.49-7.23 (m, 4H), 4.71-4.17 (m, 4H), 3.90-3.64 (m, 2H), |
| | 3.30-3.00 (m, 2H), 2.79-2.61 (m, 3H), 1.79-1.62 (m, 1H), 1.60-1.45 (m, |
| | 2H), 1.42-1.04 (m, 4H). LC-MS (ESI, m/z): [M + H]⁺ 410. |
| 3-30-04 | 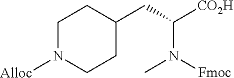 |
| | (2R)-2-({[(9H-fluoren-9-yl)methoxy]carbonyl}(methyl)amino)-3-{1- |
| | [(prop-2-en-1-yloxy)carbonyl]piperidin-4-yl}propanoic acid |
| | (2R)-2-({[(9H-fluoren-9-yl)methoxy]carbonyl}amino)-3-{1-[(prop-2-en-1- |
| | yloxy)carbonyl]piperidin-4-yl}propanoic acid (CAS: 1251903-87-3) |
| | ¹H NMR (500 MHz, DMSO-d₆) δ 12.77 (br, 1H), 7.92-7.86 (m, 2H), 7.67- |
| | 7.59 (m, 2H), 7.45-7.37 (m, 2H), 7.36-7.28 (m, 2H), 5.97-5.88 (m, 1H), |
| | 5.33-5.16 (m, 2H), 4.68-4.18 (m, 6H), 4.00-3.80 (m, 2H), 2.87-2.55 (m, |
| | 5H), 1.78-0.90 (m, 7H). LC-MS (ESI, m/z): [M + H]⁺ 493. |
| 3-30-05 | 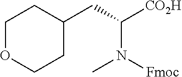 |
| | (2R)-2-({[(9H-fluoren-9-yl)methoxy]carbonyl}(methyl)amino)-3-(oxan-4- |
| | yl)propanoic acid |
| | (2R)-2-({[(9H-fluoren-9-yl)methoxy]carbonyl}amino)-3-(oxan-4- |
| | yl)propanoic acid (CAS: 1879080-16-6) |
| | Substantialy identical to Example-3-30-03 |
| 3-30-06 | 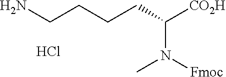 |
| | (2R)-6-amino-2-({[(9H-fluoren-9- |
| | yl)methoxy]carbonyl}(methyl)amino)hexanoic acid hydrochloride |
| | (2R)-6-{[(tert-butoxy)carbonyl]amino}-2-({[(9H-fluoren-9- |
| | yl)methoxy]carbonyl}(methyl)amino)hexanoic acid(CAS: 1793105-27-7) |
| | ¹H NMR (300 MHz, DMSO-d₆) δ 12.83 (br, 1H), 8.26-7.82 (m, 5H), 7.77- |
| | 7.57 (m, 2H), 7.51-7.25 (m, 4H), 4.61-4.20 (m, 4H), 2.89-2.64 (m, 5H), |
| | 1.95-1.43 (m, 4H), 1.35-1.00 (m, 2H). LC-MS (ESI, m/z): [M + H]⁺ 383. |
| 3-30-07 | 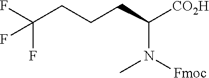 |
| | (2S)-2-({[(9H-fluoren-9-yl)methoxy]carbonyl}(methyl)amino)-6,6,6- |
| | trifluorohexanoic acid |
| | (2S)-2-({[(9H-fluoren-9-yl)methoxy]carbonyl}amino)-6,6,6- |
| | trifluorohexanoic acid (CAS: 2230472-61-2) |
| | ¹H NMR (500 MHz, DMSO-d₆) δ 12.90 (br, 1H), 7.99-7.81 (m, 2H), 7.71- |
| | 7.56 (m, 2H), 7.46-7.37 (m, 2H), 7.37-7.26 (m, 2H), 4.59-4.21 (m, 4H), |
| | 2.77-2.67 (m, 3H), 2.35-2.13 (m, 2H), 1.98-1.55 (m, 2H), 1.46-1.20 (m, |
| | 2H). LC-MS (ESI, m/z): [M + H]⁺ 422. |
| 3-30-08 | 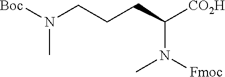 |
| | (2S)-5-{[(tert-butoxy)carbonyl](methyl)amino}-2-({[(9H-fluoren-9- |
| | yl)methoxy]carbonyl}(methyl)amino)pentanoic acid |
| | (2S)-2-{[(benzyloxy)carbonyl]amino}-5-{[(tert- |
| | butoxy)carbonyl]amino}pentanoic acid (CAS: 7733-29-1) |
| | ¹H NMR (500 MHz, DMSO-d₆) δ 12.81 (br, 1H), 8.02-7.79 (m, 2H), 7.71- |
| | 7.53 (m, 2H), 7.47-7.21 (m, 4H), 4.60-4.11 (m, 4H), 3.28-3.00 (m, 2H), |
| | 2.82-2.62 (m, 6H), 1.87-1.47 (m, 2H), 1.48-1.07 (m, 11H). LC-MS (ESI, |
| | m/z): [M + H]⁺ 483. |
| 3-30-09 | 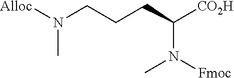 |
| | (2S)-2-({[(9H-fluoren-9-yl)methoxy]carbonyl}(methyl)amino)-5- |
| | {methyl[(prop-2-en-1-yloxy)carbonyl]amino}pentanoic acid |
| | (2S)-2-{[(tert-butoxy)carbonyl]amino}-5-{[(prop-2-en-1- |
| | yloxy)carbonyl]amino}pentanoic acid (CAS: 171820-74-9) |
| | ¹H NMR (500 MHz, DMSO-d₆) δ 12.85 (br, 1H), 7.99-7.80 (m, 2H), 7.69- |
| | 7.57 (m, 2H), 7.46-7.36 (m, 2H), 7.35-7.25 (m, 2H), 5.99-5.84 (m, 1H), |
| | 5.33-5.06 (m, 2H), 4.57-4.19 (m, 6H), 3.29-3.11 (m, 2H), 2.88-2.76 (m, |
| | 3H), 2.75-2.65 (m, 3H), 1.84-1.47 (m, 2H), 1.44-1.21 (m, 2H). LC-MS |
| | (ESI, m/z): [M + H]⁺ 467. |
| 3-30-10 | 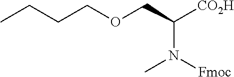 |
| | (2S)-3-butoxy-2-({[(9H-fluoren-9- |
| | yl)methoxy]carbonyl}(methyl)amino)propanoic acid |
| | (2S)-3-butoxy-2-({[(9H-fluoren-9-yl)methoxy]carbonyl}amino)propanoic |
| | acid (CAS: 133368-40-8) |
| | ¹H NMR (300 MHz, DMSO-d₆) δ 12.92 (br, 1H), 7.99-7.83 (m, 2H), 7.75- |
| | 7.58 (m, 2H), 7.51-7.25 (m, 4H), 4.83-4.64 (m, 1H), 4.43-4.19 (m, 3H), |
| | 3.88-3.62 (m, 2H), 3.60-3.06 (m, 2H), 2.95-2.76 (m, 3H), 1.53-1.38 (m, |
| | 2H), 1.36-1.18 (m, 2H), 0.92-0.75 (m, 3H). LC-MS (ESI, m/z): [M + H]⁺ 398. |
| 3-30-11 | 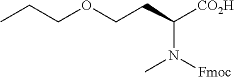 |
| | (2S)-2-({[(9H-fluoren-9-yl)methoxy]carbonyl}(methyl)amino)-4- |
| | propoxybutanoic acid |
| | (2S)-2-({[(9H-fluoren-9-yl)methoxy]carbonyl}amino)-4-propoxybutanoic |
| | acid (CAS: 2349638-26-0) |
| | ¹H NMR (400 MHz, DMSO-d₆) δ 12.89 (br, 1H), 7.97-7.82 (m, 2H), 7.75- |
| | 7.58 (m, 2H), 7.49-7.26 (m, 4H), 4.66-4.52 (m, 1H), 4.42-4.16 (m, 3H), |
| | 3.45-3.15 (m, 4H), 2.78 (s, 3H), 2.17-2.01 (m, 1H), 2.01-1.77 (m, 1H), |
| | 1.55-1.39 (m, 2H), 0.89-0.78 (m, 3H). LC-MS (ESI, m/z): [M + H]⁺ 398. |
| 3-30-12 | 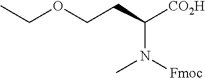 |
| | (2S)-4-ethoxy-2-({[(9H-fluoren-9- |
| | yl)methoxy]carbonyl}(methyl)amino)butanoic acid |
| | (2S)-4-ethoxy-2-({[(9H-fluoren-9-yl)methoxy]carbonyl}amino)butanoic |
| | acid (CAS: 1932074-99-1) |
| | ¹H NMR (400 MHz, DMSO-d₆) δ 12.82 (br, 1H), 7.96-7.86 (m, 2H), 7.71- |
| | 7.60 (m, 2H), 7.48-7.27 (m, 4H), 4.62-4.50 (m, 1H), 4.41-4.18 (m, 3H), |
| | 3.49-3.13 (m, 4H), 2.76 (s, 3H), 2.13-1.98 (m, 1H), 1.97-1.77 (m, 1H), |
| | 1.11-1.03 (m, 3H). LC-MS (ESI, m/z): [M + H]⁺ 384. |

### Example-3-31-01: (2S,4R)-1-(((9H-fluoren-9-yl)methoxy)carbonyl)-4-(pyridin-3-yloxy)pyrrolidine-2-carboxylic acid

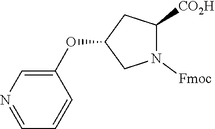

To a suspension of 1-(tert-butyl) 2-methyl (2S,4S)-4-hydroxypyrrolidine-1,2-dicarboxylate (CAS: 102195-79-9, 1.23 g, 5.00 mmol) in THF (10.0 mL) at 0° C. was added 3-Hydroxypyridine (CAS: 109-00-2, 0.52 g, 5.50 mmol), triphenylphosphane (1.57 g, 6.00 mmol), and diisopropyl azodicarboxylate (CAS: 2446-83-5, 3.29 mL, 1.9M in toluene). The mixture was stirred at rt for 18 h, and then concentrated. The resulting residue was purified by silica gel flash column chromatography (gradient, hexane/EtOAc=90/10 to 0/100). Appropriate fractions were concentrated. To the solution of resulting residue in THF (20.0 mL) and MeOH (10.0 mL) at 0° C. was added 1M aq. sodium hydroxide (10.0 mL, 10.0 mmol). The mixture was stirred at rt for 1 h. The solution was diluted with EtOAc and organic layer was extracted twice with 1M aq. sodium hydroxide. The combined aqueous layer was acidified with 1M aq. HCl, extracted twice with EtOAc. The combined organic extracts were washed with brine, dried over Na₂SO₄, filtered, and then concentrated. To the solution of resulting residue in DCM (15.0 mL) at rt was added 4M hydrogen chloride in CPME (18.8 mL, 75.2 mmol). The mixture was stirred at rt for 2 h, and then concentrated. To a solution of the resulting residue in 1,4-dioxane (10.0 mL) and H₂O (10.0 mL) at 0° C. was added NaHCO₃ (1.77 g, 21.0 mmol), followed by a solution of Fmoc-OSu (2.36 g, 7.01 mmol) in 1,4-dioxane (10.0 mL) dropwise. The mixture was stirred at rt for 18 h. The mixture was acidified with 1M aq. HCl. The mixture was extracted twice with EtOAc. The combined organic extracts were washed with brine, dried over Na₂SO₄, filtered, and then concentrated. The resulting residue was purified by silica gel flash column chromatography (gradient, hexane/EtOAc=90/10 to 0/100) to afford the title compound (1.76 g, 4.09 mmol). ¹H NMR (500 MHz, DMSO-d₆) δ 12.90 (br, 1H), 8.38-7.80 (m, 3H), 7.71-7.51 (m, 3H), 7.48-7.30 (m, 4H), 7.28-7.15 (m, 1H), 4.54-4.05 (m, 5H), 3.77-3.61 (m, 2H), 3.55-3.43 (m, 1H), 2.65-2.52 (m, 1H), 2.42-2.17 (m, 1H). LC-MS (ESI, m/z): [M+H]⁺ 431.

### Example-3-31

The following compounds can be synthesized as outlined for the preparation of Example-3-21-01, Example-3-21-02, or Example-3-31-01 employing appropriate starting materials in the table.

| | Structure and IUPAC name |
|---|---|
| | Starting material-A |
| Example | NMR and MS |
| 3-31-02 | 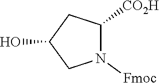 |
| | (2R,4R)-1-{[(9H-fluoren-9-yl)methoxy]carbonyl}-4-hydroxypyrrolidine-2- |
| | carboxylic acid |
| | 1-(tert-butyl) 2-methyl (2R,4S)-4-hydroxypyrrolidine-1,2-dicarboxylate |
| | (CAS: 135042-17-0) |
| | ¹H NMR (500 MHz, DMSO-d₆) δ 12.51 (br, 1H), 7.93-7.86 (m, 2H), 7.70- |
| | 7.64 (m, 2H), 7.46-7.29 (m, 4H), 5.01(br, 1H), 4.38-4.15 (m, 5H), 3.59- |
| | 3.49 (m, 1H), 3.23-3.16 (m, 1H), 2.42-2.26 (m, 1H), 2.02-1.84 (m, 1H). |
| | LC-MS (ESI, m/z): [M + H]⁺ 354. |
| 3-31-03 | 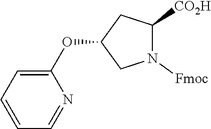 |
| | (2S,4R)-1-(((9H-fluoren-9-yl)methoxy)carbonyl)-4-(pyridin-2- |
| | yloxy)pyrrolidine-2-carboxylic acid |
| | 1-(tert-butyl) 2-methyl (2S,4S)-4-hydroxypyrrolidine-1,2-dicarboxylate |
| | (CAS: 102195-79-9) |
| | ¹H NMR (500 MHz, DMSO-d₆) δ 12.84 (br, 1H), 8.25-8.15 (m, 1H), 7.90 |
| | (d, J = 7.5 Hz, 1H), 7.87-7.80 (m, 1H), 7.79-7.71 (m, 1H), 7.70-7.63 (m, |
| | 1H), 7.60-7.51 (m, 1H), 7.46-7.29 (m, 3H), 7.28-7.16 (m, 1H), 7.08- |
| | 6.96 (m, 1H), 6.89-6.80 (m, 1H), 4.54-4.15 (m, 4H), 3.84-3.73 (m, 1H), |
| | 3.71-3.60 (m, 1H), 2.64-2.55 (m, 1H), 2.49-2.46 (m, 1H), 2.44-2.20 (m, |
| | 1H). LC-MS (ESI, m/z): [M + H]⁺ 431. |

### Example-3-32: (S)-2-((((9H-fluoren-9-yl)methoxy)carbonyl)amino)-4-(dimethylamino)butanoic acid hydrochloride

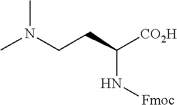

The title compound can be synthesized as outlined for the preparation methods described in Chemical Communications (2015), 51(21), 4496. employing appropriate starting material (2S)-4-{[(tert-butoxy)carbonyl]amino}-2-({[(9H-fluoren-9-yl)methoxy]carbonyl}amino)butanoic acid (CAS: 125238-99-5). ¹H NMR (500 MHz, DMSO-d₆) δ 12.95 (br, 1H), 10.52 (s, 1H), 7.95-7.87 (m, 2H), 7.83-7.75 (m, 1H), 7.76-7.67 (m, 2H), 7.48-7.30 (m, 4H), 4.43-4.14 (m, 3H), 4.09-3.99 (m, 1H), 3.21-3.09 (m, 1H), 3.08-2.94 (m, 1H), 2.78-2.67 (m, 6H), 2.23-2.11 (m, 1H), 2.07-1.92 (m, 1H). LC-MS (ESI, m/z): [M+H]⁺ 369.

### Example-3-33: (S,Z)-2-((((9H-fluoren-9-yl)methoxy)carbonyl)amino)-3-(4-(2-methyl-3-((2,2,4,6,7-pentamethyl-2,3-dihydrobenzofuran-5-yl)sulfonyl)guanidino)phenyl)propanoic acid

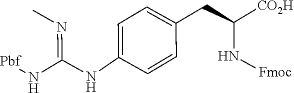

To a solution of 2,2,4,6,7-pentamethyl-2,3-dihydrobenzofuran-5-sulfonamide (CAS: 378230-81-0, 1.70 g, 6.31 mmol) in DMF (25.0 mL) at 0° C. was added sodium hydride (0.27 g, 60% dispersion in mineral oil, 6.63 mmol). The mixture was stirred at rt for 15 min and then isothiocyanatomethane (CAS: 556-61-6, 0.46 g, 6.31 mmol) was added and stirred at 50° C. for 30 min. After cooled to rt, reaction mixture was diluted with water. The mixture was acidified with 2M aq. HCl. The aqueous phase was extracted with hexane/ethyl acetate (1/1). The combined organic extracts were washed four times with water, brine, dried over Na₂SO₄, filtered, and then concentrated. To a solution of the obtained compound (2.20 g, 6.42 mmol) and (2S)-3-(4-aminophenyl)-2-({[(9H-fluoren-9-yl)methoxy]carbonyl}amino)propanoic acid (CAS: 95753-56-3, 2.43 g, 5.84 mmol) in DMF (30.0 mL) at rt was added EDCI·HCl. The resulting mixture was stirred at rt for 1 h. The reaction mixture at 0° C. was quenched with satd. aq. NaHCO₃. The aqueous phase was extracted with hexane/ethyl acetate (1/1). The combined organic extracts were washed four times with H₂O, brine, dried over Na₂SO₄, filtered, and then concentrated. The resulting residue was purified by silica gel flash column chromatography (gradient, hexane/EtOAc=100/0 to 30/70). Appropriate fractions were concentrated. To a solution of the resulting residue (4.50 g, 6.21 mmol) in ClCH₂CH₂Cl (35.0 mL) at rt was added Me₃SnOH (4.49 g, 24.8 mmol). The resulting mixture was stirred at 50° C. for 6 h. The reaction mixture was concentrated. The resulting residue was purified by silica gel flash column chromatography (gradient, CH₂Cl₂/MeOH=100/0 to 90/10) to afford the title compound (3.75 g, 5.28 mmol). ¹H NMR (500 MHz, DMSO-d₆) 8.91 (br, 1H), 7.82-7.70 (m, 2H), 7.60-7.49 (m, 2H), 7.46-7.34 (m, 2H), 7.36-7.23 (m, 2H), 7.20-7.07 (m, 2H), 7.11-6.97 (m, 2H), 6.95 (br, 1H), 5.35 (br, 1H), 4.72-4.57 (m, 1H), 4.53-4.42 (m, 1H), 4.41-4.32 (m, 1H), 4.25-4.15 (m, 1H), 3.25-3.03 (m, 2H), 2.97 (s, 2H), 2.80-2.70 (m, 3H), 2.66 (s, 3H), 2.57 (s, 3H), 2.12 (s, 3H), 1.47 (s, 6H). LC-MS (ESI, m/z): [M+H]⁺ 711.

### Example-3-34: (S)-2-((((9H-fluoren-9-yl)methoxy)carbonyl)amino)-3-(4-((1-((2,2,4,6,7-pentamethyl-2,3-dihydrobenzofuran-5-yl)sulfonyl)-4,5-dihydro-1H-imidazol-2-yl)amino)phenyl)propanoic acid

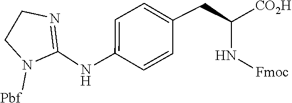

To a suspension of 2-(methylthio)-4,5-dihydro-1H-imidazole hydroiodide (CAS: 5464-11-9, 2.00 g, 8.19 mmol) in DCM (20.5 mL) at 0° C. was added triethylamine (2.28 mL, 16.4 mmol) and 2,2,4,6,7-Pentamethyl-2,3-dihydrobenzofuran-5-sulfonyl chloride (CAS: 154445-78-0, 2.96 g, 10.2 mmol) in DCM (20.5 mL). The mixture was stirred at rt for 15 h. The reaction mixture at 0° C. was quenched with H₂O. The aqueous phase was extracted with DCM. The combined organic extracts were washed twice with 1M aq. NaHCO₃, brine, dried over Na₂SO₄, filtered, and then concentrated. The resulting residue was purified by silica gel flash column chromatography (gradient, hexane/EtOAc=100/0 to 0/100). A mixture of the obtained compound (2.12 g, 5.75 mmol) and (2S)-3-(4-aminophenyl)-2-({[(9H-fluoren-9-yl)methoxy]carbonyl}amino)propanoic acid (CAS: 95753-56-3, 2.01 g, 5.00 mmol) in AcOH (12.5 mL) was stirred at 80° C. for 18 h and then stirred at 100° C. for 8 h. The mixture was concentrated. The residue was azeotroped with toluene five times. The resulting residue was purified by silica gel flash column chromatography (gradient, CH₂Cl₂/MeOH=95/5 to 80/20), and then silica gel flash column chromatography (gradient, CH₂Cl₂/MeOH=100/0 to 85/15) to afford the title compound (2.14 g, 2.96 mmol). ¹H NMR (500 MHz, DMSO-d₆) δ 12.98 (br, 1H), 7.88 (d, J=7.6 Hz, 2H), 7.74-7.47 (m, 3H), 7.45-7.36 (m, 2H), 7.34-7.23 (m, 2H), 7.21-7.00 (m, 2H), 6.77-6.34 (m, 3H), 4.29-4.04 (m, 4H), 4.01-3.81 (m, 2H), 3.54-3.25 (m, 2H), 3.05-2.64 (m, 4H), 2.49-2.36 (m, 6H), 2.03 (s, 3H), 1.39 (s, 6H). LC-MS (ESI, m/z): [M+H]⁺ 723.

### Example-3-35

The following compounds can be synthesized as outlined for the preparation of Reference example-3-1-04, Example-3-2-06, Example-3-3-05, or Example-3-7-04 employing appropriate starting materials in the table.

| | Structure and IUPAC name |
|---|---|
| | Starting material-A |
| | Starting material-B |
| Example | NMR and MS |
| 3-35-01 | 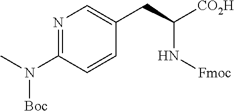 |
| | (2S)-3-(6-{[(tert-butoxy)carbonyl](methyl)amino}pyridin-3-yl)-2-({[(9H- |
| | fluoren-9-yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | tert-butyl N-(5-bromopyridin-2-yl)-N-methylcarbamate (CAS: 227939-01-7) |
| | ¹H NMR (500 MHz, DMSO-d₆) δ 12.94 (br, 1H), 8.24 (s, 1H), 7.88 (d, J = |
| | 7.5 Hz, 2H), 7.76 (d, J = 8.5 Hz, 1H) 7.69-7.25 (m, 8H), 4.34-3.98 (m, |
| | 4H), 3.28-3.19 (m, 3H), 3.15-3.00 (m, 1H), 3.00-2.81(m, 1H),1.43 (s, |
| | 9H). LC-MS (ESI, m/z): [M + H]⁺ 518. |
| 3-35-02 | 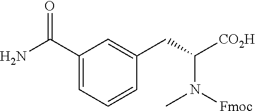 |
| | (2R)-3-(3-carbamoylphenyl)-2-({[(9H-fluoren-9- |
| | yl)methoxy]carbonyl}(methyl)amino)propanoic acid |
| | Reference example-3-1-02 |
| | 3-bromobenzamide (CAS: 698-67-9) |
| | ¹H NMR (500 MHz, DMSO-d₆) δ 12.66 (br, 1H), 7.98-7.77 (m, 4H), 7.77- |
| | 7.67 (m, 1H), 7.59-7.14 (m, 9H), 4.88-4.79 (m, 1H), 4.26-4.09 (m, 3H), |
| | 3.30-2.91 (m, 2H), 2.74-2.69 (m, 3H). LC-MS (ESI, m/z): [M + H]⁺ 445. |
| 3-35-03 | 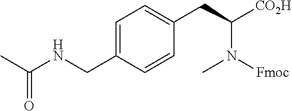 |
| | (2S)-3-[4-(acetamidomethyl)phenyl]-2-({[(9H-fluoren-9- |
| | yl)methoxy]carbonyl}(methyl)amino)propanoic acid |
| | Reference example-3-1-01 |
| | N-[(4-bromophenyl)methyl]acetamide (CAS: 90561-76-5) |
| | ¹H NMR (500 MHz, DMSO-d₆) δ 12.92 (br, 1H), 8.32-8.25 (m, 1H), 7.89 |
| | (d, J = 7.5 Hz, 2H), 7.61-7.49 (m, 2H), 7.46-7.38 (m, 2H), 7.36-7.27 (m, |
| | 2H), 7.18-6.96 (m, 4H), 4.80-4.62 (m, 1H), 4.36-4.13 (m, 5H), 3.22- |
| | 2.77 (m, 2H), 2.74-2.62 (m, 3H), 1.89-1.80 (m, 3H). LC-MS (ESI, m/z): |
| | [M + H]⁺ 473. |
| 3-35-04 | 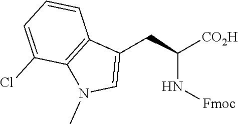 |
| | (2S)-3-(7-chloro-1-methyl-1H-indol-3-yl)-2-({[(9H-fluoren-9- |
| | yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | 3-Bromo-7-chloro-1-methyl-1H-indole (CAS: 2114167-81-4) |
| | ¹H NMR (500 MHz, DMSO-d₆) δ 12.75 (br, 1H), 7.95-7.82 (m, 2H), 7.77- |
| | 7.09 (m, 10H), 7.05-6.94 (m, 1H), 4.29-3.96 (m, 7H), 3.21-3.09 (m, |
| | 1H), 3.06-2.87 (m, 1H). LC-MS (ESI, m/z): [M + H]⁺ 475. |
| 3-35-05 | 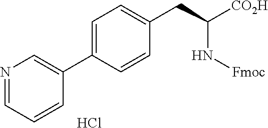 |
| | (2S)-2-({[(9H-fluoren-9-yl)methoxy]carbonyl}amino)-3-[4-(pyridin-3- |
| | yl)phenyl]propanoic acid hydrochloride |
| | methyl (2R)-2-({[(9H-fluoren-9-yl)methoxy]carbonyl}amino)-3- |
| | iodopropanoate (CAS: 156017-42-4) |
| | 3-(4-Bromophenyl)pyridine (CAS: 129013-83-8) |
| | ¹H NMR (500 MHz, DMSO-d₆) δ 13.47-12.04 (m, 2H), 9.12-9.00 (m, |
| | 1H), 8.78-8.68 (m, 1H), 8.52-8.35 (m, 1H), 7.96-6.98 (m, 14H), 4.36- |
| | 4.09 (m, 4H), 3.27-3.08 (m, 1H), 3.03-2.79 (m, 1H). LC-MS (ESI, m/z): |
| | [M + H]⁺ 465. |
| 3-35-06 | 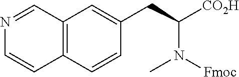 |
| | (2S)-2-({[(9H-fluoren-9-yl)methoxy]carbonyl}(methyl)amino)-3- |
| | (isoquinolin-7-yl)propanoic acid |
| | Reference example-3-1-01 |
| | 7-Bromoisoquinoline (CAS: 58794-09-5) |
| | ¹H NMR (400 MHz, DMSO-d₆) δ 13.78-12.27 (m, 2H), 9.19 (d, J = 13.2 |
| | Hz, 1H), 8.52-8.40 (m, 1H), 8.00-7.09 (m, 12H), 5.07-4.77 (m, 1H), 4.39- |
| | 4.07 (m, 3H), 3.69-3.01 (m, 2H), 2.81-2.67 (m, 3H). LC-MS (ESI, m/z): |
| | [M + H]⁺ 453. |
| 3-35-07 | 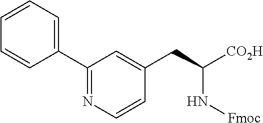 |
| | (2S)-2-({[(9H-fluoren-9-yl)methoxy]carbonyl}amino)-3-(2-phenylpyridin- |
| | 4-yl)propanoic acid |
| | methyl (2R)-2-({[(9H-fluoren-9-yl)methoxy]carbonyl}amino)-3- |
| | iodopropanoate (CAS: 156017-42-4) |
| | 4-Bromo-2-phenylpyridine (CAS: 98420-98-5) |
| | ¹H NMR (500 MHz, DMSO-d₆) δ 12.88 (br, 1H), 8.59-8.52 (m, 1H), 8.07 |
| | (d, J = 7.2 Hz, 2H), 7.93-7.80 (m, 4H), 7.64-7.33 (m, 7H), 7.28-7.06 (m, |
| | 3H), 4.37-4.30 (m, 1H), 4.24-4.11 (m, 3H), 3.24-3.10 (m, 1H), 3.01- |
| | 2.80 (m, 1H). LC-MS (ESI, m/z): [M + H]⁺ 465. |
| 3-35-08 | 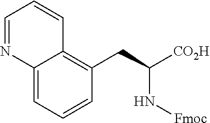 |
| | (2S)-2-({[(9H-fluoren-9-yl)methoxy]carbonyl}amino)-3-(quinolin-5- |
| | yl)propanoic acid |
| | methyl (2R)-2-({[(9H-fluoren-9-yl)methoxy]carbonyl}amino)-3- |
| | iodopropanoate (CAS: 156017-42-4) |
| | 5-Bromoquinoline (CAS: 4964-71-0) |
| | ¹H NMR (500 MHz, DMSO-d₆) δ 12.97 (br, 1H), 8.97-8.87 (m, 1H), 8.56 |
| | (d, J = 8.6 Hz, 1H), 7.99-7.07 (m, 13H), 4.36-3.90 (m, 4H), 3.70-3.12 |
| | (m, 2H). LC-MS (ESI, m/z): [M + H]⁺ 439. |
| 3-35-09 | 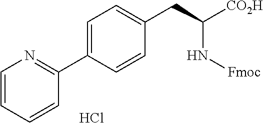 |
| | (2S)-2-({[(9H-fluoren-9-yl)methoxy]carbonyl}amino)-3-[4-(pyridin-2- |
| | yl)phenyl]propanoic acid hydrochloride |
| | methyl (2R)-2-({[(9H-fluoren-9-yl)methoxy]carbonyl}amino)-3- |
| | iodopropanoate (CAS: 156017-42-4) |
| | 2-(4-Bromophenyl) pyridine (CAS: 63996-36-1) |
| | ¹H NMR (500 MHz, DMSO-d₆) δ 13.45 (br, 1H), 8.65 (s, 1H), 7.96 (d, J = |
| | 8.0 Hz, 2H), 7.94-7.81 (m, 4H), 7.68-7.61 (m, 2H), 7.42-7.21 (m, 8H), |
| | 4.26-3.95 (m, 4H), 3.18-3.10 (m, 1H), 3.00-2.90 (m, 1H). LC-MS (ESI, |
| | m/z): [M + H]⁺ 465. |
| 3-35-10 | 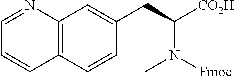 |
| | (2S)-2-({[(9H-fluoren-9-yl)methoxy]carbonyl}(methyl)amino)-3- |
| | (quinolin-7-yl)propanoic acid |
| | Reference example-3-1-01 |
| | 7-Bromoquinoline (CAS: 4965-36-0) |
| | ¹H NMR (400 MHz, DMSO-d₆) δ 13.11 (br, 1H), 9.21-9.06 (m, 1H), 9.06- |
| | 8.88 (m, 1H), 8.28-8.08 (m, 2H), 7.96-7.10 (m, 10H), 5.11-4.85 (m, |
| | 1H), 4.38-4.07 (m, 3H), 3.60-3.15 (m, 2H), 2.81-2.65 (m, 3H). LC-MS |
| | (ESI, m/z): [M + H]⁺ 453. |
| 3-35-11 | 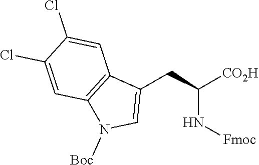 |
| | (2S)-3-{1-[(tert-butoxy)carbonyl]-5,6-dichloro-1H-indol-3-yl}-2-({[(9H- |
| | fluoren-9-yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-5,6-dichloro-1H-indole-1-carboxylate (CAS: 2089314- |
| | 83-8) |
| | ¹H NMR (400 MHz, DMSO-d₆) δ 13.01 (br, 1H), 8.16 (s, 1H), 7.98 (s, 1H), |
| | 7.86 (d, J = 7.6 Hz, 2H), 7.76-7.52 (m, 4H), 7.46-7.30 (m, 2H), 7.30-7.11 |
| | (m, 2H), 4.31-4.11 (m, 4H), 3.61-2.98 (m, 2H), 1.71-1.49 (m, 9H). LC- |
| | MS (ESI, m/z): [M − H]⁻ 593. |
| 3-35-12 | 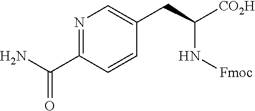 |
| | (2S)-3-(6-carbamoylpyridin-3-yl)-2-({[(9H-fluoren-9- |
| | yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | 5-Bromo-2-pyridinecarboxamide (CAS: 90145-48-5) |
| | ¹H NMR (300 MHz, DMSO-d₆) δ 12.84 (br, 1H), 8.53 (br, 1H), 8.07 (br, |
| | 1H), 8.02-7.92 (m, 1H), 7.92-7.70 (m, 4H), 7.70-7.50 (m, 3H), 7.46-7.35 |
| | (m, 2H), 7.35-7.21 (m, 2H), 4.40-4.09 (m, 4H), 3.25-3.09 (m, 1H), 3.09- |
| | 2.89 (m, 1H). LC-MS (ESI, m/z): [M + H]⁺ 432. |
| 3-35-13 | 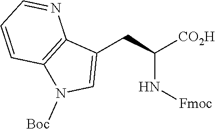 |
| | (2S)-3-{1-[(tert-butoxy)carbonyl]-1H-pyrrolo[3,2-b]pyridin-3-yl}-2- |
| | ({[(9H-fluoren-9-yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-bromo-1H-pyrrolo[3,2-b]pyridine-1-carboxylate (CAS: |
| | 192189-15-4) |
| | ¹H NMR (500 MHz, DMSO-d₆) δ 12.77 (br, 1H), 8.58-8.48 (m, 1H), 8.34- |
| | 8.28 (m 1H), 8.02-7.80 (m, 4H), 7.65-7.47 (m, 2H), 7.47-7.05 (m, 5H), |
| | 4.69-4.40 (m, 1H), 4.38-3.96 (m, 3H), 3.51-3.26 (m, 1H), 3.14-2.95 (m, |
| | 1H), 1.66-1.55 (m, 9H). LC-MS (ESI, m/z): [M − Boc + H]⁺ 428. |
| 3-35-14 | 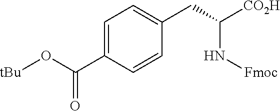 |
| | (2R)-3-{4-[(tert-butoxy)carbonyl]phenyl}-2-({[(9H-fluoren-9- |
| | yl)methoxy]carbonyl}amino)propanoic acid |
| | methyl (2S)-2-({[(9H-fluoren-9-yl)methoxy]carbonyl}amino)-3- |
| | iodopropanoate (CAS: 527696-77-1) |
| | tert-Butyl 4-bromobenzoate (CAS: 59247-47-1) |
| | ¹H NMR (400 MHz, DMSO-d₆) δ 12.96 (br, 1H), 7.88 (d, J = 7.6 Hz, 2H), |
| | 7.85-7.76 (m, 2H), 7.72 (d, J = 8.6 Hz, 1H), 7.64-7.58 (m, 2H), 7.45-7.20 |
| | (m, 6H), 4.27-4.10 (m, 4H), 3.21-3.13 (m, 1H), 3.00-2.86 m, 1H), 1.52 |
| | (s, 9H). LC-MS (ESI, m/z): [M − H]⁻ 486. |
| 3-35-15 | 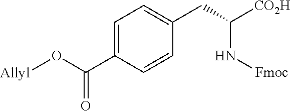 |
| | (2R)-2-({[(9H-fluoren-9-yl)methoxy]carbonyl}amino)-3-{4-[(prop-2-en-1- |
| | yloxy)carbonyl]phenyl}propanoic acid |
| | methyl (2S)-2-({[(9H-fluoren-9-yl)methoxy]carbonyl}amino)-3- |
| | iodopropanoate (CAS: 527696-77-1) |
| | prop-2-en-1-yl 4-bromobenzoate (CAS: 6420-77-5) |
| | ¹H NMR (400 MHz, DMSO-d₆) δ 12.87 (br, 1H), 7.95-7.84 (m, 4H), 7.79 |
| | (d, J = 8.4 Hz, 1H), 7.65-7.57 (m, 2H), 7.57-7.18 (m, 6H), 6.10-5.93 (m, |
| | 1H), 5.45-5.33 (m, 1H), 5.32-5.20 (m, 1H), 4.79 (d, J = 5.4 Hz, 2H), 4.30- |
| | 4.12 (m, 4H), 3.25-3.09 (m, 1H), 3.05-2.80 (m, 1H). LC-MS (ESI, |
| | m/z): [M + NH₄]⁺ 489. |
| 3-35-16 | 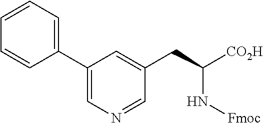 |
| | (S)-2-((((9H-fluoren-9-yl)methoxy)carbonyl)amino)-3-(5-phenylpyridin-3- |
| | yl)propanoic acid |
| | methyl (2R)-2-({[(9H-fluoren-9-yl)methoxy]carbonyl}amino)-3- |
| | iodopropanoate (CAS: 156017-42-4) |
| | 3-bromo-5-phenylpyridine (CAS: 142137-17-5) |
| | ¹H NMR (500 MHz, DMSO-d₆) δ 12.9 (br, 1H), 8.81-8.73 (m, 1H), 8.53- |
| | 8.46 (m, 1H), 8.08-7.99 (m, 1H), 7.95-7.81 (m, 3H), 7.78-7.68 (m, 2H), |
| | 7.67-7.32 (m, 7H), 7.30-7.16 (m, 2H), 4.38-4.26 (m, 1H), 4.23-4.09 (m, |
| | 3H), 3.27-3.12 (m, 1H), 3.04-2.81 (m, 1H). LC-MS (ESI, m/z): [M + H]⁺ |
| | 465. |
| 3-35-17 | 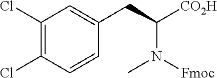 |
| | (S)-2-((((9H-fluoren-9-yl)methoxy)carbonyl)(methyl)amino)-3-(3,4- |
| | dichlorophenyl)propanoic acid |
| | Reference example-3-1-01 |
| | 4-bromo-1,2-dichlorobenzene (CAS: 18282-59-2) |
| | ¹H NMR (500 MHz, DMSO-d₆) δ 13.0 (br, 1H), 7.96-7.81 (m, 2H), 7.63- |
| | 6.95 (m, 9H), 4.93-4.64 (m, 1H), 4.41-4.12 (m, 3H), 3.28-2.77 (m, 2H), |
| | 2.73-2.64 (m, 3H). LC-MS (ESI, m/z): [M + H]⁺ 470. |
| 3-35-18 | 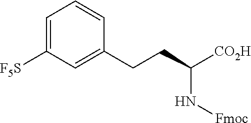 |
| | (S)-2-((((9H-fluoren-9-yl)methoxy)carbonyl)amino)-4-(3-(pentafluoro-l6- |
| | sulfaneyl)phenyl)butanoic acid |
| | Reference example-3-1-03 |
| | (3-bromophenyl)pentafluoro-l6-sulfane (CAS: 672-30-0) |
| | ¹H NMR (400 MHz, DMSO-d₆) δ 12.67 (br, 1H), 7.90 (d, J = 7.6 Hz, 2H), |
| | 7.79-7.67 (m, 5H), 7.62-7.48 (m, 2H), 7.45-7.39 (m, 2H), 7.37-7.30 (m, |
| | 2H), 4.37-4.15 (m, 3H), 3.94-3.86 (m, 1H), 2.81-2.67 (m, 2H), 2.08- |
| | 1.84 (m, 2H). LC-MS (ESI, m/z): [M − H]⁻ 526. |
| 3-35-19 | 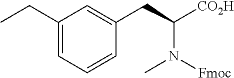 |
| | (2S)-3-(3-ethylphenyl)-2-({[(9H-fluoren-9- |
| | yl)methoxy]carbonyl}(methyl)amino)propanoic acid |
| | Reference example-3-1-01 |
| | 1-bromo-3-ethylbenzene (CAS: 2725-82-8) |
| | ¹H NMR (400 MHz, DMSO-d₆) δ 12.18 (br, 1H), 7.95-7.85 (m, 2H), 7.63- |
| | 7.37 (m, 4H), 7.36-7.24 (m, 2H), 7.22-7.12 (m, 1H), 7.11-6.85 (m, 3H), |
| | 4.87-4.70 (m, 1H), 4.36-4.09 (m, 3H), 3.25-2.79 (m, 2H), 2.76-2.64 (m, |
| | 3H), 2.59-2.46 (m, 2H), 1.15-1.06 (m, 3H). LC-MS (ESI, m/z): [M + H]⁺ |
| | 430. |
| 3-35-20 | 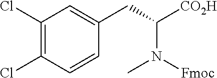 |
| | (2R)-3-(3,4-dichlorophenyl)-2-({[(9H-fluoren-9- |
| | yl)methoxy]carbonyl}(methyl)amino)propanoic acid |
| | Reference example-3-1-02 |
| | 4-bromo-1,2-dichlorobenzene (CAS: 18282-59-2) |
| | Substantialy identical to Example-3-35-17 |
| 3-35-21 | 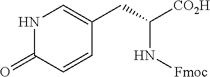 |
| | (2R)-2-({[(9H-fluoren-9-yl)methoxy]carbonyl}amino)-3-(6-oxo-1,6- |
| | dihydropyridin-3-yl)propanoic acid |
| | methyl (2S)-2-({[(9H-fluoren-9-yl)methoxy]carbonyl}amino)-3- |
| | iodopropanoate (CAS: 527696-77-1) |
| | 5-bromo-1,2-dihydropyridin-2-one (CAS: 13466-38-1) |
| | Substantialy identical to Example-3-23-02 |
| 3-35-22 | 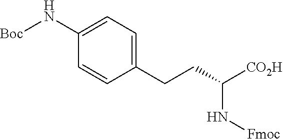 |
| | (2R)-4-(4-{[(tert-butoxy)carbonyl]amino}phenyl)-2-({[(9H-fluoren-9- |
| | yl)methoxy]carbonyl}amino)butanoic acid |
| | methyl (2R)-2-({[(9H-fluoren-9-yl)methoxy]carbonyl}amino)-4- |
| | iodobutanoate (CAS: 1323995-10-3) |
| | tert-butyl N-(4-bromophenyl)carbamat (CAS: 131818-17-2) |
| | Substantialy identical to Example-3-14-03 |
| 3-35-23 | 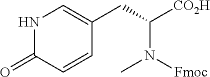 |
| | (2R)-2-({[(9H-fluoren-9-yl)methoxy]carbonyl}(methyl)amino)-3-(6-oxo- |
| | 1,6-dihydropyridin-3-yl)propanoic acid |
| | methyl (2S)-2-({[(9H-fluoren-9-yl)methoxy]carbonyl}amino)-3- |
| | iodopropanoate (CAS: 527696-77-1) |
| | 5-bromo-1,2-dihydropyridin-2-one (CAS: 13466-38-1) |
| | Substantialy identical to Example-3-14-04 |
| 3-35-24 | 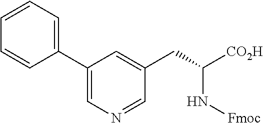 |
| | (2R)-2-({[(9H-fluoren-9-yl)methoxy]carbonyl}amino)-3-(5-phenylpyridin- |
| | 3-yl)propanoic acid |
| | methyl (2S)-2-({[(9H-fluoren-9-yl)methoxy]carbonyl}amino)-3- |
| | iodopropanoate (CAS: 527696-77-1) |
| | 3-bromo-5-phenylpyridine (CAS: 142137-17-5) |
| | Substantialy identical to Example-3-35-16 |
| 3-35-25 | 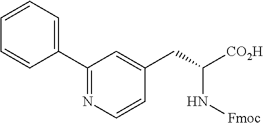 |
| | (2R)-2-({[(9H-fluoren-9-yl)methoxy]carbonyl}amino)-3-(2-phenylpyridin- |
| | 4-yl)propanoic acid |
| | methyl (2S)-2-({[(9H-fluoren-9-yl)methoxy]carbonyl}amino)-3- |
| | iodopropanoate (CAS: 527696-77-1) |
| | 4-bromo-2-phenylpyridine (CAS: 98420-98-5) |
| | Substantialy identical to Example-3-35-07 |
| 3-35-26 | 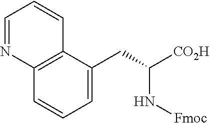 |
| | (2R)-2-({[(9H-fluoren-9-yl)methoxy]carbonyl}amino)-3-(quinolin-5- |
| | yl)propanoic acid |
| | methyl (2S)-2-({[(9H-fluoren-9-yl)methoxy]carbonyl}amino)-3- |
| | iodopropanoate (CAS: 527696-77-1) |
| | 5-bromoquinoline (CAS: 4964-71-0) |
| | Substantialy identical to Example-3-35-08 |
| 3-35-27 | 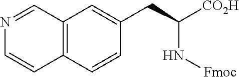 |
| | (2S)-2-({[(9H-fluoren-9-yl)methoxy]carbonyl}amino)-3-(isoquinolin-7- |
| | yl)propanoic acid |
| | Reference example-3-1-01 |
| | 7-bromoisoquinoline (CAS: 58794-09-5) |
| | ¹H NMR (500 MHz, DMSO-d₆) δ 12.95 (br, 1H), 9.79 (s, 1H), 8.68-8.60 |
| | (m, 1H), 8.48-8.32 (m, 2H), 8.31-8.22 (m, 1H), 8.19-8.07 (m, 1H), 7.94- |
| | 7.81 (m, 3H), 7.61-7.50 (m, 2H), 7.49-7.33 (m, 2H), 7.29-7.16 (m, 2H), |
| | 4.45-4.33 (m, 1H), 4.27-4.07 (m, 3H), 3.46-3.32 (m, 1H), 3.25-3.03 (m, |
| | 1H). LC-MS (ESI, m/z): [M + H]⁺ 439. |
| 3-35-28 | 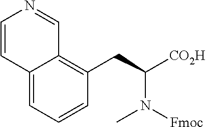 |
| | (2S)-2-({[(9H-fluoren-9-yl)methoxy]carbonyl}(methyl)amino)-3- |
| | (isoquinolin-8-yl)propanoic acid |
| | Reference example-3-1-01 |
| | 8-bromoisoquinoline (CAS: 63927-22-0) |
| | ¹H NMR (400 MHz, DMSO-d₆) δ 13.08 (br, 1H), 9.79-9.58 (m, 1H), 8.61- |
| | 8.45 (m, 1H), 8.08-7.67 (m, 5H), 7.60-7.19 (m, 7H), 5.01-4.89 (m, 1H), |
| | 4.24-4.03 (m, 2H), 3.93-3.31 (m, 3H), 2.76-2.64 (m, 3H). LC-MS (ESI, |
| | m/z): [M + H]⁺ 453. |

### Example-3-36

The following compounds can be synthesized as outlined for the preparation of Example-3-11-05 or Example-3-13-02 employing appropriate starting materials in the table.

| | Structure and IUPAC name |
|---|---|
| | Starting material-A |
| | Starting material-B |
| Example | NMR and MS |
| 3-36-01 | 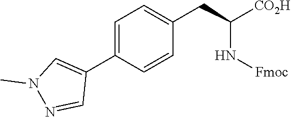 |
| | (2S)-2-({[(9H-fluoren-9-yl)methoxy]carbonyl}amino)-3-[4-(1-methyl-1H- |
| | pyrazol-4-yl)phenyl]propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 4-Bromo-1-methyl-1H-pyrazole (CAS: 15803-02-8) |
| | ¹H NMR (500 MHz, DMSO-d₆) δ 12.75 (br, 1H), 8.12-8.03 (m, 1H), 7.92- |
| | 7.85 (m, 2H), 7.85-7.78 (m, 1H), 7.77-7.34 (m, 7H), 7.34-7.19 (m, 4H), |
| | 4.22-4.11 (m, 4H), 3.85 (s, 3H), 3.09-2.97 (m, 1H), 2.97-2.70 (m, 1H). |
| | LC-MS (ESI, m/z): [M + H]⁺ 468. |
| 3-36-02 | 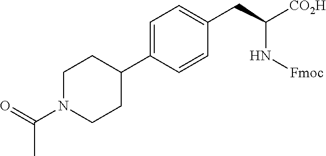 |
| | (2S)-3-[4-(1-acetylpiperidin-4-yl)phenyl]-2-({[(9H-fluoren-9- |
| | yl)methoxy]carbonyl}amino)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | 1-(4-Bromo-3,6-dihydro-1(2H)-pyridinyl)ethanone (CAS: 1622839-80-8) |
| | ¹H NMR (400 MHz, DMSO-d₆) δ 12.74 (br, 1H), 7.88 (d, J = 9.5 Hz, 2H), |
| | 7.78-7.58 (m, 3H), 7.58-7.07 (m, 8H), 4.55-4.42 (m, 1H), 4.28-4.07 (m, |
| | 4H), 3.90-3.77 (m, 1H), 3.15-2.91 (m, 2H), 2.91-2.45 (m, 3H), 2.00 (s, |
| | 3H), 1.80-1.62 (m, 2H), 1.62-1.25 (m, 2H). LC-MS (ESI, m/z): [M + H]⁺ |
| | 513. |
| 3-36-03 | 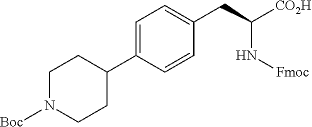 |
| | (2S)-3-(4-{1-[(tert-butoxy)carbonyl]piperidin-4-yl}phenyl)-2-({[(9H- |
| | fluoren-9-yl)methoxy]carbonyl}amino)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | tert-butyl 4-bromo-1,2,3,6-tetrahydropyridine-1-carboxylate (CAS: |
| | 159503-91-0) |
| | ¹H NMR (300 MHz, DMSO-d₆) δ 12.77 (br, 1H), 7.90 (d, J = 7.5 Hz, 2H), |
| | 7.83-7.60 (m, 3H), 7.60-7.01 (m, 8H), 4.35-4.10 (m, 4H), 4.10-3.95 (m, |
| | 2H), 3.22-2.45 (m, 5H), 1.78-1.60 (m, 2H), 1.52-1.31 (m, 11H). LC- |
| | MS (ESI, m/z): [M + H]⁺ 571. |
| 3-36-04 | 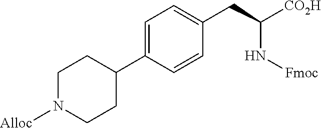 |
| | (2S)-2-({[(9H-fluoren-9-yl)methoxy]carbonyl}amino)-3-(4-{1-[(prop-2- |
| | en-1-yloxy)carbonyl]piperidin-4-yl}phenyl)propanoic acid |
| | (2S)-3-[4-(dihydroxyboranyl)phenyl]-2-({[(9H-fluoren-9- |
| | yl)methoxy]carbonyl}amino)propanoic acid (CAS: 273221-71-9) |
| | prop-2-en-1-yl 4-bromo-1,2,3,6-tetrahydropyridine-1-carboxylate (CAS: |
| | 2300934-47-6) |
| | ¹H NMR (400 MHz, DMSO-d₆) δ 12.75 (br, 1H), 7.88 (d, J = 7.5 Hz, 2H), |
| | 7.82-7.57 (m, 3H), 7.47-7.38 (m, 2H), 7.38-7.22 (m, 2H), 7.22-7.05 (m, |
| | 4H), 6.01-5.87 (m, 1H), 5.36-5.14 (m, 2H), 4.60-4.50 (m, 2H), 4.25- |
| | 4.00 (m, 6H), 3.11-2.96 (m, 1H), 2.96-2.77 (m, 3H), 2.77-2.59 (m, 1H), |
| | 1.78-1.62 (m, 2H), 1.58-1.35 (m, 2H). LC-MS (ESI, m/z): [M + H]⁺ 555. |

### Example-3-37

The following compounds can be synthesized as outlined for the preparation of Example-3-4-07 or Example-3-16-01 or the methods described in Organic Letters (2019), 21(9), 3178. employing appropriate starting materials in the table.

| | Structure and IUPAC name |
|---|---|
| | Starting material-A |
| | Starting material-B |
| Example | NMR and MS |
| 3-37-01 | 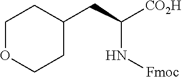 |
| | (2S)-2-({[(9H-fluoren-9-yl)methoxy]carbonyl}amino)-3-(oxan-4- |
| | yl)propanoic acid |
| | 9-chloro-1-[(2S)-1-[(3,4-dichlorophenyl)methyl]pyrrolidine-2-carbonyl]-7-phenyl- |
| | 1H,4H,5H-benzo[d]1-oxa-3,7-diaza-2-nickelacyclononan-4-one |
| | (CAS: 2089661-84-5) |
| | 4-(bromomethyl)oxane (CAS: 125552-89-8) |
| | ¹H NMR (400 MHz, DMSO-d₆) δ 12.69 (br, 1H), 7.91 (d, J = 7.5 Hz, 2H), |
| | 7.79-7.68 (m, 2H), 7.68-7.53 (m, 1H), 7.48-7.39 (m, 2H), 7.38-7.30 (m, |
| | 2H), 4.38-4.20 (m, 3H), 4.03-3.98 (m, 1H), 3.89-3.78 (m, 2H), 3.40- |
| | 3.12 (m, 3H), 1.67-1.52 (m, 4H), 1.27-1.08 (m, 2H). LC-MS (ESI, m/z): |
| | [M − H]⁻ 394. |
| 3-37-02 | 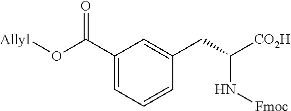 |
| | (2R)-2-({[(9H-fluoren-9-yl)methoxy]carbonyl}amino)-3-{3-[(prop-2-en-1- |
| | yloxy)carbonyl]phenyl}propanoic acid |
| | (2S)-3,6-dimethoxy-2-(propan-2-yl)-2,5-dihydropyrazine |
| | (CAS: 78342-42-4) |
| | prop-2-en-1-yl 3-(chloromethyl)benzoate (CAS: 225122-54-3) |
| | ¹H NMR (500 MHz, DMSO-d₆) δ 12.82 (br, 1H), 8.00-7.77 (m, 5H), 7.77- |
| | 7.36 (m, 6H), 7.36-7.18 (m, 2H), 6.08-5.94 (m, 1H), 5.44-5.37 (m, 1H), |
| | 5.37-5.20 (m, 1H), 4.82-4.72 (m, 2H), 4.28-4.07 (m, 4H), 3.24-3.05 (m, |
| | 1H), 3.05-2.81 (m, 1H). LC-MS (ESI, m/z): [M − H]⁻ 470. |
| 3-37-03 | 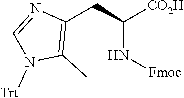 |
| | (2S)-2-({[(9H-fluoren-9-yl)methoxy]carbonyl}amino)-3-[5-methyl-1- |
| | (triphenylmethyl)-1H-imidazol-4-yl]propanoic acid |
| | 9-chloro-1-[(2S)-1-[(3,4-dichlorophenyl)methyl]pyrrolidine-2-carbonyl]-7- |
| | phenyl-1H,4H,5H-benzo[d]1-oxa-3,7-diaza-2-nickelacyclononan-4-one |
| | (CAS: 2089661-84-5) |
| | 4-(Chloromethyl)-5-methyl-1-(triphenylmethyl)-1H-imidazole (CAS: |
| | 106147-85-7) |
| | ¹H NMR (500 MHz, DMSO-d₆) δ 13.7-12.2 (m, 1H), 8.31 (br, 1H), 7.95- |
| | 7.77 (m, 3H), 7.76-7.52 (m, 3H), 7.50-7.36 (m, 10H), 7.36-7.18 (m, 3H), |
| | 7.11-7.00 (m, 5H), 4.40-4.27 (m, 2H), 4.26-4.11 (m, 2H), 3.14-3.03 (m, |
| | 1H), 3.01-2.85 (m, 1H), 1.37 (s, 3H). LC-MS (ESI, m/z): [M + H]⁺ 635. |

### Example-3-38

The following compounds can be synthesized as outlined for the preparation of Example-3-3-01, Example-3-5-04, Example-3-10-07, Example-3-13-04, or Reference example-3-4-01 employing appropriate starting materials in the table.

| | Structure and IUPAC name |
|---|---|
| | Starting material-A |
| | Starting material-B |
| Example | NMR and MS |
| 3-38-01 | 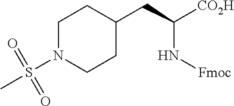 |
| | (2S)-2-({[(9H-fluoren-9-yl)methoxy]carbonyl}amino)-3-(1- |
| | methanesulfonylpiperidin-4-yl)propanoic acid |
| | (2S)-2-({[(9H-fluoren-9-yl)methoxy]carbonyl}amino)-3-(piperidin-4- |
| | yl)propanoic acid (CAS: 368866-16-4) |
| | methanesulfonyl chloride (CAS: 124-63-0) |
| | ¹H NMR (300 MHz, DMSO-d₆) δ 12.63 (br, 1H), 7.90 (d, J = 7.5 Hz, 2H), |
| | 7.79-7.58 (m, 3H), 7.50-7.38 (m, 2H), 7.37-7.26 (m, 2H), 4.39-4.16 (m, |
| | 3H), 4.06-3.96 (m, 1H), 3.60-3.45 (m, 2H), 2.84 (s, 3H), 2.74-2.55 (m, |
| | 2H), 1.84-1.56 (m, 4H), 1.54-1.40 (m, 1H), 1.30-1.05 (m, 2H). LC-MS |
| | (ESI, m/z): [M + Na]⁺ 495. |
| 3-38-02 | 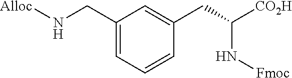 |
| | (2R)-2-({[(9H-fluoren-9-yl)methoxy]carbonyl}amino)-3-[3-({[(prop-2-en- |
| | 1-yloxy)carbonyl]amino}methyl)phenyl]propanoic acid |
| | (2R)-3-[3-(aminomethyl)phenyl]-2-({[(9H-fluoren-9- |
| | yl)methoxy]carbonyl}amino)propanoic acid (CAS: 1270285-96-5) |
| | allyl chloroformate (CAS: 2937-50-0) |
| | ¹H NMR (400 MHz, DMSO-d₆) δ 12.78 (br, 1H), 7.89 (d, J = 7.5 Hz, 2H), |
| | 7.79-7.21 (m, 9H), 7.20-7.07 (m, 3H), 5.99-5.79 (m, 1H), 5.28 (d, J = |
| | 17.2 Hz, 1H), 5.17 (d, J = 10.4 Hz, 1H), 4.49 (d, J = 5.1 Hz, 2H), 4.28-4.09 |
| | (m, 6H), 3.15-2.98 (m, 1H), 2.95-2.74 (m, 1H). LC-MS (ESI, m/z): |
| | [M + H]⁺ 501. |
| 3-38-03 | 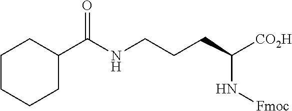 |
| | (2S)-5-(cyclohexylformamido)-2-({[(9H-fluoren-9- |
| | yl)methoxy]carbonyl}amino)pentanoic acid |
| | (2S)-5-amino-2-({[(9H-fluoren-9-yl)methoxy]carbonyl}amino)pentanoic acid |
| | (CAS: 158599-00-9) |
| | Cyclohexanecarboxylic acid (CAS: 98-89-5) |
| | ¹H NMR (500 MHz, DMSO-d₆) δ 12.59 (br, 1H), 7.90 (d, J = 7.90 Hz, 2H), |
| | 7.80-7.13 (m, 8H), 4.37-4.14 (m, 3H), 3.97-3.87 (m, 1H), 3.07-2.93 (m, |
| | 2H), 2.12-2.01 (m, 1H), 1.78-1.05 (m, 14H). LC-MS (ESI, m/z): [M + H]⁺ |
| | 465. |
| 3-38-04 | 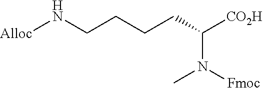 |
| | (2R)-2-({[(9H-fluoren-9-yl)methoxy]carbonyl}(methyl)amino)-6-{[(prop- |
| | 2-en-1-yloxy)carbonyl]amino}hexanoic acid |
| | (2R)-2-({[(9H-fluoren-9-yl)methoxy]carbonyl}amino)-3-(oxan-4- |
| | yl)propanoic acid (CAS: 1793105-27-7) |
| | allyl chloroformate (CAS: 2937-50-0) |
| | ¹H NMR (500 MHz, DMSO-d₆) δ 12.77 (br, 1H), 7.94-7.85 (m, 2H), 7.69- |
| | 7.55 (m, 2H), 7.46-7.37 (m, 2H), 7.37-7.26 (m, 2H), 7.23-7.07 (br, 1H), |
| | 5.97-5.77 (m, 1H), 5.30-5.11 (m, 2H), 4.53-4.21 (m, 6H), 3.03-2.90 (m, |
| | 2H), 2.77-2.66 (m, 3H), 1.86-1.52 (m, 2H), 1.50-1.28 (m, 2H), 1.25- |
| | 0.98 (m, 2H). LC-MS (ESI, m/z): [M + H]⁺ 467. |

### Example-3-39

The following compounds can be synthesized as outlined for the preparation of Example-3-12-02, Example-3-12-04, or Reference example-3-5-01 employing appropriate starting materials in the table.

| | Structure and IUPAC name |
|---|---|
| | Starting material-A |
| | Starting material-B |
| Example | NMR and MS |
| 3-39-01 | 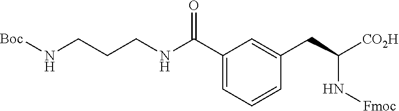 |
| | (2S)-3-{3-[(3-{[(tert-butoxy)carbonyl]amino}propyl)carbamoyl]phenyl}- |
| | 2-({[(9H-fluoren-9-yl)methoxy]carbonyl}amino)propanoic acid |
| | (2S)-3-{3-[(tert-butoxy)carbonyl]phenyl}-2-({[(9H-fluoren-9- |
| | yl)methoxy]carbonyl}amino)propanoic acid (CAS: 210282-33-0) |
| | tert-Butyl (3-aminopropyl)carbamate (CAS: 75178-96-0) |
| | ¹H NMR (400 MHz, DMSO-d₆) δ 13.05 (br, 1H), 8.51-8.29 (m, 1H), 7.92- |
| | 7.77 (m, 3H), 7.75-7.47 (m, 4H), 7.46-7.15 (m, 6H), 6.88-6.35 (m, 1H), |
| | 4.34-3.99 (m, 4H), 3.35-3.08 (m, 3H), 3.04-2.90 (m, 3H), 1.73-1.55 (m, |
| | 2H), 1.37 (s, 9H). LC-MS (ESI, m/z): [M + H]⁺ 588. |
| 3-39-02 | 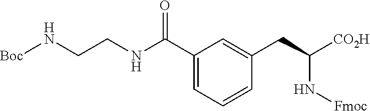 |
| | (2S)-3-{3-[(2-{[(tert-butoxy)carbonyl]amino}ethyl)carbamoyl]phenyl}-2- |
| | ({[(9H-fluoren-9-yl)methoxy]carbonyl}amino)propanoic acid |
| | (2S)-3-{3-[(tert-butoxy)carbonyl]phenyl}-2-({[(9H-fluoren-9- |
| | yl)methoxy]carbonyl}amino)propanoic acid (CAS: 210282-33-0) |
| | tert-butyl N-(2-aminoethyl)carbamate (CAS: 57260-73-8) |
| | ¹H NMR (400 MHz, DMSO-d₆) δ 13.05 (br, 1H), 8.51-8.29 (m, 1H), 7.92- |
| | 7.47 (m, 7H), 7.46-7.15 (m, 6H), 6.96-6.85 (m, 1H), 4.24-4.08 (m, 4H), |
| | 3.35-3.25 (m, 2H), 3.25-3.03 (m, 3H), 3.03-2.84 (m, 1H), 1.37 (s, 9H). |
| | LC-MS (ESI, m/z): [M − Boc + H]⁺ 474. |
| 3-39-03 | 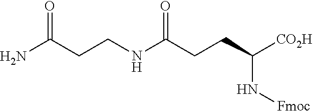 |
| | (2S)-4-[(2-carbamoylethyl)carbamoyl]-2-({[(9H-fluoren-9- |
| | yl)methoxy]carbonyl}amino)butanoic acid |
| | (2S)-5-(tert-butoxy)-2-({[(9H-fluoren-9-yl)methoxy]carbonyl}amino)-5- |
| | oxopentanoic acid (CAS: 71989-18-9) |
| | 3-aminopropanamide (CAS: 4726-85-6) |
| | ¹H NMR (300 MHz, DMSO-d₆) δ 12.66 (br, 1H), 7.95-7.81 (m, 3H), 7.81- |
| | 7.57 (m, 3H), 7.46-7.28 (m, 5H), 6.80 (br, 1H), 4.34-4.17 (m, 3H), 3.88- |
| | 3.76 (m, 1H), 3.31-2.97 (m, 2H), 2.42-2.28 (m, 4H), 2.06-1.88 (m, 1H), |
| | 1.88-1.67 (m, 1H). LC-MS (ESI, m/z): [M + H]⁺ 440. |
| 3-39-04 | 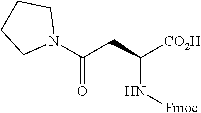 |
| | (2S)-2-({[(9H-fluoren-9-yl)methoxy]carbonyl}amino)-4-oxo-4-(pyrrolidin- |
| | 1-yl)butanoic acid |
| | (S)-3-((((9H-fluoren-9-yl)methoxy)carbonyl)amino)-4-(allyloxy)-4- |
| | oxobutanoic acid (CAS: 144120-53-6) |
| | Pyrrolidine (CAS: 123-75-1) |
| | ¹H NMR (500 MHz, DMSO-d₆) δ 12.64 (br, 1H), 7.89 (d, J = 7.5 Hz, 2H), |
| | 7.71 (d, J = 7.5 Hz, 2H), 7.47-7.38 (m, 3H), 7.38-7.27 (m, 2H), 4.47-4.38 |
| | (m, 1H), 4.32-4.18 (m, 3H), 3.43-3.35 (m, 2H), 3.31-3.22 (m, 2H), 2.71- |
| | 2.61 (m, 2H), 1.91-1.81 (m, 2H), 1.81-1.69 (m, 2H). LC-MS (ESI, m/z): |
| | [M + Na]⁺ 409. |
| 3-39-05 | 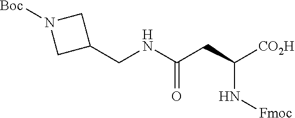 |
| | (2S)-3-[({1-[(tert-butoxy)carbonyl]azetidin-3-yl}methyl)carbamoyl]-2- |
| | ({[(9H-fluoren-9-yl)methoxy]carbonyl}amino)propanoic acid |
| | (S)-3-((((9H-fluoren-9-yl)methoxy)carbonyl)amino)-4-(allyloxy)-4- |
| | oxobutanoic acid (CAS: 144120-53-6) |
| | tert-butyl 3-(aminomethyl)azetidine-1-carboxylate (CAS: 325775-44-8) |
| | ¹H NMR (500 MHz, DMSO-d₆) δ 12.66 (br, 1H), 8.08-7.96 (m, 1H), 7.89 |
| | (d, J = 7.6 Hz, 2H), 7.76-7.66 (m, 2H), 7.58-7.52 (m, 1H), 7.46-7.37 (m, |
| | 2H), 7.37-7.29 (m, 2H), 4.40-4.32 (m, 1H), 4.32-4.26 (m, 2H), 4.25- |
| | 4.13 (m, 1H), 3.80 (br, 2H), 3.48 (br, 2H), 3.28-3.12 (m, 2H), 2.69-2.53 |
| | (m, 2H), 2.48-2.38 (m, 1H), 1.35 (s, 9H). LC-MS (ESI, m/z): [M + H]⁺ 524. |

### Example-3-40

The following compounds can be synthesized as outlined for the preparation of Example-3-1-02. Example-3-1-04, or Example-3-1-08 employing appropriate starting materials in the table.

| | Structure and IUPAC name |
|---|---|
| | Starting material-A |
| | Starting material-B |
| Example | NMR and MS |
| 3-40-01 | 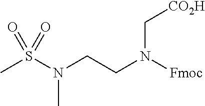 |
| | 2-({[(9H-fluoren-9-yl)methoxy]carbonyl}[2-(N- |
| | methylmethanesulfonamido)ethyl]amino)acetic acid |
| | tert-butyl bromoacetate (CAS: 5292-43-3) |
| | N-(2-aminoethyl)-N-methylmethanesulfonamide (CAS: 778572-84-2) |
| | ¹H NMR (500 MHz, DMSO-d₆) δ 12.78 (br, 1H), 7.94-7.86 (m, 2H), 7.72- |
| | 7.59 (m, 2H), 7.46-7.39 (m, 2H), 7.38-7.28 (m, 2H), 4.48 (d, J = 5.7 Hz, |
| | 1H), 4.34-4.19 (m, 2H), 4.01 (s, 1H), 3.87 (m, 1H), 3.51-3.42 (m, 1H), |
| | 3.25-3.14 (m, 2H), 2.97-2.90 (m, 1H), 2.89-2.46 (m, 6H). LC-MS (ESI, |
| | m/z): [M + H]⁺ 433. |
| 3-40-02 | 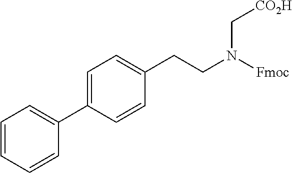 |
| | 2-[(2-{[1,1′-bipheny]-4-yl}ethyl)({[(9H-fluoren-9- |
| | yl)methoxy]carbonyl})amino]acetic acid |
| | tert-butyl bromoacetate (CAS: 5292-43-3) |
| | [1,1′-Biphenyl]-4-ethanamine (CAS: 17027-51-9) |
| | ¹H NMR (300 MHz, DMSO-d₆) δ 12.43 (br, 1H), 7.90 (t, J = 7.4 Hz, 2H), |
| | 7.76-7.22 (m, 14H), 7.01 (d, J = 7.9 Hz, 1H), 4.51 (d, J = 5.3 Hz, 1H), 4.41- |
| | 4.13 (m, 2H), 4.02-3.75(m, 2H), 3.49 (t, J = 7.6 Hz, 1H), 3.20-3.09 (m, |
| | 1H), 2.83 (t, J = 7.7 Hz, 1H), 2.61-2.38 (m, 1H). LC-MS (ESI, m/z): [M + H]⁺ |
| | 478. |

### Example-3-41

The following compounds can be synthesized as outlined for the preparation of Example-3-21-01, Example-3-21-02, or Example-3-31-01 employing appropriate starting materials in the table.

| | Structure and IUPAC name |
|---|---|
| | Starting material-A |
| | Starting material-B |
| Example | NMR and MS |
| 3-41-01 | 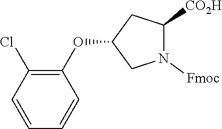 |
| | (2S,4R)-1-(((9H-fluoren-9-yl)methoxy)carbonyl)-4-(2- |
| | chlorophenoxy)pyrrolidine-2-carboxylic acid |
| | 1-(tert-butyl) 2-methyl (2S,4S)-4-hydroxypyrrolidine-1,2-dicarboxylate |
| | (CAS: 102195-79-9) |
| | 2-chlorophenol (CAS: 95-57-8) |
| | ¹H NMR (500 MHz, DMSO-d₆) δ 12.89 (br, 1H), 7.93-7.80 (m, 2H), 7.70- |
| | 7.63 (m, 1H), 7.58-7.52 (m, 1H), 7.49-7.29 (m, 5H), 7.28-7.18 (m, 2H), |
| | 7.07-7.00 (m, 1H), 5.16 (br, 1H), 4.57-4.14 (m, 4H), 3.75-3.64 (m, 2H), |
| | 2.65-2.51 (m, 1H), 2.37-2.18 (m, 1H). LC-MS (ESI, m/z): [M + H]⁺ 464. |
| 3-41-02 | 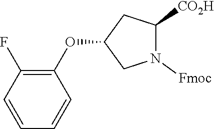 |
| | (2S,4R)-1-(((9H-fluoren-9-yl)methoxy)carbonyl)-4-(2- |
| | fluorophenoxy)pyrrolidine-2-carboxylic acid |
| | 1-(tert-butyl) 2-methyl (2S,4S)-4-hydroxypyrrolidine-1,2-dicarboxylate |
| | (CAS: 102195-79-9) |
| | 2-fluorophenol (CAS: 367-12-4) |
| | ¹H NMR (500 MHz, DMSO-d₆) δ 12.89 (br, 1H), 7.91-7.82 (m, 2H), 7.71- |
| | 7.62 (m, 1H), 7.60-7.54 (m, 1H), 7.44-7.13 (m, 7H), 7.07-6.99 (m, 1H), |
| | 5.09 (br, 1H), 4.57-4.17 (m, 4H), 3.80-3.66 (m, 2H), 2.65-2.52 (m, 1H), |
| | 2.38-2.17 (m, 1H). LC-MS (ESI, m/z): [M + H]⁺ 448. |
| 3-41-03 | 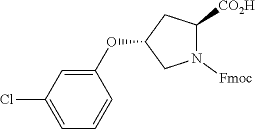 |
| | (2S,4R)-1-(((9H-fluoren-9-yl)methoxy)carbonyl)-4-(3- |
| | chlorophenoxy)pyrrolidine-2-carboxylic acid |
| | 1-(tert-butyl) 2-methyl (2S,4S)-4-hydroxypyrrolidine-1,2-dicarboxylate |
| | (CAS: 102195-79-9) |
| | 3-chlorophenol (CAS: 108-43-0) |
| | ¹H NMR (500 MHz, DMSO-d₆) δ 12.89 (br, 1H), 7.92-7.79 (m, 2H), 7.70- |
| | 7.63 (m, 1H), 7.57-7.51 (m, 1H), 7.44-7.16 (m, 5H), 7.10-7.03 (m, 2H), |
| | 6.97-6.92 (m, 1H), 5.10 (br, 1H), 4.49-4.32 (m, 1H), 4.29-4.16 (m, 3H), |
| | 3.74-3.59 (m, 2H), 2.64-2.44 (m, 1H), 2.38-2.16 (m, 1H). LC-MS (ESI, |
| | m/z): [M + H]⁺ 464. |
| 3-41-04 | 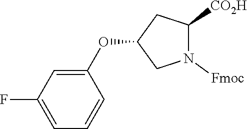 |
| | (2S,4R)-1-(((9H-fluoren-9-yl)methoxy)carbonyl)-4-(3- |
| | fluorophenoxy)pyrrolidine-2-carboxylic acid |
| | 1-(tert-butyl) 2-methyl (2S,4S)-4-hydroxypyrrolidine-1,2-dicarboxylate |
| | (CAS: 102195-79-9) |
| | 3-fluorophenol (CAS: 372-20-3) |
| | ¹H NMR (500 MHz, DMSO-d₆) δ 12.80 (m, 1H), 7.92-7.80 (m, 2H), 7.70- |
| | 7.63 (m, 1H), 7.59-7.52 (m, 1H), 7.44-7.31 (m, 4H), 7.27-7.18 (m, 1H), |
| | 6.89-6.79 (m, 3H), 5.09 (br, 1H), 4.49-4.17 (m, 4H), 3.75-3.63 (m, 2H), |
| | 2.63-2.44 (m, 1H), 2.37-2.17 (m, 1H). LC-MS (ESI, m/z): [M + H]⁺ 448. |
| 3-41-05 | 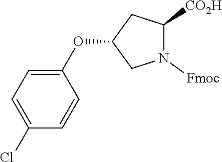 |
| | (2S,4R)-1-(((9H-fluoren-9-yl)methoxy)carbonyl)-4-(4- |
| | chlorophenoxy)pyrrolidine-2-carboxylic acid |
| | 1-(tert-butyl) 2-methyl (2S,4S)-4-hydroxypyrrolidine-1,2-dicarboxylate |
| | (CAS: 102195-79-9) |
| | 4-chlorophenol (CAS: 106-48-9) |
| | ¹H NMR (500 MHz, DMSO-d₆) δ 13.12-12.69 (m, 1H), 7.91-7.83 (m, |
| | 2H), 7.69-7.63 (m, 1H), 7.57-7.52 (m, 1H), 7.44-7.31 (m, 5H), 7.28- |
| | 7.18 (m, 1H), 7.02-6.97 (m, 2H), 5.06 (br, 1H), 4.49-4.16 (m, 4H), 3.73- |
| | 3.61 (m, 2H), 2.65-2.44 (m, 1H), 2.48-2.17 (m, 1H). LC-MS (ESI, m/z): |
| | [M + H]⁺ 464. |
| 3-41-06 | 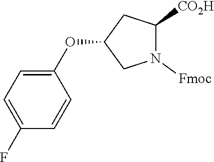 |
| | (2S,4R)-1-(((9H-fluoren-9-yl)methoxy)carbonyl)-4-(4- |
| | fluorophenoxy)pyrrolidine-2-carboxylic acid |
| | 1-(tert-butyl) 2-methyl (2S,4S)-4-hydroxypyrrolidine-1,2-dicarboxylate |
| | (CAS: 102195-79-9) |
| | 4-fluorophenol (CAS: 371-41-5) |
| | ¹H NMR (500 MHz, DMSO-d₆) δ 12.88 (br, 1H), 7.92-7.83 (m, 2H), 7.71- |
| | 7.61 (m, 1H), 7.59-7.53 (m, 1H), 7.44-7.30 (m, 3H), 7.28-7.12 (m, 3H), |
| | 7.02-6.96 (m, 2H), 5.02 (br, 1H), 4.50-4.12 (m, 4H), 3.73-3.61 (m, 2H), |
| | 2.60-2.43 (m, 1H), 2.35-2.15 (m, 1H). LC-MS (ESI, m/z): [M + H]⁺ 448. |

### Example-3-42: (2S)-3-[1-(3-{[(tert-butoxy)carbonyl]amino}propyl)-1H-indol-3-yl]-2-({[(9H-fluoren-9-yl)methoxy]carbonyl}amino)propanoic acid

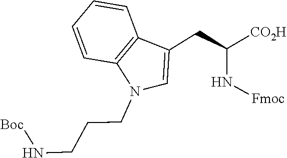

To a solution of (2S)-2-[[(benzyloxy)carbonyl]amino]-3-(1H-indol-3-yl)propanoic acid (CAS: 7432-21-5, 20.0 g, 59.1 mmol) in DMF (100 mL) and THF (100 mL) at 0° C. were added potassium tert-butoxide (13.3 g, 118 mmol). The mixture was stirred at rt for 30 min. To the mixture was added tert-butyl (3-bromopropyl)carbamate (CAS: 83948-53-2, 14.8 g, 62.1 mmol) at 0° C. The mixture was stirred at rt for 3 h. The reaction mixture at 0° C. was quenched with H₂O. The aqueous phase was extracted three times with EtOAc. The combined organic extracts were washed three times with brine, dried over Na₂SO₄, filtered, and then concentrated. The resulting residue was purified by silica gel flash column chromatography (isocratic, petroleum ether/EtOAc=25/75). Appropriate fractions were concentrated. To a solution of the obtained material (10.0 g, 20.2 mmol) in MeOH (100 mL) at rt was added Pd/C (4.5 g, wet). The mixture was stirred at rt for 2 h under H₂ atmosphere, and then filtered. The filtrate was concentrated. To a solution of the obtained material (10.0 g, 27.7 mmol) in 1,4-dioxane (90.0 mL) and H₂O (16.0 mL) at 0° C. was added NaHCO₃ (4.65 g, 55.3 mmol), followed by Fmoc-OSu (9.33 g, 27.7 mmol). The mixture was stirred at rt for 3 h. The pH of the mixture was adjusted to pH 5-6 with 20% aq. citric acid. The mixture was extracted three times with EtOAc. The organic extracts were washed with brine, dried over Na₂SO₄, filtered, and then concentrated. The resulting residue was purified by silica gel flash column chromatography (isocratic, petroleum ether/EtOAc=20/80) to afford the title compound (10.6 g, 18.1 mmol). ¹H NMR (400 MHz, DMSO-d₆) δ 12.71 (br, 1H), 7.88 (d, J=7.2 Hz, 2H), 7.76-7.62 (m, 3H), 7.57 (d, J=7.8 Hz, 1H), 7.52-7.35 (m, 3H), 7.35-7.22 (m, 2H), 7.21 (s, 1H), 7.16-6.90 (m, 3H), 4.37-4.01 (m, 6H), 3.24-2.96 (m, 2H), 2.95-2.80 (m, 2H), 1.87-1.71 (m, 2H), 1.36 (s, 9H). LC-MS (ESI, m/z): [M−Boc+H]⁺ 484.

### Example-3-43-01: (2R)-3-{[(2,4-dimethoxyphenyl)methyl]sulfamoyl}-2-({[(9H-fluoren-9-yl)methoxy]carbonyl}amino)propanoic acid

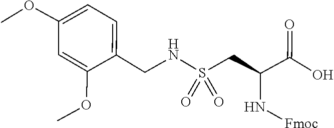

To a solution of (2R)-2-({[(9H-fluoren-9-yl)methoxy]carbonyl}amino)-3-[(triphenylmethyl)sulfanyl]propanoic acid (CAS: 103213-32-7, 20.0 g, 34.1 mmol) in DMF (200 mL) at 0° C. were added potassium carbonate (14.2 g, 102 mmol) and a solution of iodomethane (14.5 g, 102 mmol) in DMF (50.0 mL). The mixture was stirred at rt for 3 h. The reaction was then quenched with H₂O. The mixture was extracted with EtOAc. The organic extracts were washed with brine, dried over Na₂SO₄, filtered, and concentrated. To a solution of the obtained material (7.0 g, 11.7 mmol) in MeCN (70 mL) at rt was added H₂O (0.63 g, 35.0 mmol). To the mixture at 0° C. was added tert-butyl hypochlorite (CAS: 507-40-4, 3.80 g, 35.0 mmol) dropwise. The mixture was stirred at 0° C. for 15 min. To the mixture at 0° C. was added 1-(2,4-dimethoxyphenyl) methanamine (CAS: 20781-20-8, 7.81 g, 46.7 mmol). The mixture was stirred at rt for 1 h. The mixture was concentrated. The resulting residue was purified by silica gel flash column chromatography (isocratic, petroleum ether/EtOAc=67/33). Appropriate fractions were collected and concentrated. To a solution of the obtained material (4.40 g, 7.93 mmol) in ClCH₂CH₂Cl (40.0 mL) was added trimethyltin hydroxide (2.87 g, 15.9 mmol). The resulting mixture was stirred at 60° C. for 16 h. The mixture was concentrated. The resulting residue was dissolved in EtOAc. The mixture was washed with 1M aq. HCl, brine, dried over Na₂SO₄, filtered, and then concentrated. The resulting residue was purified by silica gel flash column chromatography (isocratic, CH₂Cl₂/MeOH=97/3) to afford the title compound (2.33 g, 4.22 mmol). ¹H NMR (400 MHz, DMSO-d₆) δ 13.11 (br, 1H), 7.95-7.78 (m, 3H), 7.72 (d, J=7.5 Hz, 2H), 7.58-7.26 (m, 5H), 7.19 (d, J=8.3 Hz, 1H), 6.57-6.44 (m, 2H), 4.45-4.37 (m, 1H), 4.37-4.15 (m, 3H), 4.11-4.03 (m, 2H), 3.83-3.73 (m, 6H), 3.52-3.15 (m, 2H). LC-MS (ESI, m/z): [M−H]⁻ 539.

### Example-3-43

The following compounds can be synthesized as outlined for the preparation of Example-3-43-01 employing appropriate starting materials in the table.

| | Structure and IUPAC name |
|---|---|
| | Starting material-A |
| Ex- | Starting material-B |
| ample | NMR and MS |
| 3-43-02 | 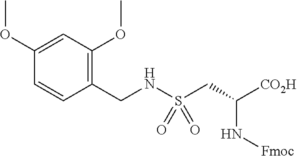 |
| | (2S)-3-{[(2,4-dimethoxyphenyl)methyl]sulfamoyl}-2-({[(9H- |
| | fluoren-9-yl)methoxy]carbonyl}amino)propanoic acid |
| | (2S)-2-({[(9H-fluoren-9-yl)methoxy]carbonyl}amino)-3- |
| | [(triphenylmethyl)sulfanyl]propanoic acid (CAS: 167015-11-4) |
| | 2,4-Dimethoxybenzylamine (CAS: 20781-20-8) |
| | Substantialy identical to Example-3-43-01 |

### Example-3-44: (2S)-2-({[(9H-fluoren-9-yl)methoxy]carbonyl}(methyl)amino)-4-propoxybutanoic acid

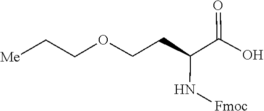

The solution of Reference example-3-2-03 (18.0 g, 39.2 mmol) and sodium dihydrogen phosphate (CAS: 7558-80-7, 18.8 g, 157 mmol) in 1-propanol (CAS: 71-23-8, 150 mL) was stirred at 40-45° C. for 5.5 h. The resulting mixture was concentrated. The residue was dissolved with EtOAc and water. The resulting mixture was extracted with EtOAc. The combined organic extracts were washed with brine, dried over Na₂SO₄, filtered, and then concentrated. To the solution of the obtained material (17.2 g, 39.2 mmol) in DCM (73.5 mL) at 0° C. was added TFA (24.5 mL). The mixture was stirred at rt for 1.5 h, and then concentrated. The residue was azetroped with toluene and then dissolved with EtOAc. The solution was washed with 0.5M aq. NaH₂PO₄ and brine. The pH of water layer was adjusted to 4 with satd. aq. NaHCO₃. The resulting mixture was extracted with EtOAc. The combined organic extracts were washed with brine, dried over Na₂SO₄, filtered, and then concentrated. The residue was purified by flash column chromatography (gradient, CH₂Cl₂/MeOH=98/2 to 96/4) to afford the title compound (13.1 g, 34.2 mmol). ¹H NMR (500 MHz, DMSO-d₆) δ 12.61 (br, 1H), 7.90 (d, J=7.6 Hz, 2H), 7.77-7.68 (m, 2H), 7.66-7.55 (m, 1H), 7.47-7.38 (m, 2H), 7.36-7.27 (m, 2H), 4.33-4.14 (m, 3H), 4.11-4.02 (m, 1H), 3.45-3.36 (m, 2H), 3.36-3.19 (m, 2H), 2.01-1.92 (m, 1H), 1.82-1.71 (m, 1H), 1.54-1.42 (m, 2H), 0.89-0.79 (m, 3H). LC-MS (ESI, m/z): [M+H]⁺ 384.

### Example-3-45: (2S)-2-({[(9H-fluoren-9-yl)methoxy]carbonyl}amino)-3-[4-({[(prop-2-en-1-yloxy)carbonyl]amino}methyl)phenyl]propanoic acid

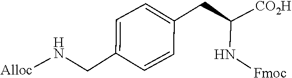

To a mixture of (2S)-3-[4-({[(tert-butoxy)carbonyl]amino}methyl)phenyl]-2-({[(9H-fluoren-9-yl)methoxy]carbonyl}amino)propanoic acid (CAS: 204715-91-3, 8.00 g, 15.49 mmol) in DCM (50.00 mL) at rt was added 4M HCl in 1,4-dioxane (50.00 mL). The resulting solution was stirred at rt for 1 h. The resulting mixture was concentrated. To a mixture of the obtained material (6.47 g, 14.3 mmol) and Na₂CO₃ (4.11 g, 38.4 mmol) in THF (50 mL) and H2O (50 mL) at 0° C. was added allyl chlorocarbonate (2.80 g, 23.2 mmol) dropwise with stirring. The resulting solution was stirred at rt for 3 h. The reaction was then quenched with water. The resulting solution was extracted with EtOAc. The combined organic extracts were washed with brine, dried over Na₂SO₄, filtered, and concentrated. The residue was purified by reversed phase silica gel flash column chromatography (C18 silica gel, gradient, H₂O/CH₃CN with 1% formic acid=50/50 to 20/80) to afford the title compound (5.21 g, 10.4 mmol). ¹H NMR (400 MHz, DMSO-d₆): δ 12.79 (s, 1H), 7.97-7.82 (m, 2H), 7.79-7.47 (m, 4H), 7.46-7.26 (m, 4H), 7.25-7.12 (m, 4H), 5.99-5.78 (m, 1H), 5.35-5.05 (m, 2H), 4.56-4.45 (m, 2H), 4.29-4.08 (m, 6H), 3.07 (dd, J=13.8, 4.4 Hz, 1H), 2.87 (dd, J=13.8, 10.4 Hz, 1H). LC-MS (ESI, m/z): [M+H]⁺ 501.

### [Polypeptide]

The polypeptide of this disclosure comprises at least one amino acid described herein above.

### [Derivatives or Analogues]

It is possible for skilled person to synthesis the derivatives, analogues or similar compound in the similar manner. These compounds are also included as a part of this invention.

What is claimed is:

1. An amino acid derivative selected from the group consisting of

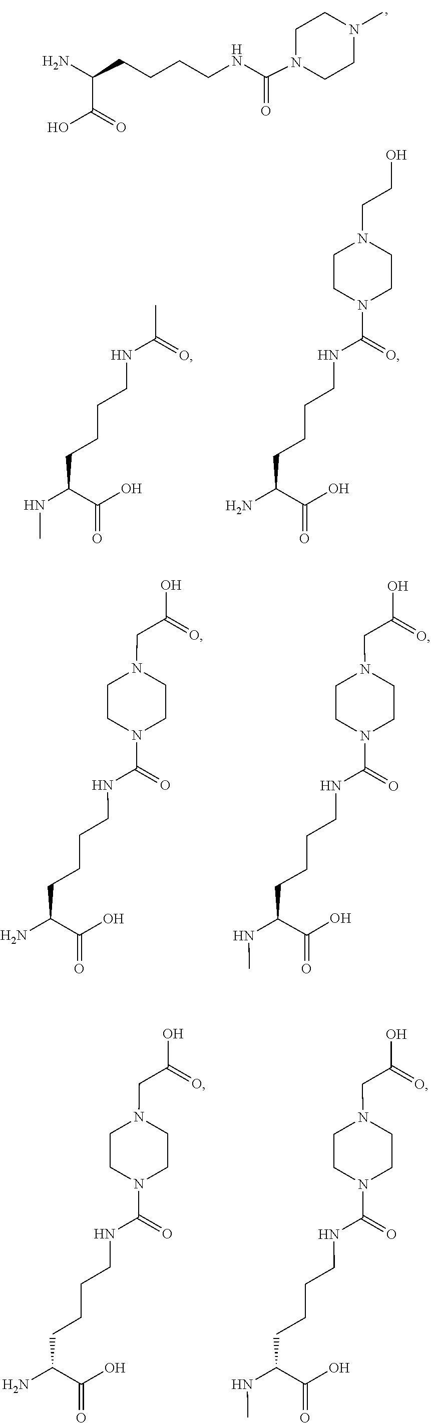

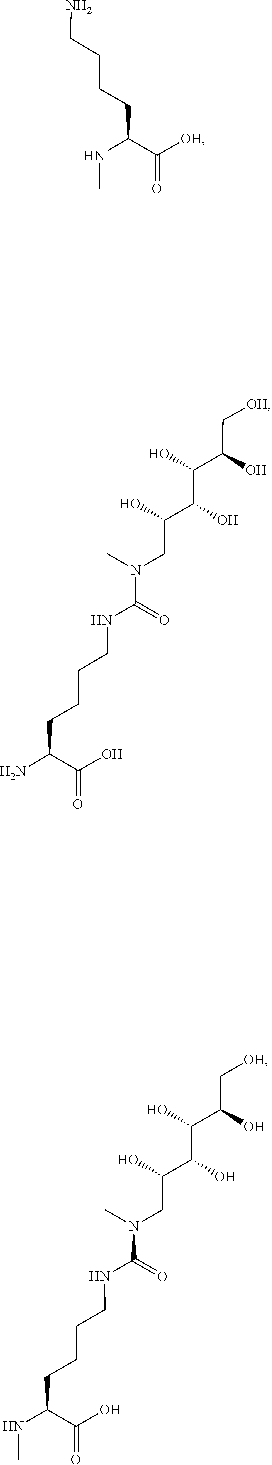

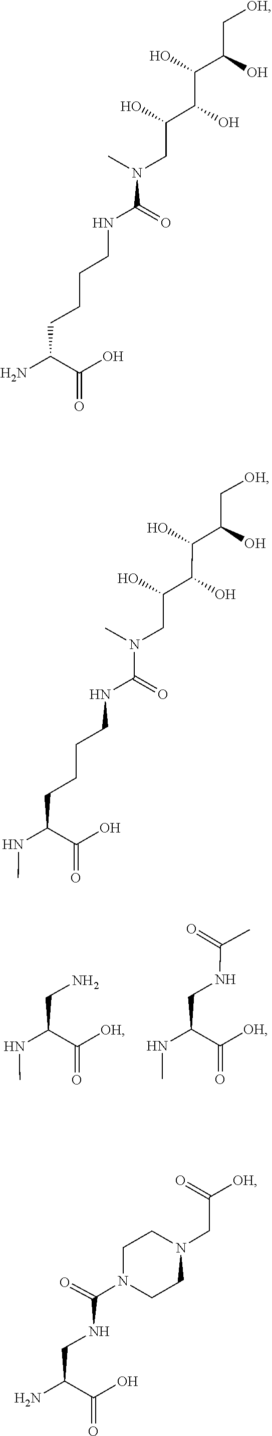

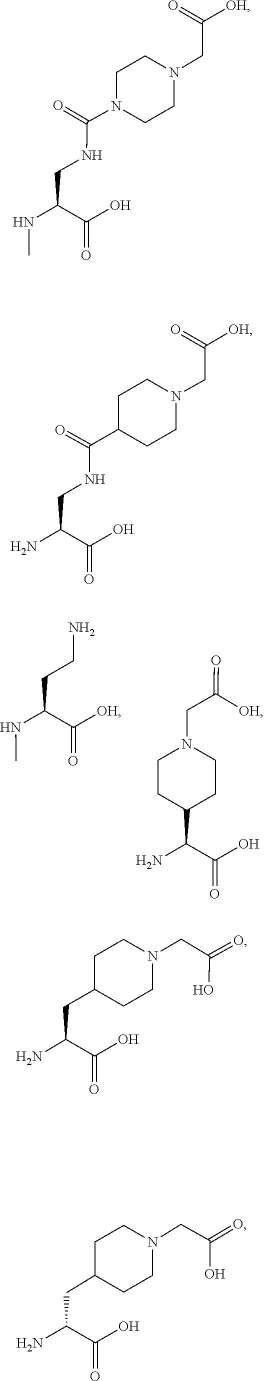

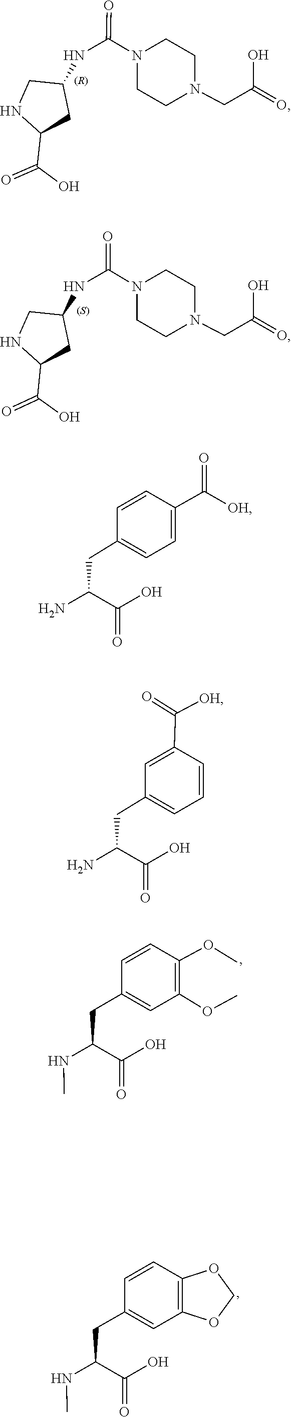

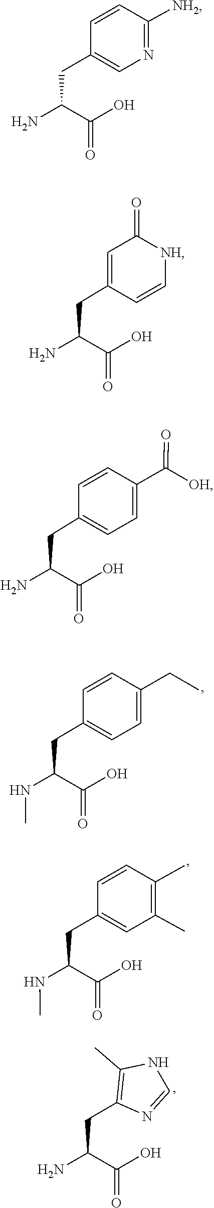

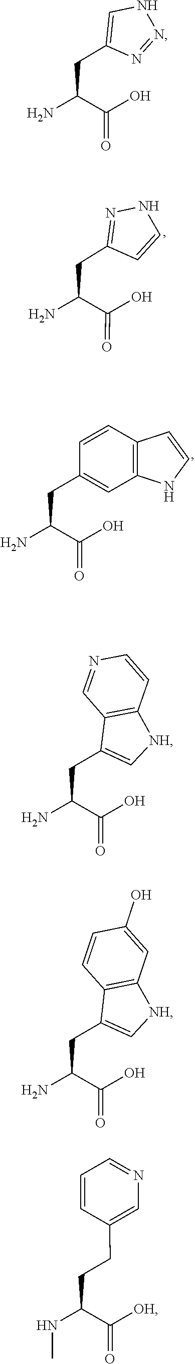

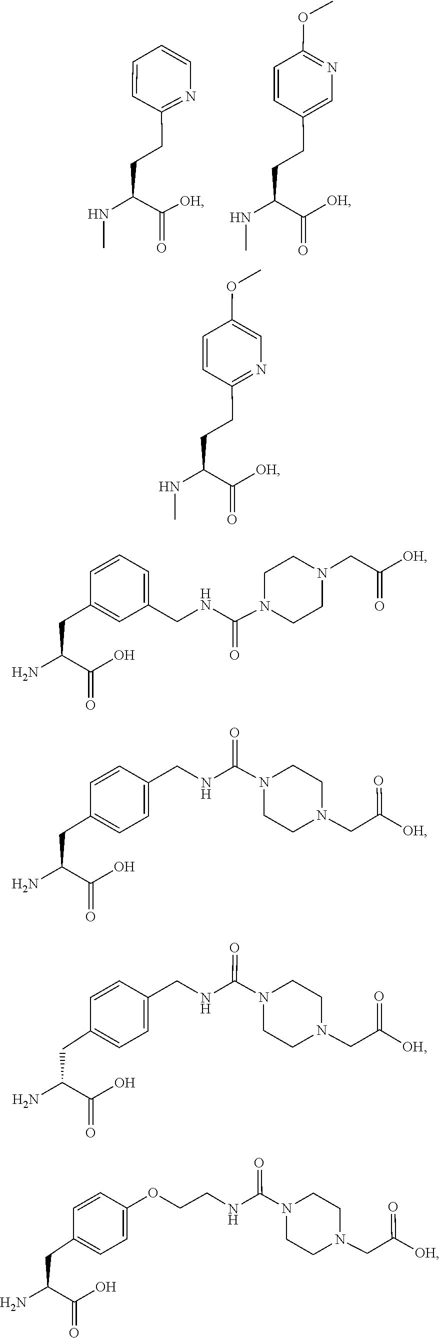

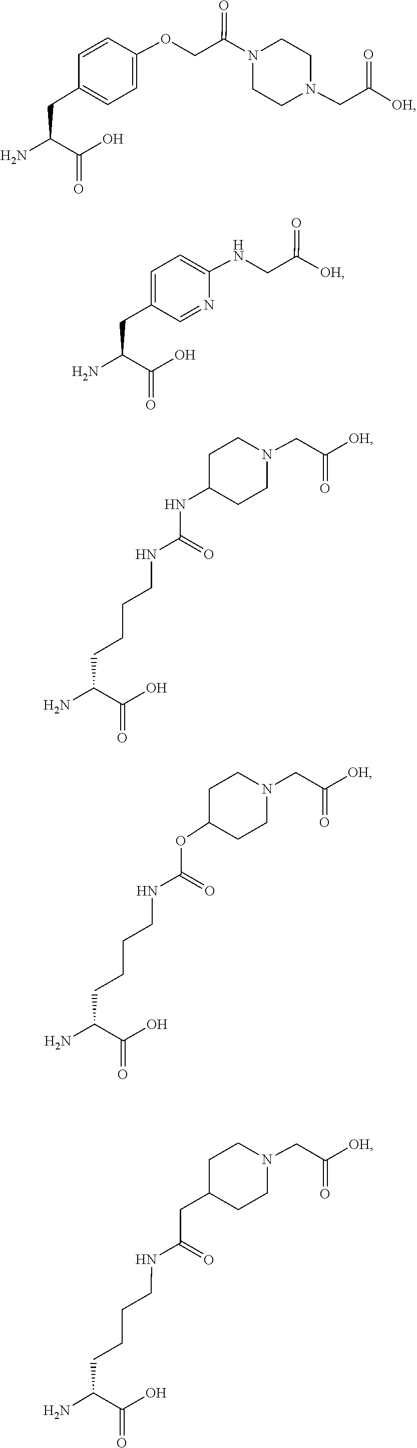 [SMILES: N[C@@H](Cc1ccc(OCC(=O)N2CCN(CC(=O)O)CC2)cc1)C(=O)O ; N[C@@H](Cc1ccc(NCC(=O)O)nc1)C(=O)O ; N[C@H](CCCCNC(=O)NC1CCN(CC(=O)O)CC1)C(=O)O ; N[C@H](CCCCNC(=O)OC1CCN(CC(=O)O)CC1)C(=O)O ; N[C@H](CCCCNC(=O)CC1CCN(CC(=O)O)CC1)C(=O)O]

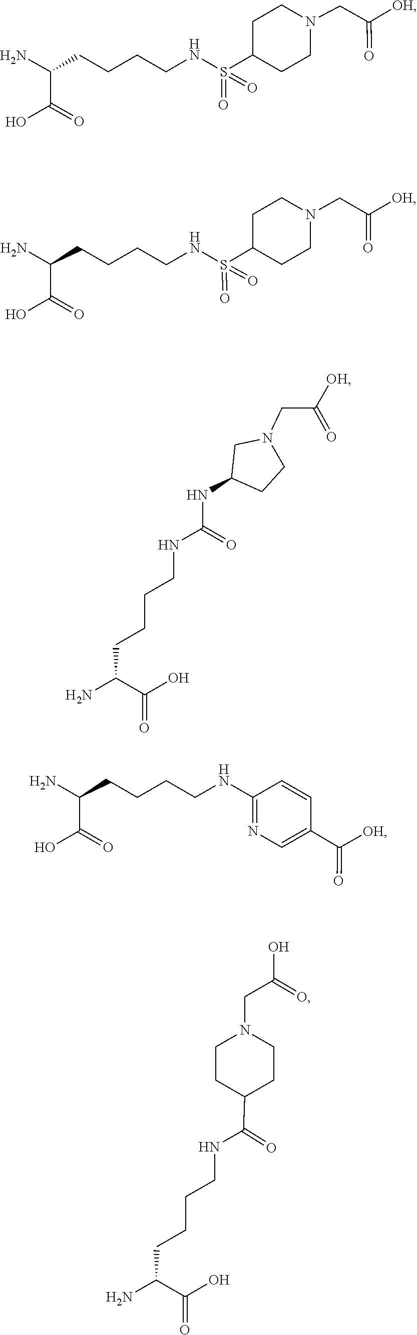 [SMILES: N[C@H](CCCCNS(=O)(=O)C1CCN(CC(=O)O)CC1)C(=O)O ; N[C@@H](CCCCNS(=O)(=O)C1CCN(CC(=O)O)CC1)C(=O)O ; N[C@H](CCCCNC(=O)N[C@@H]1CCN(CC(=O)O)C1)C(=O)O ; N[C@@H](CCCCNc1ccc(C(=O)O)cn1)C(=O)O ; N[C@H](CCCCNC(=O)C1CCN(CC(=O)O)CC1)C(=O)O]

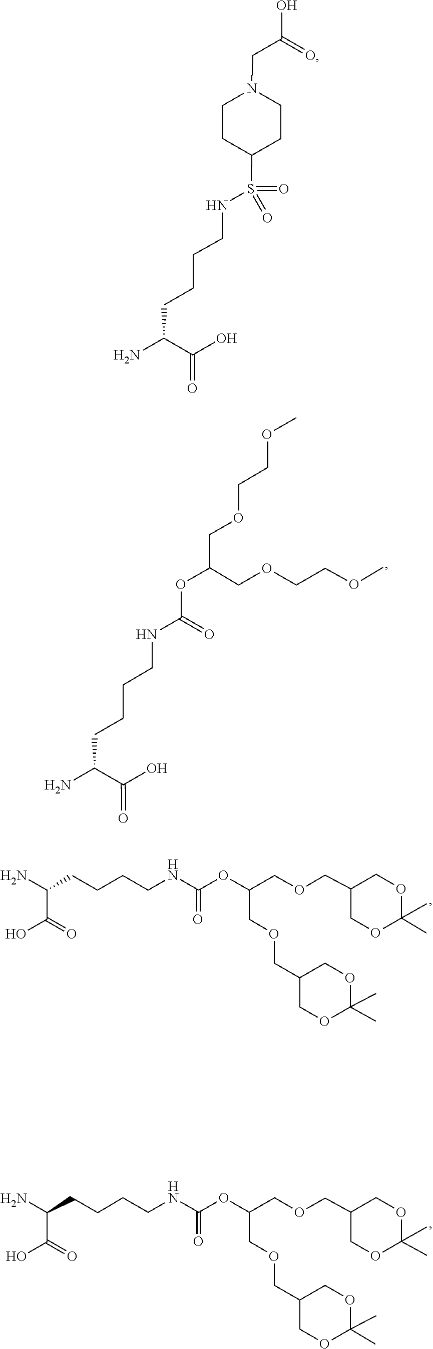 [SMILES: N[C@H](CCCCNS(=O)(=O)C1CCN(CC(=O)O)CC1)C(=O)O ; COCCOCC(COCCOC)OC(=O)NCCCC[C@@H](N)C(=O)O ; CC1(C)OCC(COCC(COCC2COC(C)(C)OC2)OC(=O)NCCCC[C@@H](N)C(=O)O)CO1 ; CC1(C)OCC(COCC(COCC2COC(C)(C)OC2)OC(=O)NCCCC[C@H](N)C(=O)O)CO1]

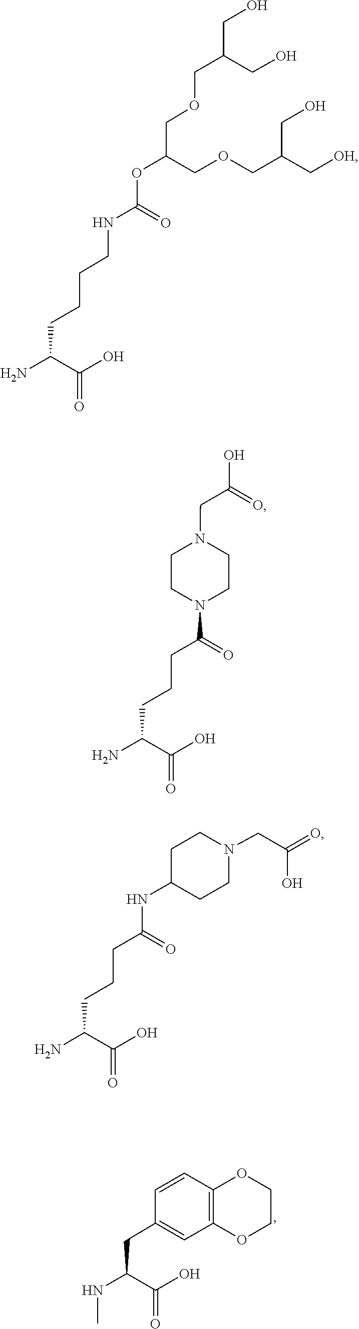

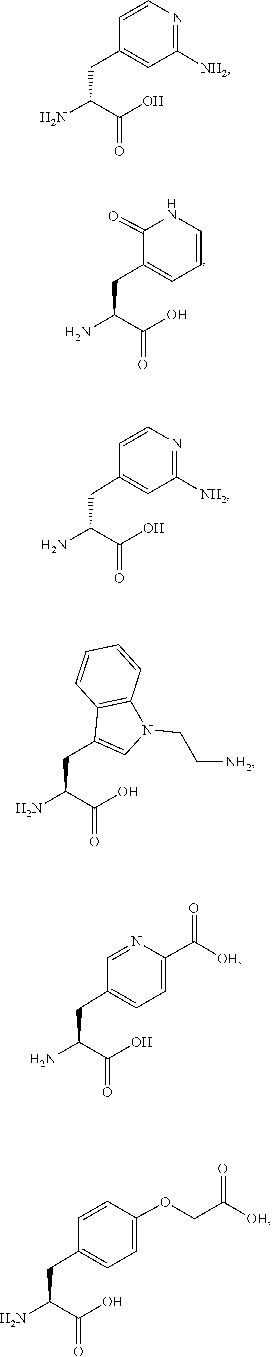 [SMILES: Nc1cc(C[C@@H](N)C(=O)O)ccn1 ; N[C@@H](Cc1ccc[nH]c1=O)C(=O)O ; Nc1cc(C[C@@H](N)C(=O)O)ccn1 ; NCCn1cc(C[C@H](N)C(=O)O)c2ccccc21 ; N[C@@H](Cc1ccc(C(=O)O)nc1)C(=O)O ; N[C@@H](Cc1ccc(OCC(=O)O)cc1)C(=O)O]

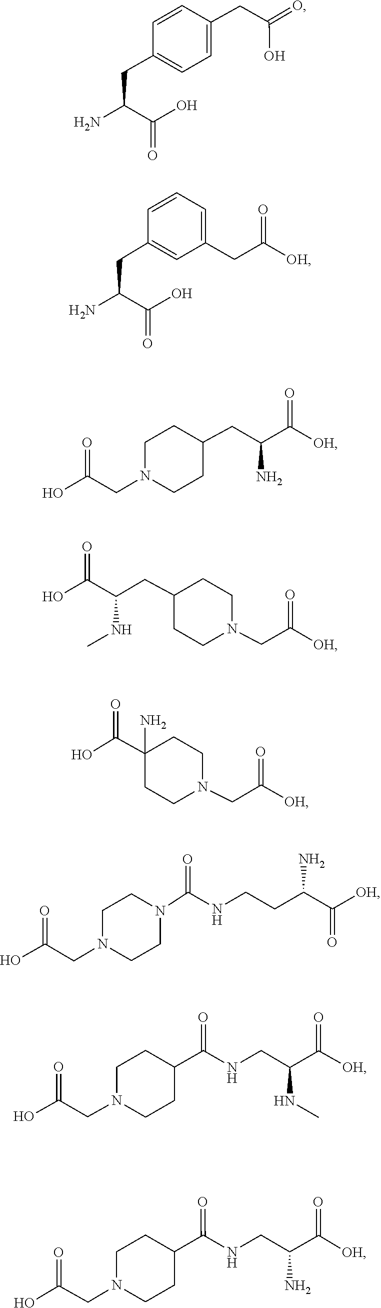

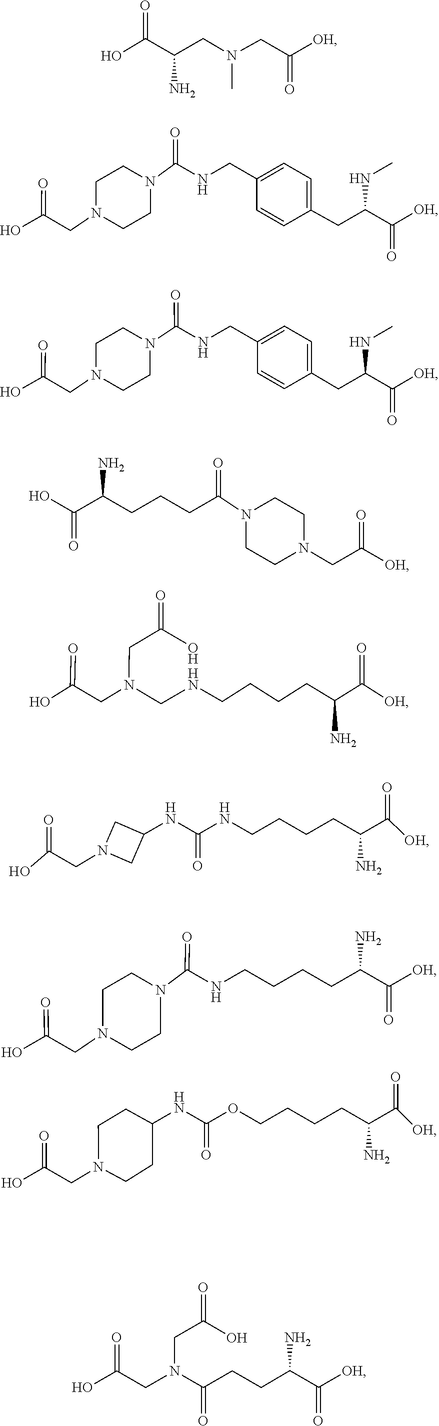

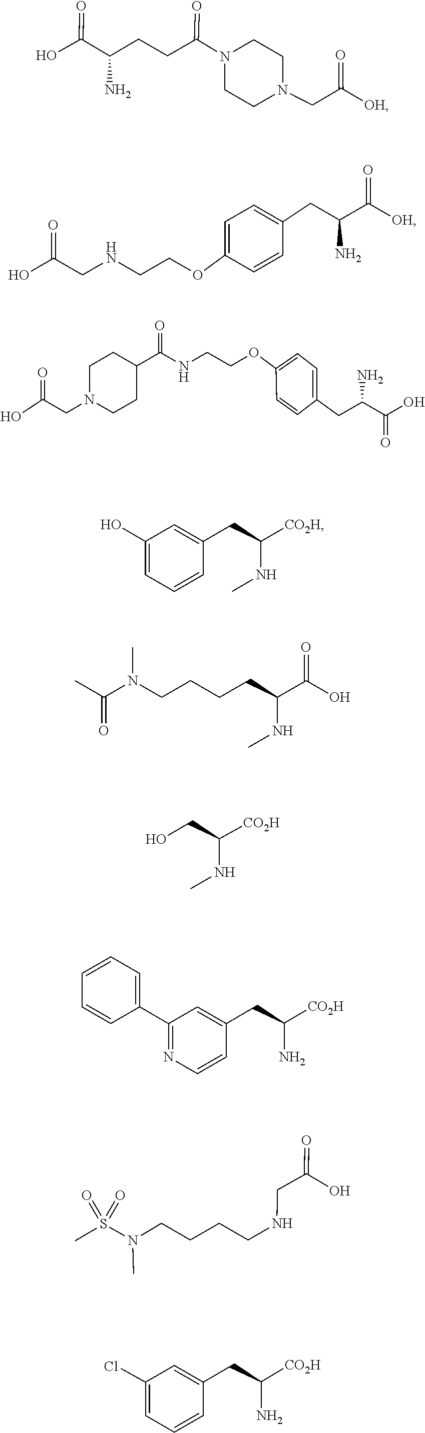

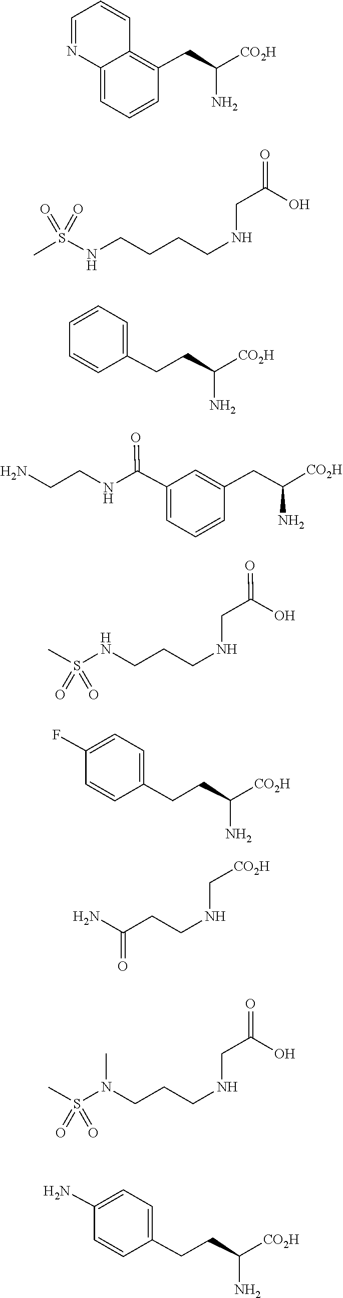

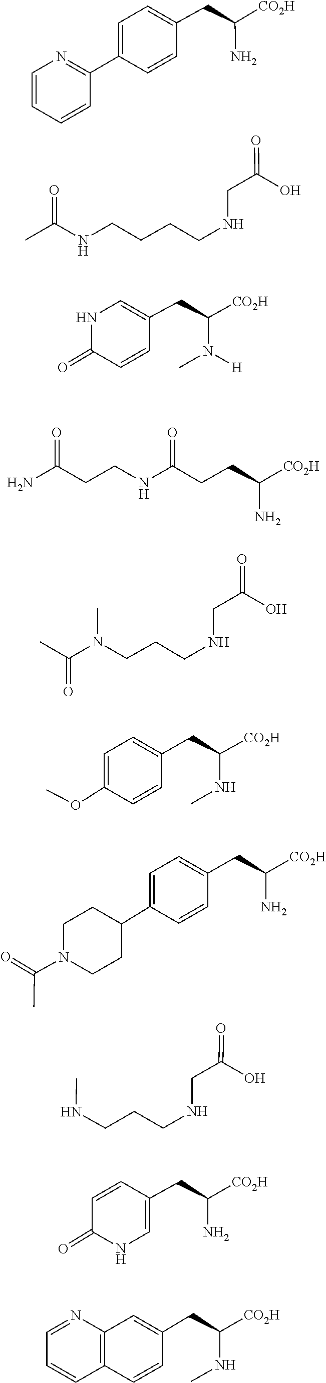

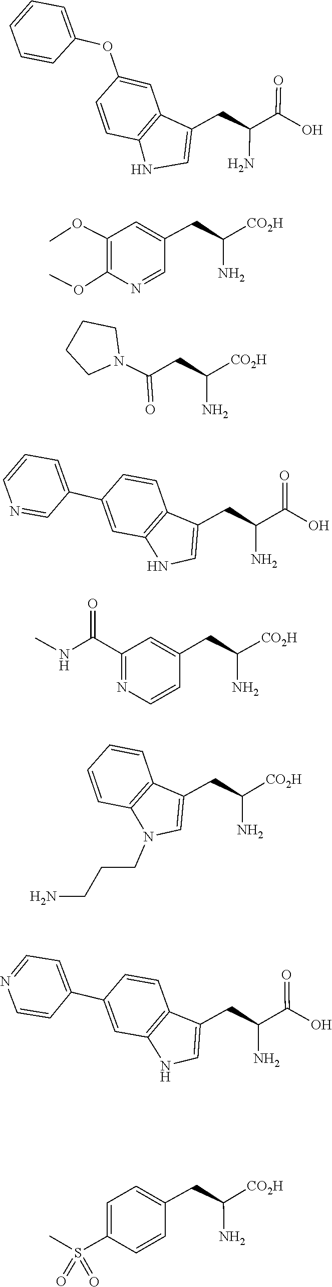

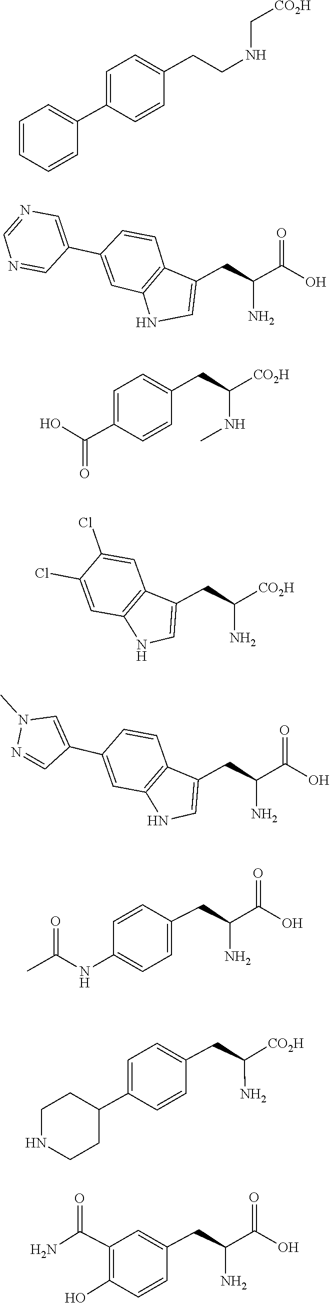

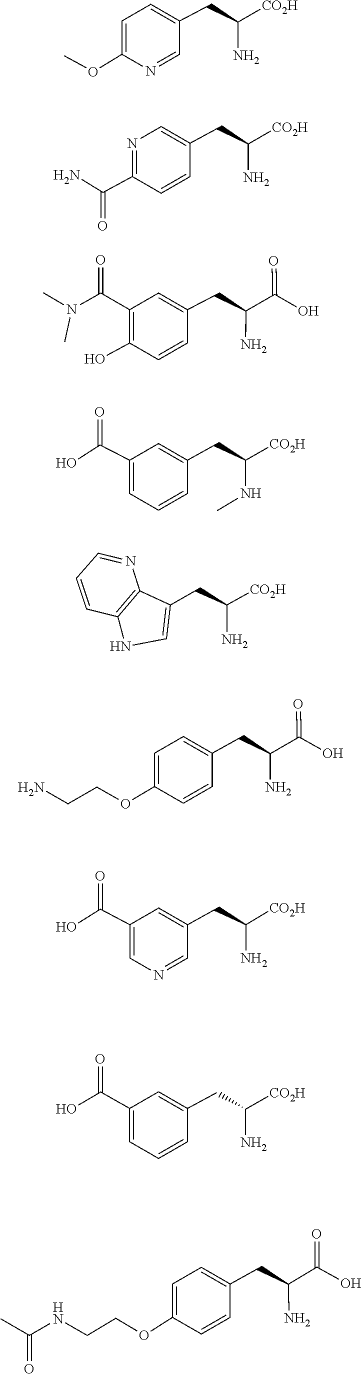

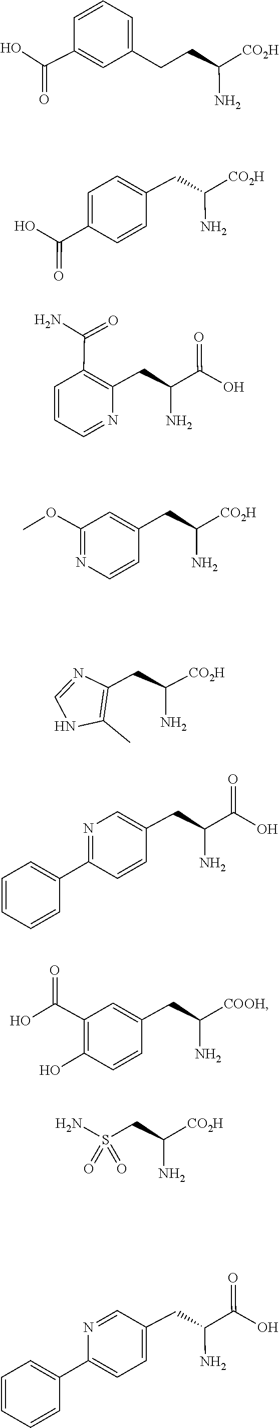

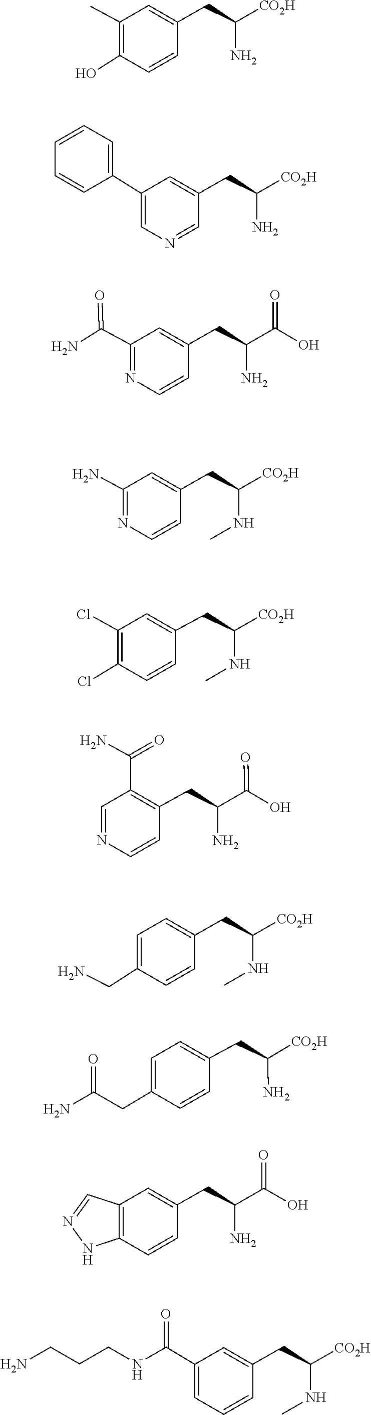

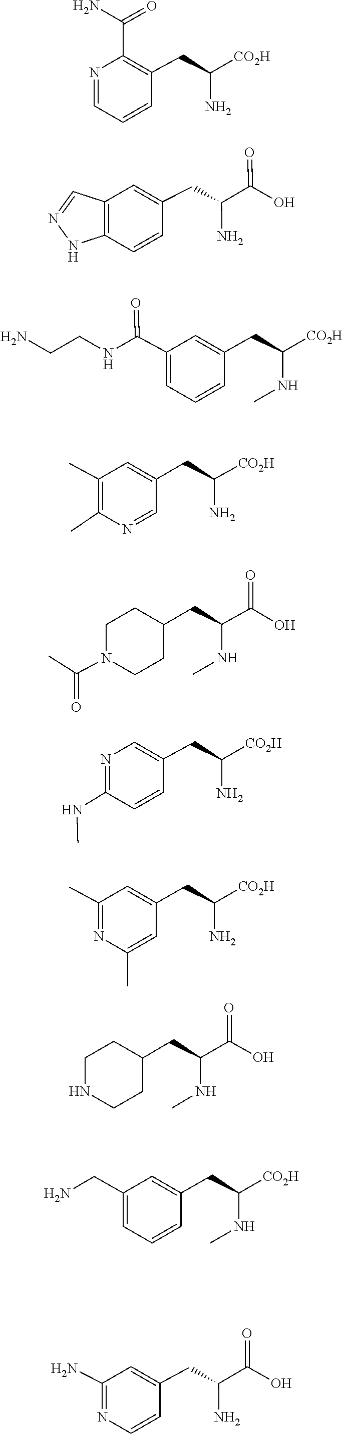

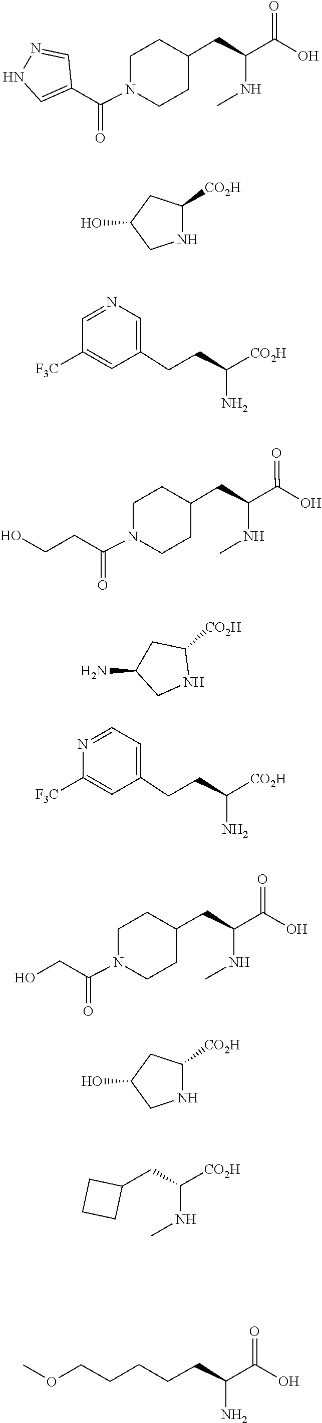

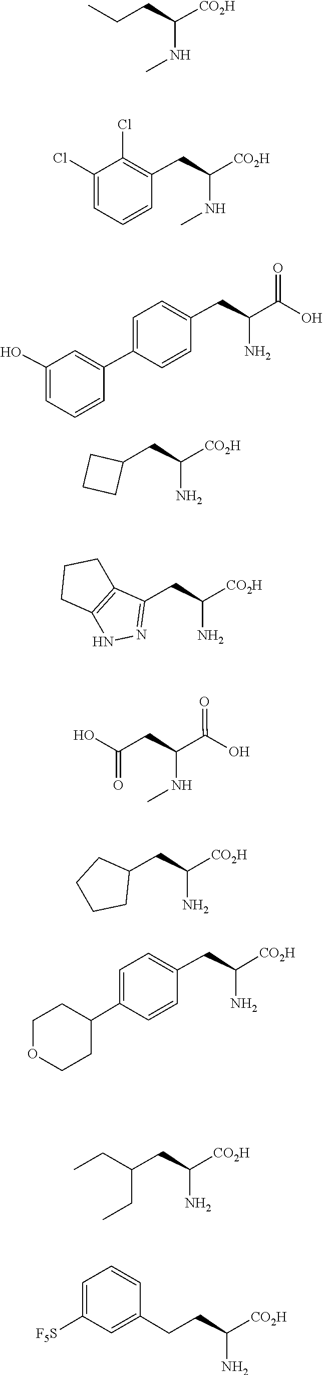

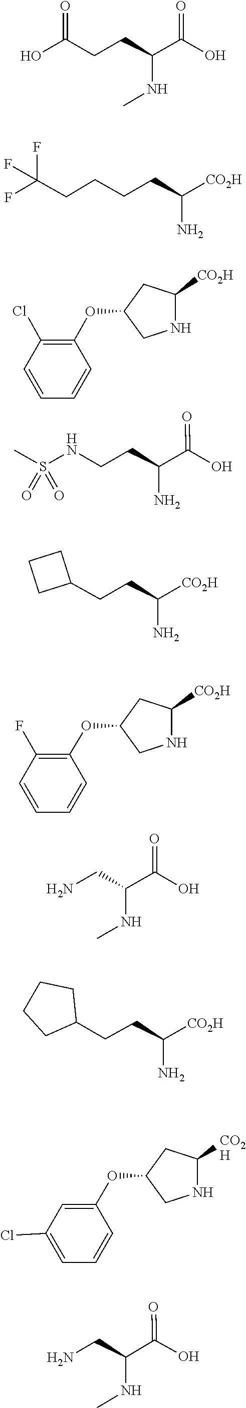

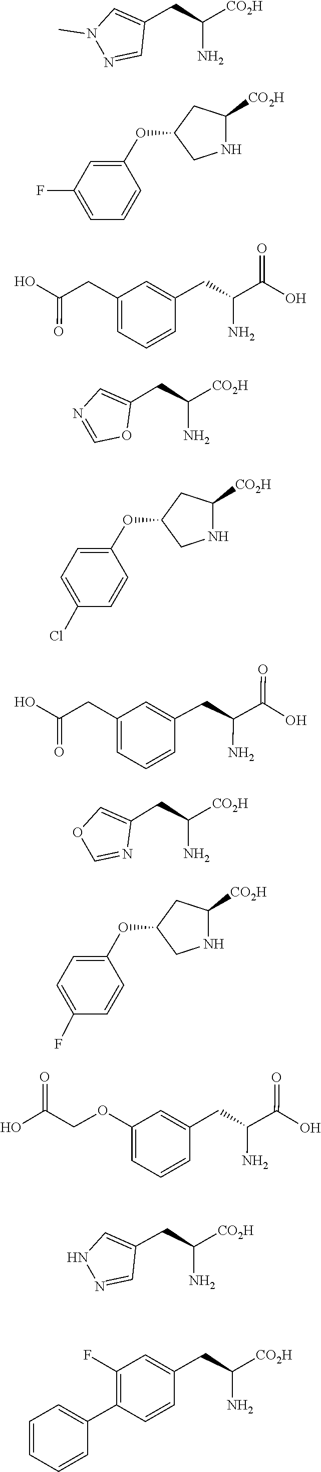

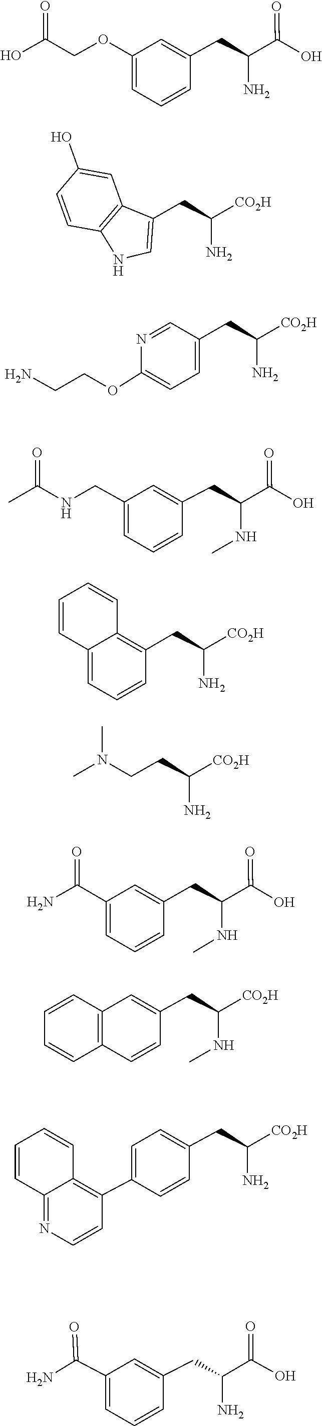

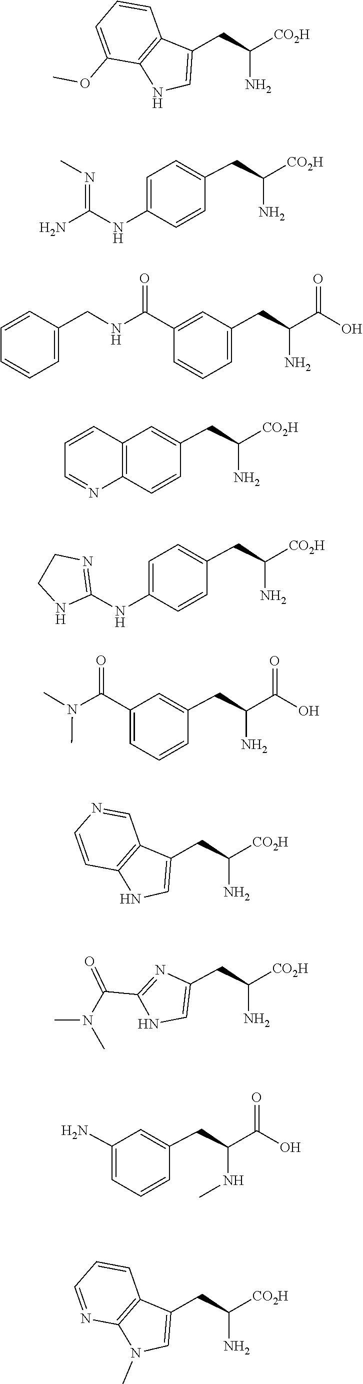

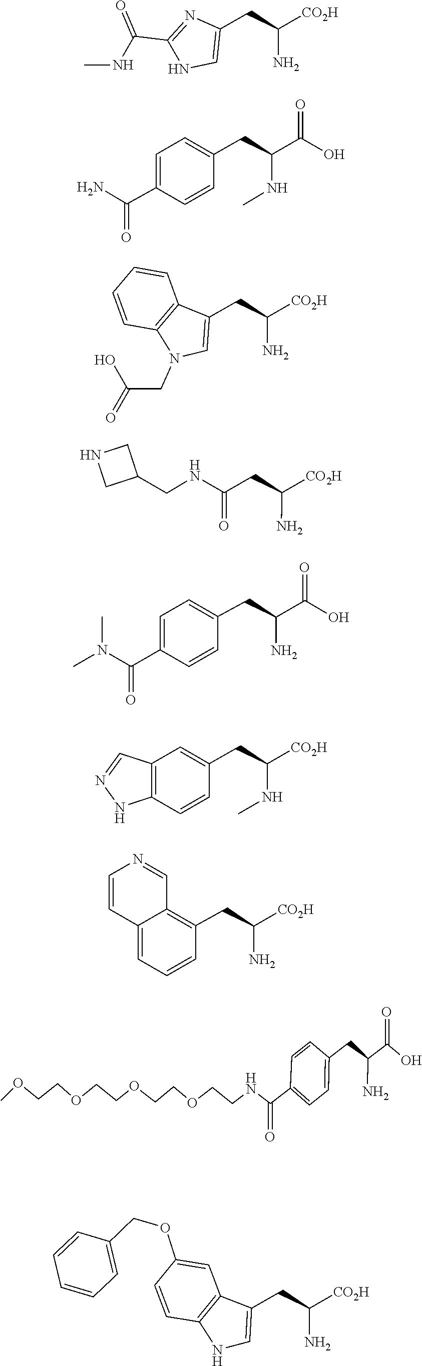

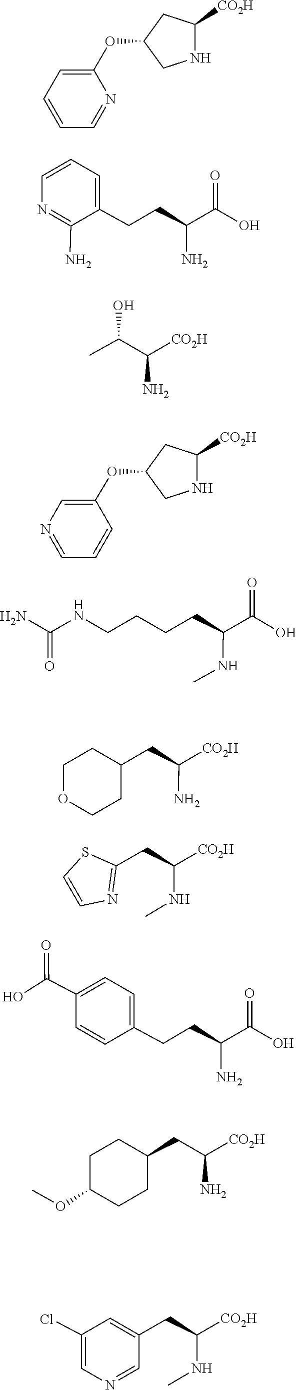

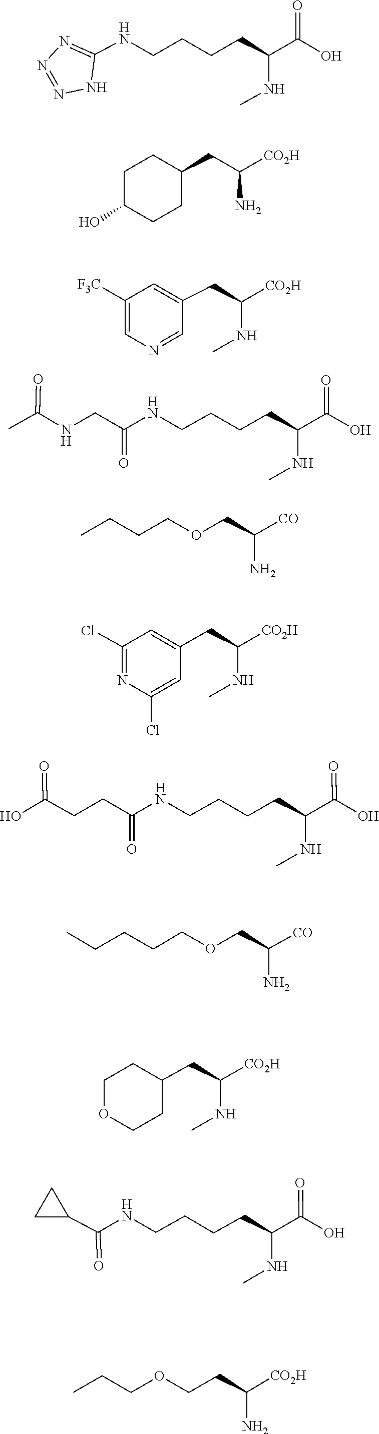

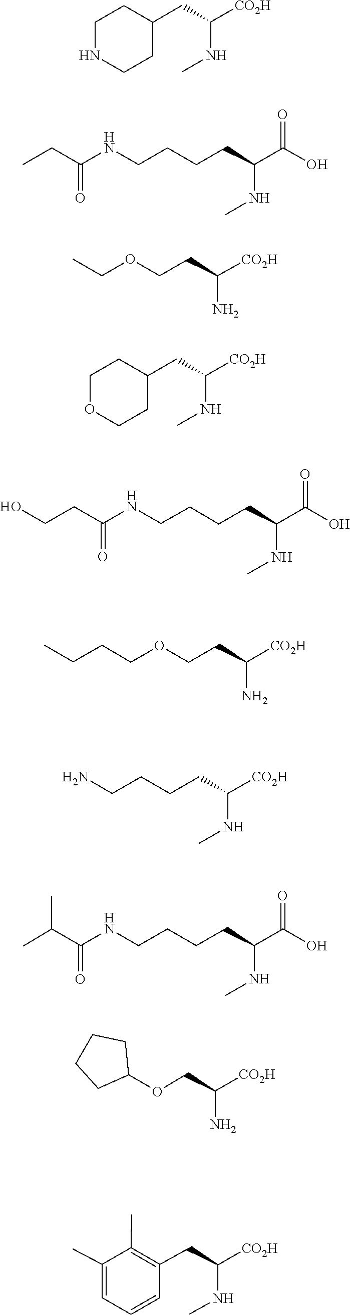

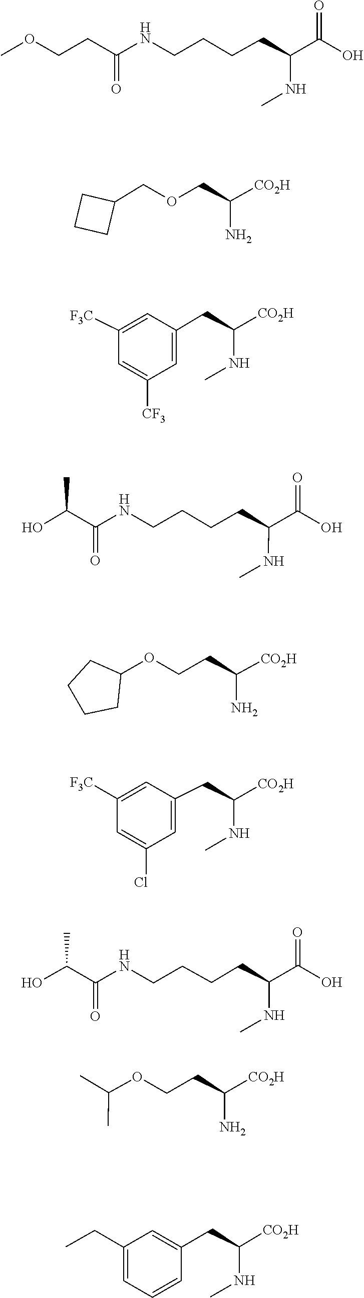

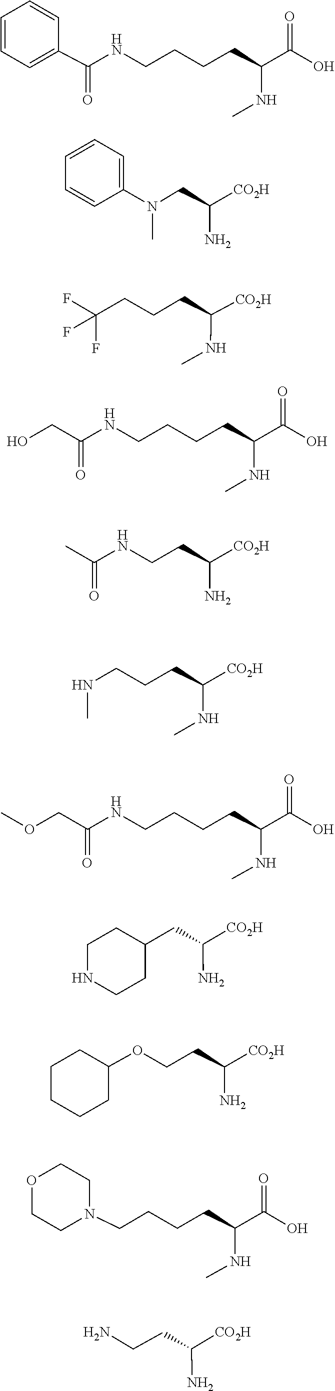

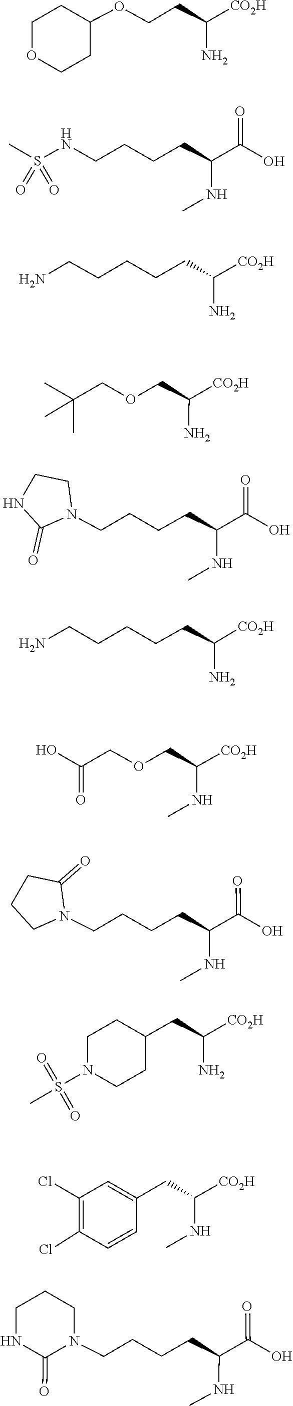

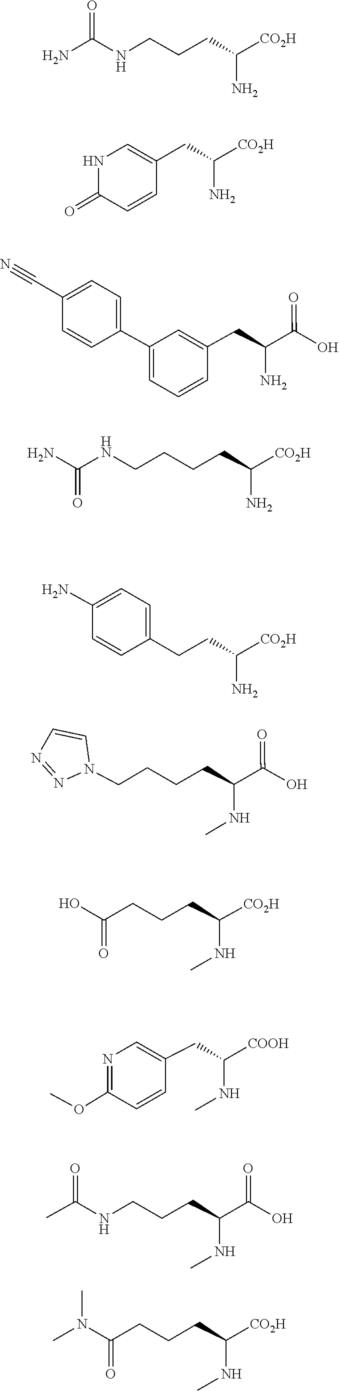

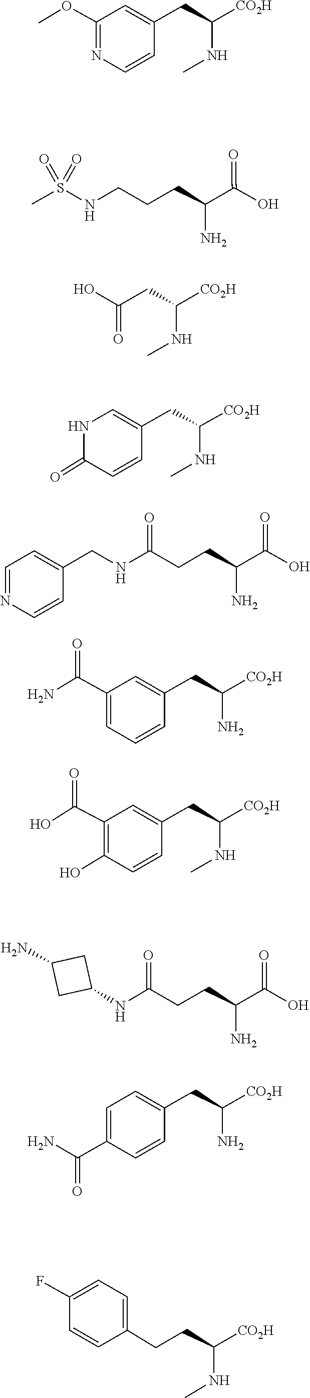

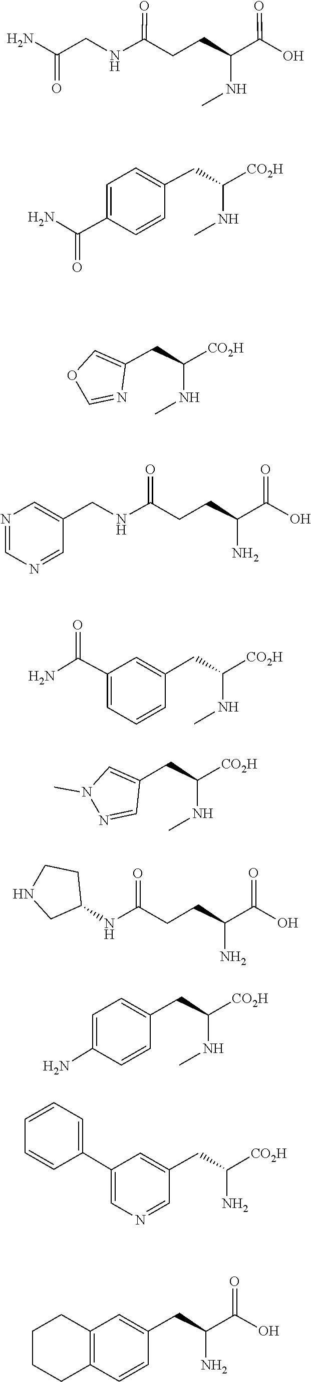

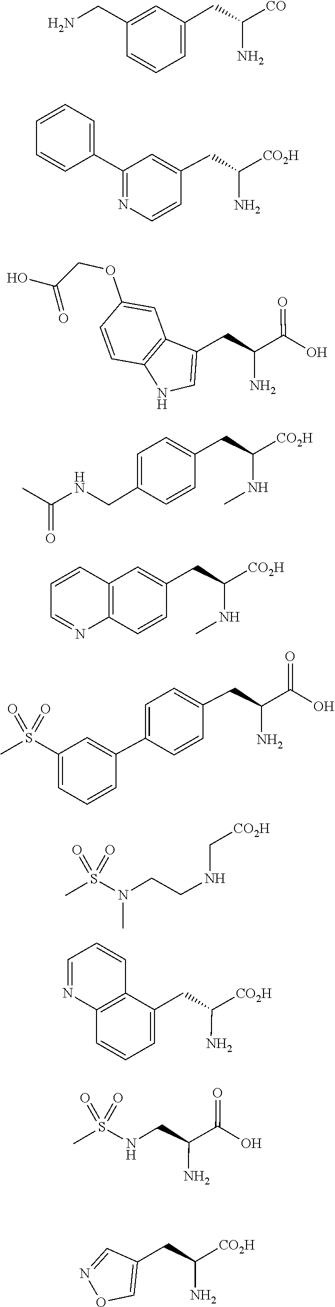

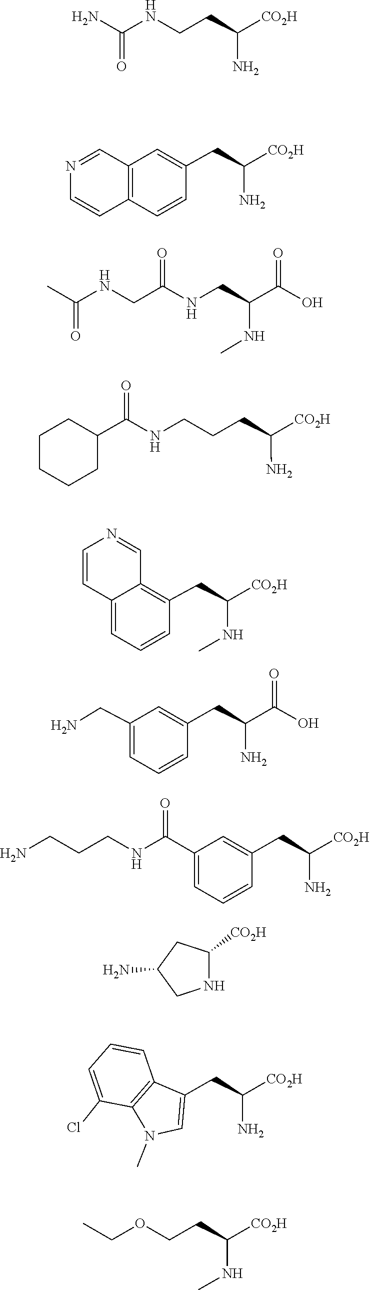

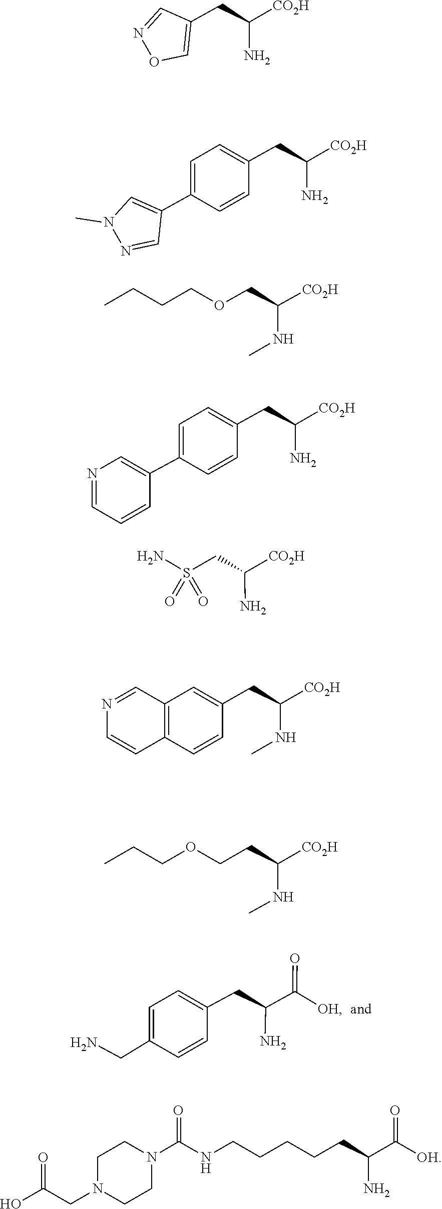

2. An amino acid derivative selected from the group consisting of 2-amino-6-[(4-methylpiperazine-1-carbonyl)amino]hexanoic acid, 6-acetamido-2-(methylamino)hexanoic acid, 2-amino-6-{[4-(2-hydroxyethyl)piperazine-1-carbonyl]amino}hexanoic acid, 2-amino-6-{[4-(carboxymethyl)piperazine-1-carbonyl]amino}hexanoic acid, 6-{[4-(carboxymethyl)piperazine-1-carbonyl]amino}-2-(methylamino)hexanoic acid, 2-amino-6-{[4-(carboxymethyl)piperazine-1-carbonyl]amino}hexanoic acid, 6-{[4-(carboxymethyl)piperazine-1-carbonyl]amino}-2-(methylamino)hexanoic acid, 6-amino-2-(methylamino)hexanoic acid, 2-amino-6-({methyl[(2S,3R,4R,5R)-2,3,4,5,6-pentahydroxyhexyl]carbamoyl}amino)hexanoic acid, 6-({methyl[(2S,3R,4R,5R)-2,3,4,5,6-pentahydroxyhexyl]carbamoyl}amino)-2-(methylamino)hexanoic acid, 2-amino-6-({methyl[(2S,3R,4R,5R)-2,3,4,5,6-pentahydroxyhexyl]carbamoyl}amino)hexanoic acid, 6-({methyl[(2S,3R,4R,5R)-2,3,4,5,6-pentahydroxyhexyl]carbamoyl}amino)-2-(methylamino)hexanoic acid, 3-amino-2-(methylamino)propanoic acid, 3-acetamido-2-(methylamino)propanoic acid, 2-amino-3-{[4-(carboxymethyl)piperazine-1-carbonyl]amino}propanoic acid, 3-{[4-(carboxymethyl)piperazine-1-carbonyl]amino}-2-(methylamino)propanoic acid, 2-amino-3-{[1-(carboxymethyl)piperidin-4-yl]formamido}propanoic acid, 4-amino-2-(methylamino)butanoic acid, 2-amino-2-[1-(carboxymethyl)piperidin-4-yl]acetic acid, 2-amino-3-[1-(carboxymethyl)piperidin-4-yl]propanoic acid, 2-amino-3-[1-(carboxymethyl)piperidin-4-yl]propanoic acid, (4R)-4-{[4-(carboxymethyl)piperazine-1-carbonyl]amino}pyrrolidine-2-carboxylic acid, (4S)-4-{[4-(carboxymethyl)piperazine-1-carbonyl]amino}pyrrolidine-2-carboxylic acid, 4-(2-amino-2-carboxyethyl)benzoic acid, 3-(2-amino-2-carboxyethyl)benzoic acid, 3-(3,4-dimethoxyphenyl)-2-(methylamino)propanoic acid, 3-(2H-1,3-benzodioxol-5-yl)-2-(methylamino)propanoic acid, 2-amino-3-(6-aminopyridin-3-yl)propanoic acid, 2-amino-3-(2-oxo-1,2-dihydropyridin-4-yl)propanoic acid, 4-(2-amino-2-carboxyethyl)benzoic acid, 3-(4-ethylphenyl)-2-(methylamino)propanoic acid, 3-(3,4-dimethylphenyl)-2-(methylamino)propanoic acid, 2-amino-3-(5-methyl-1H-imidazol-4-yl)propanoic acid, 2-amino-3-(1H-1,2,3-triazol-4-yl)propanoic acid, 2-amino-3-(1H-pyrazol-3-yl)propanoic acid, 2-amino-3-(1H-indol-6-yl)propanoic acid, 2-amino-3-{1H-pyrrolo[3,2-c]pyridin-3-yl}propanoic acid, 2-amino-3-(6-hydroxy-1H-indol-3-yl)propanoic acid, 2-(methylamino)-4-(pyridin-3-yl)butanoic acid, 2-(methylamino)-4-(pyridin-2-yl)butanoic acid, 4-(6-methoxypyridin-3-yl)-2-(methylamino)butanoic acid, 4-(5-methoxypyridin-2-yl)-2-(methylamino)butanoic acid, 2-amino-3-[3-({[4-(carboxymethyl)piperazine-1-carbonyl]amino}methyl)phenyl]propanoic acid, 2-amino-3-[4-({[4-(carboxymethyl)piperazine-1-carbonyl]amino}methyl)phenyl]propanoic acid, 2-amino-3-[4-({[4-(carboxymethyl)piperazine-1-carbonyl]amino}methyl)phenyl]propanoic acid, 2-amino-3-[4-(2-{[4-(carboxymethyl)piperazine-1-carbonyl]amino}ethoxy)phenyl]propanoic acid, 2-amino-3-(4-{2-[4-(carboxymethyl)piperazin-1-yl]-2-oxoethoxy}phenyl)propanoic acid, 2-amino-3-{6-[(carboxymethyl)amino]pyridin-3-yl}propanoic acid, 2-amino-6-({[1-(carboxymethyl)piperidin-4-yl]carbamoyl}amino)hexanoic acid, 2-amino-6-[({[1-(carboxymethyl)piperidin-4-yl]oxy}carbonyl)amino]hexanoic acid, 2-amino-6-{2-[1-(carboxymethyl)piperidin-4-yl]acetamido}hexanoic acid, 2-amino-6-[1-(carboxymethyl)piperidine-4-sulfonamido]hexanoic acid, 2-amino-6-[1-(carboxymethyl)piperidine-4-sulfonamido]hexanoic acid, 2-amino-6-({[(3R)-1-(carboxymethyl)pyrrolidin-3-yl]carbamoyl}amino)hexanoic acid, 6-[(5-amino-5-carboxypentyl)amino]pyridine-3-carboxylic acid, 2-amino-6-{[1-(carboxymethyl)piperidin-4-yl]formamido}hexanoic acid, 2-amino-6-[1-(carboxymethyl)piperidine-4-sulfonamido]hexanoic acid, 2-amino-6-{4-[(2-methoxyethoxy)methyl]-2-oxo-3,6,9-trioxa-1-azadecan-1-yl}hexanoic acid, 2-amino-6-{[({1,3-bis[(2,2-dimethyl-1,3-dioxan-5-yl)methoxy]propan-2-yl}oxy)carbonyl]amino}hexanoic acid, 2-amino-6-{[({1,3-bis[(2,2-dimethyl-1,3-dioxan-5-yl)methoxy]propan-2-yl}oxy)carbonyl]amino}hexanoic acid, 2-amino-6-{[({1,3-bis[3-hydroxy-2-(hydroxymethyl)propoxy]propan-2-yl}oxy)carbonyl]amino}hexanoic acid, 2-amino-6-[4-(carboxymethyl)piperazin-1-yl]-6-oxohexanoic acid, 2-amino-5-{[1-(carboxymethyl)piperidin-4-yl]carbamoyl}pentanoic acid, 3-(2,3-dihydro-1,4-benzodioxin-6-yl)-2-(methylamino)propanoic acid, 2-amino-3-(2-aminopyridin-4-yl)propanoic acid, 2-amino-3-(2-oxo-1,2-dihydropyridin-3-yl)propanoic acid, 2-amino-3-(2-aminopyridin-4-yl)propanoic acid, 2-amino-3-[1-(2-aminoethyl)-1H-indol-3-yl]propanoic acid, 5-(2-amino-2-carboxyethyl)pyridine-2-carboxylic acid, 2-amino-3-[4-(carboxymethoxy)phenyl]propanoic acid, 2-amino-3-[3-(carboxymethoxy)phenyl]propanoic acid, 2-amino-3-[4-(carboxymethyl)phenyl]propanoic acid, and 2-amino-3-[3-(carboxymethyl)phenyl]propanoic acid, (2S)-2-amino-6-{[bis(carboxymethyl)carbamoyl]amino}hexanoic acid, (2S)-2-amino-6-{[4-(carboxymethyl)piperazine-1-carbonyl]amino}hexanoic acid, (2S)-2-amino-5-[4-(carboxymethyl)piperazin-1-yl]-5-oxopentanoic acid, (2R)-2-amino-3-{[1-(carboxymethyl)piperidin-4-yl]formamido}propanoic acid, (2S)-3-{[1-(carboxymethyl)piperidin-4-yl]formamido}-2-(methylamino)propanoic acid, (2S)-2-amino-6-[4-(carboxymethyl)piperazin-1-yl]-6-oxohexanoic acid, (2S)-2-amino-4-[bis(carboxymethyl)carbamoyl]butanoic acid, (2S)-2-amino-3-[1-(carboxymethyl)piperidin-4-yl]propanoic acid, 4-amino-1-(carboxymethyl)piperidine-4-carboxylic acid, (2S)-3-[1-(carboxymethyl)piperidin-4-yl]-2-(methylamino)propanoic acid, (2S)-3-[4-({[4-(carboxymethyl)piperazine-1-carbonyl]amino}methyl)phenyl]-2-(methylamino)propanoic acid, (2R)-3-[4-({[4-(carboxymethyl)piperazine-1-carbonyl]amino}methyl)phenyl]-2-(methylamino)propanoic acid, (2S)-3-(3-hydroxyphenyl)-2-(methylamino)propanoic acid, (2S)-2-amino-3-[4-(2-{[1-(carboxymethyl)piperidin-4-yl]formamido}ethoxy)phenyl]propanoic acid, (2S)-2-amino-3-(4-{2-[(carboxymethyl)amino]ethoxy}phenyl)propanoic acid, (2R)-2-amino-6-({[1-(carboxymethyl)azetidin-3-yl]carbamoyl}amino)hexanoic acid, (2R)-2-amino-6-({[1-(carboxymethyl)piperidin-4-yl]carbamoyl}oxy)hexanoic acid, (2S)-2-amino-3-[(carboxymethyl)(methyl)amino]propanoic acid, and (2S)-2-amino-4-{[4-(carboxymethyl)piperazine-1-carbonyl]amino}butanoic acid, (2S)-2-(methylamino)-6-(N-methylacetamido)hexanoic acid, 2-{[4-(N-methylmethanesulfonamido)butyl]amino}acetic acid, 2-[(4-methanesulfonamidobutyl)amino]acetic acid, 2-[(3-methanesulfonamidopropyl)amino]acetic acid, 2-{[3-(N-methylmethanesulfonamido)propyl]amino}acetic acid, 2-[(4-acetamidobutyl)amino]acetic acid, 2-{[3-(N-methylacetamido)propyl]amino}acetic acid, 2-{[3-(methylamino)propyl]amino}acetic acid, (2S)-2-amino-3-(5-phenoxy-1H-indol-3-yl)propanoic acid, (2S)-2-amino-3-[6-(pyridin-3-yl)-1H-indol-3-yl]propanoic acid, (2S)-2-amino-3-[6-(pyridin-4-yl)-1H-indol-3-yl]propanoic acid, (2S)-2-amino-3-[6-(pyrimidin-5-yl)-1H-indol-3-yl]propanoic acid, (2S)-2-amino-3-[6-(1-methyl-1H-pyrazol-4-yl)-1H-indol-3-yl]propanoic acid, (2S)-2-amino-3-(3-carbamoyl-4-hydroxyphenyl)propanoic acid, (2S)-2-amino-3-[3-(dimethylcarbamoyl)-4-hydroxyphenyl]propanoic acid, (2S)-2-amino-3-[4-(2-aminoethoxy)phenyl]propanoic acid, (2S)-2-amino-3-[4-(2-acetamidoethoxy)phenyl]propanoic acid, (2S)-2-amino-3-(3-carbamoylpyridin-2-yl)propanoic acid, (2S)-2-amino-3-(6-phenylpyridin-3-yl)propanoic acid, (2R)-2-amino-3-(6-phenylpyridin-3-yl)propanoic acid, (2S)-2-amino-3-(2-carbamoylpyridin-4-yl)propanoic acid, (2S)-2-amino-3-(3-carbamoylpyridin-4-yl)propanoic acid, (2S)-2-amino-3-(1H-indazol-5-yl)propanoic acid, (2R)-2-amino-3-(1H-indazol-5-yl)propanoic acid, (2S)-3-(1-acetylpiperidin-4-yl)-2-(methylamino)propanoic acid, (2S)-2-(methylamino)-3-(piperidin-4-yl)propanoic acid, (2S)-2-(methylamino)-3-[1-(1H-pyrazole-4-carbonyl)piperidin-4-yl]propanoic acid, (2S)-3-[1-(3-hydroxypropanoyl)piperidin-4-yl]-2-(methylamino)propanoic acid, (2S)-3-[1-(2-hydroxyacetyl)piperidin-4-yl]-2-(methylamino)propanoic acid, (2S)-2-amino-7-methoxyheptanoic acid, (2S)-2-amino-3-{3′-hydroxy-[1,1′-biphenyl]-4-yl}propanoic acid, (2S)-2-(methylamino)butanedioic acid, (2S)-2-(methylamino)pentanedioic acid, (2S)-2-amino-4-methanesulfonamidobutanoic acid, (2R)-3-amino-2-(methylamino)propanoic acid, (2S)-3-amino-2-(methylamino)propanoic acid, (2R)-2-amino-3-[3-(carboxymethyl)phenyl]propanoic acid, (2S)-2-amino-3-[3-(carboxymethyl)phenyl]propanoic acid, (2R)-2-amino-3-[3-(carboxymethoxy)phenyl]propanoic acid, (2S)-2-amino-3-[3-(carboxymethoxy)phenyl]propanoic acid, (2S)-3-[3-(acetamidomethyl)phenyl]-2-(methylamino)propanoic acid, (2S)-3-(3-carbamoylphenyl)-2-(methylamino)propanoic acid, (2R)-2-amino-3-(3-carbamoylphenyl)propanoic acid, (2S)-2-amino-3-[3-(benzylcarbamoyl)phenyl]propanoic acid, (2S)-2-amino-3-[3-(dimethylcarbamoyl)phenyl]propanoic acid, (2S)-3-(3-aminophenyl)-2-(methylamino)propanoic acid, (2S)-3-(4-carbamoylphenyl)-2-(methylamino)propanoic acid, (2S)-2-amino-3-[4-(dimethylcarbamoyl)phenyl]propanoic acid, (2S)-2-amino-3-{4-[(2,5,8,11-tetraoxatridecan-13-yl)carbamoyl]phenyl}propanoic acid, (2S)-2-amino-4-(2-aminopyridin-3-yl)butanoic acid, (2S)-6-(carbamoylamino)-2-(methylamino)hexanoic acid, 4-[(3S)-3-amino-3-carboxypropyl]benzoic acid, (2S)-2-(methylamino)-6-[(1H-1,2,3,4-tetrazol-5-yl)amino]hexanoic acid, (2S)-6-(2-acetamidoacetamido)-2-(methylamino)hexanoic acid, (2S)-6-(3-carboxypropanamido)-2-(methylamino)hexanoic acid, (2S)-6-(cyclopropylformamido)-2-(methylamino)hexanoic acid, (2S)-2-(methylamino)-6-propanamidohexanoic acid, (2S)-6-(3-hydroxypropanamido)-2-(methylamino)hexanoic acid, (2S)-2-(methylamino)-6-(2-methylpropanamido)hexanoic acid, (2S)-6-(3-methoxypropanamido)-2-(methylamino)hexanoic acid, (2S)-6-[(2S)-2-hydroxypropanamido]-2-(methylamino)hexanoic acid, (2S)-6-[(2R)-2-hydroxypropanamido]-2-(methylamino)hexanoic acid, (2S)-2-(methylamino)-6-(phenylformamido)hexanoic acid, (2S)-6-(2-hydroxyacetamido)-2-(methylamino)hexanoic acid, (2S)-6-(2-methoxyacetamido)-2-(methylamino)hexanoic acid, (2S)-2-(methylamino)-6-(morpholin-4-yl)hexanoic acid, (2S)-6-methanesulfonamido-2-(methylamino)hexanoic acid, (2S)-2-(methylamino)-6-(2-oxoimidazolidin-1-yl)hexanoic acid, (2S)-2-(methylamino)-6-(2-oxopyrrolidin-1-yl)hexanoic acid, (2S)-2-(methylamino)-6-(2-oxo-1,3-diazinan-1-yl)hexanoic acid, (2S)-2-amino-3-{4′-cyano-[1,1′-biphenyl]-3-yl}propanoic acid, (2S)-2-(methylamino)-6-(1H-1,2,3-triazol-1-yl)hexanoic acid, (2S)-5-acetamido-2-(methylamino)pentanoic acid, (2S)-2-amino-5-methanesulfonamidopentanoic acid, (2S)-2-amino-4-{[(pyridin-4-yl)methyl]carbamoyl}butanoic acid, (2S)-2-amino-4-{[(1s,3s)-3-aminocyclobutyl]carbamoyl}butanoic acid, (2S)-4-[(carbamoylmethyl)carbamoyl]-2-(methylamino)butanoic acid, (2S)-2-amino-4-{[(pyrimidin-5-yl)methyl]carbamoyl}butanoic acid, (2S)-2-amino-4-{[(3S)-pyrrolidin-3-yl]carbamoyl}butanoic acid, (2S)-2-amino-3-(5,6,7,8-tetrahydronaphthalen-2-yl)propanoic acid, (2S)-2-amino-3-[5-(carboxymethoxy)-1H-indol-3-yl]propanoic acid, (2S)-2-amino-3-{3′-methanesulfonyl-[1,1′-biphenyl]-4-yl}propanoic acid, (2S)-2-amino-3-methanesulfonamidopropanoic acid, (2S)-3-(2-acetamidoacetamido)-2-(methylamino)propanoic acid, (2S)-2-amino-3-[3-(aminomethyl)phenyl]propanoic acid, (2S)-3-(4-aminophenyl)-2-(methylamino)propanoic acid, (S)-2-amino-3-(isoxazol-4-yl)propanoic acid, (2S)-3-hydroxy-2-(methylamino)propanoic acid, (S)-2-amino-3-(3-chlorophenyl)propanoic acid, (S)-2-amino-4-phenylbutanoic acid, (S)-2-amino-4-(4-fluorophenyl)butanoic acid, (S)-2-amino-4-(4-aminophenyl)butanoic acid, (S)-2-(methylamino)-3-(6-oxo-1,6-dihydropyridin-3-yl)propanoic acid, (S)-3-(4-methoxyphenyl)-2-(methylamino)propanoic acid, (S)-2-amino-3-(6-oxo-1,6-dihydropyridin-3-yl)propanoic acid, (S)-2-amino-3-(5,6-dimethoxypyridin-3-yl)propanoic acid, (S)-2-amino-3-(2-(methylcarbamoyl)pyridin-4-yl)propanoic acid, (S)-2-amino-3-(4-(methylsulfonyl)phenyl)propanoic acid, (S)-4-(2-carboxy-2-(methylamino)ethyl)benzoic acid, (S)-3-(4-acetamidophenyl)-2-aminopropanoic acid, (S)-2-amino-3-(6-methoxypyridin-3-yl)propanoic acid, (S)-3-(2-carboxy-2-(methylamino)ethyl)benzoic acid, (S)-5-(2-amino-2-carboxyethyl)nicotinic acid, (S)-3-(3-amino-3-carboxypropyl)benzoic acid, (S)-2-amino-3-(2-methoxy pyridin-4-yl)propanoic acid, (S)-5-(2-amino-2-carboxyethyl)-2-hydroxybenzoic acid, (S)-2-amino-3-(4-hydroxy-3-methylphenyl)propanoic acid, (S)-3-(2-aminopyridin-4-yl)-2-(methylamino)propanoic acid, (S)-3-(4-(aminomethyl)phenyl)-2-(methylamino)propanoic acid, (S)-3-(3-((3-aminopropyl)carbamoyl)phenyl)-2-(methylamino)propanoic acid, (S)-3-(3-((2-aminoethyl)carbamoyl)phenyl)-2-(methylamino)propanoic acid, (S)-2-amino-3-(6-(methylamino)pyridin-3-yl)propanoic acid, (S)-3-(3-(aminomethyl)phenyl)-2-(methylamino)propanoic acid, (2S,4R)-4-hydroxypyrrolidine-2-carboxylic acid, (2R,4S)-4-aminopyrrolidine-2-carboxylic acid, (2R,4R)-4-hydroxypyrrolidine-2-carboxylic acid, (S)-2-(methylamino)pentanoic acid, (S)-2-amino-3-cyclobutylpropanoic acid, (S)-2-amino-3-cyclopentylpropanoic acid, (S)-2-amino-4-ethylhexanoic acid, (S)-2-amino-7,7,7-trifluoroheptanoic acid, (S)-2-amino-4-cyclobutylbutanoic acid, (S)-2-amino-4-cyclopentylbutanoic acid, (S)-2-amino-3-(1-methyl-1H-pyrazol-4-yl)propanoic acid, (S)-2-amino-3-(oxazol-5-yl)propanoic acid, (S)-2-amino-3-(oxazol-4-yl)propanoic acid, (S)-2-amino-3-(1H-pyrazol-4-yl)propanoic acid, (S)-2-amino-3-(5-hydroxy-1H-indol-3-yl)propanoic acid, (S)-2-amino-3-(naphthalen-1-yl)propanoic acid, (S)-2-(methylamino)-3-(naphthalen-2-yl)propanoic acid, (S)-2-amino-3-(7-methoxy-1H-indol-3-yl)propanoic acid, (S)-2-amino-3-(quinolin-6-yl)propanoic acid, (S)-2-amino-3-(1H-pyrrolo[3,2-c]pyridin-3-yl)propanoic acid, (S)-2-amino-3-(1-methyl-1H-pyrrolo[2,3-b]pyridin-3-yl)propanoic acid, (2S)-2-amino-3-[1-(carboxymethyl)-1H-indol-3-yl]propanoic acid, (S)-3-(1H-indazol-5-yl)-2-(methylamino)propanoic acid, (S)-2-amino-3-(5-(benzyloxy)-1H-indol-3-yl)propanoic acid, (2S,3S)-2-amino-3-hydroxybutanoic acid, (S)-2-amino-3-(tetrahydro-2H-pyran-4-yl)propanoic acid, (S)-2-amino-3-((1r,4S)-4-methoxycyclohexyl)propanoic acid, (S)-2-amino-3-((1r,4S)-4-hydroxycyclohexyl)propanoic acid, (2S)-2-amino-3-butoxypropanoic acid, (2S)-2-amino-3-(pentyloxy)propanoic acid, (2S)-2-amino-4-propoxybutanoic acid, (2S)-2-amino-4-ethoxybutanoic acid, (2S)-2-amino-4-butoxybutanoic acid, (2S)-2-amino-3-(cyclopentyloxy)propanoic acid, (2S)-2-amino-3-(cyclobutylmethoxy)propanoic acid, (2S)-2-amino-4-(cyclopentyloxy)butanoic acid, (2S)-2-amino-4-(propan-2-yloxy)butanoic acid, (S)-2-amino-3-(methyl(phenyl)amino)propanoic acid, (S)-4-acetamido-2-aminobutanoic acid, (R)-2-amino-3-(piperidin-4-yl)propanoic acid, (R)-2,4-diaminobutanoic acid, (R)-2,7-diaminoheptanoic acid, (S)-2,7-diaminoheptanoic acid, (S)-2-amino-3-(1-(methylsulfonyl)piperidin-4-yl)propanoic acid, (R)-2-amino-5-ureidopentanoic acid, (2S)-2-amino-6-(carbamoylamino)hexanoic acid, (S)-2-(methylamino)hexanedioic acid, (S)-6-(dimethylamino)-2-(methylamino)-6-oxohexanoic acid, (2R)-2-(methylamino)butanedioic acid, (S)-2-amino-3-(3-carbamoylphenyl)propanoic acid, (S)-2-amino-3-(4-carbamoylphenyl)propanoic acid, (R)-3-(4-carbamoylphenyl)-2-(methylamino)propanoic acid, (R)-3-(3-carbamoylphenyl)-2-(methylamino)propanoic acid, (R)-2-amino-3-(3-(aminomethyl)phenyl)propanoic acid, (S)-3-(4-(acetamidomethyl)phenyl)-2-(methylamino)propanoic acid, 2-{[2-(N-methylmethanesulfonamido)ethyl]amino}acetic acid, (S)-2-amino-4-ureidobutanoic acid, (S)-2-amino-5-(cyclohexanecarboxamido)pentanoic acid, (S)-2-amino-3-(3-((3-aminopropyl)carbamoyl)phenyl)propanoic acid, (S)-2-amino-3-(7-chloro-1-methyl-1H-indol-3-yl)propanoic acid, (S)-2-amino-3-(4-(1-methyl-1H-pyrazol-4-yl)phenyl)propanoic acid, (S)-2-amino-3-(4-(pyridin-3-yl)phenyl)propanoic acid, (S)-3-(isoquinolin-7-yl)-2-(methylamino)propanoic acid, (S)-2-amino-3-(2-phenylpyridin-4-yl)propanoic acid, (S)-2-amino-3-(quinolin-5-yl)propanoic acid, (S)-2-amino-3-(3-((2-aminoethyl)carbamoyl)phenyl)propanoic acid, 2-[(2-carbamoylethyl)amino]acetic acid, (S)-2-amino-3-(4-(pyridin-2-yl)phenyl)propanoic acid, (2S)-2-amino-4-[(2-carbamoylethyl)carbamoyl]butanoic acid, (S)-3-(4-(1-acetylpiperidin-4-yl)phenyl)-2-aminopropanoic acid, (S)-2-(methylamino)-3-(quinolin-7-yl)propanoic acid, (S)-2-amino-4-oxo-4-(pyrrolidin-1-yl)butanoic acid, (2S)-2-amino-3-[1-(3-aminopropyl)-1H-indol-3-yl]propanoic acid, 2-[(2-{[1,1′-biphenyl]-4-yl}ethyl)amino]acetic acid, (S)-2-amino-3-(5,6-dichloro-1H-indol-3-yl)propanoic acid, (S)-2-amino-3-(4-(piperidin-4-yl)phenyl)propanoic acid, (S)-2-amino-3-(6-carbamoylpyridin-3-yl)propanoic acid, (S)-2-amino-3-(1H-pyrrolo[3,2-b]pyridin-3-yl)propanoic acid, (R)-3-(2-amino-2-carboxyethyl)benzoic acid, (R)-4-(2-amino-2-carboxyethyl)benzoic acid, (S)-2-amino-3-(5-methyl-1H-imidazol-4-yl)propanoic acid, (2R)-2-amino-3-sulfamoylpropanoic acid, (S)-2-amino-3-(5-phenylpyridin-3-yl)propanoic acid, (S)-3-(3,4-dichlorophenyl)-2-(methylamino)propanoic acid, (S)-2-amino-3-(4-(2-amino-2-oxoethyl)phenyl)propanoic acid, (S)-2-amino-3-(2-carbamoylpyridin-3-yl)propanoic acid, (S)-2-amino-3-(5,6-dimethylpyridin-3-yl)propanoic acid, (S)-2-amino-3-(2,6-dimethylpyridin-4-yl)propanoic acid, (R)-2-amino-3-(2-aminopyridin-4-yl)propanoic acid, (S)-2-amino-4-(5-(trifluoromethyl)pyridin-3-yl)butanoic acid, (S)-2-amino-4-(2-(trifluoromethyl)pyridin-4-yl)butanoic acid, (R)-3-cyclobutyl-2-(methylamino)propanoic acid, (S)-3-(2,3-dichlorophenyl)-2-(methylamino)propanoic acid, (S)-2-amino-3-(1,4,5,6-tetrahydrocyclopenta[c]pyrazol-3-yl)propanoic acid, (S)-2-amino-3-(4-(tetrahydro-2H-pyran-4-yl)phenyl)propanoic acid, (S)-2-amino-4-(3-(pentafluoro-16-sulfaneyl)phenyl)butanoic acid, (2S,4R)-4-(2-chlorophenoxy)pyrrolidine-2-carboxylic acid, (2S,4R)-4-(2-fluorophenoxy)pyrrolidine-2-carboxylic acid, (2S,4R)-4-(3-chlorophenoxy)pyrrolidine-2-carboxylic acid, (2S,4R)-4-(3-fluorophenoxy)pyrrolidine-2-carboxylic acid, (2S,4R)-4-(4-chlorophenoxy)pyrrolidine-2-carboxylic acid, (2S,4R)-4-(4-fluorophenoxy)pyrrolidine-2-carboxylic acid, (S)-2-amino-3-(2-fluoro-[1,1′-biphenyl]-4-yl)propanoic acid, (S)-2-amino-3-(6-(2-aminoethoxy)pyridin-3-yl)propanoic acid, (S)-2-amino-4-(dimethylamino)butanoic acid, (S)-2-amino-3-(4-(quinolin-4-yl)phenyl)propanoic acid, (S,E)-2-amino-3-(4-(2-methylguanidino)phenyl)propanoic acid, (S)-2-amino-3-(4-((4,5-dihydro-1H-imidazol-2-yl)amino)phenyl)propanoic acid, (S)-2-amino-3-(2-(dimethylcarbamoyl)-1H-imidazol-4-yl)propanoic acid, (S)-2-amino-3-(2-(methylcarbamoyl)-1H-imidazol-4-yl)propanoic acid, (2S)-2-amino-3-{[(azetidin-3-yl)methyl]carbamoyl}propanoic acid, (S)-2-amino-3-(isoquinolin-8-yl)propanoic acid, (2S,4R)-4-(pyridin-2-yloxy)pyrrolidine-2-carboxylic acid, (2S,4R)-4-(pyridin-3-yloxy)pyrrolidine-2-carboxylic acid, (S)-2-(methylamino)-3-(thiazol-2-yl)propanoic acid, (S)-3-(5-chloropyridin-3-yl)-2-(methylamino)propanoic acid, (S)-2-(methylamino)-3-(5-(trifluoromethyl)pyridin-3-yl)propanoic acid, (S)-3-(2,6-dichloropyridin-4-yl)-2-(methylamino)propanoic acid, (S)-2-(methylamino)-3-(tetrahydro-2H-pyran-4-yl)propanoic acid, (R)-2-(methylamino)-3-(piperidin-4-yl)propanoic acid, (R)-2-(methylamino)-3-(tetrahydro-2H-pyran-4-yl)propanoic acid, (2R)-6-amino-2-(methylamino)hexanoic acid, (S)-3-(2,3-dimethylphenyl)-2-(methylamino)propanoic acid, (S)-3-(3,5-bis(trifluoromethyl)phenyl)-2-(methylamino)propanoic acid, (S)-3-(3-chloro-5-(trifluoromethyl)phenyl)-2-(methylamino)propanoic acid, (S)-3-(3-ethylphenyl)-2-(methylamino)propanoic acid, (S)-6,6,6-trifluoro-2-(methylamino)hexanoic acid, (S)-2,5-bis(methylamino)pentanoic acid, (2S)-2-amino-4-(cyclohexyloxy)butanoic acid, (2S)-2-amino-4-(oxan-4-yloxy)butanoic acid, (2S)-2-amino-3-(2,2-dimethylpropoxy)propanoic acid, (2S)-3-(carboxymethoxy)-2-(methylamino)propanoic acid, (R)-3-(3,4-dichlorophenyl)-2-(methylamino)propanoic acid, (R)-2-amino-3-(6-oxo-1,6-dihydropyridin-3-yl)propanoic acid, (R)-2-amino-4-(4-aminophenyl)butanoic acid, (R)-3-(6-methoxypyridin-3-yl)-2-(methylamino)propanoic acid, (S)-3-(2-methoxypyridin-4-yl)-2-(methylamino)propanoic acid, (R)-2-(methylamino)-3-(6-oxo-1,6-dihydropyridin-3-yl)propanoic acid, (S)-5-(2-carboxy-2-(methylamino)ethyl)-2-hydroxybenzoic acid, (S)-4-(4-fluorophenyl)-2-(methylamino)butanoic acid, (S)-2-(methylamino)-3-(oxazol-4-yl)propanoic acid, (S)-3-(1-methyl-1H-pyrazol-4-yl)-2-(methylamino)propanoic acid, (R)-2-amino-3-(5-phenylpyridin-3-yl)propanoic acid, (R)-2-amino-3-(2-phenylpyridin-4-yl)propanoic acid, (S)-2-(methylamino)-3-(quinolin-6-yl)propanoic acid, (R)-2-amino-3-(quinolin-5-yl)propanoic acid, (S)-2-amino-3-(isoquinolin-7-yl)propanoic acid, (S)-3-(isoquinolin-8-yl)-2-(methylamino)propanoic acid, (2R,4R)-4-aminopyrrolidine-2-carboxylic acid, (2S)-4-ethoxy-2-(methylamino)butanoic acid, (2S)-3-butoxy-2-(methylamino)propanoic acid, (2S)-2-amino-3-sulfamoylpropanoic acid, (2S)-2-(methylamino)-4-propoxybutanoic acid, (2S)-2-amino-3-[4-(aminomethyl)phenyl]propanoic acid and (2S)-2-amino-7-{[4-(carboxymethyl)piperazine-1-carbonyl]amino}heptanoic acid.

3. The amino acid derivative according to claim 1, wherein one or more functional groups within the amino acid are protected by protecting groups.

4. An amino acid derivative according to claim 3, wherein the amino acid is selected from the group consisting of (2S)-2-[[(9H-fluoren-9-ylmethoxy)carbonyl]amino]-6-(4-methylpiperazine-1-carbonylamino)hexanoic acid hydrochloride, (2S)-6-({4-[2-(tert-butoxy)-2-oxoethyl]piperazine-1-carbonyl}amino)-2-({[(9H-fluoren-9-yl)methoxy]carbonyl}amino)hexanoic acid, (2S)-2-({[(9H-fluoren-9-yl)methoxy]carbonyl}amino)-6-({methyl[(2S,3R,4R,5R)-2,3,4,5-tetrahydroxy-6-{[tris(propan-2-yl)silyl]oxy}hexyl]carbamoyl}amino)hexanoic acid”, (2R)-6-({[(3R)-1-[2-(tert-butoxy)-2-oxoethyl]pyrrolidin-3-yl]carbamoyl}amino)-2-({[(9H-fluoren-9-yl)methoxy]carbonyl}amino)hexanoic acid, (2S)-6-[(4-{2-[(tert-butyldimethylsilyl)oxy]ethyl}piperazine-1-carbonyl)amino]-2-({[(9H-fluoren-9-yl)methoxy]carbonyl}amino)hexanoic acid, (2R)-6-({4-[2-(tert-butoxy)-2-oxoethyl]piperazine-1-carbonyl}amino)-2-({[(9H-fluoren-9-yl)methoxy]carbonyl}amino)hexanoic acid, (2S)-6-({4-[2-(tert-butoxy)-2-oxoethyl]piperazine-1-carbonyl}amino)-2-({[(9H-fluoren-9-yl)methoxy]carbonyl}(methyl)amino)hexanoic acid, (2R)-6-({4-[2-(tert-butoxy)-2-oxoethyl]piperazine-1-carbonyl}amino)-2-({[(9H-fluoren-9-yl)methoxy]carbonyl}(methyl)amino)hexanoic acid, (2S)-2-({[(9H-fluoren-9-yl)methoxy]carbonyl}(methyl)amino)-6-({methyl[(2S,3R,4R,5R)-2,3,4,5-tetrahydroxy-6-{[tris(propan-2-yl)silyl]oxy}hexyl]carbamoyl}amino)hexanoic acid, (2R)-2-({[(9H-fluoren-9-yl)methoxy]carbonyl}amino)-6-({methyl[(2S,3R,4R,5R)-2,3,4,5-tetrahydroxy-6-{[tris(propan-2-yl)silyl]oxy}hexyl]carbamoyl}amino)hexanoic acid, (2R)-6-[(4-{2-[(tert-butyldimethylsilyl)oxy]ethyl}piperazine-1-carbonyl)amino]-2-({[(9H-fluoren-9-yl)methoxy]carbonyl}amino)hexanoic acid, (2S)-3-({4-[2-(tert-butoxy)-2-oxoethyl]piperazine-1-carbonyl}amino)-2-({[(9H-fluoren-9-yl)methoxy]carbonyl}amino)propanoic acid, (2S)-3-({4-[2-(tert-butoxy)-2-oxoethyl]piperazine-1-carbonyl}amino)-2-({[(9H-fluoren-9-yl)methoxy]carbonyl}(methyl)amino)propanoic acid, (2S,4R)-4-({4-[2-(tert-butoxy)-2-oxoethyl]piperazine-1-carbonyl}amino)-1-{[(9H-fluoren-9-yl)methoxy]carbonyl}pyrrolidine-2-carboxylic acid, (2S,4S)-4-({4-[2-(tert-butoxy)-2-oxoethyl]piperazine-1-carbonyl}amino)-1-{[(9H-fluoren-9-yl)methoxy]carbonyl}pyrrolidine-2-carboxylic acid, (2S)-2-({[(9H-fluoren-9-yl)methoxy]carbonyl}amino)-6-[(4-{2-oxo-2-[(2-phenylpropan-2-yl)oxy]ethyl}piperazine-1-carbonyl)amino]hexanoic acid, (2S)-7-({4-[2-(tert-butoxy)-2-oxoethyl]piperazine-1-carbonyl}amino)-2-({[(9H-fluoren-9-yl)methoxy]carbonyl}amino)heptanoic acid, (2S)-3-{4-[2-({4-[2-(tert-butoxy)-2-oxoethyl]piperazine-1-carbonyl}amino)ethoxy]phenyl}-2-({[(9H-fluoren-9-yl)methoxy]carbonyl}amino)propanoic acid, (2R)-6-[({1-[2-(tert-butoxy)-2-oxoethyl]piperidin-4-yl}carbamoyl)amino]-2-({[(9H-fluoren-9-yl)methoxy]carbonyl}amino)hexanoic acid, (2R)-6-{[({1-[2-(tert-butoxy)-2-oxoethyl]piperidin-4-yl}oxy)carbonyl]amino}-2-({[(9H-fluoren-9-yl)methoxy]carbonyl}amino)hexanoic acid, (2S)-6-({1-[2-(tert-butoxy)-2-oxoethyl]piperidin-4-yl}formamido)-2-({[(9H-fluoren-9-yl)methoxy]carbonyl}amino)hexanoic acid, (2R)-6-{1-[2-(tert-butoxy)-2-oxoethyl]piperidine-4-sulfonamido}-2-({[(9H-fluoren-9-yl)methoxy]carbonyl}amino)hexanoic acid, (2S)-3-[4-(2-{4-[2-(tert-butoxy)-2-oxoethyl]piperazin-1-yl}-2-oxoethoxy)phenyl]-2-({[(9H-fluoren-9-yl)methoxy]carbonyl}amino)propanoic acid, (2R)-6-({1-[2-(tert-butoxy)-2-oxoethyl]piperidin-4-yl}formamido)-2-({[(9H-fluoren-9-yl)methoxy]carbonyl}amino)hexanoic acid, (2R)-6-(2-{1-[2-(tert-butoxy)-2-oxoethyl]piperidin-4-yl}acetamido)-2-({[(9H-fluoren-9-yl)methoxy]carbonyl}amino)hexanoic acid, (2S)-6-{1-[2-(tert-butoxy)-2-oxoethyl]piperidine-4-sulfonamido}-2-({[(9H-fluoren-9-yl)methoxy]carbonyl}amino)hexanoic acid, (2S)-3-({1-[2-(tert-butoxy)-2-oxoethyl]piperidin-4-yl}formamido)-2-({[(9H-fluoren-9-yl)methoxy]carbonyl}amino)propanoic acid, (2R)-6-{4-[2-(tert-butoxy)-2-oxoethyl]piperazin-1-yl}-2-({[(9H-fluoren-9-yl)methoxy]carbonyl}amino)-6-oxohexanoic acid, (2R)-5-({1-[2-(tert-butoxy)-2-oxoethyl]piperidin-4-yl}carbamoyl)-2-({[(9H-fluoren-9-yl)methoxy]carbonyl}amino)pentanoic acid, (2R)-6-{[({1,3-bis[(2,2-dimethyl-1,3-dioxan-5-yl)methoxy]propan-2-yl}oxy)carbonyl]amino}-2-({[(9H-fluoren-9-yl)methoxy]carbonyl}amino)hexanoic acid, (2R)-2-({[(9H-fluoren-9-yl)methoxy]carbonyl}amino)-6-{4-[(2-methoxyethoxy)methyl]-2-oxo-3,6,9-trioxa-1-azadecan-1-yl}hexanoic acid, (2S)-6-{[({1,3-bis[(2,2-dimethyl-1,3-dioxan-5-yl)methoxy]propan-2-yl}oxy)carbonyl]amino}-2-({[(9H-fluoren-9-yl)methoxy]carbonyl}amino)hexanoic acid, (2S)-6-({5-[(tert-butoxy)carbonyl]pyridin-2-yl}amino)-2-({[(9H-fluoren-9-yl)methoxy]carbonyl}amino)hexanoic acid, (2S)-2-({[(9H-fluoren-9-yl)methoxy]carbonyl}(methyl)amino)-6-{[(prop-2-en-1-yloxy)carbonyl]amino}hexanoic acid, “(2S)-6-acetamido-2-({[(9H-fluoren-9-yl)methoxy]carbonyl}(methyl)amino)hexanoic acid, (2S)-2-({[(9H-fluoren-9-yl)methoxy]carbonyl}(methyl)amino)-4-{[(prop-2-en-1-yloxy)carbonyl]amino}butanoic acid, (2S)-3-acetamido-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-{[(prop-2-en-1-yloxy)carbonyl]amino}propanoic acid, (2S)-3-{1-[2-(tert-butoxy)-2-oxoethyl]piperidin-4-yl}-2-({[(9H-fluoren-9-yl)methoxy]carbonyl}amino)propanoic acid, (2S)-2-{1-[2-(tert-butoxy)-2-oxoethyl]piperidin-4-yl}-2-({[(9H-fluoren-9-yl)methoxy]carbonyl}amino)acetic acid, (2R)-3-{1-[2-(tert-butoxy)-2-oxoethyl]piperidin-4-yl}-2-({[(9H-fluoren-9-yl)methoxy]carbonyl}amino)propanoic acid, (2S)-3-{4-[({4-[2-(tert-butoxy)-2-oxoethyl]piperazine-1-carbonyl}amino)methyl]phenyl}-2-({[(9H-fluoren-9-yl)methoxy]carbonyl}amino)propanoic acid, (2R)-3-{4-[({4-[2-(tert-butoxy)-2-oxoethyl]piperazine-1-carbonyl}amino)methyl]phenyl}-2-({[(9H-fluoren-9-yl)methoxy]carbonyl}amino)propanoic acid, (2S)-3-{3-1[({4-[2-(tert-butoxy)-2-oxoethyl]piperazine-1-carbonyl}amino)methyl]phenyl}-2-({[(9H-fluoren-9-yl)methoxy]carbonyl}amino)propanoic acid, (2S)-3-(6-{[2-(tert-butoxy)-2-oxoethyl]amino}pyridin-3-yl)-2-({[(9H-fluoren-9-yl)methoxy]carbonyl}amino)propanoic acid, (2R)-3-{3-[(tert-butoxy)carbonyl]phenyl}-2-({[(9H-fluoren-9-yl)methoxy]carbonyl}amino)propanoic acid, (2S)-3-{1-[(tert-butoxy)carbonyl]-1H-pyrrolo[3,2-c]pyridin-3-yl}-2-({[(9H-fluoren-9-yl)methoxy]carbonyl}amino)propanoic acid, (2S)-2-({[(9H-fluoren-9-yl)methoxy]carbonyl}amino)-3-[1-(triphenylmethyl)-1H-pyrazol-3-yl]propanoic acid, (2S)-3-(3,4-dimethylphenyl)-2-({[(9H-fluoren-9-yl)methoxy]carbonyl}(methyl)amino)propanoic acid, (2S)-3-(3,4-dimethoxyphenyl)-2-({[(9H-fluoren-9-yl)methoxy]carbonyl}(methyl)amino)propanoic acid, (2S)-3-{1-[(tert-butoxy)carbonyl]-1H-indol-6-yl}-2-({[(9H-fluoren-9-yl)methoxy]carbonyl}amino)propanoic acid, (2S)-3-{1-[(tert-butoxy)carbonyl]-5-methyl-1H-imidazol-4-yl}-2-({[(9H-fluoren-9-yl)methoxy]carbonyl}amino)propanoic acid, (2S)-2-({[(9H-fluoren-9-yl)methoxy]carbonyl}amino)-3-(2-oxo-1,2-dihydropyridin-4-yl)propanoic acid, (2R)-3-(6-{[(tert-butoxy)carbonyl]amino}pyridin-3-yl)-2-({[(9H-fluoren-9-yl)methoxy]carbonyl}amino)propanoic acid, (2S)-2-({[(9H-fluoren-9-yl)methoxy]carbonyl}amino)-3-(6-hydroxy-1H-indol-3-yl)propanoic acid, (2S)-3-(2H-1,3-benzodioxol-5-yl)-2-({[(9H-fluoren-9-yl)methoxy]carbonyl}(methyl)amino)propanoic acid, (2S)-3-(4-ethylphenyl)-2-({[(9H-fluoren-9-yl)methoxy]carbonyl}(methyl)amino)propanoic acid, (2S)-2-({[(9H-fluoren-9-yl)methoxy]carbonyl}(methyl)amino)-4-(pyridin-3-yl)butanoic acid, (2S)-2-({[(9H-fluoren-9-yl)methoxy]carbonyl}(methyl)amino)-4-(pyridin-2-yl)butanoic acid, (2S)-2-({[(9H-fluoren-9-yl)methoxy]carbonyl}(methyl)amino)-4-(5-methoxypyridin-2-yl)butanoic acid, (2S)-2-({[(9H-fluoren-9-yl)methoxy]carbonyl}(methyl)amino)-4-(6-methoxypyridin-3-yl)butanoic acid, (2S)-2-({[(9H-fluoren-9-yl)methoxy]carbonyl}amino)-3-(1H-1,2,3-triazol-4-yl)propanoic acid, (2S)-2-({[(9H-fluoren-9-yl)methoxy]carbonyl}amino)-3-{4-[(prop-2-en-1-yloxy)carbonyl]phenyl}propanoic acid, (2S)-3-{6-[(tert-butoxy)carbonyl]pyridin-3-yl}-2-({[(9H-fluoren-9-yl)methoxy]carbonyl}amino)propanoic acid, (2S)-6-({bis[2-(tert-butoxy)-2-oxoethyl]carbamoyl}amino)-2-({[(9H-fluoren-9-yl)methoxy]carbonyl}amino)hexanoic acid, (2S)-2-({[(9H-fluoren-9-yl)methoxy]carbonyl}amino)-6-({4-[2-oxo-2-(prop-2-en-1-yloxy)ethyl]piperazine-1-carbonyl}amino)hexanoic acid, (2S)-2-({[(9H-fluoren-9-yl)methoxy]carbonyl}amino)-6[(4-{2-[(3-methylpentan-3-yl)oxy]-2-oxoethyl}piperazine-1-carbonyl)amino]hexanoic acid, (2S)-5-{4-[2-(tert-butoxy)-2-oxoethyl]piperazin-1-yl}-2-({[(9H-fluoren-9-yl)methoxy]carbonyl}amino)-5-oxopentanoic acid, (2R)-3-({1-[2-(tert-butoxy)-2-oxoethyl]piperidin-4-yl}formamido)-2-({[(9H-fluoren-9-yl)methoxy]carbonyl}amino)propanoic acid, (2S)-3-({1-[2-(tert-butoxy)-2-oxoethyl]piperidin-4-yl}formamido)-2-({[(9H-fluoren-9-yl)methoxy]carbonyl}(methyl)amino)propanoic acid, (2S)-6-{4-[2-(tert-butoxy)-2-oxoethyl]piperazin-1-yl}-2-({[(9H-fluoren-9-yl)methoxy]carbonyl}amino)-6-oxohexanoic acid, (2S)-4-{bis[2-(tert-butoxy)-2-oxoethyl]carbamoyl}-2-({[(9H-fluoren-9-yl)methoxy]carbonyl}amino)butanoic acid, (2S)-2-({[(9H-fluoren-9-yl)methoxy]carbonyl}amino)-3-{1-[2-oxo-2-(prop-2-en-1-yloxy)ethyl]piperidin-4-yl}propanoic acid, 1-[2-(tert-butoxy)-2-oxoethyl]-4-({[(9H-fluoren-9-yl)methoxy]carbonyl}amino)piperidine-4-carboxylic acid, (2S)-3-{1-[2-(tert-butoxy)-2-oxoethyl]piperidin-4-yl}-2-({[(9H-fluoren-9-yl)methoxy]carbonyl}(methyl)amino)propanoic acid, (2R)-3-{4-[({4-[2-(tert-butoxy)-2-oxoethyl]piperazine-1-carbonyl}amino)methyl]phenyl}-2-({[(9H-fluoren-9-yl)methoxy]carbonyl}(methyl)amino)propanoic acid, (2R)-3-{4-[({4-[2-(tert-butoxy)-2-oxoethyl]piperazine-1-carbonyl}amino)methyl]phenyl}-2-({[(9H-fluoren-9-yl)methoxy]carbonyl}(methyl)amino)propanoic acid, (2S)-3-[3-(tert-butoxy)phenyl]-2-({[(9H-fluoren-9-yl)methoxy]carbonyl}(methyl)amino)propanoic acid, (2S)-3-{4-[2-({1-[2-(tert-butoxy)-2-oxoethyl]piperidin-4-yl}formamido)ethoxy]phenyl}-2-({[(9H-fluoren-9-yl)methoxy]carbonyl}amino)propanoic acid, (2S)-3-[4-(2-{[2-(tert-butoxy)-2-oxoethyl][(tert-butoxy)carbonyl]amino}ethoxy)phenyl]-2-({[(9H-fluoren-9-yl)methoxy]carbonyl}amino)propanoic acid, (2R)-6-[({1-[2-(tert-butoxy)-2-oxoethyl]azetidin-3-yl}carbamoyl)amino]-2-({[(9H-fluoren-9-yl)methoxy]carbonyl}amino)hexanoic acid, (2R)-6-[({1-[2-(tert-butoxy)-2-oxoethyl]piperidin-4-yl}carbamoyl)oxy]-2-({[(9H-fluoren-9-yl)methoxy]carbonyl}amino)hexanoic acid, (S)-2-((((9H-fluoren-9-yl)methoxy)carbonyl)amino)-3-((2-(tert-butoxy)-2-oxoethyl)(methyl)amino)propanoic acid, (2S)-2-({[(9H-fluoren-9-yl)methoxy]carbonyl}amino)-3-{methyl[2-oxo-2-(prop-2-en-1-yloxy)ethyl]amino}propanoic acid, and (2S)-4-({4-[2-(tert-butoxy)-2-oxoethyl]piperazine-1-carbonyl}amino)-2-({[(9H-fluoren-9-yl)methoxy]carbonyl}amino)butanoic acid, (2S)-2-({[(9H-fluoren-9-yl)methoxy]carbonyl}(methyl)amino)-6-(N-methylacetamido)hexanoic acid, 2-({[(9H-fluoren-9-yl)methoxy]carbonyl}[4-(N-methylmethanesulfonamido)butyl]amino)acetic acid, 2-({[(9H-fluoren-9-yl)methoxy]carbonyl}(4-methanesulfonamidobutyl)amino)acetic acid, 2-({[(9H-fluoren-9-yl)methoxy]carbonyl}(3-methanesulfonamidopropyl)amino)acetic acid, 2-({[(9H-fluoren-9-yl)methoxy]carbonyl}[3-(N-methylmethanesulfonamido)propyl]amino)acetic acid, 2-[(4-acetamidobutyl)({[(9H-fluoren-9-yl)methoxy]carbonyl})amino]acetic acid, 2-({[(9H-fluoren-9-yl)methoxy]carbonyl}[3-(N-methylacetamido)propyl]amino)acetic acid, 2-[(3-{[(tert-butoxy)carbonyl](methyl)amino}propyl)({[(9H-fluoren-9-yl)methoxy]carbonyl})amino]acetic acid, (2S)-3-{1-[(tert-butoxy)carbonyl]-5-phenoxy-1H-indol-3-yl}-2-({[(9H-fluoren-9-yl)methoxy]carbonyl}amino)propanoic acid, (2S)-2-({[(9H-fluoren-9-yl)methoxy]carbonyl}amino)-3-[6-(pyridin-3-yl)-1H-indol-3-yl]propanoic acid, (2S)-2-({[(9H-fluoren-9-yl)methoxy]carbonyl}amino)-3-[6-(pyridin-4-yl)-1H-indol-3-yl]propanoic acid, (2S)-2-({[(9H-fluoren-9-yl)methoxy]carbonyl}amino)-3-[6-(pyrimidin-5-yl)-1H-indol-3-yl]propanoic acid, (2S)-3-{1-[(tert-butoxy)carbonyl]-6-(1-methyl-1H-pyrazol-4-yl)-1H-indol-3-yl}-2-({[(9H-fluoren-9-yl)methoxy]carbonyl}amino)propanoic acid, (2S)-3-(3-carbamoyl-4-hydroxyphenyl)-2-({[(9H-fluoren-9-yl)methoxy]carbonyl}amino)propanoic acid, (2S)-3-[3-(dimethylcarbamoyl)-4-hydroxyphenyl]-2-({[(9H-fluoren-9-yl)methoxy]carbonyl}amino)propanoic acid, (2S)-2-({[(9H-fluoren-9-yl)methoxy]carbonyl}amino)-3-[4-(2-{[(prop-2-en-1-yloxy)carbonyl]amino}ethoxy)phenyl]propanoic acid, (2S)-3-[4-(2-acetamidoethoxy)phenyl]-2-({[(9H-fluoren-9-yl)methoxy]carbonyl}amino)propanoic acid, (2S)-3-(3-carbamoylpyridin-2-yl)-2-({[(9H-fluoren-9-yl)methoxy]carbonyl}amino)propanoic acid, (2S)-2-({[(9H-fluoren-9-yl)methoxy]carbonyl}amino)-3-(6-phenylpyridin-3-yl)propanoic acid, (2R)-2-({[(9H-fluoren-9-yl)methoxy]carbonyl}amino)-3-(6-phenylpyridin-3-yl)propanoic acid, (2S)-3-(2-carbamoylpyridin-4-yl)-2-({[(9H-fluoren-9-yl)methoxy]carbonyl}amino)propanoic acid, (2S)-3-(3-carbamoylpyridin-4-yl)-2-({[(9H-fluoren-9-yl)methoxy]carbonyl}amino)propanoic acid, (2S)-2-({[(9H-fluoren-9-yl)methoxy]carbonyl}amino)-3-(1H-indazol-5-yl)propanoic acid, (2R)-2-({[(9H-fluoren-9-yl)methoxy]carbonyl}amino)-3-(1H-indazol-5-yl)propanoic acid, (2S)-3-(1-acetylpiperidin-4-yl)-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-{1-[(prop-2-en-1-yloxy)carbonyl]piperidin-4-yl}propanoic acid, (2S)-3-{1-[(tert-butoxy)carbonyl]piperidin-4-yl}-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-{1-[1-(triphenylmethyl)-1H-pyrazole-4-carbonyl]piperidin-4-yl}propanoic acid, (2S)-2-({[(9H-fluoren-9-yl)methoxy]carbonyl}(methyl)amino)-3-{1-[3-(triphenylmethoxy)propanoyl]piperidin-4-yl}propanoic acid, (2S)-3-{1-[2-(tert-butoxy)acetyl]piperidin-4-yl}-2-({[(9H-fluoren-9-yl)methoxy]carbonyl}(methyl)amino)propanoic acid, (2S)-2-({[(9H-fluoren-9-yl)methoxy]carbonyl}amino)-7-methoxyheptanoic acid, (2S)-3-[3′-(tert-butoxy)-[1,1′-biphenyl]-4-yl]-2-({[(9H-fluoren-9-yl)methoxy]carbonyl}amino)propanoic acid, (2S)-2-({[(9H-fluoren-9-yl)methoxy]carbonyl}amino)-4-methanesulfonamidobutanoic acid, (2R)-2-({[(9H-fluoren-9-yl)methoxy]carbonyl}(methyl)amino)-3-{[(prop-2-en-1-yloxy)carbonyl]amino}propanoic acid, (2S)-3-{[(tert-butoxy)carbonyl]amino}-2-({[(9H-fluoren-9-yl)methoxy]carbonyl}(methyl)amino)propanoic acid, (2R)-2-({[(9H-fluoren-9-yl)methoxy]carbonyl}amino)-3-{3-[2-oxo-2-(prop-2-en-1-yloxy)ethyl]phenyl}propanoic acid, (2S)-2-({[(9H-fluoren-9-yl)methoxy]carbonyl}amino)-3-{3-[2-oxo-2-(prop-2-en-1-yloxy)ethyl]phenyl}propanoic acid, (2R)-3-{3-[2-(tert-butoxy)-2-oxoethyl]phenyl}-2-({[(9H-fluoren-9-yl)methoxy]carbonyl}amino)propanoic acid, (2S)-3-{3-[2-(tert-butoxy)-2-oxoethyl]phenyl}-2-({[(9H-fluoren-9-yl)methoxy]carbonyl}amino)propanoic acid, (2R)-2-({[(9H-fluoren-9-yl)methoxy]carbonyl}amino)-3-{3-[2-oxo-2-(prop-2-en-1-yloxy)ethoxy]phenyl}propanoic acid, (2S)-2-({[(9H-fluoren-9-yl)methoxy]carbonyl}amino)-3-{3-[2-oxo-2-(prop-2-en-1-yloxy)ethoxy]phenyl}propanoic acid, (2R)-3-{3-[2-(tert-butoxy)-2-oxoethoxy]phenyl}-2-({[(9H-fluoren-9-yl)methoxy]carbonyl}amino)propanoic acid, (2S)-3-{3-[2-(tert-butoxy)-2-oxoethoxy]phenyl}-2-({[(9H-fluoren-9-yl)methoxy]carbonyl}amino)propanoic acid, (2S)-3-[3-(acetamidomethyl)phenyl]-2-({[(9H-fluoren-9-yl)methoxy]carbonyl}(methyl)amino)propanoic acid, (2S)-3-(3-carbamoylphenyl)-2-({[(9H-fluoren-9-yl)methoxy]carbonyl}(methyl)amino)propanoic acid, (2R)-3-(3-carbamoylphenyl)-2-({[(9H-fluoren-9-yl)methoxy]carbonyl}amino)propanoic acid, (2S)-3-[3-(benzylcarbamoyl)phenyl]-2-({[(9H-fluoren-9-yl)methoxy]carbonyl}amino)propanoic acid, (2S)-3-[3-(dimethylcarbamoyl)phenyl]-2-({[(9H-fluoren-9-yl)methoxy]carbonyl}amino)propanoic acid, (2S)-2-({[(9H-fluoren-9-yl)methoxy]carbonyl}(methyl)amino)-3-(3-{[(prop-2-en-1-yloxy)carbonyl]amino}phenyl)propanoic acid, (2S)-3-(4-carbamoylphenyl)-2-({[(9H-fluoren-9-yl)methoxy]carbonyl}(methyl)amino)propanoic acid, (2S)-3-[4-(dimethylcarbamoyl)phenyl]-2-({[(9H-fluoren-9-yl)methoxy]carbonyl}amino)propanoic acid, (2S)-2-({[(9H-fluoren-9-yl)methoxy]carbonyl}amino)-3-{4-[(2,5,8,11-tetraoxatridecan-13-yl)carbamoyl]phenyl}propanoic acid, (2S)-4-(2-{[(tert-butoxy)carbonyl]amino}pyridin-3-yl)-2-({[(9H-fluoren-9-yl)methoxy]carbonyl}amino)butanoic acid, (2S)-6-(carbamoylamino)-2-({[(9H-fluoren-9-yl)methoxy]carbonyl}(methyl)amino)hexanoic acid, (2S)-4-{4-[(tert-butoxy)carbonyl]phenyl}-2-({[(9H-fluoren-9-yl)methoxy]carbonyl}amino)butanoic acid, (2S)-2-({[(9H-fluoren-9-yl)methoxy]carbonyl}(methyl)amino)-6-[(2H-1,2,3,4-tetrazol-5-yl)amino]hexanoic acid, (2S)-6-{[(tert-butoxy)carbonyl](1H-1,2,3,4-tetrazol-5-yl)amino}-2-({[(9H-fluoren-9-yl)methoxy]carbonyl}(methyl)amino)hexanoic acid, (2S)-6-(2-acetamidoacetamido)-2-({[(9H-fluoren-9-yl)methoxy]carbonyl}(methyl)amino)hexanoic acid, (2S)-6-[4-(tert-butoxy)-4-oxobutanamido]-2-({[(9H-fluoren-9-yl)methoxy]carbonyl}(methyl)amino)hexanoic acid, (2S)-6-(cyclopropylformamido)-2-({[(9H-fluoren-9-yl)methoxy]carbonyl}(methyl)amino)hexanoic acid, (2S)-2-({[(9H-fluoren-9-yl)methoxy]carbonyl}(methyl)amino)-6-propanamidohexanoic acid, (2S)-2-({[(9H-fluoren-9-yl)methoxy]carbonyl}(methyl)amino)-6-[3-(triphenylmethoxy)propanamido]hexanoic acid, (2S)-2-({[(9H-fluoren-9-yl)methoxy]carbonyl}(methyl)amino)-6-(2-methylpropanamido)hexanoic acid, (2S)-2-({[(9H-fluoren-9-yl)methoxy]carbonyl}(methyl)amino)-6-(3-methoxypropanamido)hexanoic acid, (2S)-2-({[(9H-fluoren-9-yl)methoxy]carbonyl}(methyl)amino)-6-[(2S)-2-(triphenylmethoxy)propanamido]hexanoic acid, (2S)-2-({[(9H-fluoren-9-yl)methoxy]carbonyl}(methyl)amino)-6-[(2R)-2-(triphenylmethoxy)propanamido]hexanoic acid, (2S)-2-({[(9H-fluoren-9-yl)methoxy]carbonyl}(methyl)amino)-6-(phenylformamido)hexanoic acid, (2S)-6-[2-(tert-butoxy)acetamido]-2-({[(9H-fluoren-9-yl)methoxy]carbonyl}(methyl)amino)hexanoic acid, (2S)-2-({[(9H-fluoren-9-yl)methoxy]carbonyl}(methyl)amino)-6-(2-methoxyacetamido)hexanoic acid, (2S)-2-({[(9H-fluoren-9-yl)methoxy]carbonyl}(methyl)amino)-6-(morpholin-4-yl)hexanoic acid, (2S)-2-({[(9H-fluoren-9-yl)methoxy]carbonyl}(methyl)amino)-6-methanesulfonamidohexanoic acid, (2S)-2-({[(9H-fluoren-9-yl)methoxy]carbonyl}(methyl)amino)-6-(2-oxoimidazolidin-1-yl)hexanoic acid, (2S)-2-({[(9H-fluoren-9-yl)methoxy]carbonyl}(methyl)amino)-6-(2-oxopyrrolidin-1-yl)hexanoic acid, (2S)-2-({[(9H-fluoren-9-yl)methoxy]carbonyl}(methyl)amino)-6-(2-oxo-1,3-diazinan-1-yl)hexanoic acid, (2S)-3-{4′-cyano-[1,1′-biphenyl]-3-yl}-2-({[(9H-fluoren-9-yl)methoxy]carbonyl}amino)propanoic acid, (2S)-2-({[(9H-fluoren-9-yl)methoxy]carbonyl}(methyl)amino)-6-(1H-1,2,3-triazol-1-yl)hexanoic acid, (2S)-5-acetamido-2-({[(9H-fluoren-9-yl)methoxy]carbonyl}(methyl)amino)pentanoic acid, (2S)-2-({[(9H-fluoren-9-yl)methoxy]carbonyl}amino)-5-methanesulfonamidopentanoic acid, (2S)-2-({[(9H-fluoren-9-yl)methoxy]carbonyl}amino)-4-{[(pyridin-4-yl)methyl]carbamoyl}butanoic acid hydrochloride, (2S)-2-({[(9H-fluoren-9-yl)methoxy]carbonyl}amino)-4-{[(pyridin-4-yl)methyl]carbamoyl}butanoic acid, (2S)-2-({[(9H-fluoren-9-yl)methoxy]carbonyl}amino)-4-{[(1s,3s)-3-{[(tert-butoxy)carbonyl]amino}cyclobutyl]carbamoyl}butanoic acid, (2S)-4-[(carbamoylmethyl)carbamoyl]-2-({[(9H-fluoren-9-yl)methoxy]carbonyl}(methyl)amino)butanoic acid, (2S)-2-({[(9H-fluoren-9-yl)methoxy]carbonyl}amino)-4-{[(pyrimidin-5-yl)methyl]carbamoyl}butanoic acid, (2S)-4-{[(3S)-1-[(tert-butoxy)carbonyl]pyrrolidin-3-yl]carbamoyl}-2-({[(9H-fluoren-9-yl)methoxy]carbonyl}amino)butanoic acid, (2S)-2-({[(9H-fluoren-9-yl)methoxy]carbonyl}amino)-3-(5,6,7,8-tetrahydronaphthalen-2-yl)propanoic acid, (2S)-3-{5-[2-(tert-butoxy)-2-oxoethoxy]-1-[(tert-butoxy)carbonyl]-1H-indol-3-yl}-2-({[(9H-fluoren-9-yl)methoxy]carbonyl}amino)propanoic acid, (2S)-2-({[(9H-fluoren-9-yl)methoxy]carbonyl}amino)-3-{3′-methanesulfonyl-[1,1′-biphenyl]-4-yl}propanoic acid, (2S)-2-({[(9H-fluoren-9-yl)methoxy]carbonyl}amino)-3-methanesulfonamidopropanoic acid, (2S)-3-(2-acetamidoacetamido)-2-({[(9H-fluoren-9-yl)methoxy]carbonyl}(methyl)amino)propanoic acid, (2S)-2-({[(9H-fluoren-9-yl)methoxy]carbonyl}amino)-3-[3-({[(prop-2-en-1-yloxy)carbonyl]amino}methyl)phenyl]propanoic acid, (2S)-2-({[(9H-fluoren-9-yl)methoxy]carbonyl}(methyl)amino)-3-(4-{[(prop-2-en-1-yloxy)carbonyl]amino}phenyl)propanoic acid, (2S)-2-({[(9H-fluoren-9-yl)methoxy]carbonyl}amino)-3-(1,2-oxazol-4-yl)propanoic acid, (2S)-5-(tert-butoxy)-2-({[(9H-fluoren-9-yl)methoxy]carbonyl}(methyl)amino)-5-oxopentanoic acid, (2S)-2-({[(9H-fluoren-9-yl)methoxy]carbonyl}(methyl)amino)-4-[(3-methylpentan-3-yl)oxy]-4-oxobutanoic acid, (2S)-2-({[(9H-fluoren-9-yl)methoxy]carbonyl}(methyl)amino)-3-(triphenylmethoxy)propanoic acid, (2S)-3-(3-chlorophenyl)-2-({[(9H-fluoren-9-yl)methoxy]carbonyl}amino)propanoic acid, (2S)-2-({[(9H-fluoren-9-yl)methoxy]carbonyl}amino)-4-phenylbutanoic acid, (2S)-2-({[(9H-fluoren-9-yl)methoxy]carbonyl}amino)-4-(4-fluorophenyl)butanoic acid, (2S)-4-(4-{[(tert-butoxy)carbonyl]amino}phenyl)-2-({[(9H-fluoren-9-yl)methoxy]carbonyl}amino)butanoic acid, (2S)-2-({[(9H-fluoren-9-yl)methoxy]carbonyl}(methyl)amino)-3-(6-oxo-1,6-dihydropyridin-3-yl)propanoic acid, (2S)-2-({[(9H-fluoren-9-yl)methoxy]carbonyl}(methyl)amino)-3-(4-methoxyphenyl)propanoic acid, (2S)-2-({[(9H-fluoren-9-yl)methoxy]carbonyl}amino)-3-(6-oxo-1,6-dihydropyridin-3-yl)propanoic acid, (2S)-3-(5,6-dimethoxypyridin-3-yl)-2-({[(9H-fluoren-9-yl)methoxy]carbonyl}amino)propanoic acid, (2S)-2-({[(9H-fluoren-9-yl)methoxy]carbonyl}amino)-3-[2-(methylcarbamoyl)pyridin-4-yl]propanoic acid, (2S)-2-({[(9H-fluoren-9-yl)methoxy]carbonyl}amino)-3-(4-methanesulfonylphenyl)propanoic acid, (2S)-2-({[(9H-fluoren-9-yl)methoxy]carbonyl}(methyl)amino)-3-{4-[(prop-2-en-1-yloxy)carbonyl]phenyl}propanoic acid, (2S)-3-(4-acetamidophenyl)-2-({[(9H-fluoren-9-yl)methoxy]carbonyl}amino)propanoic acid, (2S)-2-({[(9H-fluoren-9-yl)methoxy]carbonyl}amino)-3-(6-methoxypyridin-3-yl)propanoic acid, (2S)-3-{4-[(tert-butoxy)carbonyl]phenyl}-2-({[(9H-fluoren-9-yl)methoxy]carbonyl}(methyl)amino)propanoic acid, (2S)-3-{3-[(tert-butoxy)carbonyl]phenyl}-2-({[(9H-fluoren-9-yl)methoxy]carbonyl}(methyl)amino)propanoic acid, (2S)-3-{5-[(tert-butoxy)carbonyl]pyridin-3-yl}-2-({[(9H-fluoren-9-yl)methoxy]carbonyl}amino)propanoic acid, (2S)-4-{3-[(tert-butoxy)carbonyl]phenyl}-2-({[(9H-fluoren-9-yl)methoxy]carbonyl}amino)butanoic acid, (2S)-2-({[(9H-fluoren-9-yl)methoxy]carbonyl}amino)-3-(2-methoxypyridin-4-yl)propanoic acid, (2S)-3-{3-[(tert-butoxy)carbonyl]-4-hydroxyphenyl}-2-({[(9H-fluoren-9-yl)methoxy]carbonyl}amino)propanoic acid, (2S)-3-[4-(tert-butoxy)-3-methylphenyl]-2-({[(9H-fluoren-9-yl)methoxy]carbonyl}amino)propanoic acid, (2S)-3-(2-{[(tert-butoxy)carbonyl]amino}pyridin-4-yl)-2-({[(9H-fluoren-9-yl)methoxy]carbonyl}(methyl)amino)propanoic acid, (2S)-3-[4-({[(tert-butoxy)carbonyl]amino}methyl)phenyl]-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-[4-({[(prop-2-en-1-yloxy)carbonyl]amino}methyl)phenyl]propanoic acid, (2S)-3-{3-[(3-{[(tert-butoxy)carbonyl]amino}propyl)carbamoyl]phenyl}-2-({[(9H-fluoren-9-yl)methoxy]carbonyl}(methyl)amino)propanoic acid, (2S)-3-{3-[(2-{1[(tert-butoxy)carbonyl]amino}ethyl)carbamoyl]phenyl}-2-({[(9H-fluoren-9-yl)methoxy]carbonyl}(methyl)amino)propanoic acid, (2S)-3-(6-{[(tert-butoxy)carbonyl](methyl)amino}pyridin-3-yl)-2-({[(9H-fluoren-9-yl)methoxy]carbonyl}amino)propanoic acid, (2S)-3-[3-({[(tert-butoxy)carbonyl]amino}methyl)phenyl]-2-({[(9H-fluoren-9-yl)methoxy]carbonyl}(methyl)amino)propanoic acid, (2S,4R)-4-(tert-butoxy)-1-{[(9H-fluoren-9-yl)methoxy]carbonyl}pyrrolidine-2-carboxylic acid, (2R,4S)-4-{[(tert-butoxy)carbonyl]amino}-1-{[(9H-fluoren-9-yl)methoxy]carbonyl}pyrrolidine-2-carboxylic acid, (2R,4R)-1-{[(9H-fluoren-9-yl)methoxy]carbonyl}-4-hydroxypyrrolidine-2-carboxylic acid, (2S)-2-({[(9H-fluoren-9-yl)methoxy]carbonyl}(methyl)amino)pentanoic acid, (2S)-3-cyclobutyl-2-({[(9H-fluoren-9-yl)methoxy]carbonyl}amino)propanoic acid, (2S)-3-cyclopentyl-2-({[(9H-fluoren-9-yl)methoxy]carbonyl}amino)propanoic acid, (2S)-4-ethyl-2-({[(9H-fluoren-9-yl)methoxy]carbonyl}amino)hexanoic acid, (2S)-2-({[(9H-fluoren-9-yl)methoxy]carbonyl}amino)-7,7,7-trifluoroheptanoic acid, (2S)-4-cyclobutyl-2-({[(9H-fluoren-9-yl)methoxy]carbonyl}amino)butanoic acid, (2S)-4-cyclopentyl-2-({[(9H-fluoren-9-yl)methoxy]carbonyl}amino)butanoic acid, (2S)-2-({[(9H-fluoren-9-yl)methoxy]carbonyl}amino)-3-(1-methyl-1H-pyrazol-4-yl)propanoic acid, (2S)-2-({[(9H-fluoren-9-yl)methoxy]carbonyl}amino)-3-(1,3-oxazol-5-yl)propanoic acid, (2S)-2-({[(9H-fluoren-9-yl)methoxy]carbonyl}amino)-3-(1,3-oxazol-4-yl)propanoic acid, (2S)-2-({[(9H-fluoren-9-yl)methoxy]carbonyl}amino)-3-[1-(triphenylmethyl)-1H-pyrazol-4-yl]propanoic acid, (2S)-2-({[(9H-fluoren-9-yl)methoxy]carbonyl}amino)-3-(5-hydroxy-1H-indol-3-yl)propanoic acid, (2S)-2-({[(9H-fluoren-9-yl)methoxy]carbonyl}amino)-3-(naphthalen-1-yl)propanoic acid, (2S)-2-({[(9H-fluoren-9-yl)methoxy]carbonyl}(methyl)amino)-3-(naphthalen-2-yl)propanoic acid, (2S)-3-{1-[(tert-butoxy)carbonyl]-7-methoxy-1H-indol-3-yl}-2-({[(9H-fluoren-9-yl)methoxy]carbonyl}amino)propanoic acid, (2S)-2-({[(9H-fluoren-9-yl)methoxy]carbonyl}amino)-3-(quinolin-6-yl)propanoic acid, (2S)-3-{1-[(tert-butoxy)carbonyl]-5-hydroxy-1H-indol-3-yl}-2-({[(9H-fluoren-9-yl)methoxy]carbonyl}amino)propanoic acid, (2S)-2-({[(9H-fluoren-9-yl)methoxy]carbonyl}amino)-3-{1H-pyrrolo[3,2-c]pyridin-3-yl}propanoic acid, (2S)-2-({[(9H-fluoren-9-yl)methoxy]carbonyl}amino)-3-{1-methyl-1H-pyrrolo[2,3-b]pyridin-3-yl}propanoic acid, (2S)-3-(1-{2-[(4-{[1-(4,4-dimethyl-2,6-dioxocyclohexylidene)-3-methylbutyl]amino}phenyl)methoxy]-2-oxoethyl}-1H-indol-3-yl)-2-({[(9H-fluoren-9-yl)methoxy]carbonyl}amino)propanoic acid, (2S)-2-({[(9H-fluoren-9-yl)methoxy]carbonyl}(methyl)amino)-3-(1H-indazol-5-yl)propanoic acid, (2S)-3-[5-(benzyloxy)-1H-indol-3-yl]-2-({[(9H-fluoren-9-yl)methoxy]carbonyl}amino)propanoic acid, (2S)-3-{1-[(tert-butoxy)carbonyl]-5-[(4-methoxy phenyl)methoxy]-1H-indol-3-yl}-2-({[(9H-fluoren-9-yl)methoxy]carbonyl}amino)propanoic acid, (2S,3S)-3-(tert-butoxy)-2-({[(9H-fluoren-9-yl)methoxy]carbonyl}amino)butanoic acid, (2S)-2-({[(9H-fluoren-9-yl)methoxy]carbonyl}amino)-3-(oxan-4-yl)propanoic acid (2S)-2-({[(9H-fluoren-9-yl)methoxy]carbonyl}amino)-3-[(1r,4r)-4-methoxycyclohexyl]propanoic acid, (2S)-2-({[(9H-fluoren-9-yl)methoxy]carbonyl}amino)-3-[(1r,4r)-4-hydroxycyclohexyl]propanoic acid, (2S)-3-butoxy-2-({[(9H-fluoren-9-yl)methoxy]carbonyl}amino)propanoic acid, (2S)-2-({[(9H-fluoren-9-yl)methoxy]carbonyl}amino)-3-(pentyloxy)propanoic acid, (2S)-2-({[(9H-fluoren-9-yl)methoxy]carbonyl}amino)-4-propoxybutanoic acid, (2S)-4-ethoxy-2-({[(9H-fluoren-9-yl)methoxy]carbonyl}amino)butanoic acid, (2S)-4-butoxy-2-({[(9H-fluoren-9-yl)methoxy]carbonyl}amino)butanoic acid, (2S)-3-(cyclopentyloxy)-2-({[(9H-fluoren-9-yl)methoxy]carbonyl}amino)propanoic acid, (2S)-3-(cyclobutylmethoxy)-2-({[(9H-fluoren-9-yl)methoxy]carbonyl}amino)propanoic acid, (2S)-4-(cyclopentyloxy)-2-({[(9H-fluoren-9-yl)methoxy]carbonyl}amino)butanoic acid, (2S)-2-({[(9H-fluoren-9-yl)methoxy]carbonyl}amino)-4-(propan-2-yloxy)butanoic acid, (2S)-2-({[(9H-fluoren-9-yl)methoxy]carbonyl}amino)-3-[methyl(phenyl)amino]propanoic acid hydrochloride, (2S)-4-acetamido-2-({[(9H-fluoren-9-yl)methoxy]carbonyl}amino)butanoic acid, (2R)-3-{1-[(tert-butoxy)carbonyl]piperidin-4-yl}-2-({[(9H-fluoren-9-yl)methoxy]carbonyl}amino)propanoic acid, (2R)-4-{[(tert-butoxy)carbonyl]amino}-2-({[(9H-fluoren-9-yl)methoxy]carbonyl}amino)butanoic acid, (2R)-7-{[(tert-butoxy)carbonyl]amino}-2-({[(9H-fluoren-9-yl)methoxy]carbonyl}amino)heptanoic acid, (2S)-2-({[(9H-fluoren-9-yl)methoxy]carbonyl}amino)-7-{[(prop-2-en-1-yloxy)carbonyl]amino}heptanoic acid, (2R)-2-({[(9H-fluoren-9-yl)methoxy]carbonyl}amino)-7-{[(prop-2-en-1-yloxy)carbonyl]amino}heptanoic acid, (2S)-2-({[(9H-fluoren-9-yl)methoxy]carbonyl}amino)-3-(1-methanesulfonylpiperidin-4-yl)propanoic acid, (2R)-5-(carbamoylamino)-2-({[(9H-fluoren-9-yl)methoxy]carbonyl}amino)pentanoic acid, (2S)-6-(carbamoylamino)-2-({[(9H-fluoren-9-yl)methoxy]carbonyl}amino)hexanoic acid, (2S)-2-({[(9H-fluoren-9-yl)methoxy]carbonyl}(methyl)amino)-6-oxo-6-(prop-2-en-1-yloxy)hexanoic acid, (2S)-6-(tert-butoxy)-2-({[(9H-fluoren-9-yl)methoxy]carbonyl}(methyl)amino)-6-oxohexanoic acid, (2S)-5-(dimethylcarbamoyl)-2-({[(9H-fluoren-9-yl)methoxy]carbonyl}(methyl)amino)pentanoic acid, (2R)-2-({[(9H-fluoren-9-yl)methoxy]carbonyl}(methyl)amino)-4-[(3-methylpentan-3-yl)oxy]-4-oxobutanoic acid, (2S)-3-(3-carbamoylphenyl)-2-({[(9H-fluoren-9-yl)methoxy]carbonyl}amino)propanoic acid, (2S)-3-(4-carbamoylphenyl)-2-({[(9H-fluoren-9-yl)methoxy]carbonyl}amino)propanoic acid, (2R)-3-(4-carbamoylphenyl)-2-({[(9H-fluoren-9-yl)methoxy]carbonyl}(methyl)amino)propanoic acid, (2R)-3-(3-carbamoylphenyl)-2-({[(9H-fluoren-9-yl)methoxy]carbonyl}(methyl)amino)propanoic acid, (2S)-3-(4-{[(tert-butoxy)carbonyl]amino}phenyl)-2-({[(9H-fluoren-9-yl)methoxy]carbonyl}(methyl)amino)propanoic acid, (2R)-2-({[(9H-fluoren-9-yl)methoxy]carbonyl}amino)-3-[3-({[(prop-2-en-1-yloxy)carbonyl]amino}methyl)phenyl]propanoic acid, (2S)-3-[4-(acetamidomethyl)phenyl]-2-({[(9H-fluoren-9-yl)methoxy]carbonyl}(methyl)amino)propanoic acid, 2-({[(9H-fluoren-9-yl)methoxy]carbonyl}[2-(N-methylmethanesulfonamido)ethyl]amino)acetic acid, (2S)-4-(carbamoylamino)-2-({[(9H-fluoren-9-yl)methoxy]carbonyl}amino)butanoic acid, (2S)-5-(cyclohexylformamido)-2-({[(9H-fluoren-9-yl)methoxy]carbonyl}amino)pentanoic acid, (2S)-3-{3-[(3-{[(tert-butoxy)carbonyl]amino}propyl)carbamoyl]phenyl}-2-({[(9H-fluoren-9-yl)methoxy]carbonyl}amino)propanoic acid, (2S)-3-(7-chloro-1-methyl-1H-indol-3-yl)-2-({[(9H-fluoren-9-yl)methoxy]carbonyl}amino)propanoic acid, (2S)-2-({[(9H-fluoren-9-yl)methoxy]carbonyl}amino)-3-[4-(1-methyl-1H-pyrazol-4-yl)phenyl]propanoic acid, (2S)-2-({[(9H-fluoren-9-yl)methoxy]carbonyl}amino)-3-[4-(pyridin-3-yl)phenyl]propanoic acid hydrochloride, (2S)-2-({[(9H-fluoren-9-yl)methoxy]carbonyl}(methyl)amino)-3-(isoquinolin-7-yl)propanoic acid, (2S)-2-({[(9H-fluoren-9-yl)methoxy]carbonyl}amino)-3-(2-phenylpyridin-4-yl)propanoic acid, (2S)-2-({[(9H-fluoren-9-yl)methoxy]carbonyl}amino)-3-(quinolin-5-yl)propanoic acid, (2S)-3-{3-[(2-{[(tert-butoxy)carbonyl]amino}ethyl)carbamoyl]phenyl}-2-({[(9H-fluoren-9-yl)methoxy]carbonyl}amino)propanoic acid, 2-({[(9H-fluoren-9-yl)methoxy]carbonyl}({2-[(triphenylmethyl)carbamoyl]ethyl})amino)acetic acid, (2S)-2-({[(9H-fluoren-9-yl)methoxy]carbonyl}amino)-3-[4-(pyridin-2-yl)phenyl]propanoic acid hydrochloride, (2S)-4-[(2-carbamoylethyl)carbamoyl]-2-({[(9H-fluoren-9-yl)methoxy]carbonyl}amino)butanoic acid, (2S)-3-[4-(1-acetylpiperidin-4-yl)phenyl]-2-({[(9H-fluoren-9-yl)methoxy]carbonyl}amino)propanoic acid, (2S)-2-({[(9H-fluoren-9-yl)methoxy]carbonyl}(methyl)amino)-3-(quinolin-7-yl)propanoic acid, (2S)-2-({[(9H-fluoren-9-yl)methoxy]carbonyl}amino)-4-oxo-4-(pyrrolidin-1-yl)butanoic acid, (2S)-3-[1-(3-{[(tert-butoxy)carbonyl]amino}propyl)-1H-indol-3-yl]-2-({[(9H-fluoren-9-yl)methoxy]carbonyl}amino)propanoic acid, 2-[(2-{[1,1′-biphenyl]-4-yl}ethyl)({[(9H-fluoren-9-yl)methoxy]carbonyl})amino]acetic acid, (2S)-3-{1-[(tert-butoxy)carbonyl]-5,6-dichloro-1H-indol-3-yl}-2-({[(9H-fluoren-9-yl)methoxy]carbonyl}amino)propanoic acid, (2S)-3-(4-{1-[(tert-butoxy)carbonyl]piperidin-4-yl}phenyl)-2-({[(9H-fluoren-9-yl)methoxy]carbonyl}amino)propanoic acid, (2S)-2-({[(9H-fluoren-9-yl)methoxy]carbonyl}amino)-3-(4-{1-[(prop-2-en-1-yloxy)carbonyl]piperidin-4-yl}phenyl)propanoic acid, (2S)-3-(6-carbamoylpyridin-3-yl)-2-({[(9H-fluoren-9-yl)methoxy]carbonyl}amino)propanoic acid, (2S)-3-{1-[(tert-butoxy)carbonyl]-1H-pyrrolo[3,2-b]pyridin-3-yl}-2-({[(9H-fluoren-9-yl)methoxy]carbonyl}amino)propanoic acid, (2R)-2-({[(9H-fluoren-9-yl)methoxy]carbonyl}amino)-3-{3-[(prop-2-en-1-yloxy)carbonyl]phenyl}propanoic acid, (2R)-3-{4-[(tert-butoxy)carbonyl]phenyl}-2-({[(9H-fluoren-9-yl)methoxy]carbonyl}amino)propanoic acid, (2R)-2-({[(9H-fluoren-9-yl)methoxy]carbonyl}amino)-3-{4-[(prop-2-en-1-yloxy)carbonyl]phenyl}propanoic acid, (2S)-2-({[(9H-fluoren-9-yl)methoxy]carbonyl}amino)-3-[5-methyl-1-(triphenylmethyl)-1H-imidazol-4-yl]propanoic acid, (2R)-3-{[(2,4-dimethoxyphenyl)methyl]sulfamoyl}-2-({[(9H-fluoren-9-yl)methoxy]carbonyl}amino)propanoic acid, (S)-2-((((9H-fluoren-9-yl)methoxy)carbonyl)amino)-3-(5-phenylpyridin-3-yl)propanoic acid, (S)-2-((((9H-fluoren-9-yl)methoxy)carbonyl)(methyl)amino)-3-(3,4-dichlorophenyl)propanoic acid, (S)-2-((((9H-fluoren-9-yl)methoxy)carbonyl)amino)-3-(4-(2-amino-2-oxoethyl)phenyl)propanoic acid, (S)-2-((((9H-fluoren-9-yl)methoxy)carbonyl)amino)-3-(2-carbamoylpyridin-3-yl)propanoic acid, (S)-2-((((9H-fluoren-9-yl)methoxy)carbonyl)amino)-3-(5,6-dimethylpyridin-3-yl)propanoic acid, (S)-2-((((9H-fluoren-9-yl)methoxy)carbonyl)amino)-3-(2,6-dimethylpyridin-4-yl)propanoic acid, (R)-2-((((9H-fluoren-9-yl)methoxy)carbonyl)amino)-3-(2-((tert-butoxycarbonyl)amino)pyridin-4-yl)propanoic acid, (S)-2-((((9H-fluoren-9-yl)methoxy)carbonyl)amino)-4-(5-(trifluoromethyl)pyridin-3-yl)butanoic acid, (S)-2-((((9H-fluoren-9-yl)methoxy)carbonyl)amino)-4-(2-(trifluoromethyl)pyridin-4-yl)butanoic acid, (R)-2-((((9H-fluoren-9-yl)methoxy)carbonyl)(methyl)amino)-3-cyclobutylpropanoic acid, (S)-2-((((9H-fluoren-9-yl)methoxy)carbonyl)(methyl)amino)-3-(2,3-dichlorophenyl)propanoic acid, (S)-2-((((9H-fluoren-9-yl)methoxy)carbonyl)amino)-3-(1-trityl-1,4,5,6-tetrahydrocyclopenta[c]pyrazol-3-yl)propanoic acid, (S)-2-((((9H-fluoren-9-yl)methoxy)carbonyl)amino)-3-(4-(tetrahydro-2H-pyran-4-yl)phenyl)propanoic acid, (S)-2-((((9H-fluoren-9-yl)methoxy)carbonyl)amino)-4-(3-(pentafluoro-16-sulfaneyl)phenyl)butanoic acid, (2S,4R)-1-(((9H-fluoren-9-yl)methoxy)carbonyl)-4-(2-chlorophenoxy)pyrrolidine-2-carboxylic acid, (2S,4R)-1-(((9H-fluoren-9-yl)methoxy)carbonyl)-4-(2-fluorophenoxy)pyrrolidine-2-carboxylic acid, (2S,4R)-1-(((9H-fluoren-9-yl)methoxy)carbonyl)-4-(3-chlorophenoxy)pyrrolidine-2-carboxylic acid, (2S,4R)-1-(((9H-fluoren-9-yl)methoxy)carbonyl)-4-(3-fluorophenoxy)pyrrolidine-2-carboxylic acid, (2S,4R)-1-(((9H-fluoren-9-yl)methoxy)carbonyl)-4-(4-chlorophenoxy)pyrrolidine-2-carboxylic acid, (2S,4R)-1-(((9H-fluoren-9-yl)methoxy)carbonyl)-4-(4-fluorophenoxy)pyrrolidine-2-carboxylic acid, (2S)-2-({[(9H-fluoren-9-yl)methoxy]carbonyl}amino)-3-{2-fluoro-[1,1′-biphenyl]-4-yl}propanoic acid, (S)-2-((((9H-fluoren-9-yl)methoxy)carbonyl)amino)-3-(6-(2-((tert-butoxycarbonyl)amino)ethoxy)pyridin-3-yl)propanoic acid, (S)-2-((((9H-fluoren-9-yl)methoxy)carbonyl)amino)-4-(dimethylamino)butanoic acid hydrochloride, (S)-2-((((9H-fluoren-9-yl)methoxy)carbonyl)amino)-3-(4-(quinolin-4-yl)phenyl)propanoic acid, (S,Z)-2-((((9H-fluoren-9-yl)methoxy)carbonyl)amino)-3-(4-(2-methyl-3-((2,2,4,6,7-pentamethyl-2,3-dihydrobenzofuran-5-yl)sulfonyl)guanidino)phenyl)propanoic acid, (S)-2-((((9H-fluoren-9-yl)methoxy)carbonyl)amino)-3-(4-((1-((2,2,4,6,7-pentamethyl-2,3-dihydrobenzofuran-5-yl)sulfonyl)-4,5-dihydro-1H-imidazol-2-yl)amino)phenyl)propanoic acid, (S)-2-((((9H-fluoren-9-yl)methoxy)carbonyl)amino)-3-(2-(dimethylcarbamoyl)-1-((2-(trimethylsilyl)ethoxy)methyl)-1H-imidazol-4-yl)propanoic acid, (S)-2-((((9H-fluoren-9-yl)methoxy)carbonyl)amino)-3-(2-(methylcarbamoyl)-1-((2-(trimethylsilyl)ethoxy)methyl)-1H-imidazol-4-yl)propanoic acid, (2S)-3-[({1-[(tert-butoxy)carbonyl]azetidin-3-yl}methyl)carbamoyl]-2-({[(9H-fluoren-9-yl)methoxy]carbonyl}amino)propanoic acid, (S)-2-((((9H-fluoren-9-yl)methoxy)carbonyl)amino)-3-(isoquinolin-8-yl)propanoic acid, (2S,4R)-1-(((9H-fluoren-9-yl)methoxy)carbonyl)-4-(pyridin-2-yloxy)pyrrolidine-2-carboxylic acid, (2S,4R)-1-(((9H-fluoren-9-yl)methoxy)carbonyl)-4-(pyridin-3-yloxy)pyrrolidine-2-carboxylic acid, (S)-2-((((9H-fluoren-9-yl)methoxy)carbonyl)(methyl)amino)-3-(thiazol-2-yl)propanoic acid, (S)-2-((((9H-fluoren-9-yl)methoxy)carbonyl)(methyl)amino)-3-(5-chloropyridin-3-yl)propanoic acid, (S)-2-((((9H-fluoren-9-yl)methoxy)carbonyl)(methyl)amino)-3-(5-(trifluoromethyl)pyridin-3-yl)propanoic acid, (2S)-3-(2,6-dichloropyridin-4-yl)-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-(oxan-4-yl)propanoic acid, (2R)-2-({[(9H-fluoren-9-yl)methoxy]carbonyl}(methyl)amino)-3-{1-[(prop-2-en-1-yloxy)carbonyl]piperidin-4-yl}propanoic acid, (2R)-2-({[(9H-fluoren-9-yl)methoxy]carbonyl}(methyl)amino)-3-(oxan-4-yl)propanoic acid, (2R)-6-amino-2-({[(9H-fluoren-9-yl)methoxy]carbonyl}(methyl)amino)hexanoic acid hydrochloride, (2R)-2-({[(9H-fluoren-9-yl)methoxy]carbonyl}(methyl)amino)-6-{[(prop-2-en-1-yloxy)carbonyl]amino}hexanoic acid, (2S)-3-(2,3-dimethylphenyl)-2-({[(9H-fluoren-9-yl)methoxy]carbonyl}(methyl)amino)propanoic acid, (2S)-3-[3,5-bis(trifluoromethyl)phenyl]-2-({[(9H-fluoren-9-yl)methoxy]carbonyl}(methyl)amino)propanoic acid, (2S)-3-[3-chloro-5-(trifluoromethyl)phenyl]-2-({[(9H-fluoren-9-yl)methoxy]carbonyl}(methyl)amino)propanoic acid, (2S)-3-(3-ethylphenyl)-2-({[(9H-fluoren-9-yl)methoxy]carbonyl}(methyl)amino)propanoic acid, (2S)-2-({[(9H-fluoren-9-yl)methoxy]carbonyl}(methyl)amino)-6,6,6-trifluorohexanoic acid, (2S)-5-{[(tert-butoxy)carbonyl](methyl)amino}-2-({[(9H-fluoren-9-yl)methoxy]carbonyl}(methyl)amino)pentanoic acid, (2S)-2-({[(9H-fluoren-9-yl)methoxy]carbonyl}(methyl)amino)-5-{methyl[(prop-2-en-1-yloxy)carbonyl]amino}pentanoic acid, (2S)-4-(cyclohexyloxy)-2-({[(9H-fluoren-9-yl)methoxy]carbonyl}amino)butanoic acid, (2S)-2-({[(9H-fluoren-9-yl)methoxy]carbonyl}amino)-4-(oxan-4-yloxy)butanoic acid, (2S)-3-(2,2-dimethylpropoxy)-2-({[(9H-fluoren-9-yl)methoxy]carbonyl}amino)propanoic acid, (2S)-2-({[(9H-fluoren-9-yl)methoxy]carbonyl}(methyl)amino)-3-[2-oxo-2-(prop-2-en-1-yloxy)ethoxy]propanoic acid, (2R)-3-(3,4-dichlorophenyl)-2-({[(9H-fluoren-9-yl)methoxy]carbonyl}(methyl)amino)propanoic acid, (2R)-2-({[(9H-fluoren-9-yl)methoxy]carbonyl}amino)-3-(6-oxo-1,6-dihydropyridin-3-yl)propanoic acid, (2R)-4-(4-{[(tert-butoxy)carbonyl]amino}phenyl)-2-({[(9H-fluoren-9-yl)methoxy]carbonyl}amino)butanoic acid, (2R)-2-({[(9H-fluoren-9-yl)methoxy]carbonyl}(methyl)amino)-3-(6-methoxypyridin-3-yl)propanoic acid, (2S)-2-({[(9H-fluoren-9-yl)methoxy]carbonyl}(methyl)amino)-3-(2-methoxypyridin-4-yl)propanoic acid, (2R)-2-({[(9H-fluoren-9-yl)methoxy]carbonyl}(methyl)amino)-3-(6-oxo-1,6-dihydropyridin-3-yl)propanoic acid, (2S)-3-{3-[(tert-butoxy)carbonyl]-4-hydroxyphenyl}-2-({[(9H-fluoren-9-yl)methoxy]carbonyl}(methyl)amino)propanoic acid, (2S)-2-({[(9H-fluoren-9-yl)methoxy]carbonyl}(methyl)amino)-4-(4-fluorophenyl)butanoic acid, (2S)-2-({[(9H-fluoren-9-yl)methoxy]carbonyl}(methyl)amino)-3-(1,3-oxazol-4-yl)propanoic acid, (2S)-2-({[(9H-fluoren-9-yl)methoxy]carbonyl}(methyl)amino)-3-(1-methyl-1H-pyrazol-4-yl)propanoic acid, (2R)-2-({[(9H-fluoren-9-yl)methoxy]carbonyl}amino)-3-(5-phenylpyridin-3-yl)propanoic acid, (2R)-2-({[(9H-fluoren-9-yl)methoxy]carbonyl}amino)-3-(2-phenylpyridin-4-yl)propanoic acid, (2S)-2-({[(9H-fluoren-9-yl)methoxy]carbonyl}(methyl)amino)-3-(quinolin-6-yl)propanoic acid, (2R)-2-({[(9H-fluoren-9-yl)methoxy]carbonyl}amino)-3-(quinolin-5-yl)propanoic acid, (2S)-2-({[(9H-fluoren-9-yl)methoxy]carbonyl}amino)-3-(isoquinolin-7-yl)propanoic acid, (2S)-2-({[(9H-fluoren-9-yl)methoxy]carbonyl}(methyl)amino)-3-(isoquinolin-8-yl)propanoic acid, (2R,4R)-4-{[(tert-butoxy)carbonyl]amino}-1-{[(9H-fluoren-9-yl)methoxy]carbonyl}pyrrolidine-2-carboxylic acid, (2S)-4-ethoxy-2-({[(9H-fluoren-9-yl)methoxy]carbonyl}(methyl)amino)butanoic acid, (2S)-3-butoxy-2-({[(9H-fluoren-9-yl)methoxy]carbonyl}(methyl)amino)propanoic acid, (2S)-3-{[(2,4-dimethoxyphenyl)methyl]sulfamoyl}-2-({[(9H-fluoren-9-yl)methoxy]carbonyl}amino)propanoic acid, (2S)-2-({[(9H-fluoren-9-yl)methoxy]carbonyl}(methyl)amino)-4-propoxybutanoic acid, and (2S)-2-({[(9H-fluoren-9-yl)methoxy]carbonyl}amino)-3-[4-({[(prop-2-en-1-yloxy)carbonyl]amino}methyl)phenyl]propanoic acid.

5. An enantiomer, diastereomer or racemate of the amino acid derivative according to claim 1.

6. The amino acid derivative according to claim 2, wherein one or more functional groups within the amino acid are protected by protecting groups.

7. An amino acid derivative according to claim 6, wherein the amino acid is selected from the group consisting of (2S)-2-[[(9H-fluoren-9-ylmethoxy)carbonyl]amino]-6-(4-methylpiperazine-1-carbonylamino)hexanoic acid hydrochloride, (2S)-6-({4-[2-(tert-butoxy)-2-oxoethyl]piperazine-1-carbonyl}amino)-2-({[(9H-fluoren-9-yl)methoxy]carbonyl}amino)hexanoic acid, (2S)-2-({[(9H-fluoren-9-yl)methoxy]carbonyl}amino)-6-({methyl[(2S,3R,4R,5R)-2,3,4,5-tetrahydroxy-6-{[tris(propan-2-yl)silyl]oxy}hexyl]carbamoyl}amino)hexanoic acid”, (2R)-6-({[(3R)-1-[2-(tert-butoxy)-2-oxoethyl]pyrrolidin-3-yl]carbamoyl}amino)-2-({[(9H-fluoren-9-yl)methoxy]carbonyl}amino)hexanoic acid, (2S)-6-[(4-{2-[(tert-butyldimethylsilyl)oxy]ethyl}piperazine-1-carbonyl)amino]-2-({[(9H-fluoren-9-yl)methoxy]carbonyl}amino)hexanoic acid, (2R)-6-({4-[2-(tert-butoxy)-2-oxoethyl]piperazine-1-carbonyl}amino)-2-({[(9H-fluoren-9-yl)methoxy]carbonyl}amino)hexanoic acid, (2S)-6-({4-[2-(tert-butoxy)-2-oxoethyl]piperazine-1-carbonyl}amino)-2-({[(9H-fluoren-9-yl)methoxy]carbonyl}(methyl)amino)hexanoic acid, (2R)-6-({4-[2-(tert-butoxy)-2-oxoethyl]piperazine-1-carbonyl}amino)-2-({[(9H-fluoren-9-yl)methoxy]carbonyl}(methyl)amino)hexanoic acid, (2S)-2-({[(9H-fluoren-9-yl)methoxy]carbonyl}(methyl)amino)-6-({methyl[(2S,3R,4R,5R)-2,3,4,5-tetrahydroxy-6-{[tris(propan-2-yl)silyl]oxy}hexyl]carbamoyl}amino)hexanoic acid, (2R)-2-({[(9H-fluoren-9-yl)methoxy]carbonyl}amino)-6-({methyl[(2S,3R,4R,5R)-2,3,4,5-tetrahydroxy-6-{[tris(propan-2-yl)silyl]oxy}hexyl]carbamoyl}amino)hexanoic acid, (2R)-6-[(4-{2-[(tert-butyldimethylsilyl)oxy]ethyl}piperazine-1-carbonyl)amino]-2-({[(9H-fluoren-9-yl)methoxy]carbonyl}amino)hexanoic acid, (2S)-3-({4-[2-(tert-butoxy)-2-oxoethyl]piperazine-1-carbonyl}amino)-2-({[(9H-fluoren-9-yl)methoxy]carbonyl}amino)propanoic acid, (2S)-3-({4-[2-(tert-butoxy)-2-oxoethyl]piperazine-1-carbonyl}amino)-2-({[(9H-fluoren-9-yl)methoxy]carbonyl}(methyl)amino)propanoic acid, (2S,4R)-4-({4-[2-(tert-butoxy)-2-oxoethyl]piperazine-1-carbonyl}amino)-1-{[(9H-fluoren-9-yl)methoxy]carbonyl}pyrrolidine-2-carboxylic acid, (2S,4S)-4-({4-[2-(tert-butoxy)-2-oxoethyl]piperazine-1-carbonyl}amino)-1-{[(9H-fluoren-9-yl)methoxy]carbonyl}pyrrolidine-2-carboxylic acid, (2S)-2-({[(9H-fluoren-9-yl)methoxy]carbonyl}amino)-6-[(4-{2-oxo-2-[(2-phenylpropan-2-yl)oxy]ethyl}piperazine-1-carbonyl)amino]hexanoic acid, (2S)-7-({4-[2-(tert-butoxy)-2-oxoethyl]piperazine-1-carbonyl}amino)-2-({[(9H-fluoren-9-yl)methoxy]carbonyl}amino)heptanoic acid, (2S)-3-{4-[2-({4-[2-(tert-butoxy)-2-oxoethyl]piperazine-1-carbonyl}amino)ethoxy]phenyl}-2-({[(9H-fluoren-9-yl)methoxy]carbonyl}amino)propanoic acid, (2R)-6-[({1-[2-(tert-butoxy)-2-oxoethyl]piperidin-4-yl}carbamoyl)amino]-2-({[(9H-fluoren-9-yl)methoxy]carbonyl}amino)hexanoic acid, (2R)-6-{[({1-[2-(tert-butoxy)-2-oxoethyl]piperidin-4-yl}oxy)carbonyl]amino}-2-({[(9H-fluoren-9-yl)methoxy]carbonyl}amino)hexanoic acid, (2S)-6-({1-[2-(tert-butoxy)-2-oxoethyl]piperidin-4-yl}formamido)-2-({[(9H-fluoren-9-yl)methoxy]carbonyl}amino)hexanoic acid, (2R)-6-{1-[2-(tert-butoxy)-2-oxoethyl]piperidine-4-sulfonamido}-2-({[(9H-fluoren-9-yl)methoxy]carbonyl}amino)hexanoic acid, (2S)-3-[4-(2-{4-[2-(tert-butoxy)-2-oxoethyl]piperazin-1-yl}-2-oxoethoxy)phenyl]-2-({[(9H-fluoren-9-yl)methoxy]carbonyl}amino)propanoic acid, (2R)-6-({1-[2-(tert-butoxy)-2-oxoethyl]piperidin-4-yl}formamido)-2-({[(9H-fluoren-9-yl)methoxy]carbonyl}amino)hexanoic acid, (2R)-6-(2-{1-[2-(tert-butoxy)-2-oxoethyl]piperidin-4-yl}acetamido)-2-({[(9H-fluoren-9-yl)methoxy]carbonyl}amino)hexanoic acid, (2S)-6-{1-[2-(tert-butoxy)-2-oxoethyl]piperidine-4-sulfonamido}-2-({[(9H-fluoren-9-yl)methoxy]carbonyl}amino)hexanoic acid, (2S)-3-({1-[2-(tert-butoxy)-2-oxoethyl]piperidin-4-yl}formamido)-2-({[(9H-fluoren-9-yl)methoxy]carbonyl}amino)propanoic acid, (2R)-6-{4-[2-(tert-butoxy)-2-oxoethyl]piperazin-1-yl}-2-({[(9H-fluoren-9-yl)methoxy]carbonyl}amino)-6-oxohexanoic acid, (2R)-5-({1-[2-(tert-butoxy)-2-oxoethyl]piperidin-4-yl}carbamoyl)-2-({[(9H-fluoren-9-yl)methoxy]carbonyl}amino)pentanoic acid, (2R)-6-{[({1,3-bis[(2,2-dimethyl-1,3-dioxan-5-yl)methoxy]propan-2-yl}oxy)carbonyl]amino}-2-({[(9H-fluoren-9-yl)methoxy]carbonyl}amino)hexanoic acid, (2R)-2-({[(9H-fluoren-9-yl)methoxy]carbonyl}amino)-6-{4-[(2-methoxyethoxy)methyl]-2-oxo-3,6,9-trioxa-1-azadecan-1-yl}hexanoic acid, (2S)-6-{[({1,3-bis[(2,2-dimethyl-1,3-dioxan-5-yl)methoxy]propan-2-yl}oxy)carbonyl]amino}-2-({[(9H-fluoren-9-yl)methoxy]carbonyl}amino)hexanoic acid, (2S)-6-({5-[(tert-butoxy)carbonyl]pyridin-2-yl}amino)-2-({[(9H-fluoren-9-yl)methoxy]carbonyl}amino)hexanoic acid, (2S)-2-({[(9H-fluoren-9-yl)methoxy]carbonyl}(methyl)amino)-6-{[(prop-2-en-1-yloxy)carbonyl]amino}hexanoic acid, “(2S)-6-acetamido-2-({[(9H-fluoren-9-yl)methoxy]carbonyl}(methyl)amino)hexanoic acid, (2S)-2-({[(9H-fluoren-9-yl)methoxy]carbonyl}(methyl)amino)-4-{[(prop-2-en-1-yloxy)carbonyl]amino}butanoic acid, (2S)-3-acetamido-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-{[(prop-2-en-1-yloxy)carbonyl]amino}propanoic acid, (2S)-3-{1-[2-(tert-butoxy)-2-oxoethyl]piperidin-4-yl}-2-({[(9H-fluoren-9-yl)methoxy]carbonyl}amino)propanoic acid, (2S)-2-{1-[2-(tert-butoxy)-2-oxoethyl]piperidin-4-yl}-2-({[(9H-fluoren-9-yl)methoxy]carbonyl}amino)acetic acid, (2R)-3-{1-[2-(tert-butoxy)-2-oxoethyl]piperidin-4-yl}-2-({[(9H-fluoren-9-yl)methoxy]carbonyl}amino)propanoic acid, (2S)-3-{4-[({4-[2-(tert-butoxy)-2-oxoethyl]piperazine-1-carbonyl}amino)methyl]phenyl}-2-({[(9H-fluoren-9-yl)methoxy]carbonyl}amino)propanoic acid, (2R)-3-{4-[({4-[2-(tert-butoxy)-2-oxoethyl]piperazine-1-carbonyl}amino)methyl]phenyl}-2-({[(9H-fluoren-9-yl)methoxy]carbonyl}amino)propanoic acid, (2S)-3-{3-1[({4-[2-(tert-butoxy)-2-oxoethyl]piperazine-1-carbonyl}amino)methyl]phenyl}-2-({[(9H-fluoren-9-yl)methoxy]carbonyl}amino)propanoic acid, (2S)-3-(6-{[2-(tert-butoxy)-2-oxoethyl]amino}pyridin-3-yl)-2-({[(9H-fluoren-9-yl)methoxy]carbonyl}amino)propanoic acid, (2R)-3-{3-[(tert-butoxy)carbonyl]phenyl}-2-({[(9H-fluoren-9-yl)methoxy]carbonyl}amino)propanoic acid, (2S)-3-{1-[(tert-butoxy)carbonyl]-1H-pyrrolo[3,2-c]pyridin-3-yl}-2-({[(9H-fluoren-9-yl)methoxy]carbonyl}amino)propanoic acid, (2S)-2-({[(9H-fluoren-9-yl)methoxy]carbonyl}amino)-3-[1-(triphenylmethyl)-1H-pyrazol-3-yl]propanoic acid, (2S)-3-(3,4-dimethylphenyl)-2-({[(9H-fluoren-9-yl)methoxy]carbonyl}(methyl)amino)propanoic acid, (2S)-3-(3,4-dimethoxyphenyl)-2-({[(9H-fluoren-9-yl)methoxy]carbonyl}(methyl)amino)propanoic acid, (2S)-3-{1-[(tert-butoxy)carbonyl]-1H-indol-6-yl}-2-({[(9H-fluoren-9-yl)methoxy]carbonyl}amino)propanoic acid, (2S)-3-{1-[(tert-butoxy)carbonyl]-5-methyl-1H-imidazol-4-yl}-2-({[(9H-fluoren-9-yl)methoxy]carbonyl}amino)propanoic acid, (2S)-2-({[(9H-fluoren-9-yl)methoxy]carbonyl}amino)-3-(2-oxo-1,2-dihydropyridin-4-yl)propanoic acid, (2R)-3-(6-{[(tert-butoxy)carbonyl]amino}pyridin-3-yl)-2-({[(9H-fluoren-9-yl)methoxy]carbonyl}amino)propanoic acid, (2S)-2-({[(9H-fluoren-9-yl)methoxy]carbonyl}amino)-3-(6-hydroxy-1H-indol-3-yl)propanoic acid, (2S)-3-(2H-1,3-benzodioxol-5-yl)-2-({[(9H-fluoren-9-yl)methoxy]carbonyl}(methyl)amino)propanoic acid, (2S)-3-(4-ethylphenyl)-2-({[(9H-fluoren-9-yl)methoxy]carbonyl}(methyl)amino)propanoic acid, (2S)-2-({[(9H-fluoren-9-yl)methoxy]carbonyl}(methyl)amino)-4-(pyridin-3-yl)butanoic acid, (2S)-2-({[(9H-fluoren-9-yl)methoxy]carbonyl}(methyl)amino)-4-(pyridin-2-yl)butanoic acid, (2S)-2-({[(9H-fluoren-9-yl)methoxy]carbonyl}(methyl)amino)-4-(5-methoxypyridin-2-yl)butanoic acid, (2S)-2-({[(9H-fluoren-9-yl)methoxy]carbonyl}(methyl)amino)-4-(6-methoxypyridin-3-yl)butanoic acid, (2S)-2-({[(9H-fluoren-9-yl)methoxy]carbonyl}amino)-3-(1H-1,2,3-triazol-4-yl)propanoic acid, (2S)-2-({[(9H-fluoren-9-yl)methoxy]carbonyl}amino)-3-{4-[(prop-2-en-1-yloxy)carbonyl]phenyl}propanoic acid, (2S)-3-{6-[(tert-butoxy)carbonyl]pyridin-3-yl}-2-({[(9H-fluoren-9-yl)methoxy]carbonyl}amino)propanoic acid, (2S)-6-({bis[2-(tert-butoxy)-2-oxoethyl]carbamoyl}amino)-2-({[(9H-fluoren-9-yl)methoxy]carbonyl}amino)hexanoic acid, (2S)-2-({[(9H-fluoren-9-yl)methoxy]carbonyl}amino)-6-({4-[2-oxo-2-(prop-2-en-1-yloxy)ethyl]piperazine-1-carbonyl}amino)hexanoic acid, (2S)-2-({[(9H-fluoren-9-yl)methoxy]carbonyl}amino)-6[(4-{2-[(3-methylpentan-3-yl)oxy]-2-oxoethyl}piperazine-1-carbonyl)amino]hexanoic acid, (2S)-5-{4-[2-(tert-butoxy)-2-oxoethyl]piperazin-1-yl}-2-({[(9H-fluoren-9-yl)methoxy]carbonyl}amino)-5-oxopentanoic acid, (2R)-3-({1-[2-(tert-butoxy)-2-oxoethyl]piperidin-4-yl}formamido)-2-({[(9H-fluoren-9-yl)methoxy]carbonyl}amino)propanoic acid, (2S)-3-({1-[2-(tert-butoxy)-2-oxoethyl]piperidin-4-yl}formamido)-2-({[(9H-fluoren-9-yl)methoxy]carbonyl}(methyl)amino)propanoic acid, (2S)-6-{4-[2-(tert-butoxy)-2-oxoethyl]piperazin-1-yl}-2-({[(9H-fluoren-9-yl)methoxy]carbonyl}amino)-6-oxohexanoic acid, (2S)-4-{bis[2-(tert-butoxy)-2-oxoethyl]carbamoyl}-2-({[(9H-fluoren-9-yl)methoxy]carbonyl}amino)butanoic acid, (2S)-2-({[(9H-fluoren-9-yl)methoxy]carbonyl}amino)-3-{1-[2-oxo-2-(prop-2-en-1-yloxy)ethyl]piperidin-4-yl}propanoic acid, 1-[2-(tert-butoxy)-2-oxoethyl]-4-({[(9H-fluoren-9-yl)methoxy]carbonyl}amino)piperidine-4-carboxylic acid, (2S)-3-{1-[2-(tert-butoxy)-2-oxoethyl]piperidin-4-yl}-2-({[(9H-fluoren-9-yl)methoxy]carbonyl}(methyl)amino)propanoic acid, (2R)-3-{4-[({4-[2-(tert-butoxy)-2-oxoethyl]piperazine-1-carbonyl}amino)methyl]phenyl}-2-({[(9H-fluoren-9-yl)methoxy]carbonyl}(methyl)amino)propanoic acid, (2R)-3-{4-[({4-[2-(tert-butoxy)-2-oxoethyl]piperazine-1-carbonyl}amino)methyl]phenyl}-2-({[(9H-fluoren-9-yl)methoxy]carbonyl}(methyl)amino)propanoic acid, (2S)-3-[3-(tert-butoxy)phenyl]-2-({[(9H-fluoren-9-yl)methoxy]carbonyl}(methyl)amino)propanoic acid, (2S)-3-{4-[2-({1-[2-(tert-butoxy)-2-oxoethyl]piperidin-4-yl}formamido)ethoxy]phenyl}-2-({[(9H-fluoren-9-yl)methoxy]carbonyl}amino)propanoic acid, (2S)-3-[4-(2-{[2-(tert-butoxy)-2-oxoethyl][(tert-butoxy)carbonyl]amino}ethoxy)phenyl]-2-({[(9H-fluoren-9-yl)methoxy]carbonyl}amino)propanoic acid, (2R)-6-[({1-[2-(tert-butoxy)-2-oxoethyl]azetidin-3-yl}carbamoyl)amino]-2-({[(9H-fluoren-9-yl)methoxy]carbonyl}amino)hexanoic acid, (2R)-6-[({1-[2-(tert-butoxy)-2-oxoethyl]piperidin-4-yl}carbamoyl)oxy]-2-({[(9H-fluoren-9-yl)methoxy]carbonyl}amino)hexanoic acid, (S)-2-((((9H-fluoren-9-yl)methoxy)carbonyl)amino)-3-((2-(tert-butoxy)-2-oxoethyl)(methyl)amino)propanoic acid, (2S)-2-({[(9H-fluoren-9-yl)methoxy]carbonyl}amino)-3-{methyl[2-oxo-2-(prop-2-en-1-yloxy)ethyl]amino}propanoic acid, and (2S)-4-({4-[2-(tert-butoxy)-2-oxoethyl]piperazine-1-carbonyl}amino)-2-({[(9H-fluoren-9-yl)methoxy]carbonyl}amino)butanoic acid, (2S)-2-({[(9H-fluoren-9-yl)methoxy]carbonyl}(methyl)amino)-6-(N-methylacetamido)hexanoic acid, 2-({[(9H-fluoren-9-yl)methoxy]carbonyl}[4-(N-methylmethanesulfonamido)butyl]amino)acetic acid, 2-({[(9H-fluoren-9-yl)methoxy]carbonyl}(4-methanesulfonamidobutyl)amino)acetic acid, 2-({[(9H-fluoren-9-yl)methoxy]carbonyl}(3-methanesulfonamidopropyl)amino)acetic acid, 2-({[(9H-fluoren-9-yl)methoxy]carbonyl}[3-(N-methylmethanesulfonamido)propyl]amino)acetic acid, 2-[(4-acetamidobutyl)({[(9H-fluoren-9-yl)methoxy]carbonyl})amino]acetic acid, 2-({[(9H-fluoren-9-yl)methoxy]carbonyl}[3-(N-methylacetamido)propyl]amino)acetic acid, 2-[(3-{[(tert-butoxy)carbonyl](methyl)amino}propyl)({[(9H-fluoren-9-yl)methoxy]carbonyl})amino]acetic acid, (2S)-3-{1-[(tert-butoxy)carbonyl]-5-phenoxy-1H-indol-3-yl}-2-({[(9H-fluoren-9-yl)methoxy]carbonyl}amino)propanoic acid, (2S)-2-({[(9H-fluoren-9-yl)methoxy]carbonyl}amino)-3-[6-(pyridin-3-yl)-1H-indol-3-yl]propanoic acid, (2S)-2-({[(9H-fluoren-9-yl)methoxy]carbonyl}amino)-3-[6-(pyridin-4-yl)-1H-indol-3-yl]propanoic acid, (2S)-2-({[(9H-fluoren-9-yl)methoxy]carbonyl}amino)-3-[6-(pyrimidin-5-yl)-1H-indol-3-yl]propanoic acid, (2S)-3-{1-[(tert-butoxy)carbonyl]-6-(1-methyl-1H-pyrazol-4-yl)-1H-indol-3-yl}-2-({[(9H-fluoren-9-yl)methoxy]carbonyl}amino)propanoic acid, (2S)-3-(3-carbamoyl-4-hydroxyphenyl)-2-({[(9H-fluoren-9-yl)methoxy]carbonyl}amino)propanoic acid, (2S)-3-[3-(dimethylcarbamoyl)-4-hydroxyphenyl]-2-({[(9H-fluoren-9-yl)methoxy]carbonyl}amino)propanoic acid, (2S)-2-({[(9H-fluoren-9-yl)methoxy]carbonyl}amino)-3-[4-(2-{1[(prop-2-en-1-yloxy)carbonyl]amino}ethoxy)phenyl]propanoic acid, (2S)-3-[4-(2-acetamidoethoxy)phenyl]-2-({[(9H-fluoren-9-yl)methoxy]carbonyl}amino)propanoic acid, (2S)-3-(3-carbamoylpyridin-2-yl)-2-({[(9H-fluoren-9-yl)methoxy]carbonyl}amino)propanoic acid, (2S)-2-({[(9H-fluoren-9-yl)methoxy]carbonyl}amino)-3-(6-phenylpyridin-3-yl)propanoic acid, (2R)-2-({[(9H-fluoren-9-yl)methoxy]carbonyl}amino)-3-(6-phenylpyridin-3-yl)propanoic acid, (2S)-3-(2-carbamoylpyridin-4-yl)-2-({[(9H-fluoren-9-yl)methoxy]carbonyl}amino)propanoic acid, (2S)-3-(3-carbamoylpyridin-4-yl)-2-({[(9H-fluoren-9-yl)methoxy]carbonyl}amino)propanoic acid, (2S)-2-({[(9H-fluoren-9-yl)methoxy]carbonyl}amino)-3-(1H-indazol-5-yl)propanoic acid, (2R)-2-({[(9H-fluoren-9-yl)methoxy]carbonyl}amino)-3-(1H-indazol-5-yl)propanoic acid, (2S)-3-(1-acetylpiperidin-4-yl)-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-{1-[(prop-2-en-1-yloxy)carbonyl]piperidin-4-yl}propanoic acid, (2S)-3-{1-[(tert-butoxy)carbonyl]piperidin-4-yl}-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-{1-[1-(triphenylmethyl)-1H-pyrazole-4-carbonyl]piperidin-4-yl}propanoic acid, (2S)-2-({[(9H-fluoren-9-yl)methoxy]carbonyl}(methyl)amino)-3-{1-[3-(triphenylmethoxy)propanoyl]piperidin-4-yl}propanoic acid, (2S)-3-{1-[2-(tert-butoxy)acetyl]piperidin-4-yl}-2-({[(9H-fluoren-9-yl)methoxy]carbonyl}(methyl)amino)propanoic acid, (2S)-2-({[(9H-fluoren-9-yl)methoxy]carbonyl}amino)-7-methoxyheptanoic acid, (2S)-3-[3′-(tert-butoxy)-[1,1′-biphenyl]-4-yl]-2-({[(9H-fluoren-9-yl)methoxy]carbonyl}amino)propanoic acid, (2S)-2-({[(9H-fluoren-9-yl)methoxy]carbonyl}amino)-4-methanesulfonamidobutanoic acid, (2R)-2-({[(9H-fluoren-9-yl)methoxy]carbonyl}(methyl)amino)-3-{[(prop-2-en-1-yloxy)carbonyl]amino}propanoic acid, (2S)-3-{[(tert-butoxy)carbonyl]amino}-2-({[(9H-fluoren-9-yl)methoxy]carbonyl}(methyl)amino)propanoic acid, (2R)-2-({[(9H-fluoren-9-yl)methoxy]carbonyl}amino)-3-{3-[2-oxo-2-(prop-2-en-1-yloxy)ethyl]phenyl}propanoic acid, (2S)-2-({[(9H-fluoren-9-yl)methoxy]carbonyl}amino)-3-{3-[2-oxo-2-(prop-2-en-1-yloxy)ethyl]phenyl}propanoic acid, (2R)-3-{3-[2-(tert-butoxy)-2-oxoethyl]phenyl}-2-({[(9H-fluoren-9-yl)methoxy]carbonyl}amino)propanoic acid, (2S)-3-{3-[2-(tert-butoxy)-2-oxoethyl]phenyl}-2-({[(9H-fluoren-9-yl)methoxy]carbonyl}amino)propanoic acid, (2R)-2-({[(9H-fluoren-9-yl)methoxy]carbonyl}amino)-3-{3-[2-oxo-2-(prop-2-en-1-yloxy)ethoxy]phenyl}propanoic acid, (2S)-2-({[(9H-fluoren-9-yl)methoxy]carbonyl}amino)-3-{3-[2-oxo-2-(prop-2-en-1-yloxy)ethoxy]phenyl}propanoic acid, (2R)-3-{3-[2-(tert-butoxy)-2-oxoethoxy]phenyl}-2-({[(9H-fluoren-9-yl)methoxy]carbonyl}amino)propanoic acid, (2S)-3-{3-[2-(tert-butoxy)-2-oxoethoxy]phenyl}-2-({[(9H-fluoren-9-yl)methoxy]carbonyl}amino)propanoic acid, (2S)-3-[3-(acetamidomethyl)phenyl]-2-({[(9H-fluoren-9-yl)methoxy]carbonyl}(methyl)amino)propanoic acid, (2S)-3-(3-carbamoylphenyl)-2-({[(9H-fluoren-9-yl)methoxy]carbonyl}(methyl)amino)propanoic acid, (2R)-3-(3-carbamoylphenyl)-2-({[(9H-fluoren-9-yl)methoxy]carbonyl}amino)propanoic acid, (2S)-3-[3-(benzylcarbamoyl)phenyl]-2-({[(9H-fluoren-9-yl)methoxy]carbonyl}amino)propanoic acid, (2S)-3-[3-(dimethylcarbamoyl)phenyl]-2-({[(9H-fluoren-9-yl)methoxy]carbonyl}amino)propanoic acid, (2S)-2-({[(9H-fluoren-9-yl)methoxy]carbonyl}(methyl)amino)-3-(3-{[(prop-2-en-1-yloxy)carbonyl]amino}phenyl)propanoic acid, (2S)-3-(4-carbamoylphenyl)-2-({[(9H-fluoren-9-yl)methoxy]carbonyl}(methyl)amino)propanoic acid, (2S)-3-[4-(dimethylcarbamoyl)phenyl]-2-({[(9H-fluoren-9-yl)methoxy]carbonyl}amino)propanoic acid, (2S)-2-({[(9H-fluoren-9-yl)methoxy]carbonyl}amino)-3-{4-[(2,5,8,11-tetraoxatridecan-13-yl)carbamoyl]phenyl}propanoic acid, (2S)-4-(2-{[(tert-butoxy)carbonyl]amino}pyridin-3-yl)-2-({[(9H-fluoren-9-yl)methoxy]carbonyl}amino)butanoic acid, (2S)-6-(carbamoylamino)-2-({[(9H-fluoren-9-yl)methoxy]carbonyl}(methyl)amino)hexanoic acid, (2S)-4-{4-[(tert-butoxy)carbonyl]phenyl}-2-({[(9H-fluoren-9-yl)methoxy]carbonyl}amino)butanoic acid, (2S)-2-({[(9H-fluoren-9-yl)methoxy]carbonyl}(methyl)amino)-6-[(2H-1,2,3,4-tetrazol-5-yl)amino]hexanoic acid, (2S)-6-{[(tert-butoxy)carbonyl](1H-1,2,3,4-tetrazol-5-yl)amino}-2-({[(9H-fluoren-9-yl)methoxy]carbonyl}(methyl)amino)hexanoic acid, (2S)-6-(2-acetamidoacetamido)-2-({[(9H-fluoren-9-yl)methoxy]carbonyl}(methyl)amino)hexanoic acid, (2S)-6-[4-(tert-butoxy)-4-oxobutanamido]-2-({[(9H-fluoren-9-yl)methoxy]carbonyl}(methyl)amino)hexanoic acid, (2S)-6-(cyclopropylformamido)-2-({[(9H-fluoren-9-yl)methoxy]carbonyl}(methyl)amino)hexanoic acid, (2S)-2-({[(9H-fluoren-9-yl)methoxy]carbonyl}(methyl)amino)-6-propanamidohexanoic acid, (2S)-2-({[(9H-fluoren-9-yl)methoxy]carbonyl}(methyl)amino)-6-[3-(triphenylmethoxy)propanamido]hexanoic acid, (2S)-2-({[(9H-fluoren-9-yl)methoxy]carbonyl}(methyl)amino)-6-(2-methylpropanamido)hexanoic acid, (2S)-2-({[(9H-fluoren-9-yl)methoxy]carbonyl}(methyl)amino)-6-(3-methoxypropanamido)hexanoic acid, (2S)-2-({[(9H-fluoren-9-yl)methoxy]carbonyl}(methyl)amino)-6-[(2S)-2-(triphenylmethoxy)propanamido]hexanoic acid, (2S)-2-({[(9H-fluoren-9-yl)methoxy]carbonyl}(methyl)amino)-6-[(2R)-2-(triphenylmethoxy)propanamido]hexanoic acid, (2S)-2-({[(9H-fluoren-9-yl)methoxy]carbonyl}(methyl)amino)-6-(phenylformamido)hexanoic acid, (2S)-6-[2-(tert-butoxy)acetamido]-2-({[(9H-fluoren-9-yl)methoxy]carbonyl}(methyl)amino)hexanoic acid, (2S)-2-({[(9H-fluoren-9-yl)methoxy]carbonyl}(methyl)amino)-6-(2-methoxyacetamido)hexanoic acid, (2S)-2-({[(9H-fluoren-9-yl)methoxy]carbonyl}(methyl)amino)-6-(morpholin-4-yl)hexanoic acid, (2S)-2-({[(9H-fluoren-9-yl)methoxy]carbonyl}(methyl)amino)-6-methanesulfonamidohexanoic acid, (2S)-2-({[(9H-fluoren-9-yl)methoxy]carbonyl}(methyl)amino)-6-(2-oxoimidazolidin-1-yl)hexanoic acid, (2S)-2-({[(9H-fluoren-9-yl)methoxy]carbonyl}(methyl)amino)-6-(2-oxopyrrolidin-1-yl)hexanoic acid, (2S)-2-({[(9H-fluoren-9-yl)methoxy]carbonyl}(methyl)amino)-6-(2-oxo-1,3-diazinan-1-yl)hexanoic acid, (2S)-3-{4′-cyano-[1,1′-biphenyl]-3-yl}-2-({[(9H-fluoren-9-yl)methoxy]carbonyl}amino)propanoic acid, (2S)-2-({[(9H-fluoren-9-yl)methoxy]carbonyl}(methyl)amino)-6-(1H-1,2,3-triazol-1-yl)hexanoic acid, (2S)-5-acetamido-2-({[(9H-fluoren-9-yl)methoxy]carbonyl}(methyl)amino)pentanoic acid, (2S)-2-({[(9H-fluoren-9-yl)methoxy]carbonyl}amino)-5-methanesulfonamidopentanoic acid, (2S)-2-({[(9H-fluoren-9-yl)methoxy]carbonyl}amino)-4-{[(pyridin-4-yl)methyl]carbamoyl}butanoic acid hydrochloride, (2S)-2-({[(9H-fluoren-9-yl)methoxy]carbonyl}amino)-4-{[(pyridin-4-yl)methyl]carbamoyl}butanoic acid, (2S)-2-({[(9H-fluoren-9-yl)methoxy]carbonyl}amino)-4-{[(1s,3s)-3-{[(tert-butoxy)carbonyl]amino}cyclobutyl]carbamoyl}butanoic acid, (2S)-4-[(carbamoylmethyl)carbamoyl]-2-({[(9H-fluoren-9-yl)methoxy]carbonyl}(methyl)amino)butanoic acid, (2S)-2-({[(9H-fluoren-9-yl)methoxy]carbonyl}amino)-4-{[(pyrimidin-5-yl)methyl]carbamoyl}butanoic acid, (2S)-4-{[(3S)-1-[(tert-butoxy)carbonyl]pyrrolidin-3-yl]carbamoyl}-2-({[(9H-fluoren-9-yl)methoxy]carbonyl}amino)butanoic acid, (2S)-2-({[(9H-fluoren-9-yl)methoxy]carbonyl}amino)-3-(5,6,7,8-tetrahydronaphthalen-2-yl)propanoic acid, (2S)-3-{5-[2-(tert-butoxy)-2-oxoethoxy]-1-[(tert-butoxy)carbonyl]-1H-indol-3-yl}-2-({[(9H-fluoren-9-yl)methoxy]carbonyl}amino)propanoic acid, (2S)-2-({[(9H-fluoren-9-yl)methoxy]carbonyl}amino)-3-{3′-methanesulfonyl-[1,1′-biphenyl]-4-yl}propanoic acid, (2S)-2-({[(9H-fluoren-9-yl)methoxy]carbonyl}amino)-3-methanesulfonamidopropanoic acid, (2S)-3-(2-acetamidoacetamido)-2-({[(9H-fluoren-9-yl)methoxy]carbonyl}(methyl)amino)propanoic acid, (2S)-2-({[(9H-fluoren-9-yl)methoxy]carbonyl}amino)-3-[3-({[(prop-2-en-1-yloxy)carbonyl]amino}methyl)phenyl]propanoic acid, (2S)-2-({[(9H-fluoren-9-yl)methoxy]carbonyl}(methyl)amino)-3-(4-{[(prop-2-en-1-yloxy)carbonyl]amino}phenyl)propanoic acid, (2S)-2-({[(9H-fluoren-9-yl)methoxy]carbonyl}amino)-3-(1,2-oxazol-4-yl)propanoic acid, (2S)-5-(tert-butoxy)-2-({[(9H-fluoren-9-yl)methoxy]carbonyl}(methyl)amino)-5-oxopentanoic acid, (2S)-2-({[(9H-fluoren-9-yl)methoxy]carbonyl}(methyl)amino)-4-[(3-methylpentan-3-yl)oxy]-4-oxobutanoic acid, (2S)-2-({[(9H-fluoren-9-yl)methoxy]carbonyl}(methyl)amino)-3-(triphenylmethoxy)propanoic acid, (2S)-3-(3-chlorophenyl)-2-({[(9H-fluoren-9-yl)methoxy]carbonyl}amino)propanoic acid, (2S)-2-({[(9H-fluoren-9-yl)methoxy]carbonyl}amino)-4-phenylbutanoic acid, (2S)-2-({[(9H-fluoren-9-yl)methoxy]carbonyl}amino)-4-(4-fluorophenyl)butanoic acid, (2S)-4-(4-{[(tert-butoxy)carbonyl]amino}phenyl)-2-({[(9H-fluoren-9-yl)methoxy]carbonyl}amino)butanoic acid, (2S)-2-({[(9H-fluoren-9-yl)methoxy]carbonyl}(methyl)amino)-3-(6-oxo-1,6-dihydropyridin-3-yl)propanoic acid, (2S)-2-({[(9H-fluoren-9-yl)methoxy]carbonyl}(methyl)amino)-3-(4-methoxyphenyl)propanoic acid, (2S)-2-({[(9H-fluoren-9-yl)methoxy]carbonyl}amino)-3-(6-oxo-1,6-dihydropyridin-3-yl)propanoic acid, (2S)-3-(5,6-dimethoxypyridin-3-yl)-2-({[(9H-fluoren-9-yl)methoxy]carbonyl}amino)propanoic acid, (2S)-2-({[(9H-fluoren-9-yl)methoxy]carbonyl}amino)-3-[2-(methylcarbamoyl)pyridin-4-yl]propanoic acid, (2S)-2-({[(9H-fluoren-9-yl)methoxy]carbonyl}amino)-3-(4-methanesulfonylphenyl)propanoic acid, (2S)-2-({[(9H-fluoren-9-yl)methoxy]carbonyl}(methyl)amino)-3-{4-[(prop-2-en-1-yloxy)carbonyl]phenyl}propanoic acid, (2S)-3-(4-acetamidophenyl)-2-({[(9H-fluoren-9-yl)methoxy]carbonyl}amino)propanoic acid, (2S)-2-({[(9H-fluoren-9-yl)methoxy]carbonyl}amino)-3-(6-methoxypyridin-3-yl)propanoic acid, (2S)-3-{4-[(tert-butoxy)carbonyl]phenyl}-2-({[(9H-fluoren-9-yl)methoxy]carbonyl}(methyl)amino)propanoic acid, (2S)-3-{3-[(tert-butoxy)carbonyl]phenyl}-2-({[(9H-fluoren-9-yl)methoxy]carbonyl}(methyl)amino)propanoic acid, (2S)-3-{5-[(tert-butoxy)carbonyl]pyridin-3-yl}-2-({[(9H-fluoren-9-yl)methoxy]carbonyl}amino)propanoic acid, (2S)-4-{3-[(tert-butoxy)carbonyl]phenyl}-2-({[(9H-fluoren-9-yl)methoxy]carbonyl}amino)butanoic acid, (2S)-2-({[(9H-fluoren-9-yl)methoxy]carbonyl}amino)-3-(2-methoxypyridin-4-yl)propanoic acid, (2S)-3-{3-[(tert-butoxy)carbonyl]-4-hydroxyphenyl}-2-({[(9H-fluoren-9-yl)methoxy]carbonyl}amino)propanoic acid, (2S)-3-[4-(tert-butoxy)-3-methylphenyl]-2-({[(9H-fluoren-9-yl)methoxy]carbonyl}amino)propanoic acid, (2S)-3-(2-{[(tert-butoxy)carbonyl]amino}pyridin-4-yl)-2-({[(9H-fluoren-9-yl)methoxy]carbonyl}(methyl)amino)propanoic acid, (2S)-3-[4-({[(tert-butoxy)carbonyl]amino}methyl)phenyl]-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-[4-({[(prop-2-en-1-yloxy)carbonyl]amino}methyl)phenyl]propanoic acid, (2S)-3-{3-[(3-{[(tert-butoxy)carbonyl]amino}propyl)carbamoyl]phenyl}-2-({[(9H-fluoren-9-yl)methoxy]carbonyl}(methyl)amino)propanoic acid, (2S)-3-{3-[(2-{1[(tert-butoxy)carbonyl]amino}ethyl)carbamoyl]phenyl}-2-({[(9H-fluoren-9-yl)methoxy]carbonyl}(methyl)amino)propanoic acid, (2S)-3-(6-{[(tert-butoxy)carbonyl](methyl)amino}pyridin-3-yl)-2-({[(9H-fluoren-9-yl)methoxy]carbonyl}amino)propanoic acid, (2S)-3-[3-({[(tert-butoxy)carbonyl]amino}methyl)phenyl]-2-({[(9H-fluoren-9-yl)methoxy]carbonyl}(methyl)amino)propanoic acid, (2S,4R)-4-(tert-butoxy)-1-{[(9H-fluoren-9-yl)methoxy]carbonyl}pyrrolidine-2-carboxylic acid, (2R,4S)-4-{[(tert-butoxy)carbonyl]amino}-1-{[(9H-fluoren-9-yl)methoxy]carbonyl}pyrrolidine-2-carboxylic acid, (2R,4R)-1-{[(9H-fluoren-9-yl)methoxy]carbonyl}-4-hydroxypyrrolidine-2-carboxylic acid, (2S)-2-({[(9H-fluoren-9-yl)methoxy]carbonyl}(methyl)amino)pentanoic acid, (2S)-3-cyclobutyl-2-({[(9H-fluoren-9-yl)methoxy]carbonyl}amino)propanoic acid, (2S)-3-cyclopentyl-2-({[(9H-fluoren-9-yl)methoxy]carbonyl}amino)propanoic acid, (2S)-4-ethyl-2-({[(9H-fluoren-9-yl)methoxy]carbonyl}amino)hexanoic acid, (2S)-2-({[(9H-fluoren-9-yl)methoxy]carbonyl}amino)-7,7,7-trifluoroheptanoic acid, (2S)-4-cyclobutyl-2-({[(9H-fluoren-9-yl)methoxy]carbonyl}amino)butanoic acid, (2S)-4-cyclopentyl-2-({[(9H-fluoren-9-yl)methoxy]carbonyl}amino)butanoic acid, (2S)-2-({[(9H-fluoren-9-yl)methoxy]carbonyl}amino)-3-(1-methyl-1H-pyrazol-4-yl)propanoic acid, (2S)-2-({[(9H-fluoren-9-yl)methoxy]carbonyl}amino)-3-(1,3-oxazol-5-yl)propanoic acid, (2S)-2-({[(9H-fluoren-9-yl)methoxy]carbonyl}amino)-3-(1,3-oxazol-4-yl)propanoic acid, (2S)-2-({[(9H-fluoren-9-yl)methoxy]carbonyl}amino)-3-[1-(triphenylmethyl)-1H-pyrazol-4-yl]propanoic acid, (2S)-2-({[(9H-fluoren-9-yl)methoxy]carbonyl}amino)-3-(5-hydroxy-1H-indol-3-yl)propanoic acid, (2S)-2-({[(9H-fluoren-9-yl)methoxy]carbonyl}amino)-3-(naphthalen-1-yl)propanoic acid, (2S)-2-({[(9H-fluoren-9-yl)methoxy]carbonyl}(methyl)amino)-3-(naphthalen-2-yl)propanoic acid, (2S)-3-{1-[(tert-butoxy)carbonyl]-7-methoxy-1H-indol-3-yl}-2-({[(9H-fluoren-9-yl)methoxy]carbonyl}amino)propanoic acid, (2S)-2-({[(9H-fluoren-9-yl)methoxy]carbonyl}amino)-3-(quinolin-6-yl)propanoic acid, (2S)-3-{1-[(tert-butoxy)carbonyl]-5-hydroxy-1H-indol-3-yl}-2-({[(9H-fluoren-9-yl)methoxy]carbonyl}amino)propanoic acid, (2S)-2-({[(9H-fluoren-9-yl)methoxy]carbonyl}amino)-3-{1H-pyrrolo[3,2-c]pyridin-3-yl}propanoic acid, (2S)-2-({[(9H-fluoren-9-yl)methoxy]carbonyl}amino)-3-{1-methyl-1H-pyrrolo[2,3-b]pyridin-3-yl}propanoic acid, (2S)-3-(1-{2-[(4-{[1-(4,4-dimethyl-2,6-dioxocyclohexylidene)-3-methylbutyl]amino}phenyl)methoxy]-2-oxoethyl}-1H-indol-3-yl)-2-({[(9H-fluoren-9-yl)methoxy]carbonyl}amino)propanoic acid, (2S)-2-({[(9H-fluoren-9-yl)methoxy]carbonyl}(methyl)amino)-3-(1H-indazol-5-yl)propanoic acid, (2S)-3-[5-(benzyloxy)-1H-indol-3-yl]-2-({[(9H-fluoren-9-yl)methoxy]carbonyl}amino)propanoic acid, (2S)-3-{1-[(tert-butoxy)carbonyl]-5-[(4-methoxy phenyl)methoxy]-1H-indol-3-yl}-2-({[(9H-fluoren-9-yl)methoxy]carbonyl}amino)propanoic acid, (2S,3S)-3-(tert-butoxy)-2-({[(9H-fluoren-9-yl)methoxy]carbonyl}amino)butanoic acid, (2S)-2-({[(9H-fluoren-9-yl)methoxy]carbonyl}amino)-3-(oxan-4-yl)propanoic acid (2S)-2-({[(9H-fluoren-9-yl)methoxy]carbonyl}amino)-3-[(1r,4r)-4-methoxycyclohexyl]propanoic acid, (2S)-2-({[(9H-fluoren-9-yl)methoxy]carbonyl}amino)-3-[(1r,4r)-4-hydroxycyclohexyl]propanoic acid, (2S)-3-butoxy-2-({[(9H-fluoren-9-yl)methoxy]carbonyl}amino)propanoic acid, (2S)-2-({[(9H-fluoren-9-yl)methoxy]carbonyl}amino)-3-(pentyloxy)propanoic acid, (2S)-2-({[(9H-fluoren-9-yl)methoxy]carbonyl}amino)-4-propoxybutanoic acid, (2S)-4-ethoxy-2-({[(9H-fluoren-9-yl)methoxy]carbonyl}amino)butanoic acid, (2S)-4-butoxy-2-({[(9H-fluoren-9-yl)methoxy]carbonyl}amino)butanoic acid, (2S)-3-(cyclopentyloxy)-2-({[(9H-fluoren-9-yl)methoxy]carbonyl}amino)propanoic acid, (2S)-3-(cyclobutylmethoxy)-2-({[(9H-fluoren-9-yl)methoxy]carbonyl}amino)propanoic acid, (2S)-4-(cyclopentyloxy)-2-({[(9H-fluoren-9-yl)methoxy]carbonyl}amino)butanoic acid, (2S)-2-({[(9H-fluoren-9-yl)methoxy]carbonyl}amino)-4-(propan-2-yloxy)butanoic acid, (2S)-2-({[(9H-fluoren-9-yl)methoxy]carbonyl}amino)-3-[methyl(phenyl)amino]propanoic acid hydrochloride, (2S)-4-acetamido-2-({[(9H-fluoren-9-yl)methoxy]carbonyl}amino)butanoic acid, (2R)-3-{1-[(tert-butoxy)carbonyl]piperidin-4-yl}-2-({[(9H-fluoren-9-yl)methoxy]carbonyl}amino)propanoic acid, (2R)-4-{[(tert-butoxy)carbonyl]amino}-2-({[(9H-fluoren-9-yl)methoxy]carbonyl}amino)butanoic acid, (2R)-7-{[(tert-butoxy)carbonyl]amino}-2-({[(9H-fluoren-9-yl)methoxy]carbonyl}amino)heptanoic acid, (2S)-2-({[(9H-fluoren-9-yl)methoxy]carbonyl}amino)-7-{[(prop-2-en-1-yloxy)carbonyl]amino}heptanoic acid, (2R)-2-({[(9H-fluoren-9-yl)methoxy]carbonyl}amino)-7-{[(prop-2-en-1-yloxy)carbonyl]amino}heptanoic acid, (2S)-2-({[(9H-fluoren-9-yl)methoxy]carbonyl}amino)-3-(1-methanesulfonylpiperidin-4-yl)propanoic acid, (2R)-5-(carbamoylamino)-2-({[(9H-fluoren-9-yl)methoxy]carbonyl}amino)pentanoic acid, (2S)-6-(carbamoylamino)-2-({[(9H-fluoren-9-yl)methoxy]carbonyl}amino)hexanoic acid, (2S)-2-({[(9H-fluoren-9-yl)methoxy]carbonyl}(methyl)amino)-6-oxo-6-(prop-2-en-1-yloxy)hexanoic acid, (2S)-6-(tert-butoxy)-2-({[(9H-fluoren-9-yl)methoxy]carbonyl}(methyl)amino)-6-oxohexanoic acid, (2S)-5-(dimethylcarbamoyl)-2-({[(9H-fluoren-9-yl)methoxy]carbonyl}(methyl)amino)pentanoic acid, (2R)-2-({[(9H-fluoren-9-yl)methoxy]carbonyl}(methyl)amino)-4-[(3-methylpentan-3-yl)oxy]-4-oxobutanoic acid, (2S)-3-(3-carbamoylphenyl)-2-({[(9H-fluoren-9-yl)methoxy]carbonyl}amino)propanoic acid, (2S)-3-(4-carbamoylphenyl)-2-({[(9H-fluoren-9-yl)methoxy]carbonyl}amino)propanoic acid, (2R)-3-(4-carbamoylphenyl)-2-({[(9H-fluoren-9-yl)methoxy]carbonyl}(methyl)amino)propanoic acid, (2R)-3-(3-carbamoylphenyl)-2-({[(9H-fluoren-9-yl)methoxy]carbonyl}(methyl)amino)propanoic acid, (2S)-3-(4-{[(tert-butoxy)carbonyl]amino}phenyl)-2-({[(9H-fluoren-9-yl)methoxy]carbonyl}(methyl)amino)propanoic acid, (2R)-2-({[(9H-fluoren-9-yl)methoxy]carbonyl}amino)-3-[3-({[(prop-2-en-1-yloxy)carbonyl]amino}methyl)phenyl]propanoic acid, (2S)-3-[4-(acetamidomethyl)phenyl]-2-({[(9H-fluoren-9-yl)methoxy]carbonyl}(methyl)amino)propanoic acid, 2-({[(9H-fluoren-9-yl)methoxy]carbonyl}[2-(N-methylmethanesulfonamido)ethyl]amino)acetic acid, (2S)-4-(carbamoylamino)-2-({[(9H-fluoren-9-yl)methoxy]carbonyl}amino)butanoic acid, (2S)-5-(cyclohexylformamido)-2-({[(9H-fluoren-9-yl)methoxy]carbonyl}amino)pentanoic acid, (2S)-3-{3-[(3-{[(tert-butoxy)carbonyl]amino}propyl)carbamoyl]phenyl}-2-({[(9H-fluoren-9-yl)methoxy]carbonyl}amino)propanoic acid, (2S)-3-(7-chloro-1-methyl-1H-indol-3-yl)-2-({[(9H-fluoren-9-yl)methoxy]carbonyl}amino)propanoic acid, (2S)-2-({[(9H-fluoren-9-yl)methoxy]carbonyl}amino)-3-[4-(1-methyl-1H-pyrazol-4-yl)phenyl]propanoic acid, (2S)-2-({[(9H-fluoren-9-yl)methoxy]carbonyl}amino)-3-[4-(pyridin-3-yl)phenyl]propanoic acid hydrochloride, (2S)-2-({[(9H-fluoren-9-yl)methoxy]carbonyl}(methyl)amino)-3-(isoquinolin-7-yl)propanoic acid, (2S)-2-({[(9H-fluoren-9-yl)methoxy]carbonyl}amino)-3-(2-phenylpyridin-4-yl)propanoic acid, (2S)-2-({[(9H-fluoren-9-yl)methoxy]carbonyl}amino)-3-(quinolin-5-yl)propanoic acid, (2S)-3-{3-[(2-{[(tert-butoxy)carbonyl]amino}ethyl)carbamoyl]phenyl}-2-({[(9H-fluoren-9-yl)methoxy]carbonyl}amino)propanoic acid, 2-({[(9H-fluoren-9-yl)methoxy]carbonyl}({2-[(triphenylmethyl)carbamoyl]ethyl})amino)acetic acid, (2S)-2-({[(9H-fluoren-9-yl)methoxy]carbonyl}amino)-3-[4-(pyridin-2-yl)phenyl]propanoic acid hydrochloride, (2S)-4-[(2-carbamoylethyl)carbamoyl]-2-({[(9H-fluoren-9-yl)methoxy]carbonyl}amino)butanoic acid, (2S)-3-[4-(1-acetylpiperidin-4-yl)phenyl]-2-({[(9H-fluoren-9-yl)methoxy]carbonyl}amino)propanoic acid, (2S)-2-({[(9H-fluoren-9-yl)methoxy]carbonyl}(methyl)amino)-3-(quinolin-7-yl)propanoic acid, (2S)-2-({[(9H-fluoren-9-yl)methoxy]carbonyl}amino)-4-oxo-4-(pyrrolidin-1-yl)butanoic acid, (2S)-3-[1-(3-{[(tert-butoxy)carbonyl]amino}propyl)-1H-indol-3-yl]-2-({[(9H-fluoren-9-yl)methoxy]carbonyl}amino)propanoic acid, 2-[(2-{[1,1′-biphenyl]-4-yl}ethyl)({[(9H-fluoren-9-yl)methoxy]carbonyl})amino]acetic acid, (2S)-3-{1-[(tert-butoxy)carbonyl]-5,6-dichloro-1H-indol-3-yl}-2-({[(9H-fluoren-9-yl)methoxy]carbonyl}amino)propanoic acid, (2S)-3-(4-{1-[(tert-butoxy)carbonyl]piperidin-4-yl}phenyl)-2-({[(9H-fluoren-9-yl)methoxy]carbonyl}amino)propanoic acid, (2S)-2-({[(9H-fluoren-9-yl)methoxy]carbonyl}amino)-3-(4-{1-[(prop-2-en-1-yloxy)carbonyl]piperidin-4-yl}phenyl)propanoic acid, (2S)-3-(6-carbamoylpyridin-3-yl)-2-({[(9H-fluoren-9-yl)methoxy]carbonyl}amino)propanoic acid, (2S)-3-{1-[(tert-butoxy)carbonyl]-1H-pyrrolo[3,2-b]pyridin-3-yl}-2-({[(9H-fluoren-9-yl)methoxy]carbonyl}amino)propanoic acid, (2R)-2-({[(9H-fluoren-9-yl)methoxy]carbonyl}amino)-3-{3-[(prop-2-en-1-yloxy)carbonyl]phenyl}propanoic acid, (2R)-3-{4-[(tert-butoxy)carbonyl]phenyl}-2-({[(9H-fluoren-9-yl)methoxy]carbonyl}amino)propanoic acid, (2R)-2-({[(9H-fluoren-9-yl)methoxy]carbonyl}amino)-3-{4-[(prop-2-en-1-yloxy)carbonyl]phenyl}propanoic acid, (2S)-2-({[(9H-fluoren-9-yl)methoxy]carbonyl}amino)-3-[5-methyl-1-(triphenylmethyl)-1H-imidazol-4-yl]propanoic acid, (2R)-3-{[(2,4-dimethoxyphenyl)methyl]sulfamoyl}-2-({[(9H-fluoren-9-yl)methoxy]carbonyl}amino)propanoic acid, (S)-2-((((9H-fluoren-9-yl)methoxy)carbonyl)amino)-3-(5-phenylpyridin-3-yl)propanoic acid, (S)-2-((((9H-fluoren-9-yl)methoxy)carbonyl)(methyl)amino)-3-(3,4-dichlorophenyl)propanoic acid, (S)-2-((((9H-fluoren-9-yl)methoxy)carbonyl)amino)-3-(4-(2-amino-2-oxoethyl)phenyl)propanoic acid, (S)-2-((((9H-fluoren-9-yl)methoxy)carbonyl)amino)-3-(2-carbamoylpyridin-3-yl)propanoic acid, (S)-2-((((9H-fluoren-9-yl)methoxy)carbonyl)amino)-3-(5,6-dimethylpyridin-3-yl)propanoic acid, (S)-2-((((9H-fluoren-9-yl)methoxy)carbonyl)amino)-3-(2,6-dimethylpyridin-4-yl)propanoic acid, (R)-2-((((9H-fluoren-9-yl)methoxy)carbonyl)amino)-3-(2-((tert-butoxycarbonyl)amino)pyridin-4-yl)propanoic acid, (S)-2-((((9H-fluoren-9-yl)methoxy)carbonyl)amino)-4-(5-(trifluoromethyl)pyridin-3-yl)butanoic acid, (S)-2-((((9H-fluoren-9-yl)methoxy)carbonyl)amino)-4-(2-(trifluoromethyl)pyridin-4-yl)butanoic acid, (R)-2-((((9H-fluoren-9-yl)methoxy)carbonyl)(methyl)amino)-3-cyclobutylpropanoic acid, (S)-2-((((9H-fluoren-9-yl)methoxy)carbonyl)(methyl)amino)-3-(2,3-dichlorophenyl)propanoic acid, (S)-2-((((9H-fluoren-9-yl)methoxy)carbonyl)amino)-3-(1-trityl-1,4,5,6-tetrahydrocyclopenta[c]pyrazol-3-yl)propanoic acid, (S)-2-((((9H-fluoren-9-yl)methoxy)carbonyl)amino)-3-(4-(tetrahydro-2H-pyran-4-yl)phenyl)propanoic acid, (S)-2-((((9H-fluoren-9-yl)methoxy)carbonyl)amino)-4-(3-(pentafluoro-16-sulfaneyl)phenyl)butanoic acid, (2S,4R)-1-(((9H-fluoren-9-yl)methoxy)carbonyl)-4-(2-chlorophenoxy)pyrrolidine-2-carboxylic acid, (2S,4R)-1-(((9H-fluoren-9-yl)methoxy)carbonyl)-4-(2-fluorophenoxy)pyrrolidine-2-carboxylic acid, (2S,4R)-1-(((9H-fluoren-9-yl)methoxy)carbonyl)-4-(3-chlorophenoxy)pyrrolidine-2-carboxylic acid, (2S,4R)-1-(((9H-fluoren-9-yl)methoxy)carbonyl)-4-(3-fluorophenoxy)pyrrolidine-2-carboxylic acid, (2S,4R)-1-(((9H-fluoren-9-yl)methoxy)carbonyl)-4-(4-chlorophenoxy)pyrrolidine-2-carboxylic acid, (2S,4R)-1-(((9H-fluoren-9-yl)methoxy)carbonyl)-4-(4-fluorophenoxy)pyrrolidine-2-carboxylic acid, (2S)-2-({[(9H-fluoren-9-yl)methoxy]carbonyl}amino)-3-{2-fluoro-[1,1′-biphenyl]-4-yl}propanoic acid, (S)-2-((((9H-fluoren-9-yl)methoxy)carbonyl)amino)-3-(6-(2-((tert-butoxycarbonyl)amino)ethoxy)pyridin-3-yl)propanoic acid, (S)-2-((((9H-fluoren-9-yl)methoxy)carbonyl)amino)-4-(dimethylamino)butanoic acid hydrochloride, (S)-2-((((9H-fluoren-9-yl)methoxy)carbonyl)amino)-3-(4-(quinolin-4-yl)phenyl)propanoic acid, (S,Z)-2-((((9H-fluoren-9-yl)methoxy)carbonyl)amino)-3-(4-(2-methyl-3-((2,2,4,6,7-pentamethyl-2,3-dihydrobenzofuran-5-yl)sulfonyl)guanidino)phenyl)propanoic acid, (S)-2-((((9H-fluoren-9-yl)methoxy)carbonyl)amino)-3-(4-((1-((2,2,4,6,7-pentamethyl-2,3-dihydrobenzofuran-5-yl)sulfonyl)-4,5-dihydro-1H-imidazol-2-yl)amino)phenyl)propanoic acid, (S)-2-((((9H-fluoren-9-yl)methoxy)carbonyl)amino)-3-(2-(dimethylcarbamoyl)-1-((2-(trimethylsilyl)ethoxy)methyl)-1H-imidazol-4-yl)propanoic acid, (S)-2-((((9H-fluoren-9-yl)methoxy)carbonyl)amino)-3-(2-(methylcarbamoyl)-1-((2-(trimethylsilyl)ethoxy)methyl)-1H-imidazol-4-yl)propanoic acid, (2S)-3-[({1-[(tert-butoxy)carbonyl]azetidin-3-yl}methyl)carbamoyl]-2-({[(9H-fluoren-9-yl)methoxy]carbonyl}amino)propanoic acid, (S)-2-((((9H-fluoren-9-yl)methoxy)carbonyl)amino)-3-(isoquinolin-8-yl)propanoic acid, (2S,4R)-1-(((9H-fluoren-9-yl)methoxy)carbonyl)-4-(pyridin-2-yloxy)pyrrolidine-2-carboxylic acid, (2S,4R)-1-(((9H-fluoren-9-yl)methoxy)carbonyl)-4-(pyridin-3-yloxy)pyrrolidine-2-carboxylic acid, (S)-2-((((9H-fluoren-9-yl)methoxy)carbonyl)(methyl)amino)-3-(thiazol-2-yl)propanoic acid, (S)-2-((((9H-fluoren-9-yl)methoxy)carbonyl)(methyl)amino)-3-(5-chloropyridin-3-yl)propanoic acid, (S)-2-((((9H-fluoren-9-yl)methoxy)carbonyl)(methyl)amino)-3-(5-(trifluoromethyl)pyridin-3-yl)propanoic acid, (2S)-3-(2,6-dichloropyridin-4-yl)-2-({[(9H-fluoren-9-yl)methoxy]carbonyl}(methyl)amino)propanoic acid, (2S)-2-({[(9H-fluoren-9-yl)methoxy]carbonyl}(methyl)amino)-3-(oxan-4-yl)propanoic acid, (2R)-2-({[(9H-fluoren-9-yl)methoxy]carbonyl}(methyl)amino)-3-{1-[(prop-2-en-1-yloxy)carbonyl]piperidin-4-yl}propanoic acid, (2R)-2-({[(9H-fluoren-9-yl)methoxy]carbonyl}(methyl)amino)-3-(oxan-4-yl)propanoic acid, (2R)-6-amino-2-({[(9H-fluoren-9-yl)methoxy]carbonyl}(methyl)amino)hexanoic acid hydrochloride, (2R)-2-({[(9H-fluoren-9-yl)methoxy]carbonyl}(methyl)amino)-6-{[(prop-2-en-1-yloxy)carbonyl]amino}hexanoic acid, (2S)-3-(2,3-dimethylphenyl)-2-({[(9H-fluoren-9-yl)methoxy]carbonyl}(methyl)amino)propanoic acid, (2S)-3-[3,5-bis(trifluoromethyl)phenyl]-2-({[(9H-fluoren-9-yl)methoxy]carbonyl}(methyl)amino)propanoic acid, (2S)-3-[3-chloro-5-(trifluoromethyl)phenyl]-2-({[(9H-fluoren-9-yl)methoxy]carbonyl}(methyl)amino)propanoic acid, (2S)-3-(3-ethylphenyl)-2-({[(9H-fluoren-9-yl)methoxy]carbonyl}(methyl)amino)propanoic acid, (2S)-2-({[(9H-fluoren-9-yl)methoxy]carbonyl}(methyl)amino)-6,6,6-trifluorohexanoic acid, (2S)-5-{[(tert-butoxy)carbonyl](methyl)amino}-2-({[(9H-fluoren-9-yl)methoxy]carbonyl}(methyl)amino)pentanoic acid, (2S)-2-({[(9H-fluoren-9-yl)methoxy]carbonyl}(methyl)amino)-5-{methyl[(prop-2-en-1-yloxy)carbonyl]amino}pentanoic acid, (2S)-4-(cyclohexyloxy)-2-({[(9H-fluoren-9-yl)methoxy]carbonyl}amino)butanoic acid, (2S)-2-({[(9H-fluoren-9-yl)methoxy]carbonyl}amino)-4-(oxan-4-yloxy)butanoic acid, (2S)-3-(2,2-dimethylpropoxy)-2-({[(9H-fluoren-9-yl)methoxy]carbonyl}amino)propanoic acid, (2S)-2-({[(9H-fluoren-9-yl)methoxy]carbonyl}(methyl)amino)-3-[2-oxo-2-(prop-2-en-1-yloxy)ethoxy]propanoic acid, (2R)-3-(3,4-dichlorophenyl)-2-({[(9H-fluoren-9-yl)methoxy]carbonyl}(methyl)amino)propanoic acid, (2R)-2-({[(9H-fluoren-9-yl)methoxy]carbonyl}amino)-3-(6-oxo-1,6-dihydropyridin-3-yl)propanoic acid, (2R)-4-(4-{[(tert-butoxy)carbonyl]amino}phenyl)-2-({[(9H-fluoren-9-yl)methoxy]carbonyl}amino)butanoic acid, (2R)-2-({[(9H-fluoren-9-yl)methoxy]carbonyl}(methyl)amino)-3-(6-methoxypyridin-3-yl)propanoic acid, (2S)-2-({[(9H-fluoren-9-yl)methoxy]carbonyl}(methyl)amino)-3-(2-methoxypyridin-4-yl)propanoic acid, (2R)-2-({[(9H-fluoren-9-yl)methoxy]carbonyl}(methyl)amino)-3-(6-oxo-1,6-dihydropyridin-3-yl)propanoic acid, (2S)-3-{3-[(tert-butoxy)carbonyl]-4-hydroxyphenyl}-2-({[(9H-fluoren-9-yl)methoxy]carbonyl}(methyl)amino)propanoic acid, (2S)-2-({[(9H-fluoren-9-yl)methoxy]carbonyl}(methyl)amino)-4-(4-fluorophenyl)butanoic acid, (2S)-2-({[(9H-fluoren-9-yl)methoxy]carbonyl}(methyl)amino)-3-(1,3-oxazol-4-yl)propanoic acid, (2S)-2-({[(9H-fluoren-9-yl)methoxy]carbonyl}(methyl)amino)-3-(1-methyl-1H-pyrazol-4-yl)propanoic acid, (2R)-2-({[(9H-fluoren-9-yl)methoxy]carbonyl}amino)-3-(5-phenylpyridin-3-yl)propanoic acid, (2R)-2-({[(9H-fluoren-9-yl)methoxy]carbonyl}amino)-3-(2-phenylpyridin-4-yl)propanoic acid, (2S)-2-({[(9H-fluoren-9-yl)methoxy]carbonyl}(methyl)amino)-3-(quinolin-6-yl)propanoic acid, (2R)-2-({[(9H-fluoren-9-yl)methoxy]carbonyl}amino)-3-(quinolin-5-yl)propanoic acid, (2S)-2-({[(9H-fluoren-9-yl)methoxy]carbonyl}amino)-3-(isoquinolin-7-yl)propanoic acid, (2S)-2-({[(9H-fluoren-9-yl)methoxy]carbonyl}(methyl)amino)-3-(isoquinolin-8-yl)propanoic acid, (2R,4R)-4-{[(tert-butoxy)carbonyl]amino}-1-{[(9H-fluoren-9-yl)methoxy]carbonyl}pyrrolidine-2-carboxylic acid, (2S)-4-ethoxy-2-({[(9H-fluoren-9-yl)methoxy]carbonyl}(methyl)amino)butanoic acid, (2S)-3-butoxy-2-({[(9H-fluoren-9-yl)methoxy]carbonyl}(methyl)amino)propanoic acid, (2S)-3-{[(2,4-dimethoxyphenyl)methyl]sulfamoyl}-2-({[(9H-fluoren-9-yl)methoxy]carbonyl}amino)propanoic acid, (2S)-2-({[(9H-fluoren-9-yl)methoxy]carbonyl}(methyl)amino)-4-propoxybutanoic acid, and (2S)-2-({[(9H-fluoren-9-yl)methoxy]carbonyl}amino)-3-[4-({[(prop-2-en-1-yloxy)carbonyl]amino}methyl)phenyl]propanoic acid.

8. An enantiomer, diastereomer or racemate of the amino acid derivative according to claim 2.

9. An enantiomer, diastereomer or racemate of the amino acid derivative according to claim 3.

10. An enantiomer, diastereomer or racemate of the amino acid derivative according to claim 4.

11. An enantiomer, diastereomer or racemate of the amino acid derivative according to claim 6.

12. An enantiomer, diastereomer or racemate of the amino acid derivative according to claim 7.
